(19) United States
(12) Patent Application Publication
(10) Pub. No.: US 2025/0228133 A1
(43) Pub. Date: Jul. 10, 2025

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO, LTD, Yongin-si (KR)

(72) Inventors: Suheon IM, Suwon-si (KR); Byoungkwan LEE, Suwon-si (KR); Hanill LEE, Suwon-si (KR); Hyung Sun KIM, Suwon-si (KR); Dalho HUH, Suwon-si (KR); Byungku KIM, Suwon-si (KR); Wook KIM, Suwon-si (KR); Yoonman LEE, Suwon-si (KR); Seungjae LEE, Suwon-si (KR); Jiah YOON, Suwon-si (KR); Seonyeong GWAK, Suwon-si (KR); Mijin LEE, Suwon-si (KR); Seunghoon HWANG, Suwon-si (KR)

(21) Appl. No.: 19/006,390

(22) Filed: Dec. 31, 2024

(30) Foreign Application Priority Data
Jan. 5, 2024 (KR) 10-2024-0002491

(51) Int. Cl. H10K 85/60; C07D 487/04; C09K 11/02; H10K 50/11

(52) U.S. Cl. CPC H10K 85/6572; C07D 487/04; C09K 11/02; H10K 85/622; H10K 85/654; H10K 85/6574; H10K 50/11

(57) ABSTRACT

A compound for an organic optoelectronic device, a composition for an organic optoelectronic device including the compound for an organic optoelectronic device, an organic optoelectronic device including the compound or the composition, and a display device including the organic optoelectronic device, the compound being represented by Chemical Formula 1:

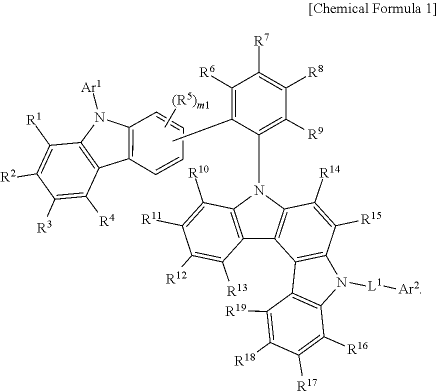

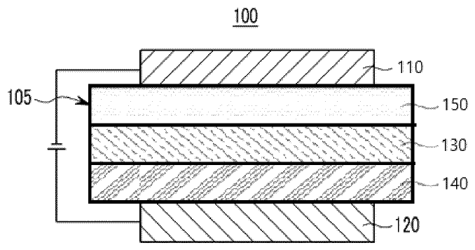

### CROSS-REFERENCE TO RELATED APPLICATION

This application claims priority to and the benefit of Korean Patent Application No. 10-2024-0002491 filed in the Korean Intellectual Property Office on Jan. 5, 2024, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode converts electrical energy into light by applying current to an organic light emitting material and performance of an organic light emitting diode may be affected by organic materials disposed between electrodes.

### SUMMARY

The embodiments may be realized by providing a compound for an organic optoelectronic device, the compound being represented by Chemical Formula 1:

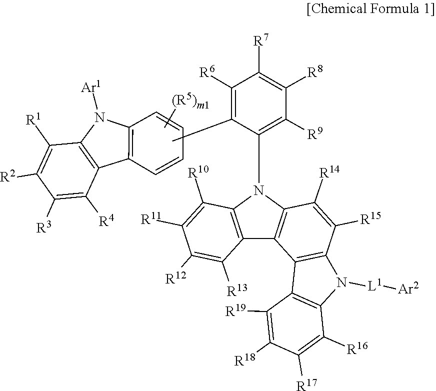

wherein, in Chemical Formula 1, L¹ is a single bond, a substituted or unsubstituted C6 to C30 arylene group, or a substituted or unsubstituted C2 to C30 heteroarylene group, Ar¹ and Ar² are each independently a substituted or unsubstituted C6 to C30 aryl group or a substituted or unsubstituted C2 to C30 heterocyclic group, R¹ to R¹⁹ are each independently hydrogen, deuterium, a cyano group, a halogen, a substituted or unsubstituted C1 to C30 alkyl group, a substituted or unsubstituted C6 to C30 aryl group, a substituted or unsubstituted C2 to C30 heterocyclic group, a substituted or unsubstituted C1 to C10 alkylsilyl group, or a combination thereof, m1 is an integer of 1 to 3, and when m1 is 2 or 3, each R⁵ is the same or different from each other.

The embodiments may be realized by providing a composition for an organic optoelectronic device, the composition including a first compound; and a second compound, wherein the first compound is the compound for an organic optoelectronic device according to an embodiment, and the second compound is represented by Chemical Formula 2:

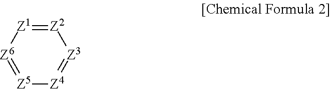

in Chemical Formula 2, Z¹ to Z⁶ are each independently N or C-L^{a}-R^{a}, provided that at least two of Z¹ to Z⁶ are N, each L^{a} is independently a single bond, a substituted or unsubstituted C6 to C20 arylene group, a substituted or unsubstituted C2 to C20 heterocyclic group, or a combination thereof, each R^{a} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and each R^{a} is separately present, or adjacent groups thereof are linked to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring.

The embodiments may be realized by providing an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing an organic optoelectronic device including an anode and a cathode facing each other, and at least one organic layer between the anode and the cathode, wherein the at least one organic layer includes the composition for an organic optoelectronic device according to an embodiment.

The embodiments may be realized by providing a display device including the organic photoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view showing an organic light emitting diode according to some example embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not necessarily an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example, the “substituted” refers to replacement of at least one hydrogen of a substituent or a compound by deuterium, a C1 to C20 alkyl group, a C6 to C30 aryl group, or a cyano group. In specific example, the “substituted” refers to replacement of at least one hydrogen of a substituent or a compound by deuterium, a C1 to C5 alkyl group, a C6 to C18 aryl group, or a cyano group. In specific example, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).” For example, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quaterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to some example embodiments is described.

A compound for an organic optoelectronic device according to some example embodiments may be represented by Chemical Formula 1.

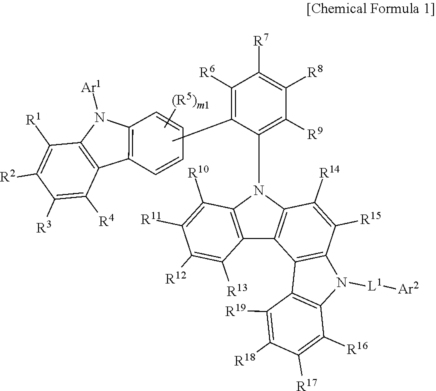

In Chemical Formula 1, L¹ may be or may include, e.g., a single bond, a substituted or unsubstituted C6 to C30 arylene group, or a substituted or unsubstituted C2 to C30 heteroarylene group.

Ar¹ and Ar² may each independently be or include, e.g., a substituted or unsubstituted C6 to C30 aryl group or a substituted or unsubstituted C2 to C30 heterocyclic group.

R¹ to R¹⁹ may each independently be or include, e.g., hydrogen, deuterium, a cyano group, a halogen, a substituted or unsubstituted C1 to C30 alkyl group, a substituted or unsubstituted C6 to C30 aryl group, a substituted or unsubstituted C2 to C30 heterocyclic group, a substituted or unsubstituted C1 to C10 alkylsilyl group, or a combination thereof.

m1 may be, e.g., an integer of 1 to 3.

The compound represented by Chemical Formula 1 may have a structure in which the indolocarbazole mother nucleus or core is additionally substituted with carbazole (e.g., through a linking phenylene group) in an N-direction of indolocarbazole.

By additionally being substituted with carbazole in the N-direction of indolocarbazole, it can have a shallow HOMO, and thus the driving voltage can be improved due to fast hole transfer characteristics. These characteristics may be due to the hole characteristics of additionally substituted carbazole, and may not be expected from structures that do not include additionally substituted carbazole.

In an implementation, the N-direction of indolocarbazole may be substituted with ortho-phenylene for indolocarbazole (e.g., as the linking group), thereby increasing steric hindrance, significantly lowering the deposition temperature, and improving thermal stability.

In an implementation, ortho-phenylene may help secure sufficient distance between molecules, thereby weakening TTA (triplet-triplet annihilation) and improving the efficiency of organic light emitting diodes using it.

In an implementation, m1 may be 2 or 3, and each R⁵ may be the same or different from each other.

In an implementation, Chemical Formula 1 may be represented by, e.g., one of Chemical Formula 1-1 to Chemical Formula 1-4.

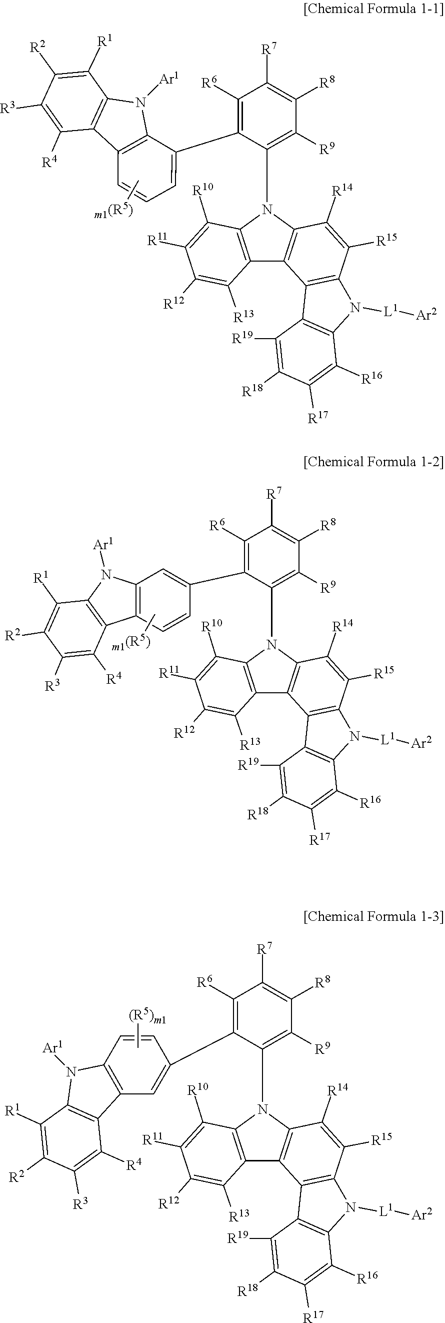

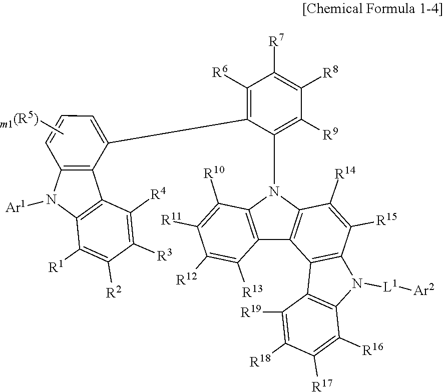

In Chemical Formula 1-1 to Chemical Formula 1-4, L¹, Ar¹ and Ar², R¹ to R¹⁹, and m1 may be defined the same as those of Chemical Formula 1.

In an implementation, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted triphenylene group, a substituted or unsubstituted dibenzofuranyl group, a substituted or unsubstituted dibenzothiophenyl group, or a substituted or unsubstituted dibenzosilolyl group.

In an implementation, moieties *-L¹-Ar² and *—Ar¹ may each independently be, e.g., a moiety of Group I.

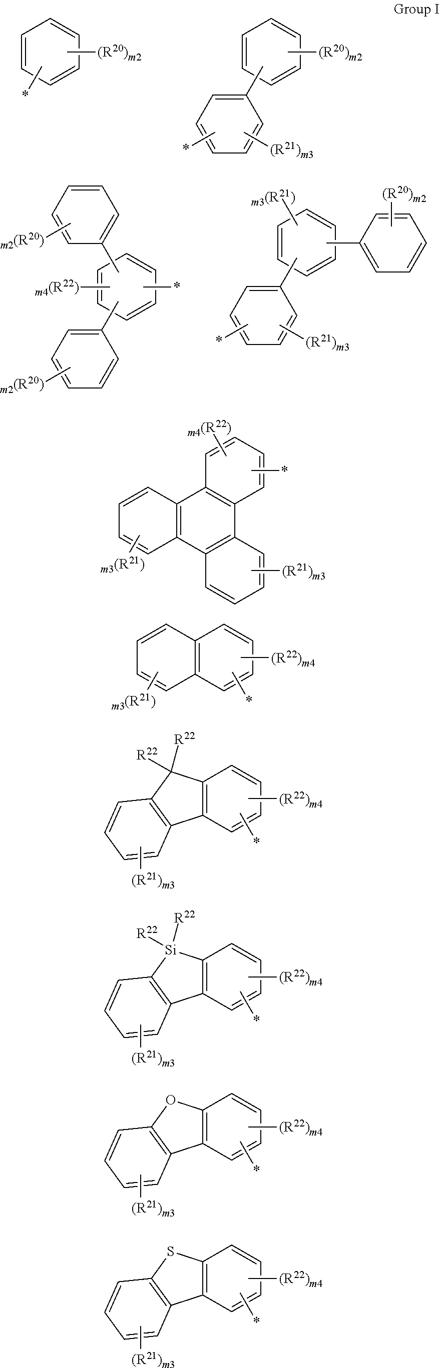

In Group I, R²⁰ to R²² may each independently be, e.g., hydrogen, deuterium, a cyano group, a substituted or unsubstituted C1 to C10 alkyl group, or a substituted or unsubstituted C6 to C12 aryl group.

m2 may be, e.g., an integer of 1 to 5.

m3 may be, e.g., an integer of 1 to 4.

m4 may be, e.g., an integer of 1 to 3.

* is a linking point.

In an implementation, m2 may be 2 to 5, and each R²⁰ may be the same or different from each other.

In an implementation, m3 may be 2 to 4, and each R²¹ may be the same or different from each other.

In an implementation, m4 may be 2 or 3, and each R²² may be the same or different from each other.

In an implementation, R¹ to R¹⁹ may each independently be, e.g., hydrogen, deuterium, a cyano group, a substituted or unsubstituted C1 to C10 alkyl group, a substituted or unsubstituted C6 to C12 aryl group, a substituted or unsubstituted C2 to C20 heterocyclic group, a substituted or unsubstituted C1 to C10 alkylsilyl group, or a combination thereof.

In an implementation, R¹ to R¹⁹ may each independently be, e.g., hydrogen, deuterium, a cyano group, a substituted or unsubstituted methyl group, a substituted or unsubstituted ethyl group, a substituted or unsubstituted propyl group, a substituted or unsubstituted butyl group, a substituted or unsubstituted phenyl group, a substituted or unsubstituted fluorenyl group, a substituted or unsubstituted dibenzofuranyl group, a substituted or unsubstituted dibenzothiophenyl group, a substituted or unsubstituted dibenzosilolyl group, or a substituted or unsubstituted trimethylsilyl group.

In an implementation, the compound for an organic optoelectronic device represented by Chemical Formula 1 may be, e.g., a compound of Group 1.

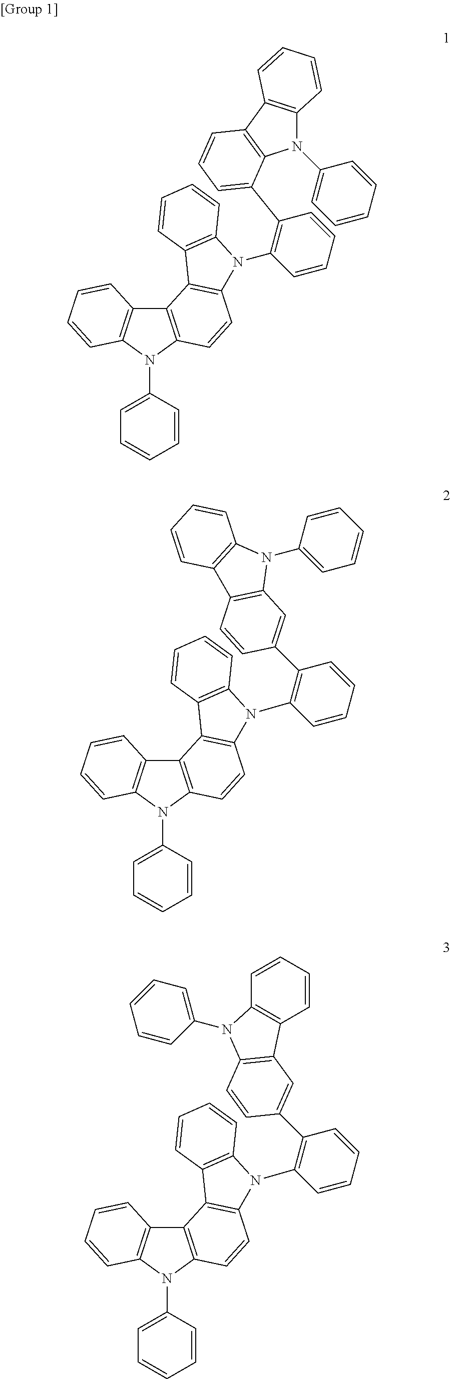 [SMILES: c1ccc(-n2c3ccccc3c3c4c5ccccc5n(-c5ccccc5-c5cccc6c7ccccc7n(-c7ccccc7)c56)c4ccc32)cc1 ; c1ccc(-n2c3ccccc3c3ccc(-c4ccccc4-n4c5ccccc5c5c6c7ccccc7n(-c7ccccc7)c6ccc54)cc32)cc1 ; c1ccc(-n2c3ccccc3c3cc(-c4ccccc4-n4c5ccccc5c5c6c7ccccc7n(-c7ccccc7)c6ccc54)ccc32)cc1]

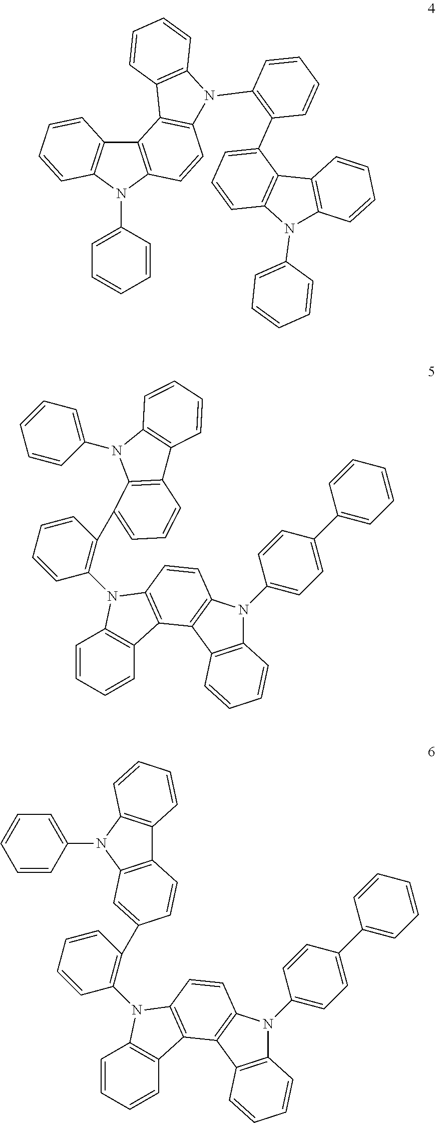 [SMILES: c1ccc(-n2c3ccccc3c3c(-c4ccccc4-n4c5ccccc5c5c6c7ccccc7n(-c7ccccc7)c6ccc54)cccc32)cc1 ; c1ccc(-c2ccc(-n3c4ccccc4c4c5c6ccccc6n(-c6ccccc6-c6cccc7c8ccccc8n(-c8ccccc8)c67)c5ccc43)cc2)cc1 ; c1ccc(-c2ccc(-n3c4ccccc4c4c5c6ccccc6n(-c6ccccc6-c6ccc7c8ccccc8n(-c8ccccc8)c7c6)c5ccc43)cc2)cc1]

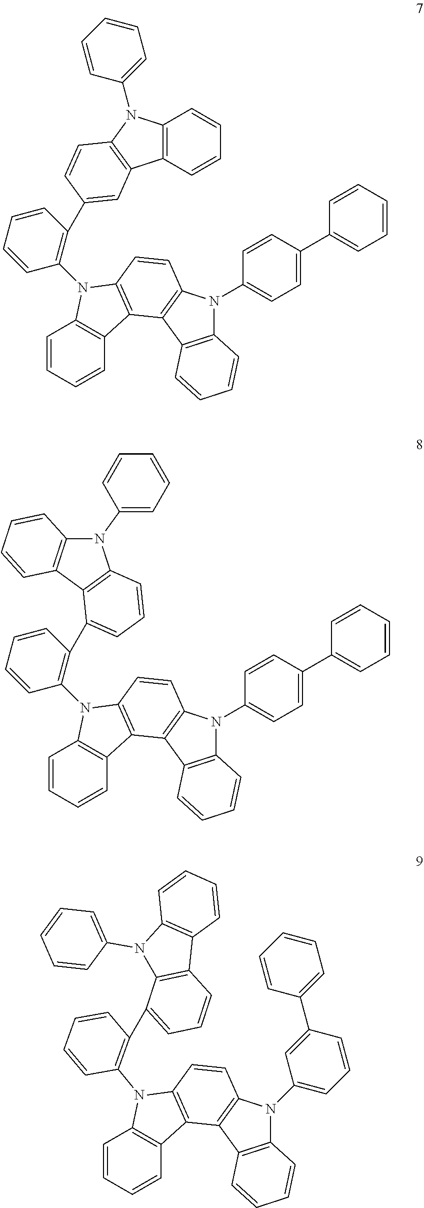 [SMILES: c1ccc(-c2ccc(-n3c4ccccc4c4c5c6ccccc6n(-c6ccccc6-c6ccc7c(c6)c6ccccc6n7-c6ccccc6)c5ccc43)cc2)cc1 ; c1ccc(-c2ccc(-n3c4ccccc4c4c5c6ccccc6n(-c6ccccc6-c6cccc7c6c6ccccc6n7-c6ccccc6)c5ccc43)cc2)cc1 ; c1ccc(-c2cccc(-n3c4ccccc4c4c5c6ccccc6n(-c6ccccc6-c6cccc7c8ccccc8n(-c8ccccc8)c67)c5ccc43)c2)cc1]

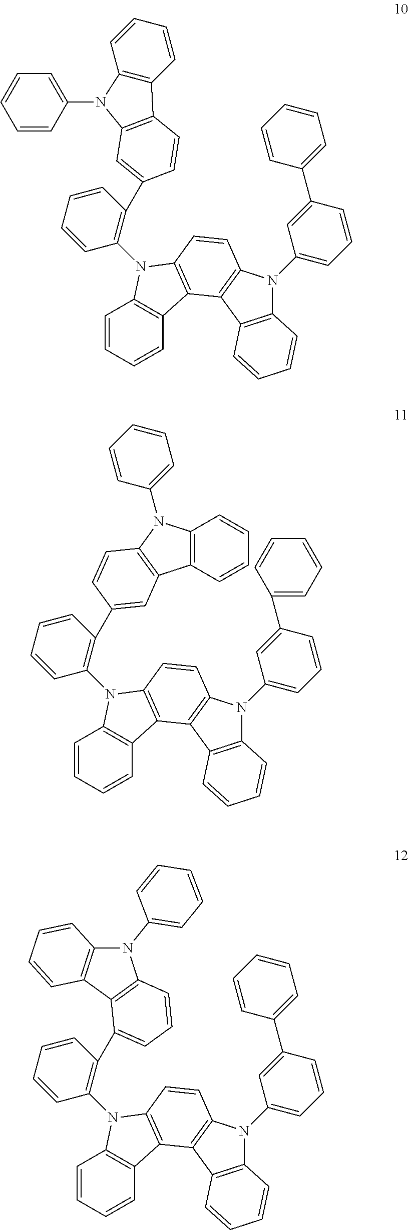 [SMILES: c1ccc(-c2cccc(-n3c4ccccc4c4c5c6ccccc6n(-c6ccccc6-c6ccc7c8ccccc8n(-c8ccccc8)c7c6)c5ccc43)c2)cc1 ; c1ccc(-c2cccc(-n3c4ccccc4c4c5c6ccccc6n(-c6ccccc6-c6ccc7c(c6)c6ccccc6n7-c6ccccc6)c5ccc43)c2)cc1 ; c1ccc(-c2cccc(-n3c4ccccc4c4c5c6ccccc6n(-c6ccccc6-c6cccc7c6c6ccccc6n7-c6ccccc6)c5ccc43)c2)cc1]

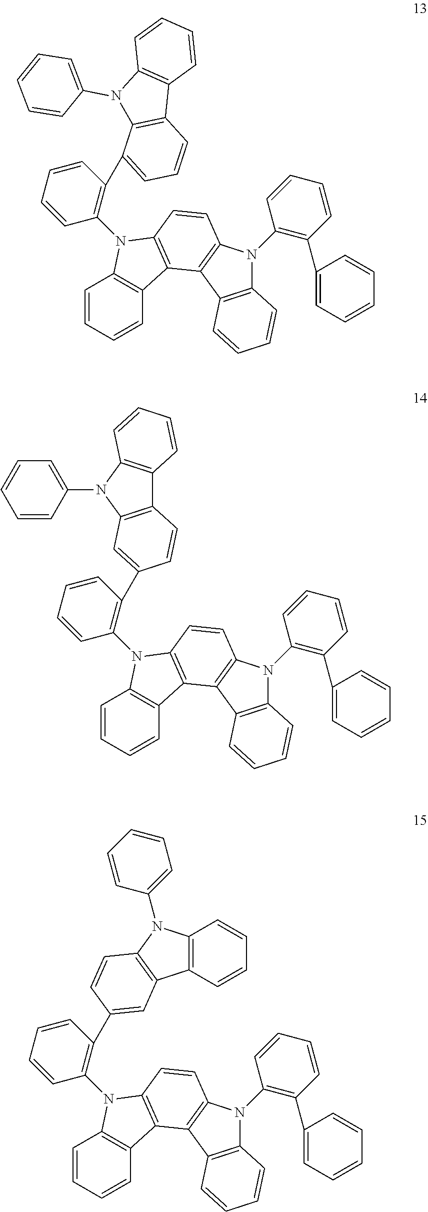 [SMILES: c1ccc(-c2ccccc2-n2c3ccccc3c3c4c5ccccc5n(-c5ccccc5-c5cccc6c7ccccc7n(-c7ccccc7)c56)c4ccc32)cc1 ; c1ccc(-c2ccccc2-n2c3ccccc3c3c4c5ccccc5n(-c5ccccc5-c5ccc6c7ccccc7n(-c7ccccc7)c6c5)c4ccc32)cc1 ; c1ccc(-c2ccccc2-n2c3ccccc3c3c4c5ccccc5n(-c5ccccc5-c5ccc6c(c5)c5ccccc5n6-c5ccccc5)c4ccc32)cc1]

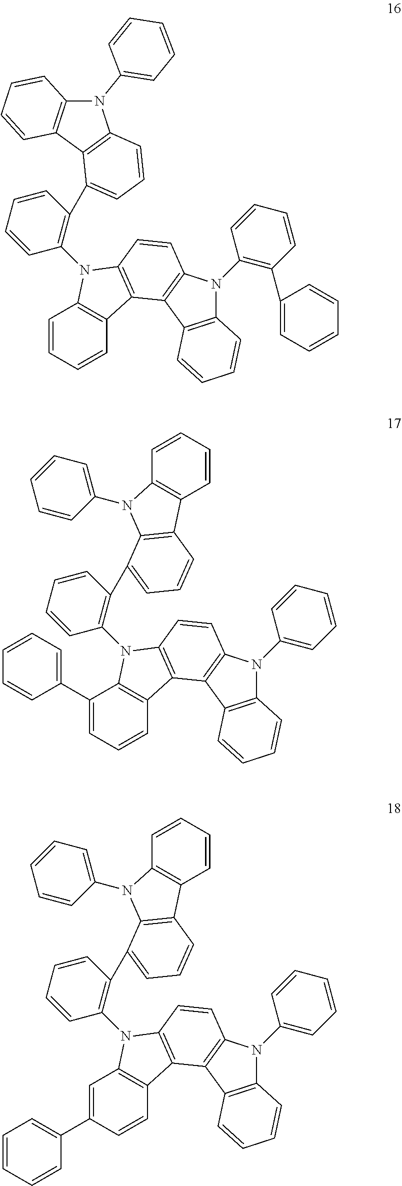 [SMILES: c1ccc(-c2ccccc2-n2c3ccccc3c3c4c5ccccc5n(-c5ccccc5-c5cccc6c5c5ccccc5n6-c5ccccc5)c4ccc32)cc1 ; c1ccc(-c2cccc3c4c5c6ccccc6n(-c6ccccc6)c5ccc4n(-c4ccccc4-c4cccc5c6ccccc6n(-c6ccccc6)c45)c23)cc1 ; c1ccc(-c2ccc3c4c5c6ccccc6n(-c6ccccc6)c5ccc4n(-c4ccccc4-c4cccc5c6ccccc6n(-c6ccccc6)c45)c3c2)cc1]

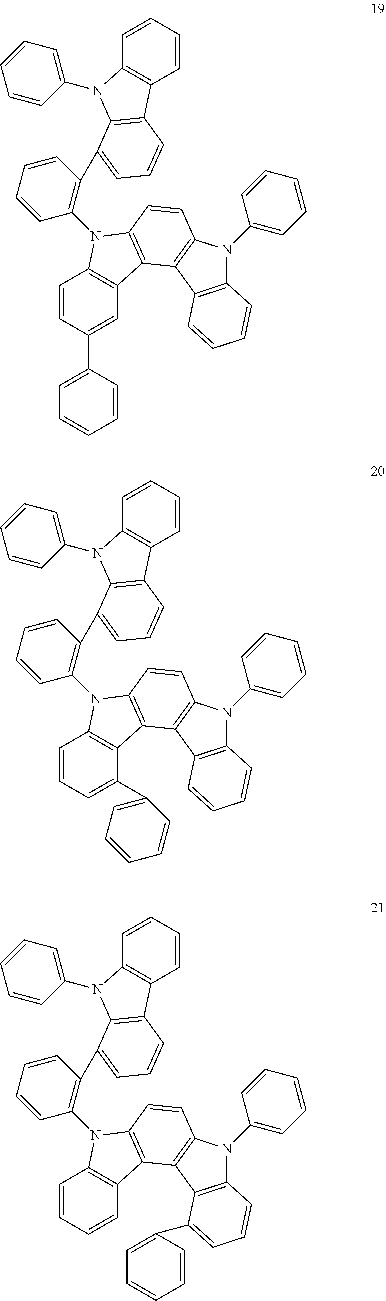 [SMILES: c1ccc(-c2ccc3c(c2)c2c4c5ccccc5n(-c5ccccc5)c4ccc2n3-c2ccccc2-c2cccc3c4ccccc4n(-c4ccccc4)c23)cc1 ; c1ccc(-c2cccc3c2c2c4c5ccccc5n(-c5ccccc5)c4ccc2n3-c2ccccc2-c2cccc3c4ccccc4n(-c4ccccc4)c23)cc1 ; c1ccc(-c2cccc3c2c2c4c5ccccc5n(-c5ccccc5-c5cccc6c7ccccc7n(-c7ccccc7)c56)c4ccc2n3-c2ccccc2)cc1]

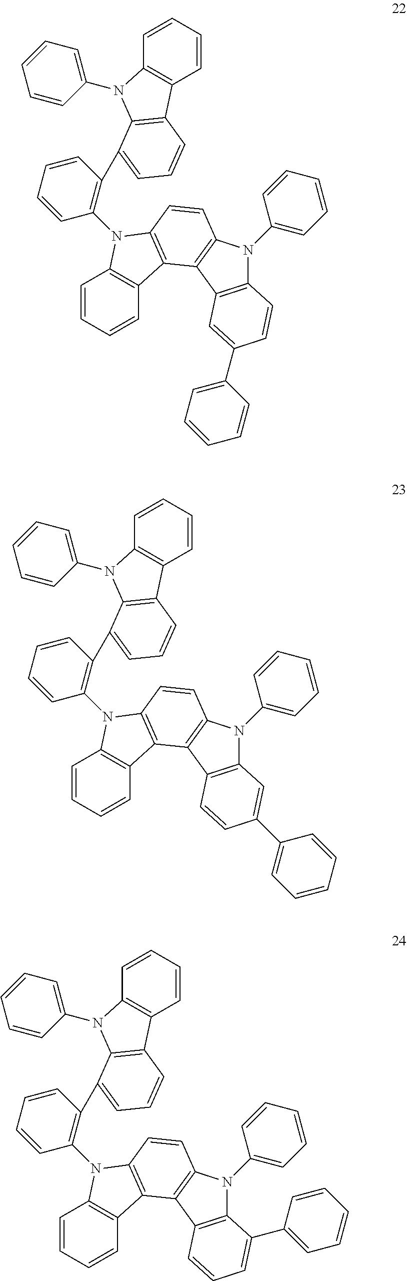 [SMILES: c1ccc(-c2ccc3c(c2)c2c4c5ccccc5n(-c5ccccc5-c5cccc6c7ccccc7n(-c7ccccc7)c56)c4ccc2n3-c2ccccc2)cc1 ; c1ccc(-c2ccc3c4c5c6ccccc6n(-c6ccccc6-c6cccc7c8ccccc8n(-c8ccccc8)c67)c5ccc4n(-c4ccccc4)c3c2)cc1 ; c1ccc(-c2cccc3c4c5c6ccccc6n(-c6ccccc6-c6cccc7c8ccccc8n(-c8ccccc8)c67)c5ccc4n(-c4ccccc4)c23)cc1]

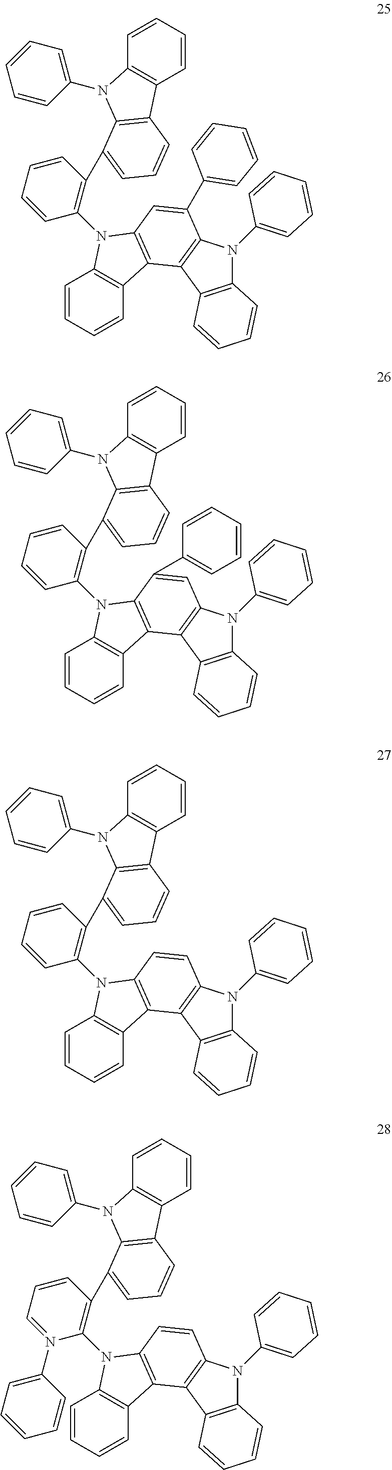

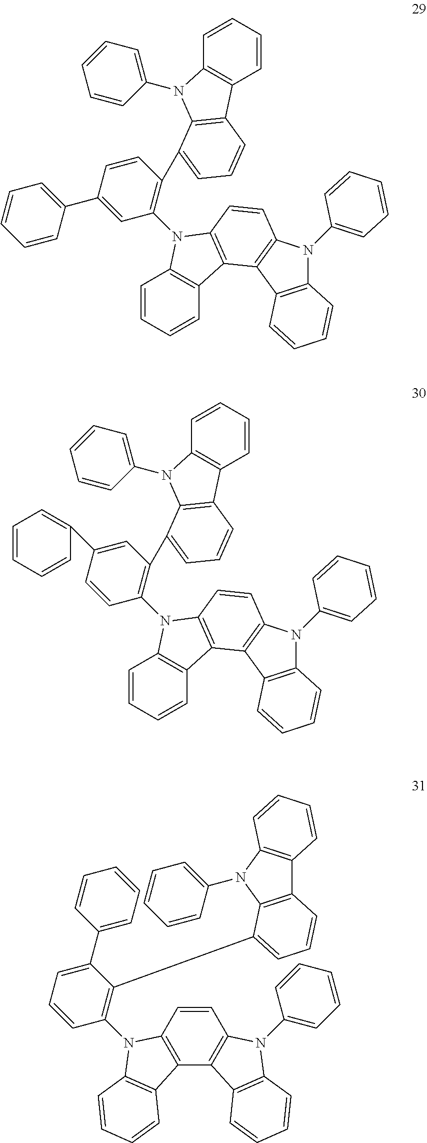 [SMILES: c1ccc(-c2ccc(-c3cccc4c5ccccc5n(-c5ccccc5)c34)c(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccccc6)c5ccc43)c(-c3cccc4c5ccccc5n(-c5ccccc5)c34)c2)cc1 ; c1ccc(-c2cccc(-n3c4ccccc4c4c5c6ccccc6n(-c6ccccc6)c5ccc43)c2-c2cccc3c4ccccc4n(-c4ccccc4)c23)cc1]

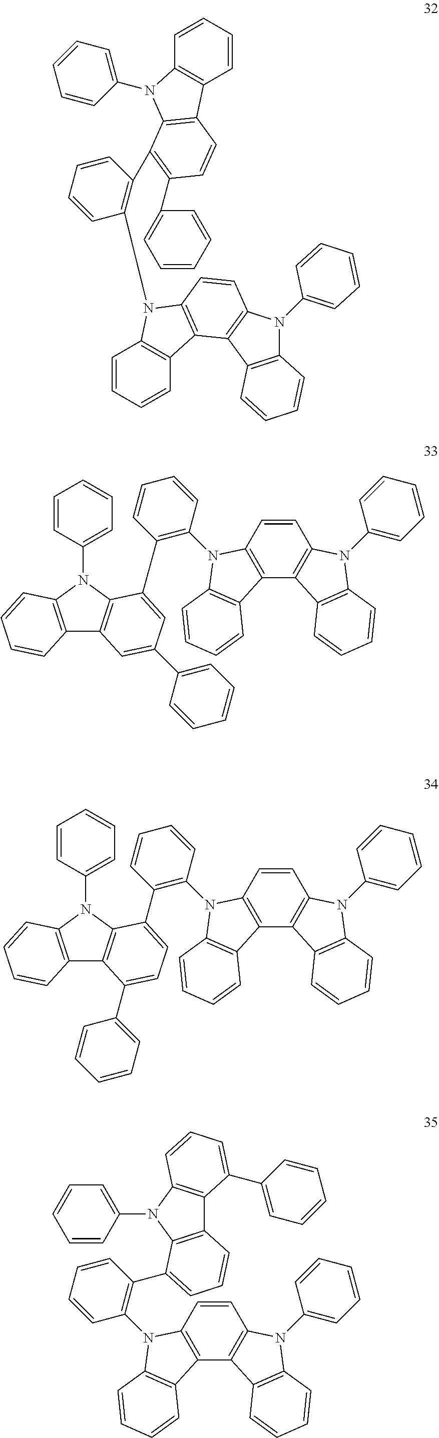 [SMILES: c1ccc(-c2ccc3c4ccccc4n(-c4ccccc4)c3c2-c2ccccc2-n2c3ccccc3c3c4c5ccccc5n(-c5ccccc5)c4ccc32)cc1 ; c1ccc(-c2cc(-c3ccccc3-n3c4ccccc4c4c5c6ccccc6n(-c6ccccc6)c5ccc43)c3c(c2)c2ccccc2n3-c2ccccc2)cc1 ; c1ccc(-c2ccc(-c3ccccc3-n3c4ccccc4c4c5c6ccccc6n(-c6ccccc6)c5ccc43)c3c2c2ccccc2n3-c2ccccc2)cc1 ; c1ccc(-c2cccc3c2c2cccc(-c4ccccc4-n4c5ccccc5c5c6c7ccccc7n(-c7ccccc7)c6ccc54)c2n3-c2ccccc2)cc1]

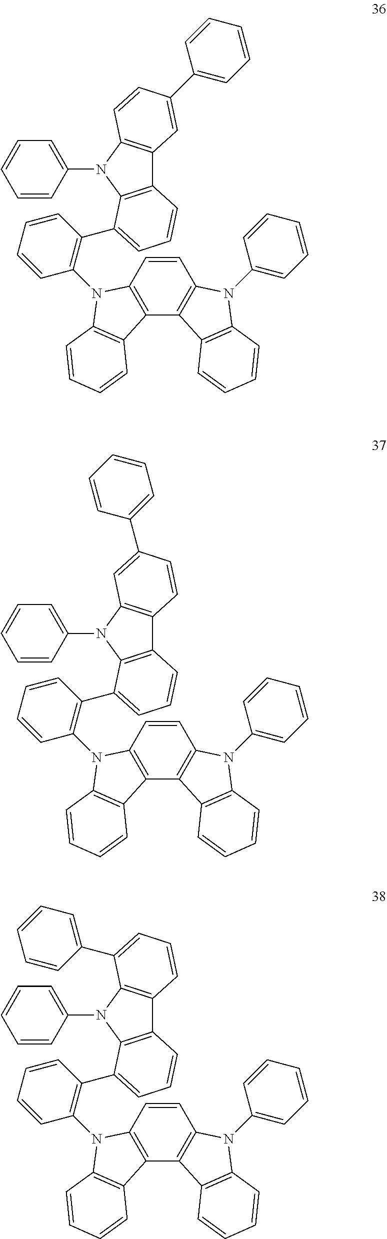 [SMILES: c1ccc(-c2ccc3c(c2)c2cccc(-c4ccccc4-n4c5ccccc5c5c6c7ccccc7n(-c7ccccc7)c6ccc54)c2n3-c2ccccc2)cc1 ; c1ccc(-c2ccc3c4cccc(-c5ccccc5-n5c6ccccc6c6c7c8ccccc8n(-c8ccccc8)c7ccc65)c4n(-c4ccccc4)c3c2)cc1 ; c1ccc(-c2cccc3c4cccc(-c5ccccc5-n5c6ccccc6c6c7c8ccccc8n(-c8ccccc8)c7ccc65)c4n(-c4ccccc4)c23)cc1]

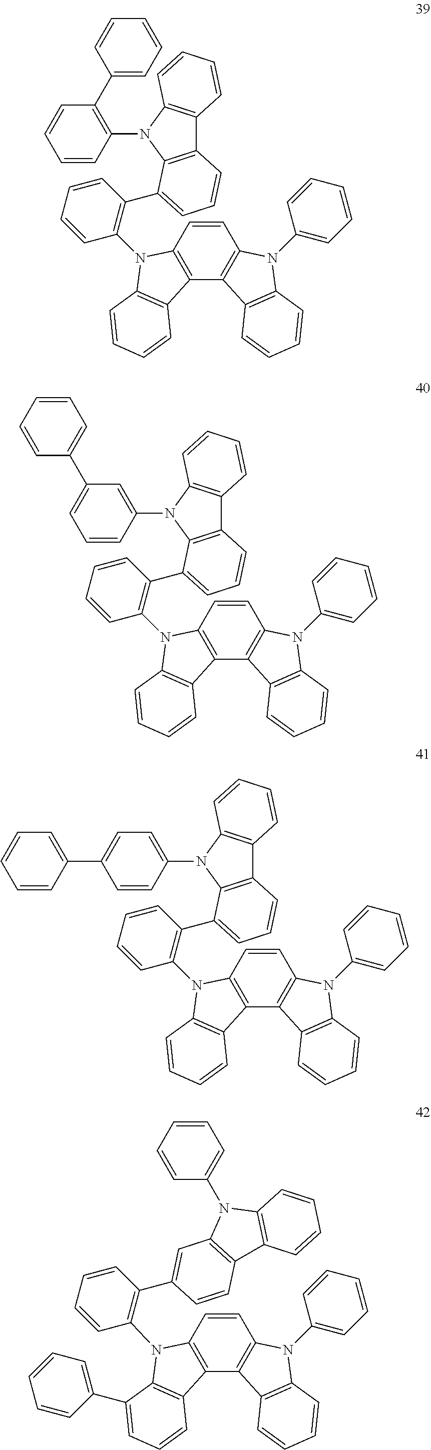

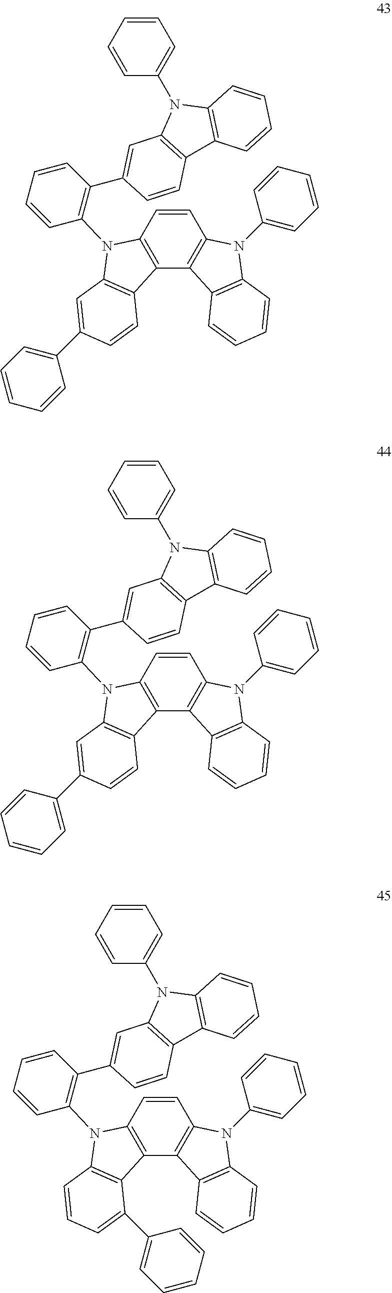 [SMILES: c1ccc(-c2ccc3c4c5c6ccccc6n(-c6ccccc6)c5ccc4n(-c4ccccc4-c4ccc5c6ccccc6n(-c6ccccc6)c5c4)c3c2)cc1 ; c1ccc(-c2ccc3c4c5c6ccccc6n(-c6ccccc6)c5ccc4n(-c4ccccc4-c4ccc5c6ccccc6n(-c6ccccc6)c5c4)c3c2)cc1 ; c1ccc(-c2cccc3c2c2c4c5ccccc5n(-c5ccccc5)c4ccc2n3-c2ccccc2-c2ccc3c4ccccc4n(-c4ccccc4)c3c2)cc1]

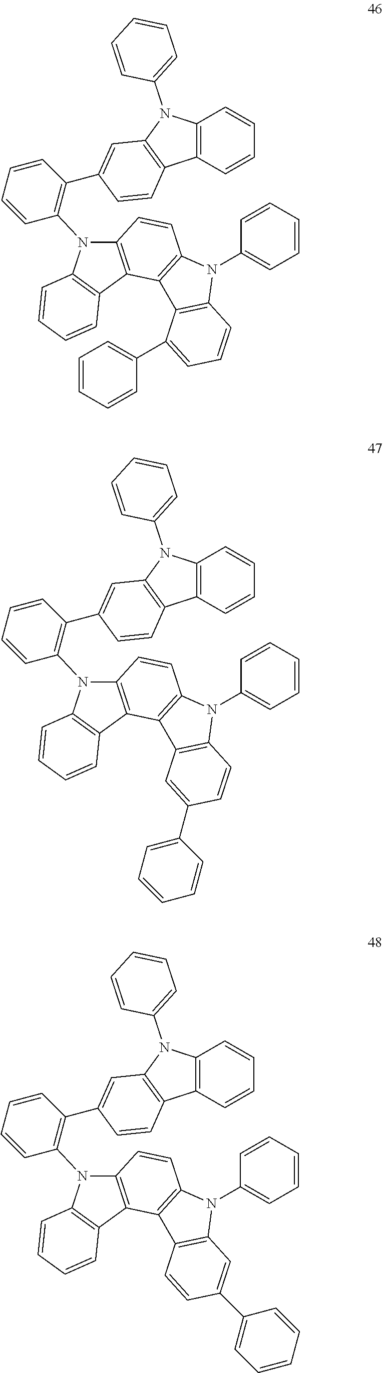 [SMILES: c1ccc(-c2cccc3c2c2c4c5ccccc5n(-c5ccccc5-c5ccc6c7ccccc7n(-c7ccccc7)c6c5)c4ccc2n3-c2ccccc2)cc1 ; c1ccc(-c2ccc3c(c2)c2c4c5ccccc5n(-c5ccccc5-c5ccc6c7ccccc7n(-c7ccccc7)c6c5)c4ccc2n3-c2ccccc2)cc1 ; c1ccc(-c2ccc3c4c5c6ccccc6n(-c6ccccc6-c6ccc7c8ccccc8n(-c8ccccc8)c7c6)c5ccc4n(-c4ccccc4)c3c2)cc1]

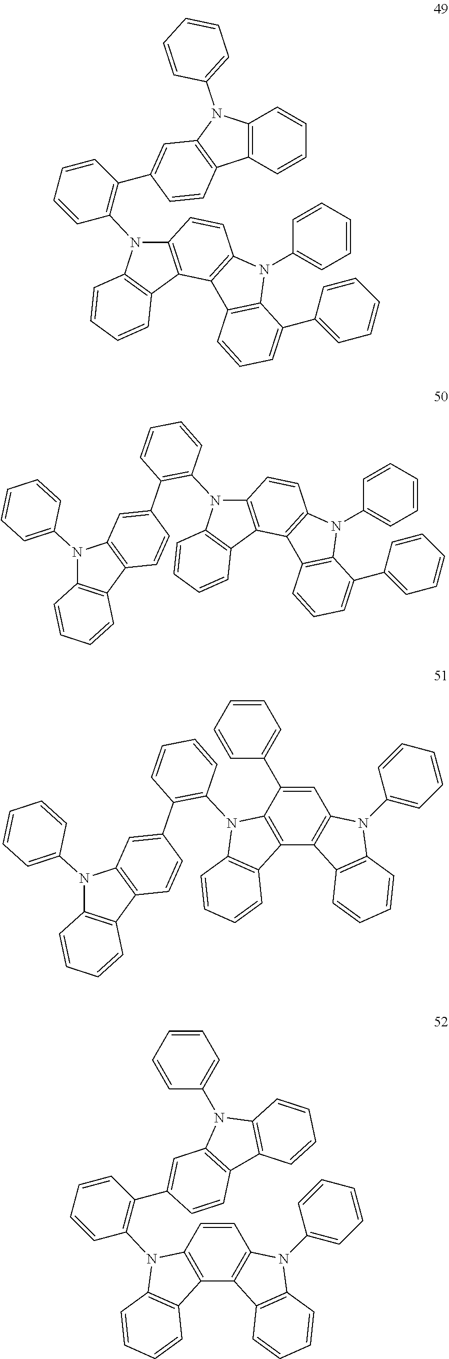 [SMILES: c1ccc(-c2cccc3c4c5c6ccccc6n(-c6ccccc6-c6ccc7c8ccccc8n(-c8ccccc8)c7c6)c5ccc4n(-c4ccccc4)c23)cc1 ; c1ccc(-c2cccc3c4c5c6ccccc6n(-c6ccccc6-c6ccc7c8ccccc8n(-c8ccccc8)c7c6)c5ccc4n(-c4ccccc4)c23)cc1 ; c1ccc(-c2cc3c(c4ccccc4n3-c3ccccc3)c3c4ccccc4n(-c4ccccc4-c4ccc5c6ccccc6n(-c6ccccc6)c5c4)c23)cc1 ; c1ccc(-n2c3ccccc3c3ccc(-c4ccccc4-n4c5ccccc5c5c6c7ccccc7n(-c7ccccc7)c6ccc54)cc32)cc1]

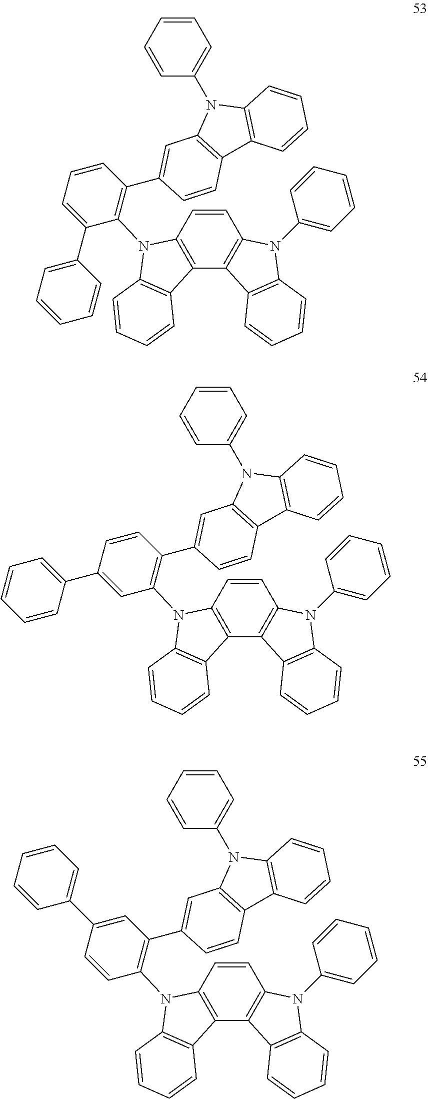 [SMILES: c1ccc(-c2cccc(-c3ccc4c5ccccc5n(-c5ccccc5)c4c3)c2-n2c3ccccc3c3c4c5ccccc5n(-c5ccccc5)c4ccc32)cc1 ; c1ccc(-c2ccc(-c3ccc4c5ccccc5n(-c5ccccc5)c4c3)c(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccccc6)c5ccc43)c(-c3ccc4c5ccccc5n(-c5ccccc5)c4c3)c2)cc1]

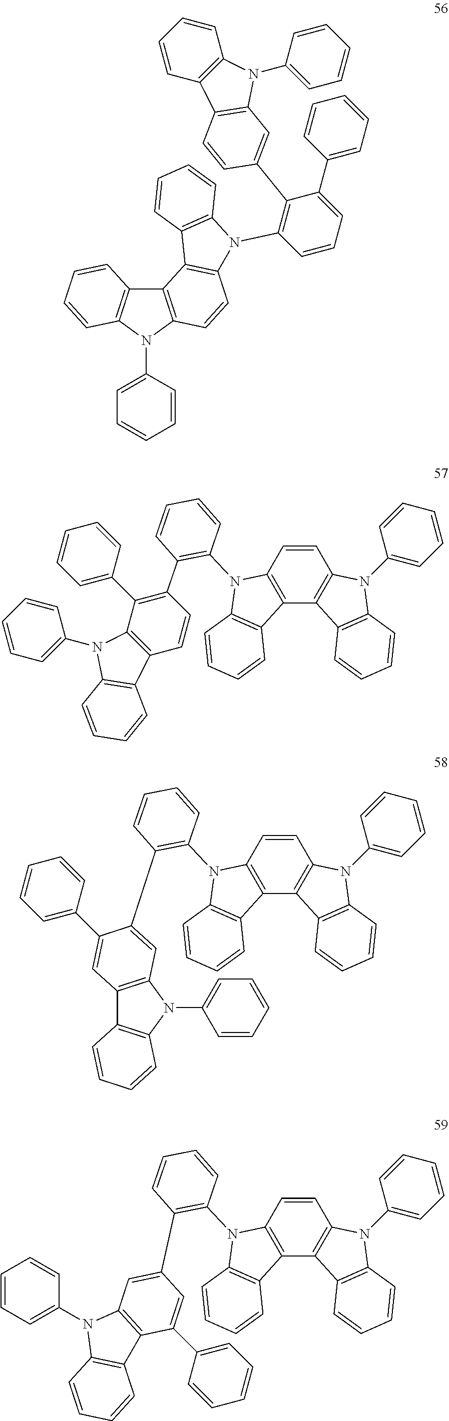 [SMILES: c1ccc(-c2cccc(-n3c4ccccc4c4c5c6ccccc6n(-c6ccccc6)c5ccc43)c2-c2ccc3c4ccccc4n(-c4ccccc4)c3c2)cc1 ; c1ccc(-c2c(-c3ccccc3-n3c4ccccc4c4c5c6ccccc6n(-c6ccccc6)c5ccc43)ccc3c4ccccc4n(-c4ccccc4)c23)cc1 ; c1ccc(-c2cc3c4ccccc4n(-c4ccccc4)c3cc2-c2ccccc2-n2c3ccccc3c3c4c5ccccc5n(-c5ccccc5)c4ccc32)cc1 ; c1ccc(-c2cc(-c3ccccc3-n3c4ccccc4c4c5c6ccccc6n(-c6ccccc6)c5ccc43)cc3c2c2ccccc2n3-c2ccccc2)cc1]

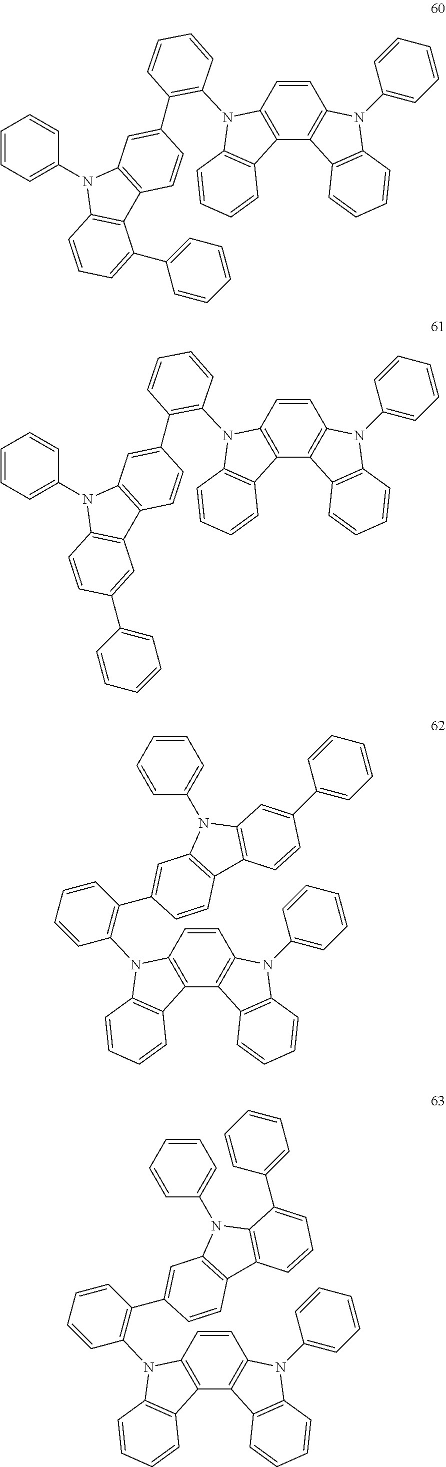 [SMILES: c1ccc(-c2cccc3c2c2ccc(-c4ccccc4-n4c5ccccc5c5c6c7ccccc7n(-c7ccccc7)c6ccc54)cc2n3-c2ccccc2)cc1 ; c1ccc(-c2ccc3c(c2)c2ccc(-c4ccccc4-n4c5ccccc5c5c6c7ccccc7n(-c7ccccc7)c6ccc54)cc2n3-c2ccccc2)cc1 ; c1ccc(-c2ccc3c4ccc(-c5ccccc5-n5c6ccccc6c6c7c8ccccc8n(-c8ccccc8)c7ccc65)cc4n(-c4ccccc4)c3c2)cc1 ; c1ccc(-c2cccc3c4ccc(-c5ccccc5-n5c6ccccc6c6c7c8ccccc8n(-c8ccccc8)c7ccc65)cc4n(-c4ccccc4)c23)cc1]

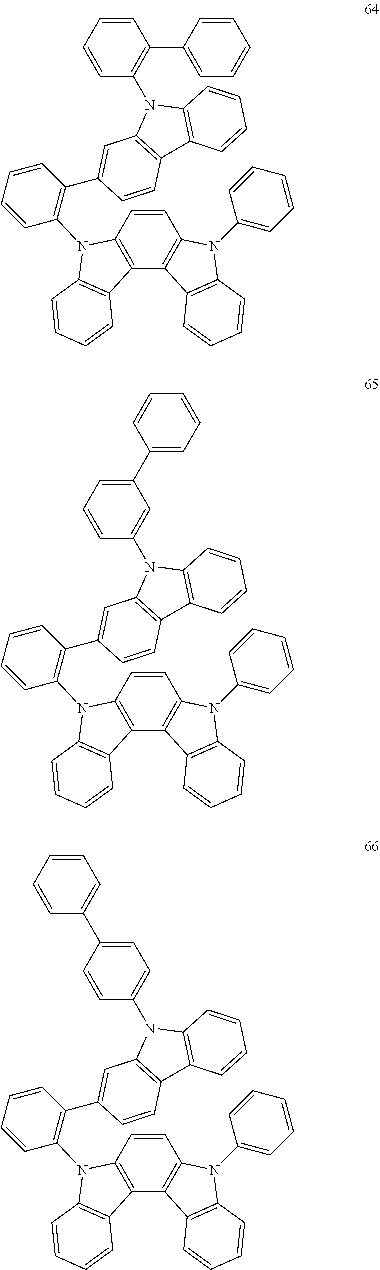 [SMILES: c1ccc(-c2ccccc2-n2c3ccccc3c3ccc(-c4ccccc4-n4c5ccccc5c5c6c7ccccc7n(-c7ccccc7)c6ccc54)cc32)cc1 ; c1ccc(-c2cccc(-n3c4ccccc4c4ccc(-c5ccccc5-n5c6ccccc6c6c7c8ccccc8n(-c8ccccc8)c7ccc65)cc43)c2)cc1 ; c1ccc(-c2ccc(-n3c4ccccc4c4ccc(-c5ccccc5-n5c6ccccc6c6c7c8ccccc8n(-c8ccccc8)c7ccc65)cc43)cc2)cc1]

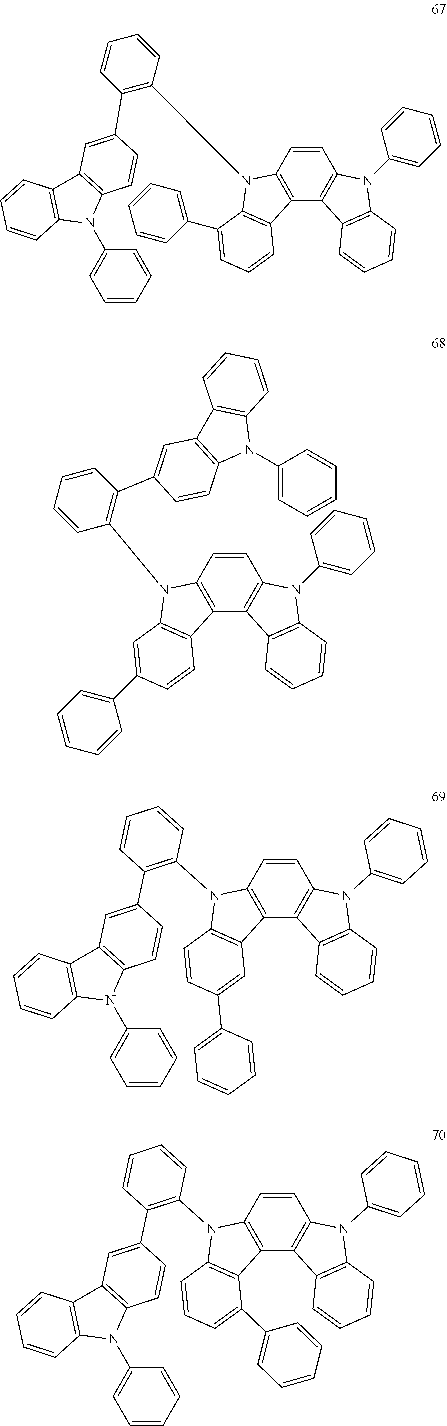 [SMILES: c1ccc(-c2cccc3c4c5c6ccccc6n(-c6ccccc6)c5ccc4n(-c4ccccc4-c4ccc5c(c4)c4ccccc4n5-c4ccccc4)c23)cc1 ; c1ccc(-c2ccc3c4c5c6ccccc6n(-c6ccccc6)c5ccc4n(-c4ccccc4-c4ccc5c(c4)c4ccccc4n5-c4ccccc4)c3c2)cc1 ; c1ccc(-c2ccc3c(c2)c2c4c5ccccc5n(-c5ccccc5)c4ccc2n3-c2ccccc2-c2ccc3c(c2)c2ccccc2n3-c2ccccc2)cc1 ; c1ccc(-c2cccc3c2c2c4c5ccccc5n(-c5ccccc5)c4ccc2n3-c2ccccc2-c2ccc3c(c2)c2ccccc2n3-c2ccccc2)cc1]

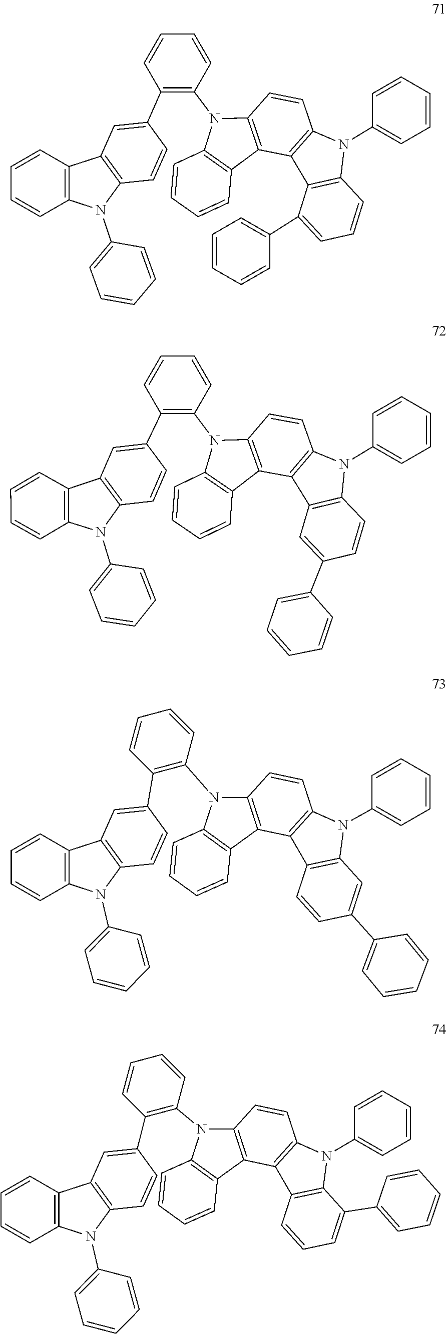 [SMILES: c1ccc(-c2cccc3c2c2c4c5ccccc5n(-c5ccccc5-c5ccc6c(c5)c5ccccc5n6-c5ccccc5)c4ccc2n3-c2ccccc2)cc1 ; c1ccc(-c2ccc3c(c2)c2c4c5ccccc5n(-c5ccccc5-c5ccc6c(c5)c5ccccc5n6-c5ccccc5)c4ccc2n3-c2ccccc2)cc1 ; c1ccc(-c2ccc3c4c5c6ccccc6n(-c6ccccc6-c6ccc7c(c6)c6ccccc6n7-c6ccccc6)c5ccc4n(-c4ccccc4)c3c2)cc1 ; c1ccc(-c2cccc3c4c5c6ccccc6n(-c6ccccc6-c6ccc7c(c6)c6ccccc6n7-c6ccccc6)c5ccc4n(-c4ccccc4)c23)cc1]

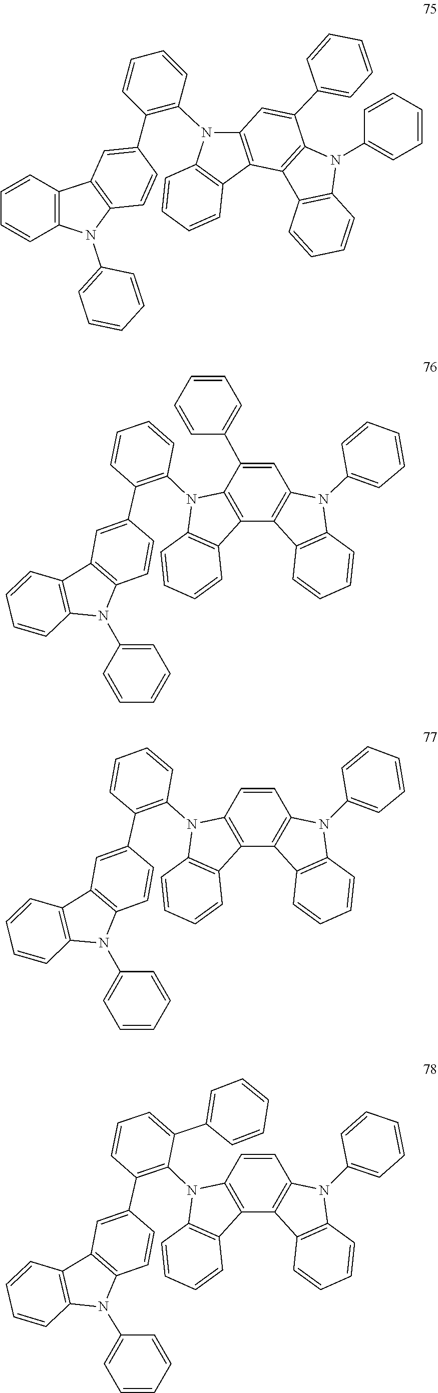

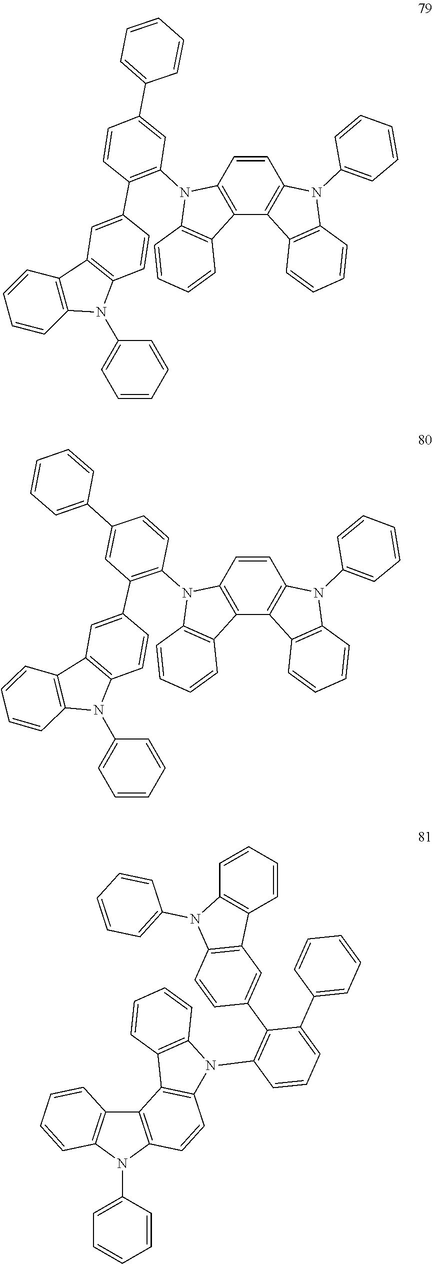 [SMILES: c1ccc(-c2ccc(-c3ccc4c(c3)c3ccccc3n4-c3ccccc3)c(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccccc6)c5ccc43)c(-c3ccc4c(c3)c3ccccc3n4-c3ccccc3)c2)cc1 ; c1ccc(-c2cccc(-n3c4ccccc4c4c5c6ccccc6n(-c6ccccc6)c5ccc43)c2-c2ccc3c(c2)c2ccccc2n3-c2ccccc2)cc1]

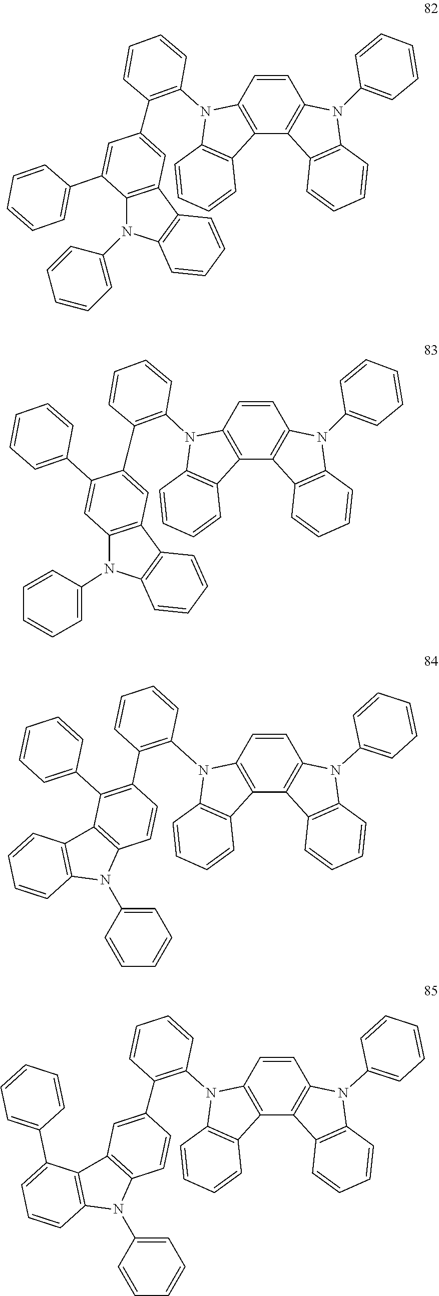 [SMILES: c1ccc(-c2cc(-c3ccccc3-n3c4ccccc4c4c5c6ccccc6n(-c6ccccc6)c5ccc43)cc3c4ccccc4n(-c4ccccc4)c23)cc1 ; c1ccc(-c2cc3c(cc2-c2ccccc2-n2c4ccccc4c4c5c6ccccc6n(-c6ccccc6)c5ccc42)c2ccccc2n3-c2ccccc2)cc1 ; c1ccc(-c2c(-c3ccccc3-n3c4ccccc4c4c5c6ccccc6n(-c6ccccc6)c5ccc43)ccc3c2c2ccccc2n3-c2ccccc2)cc1 ; c1ccc(-c2cccc3c2c2cc(-c4ccccc4-n4c5ccccc5c5c6c7ccccc7n(-c7ccccc7)c6ccc54)ccc2n3-c2ccccc2)cc1]

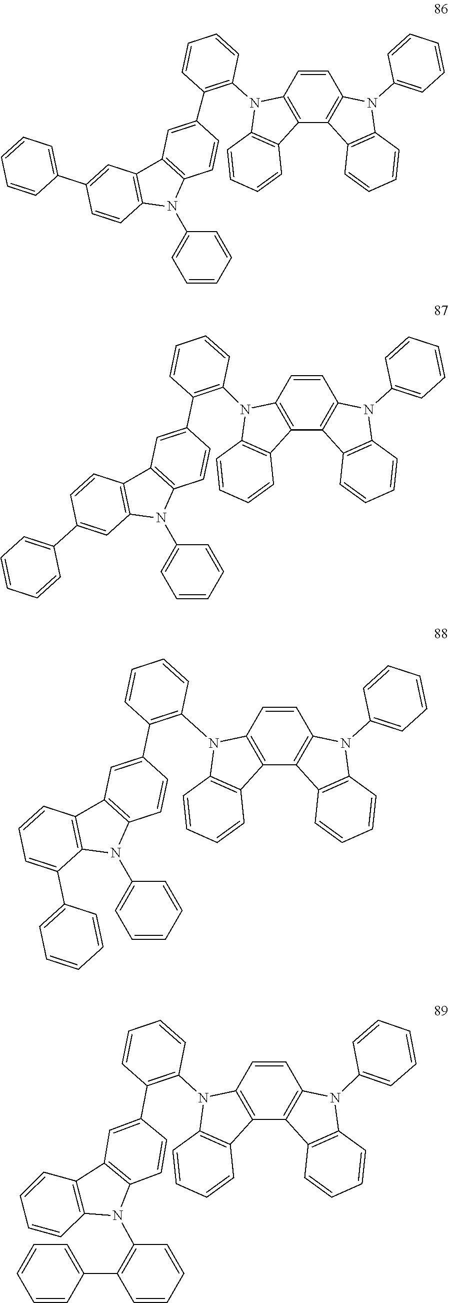 [SMILES: c1ccc(-c2ccc3c(c2)c2cc(-c4ccccc4-n4c5ccccc5c5c6c7ccccc7n(-c7ccccc7)c6ccc54)ccc2n3-c2ccccc2)cc1 ; c1ccc(-c2ccc3c4cc(-c5ccccc5-n5c6ccccc6c6c7c8ccccc8n(-c8ccccc8)c7ccc65)ccc4n(-c4ccccc4)c3c2)cc1 ; c1ccc(-c2cccc3c4cc(-c5ccccc5-n5c6ccccc6c6c7c8ccccc8n(-c8ccccc8)c7ccc65)ccc4n(-c4ccccc4)c23)cc1 ; c1ccc(-c2ccccc2-n2c3ccccc3c3cc(-c4ccccc4-n4c5ccccc5c5c6c7ccccc7n(-c7ccccc7)c6ccc54)ccc32)cc1]

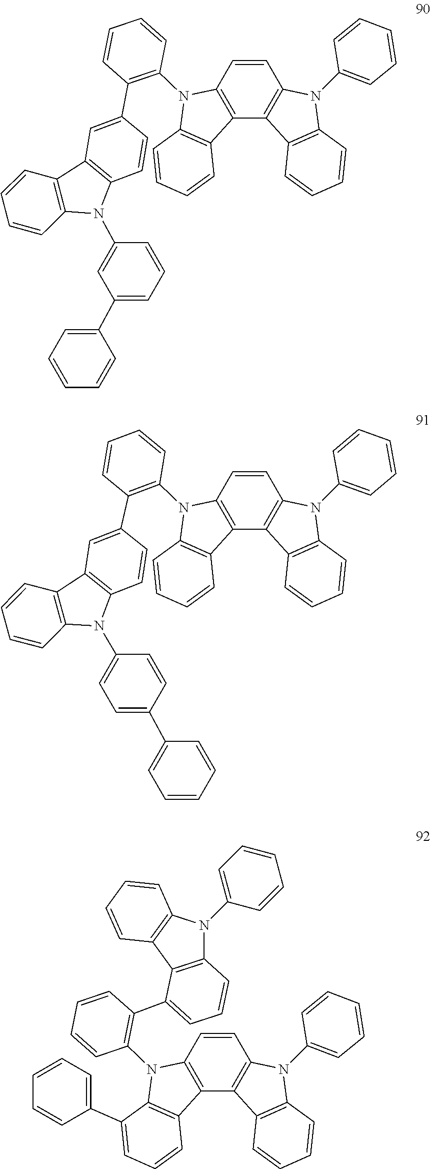 [SMILES: c1ccc(-c2cccc(-n3c4ccccc4c4cc(-c5ccccc5-n5c6ccccc6c6c7c8ccccc8n(-c8ccccc8)c7ccc65)ccc43)c2)cc1 ; c1ccc(-c2ccc(-n3c4ccccc4c4cc(-c5ccccc5-n5c6ccccc6c6c7c8ccccc8n(-c8ccccc8)c7ccc65)ccc43)cc2)cc1 ; c1ccc(-c2cccc3c4c5c6ccccc6n(-c6ccccc6)c5ccc4n(-c4ccccc4-c4cccc5c4c4ccccc4n5-c4ccccc4)c23)cc1]

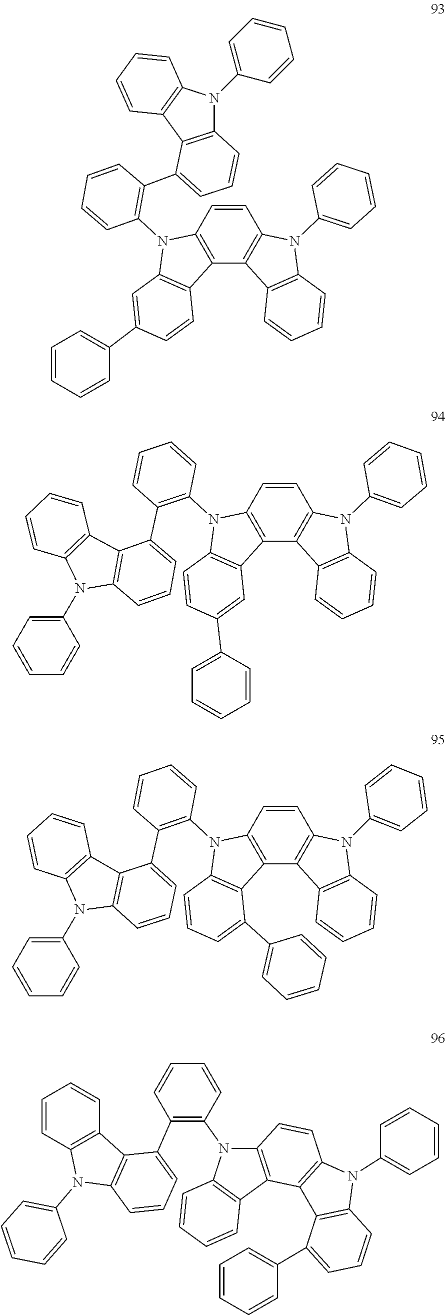 [SMILES: c1ccc(-c2ccc3c4c5c6ccccc6n(-c6ccccc6)c5ccc4n(-c4ccccc4-c4cccc5c4c4ccccc4n5-c4ccccc4)c3c2)cc1 ; c1ccc(-c2ccc3c(c2)c2c4c5ccccc5n(-c5ccccc5)c4ccc2n3-c2ccccc2-c2cccc3c2c2ccccc2n3-c2ccccc2)cc1 ; c1ccc(-c2cccc3c2c2c4c5ccccc5n(-c5ccccc5)c4ccc2n3-c2ccccc2-c2cccc3c2c2ccccc2n3-c2ccccc2)cc1 ; c1ccc(-c2cccc3c2c2c4c5ccccc5n(-c5ccccc5-c5cccc6c5c5ccccc5n6-c5ccccc5)c4ccc2n3-c2ccccc2)cc1]

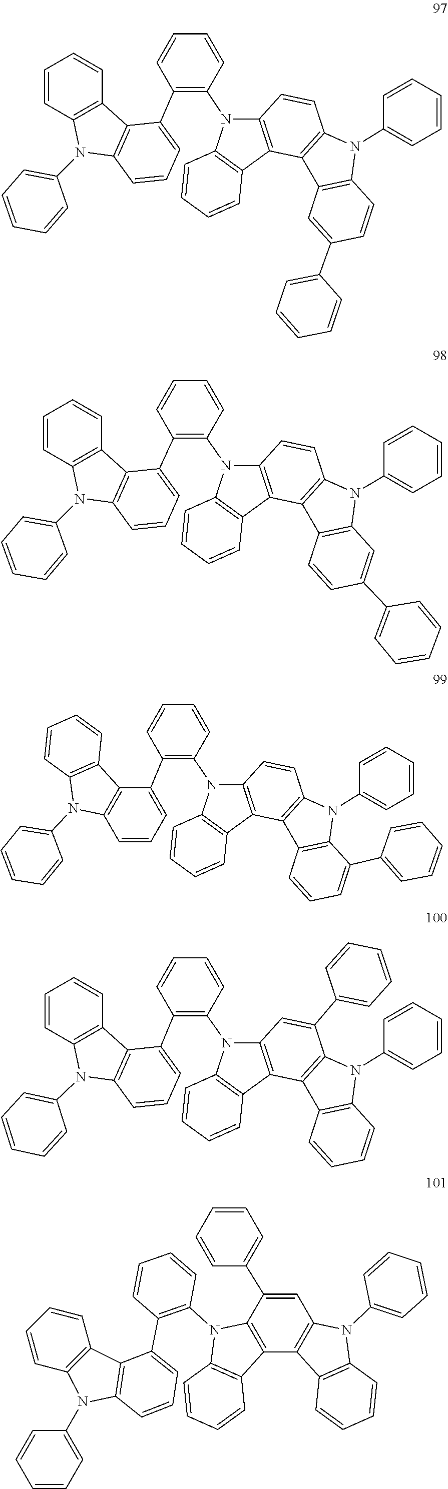 [SMILES: c1ccc(-c2ccc3c(c2)c2c4c5ccccc5n(-c5ccccc5-c5cccc6c5c5ccccc5n6-c5ccccc5)c4ccc2n3-c2ccccc2)cc1 ; c1ccc(-c2ccc3c4c5c6ccccc6n(-c6ccccc6-c6cccc7c6c6ccccc6n7-c6ccccc6)c5ccc4n(-c4ccccc4)c3c2)cc1 ; c1ccc(-c2cccc3c4c5c6ccccc6n(-c6ccccc6-c6cccc7c6c6ccccc6n7-c6ccccc6)c5ccc4n(-c4ccccc4)c23)cc1 ; c1ccc(-c2cc3c(c4ccccc4n3-c3ccccc3-c3cccc4c3c3ccccc3n4-c3ccccc3)c3c4ccccc4n(-c4ccccc4)c23)cc1 ; c1ccc(-c2cc3c(c4ccccc4n3-c3ccccc3)c3c4ccccc4n(-c4ccccc4-c4cccc5c4c4ccccc4n5-c4ccccc4)c23)cc1]

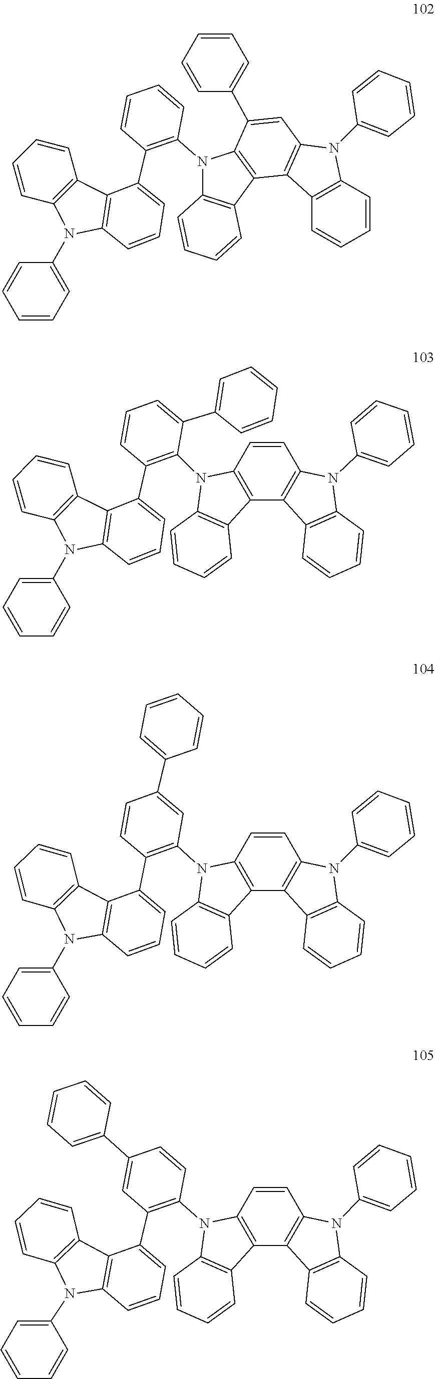 [SMILES: c1ccc(-c2cc3c(c4ccccc4n3-c3ccccc3)c3c4ccccc4n(-c4ccccc4-c4cccc5c4c4ccccc4n5-c4ccccc4)c23)cc1 ; c1ccc(-c2cccc(-c3cccc4c3c3ccccc3n4-c3ccccc3)c2-n2c3ccccc3c3c4c5ccccc5n(-c5ccccc5)c4ccc32)cc1 ; c1ccc(-c2ccc(-c3cccc4c3c3ccccc3n4-c3ccccc3)c(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccccc6)c5ccc43)c(-c3cccc4c3c3ccccc3n4-c3ccccc3)c2)cc1]

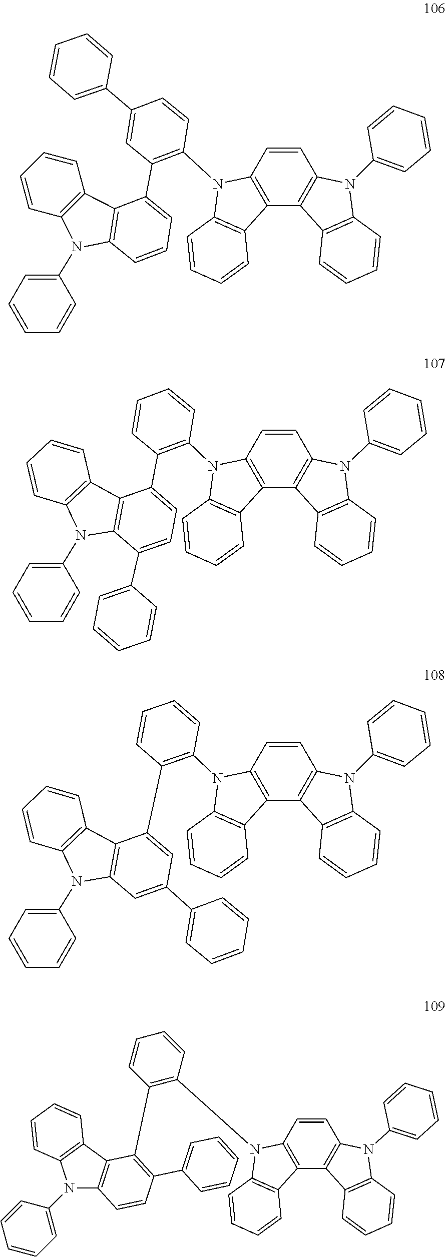 [SMILES: c1ccc(-c2ccc(-n3c4ccccc4c4c5c6ccccc6n(-c6ccccc6)c5ccc43)c(-c3cccc4c3c3ccccc3n4-c3ccccc3)c2)cc1 ; c1ccc(-c2ccc(-c3ccccc3-n3c4ccccc4c4c5c6ccccc6n(-c6ccccc6)c5ccc43)c3c4ccccc4n(-c4ccccc4)c23)cc1 ; c1ccc(-c2cc(-c3ccccc3-n3c4ccccc4c4c5c6ccccc6n(-c6ccccc6)c5ccc43)c3c4ccccc4n(-c4ccccc4)c3c2)cc1 ; c1ccc(-c2ccc3c(c2-c2ccccc2-n2c4ccccc4c4c5c6ccccc6n(-c6ccccc6)c5ccc42)c2ccccc2n3-c2ccccc2)cc1]

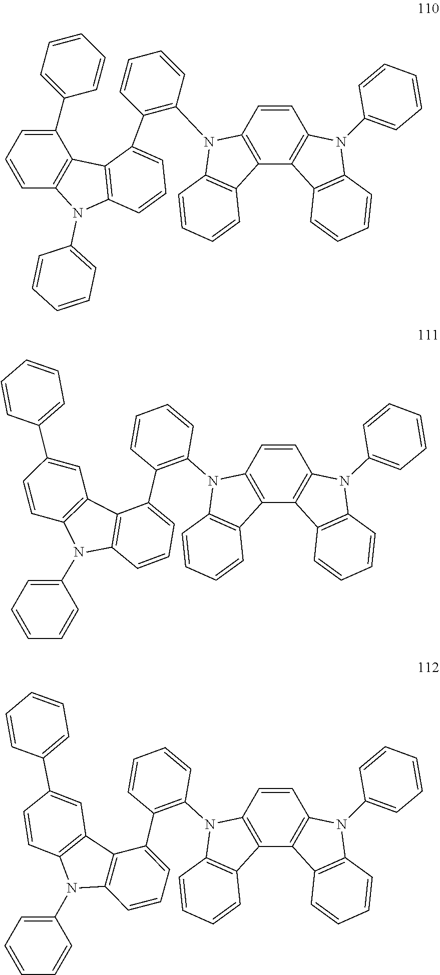 [SMILES: c1ccc(-c2cccc3c2c2c(-c4ccccc4-n4c5ccccc5c5c6c7ccccc7n(-c7ccccc7)c6ccc54)cccc2n3-c2ccccc2)cc1 ; c1ccc(-c2ccc3c(c2)c2c(-c4ccccc4-n4c5ccccc5c5c6c7ccccc7n(-c7ccccc7)c6ccc54)cccc2n3-c2ccccc2)cc1 ; c1ccc(-c2ccc3c(c2)c2c(-c4ccccc4-n4c5ccccc5c5c6c7ccccc7n(-c7ccccc7)c6ccc54)cccc2n3-c2ccccc2)cc1]

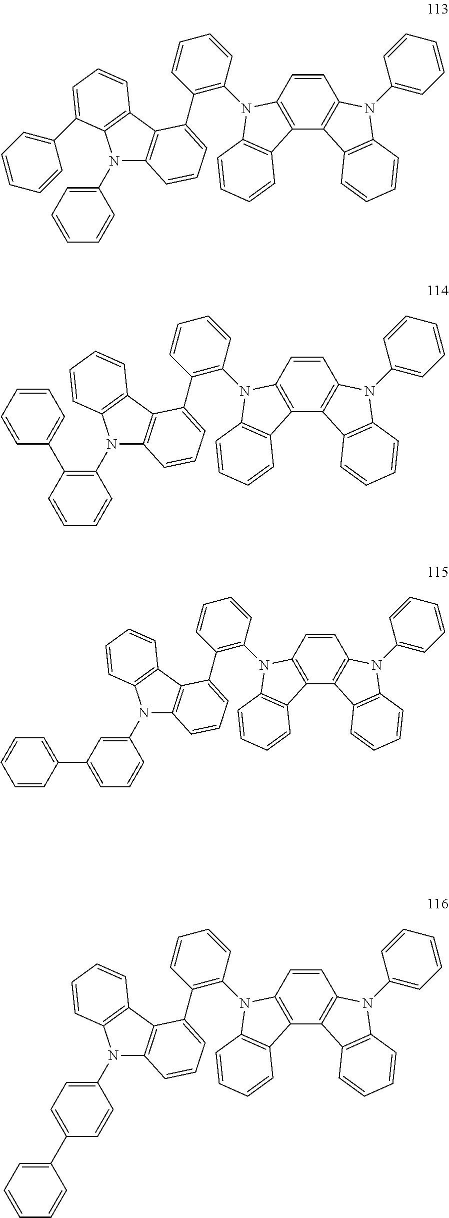 [SMILES: c1ccc(-c2cccc3c4c(-c5ccccc5-n5c6ccccc6c6c7c8ccccc8n(-c8ccccc8)c7ccc65)cccc4n(-c4ccccc4)c23)cc1 ; c1ccc(-c2ccccc2-n2c3ccccc3c3c(-c4ccccc4-n4c5ccccc5c5c6c7ccccc7n(-c7ccccc7)c6ccc54)cccc32)cc1 ; c1ccc(-c2cccc(-n3c4ccccc4c4c(-c5ccccc5-n5c6ccccc6c6c7c8ccccc8n(-c8ccccc8)c7ccc65)cccc43)c2)cc1 ; c1ccc(-c2ccc(-n3c4ccccc4c4c(-c5ccccc5-n5c6ccccc6c6c7c8ccccc8n(-c8ccccc8)c7ccc65)cccc43)cc2)cc1]

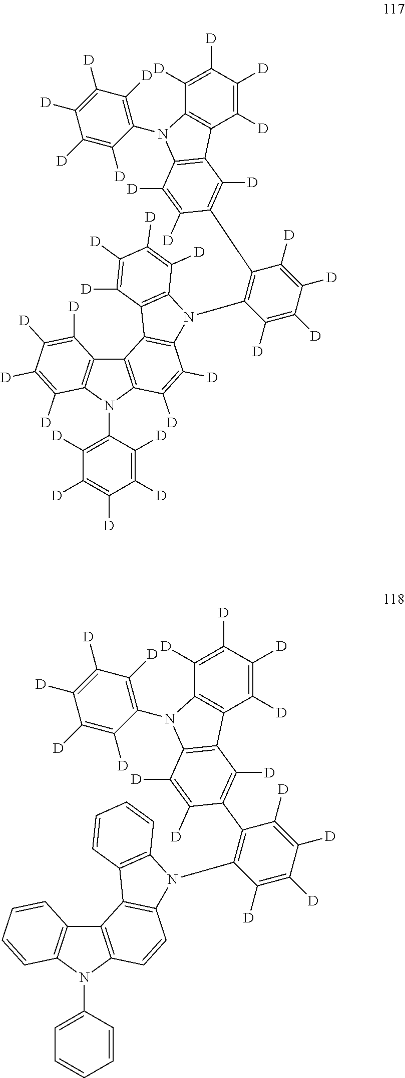 [SMILES: [2H]c1c([2H])c([2H])c(-n2c3c([2H])c([2H])c([2H])c([2H])c3c3c([2H])c(-c4c([2H])c([2H])c([2H])c([2H])c4-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c([2H])c32)c([2H])c1[2H] ; [2H]c1c([2H])c([2H])c(-n2c3c([2H])c([2H])c([2H])c([2H])c3c3c([2H])c(-c4c([2H])c([2H])c([2H])c([2H])c4-n4c5ccccc5c5c6c7ccccc7n(-c7ccccc7)c6ccc54)c([2H])c([2H])c32)c([2H])c1[2H]]

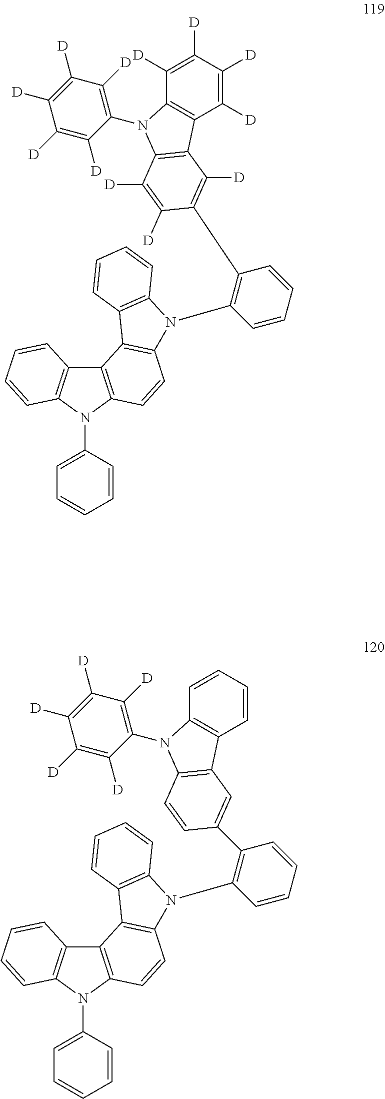 [SMILES: [2H]c1c([2H])c([2H])c(-n2c3c([2H])c([2H])c([2H])c([2H])c3c3c([2H])c(-c4ccccc4-n4c5ccccc5c5c6c7ccccc7n(-c7ccccc7)c6ccc54)c([2H])c([2H])c32)c([2H])c1[2H] ; [2H]c1c([2H])c([2H])c(-n2c3ccccc3c3cc(-c4ccccc4-n4c5ccccc5c5c6c7ccccc7n(-c7ccccc7)c6ccc54)ccc32)c([2H])c1[2H]]

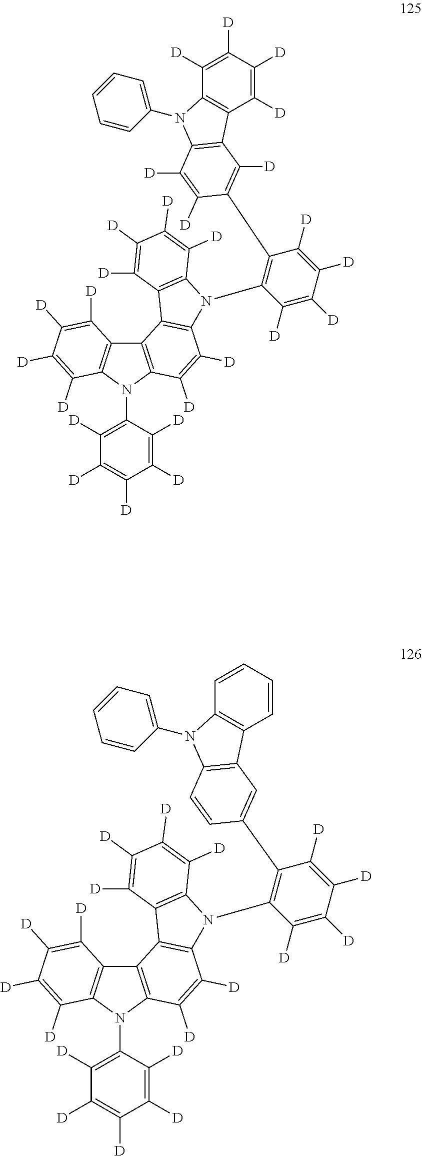 [SMILES: [2H]c1c([2H])c([2H])c(-n2c3c([2H])c([2H])c([2H])c([2H])c3c3c4c5c([2H])c([2H])c([2H])c([2H])c5n(-c5c([2H])c([2H])c([2H])c([2H])c5-c5c([2H])c([2H])c6c(c5[2H])c5c([2H])c([2H])c([2H])c([2H])c5n6-c5ccccc5)c4c([2H])c([2H])c32)c([2H])c1[2H] ; [2H]c1c([2H])c([2H])c(-n2c3c([2H])c([2H])c([2H])c([2H])c3c3c4c5c([2H])c([2H])c([2H])c([2H])c5n(-c5c([2H])c([2H])c([2H])c([2H])c5-c5ccc6c(c5)c5ccccc5n6-c5ccccc5)c4c([2H])c([2H])c32)c([2H])c1[2H]]

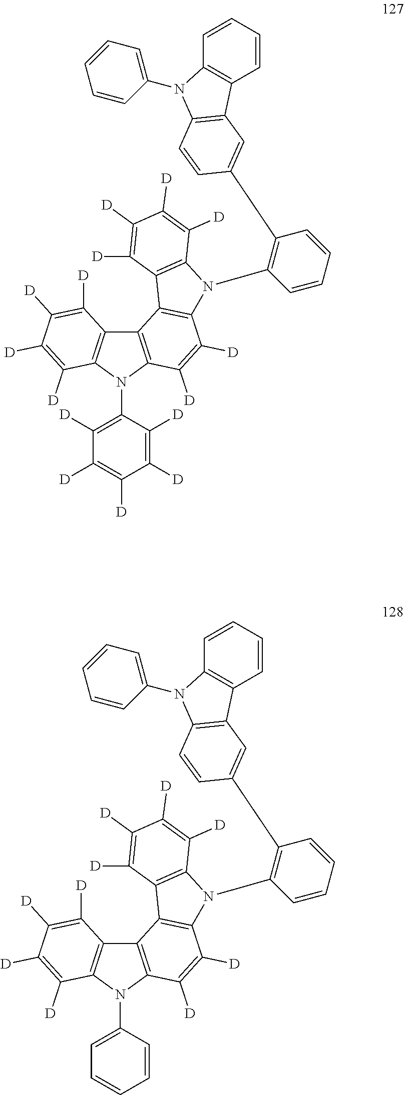 [SMILES: [2H]c1c([2H])c([2H])c(-n2c3c([2H])c([2H])c([2H])c([2H])c3c3c4c5c([2H])c([2H])c([2H])c([2H])c5n(-c5ccccc5-c5ccc6c(c5)c5ccccc5n6-c5ccccc5)c4c([2H])c([2H])c32)c([2H])c1[2H] ; [2H]c1c([2H])c([2H])c2c(c1[2H])c1c3c4c([2H])c([2H])c([2H])c([2H])c4n(-c4ccccc4-c4ccc5c(c4)c4ccccc4n5-c4ccccc4)c3c([2H])c([2H])c1n2-c1ccccc1]

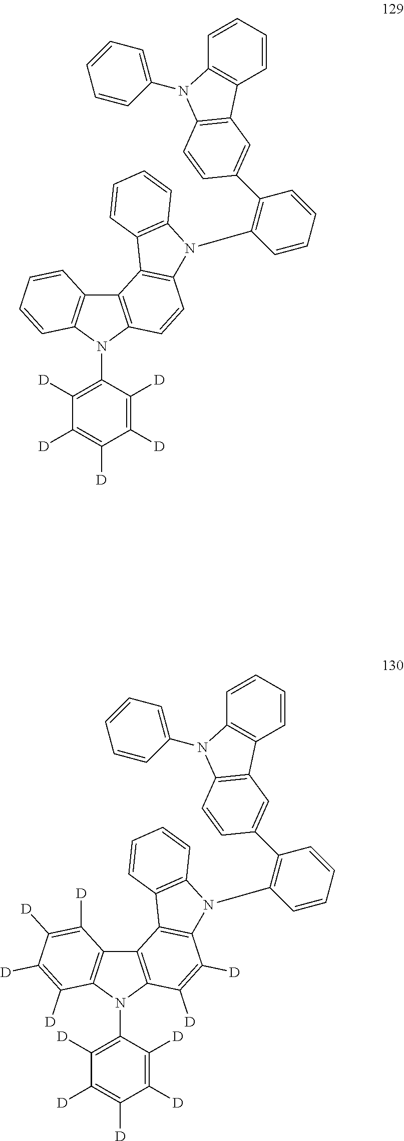 [SMILES: [2H]c1c([2H])c([2H])c(-n2c3ccccc3c3c4c5ccccc5n(-c5ccccc5-c5ccc6c(c5)c5ccccc5n6-c5ccccc5)c4ccc32)c([2H])c1[2H] ; [2H]c1c([2H])c([2H])c(-n2c3c([2H])c([2H])c([2H])c([2H])c3c3c4c5ccccc5n(-c5ccccc5-c5ccc6c(c5)c5ccccc5n6-c5ccccc5)c4c([2H])c([2H])c32)c([2H])c1[2H]]

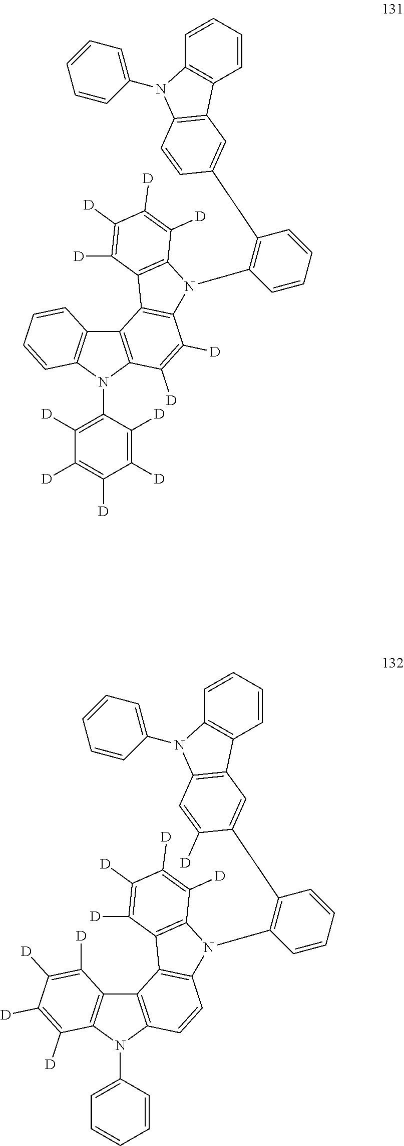 [SMILES: [2H]c1c([2H])c([2H])c(-n2c3ccccc3c3c4c5c([2H])c([2H])c([2H])c([2H])c5n(-c5ccccc5-c5ccc6c(c5)c5ccccc5n6-c5ccccc5)c4c([2H])c([2H])c32)c([2H])c1[2H] ; [2H]c1cc2c(cc1-c1ccccc1-n1c3ccc4c(c5c([2H])c([2H])c([2H])c([2H])c5n4-c4ccccc4)c3c3c([2H])c([2H])c([2H])c([2H])c31)c1ccccc1n2-c1ccccc1]

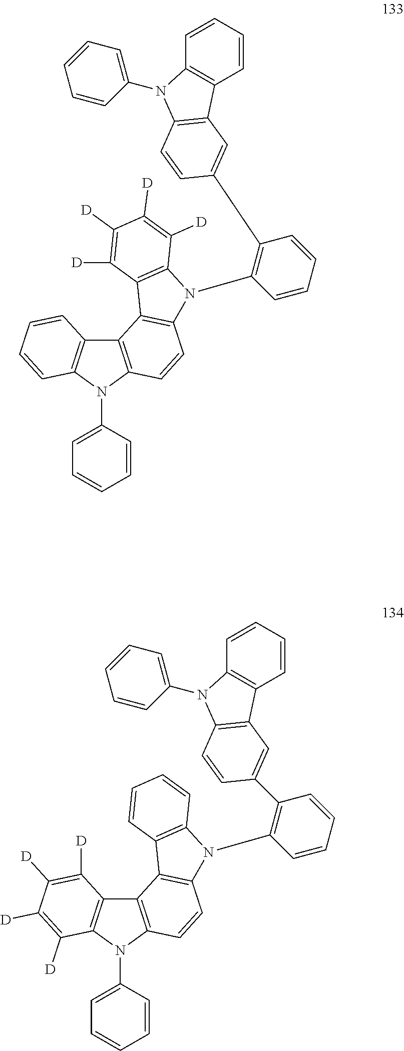 [SMILES: [2H]c1c([2H])c([2H])c2c(c1[2H])c1c3c4ccccc4n(-c4ccccc4)c3ccc1n2-c1ccccc1-c1ccc2c(c1)c1ccccc1n2-c1ccccc1 ; [2H]c1c([2H])c([2H])c2c(c1[2H])c1c3c4ccccc4n(-c4ccccc4-c4ccc5c(c4)c4ccccc4n5-c4ccccc4)c3ccc1n2-c1ccccc1]

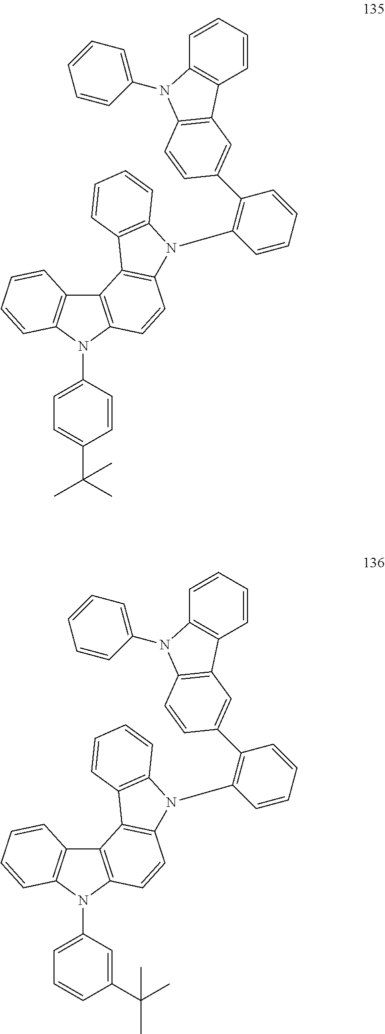 [SMILES: CC(C)(C)c1ccc(-n2c3ccccc3c3c4c5ccccc5n(-c5ccccc5-c5ccc6c(c5)c5ccccc5n6-c5ccccc5)c4ccc32)cc1 ; CC(C)(C)c1cccc(-n2c3ccccc3c3c4c5ccccc5n(-c5ccccc5-c5ccc6c(c5)c5ccccc5n6-c5ccccc5)c4ccc32)c1]

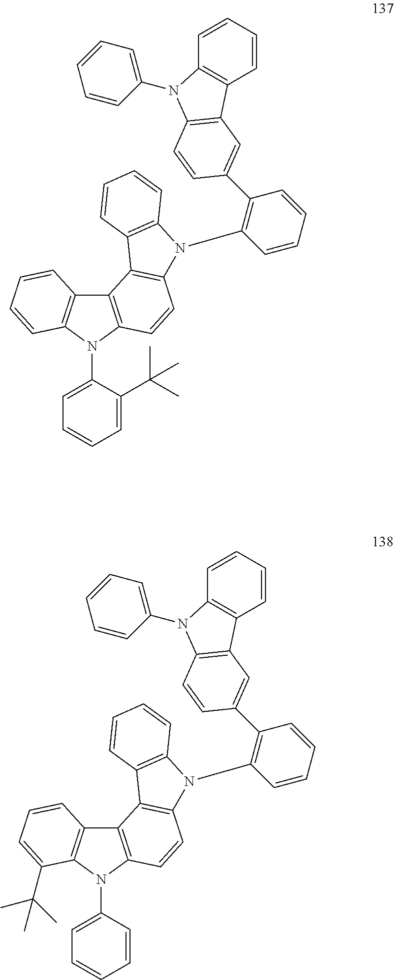 [SMILES: CC(C)(C)c1ccccc1-n1c2ccccc2c2c3c4ccccc4n(-c4ccccc4-c4ccc5c(c4)c4ccccc4n5-c4ccccc4)c3ccc21 ; CC(C)(C)c1cccc2c3c4c5ccccc5n(-c5ccccc5-c5ccc6c(c5)c5ccccc5n6-c5ccccc5)c4ccc3n(-c3ccccc3)c12]

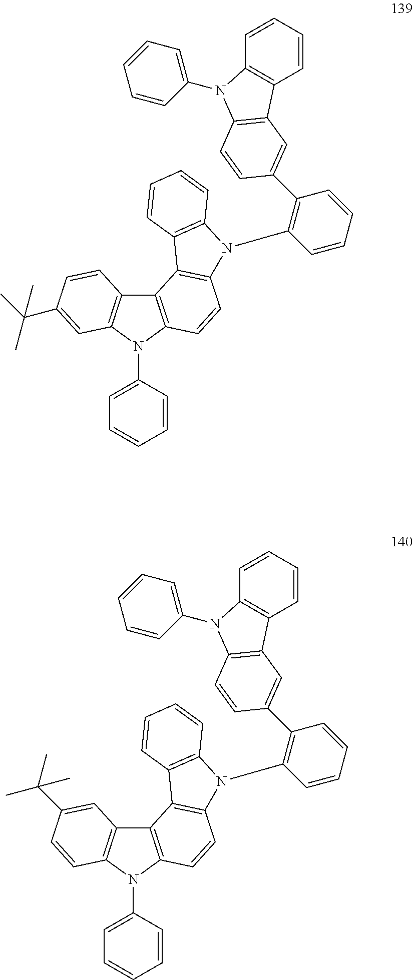 [SMILES: CC(C)(C)c1ccc2c3c4c5ccccc5n(-c5ccccc5-c5ccc6c(c5)c5ccccc5n6-c5ccccc5)c4ccc3n(-c3ccccc3)c2c1 ; CC(C)(C)c1ccc2c(c1)c1c3c4ccccc4n(-c4ccccc4-c4ccc5c(c4)c4ccccc4n5-c4ccccc4)c3ccc1n2-c1ccccc1]

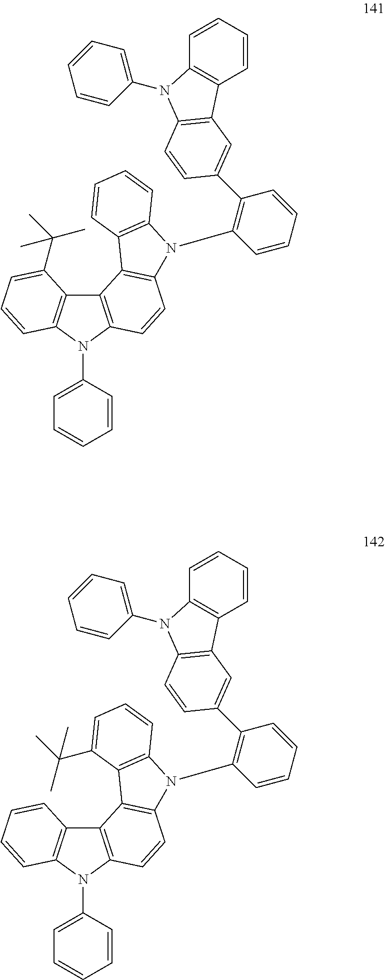 [SMILES: CC(C)(C)c1cccc2c1c1c3c4ccccc4n(-c4ccccc4-c4ccc5c(c4)c4ccccc4n5-c4ccccc4)c3ccc1n2-c1ccccc1 ; CC(C)(C)c1cccc2c1c1c3c4ccccc4n(-c4ccccc4)c3ccc1n2-c1ccccc1-c1ccc2c(c1)c1ccccc1n2-c1ccccc1]

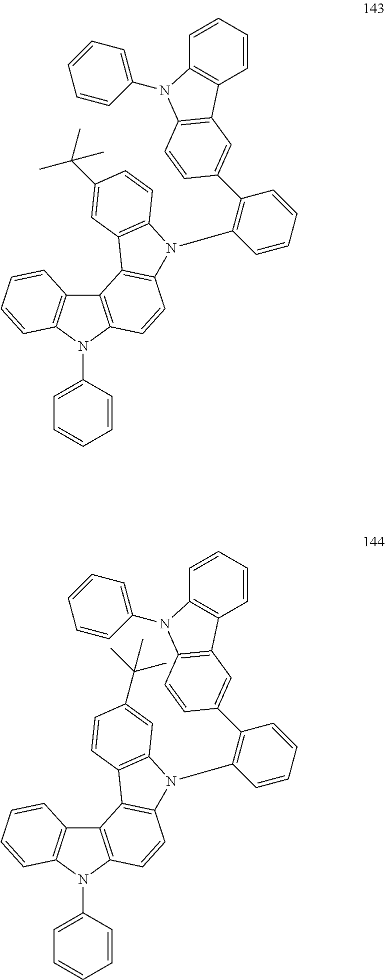 [SMILES: CC(C)(C)c1ccc2c(c1)c1c3c4ccccc4n(-c4ccccc4)c3ccc1n2-c1ccccc1-c1ccc2c(c1)c1ccccc1n2-c1ccccc1 ; CC(C)(C)c1ccc2c3c4c5ccccc5n(-c5ccccc5)c4ccc3n(-c3ccccc3-c3ccc4c(c3)c3ccccc3n4-c3ccccc3)c2c1]

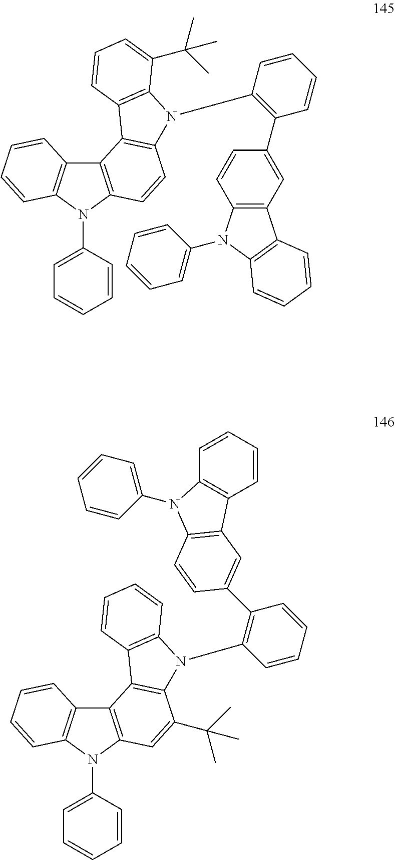 [SMILES: CC(C)(C)c1cccc2c3c4c5ccccc5n(-c5ccccc5)c4ccc3n(-c3ccccc3-c3ccc4c(c3)c3ccccc3n4-c3ccccc3)c12 ; CC(C)(C)c1cc2c(c3ccccc3n2-c2ccccc2)c2c3ccccc3n(-c3ccccc3-c3ccc4c(c3)c3ccccc3n4-c3ccccc3)c12]

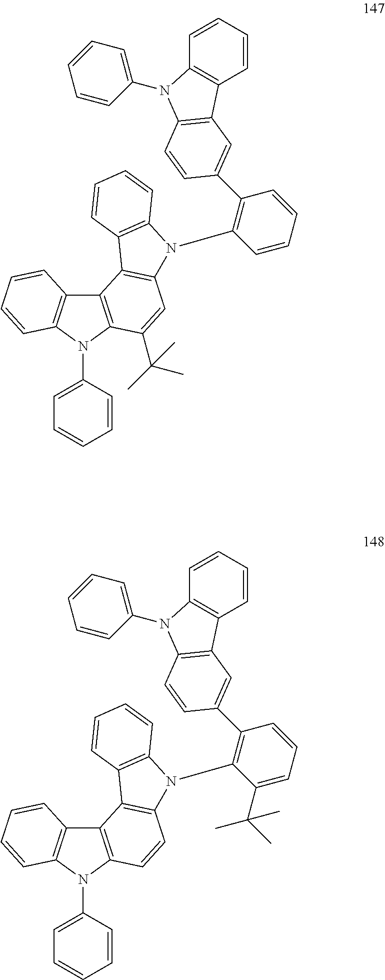 [SMILES: CC(C)(C)c1cc2c(c3ccccc3n2-c2ccccc2-c2ccc3c(c2)c2ccccc2n3-c2ccccc2)c2c3ccccc3n(-c3ccccc3)c12 ; CC(C)(C)c1cccc(-c2ccc3c(c2)c2ccccc2n3-c2ccccc2)c1-n1c2ccccc2c2c3c4ccccc4n(-c4ccccc4)c3ccc21]

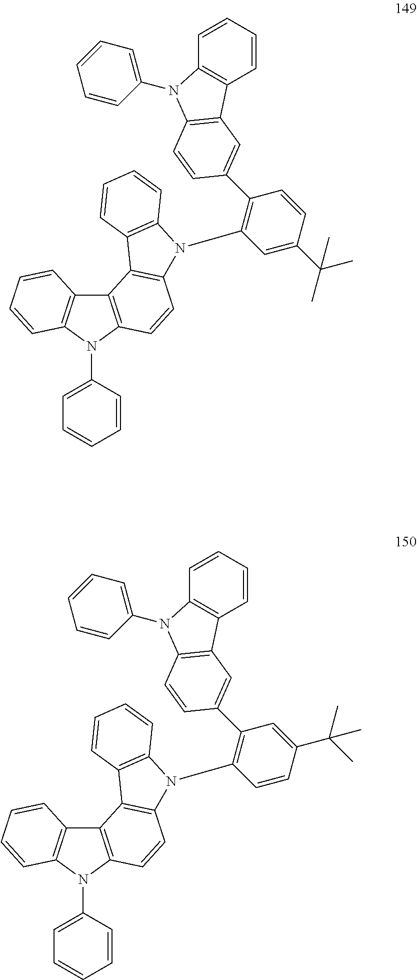 [SMILES: CC(C)(C)c1ccc(-c2ccc3c(c2)c2ccccc2n3-c2ccccc2)c(-n2c3ccccc3c3c4c5ccccc5n(-c5ccccc5)c4ccc32)c1 ; CC(C)(C)c1ccc(-n2c3ccccc3c3c4c5ccccc5n(-c5ccccc5)c4ccc32)c(-c2ccc3c(c2)c2ccccc2n3-c2ccccc2)c1]

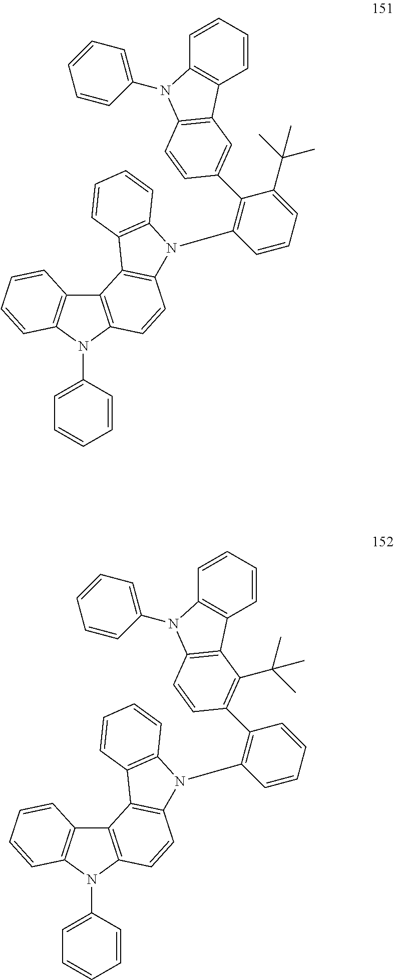 [SMILES: CC(C)(C)c1cccc(-n2c3ccccc3c3c4c5ccccc5n(-c5ccccc5)c4ccc32)c1-c1ccc2c(c1)c1ccccc1n2-c1ccccc1 ; CC(C)(C)c1c(-c2ccccc2-n2c3ccccc3c3c4c5ccccc5n(-c5ccccc5)c4ccc32)ccc2c1c1ccccc1n2-c1ccccc1]

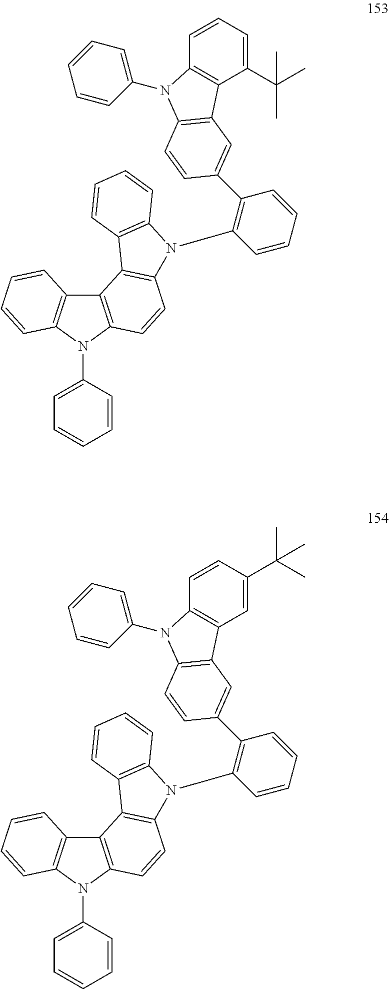 [SMILES: CC(C)(C)c1cccc2c1c1cc(-c3ccccc3-n3c4ccccc4c4c5c6ccccc6n(-c6ccccc6)c5ccc43)ccc1n2-c1ccccc1 ; CC(C)(C)c1ccc2c(c1)c1cc(-c3ccccc3-n3c4ccccc4c4c5c6ccccc6n(-c6ccccc6)c5ccc43)ccc1n2-c1ccccc1]

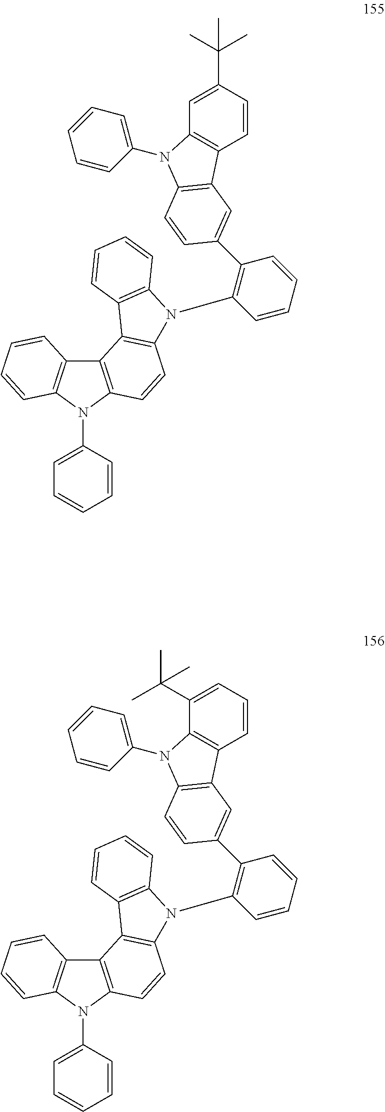 [SMILES: CC(C)(C)c1ccc2c3cc(-c4ccccc4-n4c5ccccc5c5c6c7ccccc7n(-c7ccccc7)c6ccc54)ccc3n(-c3ccccc3)c2c1 ; CC(C)(C)c1cccc2c3cc(-c4ccccc4-n4c5ccccc5c5c6c7ccccc7n(-c7ccccc7)c6ccc54)ccc3n(-c3ccccc3)c12]

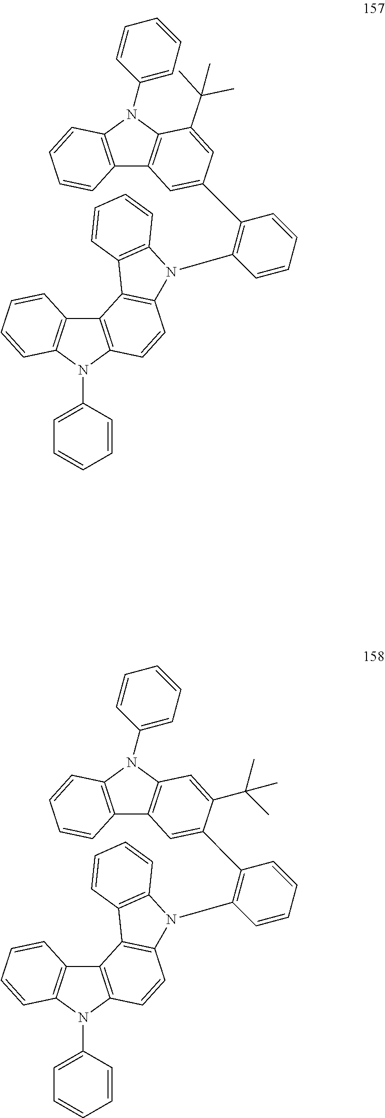 [SMILES: CC(C)(C)c1cc(-c2ccccc2-n2c3ccccc3c3c4c5ccccc5n(-c5ccccc5)c4ccc32)cc2c3ccccc3n(-c3ccccc3)c12 ; CC(C)(C)c1cc2c(cc1-c1ccccc1-n1c3ccccc3c3c4c5ccccc5n(-c5ccccc5)c4ccc31)c1ccccc1n2-c1ccccc1]

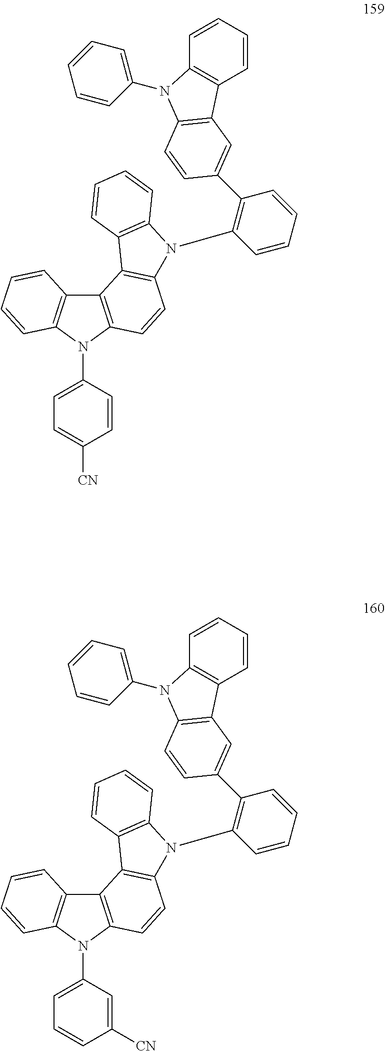 [SMILES: N#Cc1ccc(-n2c3ccccc3c3c4c5ccccc5n(-c5ccccc5-c5ccc6c(c5)c5ccccc5n6-c5ccccc5)c4ccc32)cc1 ; N#Cc1cccc(-n2c3ccccc3c3c4c5ccccc5n(-c5ccccc5-c5ccc6c(c5)c5ccccc5n6-c5ccccc5)c4ccc32)c1]

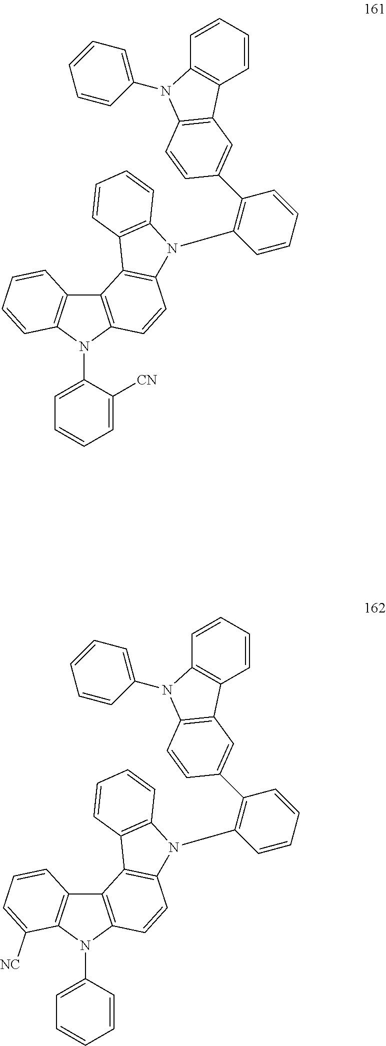 [SMILES: N#Cc1ccccc1-n1c2ccccc2c2c3c4ccccc4n(-c4ccccc4-c4ccc5c(c4)c4ccccc4n5-c4ccccc4)c3ccc21 ; N#Cc1cccc2c3c4c5ccccc5n(-c5ccccc5-c5ccc6c(c5)c5ccccc5n6-c5ccccc5)c4ccc3n(-c3ccccc3)c12]

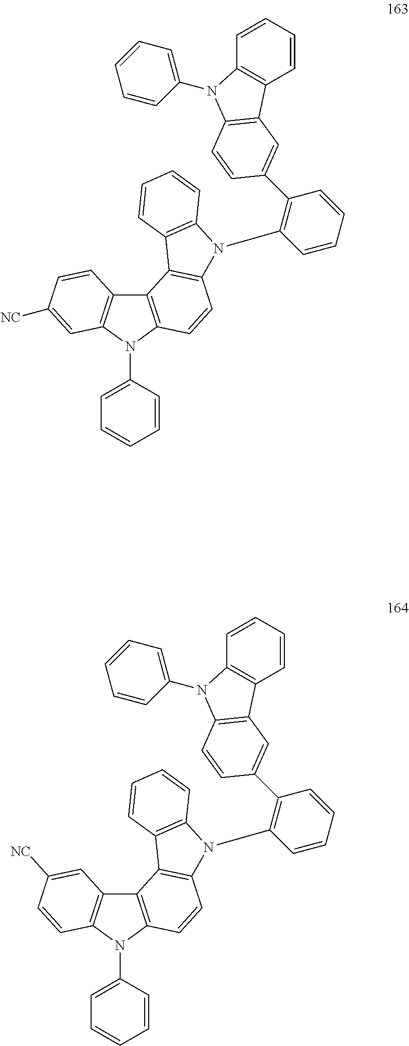 [SMILES: N#Cc1ccc2c3c4c5ccccc5n(-c5ccccc5-c5ccc6c(c5)c5ccccc5n6-c5ccccc5)c4ccc3n(-c3ccccc3)c2c1 ; N#Cc1ccc2c(c1)c1c3c4ccccc4n(-c4ccccc4-c4ccc5c(c4)c4ccccc4n5-c4ccccc4)c3ccc1n2-c1ccccc1]

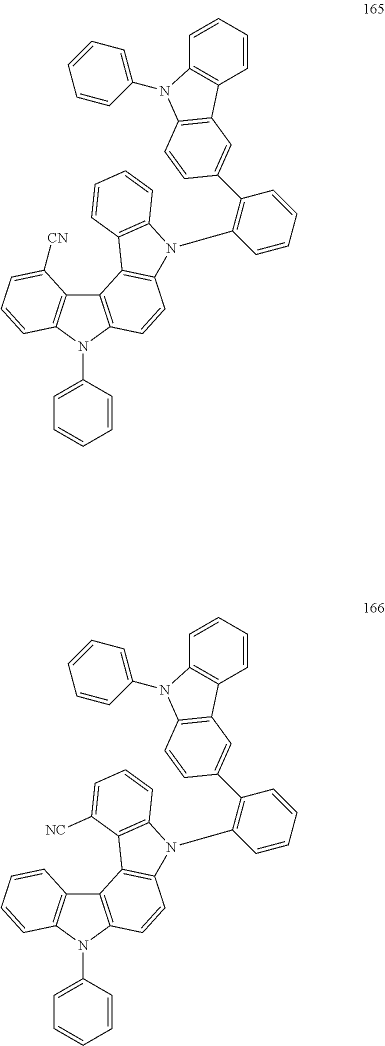 [SMILES: N#Cc1cccc2c1c1c3c4ccccc4n(-c4ccccc4-c4ccc5c(c4)c4ccccc4n5-c4ccccc4)c3ccc1n2-c1ccccc1 ; N#Cc1cccc2c1c1c3c4ccccc4n(-c4ccccc4)c3ccc1n2-c1ccccc1-c1ccc2c(c1)c1ccccc1n2-c1ccccc1]

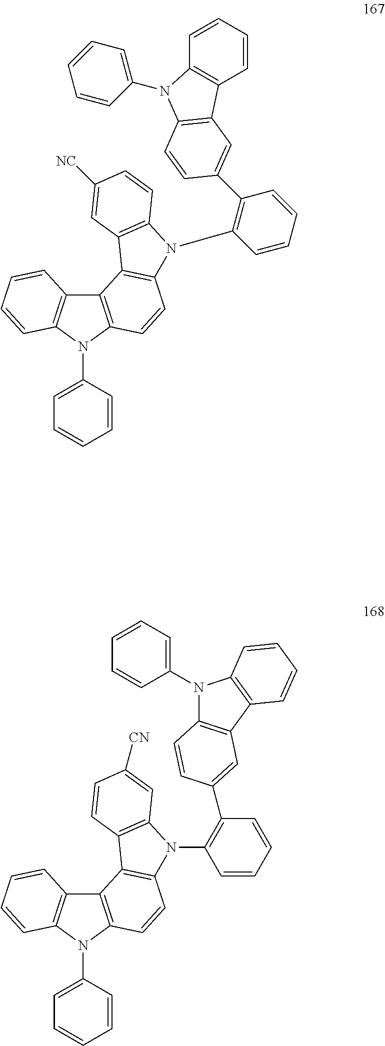 [SMILES: N#Cc1ccc2c(c1)c1c3c4ccccc4n(-c4ccccc4)c3ccc1n2-c1ccccc1-c1ccc2c(c1)c1ccccc1n2-c1ccccc1 ; N#Cc1ccc2c3c4c5ccccc5n(-c5ccccc5)c4ccc3n(-c3ccccc3-c3ccc4c(c3)c3ccccc3n4-c3ccccc3)c2c1]

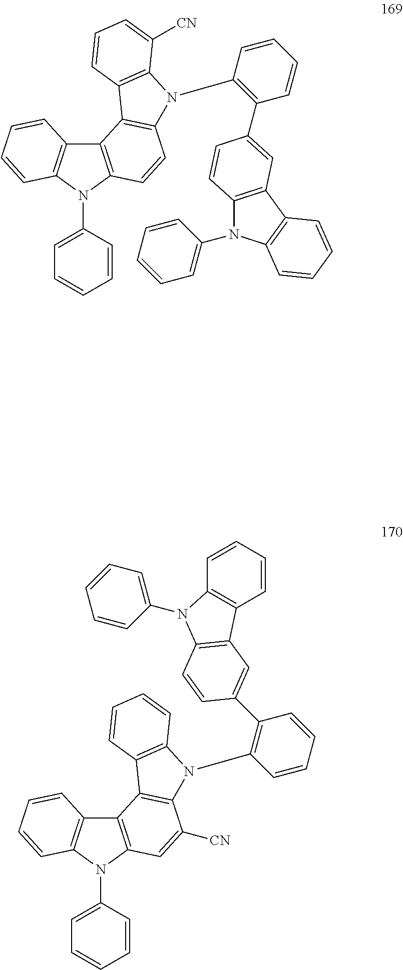 [SMILES: N#Cc1cccc2c3c4c5ccccc5n(-c5ccccc5)c4ccc3n(-c3ccccc3-c3ccc4c(c3)c3ccccc3n4-c3ccccc3)c12 ; N#Cc1cc2c(c3ccccc3n2-c2ccccc2)c2c3ccccc3n(-c3ccccc3-c3ccc4c(c3)c3ccccc3n4-c3ccccc3)c12]

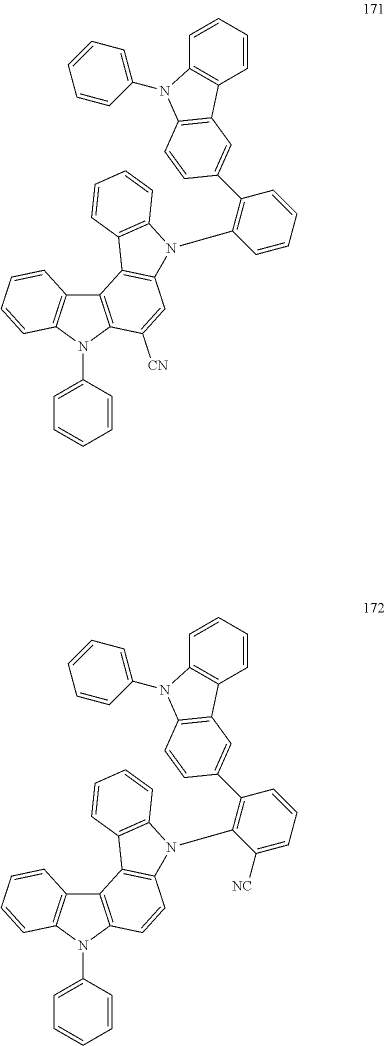 [SMILES: N#Cc1cc2c(c3ccccc3n2-c2ccccc2-c2ccc3c(c2)c2ccccc2n3-c2ccccc2)c2c3ccccc3n(-c3ccccc3)c12 ; N#Cc1cccc(-c2ccc3c(c2)c2ccccc2n3-c2ccccc2)c1-n1c2ccccc2c2c3c4ccccc4n(-c4ccccc4)c3ccc21]

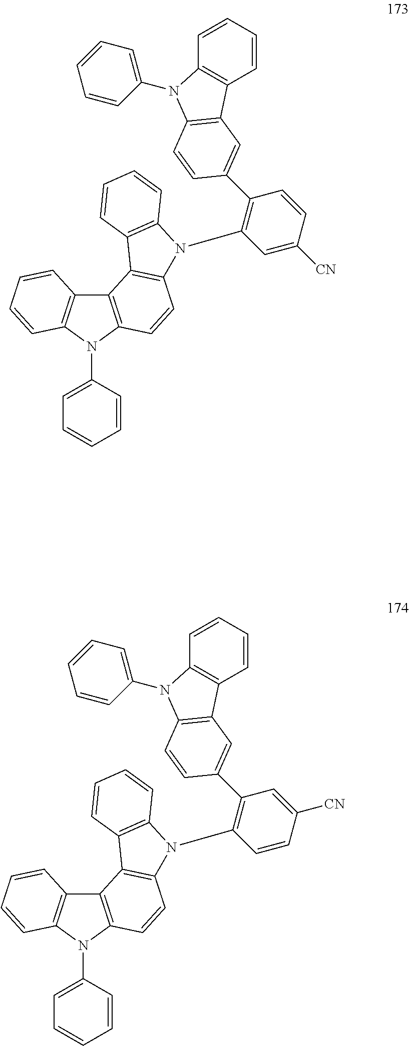 [SMILES: N#Cc1ccc(-c2ccc3c(c2)c2ccccc2n3-c2ccccc2)c(-n2c3ccccc3c3c4c5ccccc5n(-c5ccccc5)c4ccc32)c1 ; N#Cc1ccc(-n2c3ccccc3c3c4c5ccccc5n(-c5ccccc5)c4ccc32)c(-c2ccc3c(c2)c2ccccc2n3-c2ccccc2)c1]

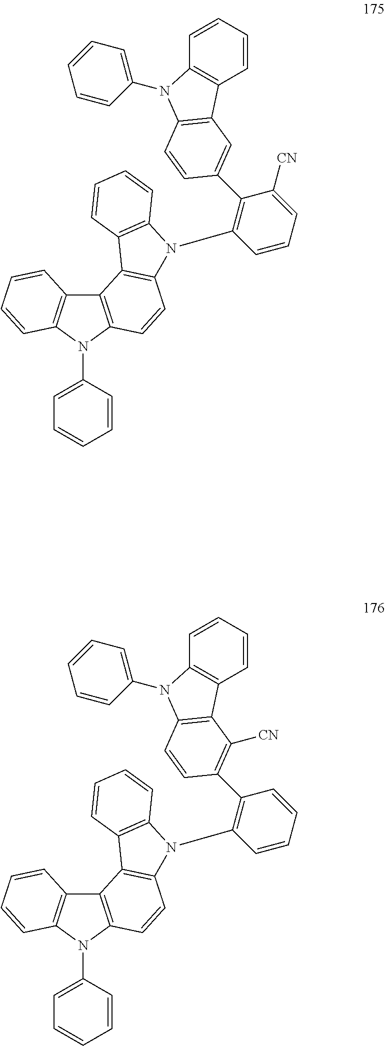 [SMILES: N#Cc1cccc(-n2c3ccccc3c3c4c5ccccc5n(-c5ccccc5)c4ccc32)c1-c1ccc2c(c1)c1ccccc1n2-c1ccccc1 ; N#Cc1c(-c2ccccc2-n2c3ccccc3c3c4c5ccccc5n(-c5ccccc5)c4ccc32)ccc2c1c1ccccc1n2-c1ccccc1]

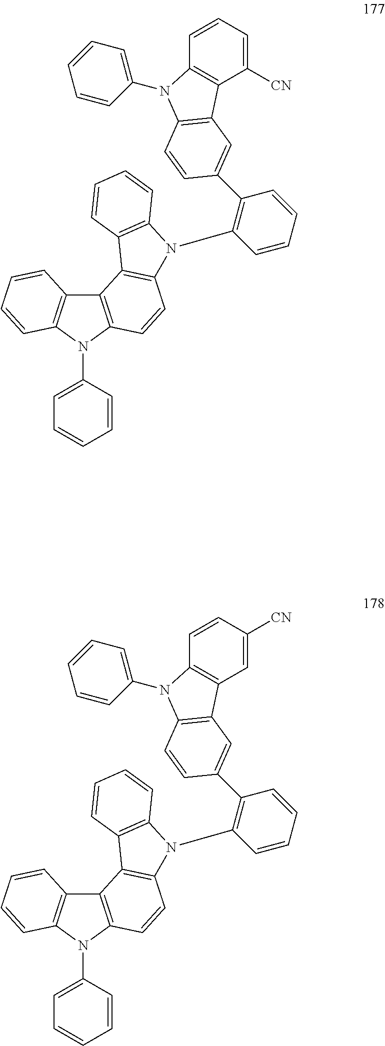 [SMILES: N#Cc1cccc2c1c1cc(-c3ccccc3-n3c4ccccc4c4c5c6ccccc6n(-c6ccccc6)c5ccc43)ccc1n2-c1ccccc1 ; N#Cc1ccc2c(c1)c1cc(-c3ccccc3-n3c4ccccc4c4c5c6ccccc6n(-c6ccccc6)c5ccc43)ccc1n2-c1ccccc1]

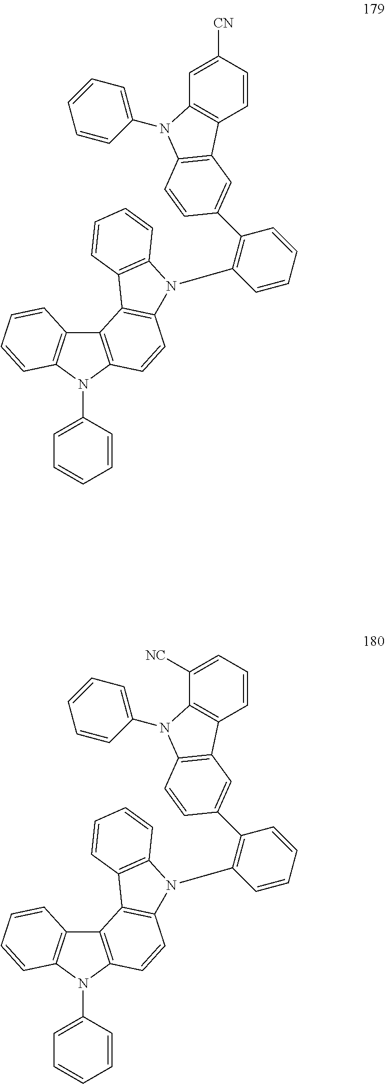 [SMILES: N#Cc1ccc2c3cc(-c4ccccc4-n4c5ccccc5c5c6c7ccccc7n(-c7ccccc7)c6ccc54)ccc3n(-c3ccccc3)c2c1 ; N#Cc1cccc2c3cc(-c4ccccc4-n4c5ccccc5c5c6c7ccccc7n(-c7ccccc7)c6ccc54)ccc3n(-c3ccccc3)c12]

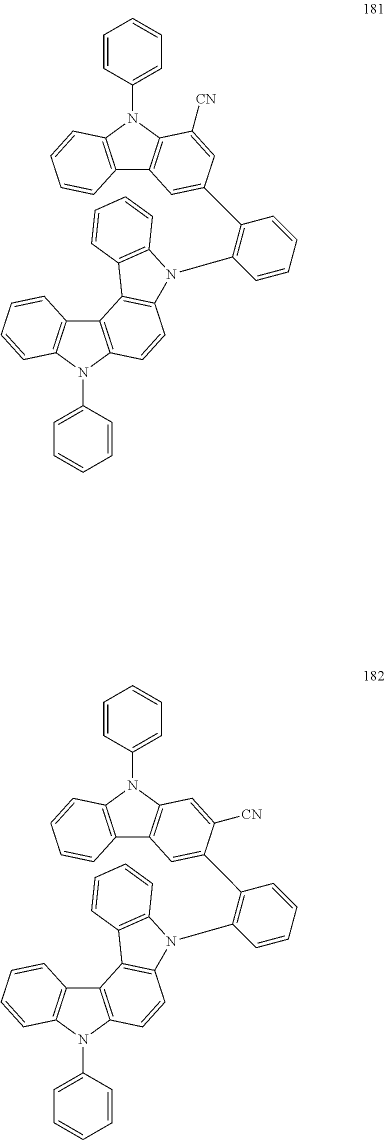 [SMILES: N#Cc1cc(-c2ccccc2-n2c3ccccc3c3c4c5ccccc5n(-c5ccccc5)c4ccc32)cc2c3ccccc3n(-c3ccccc3)c12 ; N#Cc1cc2c(cc1-c1ccccc1-n1c3ccccc3c3c4c5ccccc5n(-c5ccccc5)c4ccc31)c1ccccc1n2-c1ccccc1]

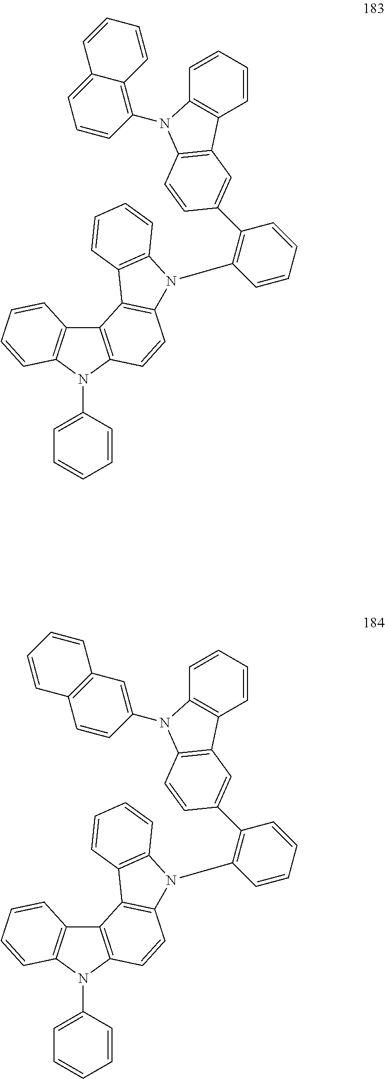 [SMILES: c1ccc(-n2c3ccccc3c3c4c5ccccc5n(-c5ccccc5-c5ccc6c(c5)c5ccccc5n6-c5cccc6ccccc56)c4ccc32)cc1 ; c1ccc(-n2c3ccccc3c3c4c5ccccc5n(-c5ccccc5-c5ccc6c(c5)c5ccccc5n6-c5ccc6ccccc6c5)c4ccc32)cc1]

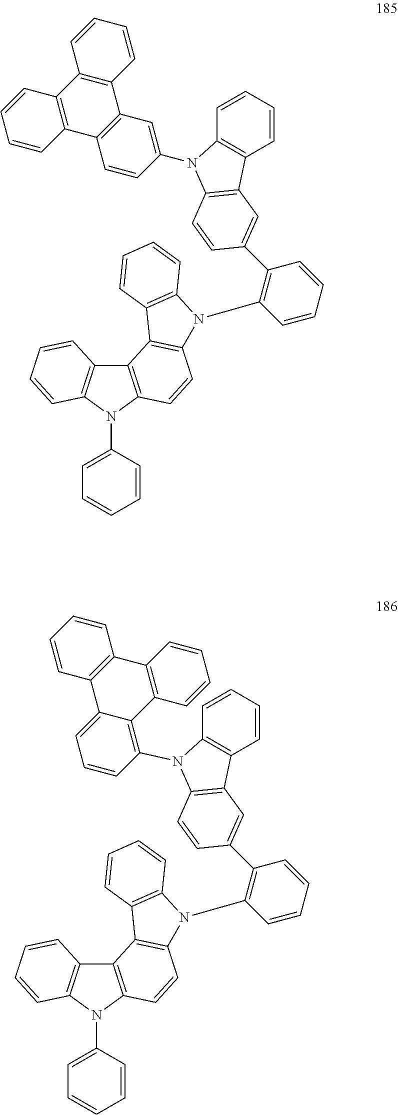 [SMILES: c1ccc(-n2c3ccccc3c3c4c5ccccc5n(-c5ccccc5-c5ccc6c(c5)c5ccccc5n6-c5ccc6c7ccccc7c7ccccc7c6c5)c4ccc32)cc1 ; c1ccc(-n2c3ccccc3c3c4c5ccccc5n(-c5ccccc5-c5ccc6c(c5)c5ccccc5n6-c5cccc6c7ccccc7c7ccccc7c56)c4ccc32)cc1]

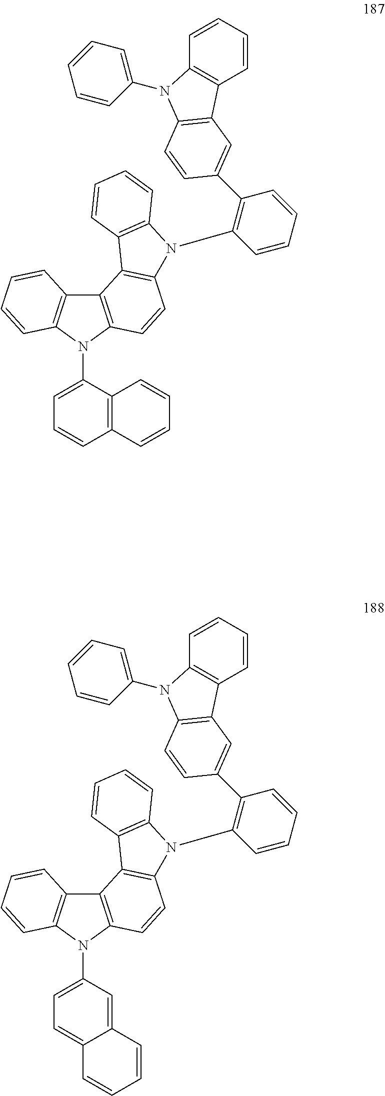 [SMILES: c1ccc(-n2c3ccccc3c3cc(-c4ccccc4-n4c5ccccc5c5c6c7ccccc7n(-c7cccc8ccccc78)c6ccc54)ccc32)cc1 ; c1ccc(-n2c3ccccc3c3cc(-c4ccccc4-n4c5ccccc5c5c6c7ccccc7n(-c7ccc8ccccc8c7)c6ccc54)ccc32)cc1]

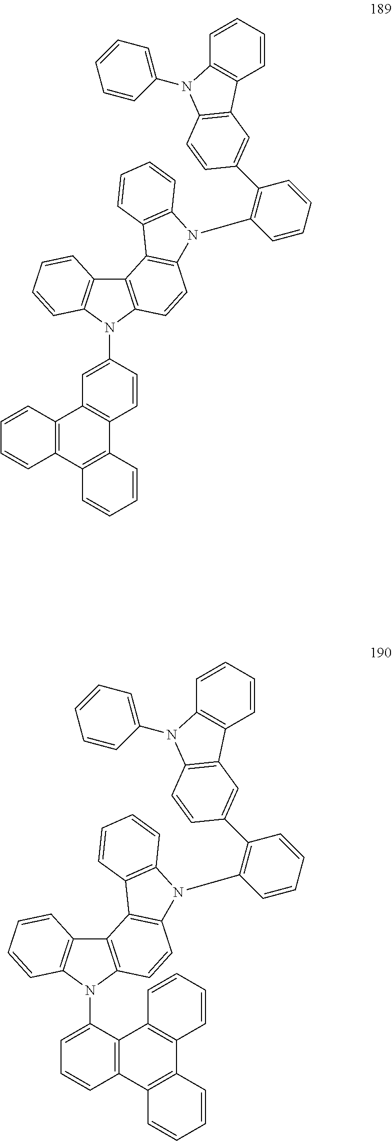 [SMILES: c1ccc(-n2c3ccccc3c3cc(-c4ccccc4-n4c5ccccc5c5c6c7ccccc7n(-c7ccc8c9ccccc9c9ccccc9c8c7)c6ccc54)ccc32)cc1 ; c1ccc(-n2c3ccccc3c3cc(-c4ccccc4-n4c5ccccc5c5c6c7ccccc7n(-c7cccc8c9ccccc9c9ccccc9c78)c6ccc54)ccc32)cc1]

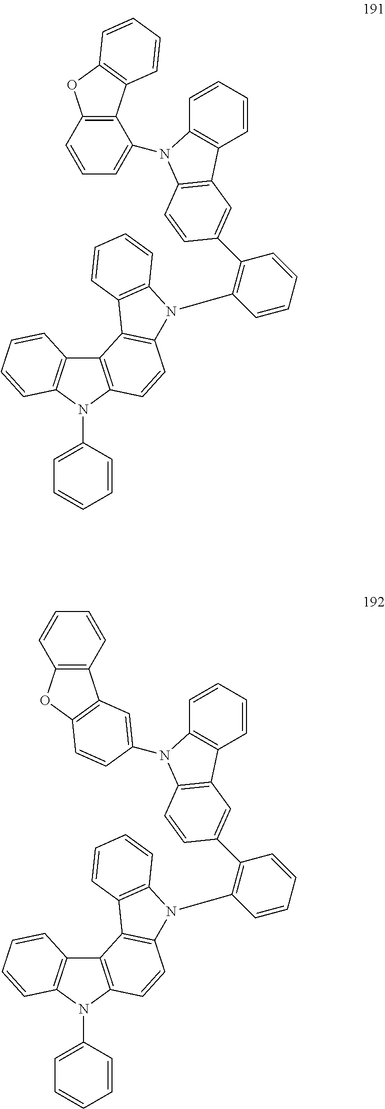 [SMILES: c1ccc(-n2c3ccccc3c3c4c5ccccc5n(-c5ccccc5-c5ccc6c(c5)c5ccccc5n6-c5cccc6oc7ccccc7c56)c4ccc32)cc1 ; c1ccc(-n2c3ccccc3c3c4c5ccccc5n(-c5ccccc5-c5ccc6c(c5)c5ccccc5n6-c5ccc6oc7ccccc7c6c5)c4ccc32)cc1]

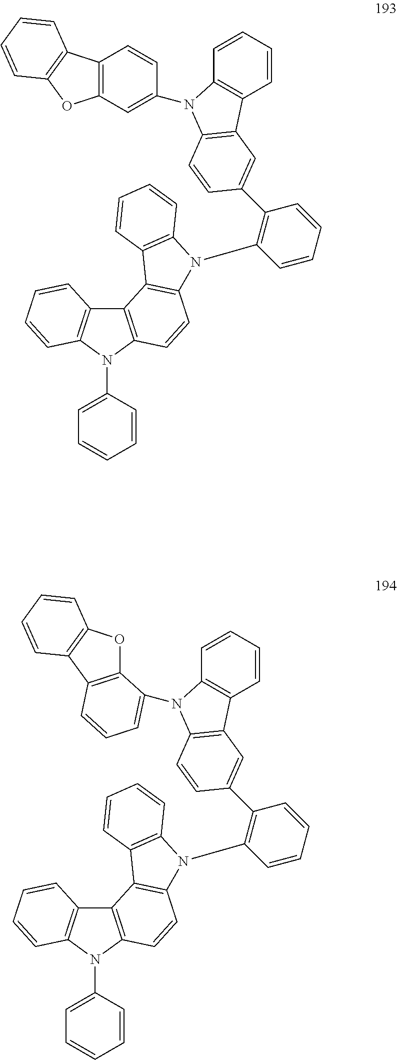 [SMILES: c1ccc(-n2c3ccccc3c3c4c5ccccc5n(-c5ccccc5-c5ccc6c(c5)c5ccccc5n6-c5ccc6c(c5)oc5ccccc56)c4ccc32)cc1 ; c1ccc(-n2c3ccccc3c3c4c5ccccc5n(-c5ccccc5-c5ccc6c(c5)c5ccccc5n6-c5cccc6c5oc5ccccc56)c4ccc32)cc1]

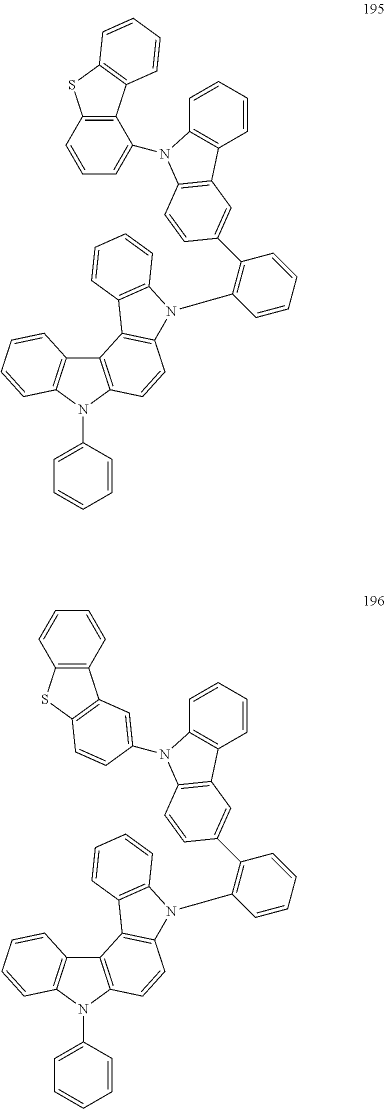 [SMILES: c1ccc(-n2c3ccccc3c3c4c5ccccc5n(-c5ccccc5-c5ccc6c(c5)c5ccccc5n6-c5cccc6sc7ccccc7c56)c4ccc32)cc1 ; c1ccc(-n2c3ccccc3c3c4c5ccccc5n(-c5ccccc5-c5ccc6c(c5)c5ccccc5n6-c5ccc6sc7ccccc7c6c5)c4ccc32)cc1]

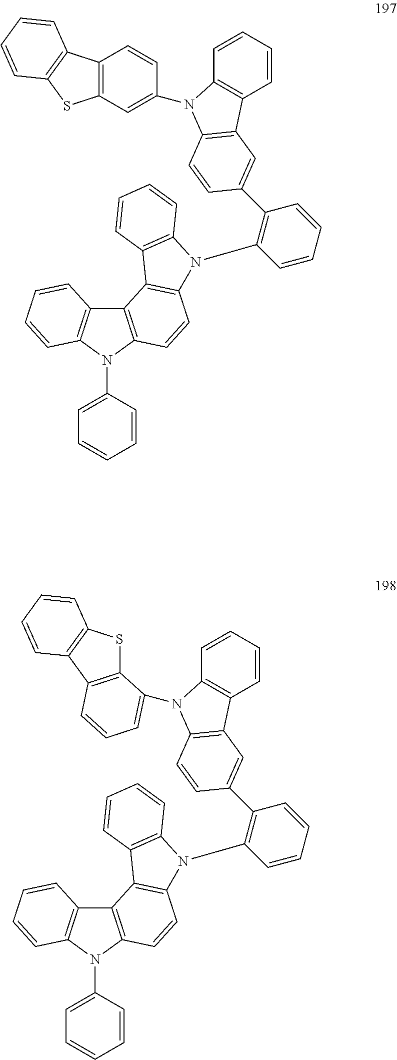 [SMILES: c1ccc(-n2c3ccccc3c3c4c5ccccc5n(-c5ccccc5-c5ccc6c(c5)c5ccccc5n6-c5ccc6c(c5)sc5ccccc56)c4ccc32)cc1 ; c1ccc(-n2c3ccccc3c3c4c5ccccc5n(-c5ccccc5-c5ccc6c(c5)c5ccccc5n6-c5cccc6c5sc5ccccc56)c4ccc32)cc1]

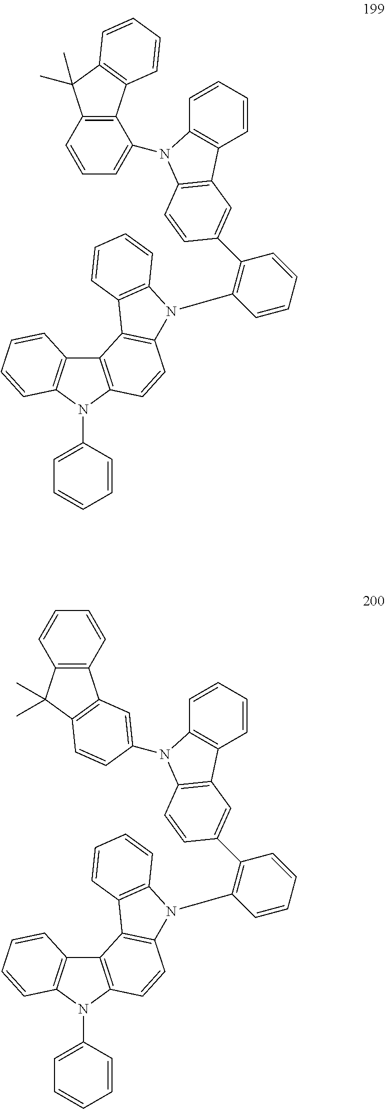 [SMILES: CC1(C)c2ccccc2-c2c(-n3c4ccccc4c4cc(-c5ccccc5-n5c6ccccc6c6c7c8ccccc8n(-c8ccccc8)c7ccc65)ccc43)cccc21 ; CC1(C)c2ccccc2-c2cc(-n3c4ccccc4c4cc(-c5ccccc5-n5c6ccccc6c6c7c8ccccc8n(-c8ccccc8)c7ccc65)ccc43)ccc21]

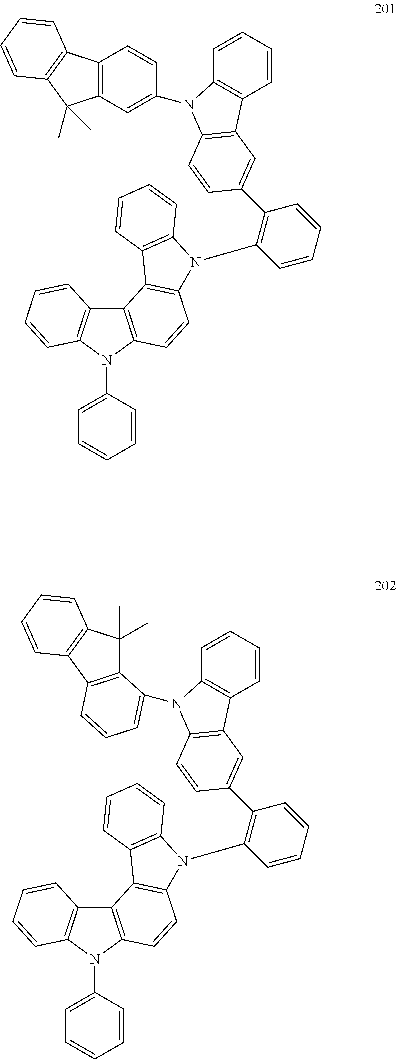 [SMILES: CC1(C)c2ccccc2-c2ccc(-n3c4ccccc4c4cc(-c5ccccc5-n5c6ccccc6c6c7c8ccccc8n(-c8ccccc8)c7ccc65)ccc43)cc21 ; CC1(C)c2ccccc2-c2cccc(-n3c4ccccc4c4cc(-c5ccccc5-n5c6ccccc6c6c7c8ccccc8n(-c8ccccc8)c7ccc65)ccc43)c21]

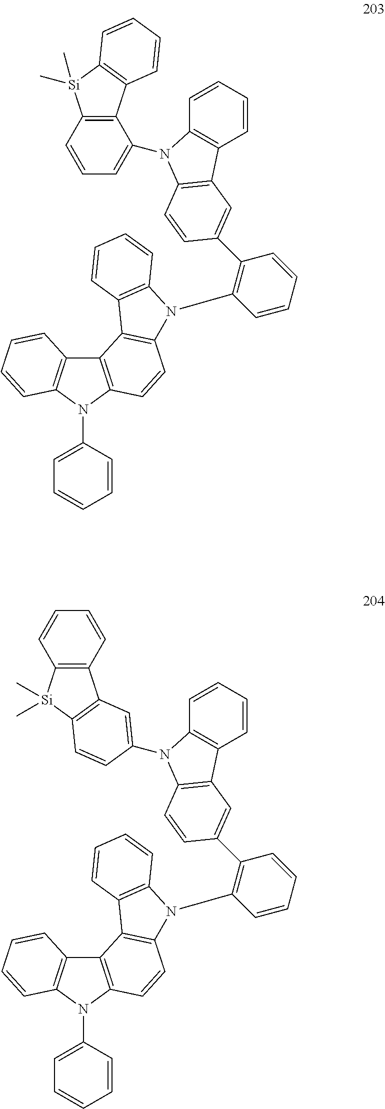 [SMILES: C[Si]1(C)c2ccccc2-c2c(-n3c4ccccc4c4cc(-c5ccccc5-n5c6ccccc6c6c7c8ccccc8n(-c8ccccc8)c7ccc65)ccc43)cccc21 ; C[Si]1(C)c2ccccc2-c2cc(-n3c4ccccc4c4cc(-c5ccccc5-n5c6ccccc6c6c7c8ccccc8n(-c8ccccc8)c7ccc65)ccc43)ccc21]

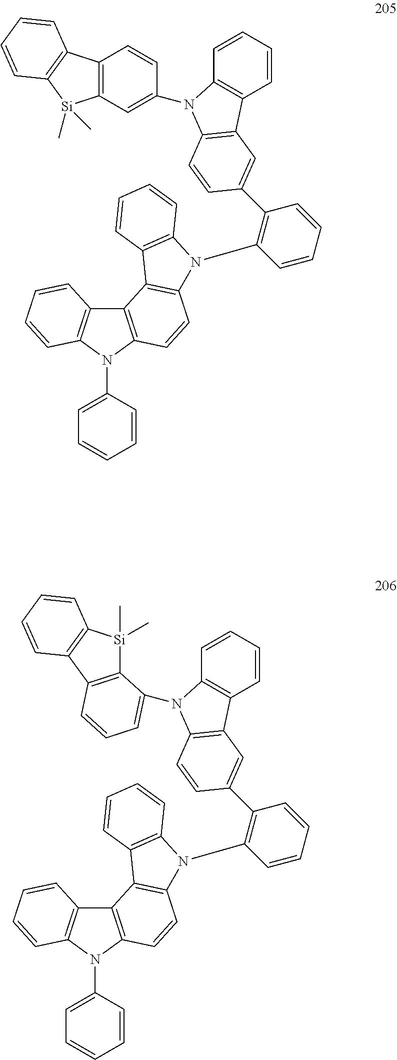 [SMILES: C[Si]1(C)c2ccccc2-c2ccc(-n3c4ccccc4c4cc(-c5ccccc5-n5c6ccccc6c6c7c8ccccc8n(-c8ccccc8)c7ccc65)ccc43)cc21 ; C[Si]1(C)c2ccccc2-c2cccc(-n3c4ccccc4c4cc(-c5ccccc5-n5c6ccccc6c6c7c8ccccc8n(-c8ccccc8)c7ccc65)ccc43)c21]

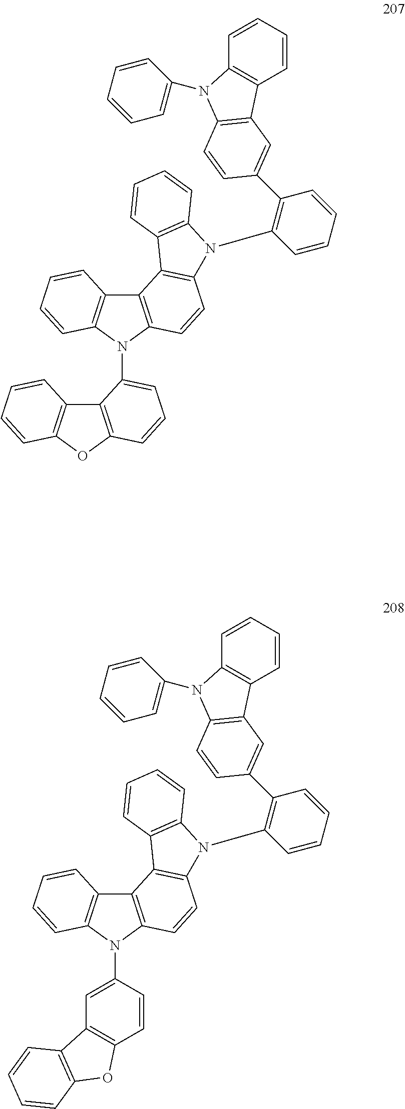 [SMILES: c1ccc(-n2c3ccccc3c3cc(-c4ccccc4-n4c5ccccc5c5c6c7ccccc7n(-c7cccc8oc9ccccc9c78)c6ccc54)ccc32)cc1 ; c1ccc(-n2c3ccccc3c3cc(-c4ccccc4-n4c5ccccc5c5c6c7ccccc7n(-c7ccc8oc9ccccc9c8c7)c6ccc54)ccc32)cc1]

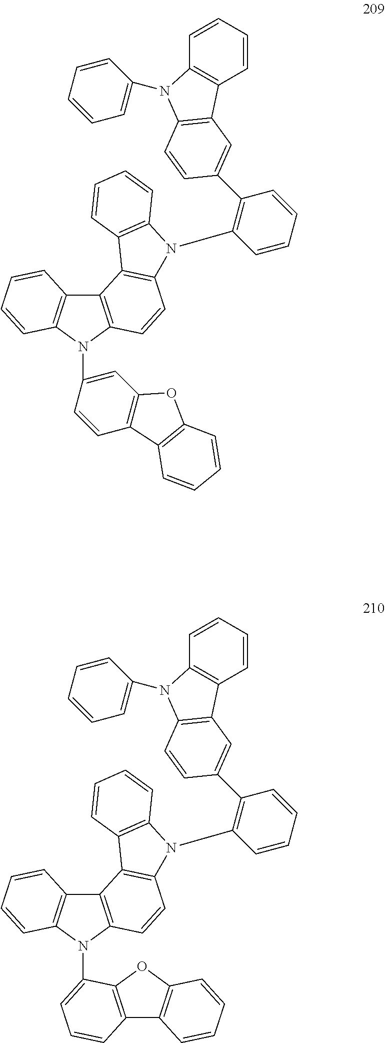 [SMILES: c1ccc(-n2c3ccccc3c3cc(-c4ccccc4-n4c5ccccc5c5c6c7ccccc7n(-c7ccc8c(c7)oc7ccccc78)c6ccc54)ccc32)cc1 ; c1ccc(-n2c3ccccc3c3cc(-c4ccccc4-n4c5ccccc5c5c6c7ccccc7n(-c7cccc8c7oc7ccccc78)c6ccc54)ccc32)cc1]

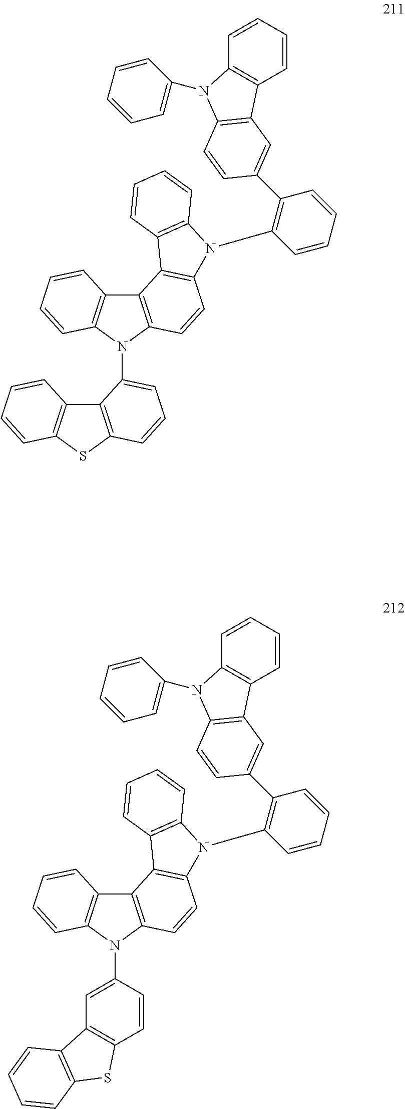 [SMILES: c1ccc(-n2c3ccccc3c3cc(-c4ccccc4-n4c5ccccc5c5c6c7ccccc7n(-c7cccc8sc9ccccc9c78)c6ccc54)ccc32)cc1 ; c1ccc(-n2c3ccccc3c3cc(-c4ccccc4-n4c5ccccc5c5c6c7ccccc7n(-c7ccc8sc9ccccc9c8c7)c6ccc54)ccc32)cc1]

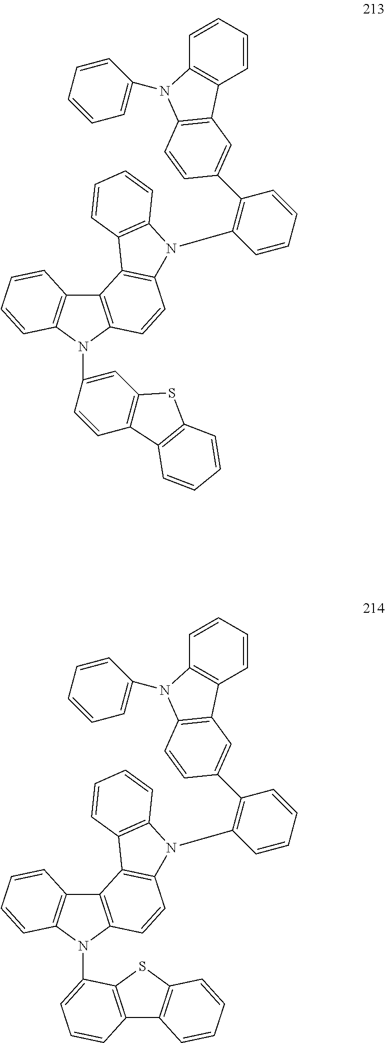 [SMILES: c1ccc(-n2c3ccccc3c3cc(-c4ccccc4-n4c5ccccc5c5c6c7ccccc7n(-c7ccc8c(c7)sc7ccccc78)c6ccc54)ccc32)cc1 ; c1ccc(-n2c3ccccc3c3cc(-c4ccccc4-n4c5ccccc5c5c6c7ccccc7n(-c7cccc8c7sc7ccccc78)c6ccc54)ccc32)cc1]

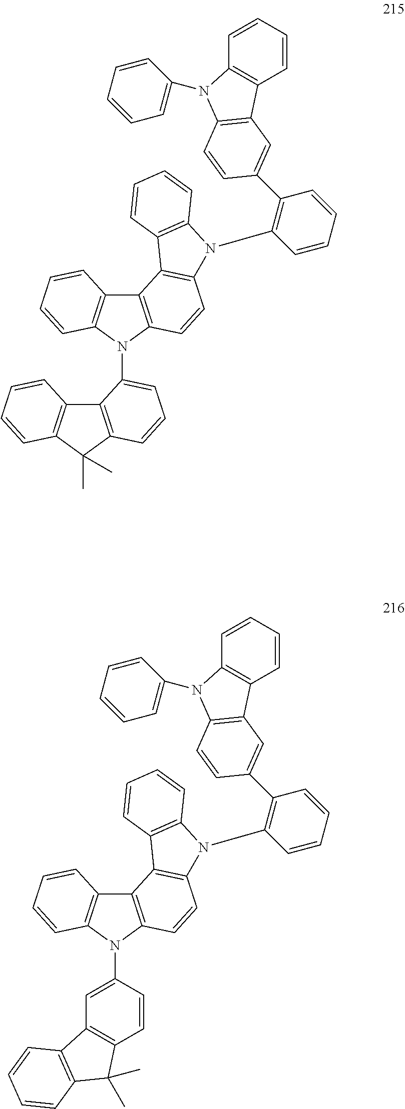 [SMILES: CC1(C)c2ccccc2-c2c(-n3c4ccccc4c4c5c6ccccc6n(-c6ccccc6-c6ccc7c(c6)c6ccccc6n7-c6ccccc6)c5ccc43)cccc21 ; CC1(C)c2ccccc2-c2cc(-n3c4ccccc4c4c5c6ccccc6n(-c6ccccc6-c6ccc7c(c6)c6ccccc6n7-c6ccccc6)c5ccc43)ccc21]

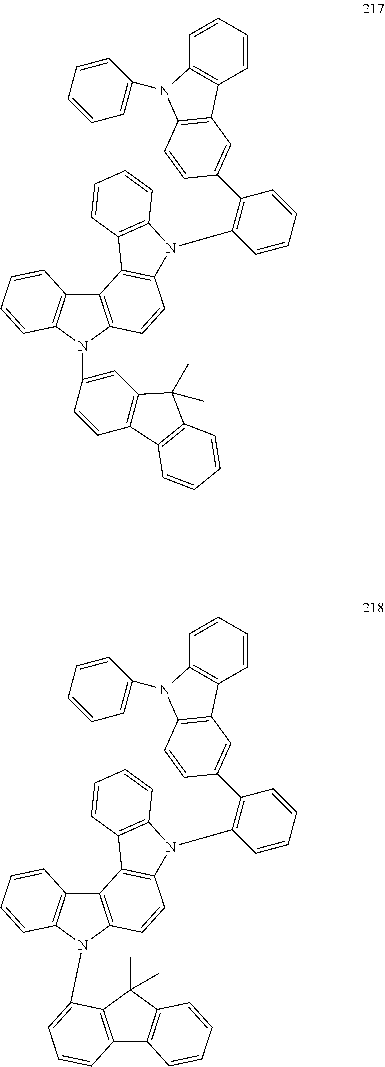 [SMILES: CC1(C)c2ccccc2-c2ccc(-n3c4ccccc4c4c5c6ccccc6n(-c6ccccc6-c6ccc7c(c6)c6ccccc6n7-c6ccccc6)c5ccc43)cc21 ; CC1(C)c2ccccc2-c2cccc(-n3c4ccccc4c4c5c6ccccc6n(-c6ccccc6-c6ccc7c(c6)c6ccccc6n7-c6ccccc6)c5ccc43)c21]

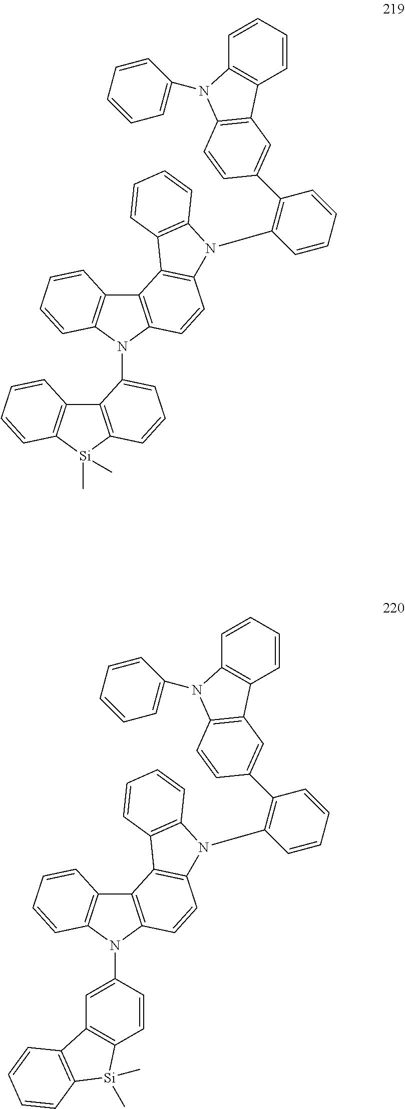 [SMILES: C[Si]1(C)c2ccccc2-c2c(-n3c4ccccc4c4c5c6ccccc6n(-c6ccccc6-c6ccc7c(c6)c6ccccc6n7-c6ccccc6)c5ccc43)cccc21 ; C[Si]1(C)c2ccccc2-c2cc(-n3c4ccccc4c4c5c6ccccc6n(-c6ccccc6-c6ccc7c(c6)c6ccccc6n7-c6ccccc6)c5ccc43)ccc21]

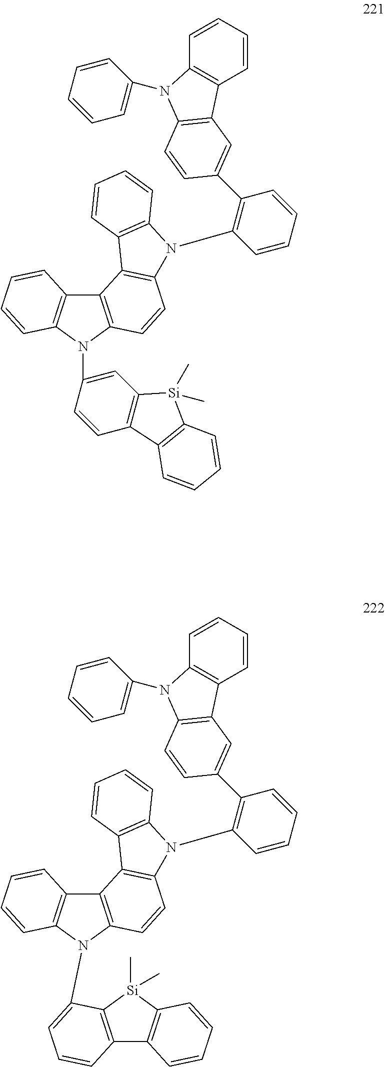 [SMILES: C[Si]1(C)c2ccccc2-c2ccc(-n3c4ccccc4c4c5c6ccccc6n(-c6ccccc6-c6ccc7c(c6)c6ccccc6n7-c6ccccc6)c5ccc43)cc21 ; C[Si]1(C)c2ccccc2-c2cccc(-n3c4ccccc4c4c5c6ccccc6n(-c6ccccc6-c6ccc7c(c6)c6ccccc6n7-c6ccccc6)c5ccc43)c21]

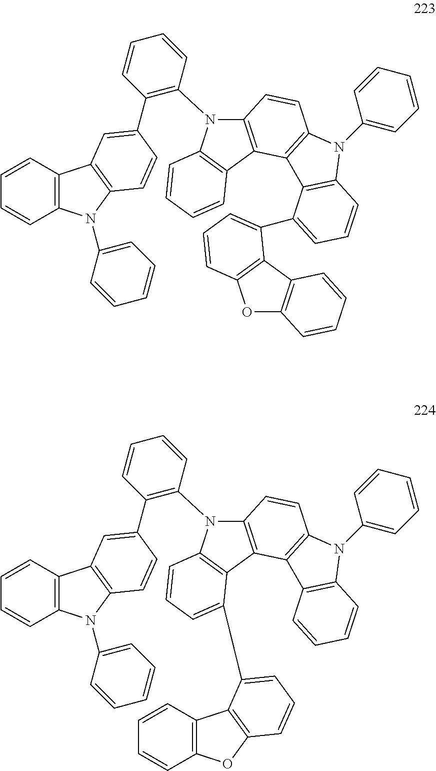 [SMILES: c1ccc(-n2c3ccccc3c3cc(-c4ccccc4-n4c5ccccc5c5c6c7c(-c8cccc9oc%10ccccc%10c89)cccc7n(-c7ccccc7)c6ccc54)ccc32)cc1 ; c1ccc(-n2c3ccccc3c3cc(-c4ccccc4-n4c5cccc(-c6cccc7oc8ccccc8c67)c5c5c6c7ccccc7n(-c7ccccc7)c6ccc54)ccc32)cc1]

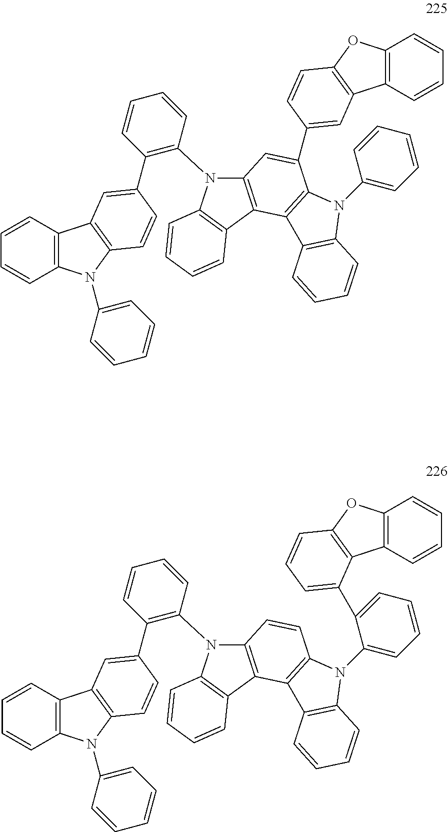 [SMILES: c1ccc(-n2c3ccccc3c3cc(-c4ccccc4-n4c5ccccc5c5c6c7ccccc7n(-c7ccccc7)c6c(-c6ccc7oc8ccccc8c7c6)cc54)ccc32)cc1 ; c1ccc(-n2c3ccccc3c3cc(-c4ccccc4-n4c5ccccc5c5c6c7ccccc7n(-c7ccccc7-c7cccc8oc9ccccc9c78)c6ccc54)ccc32)cc1]

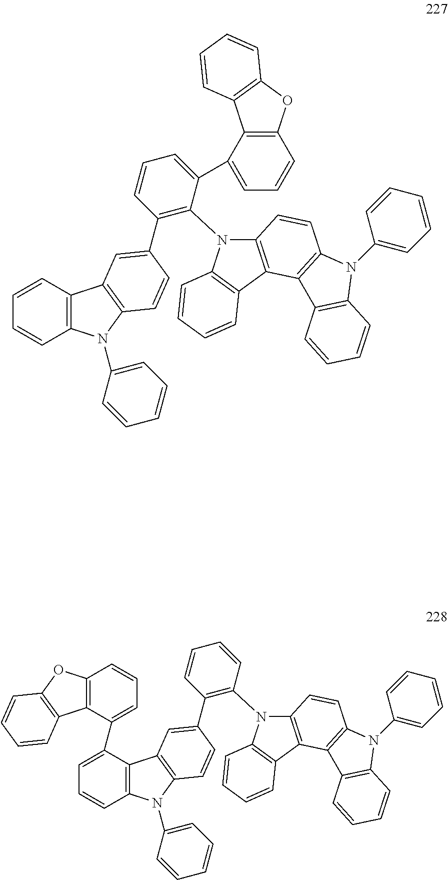 [SMILES: c1ccc(-n2c3ccccc3c3cc(-c4cccc(-c5cccc6oc7ccccc7c56)c4-n4c5ccccc5c5c6c7ccccc7n(-c7ccccc7)c6ccc54)ccc32)cc1 ; c1ccc(-n2c3ccc(-c4ccccc4-n4c5ccccc5c5c6c7ccccc7n(-c7ccccc7)c6ccc54)cc3c3c(-c4cccc5oc6ccccc6c45)cccc32)cc1]

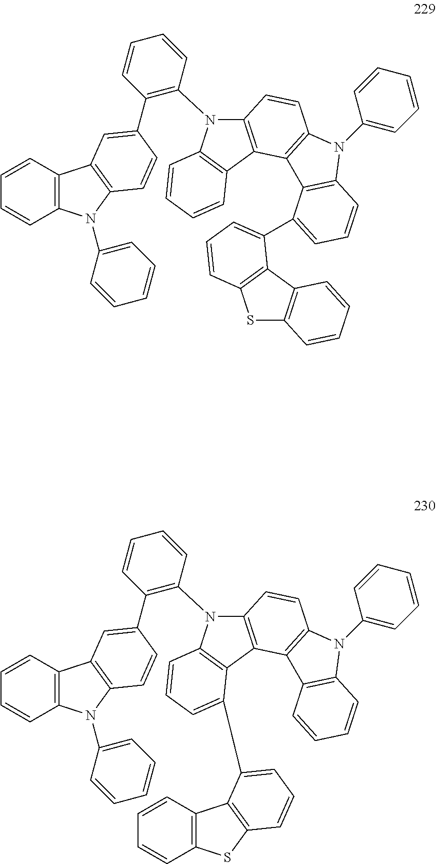 [SMILES: c1ccc(-n2c3ccccc3c3cc(-c4ccccc4-n4c5ccccc5c5c6c7c(-c8cccc9sc%10ccccc%10c89)cccc7n(-c7ccccc7)c6ccc54)ccc32)cc1 ; c1ccc(-n2c3ccccc3c3cc(-c4ccccc4-n4c5cccc(-c6cccc7sc8ccccc8c67)c5c5c6c7ccccc7n(-c7ccccc7)c6ccc54)ccc32)cc1]

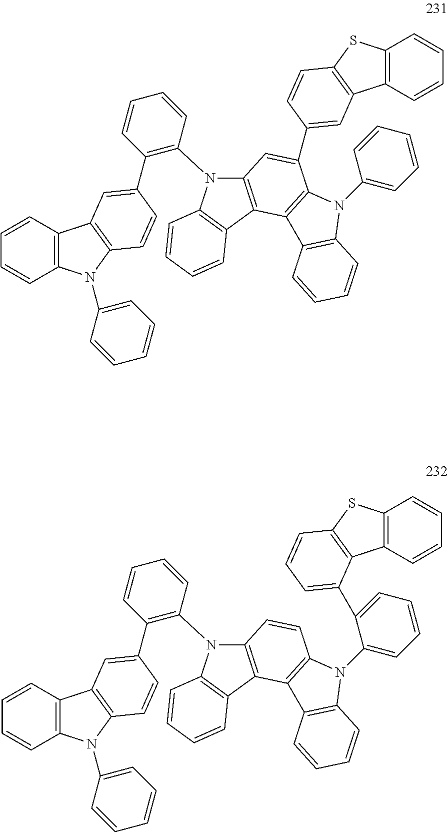 [SMILES: c1ccc(-n2c3ccccc3c3cc(-c4ccccc4-n4c5ccccc5c5c6c7ccccc7n(-c7ccccc7)c6c(-c6ccc7sc8ccccc8c7c6)cc54)ccc32)cc1 ; c1ccc(-n2c3ccccc3c3cc(-c4ccccc4-n4c5ccccc5c5c6c7ccccc7n(-c7ccccc7-c7cccc8sc9ccccc9c78)c6ccc54)ccc32)cc1]

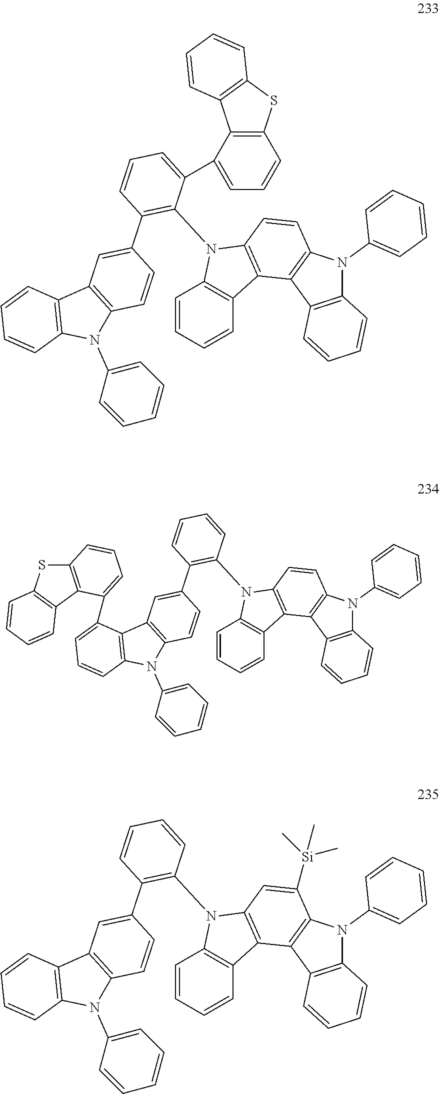 [SMILES: c1ccc(-n2c3ccccc3c3cc(-c4cccc(-c5cccc6sc7ccccc7c56)c4-n4c5ccccc5c5c6c7ccccc7n(-c7ccccc7)c6ccc54)ccc32)cc1 ; c1ccc(-n2c3ccc(-c4ccccc4-n4c5ccccc5c5c6c7ccccc7n(-c7ccccc7)c6ccc54)cc3c3c(-c4cccc5sc6ccccc6c45)cccc32)cc1 ; C[Si](C)(C)c1cc2c(c3ccccc3n2-c2ccccc2-c2ccc3c(c2)c2ccccc2n3-c2ccccc2)c2c3ccccc3n(-c3ccccc3)c12]

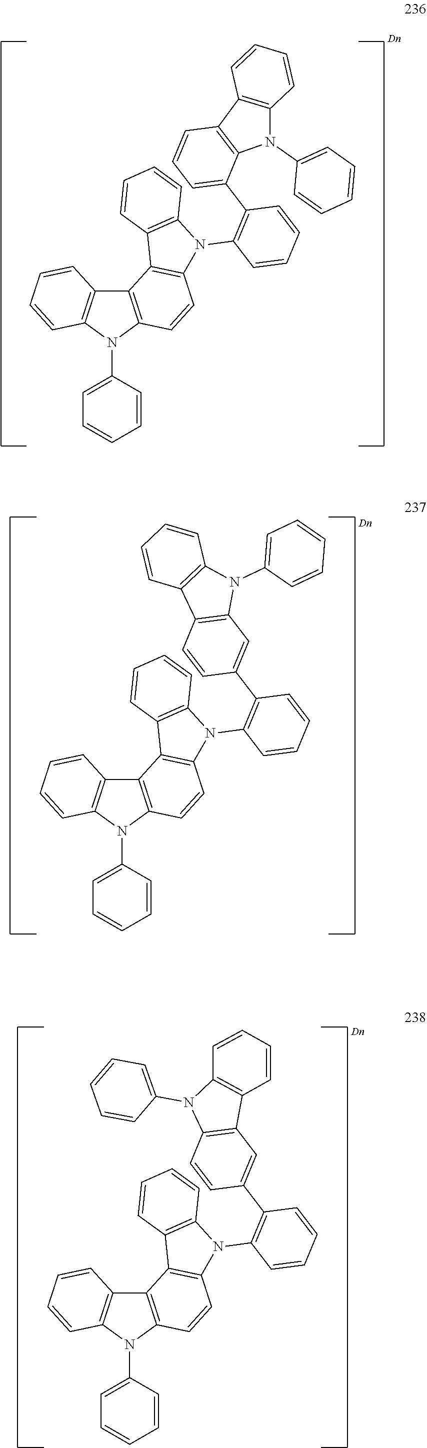

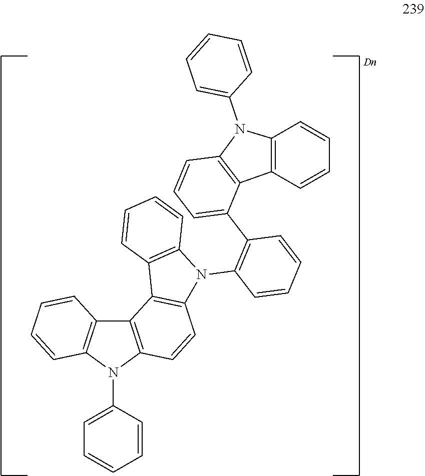

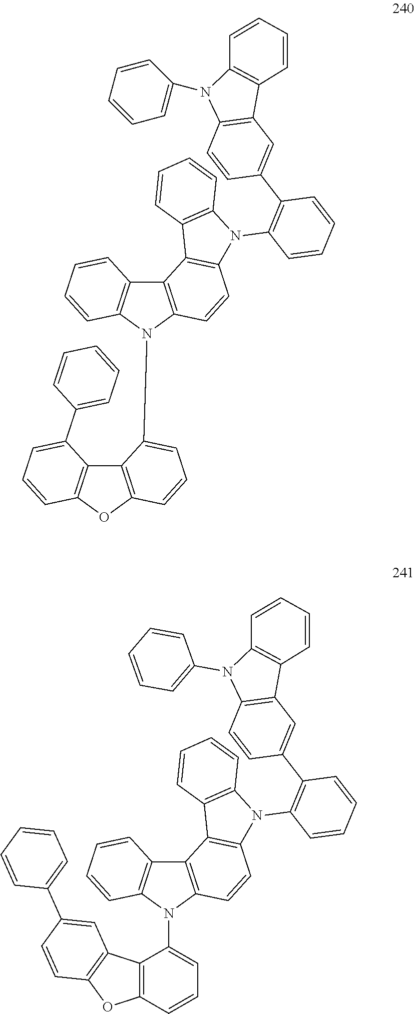 [SMILES: c1ccc(-c2cccc3oc4cccc(-n5c6ccccc6c6c7c8ccccc8n(-c8ccccc8-c8ccc9c(c8)c8ccccc8n9-c8ccccc8)c7ccc65)c4c23)cc1 ; c1ccc(-c2ccc3oc4cccc(-n5c6ccccc6c6c7c8ccccc8n(-c8ccccc8-c8ccc9c(c8)c8ccccc8n9-c8ccccc8)c7ccc65)c4c3c2)cc1]

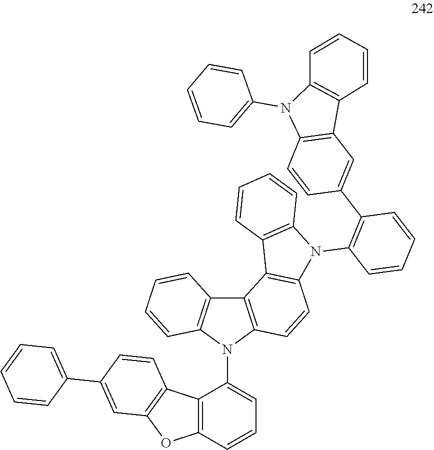 [SMILES: c1ccc(-c2ccc3c(c2)oc2cccc(-n4c5ccccc5c5c6c7ccccc7n(-c7ccccc7-c7ccc8c(c7)c7ccccc7n8-c7ccccc7)c6ccc54)c23)cc1]

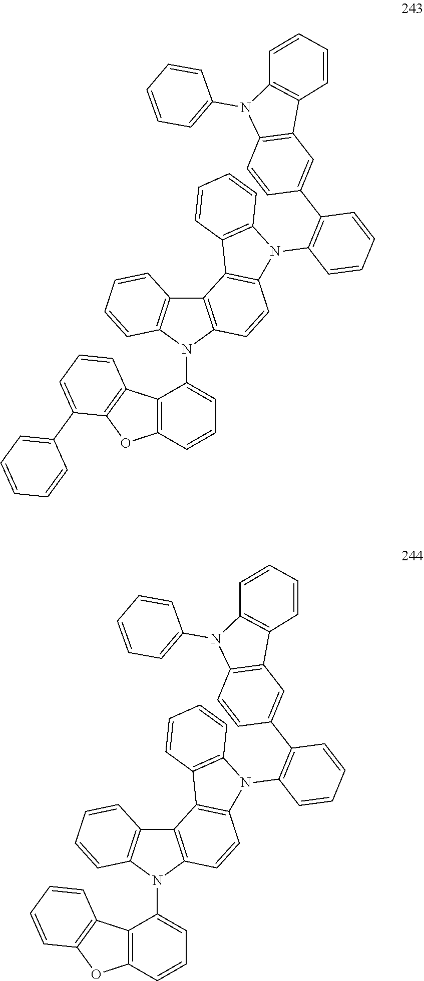 [SMILES: c1ccc(-c2cccc3c2oc2cccc(-n4c5ccccc5c5c6c7ccccc7n(-c7ccccc7-c7ccc8c(c7)c7ccccc7n8-c7ccccc7)c6ccc54)c23)cc1 ; c1ccc(-n2c3ccccc3c3cc(-c4ccccc4-n4c5ccccc5c5c6c7ccccc7n(-c7cccc8oc9ccccc9c78)c6ccc54)ccc32)cc1]

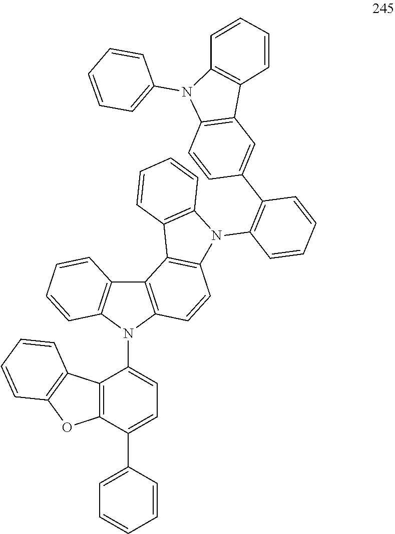 [SMILES: c1ccc(-c2ccc(-n3c4ccccc4c4c5c6ccccc6n(-c6ccccc6-c6ccc7c(c6)c6ccccc6n7-c6ccccc6)c5ccc43)c3c2oc2ccccc23)cc1]

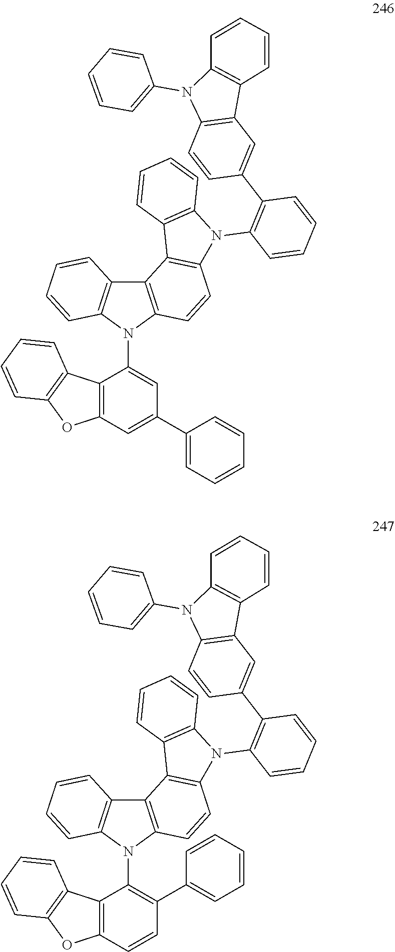 [SMILES: c1ccc(-c2cc(-n3c4ccccc4c4c5c6ccccc6n(-c6ccccc6-c6ccc7c(c6)c6ccccc6n7-c6ccccc6)c5ccc43)c3c(c2)oc2ccccc23)cc1 ; c1ccc(-c2ccc3oc4ccccc4c3c2-n2c3ccccc3c3c4c5ccccc5n(-c5ccccc5-c5ccc6c(c5)c5ccccc5n6-c5ccccc5)c4ccc32)cc1]

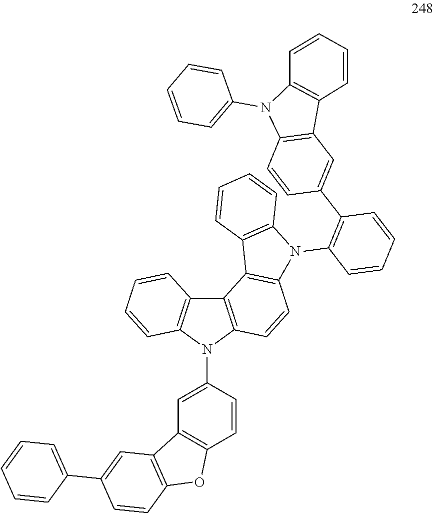 [SMILES: c1ccc(-c2ccc3oc4ccc(-n5c6ccccc6c6c7c8ccccc8n(-c8ccccc8-c8ccc9c(c8)c8ccccc8n9-c8ccccc8)c7ccc65)cc4c3c2)cc1]

(Dn refers to the number of deuterium substitutions and indicates a structure substituted with one or more deuterium atoms). However, as noted above, any hydrogen in any compound may be protium, deuterium, or tritium, based on natural or artificial substitution.

A composition for an organic optoelectronic device according to some example embodiments may include a first compound and a second compound. The first compound may be the aforementioned compound for an organic optoelectronic device. In an implementation, the second compound may be represented by, e.g., Chemical Formula 2.

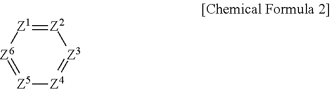

In Chemical Formula 2, Z¹ to Z⁶ may each independently be, e.g., N or C-L^{a}-R^{a}. In an implementation, at least two of Z¹ to Z⁶ are N.

Each L^{a} may independently be or include, e.g., a single bond, a substituted or unsubstituted C6 to C20 arylene group, a substituted or unsubstituted C2 to C20 heterocyclic group, or a combination thereof.

Each R^{a} may independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

Each R^{a} may be separately present or adjacent groups thereof may be linked to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring.

The second compound may help effectively expand the LUMO energy band by including a nitrogen-containing hexagonal or six-membered ring moiety, and thus it may be included together with the aforementioned first compound to increase the balance of holes and electrons, thereby significantly improving the life-span characteristics of the device to which it is applied.

In an implementation, two of Z¹ to Z⁶ may be nitrogen (N) and the remainders may be C-L^{a}-R^{a}.

In an implementation, Z¹ and Z³ may be nitrogen, Z² may be N or C-L^{a}-R^{a}, Z⁴ may be N or C-L^{a}-R^{a}, Z⁵ may be N or C-L^{a}-R^{a}, and Z⁶ may be N or C-L^{a}-R^{a}.

In an implementation, three of Z¹ to Z⁶ may be nitrogen (N) and the remainders may be C-L^{a}-R^{a}.

In an implementation, Z¹, Z³, and Z⁵ may be nitrogen, Z² may be N or C-L^{a}-R^{a}, Z⁴ may be N or C-L^{a}-R^{a}, and Z⁶ may be N or C-L^{a}-R^{a}.

In an implementation, depending on the substituent of R^{a}, the second compound may be represented by, e.g., one of Chemical Formula 2A to Chemical Formula 2C.

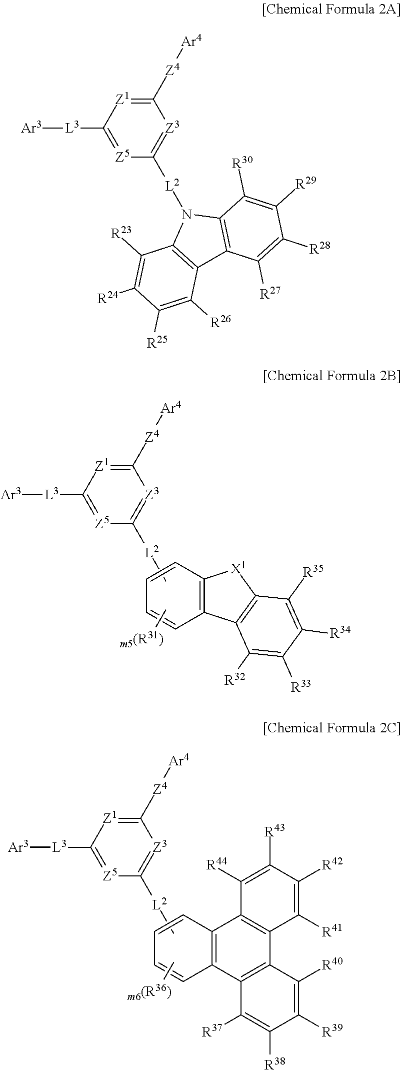

In Chemical Formulas 2A to 2C, Z¹, Z³, and Z⁵ may each independently be, e.g., N or C-L^{a}-R^{a}. In an implementation, at least two of Z¹, Z³, and Z⁵ are N.

X¹ may be, e.g., O, S or NR^{b}.

L^{a}, and L² to L⁴ may each independently be, e.g., a single bond, a substituted or unsubstituted C6 to C20 arylene group, a substituted or unsubstituted C2 to C20 heterocyclic group, or a combination thereof.

R^{a}, R^{b}, and R²³ to R⁴⁴ may each independently be, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

R²³ to R³⁰ may be separately present or adjacent groups thereof may be linked to form a substituted or unsubstituted aromatic monocyclic or polycyclic ring.

R³¹ to R³⁵ may be separately present or adjacent groups thereof are linked to form a substituted or unsubstituted aromatic monocyclic or polycyclic ring.

Ar³ and Ar⁴ may each independently be, e.g., a substituted or unsubstituted C6 to C30 aryl group, a substituted or unsubstituted C2 to C30 heterocyclic group, or a combination thereof.

R^{a}, Ar³, and Ar⁴ may be separately present, or adjacent groups of R^{a}, Ar³, and Ar⁴ may be linked to form a substituted or unsubstituted aromatic or heteroaromatic monocyclic or polycyclic ring.

m5 and m6 may each independently be, e.g., an integer of 1 to 3.

In Chemical Formula 2B, m5 may be 2 or more, and each R³¹ may be the same or different.

In Chemical Formula 2C, m6 may be 2 or more, and each R³⁶ may be the same or different.

In the present specification, “adjacent groups may be linked to form a substituted or unsubstituted aromatic or heteroaromatic monocyclic or polycyclic ring” means that any two adjacent substituents are linked to each other to form a ring. In an implementation, in Chemical Formula 2A, adjacent groups among R²³ to R³⁰ may be linked to each other to form a substituted or unsubstituted aromatic monocyclic ring. The aromatic monocyclic ring formed may include, e.g., a substituted or unsubstituted phenyl group.

In an implementation, Chemical Formula 2A may be represented by, e.g., one of Chemical Formula 2A-I to Chemical Formula 2A-XVIII.

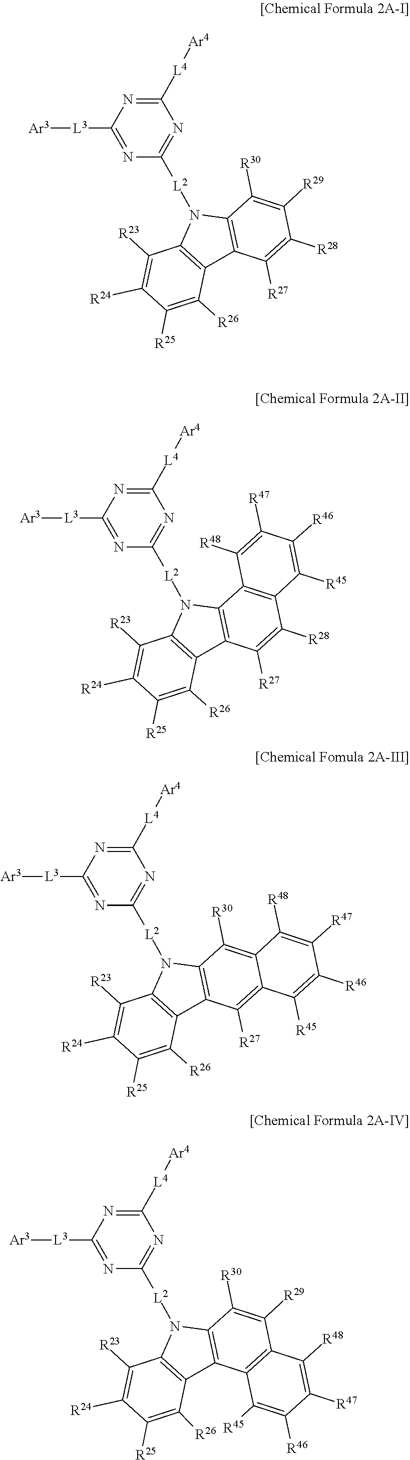

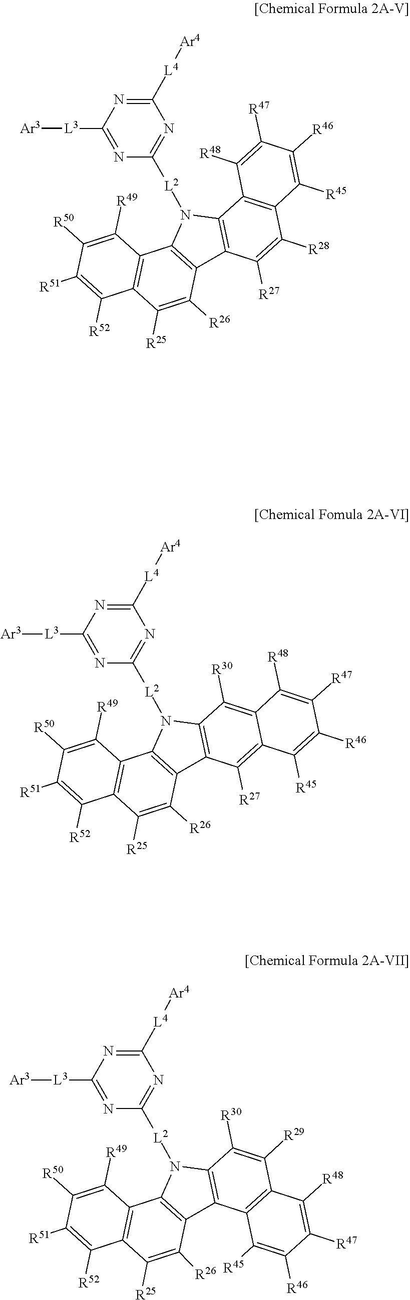

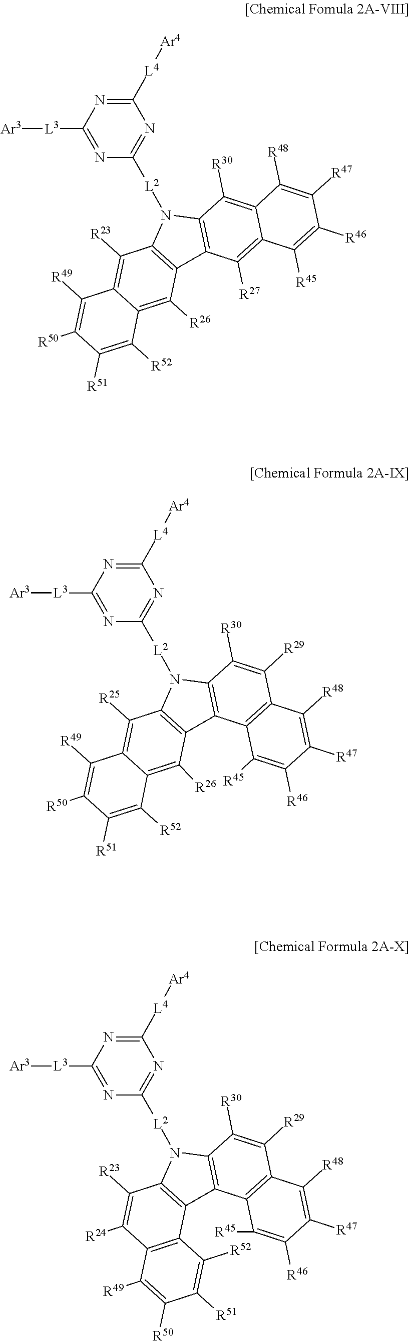

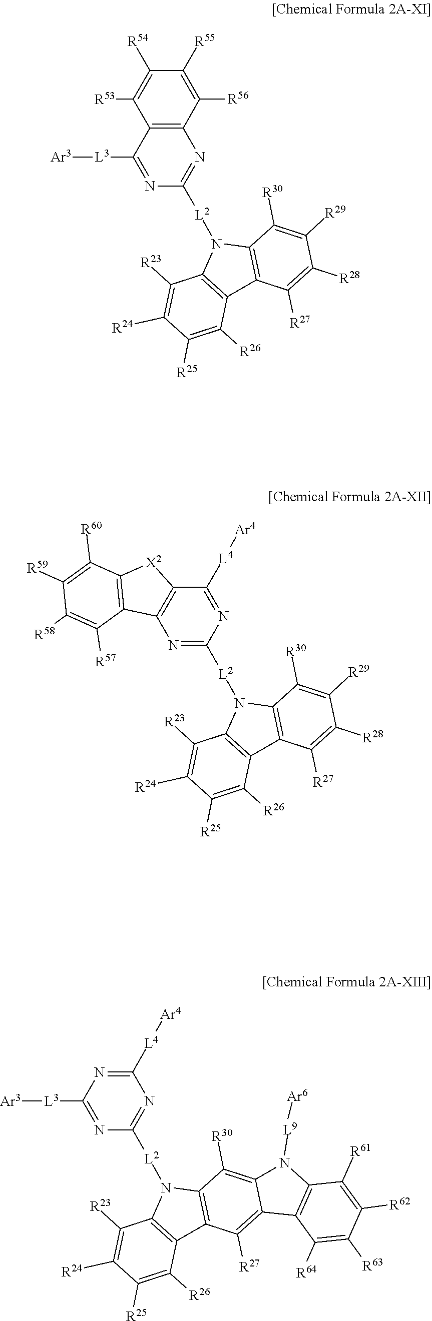

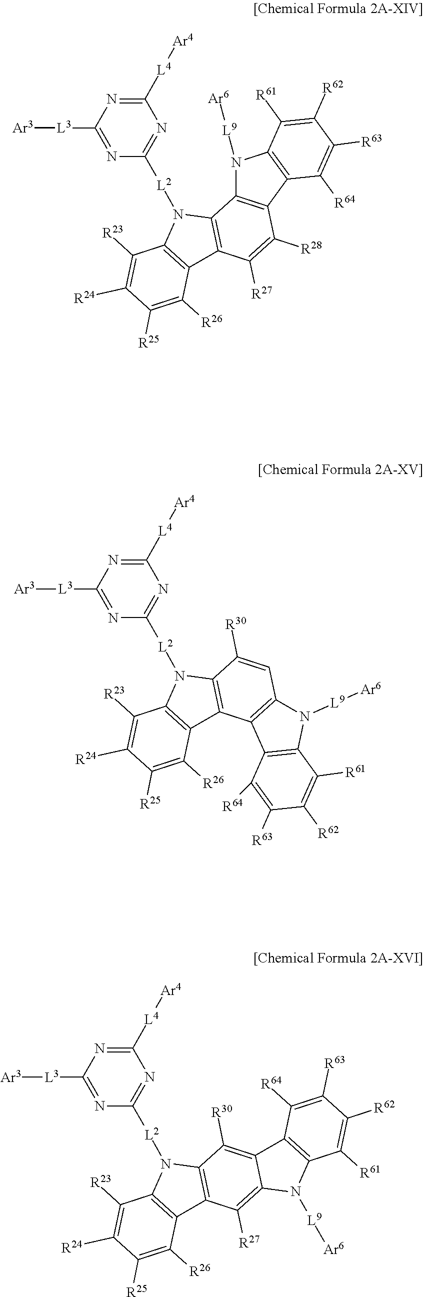

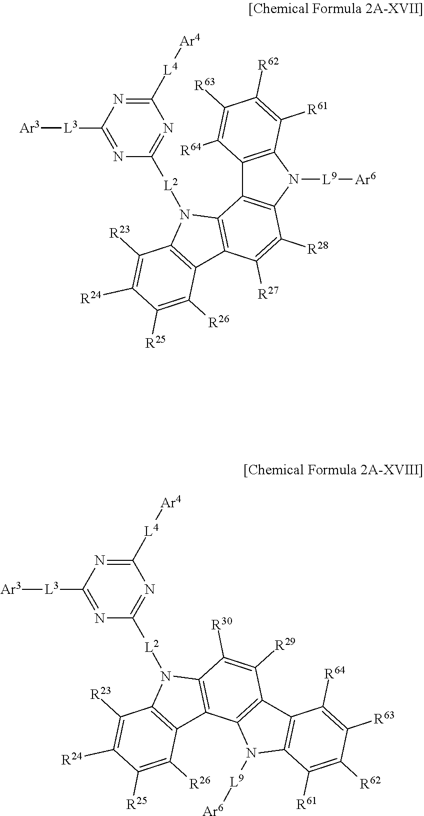

In Chemical Formula 2A-I to Chemical Formula 2A-XVIII, L² to L⁴, Ar³ and Ar⁴, R²³ to R³⁰ may be the same as described above.

Ar⁶ may be, e.g., a substituted or unsubstituted C6 to C30 aryl group, or a substituted or unsubstituted C2 to C30 heterocyclic group.

L⁹ may be, e.g., a single bond, a substituted or unsubstituted C6 to C20 arylene group, or a substituted or unsubstituted C2 to C20 heterocyclic group.

R⁴⁵ to R⁶⁴ may each independently be, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted C1 to C10 alkylsilyl group, a substituted or unsubstituted amine group, a halogen, a cyano group, or a combination thereof.

In an implementation, L² to L⁴ may each independently be, e.g., a single bond, a substituted or unsubstituted phenylene group, a substituted or unsubstituted biphenylene group, a substituted or unsubstituted naphthylene group, a substituted or unsubstituted dibenzofuranylene group, or a substituted or unsubstituted dibenzothiophenylene group.

In an implementation, Ar³, Ar⁴ and Ar⁶ may each independently be, e.g., a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted triphenylene group, a substituted or unsubstituted carbazolyl group, a substituted or unsubstituted dibenzofuranyl group, or a substituted or unsubstituted dibenzothiophenyl group.

In an implementation, R²³ to R³⁰ and R⁴⁵ to R⁶⁴ may each independently be, e.g., hydrogen, deuterium, cyano group, a substituted or unsubstituted C1 to C10 alkyl group, a substituted or unsubstituted C1 to C10 alkylsilyl group, a substituted or unsubstituted phenyl group, a substituted or unsubstituted dibenzofuranyl group, or a substituted or unsubstituted dibenzothiophenyl group.

In an implementation, Chemical Formula 2B may be represented by, e.g., one of Chemical Formula 2B-I to Chemical Formula 2B-IV.

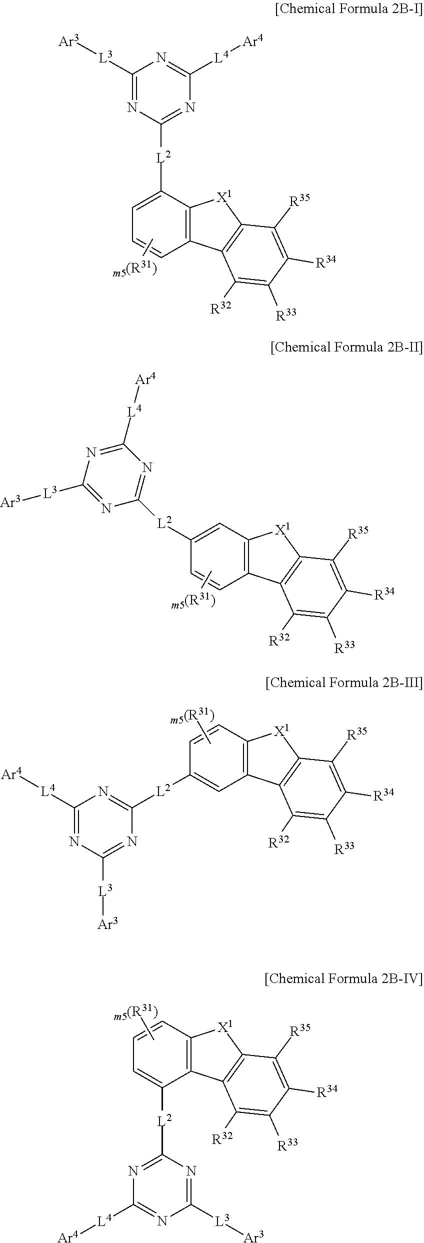

In Chemical Formula 2B-I to Chemical Formula 2B-IV, L² to L⁴, Ar³ and Ar⁴, R³¹ to R³⁵, and m5 may be defined the same as described above.

In an implementation, Chemical Formula 2C may be represented by Chemical Formula 2C-I or Chemical Formula 2C-II.

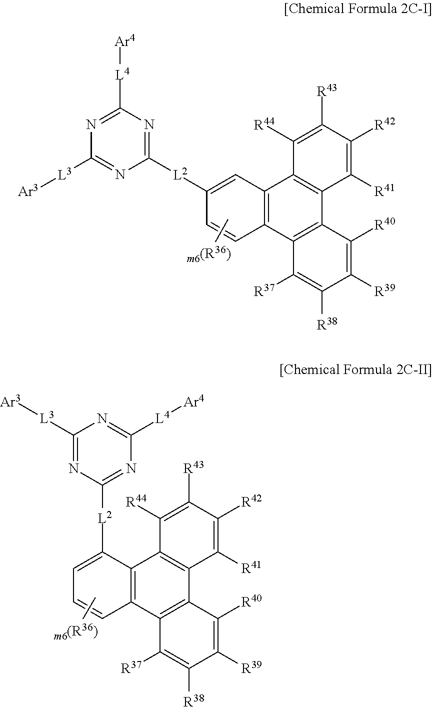

In Chemical Formula 2C-I and Chemical Formula 2C-II, L² to L⁴, Ar³ and Ar⁴, R³⁶ to R⁴⁴, and m6 may be defined the same as described above.

In an implementation, Chemical Formula 2 may be represented by, e.g., one of Chemical Formula 2A-XIV or Chemical Formula 2C-I.

In an implementation, in Chemical Formula 2A-XIV, L² to L⁴ and L⁹ may each independently be, e.g., a single bond or a substituted or unsubstituted C6 to C12 aryl group, Ar³, Ar⁴, and Ar⁶ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group or a substituted or unsubstituted carbazolyl group, and R²³ to R²⁸, and R⁶¹ to R⁶⁴ may each independently be, e.g., hydrogen, deuterium, a substituted or unsubstituted C1 to C10 alkyl group, or a substituted or unsubstituted C6 to C12 aryl group.

In an implementation, in Chemical Formula 2C-I, L² to L⁴ may each independently be a single bond or a substituted or unsubstituted C6 to C12 aryl group, Ar³ and Ar⁴ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, and R³⁶ to R⁴⁴ may each independently be, e.g., hydrogen, deuterium, a substituted or unsubstituted C1 to C10 alkyl group, or a substituted or unsubstituted C6 to C12 aryl group.

In an implementation, the second compound may be, e.g., a compound of Group 2.

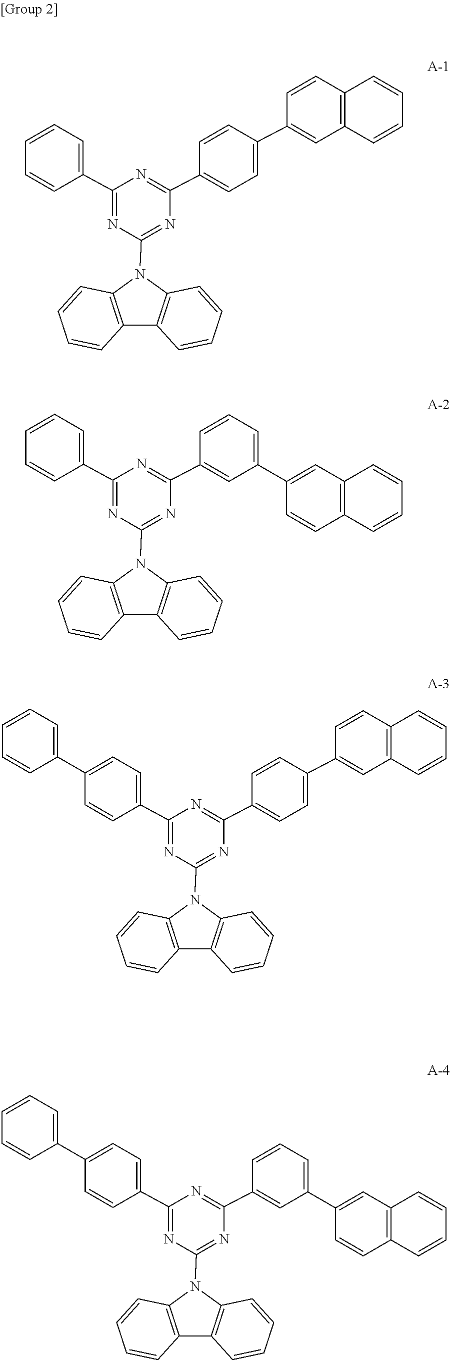 [SMILES: c1ccc(-c2nc(-c3ccc(-c4ccc5ccccc5c4)cc3)nc(-n3c4ccccc4c4ccccc43)n2)cc1 ; c1ccc(-c2nc(-c3cccc(-c4ccc5ccccc5c4)c3)nc(-n3c4ccccc4c4ccccc43)n2)cc1 ; c1ccc(-c2ccc(-c3nc(-c4ccc(-c5ccc6ccccc6c5)cc4)nc(-n4c5ccccc5c5ccccc54)n3)cc2)cc1 ; c1ccc(-c2ccc(-c3nc(-c4cccc(-c5ccc6ccccc6c5)c4)nc(-n4c5ccccc5c5ccccc54)n3)cc2)cc1]

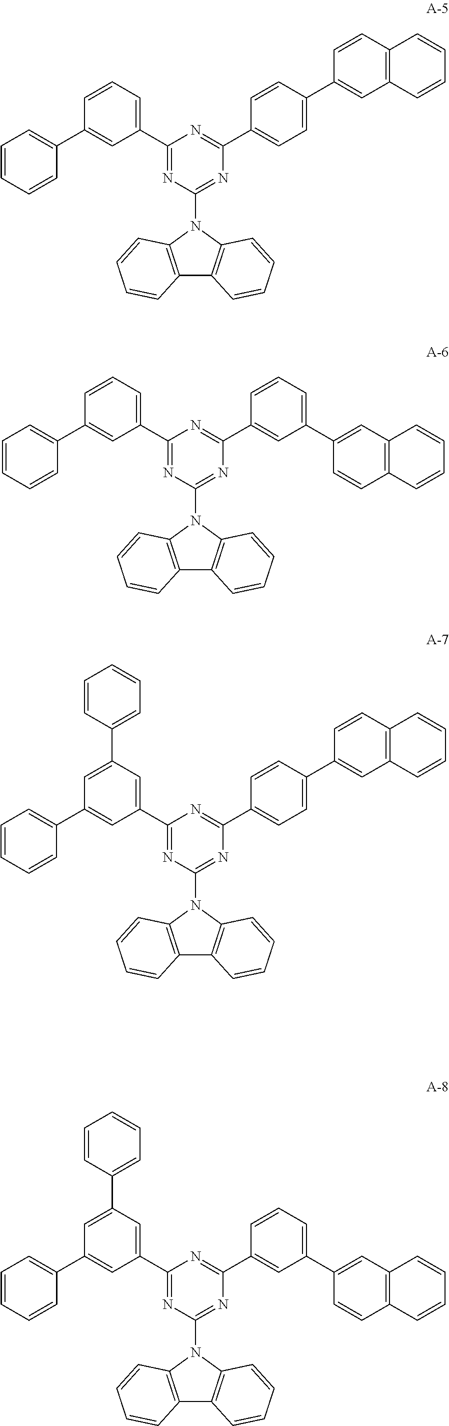 [SMILES: c1ccc(-c2cccc(-c3nc(-c4ccc(-c5ccc6ccccc6c5)cc4)nc(-n4c5ccccc5c5ccccc54)n3)c2)cc1 ; c1ccc(-c2cccc(-c3nc(-c4cccc(-c5ccc6ccccc6c5)c4)nc(-n4c5ccccc5c5ccccc54)n3)c2)cc1 ; c1ccc(-c2cc(-c3ccccc3)cc(-c3nc(-c4ccc(-c5ccc6ccccc6c5)cc4)nc(-n4c5ccccc5c5ccccc54)n3)c2)cc1 ; c1ccc(-c2cc(-c3ccccc3)cc(-c3nc(-c4cccc(-c5ccc6ccccc6c5)c4)nc(-n4c5ccccc5c5ccccc54)n3)c2)cc1]

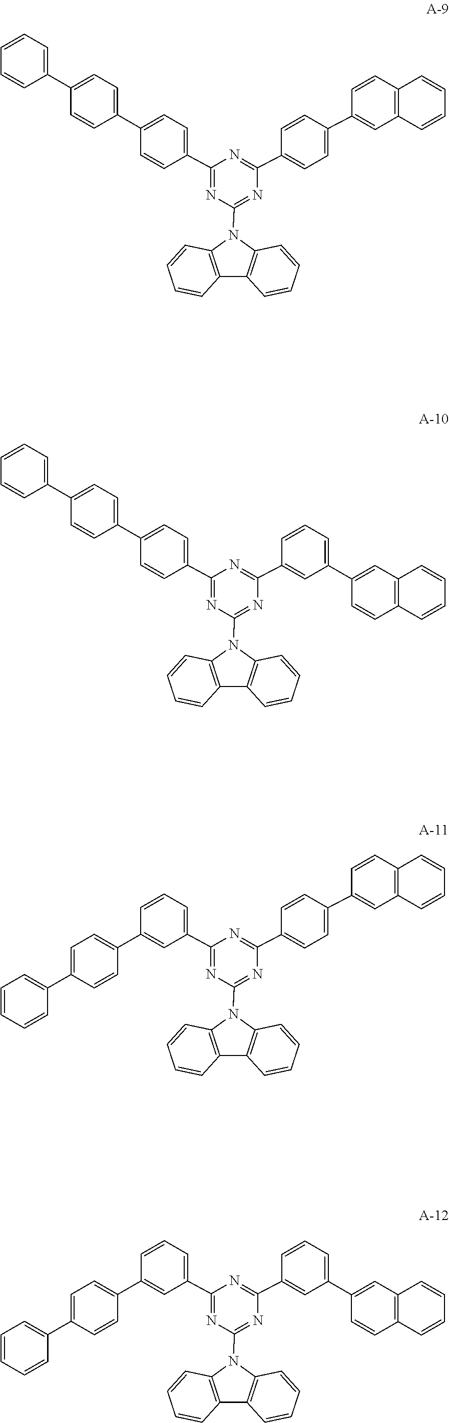 [SMILES: c1ccc(-c2ccc(-c3ccc(-c4nc(-c5ccc(-c6ccc7ccccc7c6)cc5)nc(-n5c6ccccc6c6ccccc65)n4)cc3)cc2)cc1 ; c1ccc(-c2ccc(-c3ccc(-c4nc(-c5cccc(-c6ccc7ccccc7c6)c5)nc(-n5c6ccccc6c6ccccc65)n4)cc3)cc2)cc1 ; c1ccc(-c2ccc(-c3cccc(-c4nc(-c5ccc(-c6ccc7ccccc7c6)cc5)nc(-n5c6ccccc6c6ccccc65)n4)c3)cc2)cc1 ; c1ccc(-c2ccc(-c3cccc(-c4nc(-c5cccc(-c6ccc7ccccc7c6)c5)nc(-n5c6ccccc6c6ccccc65)n4)c3)cc2)cc1]

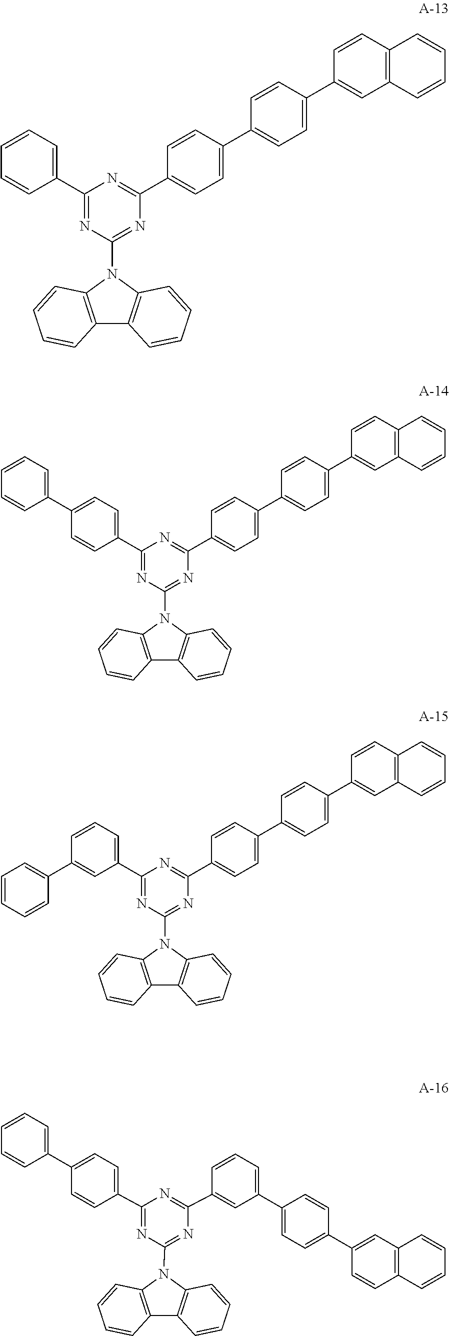 [SMILES: c1ccc(-c2nc(-c3ccc(-c4ccc(-c5ccc6ccccc6c5)cc4)cc3)nc(-n3c4ccccc4c4ccccc43)n2)cc1 ; c1ccc(-c2ccc(-c3nc(-c4ccc(-c5ccc(-c6ccc7ccccc7c6)cc5)cc4)nc(-n4c5ccccc5c5ccccc54)n3)cc2)cc1 ; c1ccc(-c2cccc(-c3nc(-c4ccc(-c5ccc(-c6ccc7ccccc7c6)cc5)cc4)nc(-n4c5ccccc5c5ccccc54)n3)c2)cc1 ; c1ccc(-c2ccc(-c3nc(-c4cccc(-c5ccc(-c6ccc7ccccc7c6)cc5)c4)nc(-n4c5ccccc5c5ccccc54)n3)cc2)cc1]

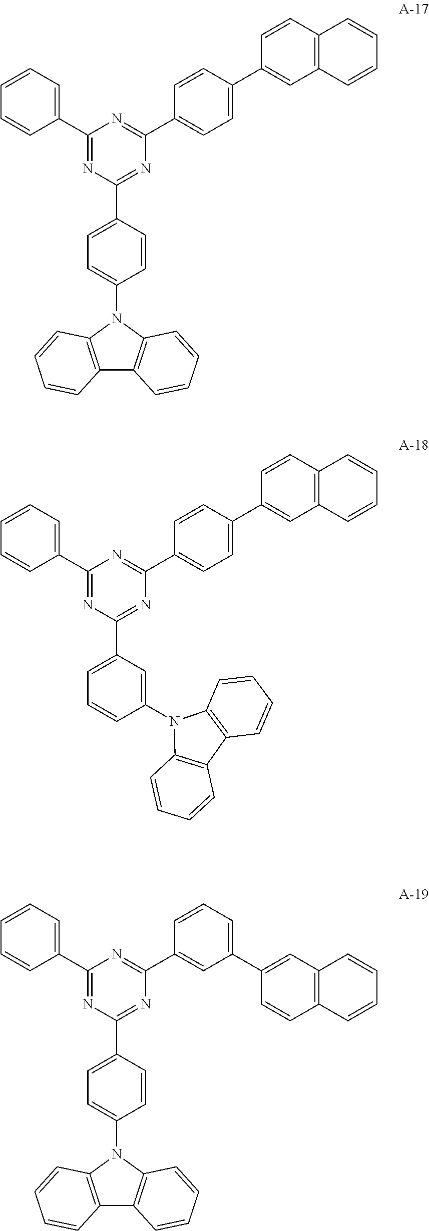 [SMILES: c1ccc(-c2nc(-c3ccc(-c4ccc5ccccc5c4)cc3)nc(-c3ccc(-n4c5ccccc5c5ccccc54)cc3)n2)cc1 ; c1ccc(-c2nc(-c3ccc(-c4ccc5ccccc5c4)cc3)nc(-c3cccc(-n4c5ccccc5c5ccccc54)c3)n2)cc1 ; c1ccc(-c2nc(-c3ccc(-n4c5ccccc5c5ccccc54)cc3)nc(-c3cccc(-c4ccc5ccccc5c4)c3)n2)cc1]

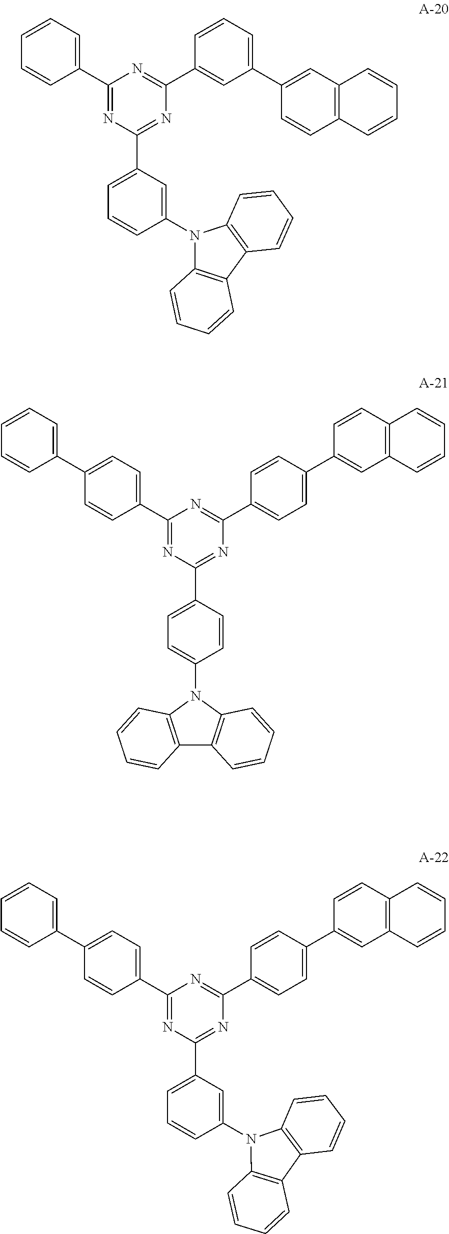 [SMILES: c1ccc(-c2nc(-c3cccc(-c4ccc5ccccc5c4)c3)nc(-c3cccc(-n4c5ccccc5c5ccccc54)c3)n2)cc1 ; c1ccc(-c2ccc(-c3nc(-c4ccc(-c5ccc6ccccc6c5)cc4)nc(-c4ccc(-n5c6ccccc6c6ccccc65)cc4)n3)cc2)cc1 ; c1ccc(-c2ccc(-c3nc(-c4ccc(-c5ccc6ccccc6c5)cc4)nc(-c4cccc(-n5c6ccccc6c6ccccc65)c4)n3)cc2)cc1]

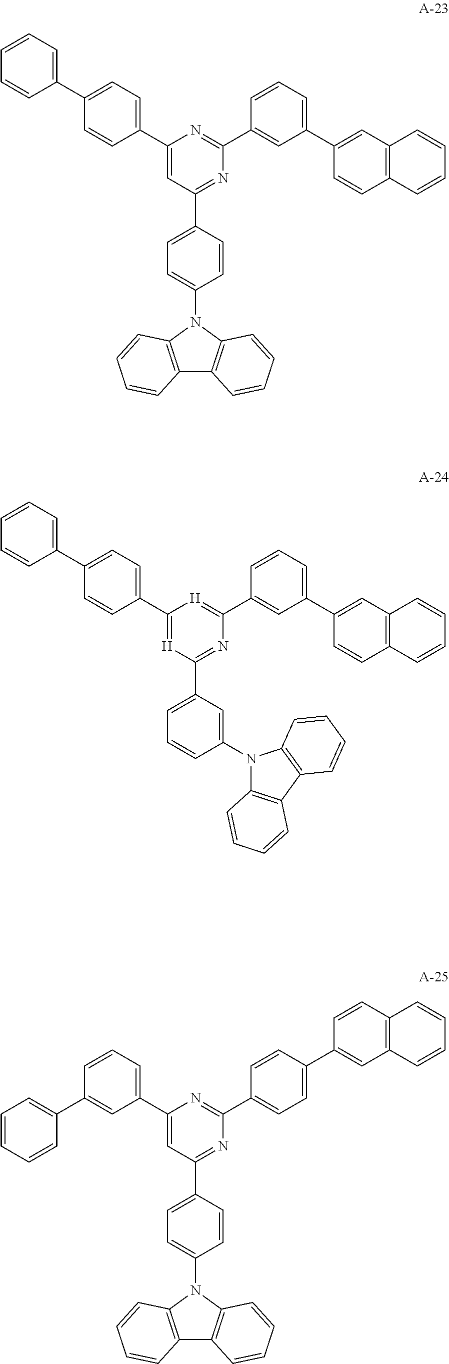

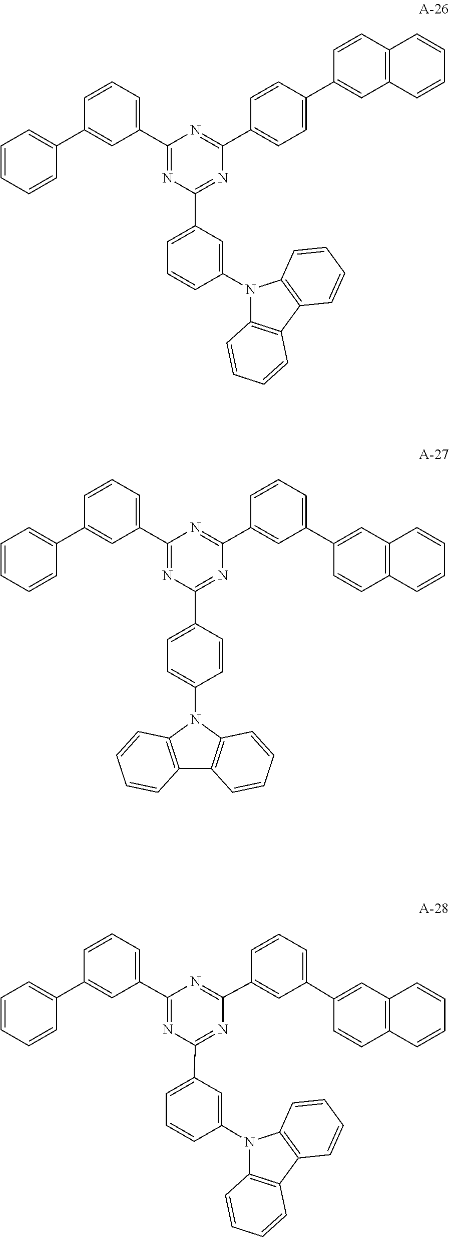 [SMILES: c1ccc(-c2cccc(-c3nc(-c4ccc(-c5ccc6ccccc6c5)cc4)nc(-c4cccc(-n5c6ccccc6c6ccccc65)c4)n3)c2)cc1 ; c1ccc(-c2cccc(-c3nc(-c4ccc(-n5c6ccccc6c6ccccc65)cc4)nc(-c4cccc(-c5ccc6ccccc6c5)c4)n3)c2)cc1 ; c1ccc(-c2cccc(-c3nc(-c4cccc(-c5ccc6ccccc6c5)c4)nc(-c4cccc(-n5c6ccccc6c6ccccc65)c4)n3)c2)cc1]

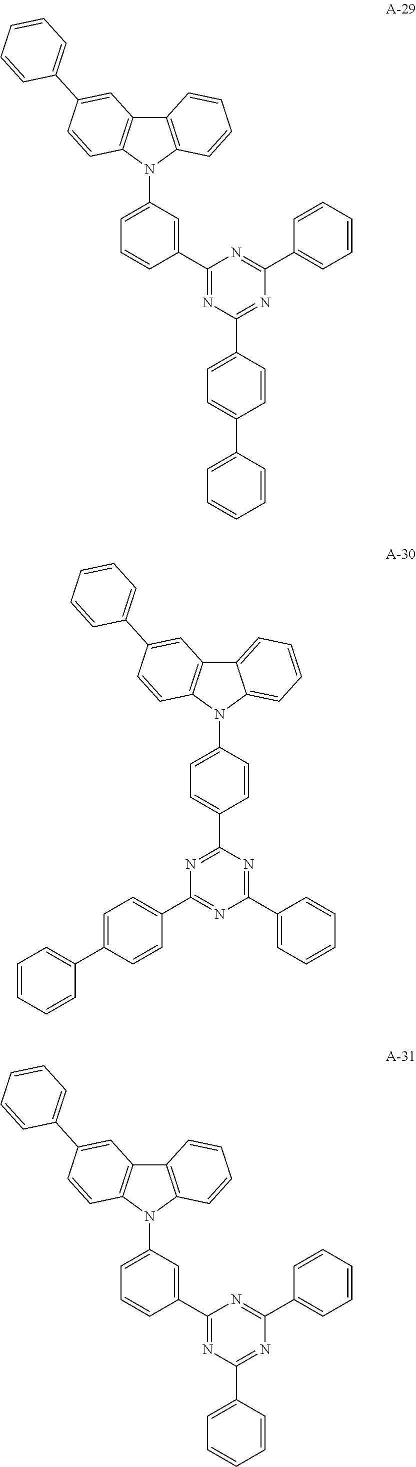 [SMILES: c1ccc(-c2ccc(-c3nc(-c4ccccc4)nc(-c4cccc(-n5c6ccccc6c6cc(-c7ccccc7)ccc65)c4)n3)cc2)cc1 ; c1ccc(-c2ccc(-c3nc(-c4ccccc4)nc(-c4ccc(-n5c6ccccc6c6cc(-c7ccccc7)ccc65)cc4)n3)cc2)cc1 ; c1ccc(-c2ccc3c(c2)c2ccccc2n3-c2cccc(-c3nc(-c4ccccc4)nc(-c4ccccc4)n3)c2)cc1]

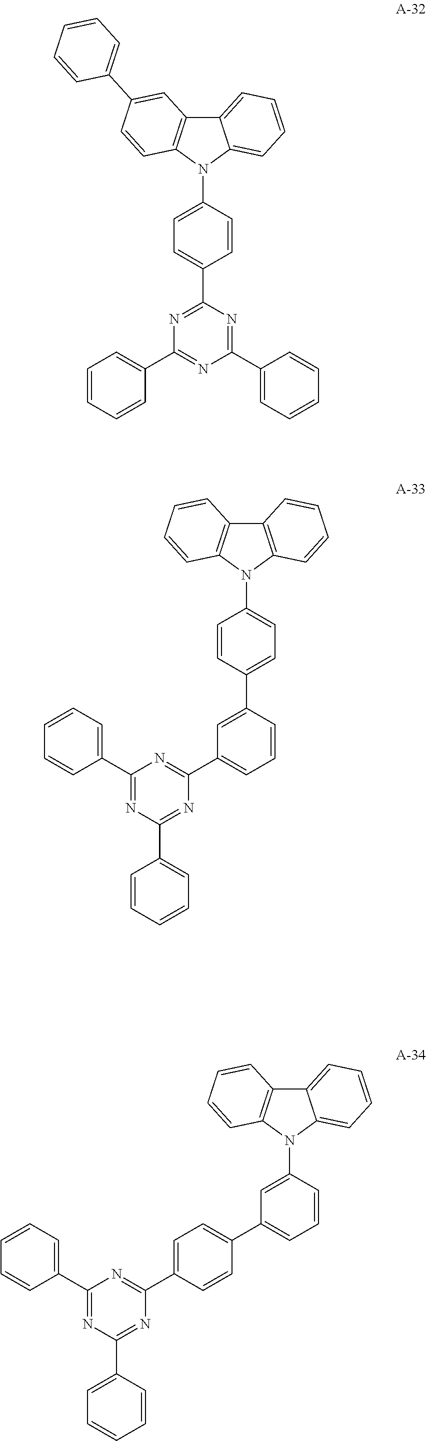 [SMILES: c1ccc(-c2ccc3c(c2)c2ccccc2n3-c2ccc(-c3nc(-c4ccccc4)nc(-c4ccccc4)n3)cc2)cc1 ; c1ccc(-c2nc(-c3ccccc3)nc(-c3cccc(-c4ccc(-n5c6ccccc6c6ccccc65)cc4)c3)n2)cc1 ; c1ccc(-c2nc(-c3ccccc3)nc(-c3ccc(-c4cccc(-n5c6ccccc6c6ccccc65)c4)cc3)n2)cc1]

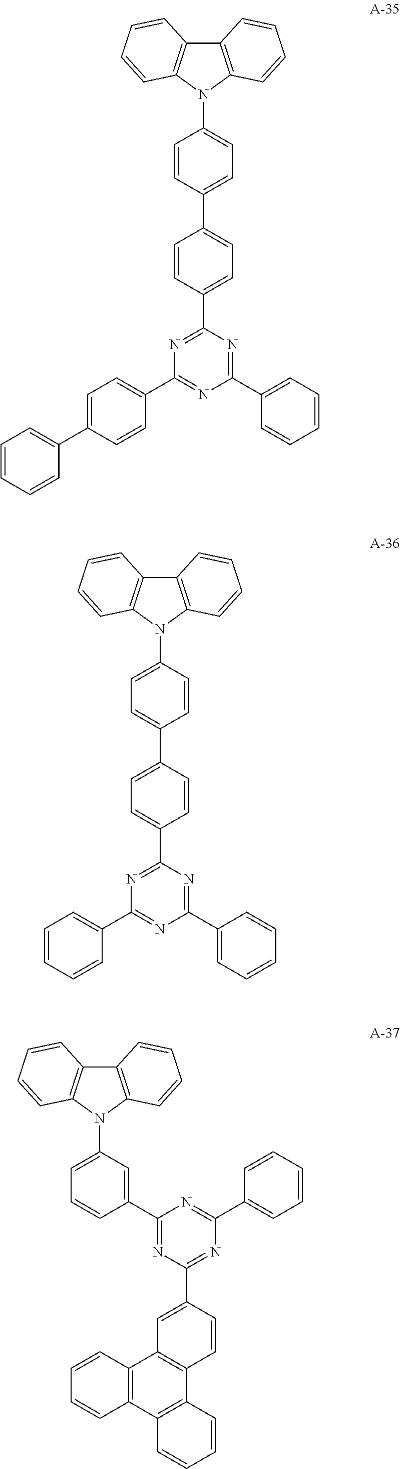 [SMILES: c1ccc(-c2ccc(-c3nc(-c4ccccc4)nc(-c4ccc(-c5ccc(-n6c7ccccc7c7ccccc76)cc5)cc4)n3)cc2)cc1 ; c1ccc(-c2nc(-c3ccccc3)nc(-c3ccc(-c4ccc(-n5c6ccccc6c6ccccc65)cc4)cc3)n2)cc1 ; c1ccc(-c2nc(-c3cccc(-n4c5ccccc5c5ccccc54)c3)nc(-c3ccc4c5ccccc5c5ccccc5c4c3)n2)cc1]

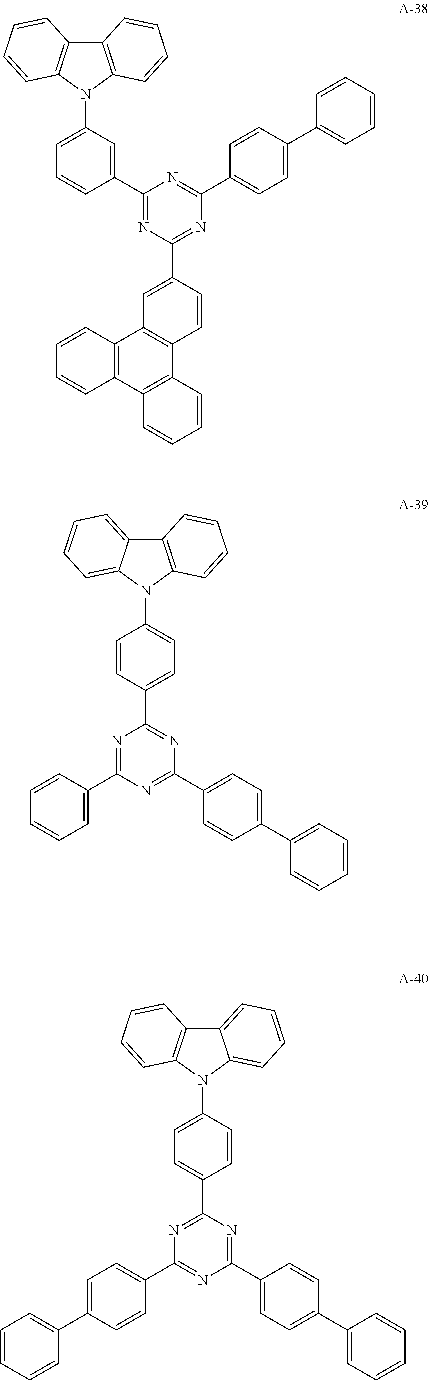 [SMILES: c1ccc(-c2ccc(-c3nc(-c4cccc(-n5c6ccccc6c6ccccc65)c4)nc(-c4ccc5c6ccccc6c6ccccc6c5c4)n3)cc2)cc1 ; c1ccc(-c2ccc(-c3nc(-c4ccccc4)nc(-c4ccc(-n5c6ccccc6c6ccccc65)cc4)n3)cc2)cc1 ; c1ccc(-c2ccc(-c3nc(-c4ccc(-c5ccccc5)cc4)nc(-c4ccc(-n5c6ccccc6c6ccccc65)cc4)n3)cc2)cc1]

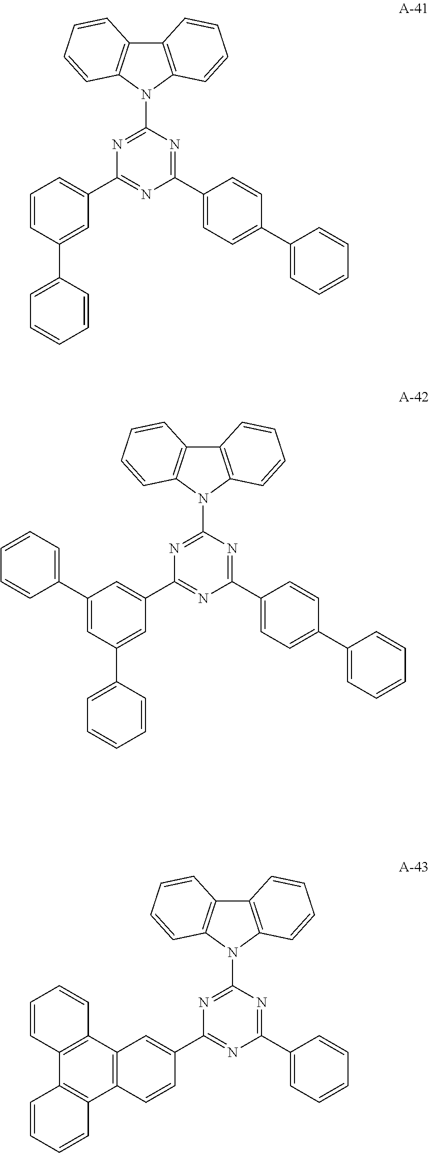 [SMILES: c1ccc(-c2ccc(-c3nc(-c4cccc(-c5ccccc5)c4)nc(-n4c5ccccc5c5ccccc54)n3)cc2)cc1 ; c1ccc(-c2ccc(-c3nc(-c4cc(-c5ccccc5)cc(-c5ccccc5)c4)nc(-n4c5ccccc5c5ccccc54)n3)cc2)cc1 ; c1ccc(-c2nc(-c3ccc4c5ccccc5c5ccccc5c4c3)nc(-n3c4ccccc4c4ccccc43)n2)cc1]

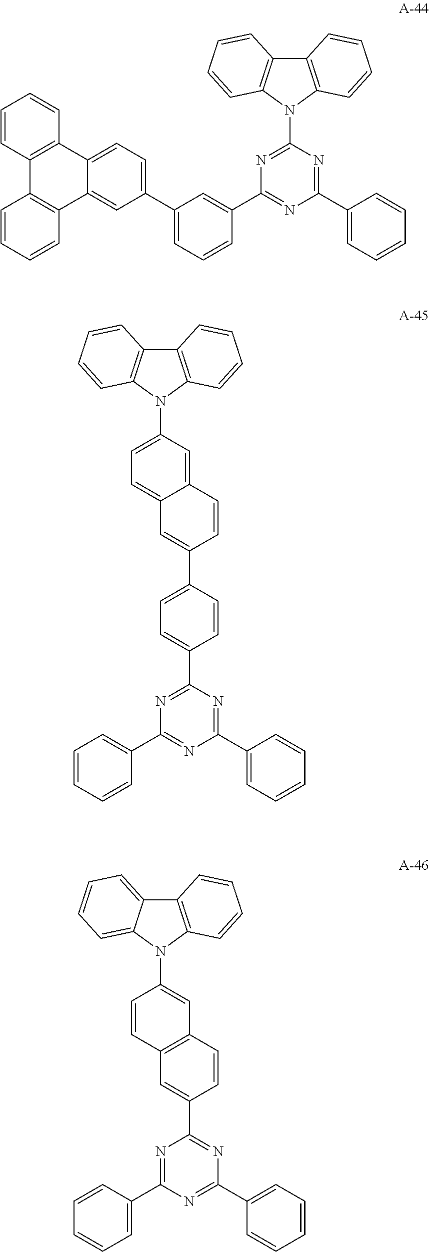 [SMILES: c1ccc(-c2nc(-c3cccc(-c4ccc5c6ccccc6c6ccccc6c5c4)c3)nc(-n3c4ccccc4c4ccccc43)n2)cc1 ; c1ccc(-c2nc(-c3ccccc3)nc(-c3ccc(-c4ccc5cc(-n6c7ccccc7c7ccccc76)ccc5c4)cc3)n2)cc1 ; c1ccc(-c2nc(-c3ccccc3)nc(-c3ccc4cc(-n5c6ccccc6c6ccccc65)ccc4c3)n2)cc1]

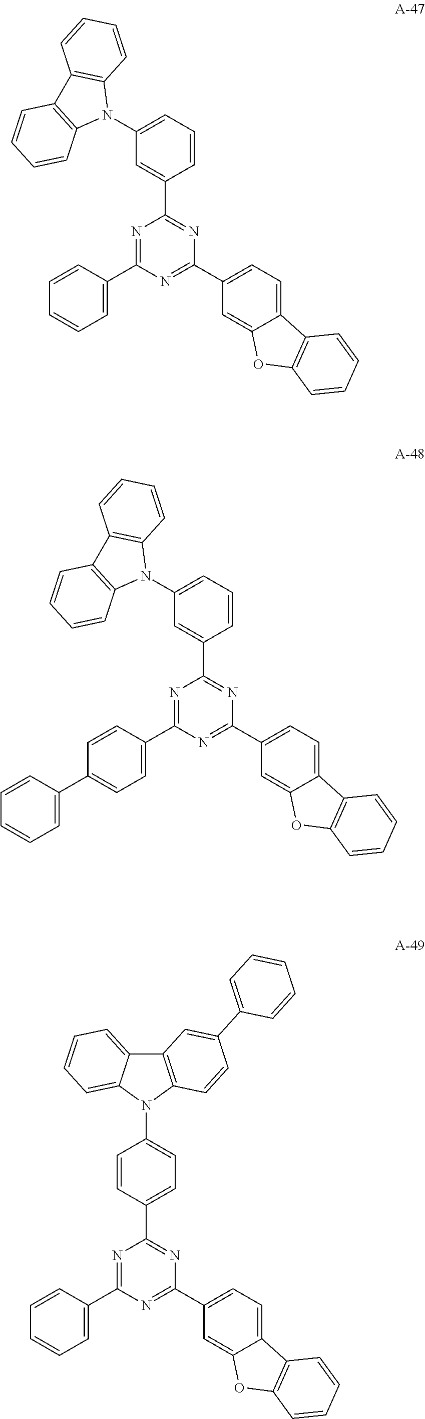 [SMILES: c1ccc(-c2nc(-c3cccc(-n4c5ccccc5c5ccccc54)c3)nc(-c3ccc4c(c3)oc3ccccc34)n2)cc1 ; c1ccc(-c2ccc(-c3nc(-c4cccc(-n5c6ccccc6c6ccccc65)c4)nc(-c4ccc5c(c4)oc4ccccc45)n3)cc2)cc1 ; c1ccc(-c2ccc3c(c2)c2ccccc2n3-c2ccc(-c3nc(-c4ccccc4)nc(-c4ccc5c(c4)oc4ccccc45)n3)cc2)cc1]

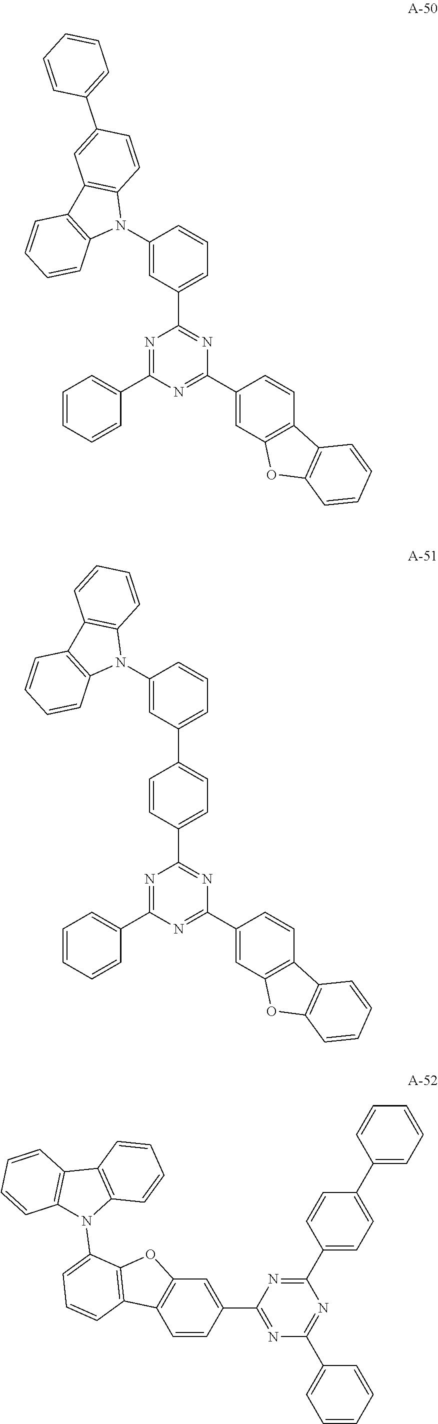 [SMILES: c1ccc(-c2ccc3c(c2)c2ccccc2n3-c2cccc(-c3nc(-c4ccccc4)nc(-c4ccc5c(c4)oc4ccccc45)n3)c2)cc1 ; c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2ccc(-c3nc(-c4ccccc4)nc(-c4ccc5c(c4)oc4c(-n6c7ccccc7c7ccccc76)cccc45)n3)cc2)cc1]

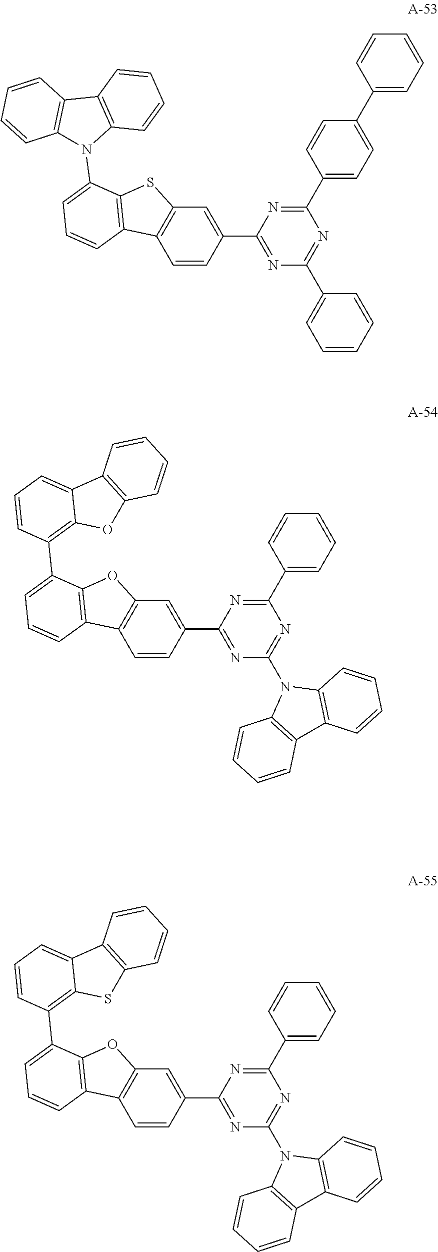 [SMILES: c1ccc(-c2ccc(-c3nc(-c4ccccc4)nc(-c4ccc5c(c4)sc4c(-n6c7ccccc7c7ccccc76)cccc45)n3)cc2)cc1 ; c1ccc(-c2nc(-c3ccc4c(c3)oc3c(-c5cccc6c5oc5ccccc56)cccc34)nc(-n3c4ccccc4c4ccccc43)n2)cc1 ; c1ccc(-c2nc(-c3ccc4c(c3)oc3c(-c5cccc6c5sc5ccccc56)cccc34)nc(-n3c4ccccc4c4ccccc43)n2)cc1]

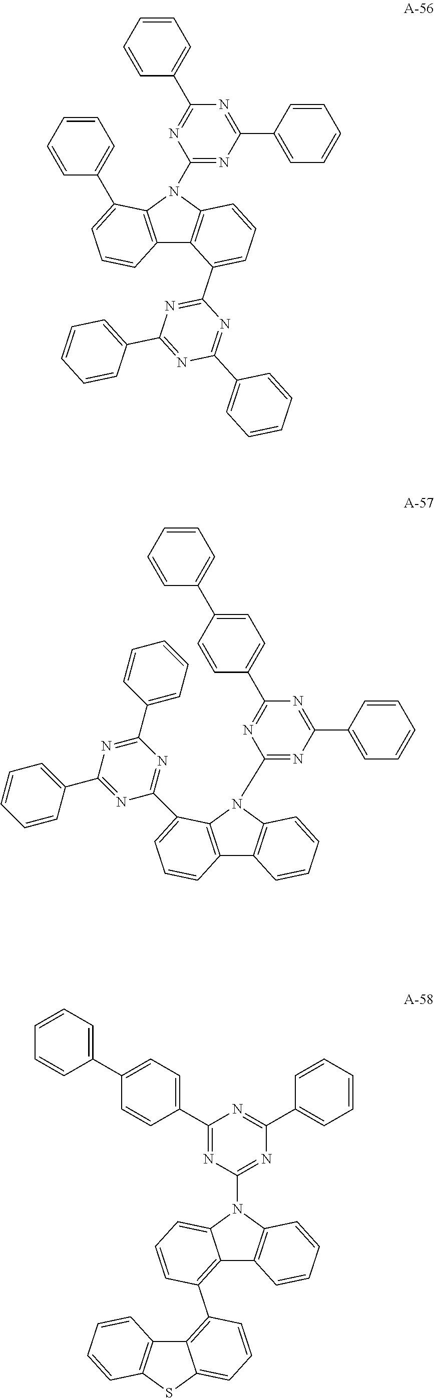 [SMILES: c1ccc(-c2nc(-c3ccccc3)nc(-c3cccc4c3c3cccc(-c5ccccc5)c3n4-c3nc(-c4ccccc4)nc(-c4ccccc4)n3)n2)cc1 ; c1ccc(-c2ccc(-c3nc(-c4ccccc4)nc(-n4c5ccccc5c5cccc(-c6nc(-c7ccccc7)nc(-c7ccccc7)n6)c54)n3)cc2)cc1 ; c1ccc(-c2ccc(-c3nc(-c4ccccc4)nc(-n4c5ccccc5c5c(-c6cccc7sc8ccccc8c67)cccc54)n3)cc2)cc1]

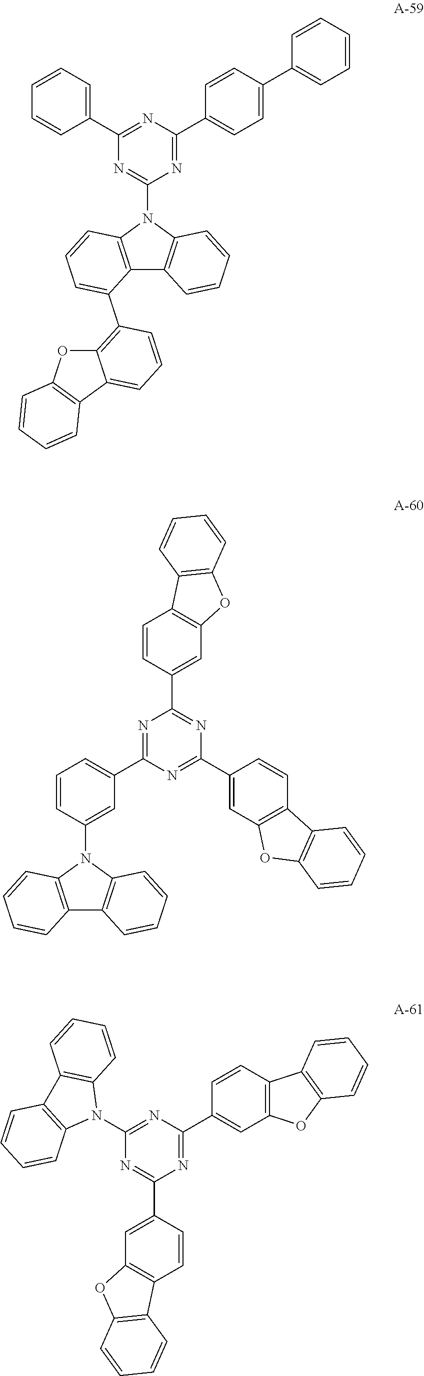 [SMILES: c1ccc(-c2ccc(-c3nc(-c4ccccc4)nc(-n4c5ccccc5c5c(-c6cccc7c6oc6ccccc67)cccc54)n3)cc2)cc1 ; c1cc(-c2nc(-c3ccc4c(c3)oc3ccccc34)nc(-c3ccc4c(c3)oc3ccccc34)n2)cc(-n2c3ccccc3c3ccccc32)c1 ; c1ccc2c(c1)oc1cc(-c3nc(-c4ccc5c(c4)oc4ccccc45)nc(-n4c5ccccc5c5ccccc54)n3)ccc12]

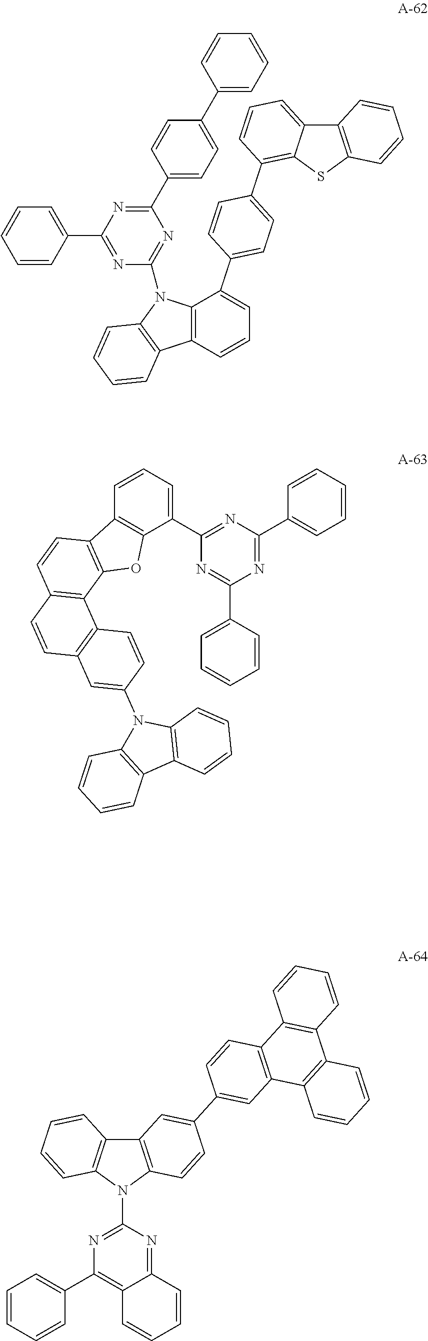 [SMILES: c1ccc(-c2ccc(-c3nc(-c4ccccc4)nc(-n4c5ccccc5c5cccc(-c6ccc(-c7cccc8c7sc7ccccc78)cc6)c54)n3)cc2)cc1 ; c1ccc(-c2nc(-c3ccccc3)nc(-c3cccc4c3oc3c4ccc4ccc5cc(-n6c7ccccc7c7ccccc76)ccc5c43)n2)cc1 ; c1ccc(-c2nc(-n3c4ccccc4c4cc(-c5ccc6c7ccccc7c7ccccc7c6c5)ccc43)nc3ccccc23)cc1]

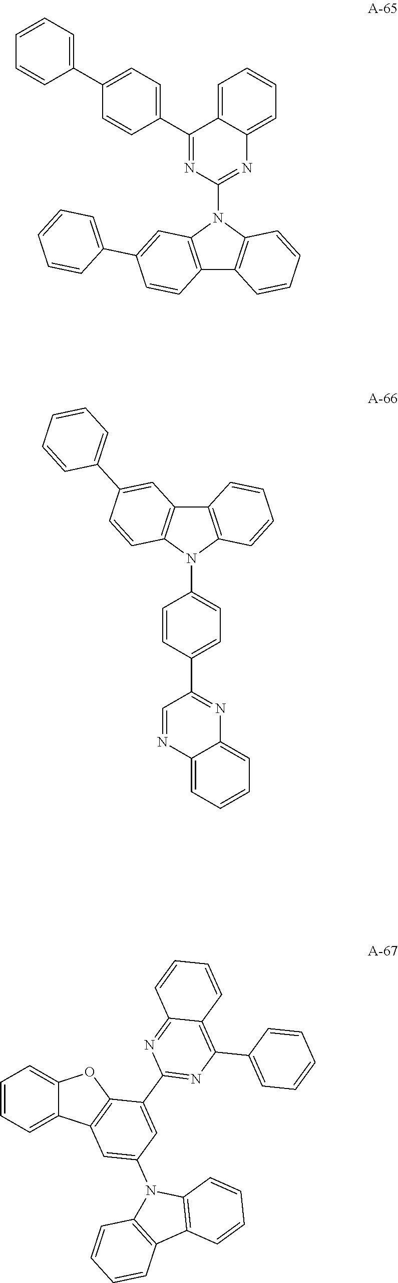 [SMILES: c1ccc(-c2ccc(-c3nc(-n4c5ccccc5c5ccc(-c6ccccc6)cc54)nc4ccccc34)cc2)cc1 ; c1ccc(-c2ccc3c(c2)c2ccccc2n3-c2ccc(-c3cnc4ccccc4n3)cc2)cc1 ; c1ccc(-c2nc(-c3cc(-n4c5ccccc5c5ccccc54)cc4c3oc3ccccc34)nc3ccccc23)cc1]

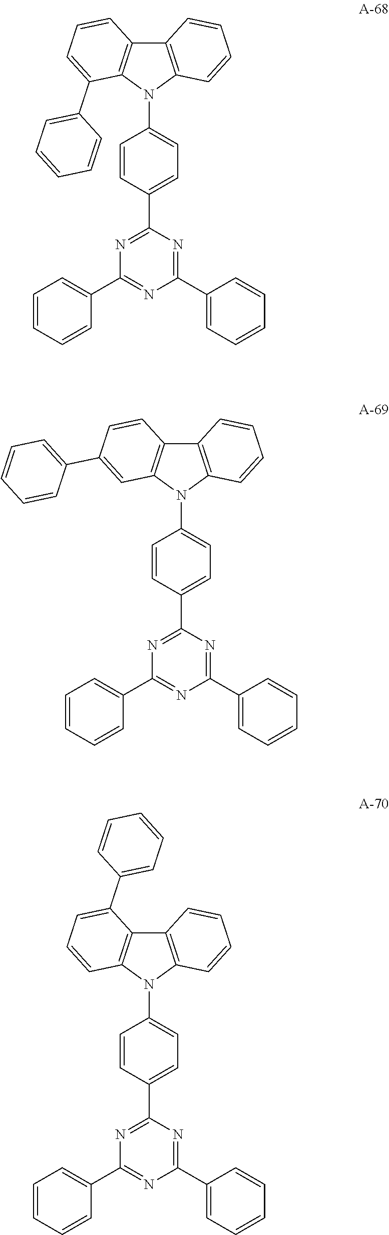 [SMILES: c1ccc(-c2nc(-c3ccccc3)nc(-c3ccc(-n4c5ccccc5c5cccc(-c6ccccc6)c54)cc3)n2)cc1 ; c1ccc(-c2ccc3c4ccccc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccccc5c5c(-c6ccccc6)cccc54)cc3)n2)cc1]

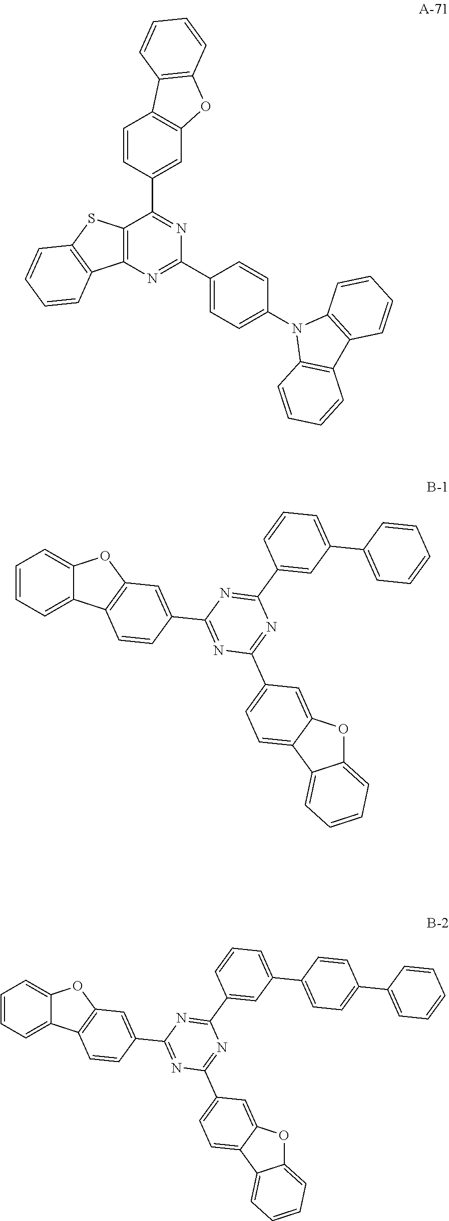 [SMILES: c1ccc2c(c1)oc1cc(-c3nc(-c4ccc(-n5c6ccccc6c6ccccc65)cc4)nc4c3sc3ccccc34)ccc12 ; c1ccc(-c2cccc(-c3nc(-c4ccc5c(c4)oc4ccccc45)nc(-c4ccc5c(c4)oc4ccccc45)n3)c2)cc1 ; c1ccc(-c2ccc(-c3cccc(-c4nc(-c5ccc6c(c5)oc5ccccc56)nc(-c5ccc6c(c5)oc5ccccc56)n4)c3)cc2)cc1]

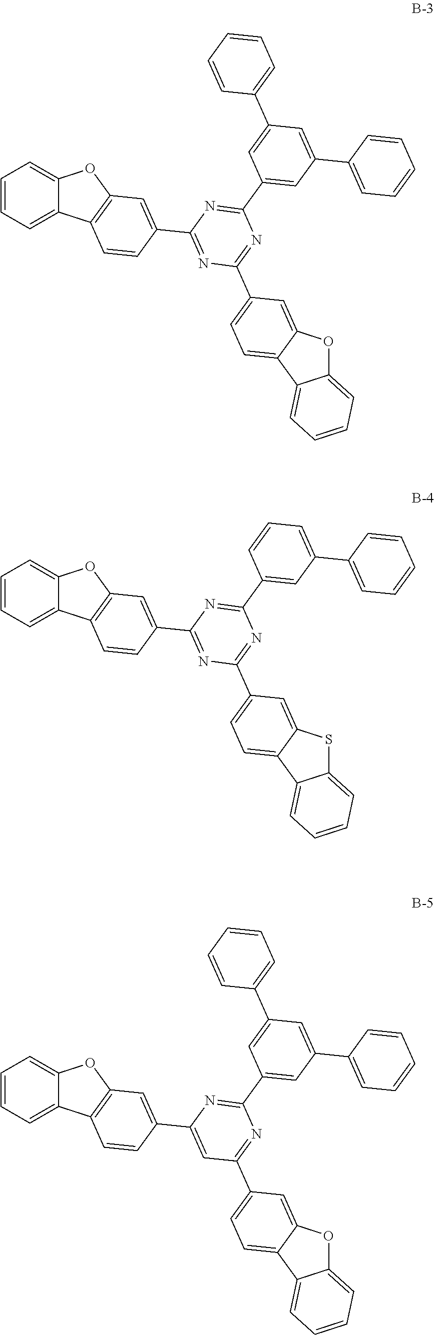 [SMILES: c1ccc(-c2cc(-c3ccccc3)cc(-c3nc(-c4ccc5c(c4)oc4ccccc45)nc(-c4ccc5c(c4)oc4ccccc45)n3)c2)cc1 ; c1ccc(-c2cccc(-c3nc(-c4ccc5c(c4)oc4ccccc45)nc(-c4ccc5c(c4)sc4ccccc45)n3)c2)cc1 ; c1ccc(-c2cc(-c3ccccc3)cc(-c3nc(-c4ccc5c(c4)oc4ccccc45)cc(-c4ccc5c(c4)oc4ccccc45)n3)c2)cc1]

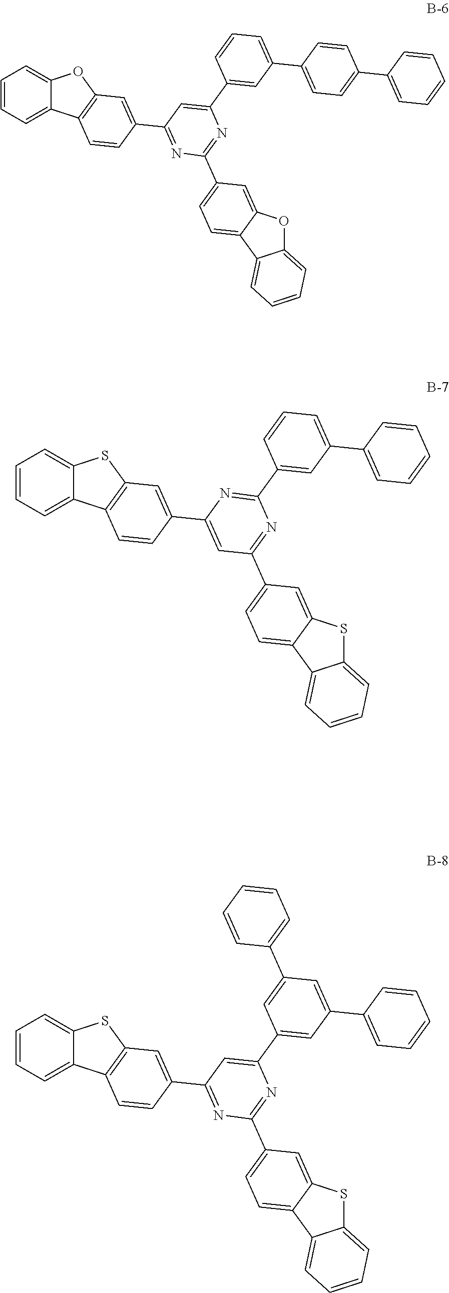 [SMILES: c1ccc(-c2ccc(-c3cccc(-c4cc(-c5ccc6c(c5)oc5ccccc56)nc(-c5ccc6c(c5)oc5ccccc56)n4)c3)cc2)cc1 ; c1ccc(-c2cccc(-c3nc(-c4ccc5c(c4)sc4ccccc45)cc(-c4ccc5c(c4)sc4ccccc45)n3)c2)cc1 ; c1ccc(-c2cc(-c3ccccc3)cc(-c3cc(-c4ccc5c(c4)sc4ccccc45)nc(-c4ccc5c(c4)sc4ccccc45)n3)c2)cc1]

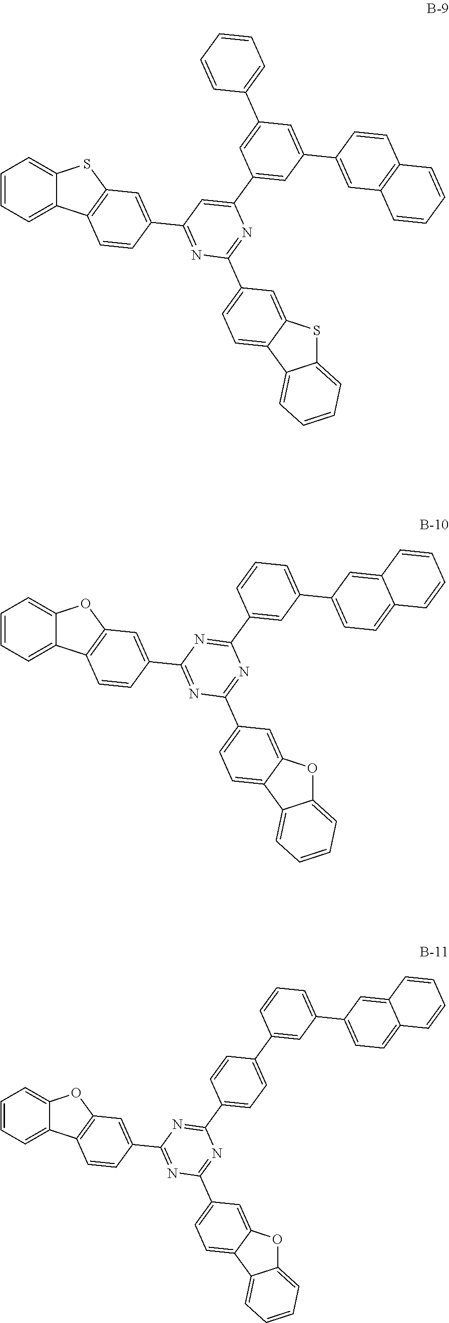 [SMILES: c1ccc(-c2cc(-c3ccc4ccccc4c3)cc(-c3cc(-c4ccc5c(c4)sc4ccccc45)nc(-c4ccc5c(c4)sc4ccccc45)n3)c2)cc1 ; c1cc(-c2ccc3ccccc3c2)cc(-c2nc(-c3ccc4c(c3)oc3ccccc34)nc(-c3ccc4c(c3)oc3ccccc34)n2)c1 ; c1cc(-c2ccc(-c3nc(-c4ccc5c(c4)oc4ccccc45)nc(-c4ccc5c(c4)oc4ccccc45)n3)cc2)cc(-c2ccc3ccccc3c2)c1]

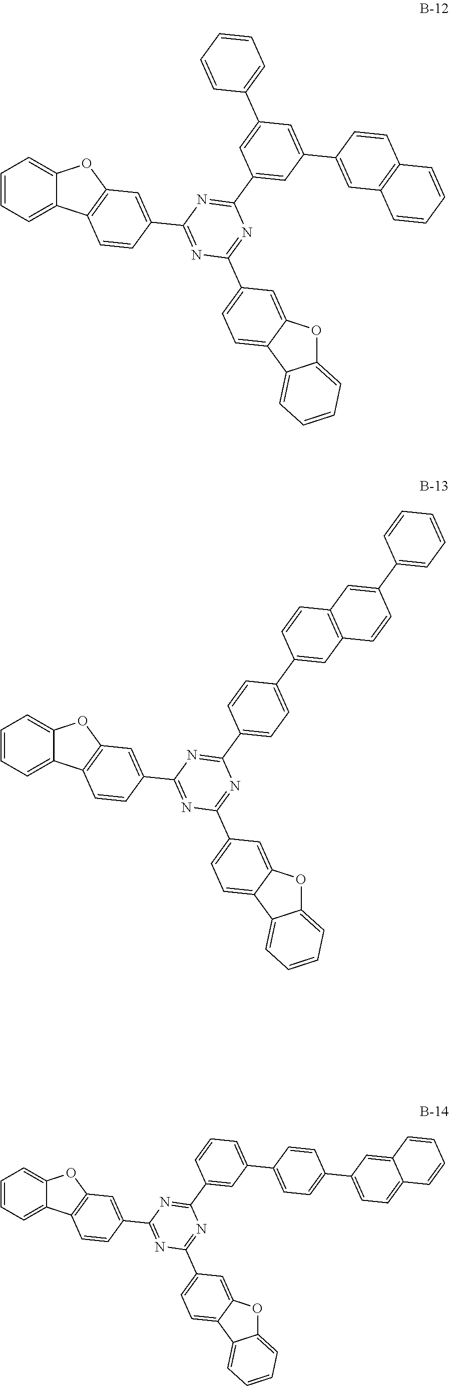 [SMILES: c1ccc(-c2cc(-c3ccc4ccccc4c3)cc(-c3nc(-c4ccc5c(c4)oc4ccccc45)nc(-c4ccc5c(c4)oc4ccccc45)n3)c2)cc1 ; c1ccc(-c2ccc3cc(-c4ccc(-c5nc(-c6ccc7c(c6)oc6ccccc67)nc(-c6ccc7c(c6)oc6ccccc67)n5)cc4)ccc3c2)cc1 ; c1cc(-c2ccc(-c3ccc4ccccc4c3)cc2)cc(-c2nc(-c3ccc4c(c3)oc3ccccc34)nc(-c3ccc4c(c3)oc3ccccc34)n2)c1]

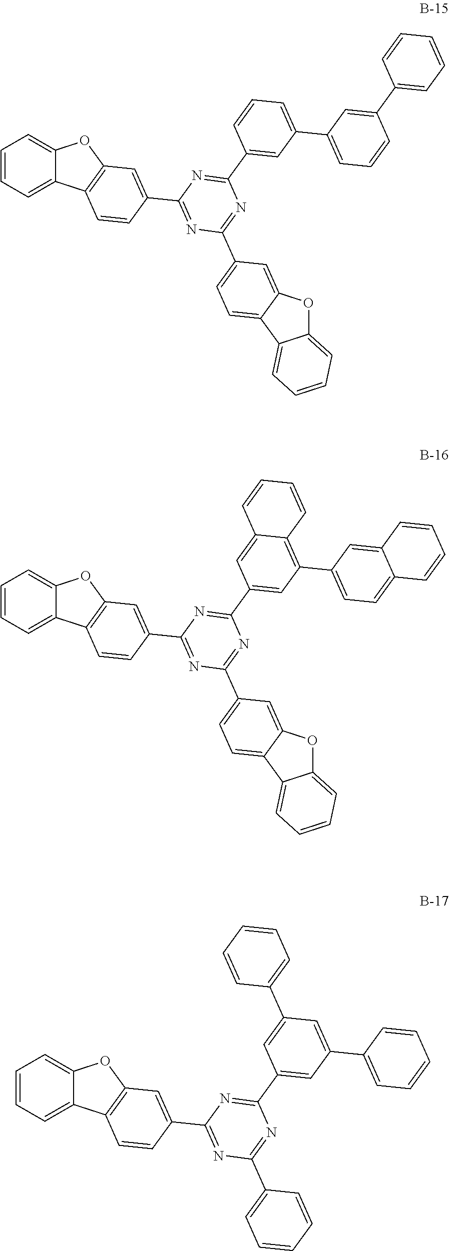 [SMILES: c1ccc(-c2cccc(-c3cccc(-c4nc(-c5ccc6c(c5)oc5ccccc56)nc(-c5ccc6c(c5)oc5ccccc56)n4)c3)c2)cc1 ; c1ccc2cc(-c3cc(-c4nc(-c5ccc6c(c5)oc5ccccc56)nc(-c5ccc6c(c5)oc5ccccc56)n4)cc4ccccc34)ccc2c1 ; c1ccc(-c2cc(-c3ccccc3)cc(-c3nc(-c4ccccc4)nc(-c4ccc5c(c4)oc4ccccc45)n3)c2)cc1]

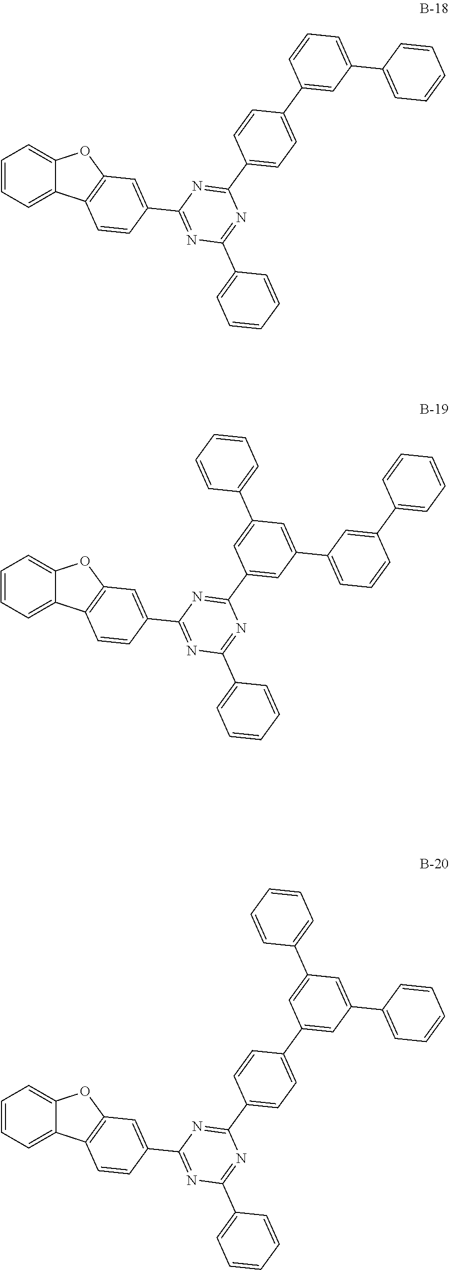 [SMILES: c1ccc(-c2cccc(-c3ccc(-c4nc(-c5ccccc5)nc(-c5ccc6c(c5)oc5ccccc56)n4)cc3)c2)cc1 ; c1ccc(-c2cccc(-c3cc(-c4ccccc4)cc(-c4nc(-c5ccccc5)nc(-c5ccc6c(c5)oc5ccccc56)n4)c3)c2)cc1 ; c1ccc(-c2cc(-c3ccccc3)cc(-c3ccc(-c4nc(-c5ccccc5)nc(-c5ccc6c(c5)oc5ccccc56)n4)cc3)c2)cc1]

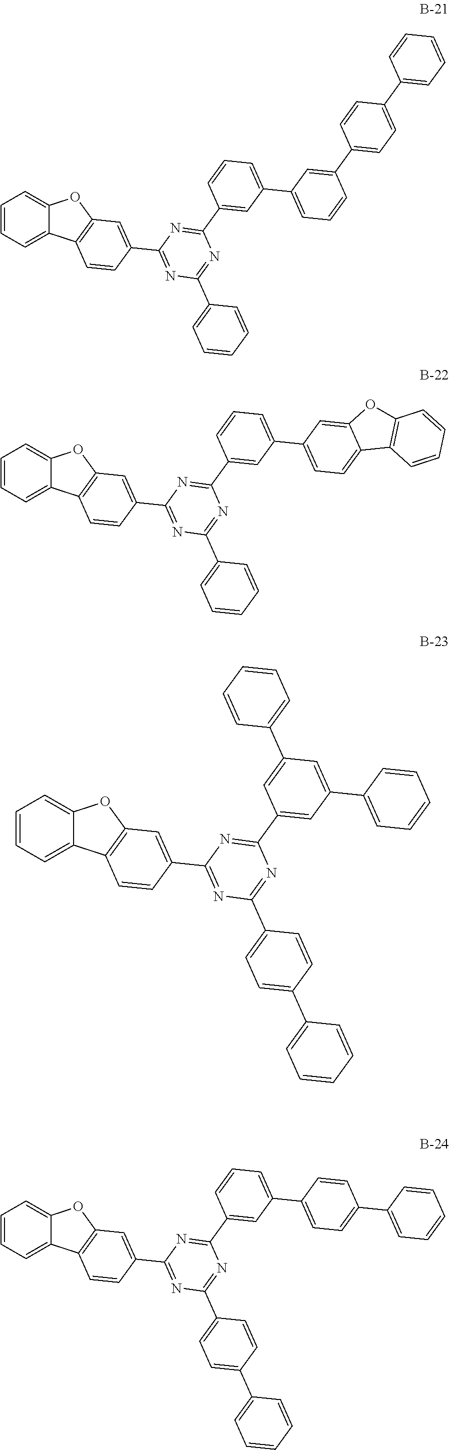 [SMILES: c1ccc(-c2ccc(-c3cccc(-c4cccc(-c5nc(-c6ccccc6)nc(-c6ccc7c(c6)oc6ccccc67)n5)c4)c3)cc2)cc1 ; c1ccc(-c2nc(-c3cccc(-c4ccc5c(c4)oc4ccccc45)c3)nc(-c3ccc4c(c3)oc3ccccc34)n2)cc1 ; c1ccc(-c2ccc(-c3nc(-c4cc(-c5ccccc5)cc(-c5ccccc5)c4)nc(-c4ccc5c(c4)oc4ccccc45)n3)cc2)cc1 ; c1ccc(-c2ccc(-c3cccc(-c4nc(-c5ccc(-c6ccccc6)cc5)nc(-c5ccc6c(c5)oc5ccccc56)n4)c3)cc2)cc1]

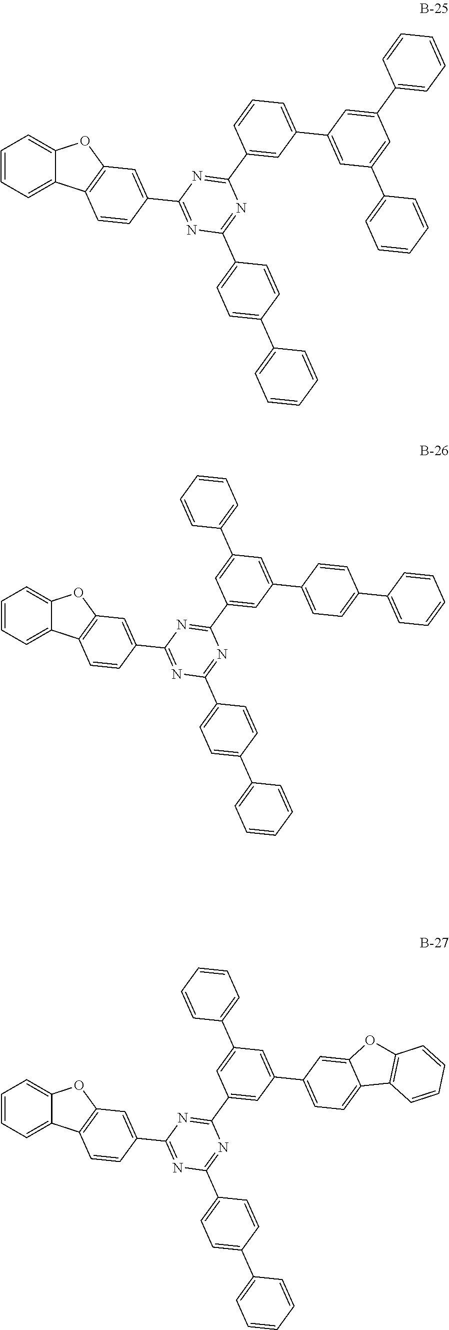 [SMILES: c1ccc(-c2ccc(-c3nc(-c4cccc(-c5cc(-c6ccccc6)cc(-c6ccccc6)c5)c4)nc(-c4ccc5c(c4)oc4ccccc45)n3)cc2)cc1 ; c1ccc(-c2ccc(-c3cc(-c4ccccc4)cc(-c4nc(-c5ccc(-c6ccccc6)cc5)nc(-c5ccc6c(c5)oc5ccccc56)n4)c3)cc2)cc1 ; c1ccc(-c2ccc(-c3nc(-c4cc(-c5ccccc5)cc(-c5ccc6c(c5)oc5ccccc56)c4)nc(-c4ccc5c(c4)oc4ccccc45)n3)cc2)cc1]

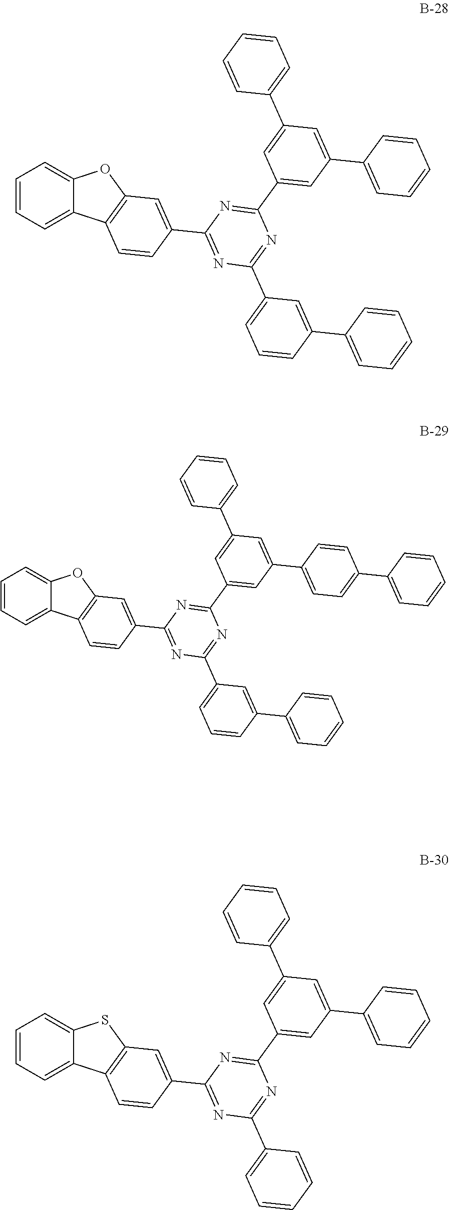 [SMILES: c1ccc(-c2cccc(-c3nc(-c4cc(-c5ccccc5)cc(-c5ccccc5)c4)nc(-c4ccc5c(c4)oc4ccccc45)n3)c2)cc1 ; c1ccc(-c2ccc(-c3cc(-c4ccccc4)cc(-c4nc(-c5cccc(-c6ccccc6)c5)nc(-c5ccc6c(c5)oc5ccccc56)n4)c3)cc2)cc1 ; c1ccc(-c2cc(-c3ccccc3)cc(-c3nc(-c4ccccc4)nc(-c4ccc5c(c4)sc4ccccc45)n3)c2)cc1]

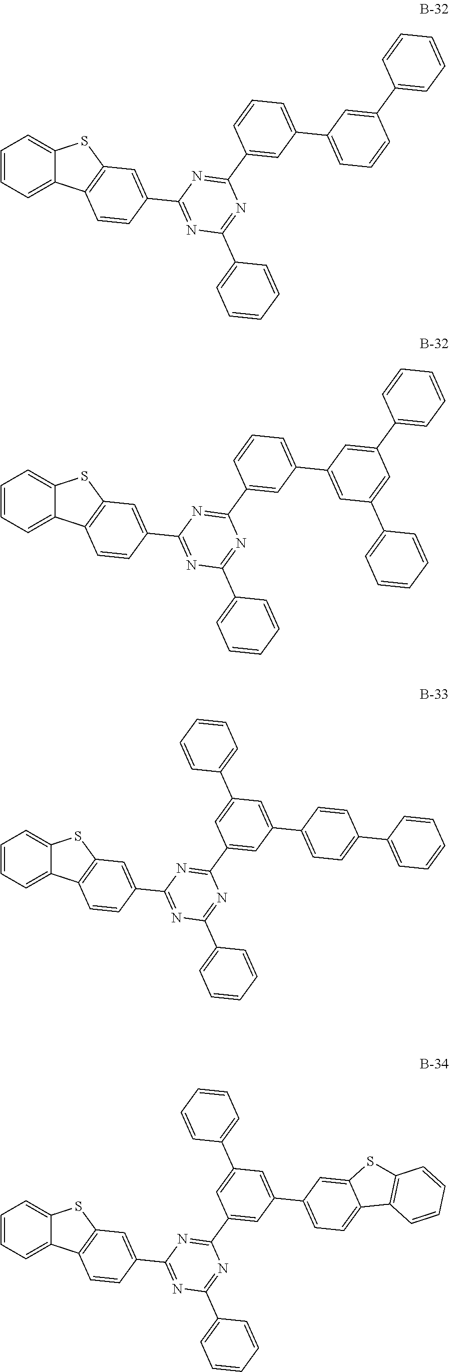 [SMILES: c1ccc(-c2cccc(-c3cccc(-c4nc(-c5ccccc5)nc(-c5ccc6c(c5)sc5ccccc56)n4)c3)c2)cc1 ; c1ccc(-c2cc(-c3ccccc3)cc(-c3cccc(-c4nc(-c5ccccc5)nc(-c5ccc6c(c5)sc5ccccc56)n4)c3)c2)cc1 ; c1ccc(-c2ccc(-c3cc(-c4ccccc4)cc(-c4nc(-c5ccccc5)nc(-c5ccc6c(c5)sc5ccccc56)n4)c3)cc2)cc1 ; c1ccc(-c2cc(-c3ccc4c(c3)sc3ccccc34)cc(-c3nc(-c4ccccc4)nc(-c4ccc5c(c4)sc4ccccc45)n3)c2)cc1]

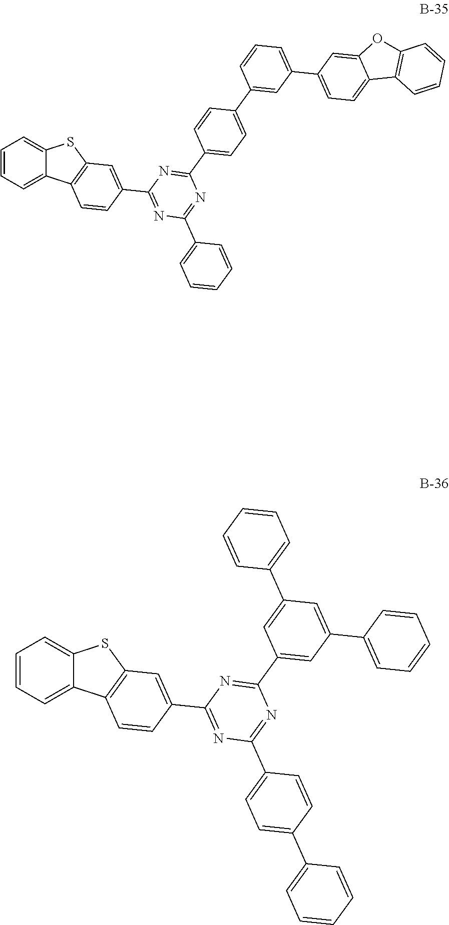 [SMILES: c1ccc(-c2nc(-c3ccc(-c4cccc(-c5ccc6c(c5)oc5ccccc56)c4)cc3)nc(-c3ccc4c(c3)sc3ccccc34)n2)cc1 ; c1ccc(-c2ccc(-c3nc(-c4cc(-c5ccccc5)cc(-c5ccccc5)c4)nc(-c4ccc5c(c4)sc4ccccc45)n3)cc2)cc1]

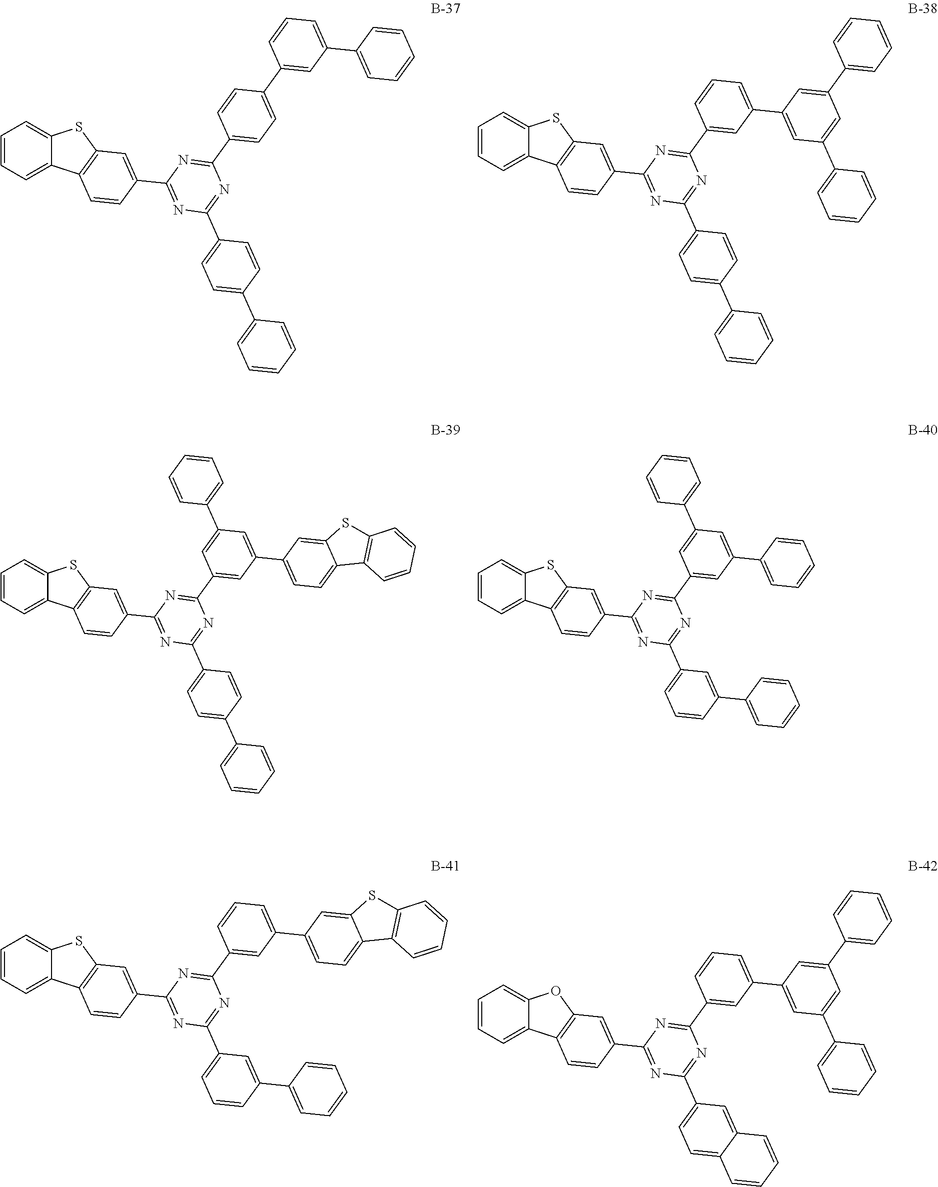

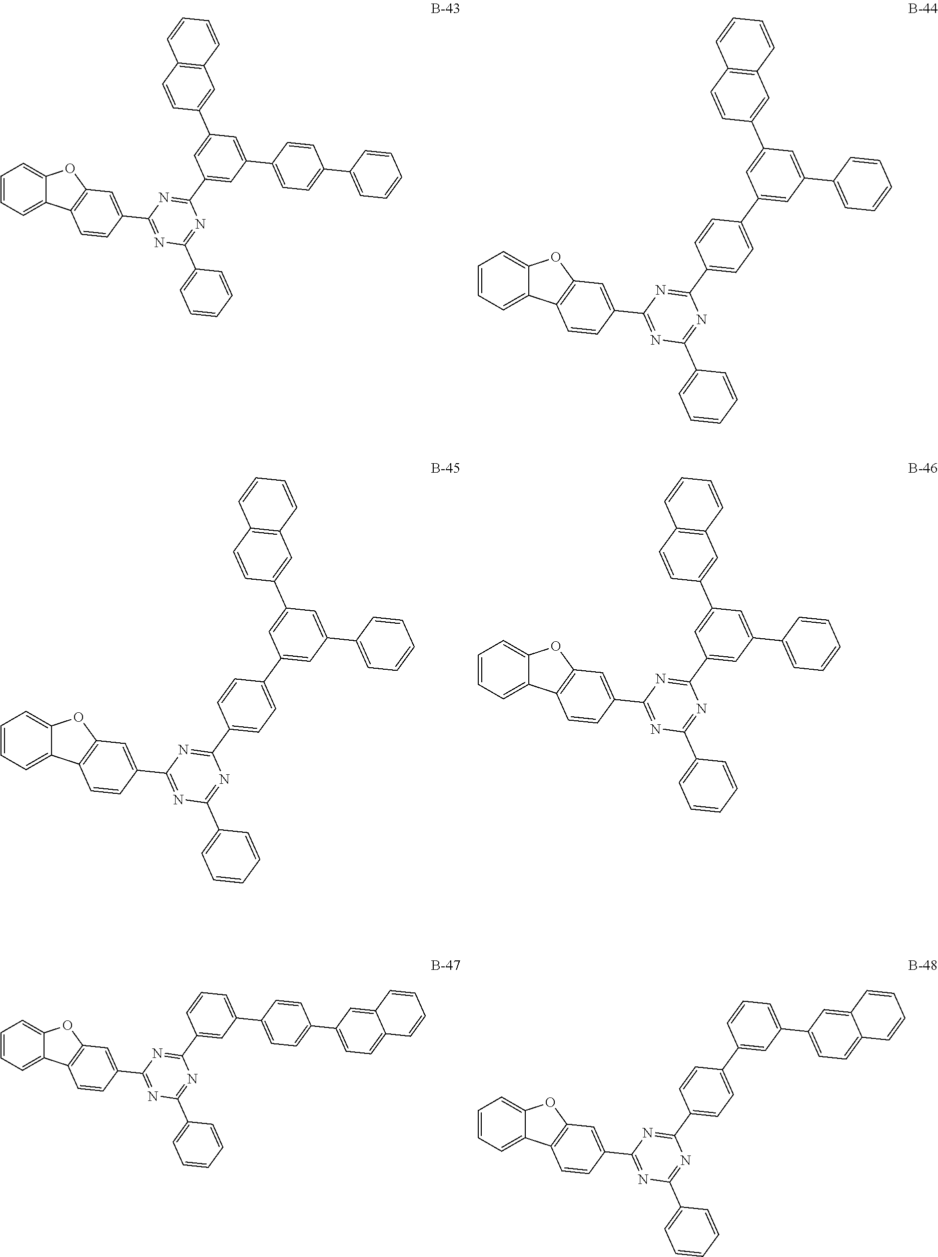

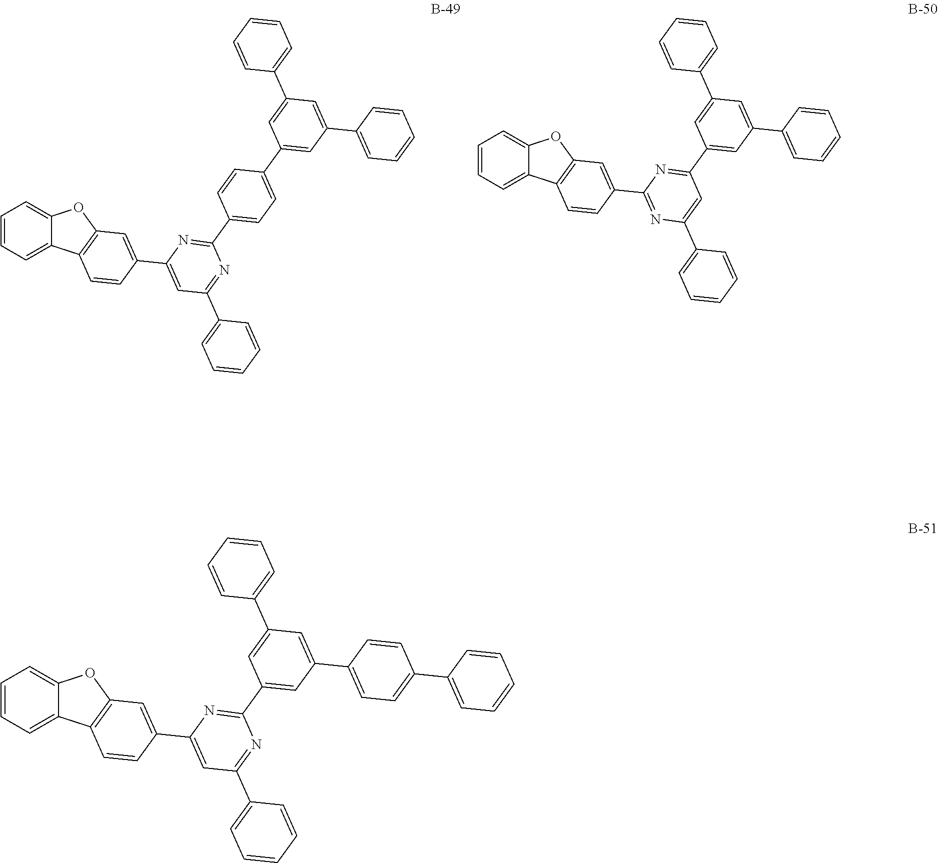 [SMILES: c1ccc(-c2cc(-c3ccccc3)cc(-c3ccc(-c4nc(-c5ccccc5)cc(-c5ccc6c(c5)oc5ccccc56)n4)cc3)c2)cc1 ; c1ccc(-c2cc(-c3ccccc3)cc(-c3cc(-c4ccccc4)nc(-c4ccc5c(c4)oc4ccccc45)n3)c2)cc1 ; c1ccc(-c2ccc(-c3cc(-c4ccccc4)cc(-c4nc(-c5ccccc5)cc(-c5ccc6c(c5)oc5ccccc56)n4)c3)cc2)cc1]

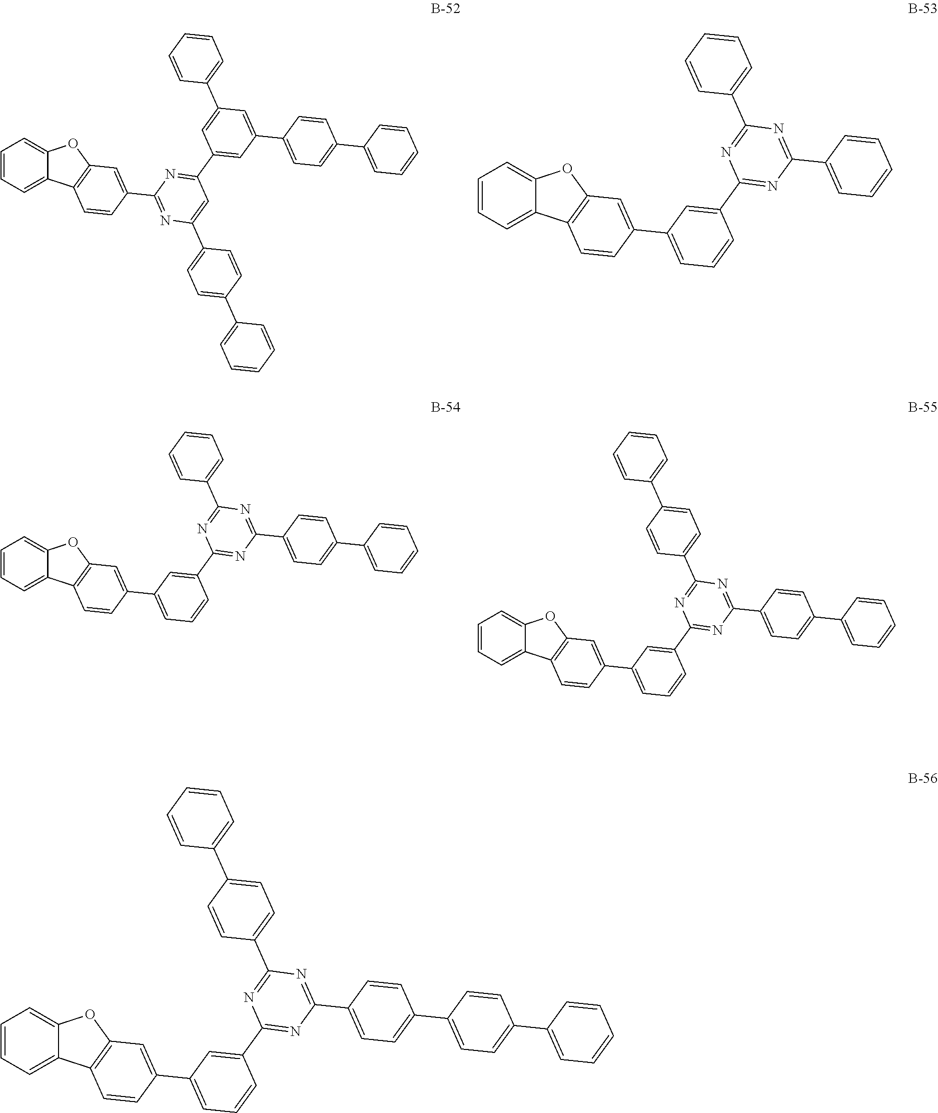 [SMILES: c1ccc(-c2ccc(-c3cc(-c4ccccc4)cc(-c4cc(-c5ccc(-c6ccccc6)cc5)nc(-c5ccc6c(c5)oc5ccccc56)n4)c3)cc2)cc1 ; c1ccc(-c2nc(-c3ccccc3)nc(-c3cccc(-c4ccc5c(c4)oc4ccccc45)c3)n2)cc1 ; c1ccc(-c2ccc(-c3nc(-c4ccccc4)nc(-c4cccc(-c5ccc6c(c5)oc5ccccc56)c4)n3)cc2)cc1 ; c1ccc(-c2ccc(-c3nc(-c4ccc(-c5ccccc5)cc4)nc(-c4cccc(-c5ccc6c(c5)oc5ccccc56)c4)n3)cc2)cc1 ; c1ccc(-c2ccc(-c3ccc(-c4nc(-c5ccc(-c6ccccc6)cc5)nc(-c5cccc(-c6ccc7c(c6)oc6ccccc67)c5)n4)cc3)cc2)cc1]

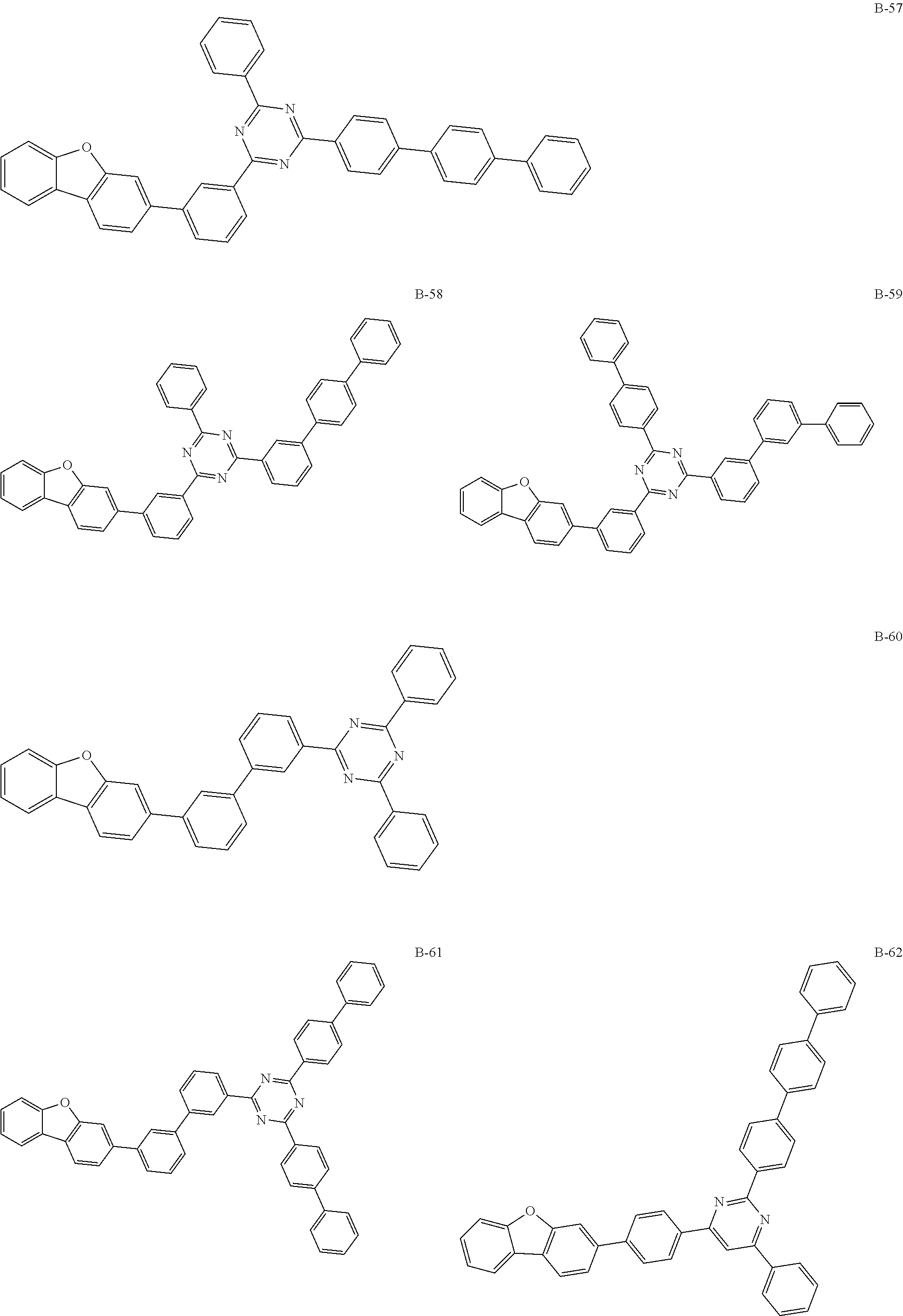

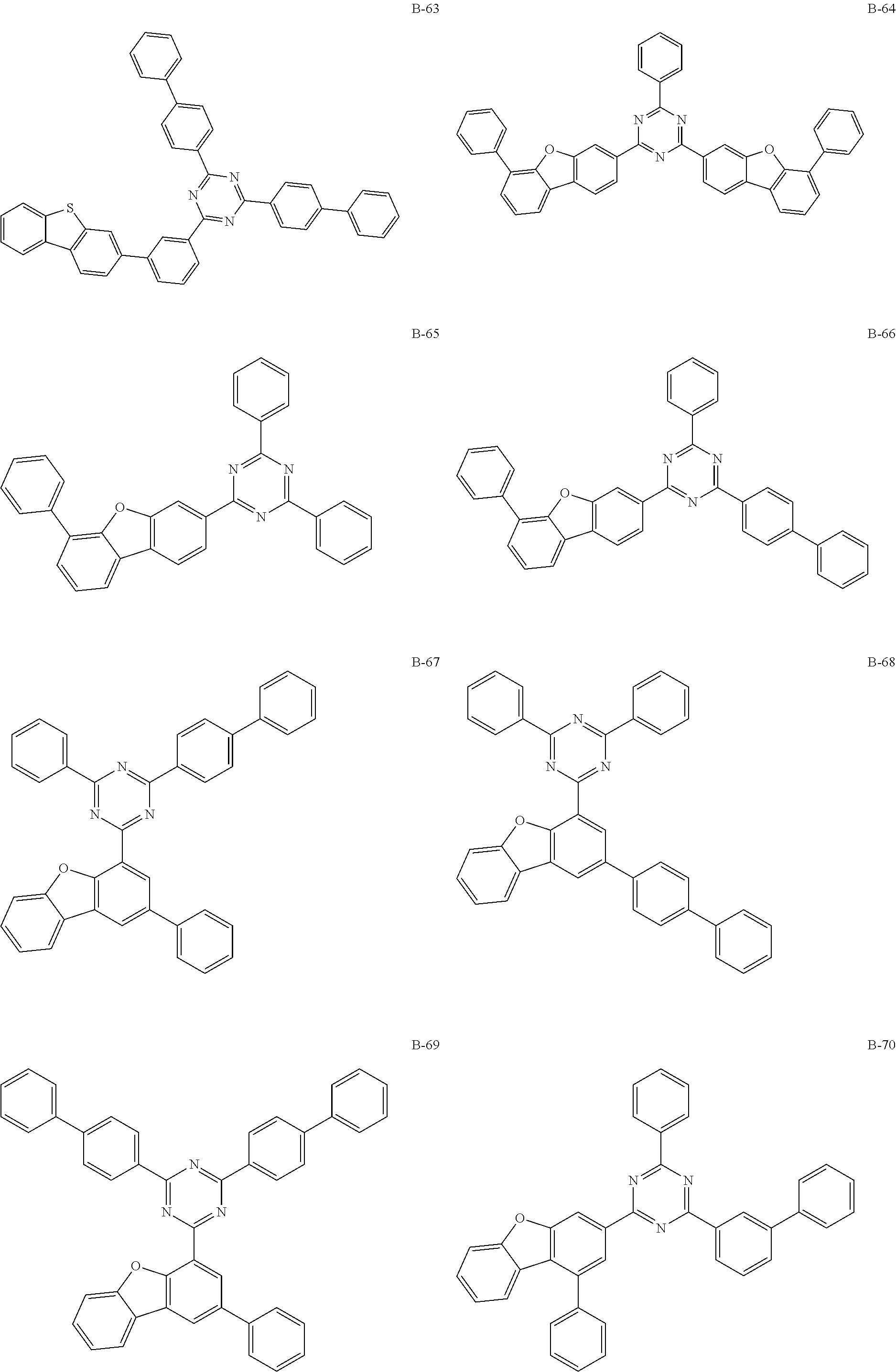

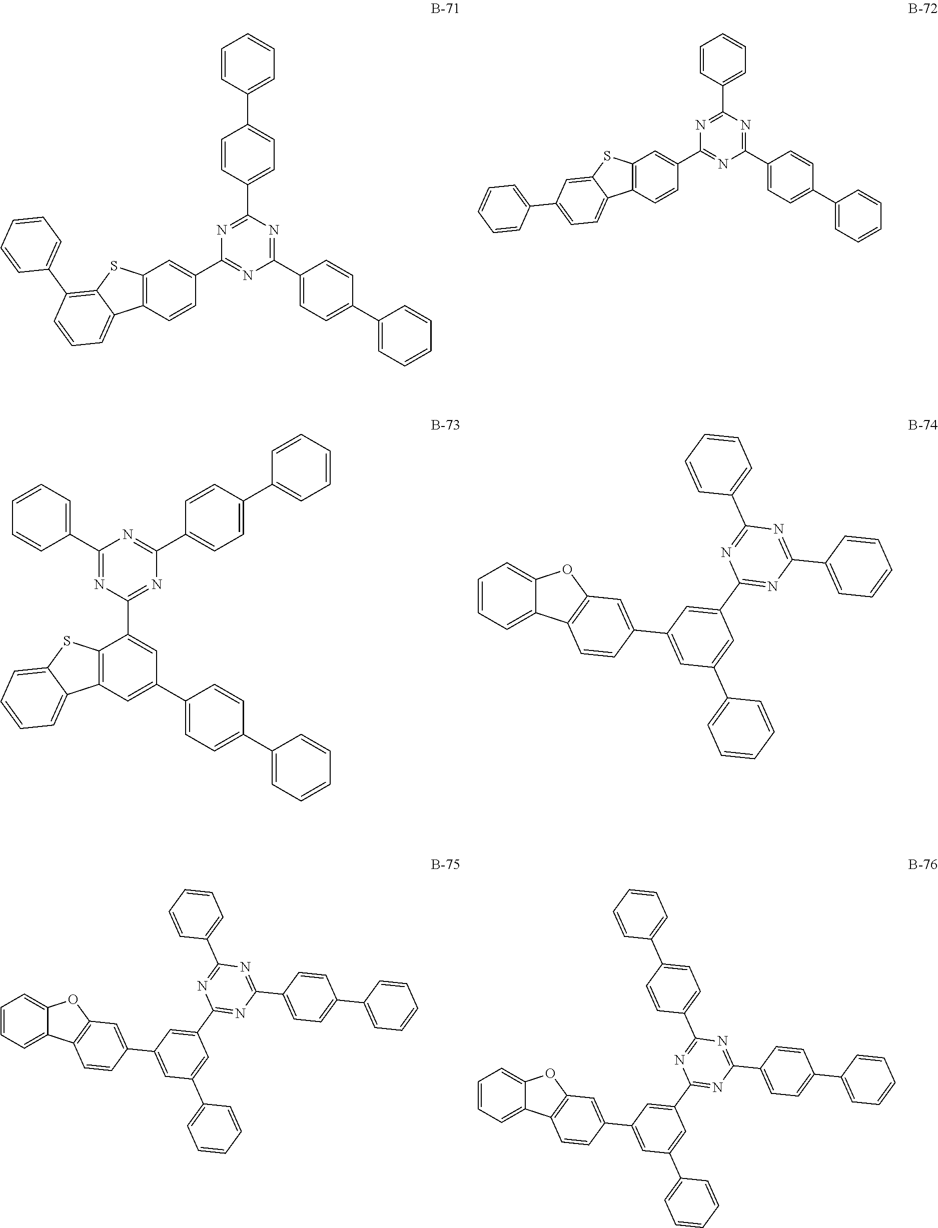

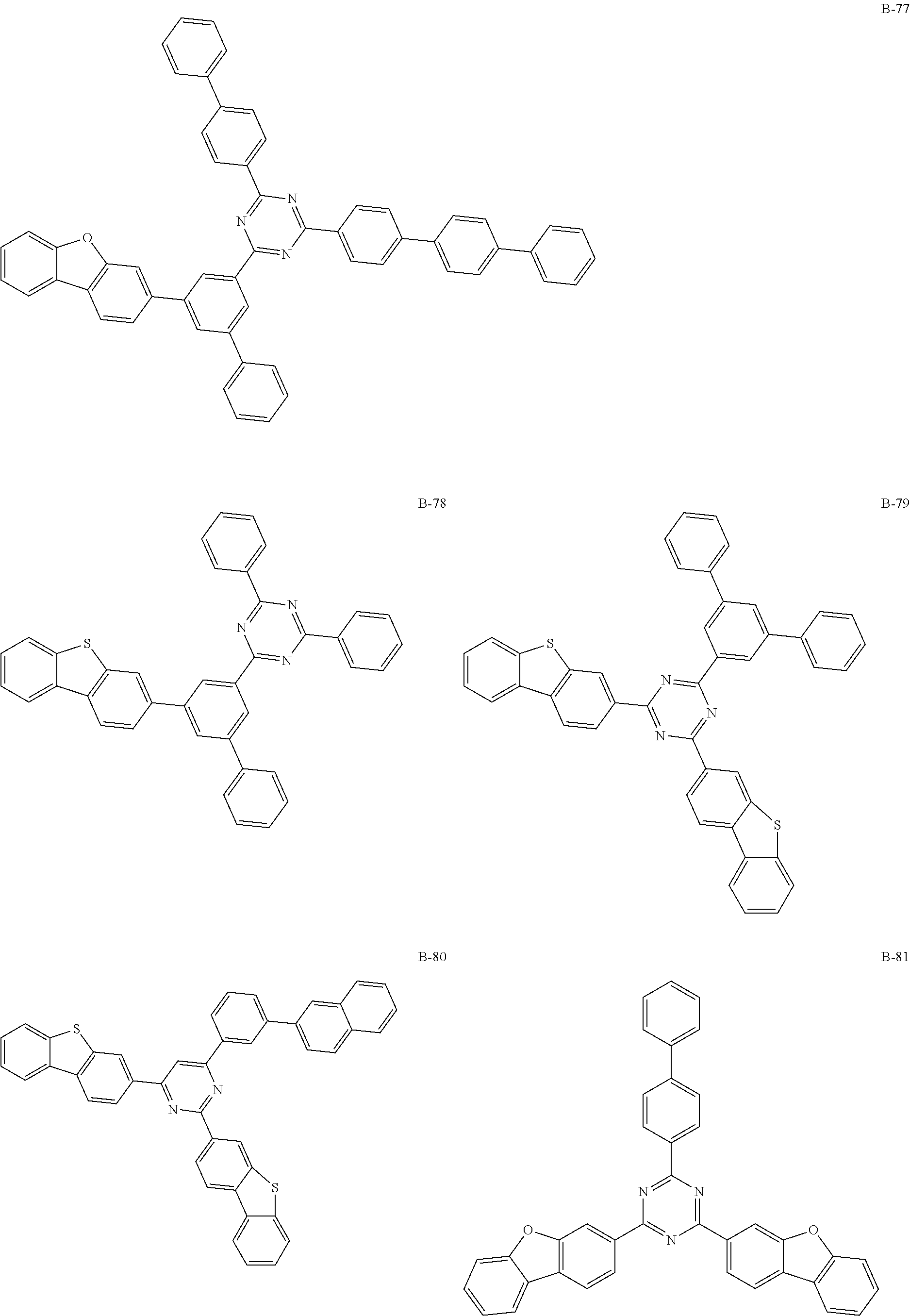 [SMILES: c1ccc(-c2ccc(-c3ccc(-c4nc(-c5ccc(-c6ccccc6)cc5)nc(-c5cc(-c6ccccc6)cc(-c6ccc7c(c6)oc6ccccc67)c5)n4)cc3)cc2)cc1 ; c1ccc(-c2cc(-c3ccc4c(c3)sc3ccccc34)cc(-c3nc(-c4ccccc4)nc(-c4ccccc4)n3)c2)cc1 ; c1ccc(-c2cc(-c3ccccc3)cc(-c3nc(-c4ccc5c(c4)sc4ccccc45)nc(-c4ccc5c(c4)sc4ccccc45)n3)c2)cc1 ; c1cc(-c2ccc3ccccc3c2)cc(-c2cc(-c3ccc4c(c3)sc3ccccc34)nc(-c3ccc4c(c3)sc3ccccc34)n2)c1 ; c1ccc(-c2ccc(-c3nc(-c4ccc5c(c4)oc4ccccc45)nc(-c4ccc5c(c4)oc4ccccc45)n3)cc2)cc1]

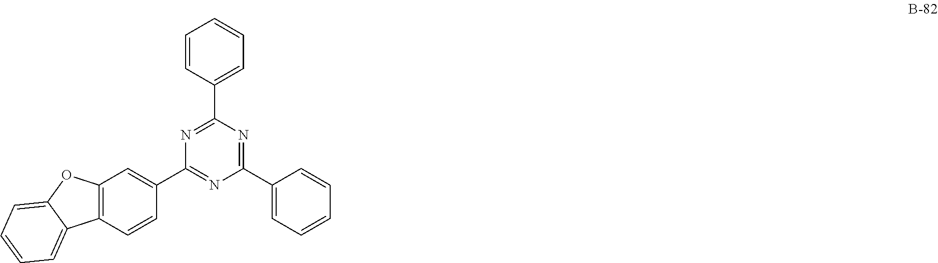 [SMILES: c1ccc(-c2nc(-c3ccccc3)nc(-c3ccc4c(c3)oc3ccccc34)n2)cc1]

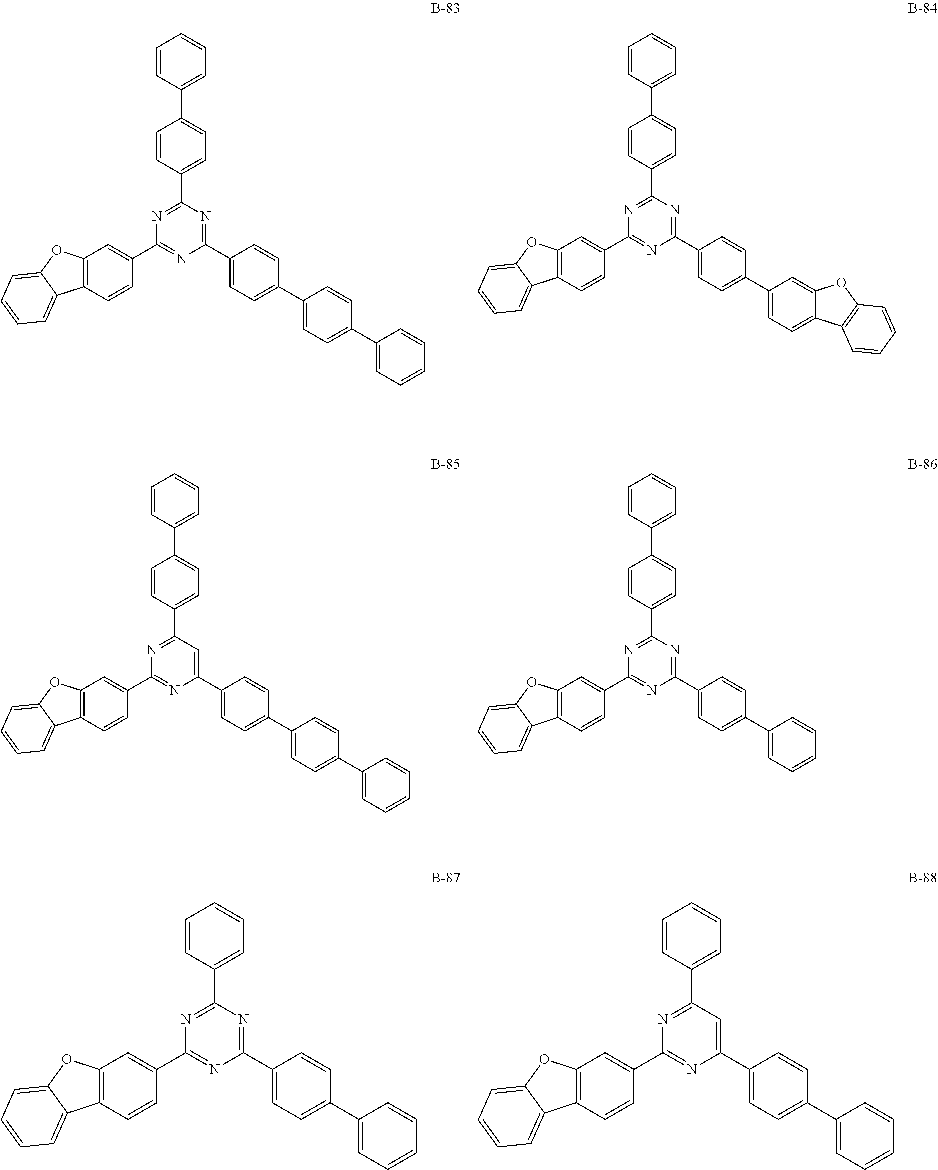 [SMILES: c1ccc(-c2ccc(-c3ccc(-c4nc(-c5ccc(-c6ccccc6)cc5)nc(-c5ccc6c(c5)oc5ccccc56)n4)cc3)cc2)cc1 ; c1ccc(-c2ccc(-c3nc(-c4ccc(-c5ccc6c(c5)oc5ccccc56)cc4)nc(-c4ccc5c(c4)oc4ccccc45)n3)cc2)cc1 ; c1ccc(-c2ccc(-c3ccc(-c4cc(-c5ccc(-c6ccccc6)cc5)nc(-c5ccc6c(c5)oc5ccccc56)n4)cc3)cc2)cc1 ; c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccccc4)nc(-c4ccc5c(c4)oc4ccccc45)n3)cc2)cc1 ; c1ccc(-c2ccc(-c3cc(-c4ccccc4)nc(-c4ccc5c(c4)oc4ccccc45)n3)cc2)cc1]

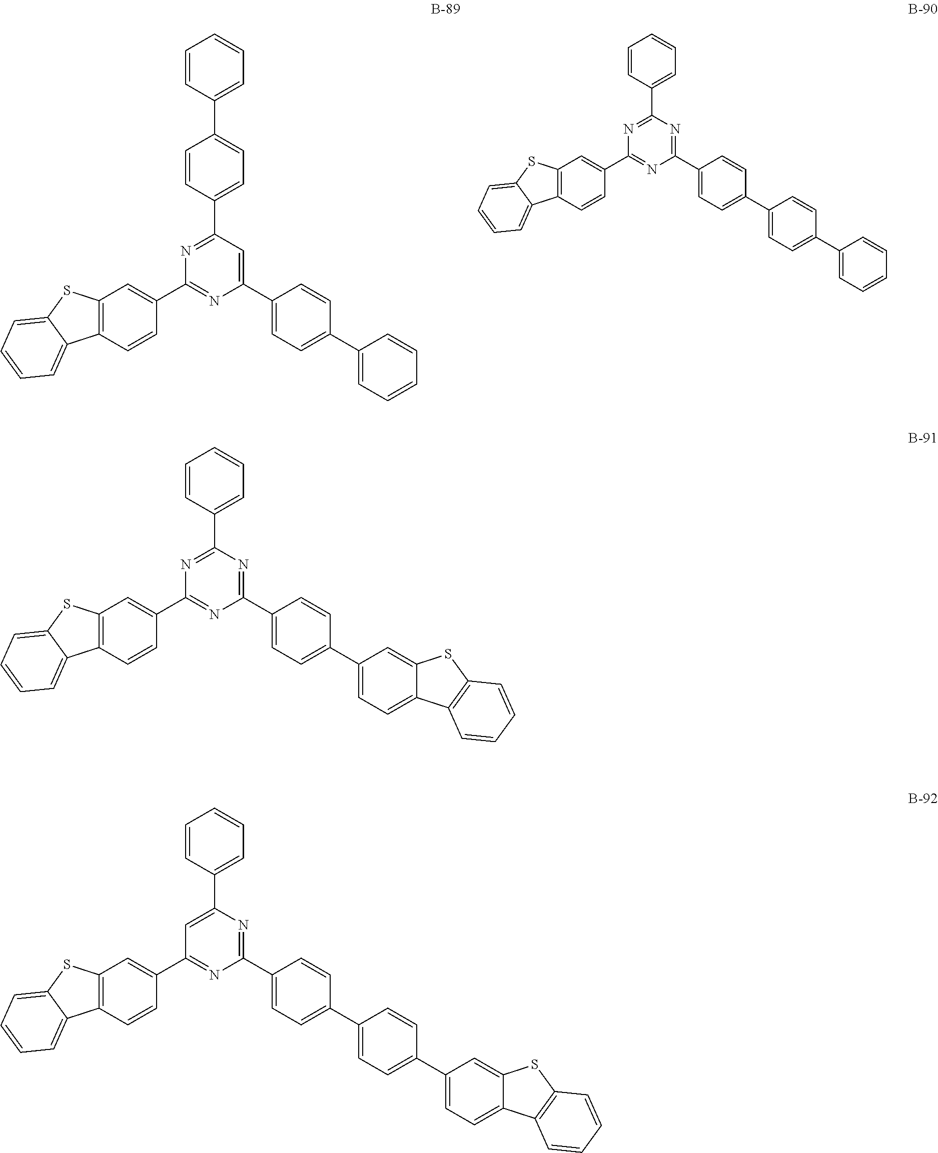 [SMILES: c1ccc(-c2ccc(-c3cc(-c4ccc(-c5ccccc5)cc4)nc(-c4ccc5c(c4)sc4ccccc45)n3)cc2)cc1 ; c1ccc(-c2ccc(-c3ccc(-c4nc(-c5ccccc5)nc(-c5ccc6c(c5)sc5ccccc56)n4)cc3)cc2)cc1 ; c1ccc(-c2nc(-c3ccc(-c4ccc5c(c4)sc4ccccc45)cc3)nc(-c3ccc4c(c3)sc3ccccc34)n2)cc1 ; c1ccc(-c2cc(-c3ccc4c(c3)sc3ccccc34)nc(-c3ccc(-c4ccc(-c5ccc6c(c5)sc5ccccc56)cc4)cc3)n2)cc1]

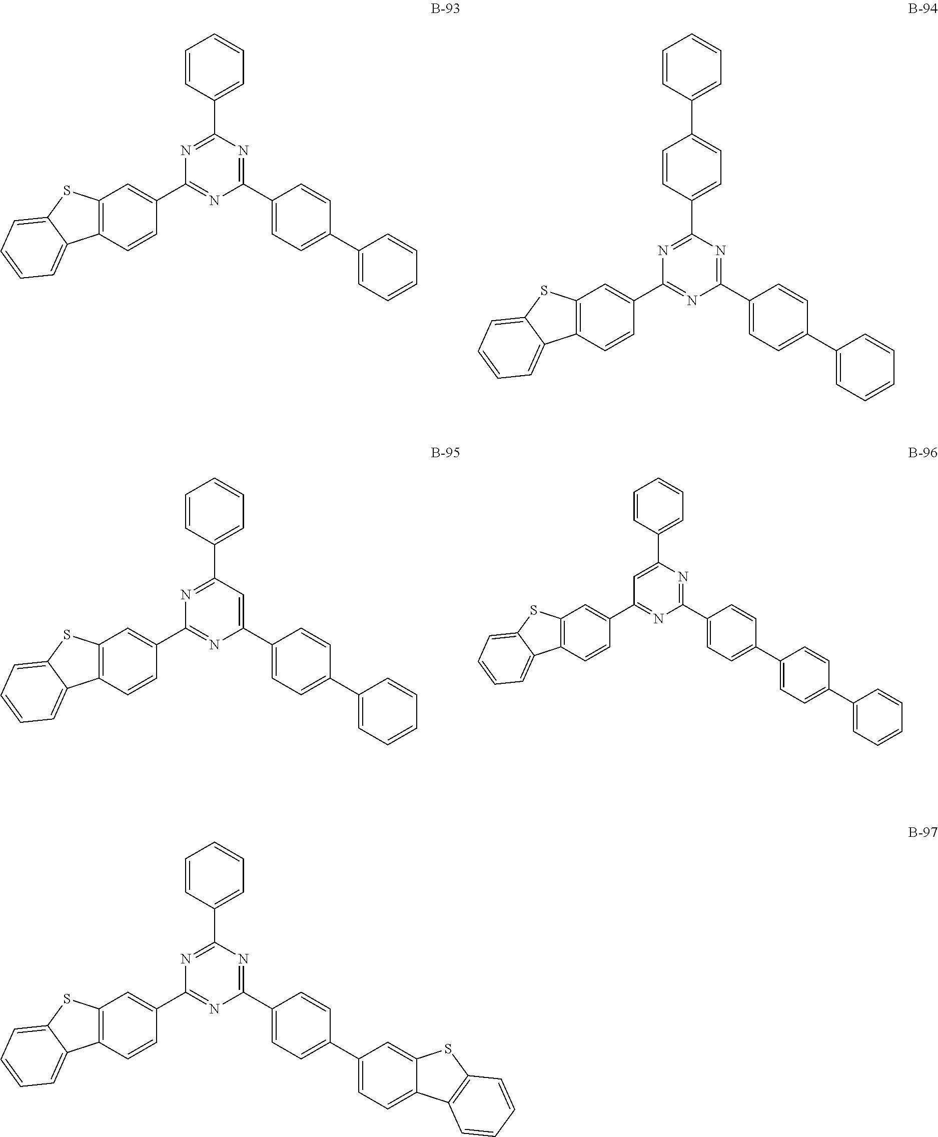 [SMILES: c1ccc(-c2ccc(-c3nc(-c4ccccc4)nc(-c4ccc5c(c4)sc4ccccc45)n3)cc2)cc1 ; c1ccc(-c2ccc(-c3nc(-c4ccc(-c5ccccc5)cc4)nc(-c4ccc5c(c4)sc4ccccc45)n3)cc2)cc1 ; c1ccc(-c2ccc(-c3cc(-c4ccccc4)nc(-c4ccc5c(c4)sc4ccccc45)n3)cc2)cc1 ; c1ccc(-c2ccc(-c3ccc(-c4nc(-c5ccccc5)cc(-c5ccc6c(c5)sc5ccccc56)n4)cc3)cc2)cc1 ; c1ccc(-c2nc(-c3ccc(-c4ccc5c(c4)sc4ccccc45)cc3)nc(-c3ccc4c(c3)sc3ccccc34)n2)cc1]

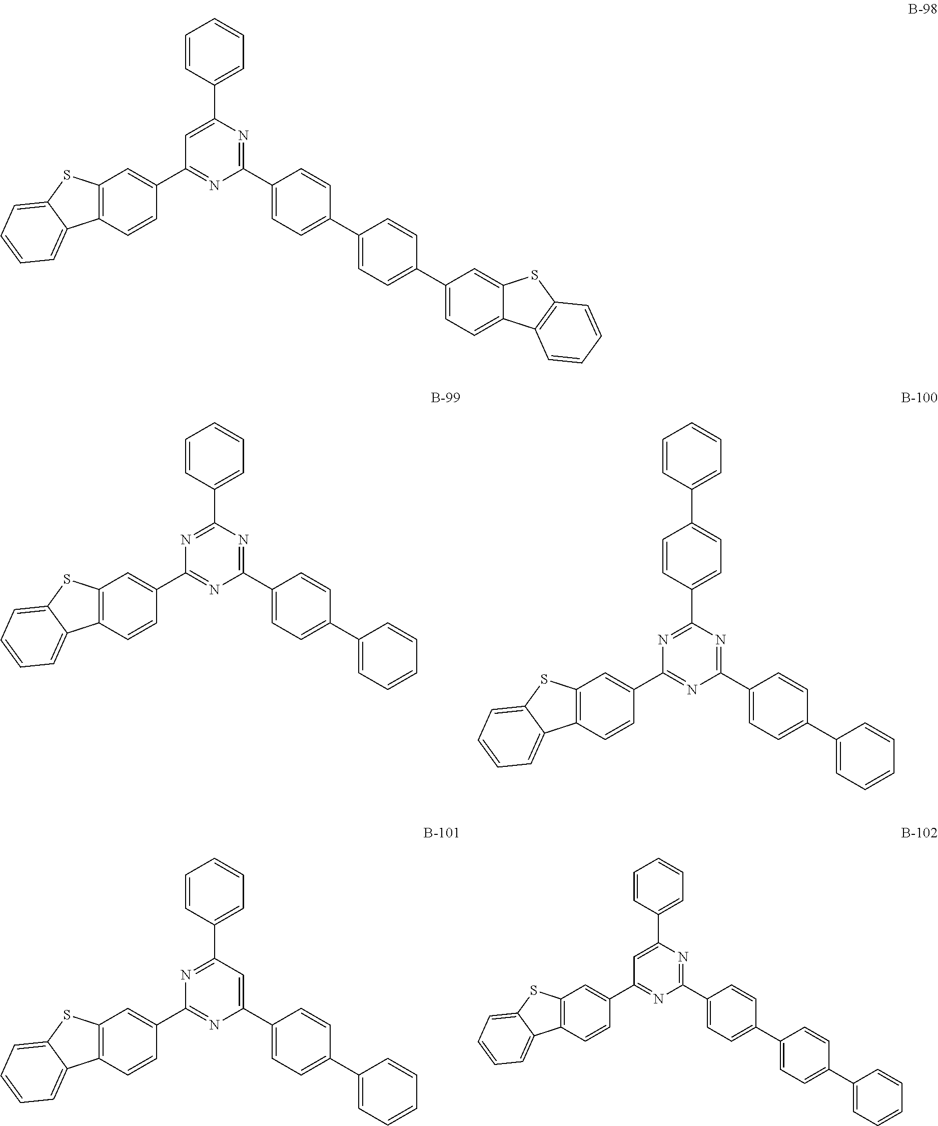 [SMILES: c1ccc(-c2cc(-c3ccc4c(c3)sc3ccccc34)nc(-c3ccc(-c4ccc(-c5ccc6c(c5)sc5ccccc56)cc4)cc3)n2)cc1 ; c1ccc(-c2ccc(-c3nc(-c4ccccc4)nc(-c4ccc5c(c4)sc4ccccc45)n3)cc2)cc1 ; c1ccc(-c2ccc(-c3nc(-c4ccc(-c5ccccc5)cc4)nc(-c4ccc5c(c4)sc4ccccc45)n3)cc2)cc1 ; c1ccc(-c2ccc(-c3cc(-c4ccccc4)nc(-c4ccc5c(c4)sc4ccccc45)n3)cc2)cc1 ; c1ccc(-c2ccc(-c3ccc(-c4nc(-c5ccccc5)cc(-c5ccc6c(c5)sc5ccccc56)n4)cc3)cc2)cc1]

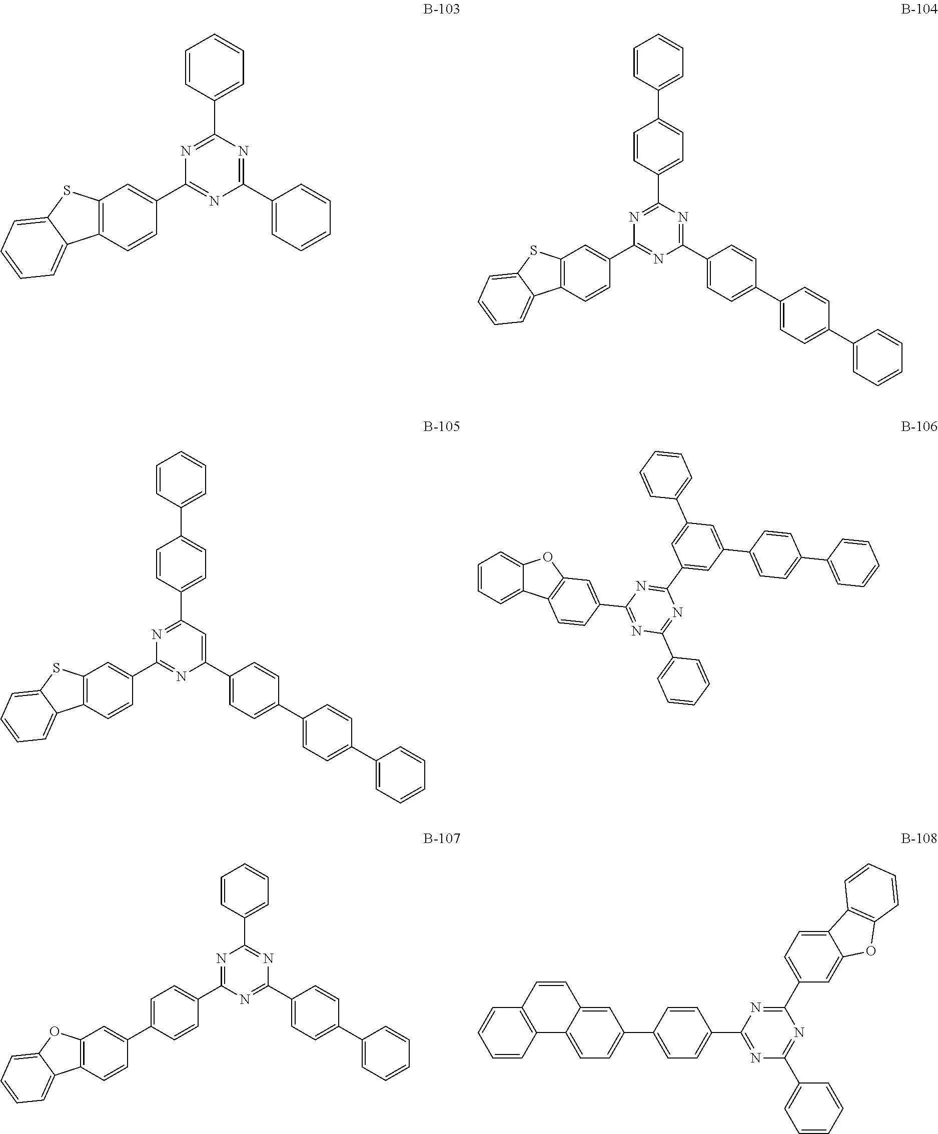 [SMILES: c1ccc(-c2nc(-c3ccccc3)nc(-c3ccc4c(c3)sc3ccccc34)n2)cc1 ; c1ccc(-c2ccc(-c3ccc(-c4nc(-c5ccc(-c6ccccc6)cc5)nc(-c5ccc6c(c5)sc5ccccc56)n4)cc3)cc2)cc1 ; c1ccc(-c2ccc(-c3ccc(-c4cc(-c5ccc(-c6ccccc6)cc5)nc(-c5ccc6c(c5)sc5ccccc56)n4)cc3)cc2)cc1 ; c1ccc(-c2ccc(-c3cc(-c4ccccc4)cc(-c4nc(-c5ccccc5)nc(-c5ccc6c(c5)oc5ccccc56)n4)c3)cc2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2nc(-c3ccc(-c4ccc5c(ccc6ccccc65)c4)cc3)nc(-c3ccc4c(c3)oc3ccccc34)n2)cc1]

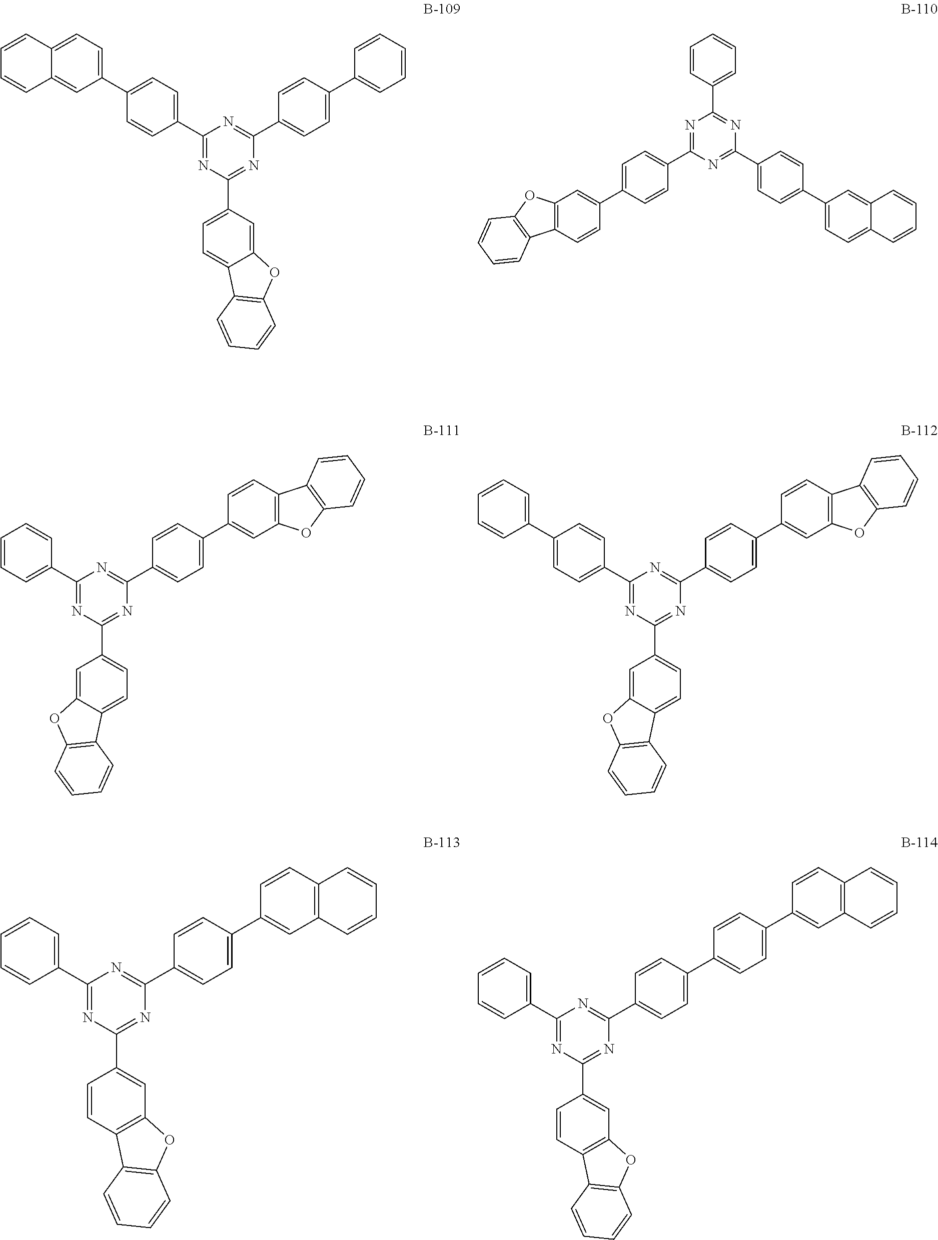 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2nc(-c3ccc(-c4ccc5ccccc5c4)cc3)nc(-c3ccc(-c4ccc5c(c4)oc4ccccc45)cc3)n2)cc1 ; c1ccc(-c2nc(-c3ccc(-c4ccc5c(c4)oc4ccccc45)cc3)nc(-c3ccc4c(c3)oc3ccccc34)n2)cc1 ; c1ccc(-c2ccc(-c3nc(-c4ccc(-c5ccc6c(c5)oc5ccccc56)cc4)nc(-c4ccc5c(c4)oc4ccccc45)n3)cc2)cc1 ; c1ccc(-c2nc(-c3ccc(-c4ccc5ccccc5c4)cc3)nc(-c3ccc4c(c3)oc3ccccc34)n2)cc1 ; c1ccc(-c2nc(-c3ccc(-c4ccc(-c5ccc6ccccc6c5)cc4)cc3)nc(-c3ccc4c(c3)oc3ccccc34)n2)cc1]

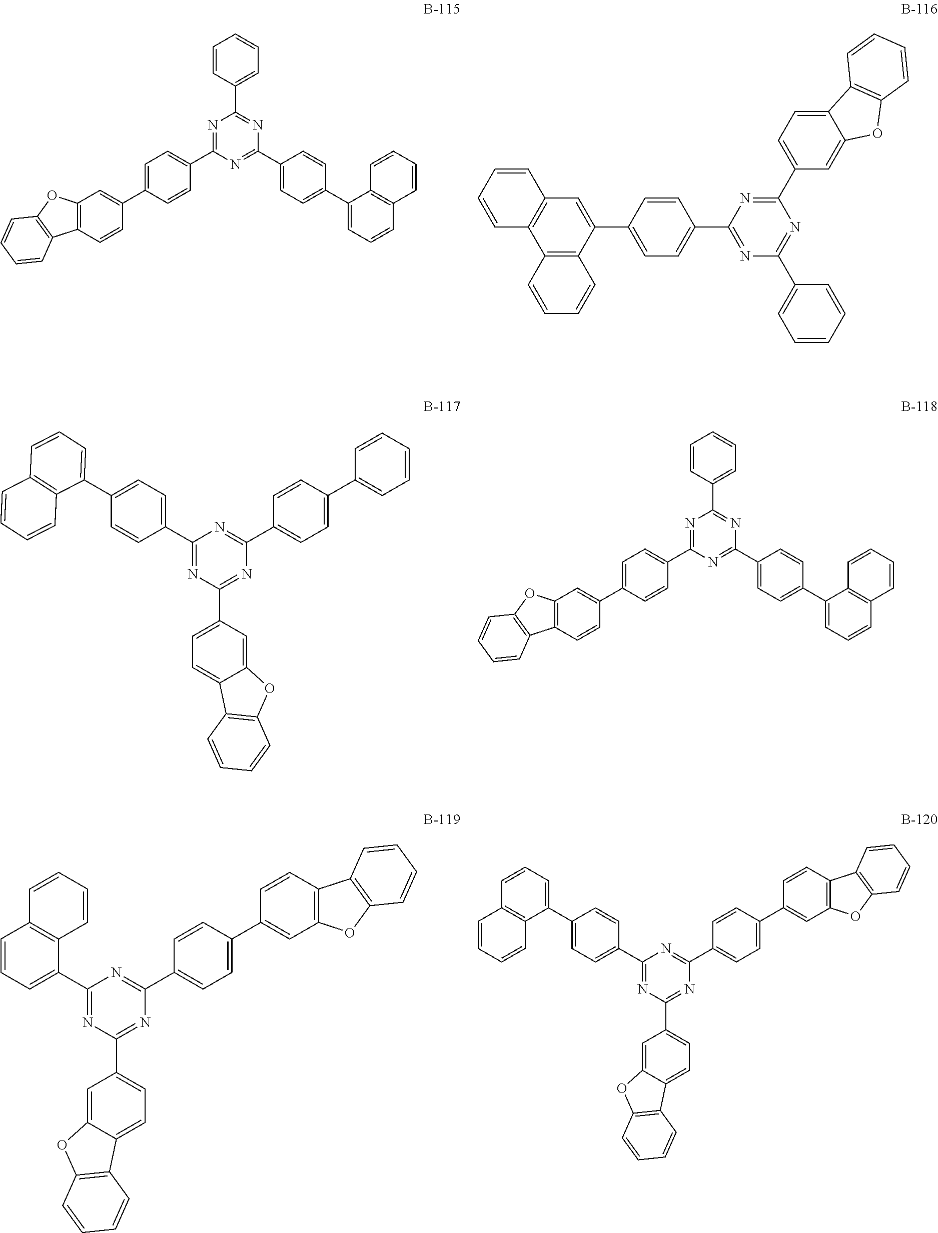

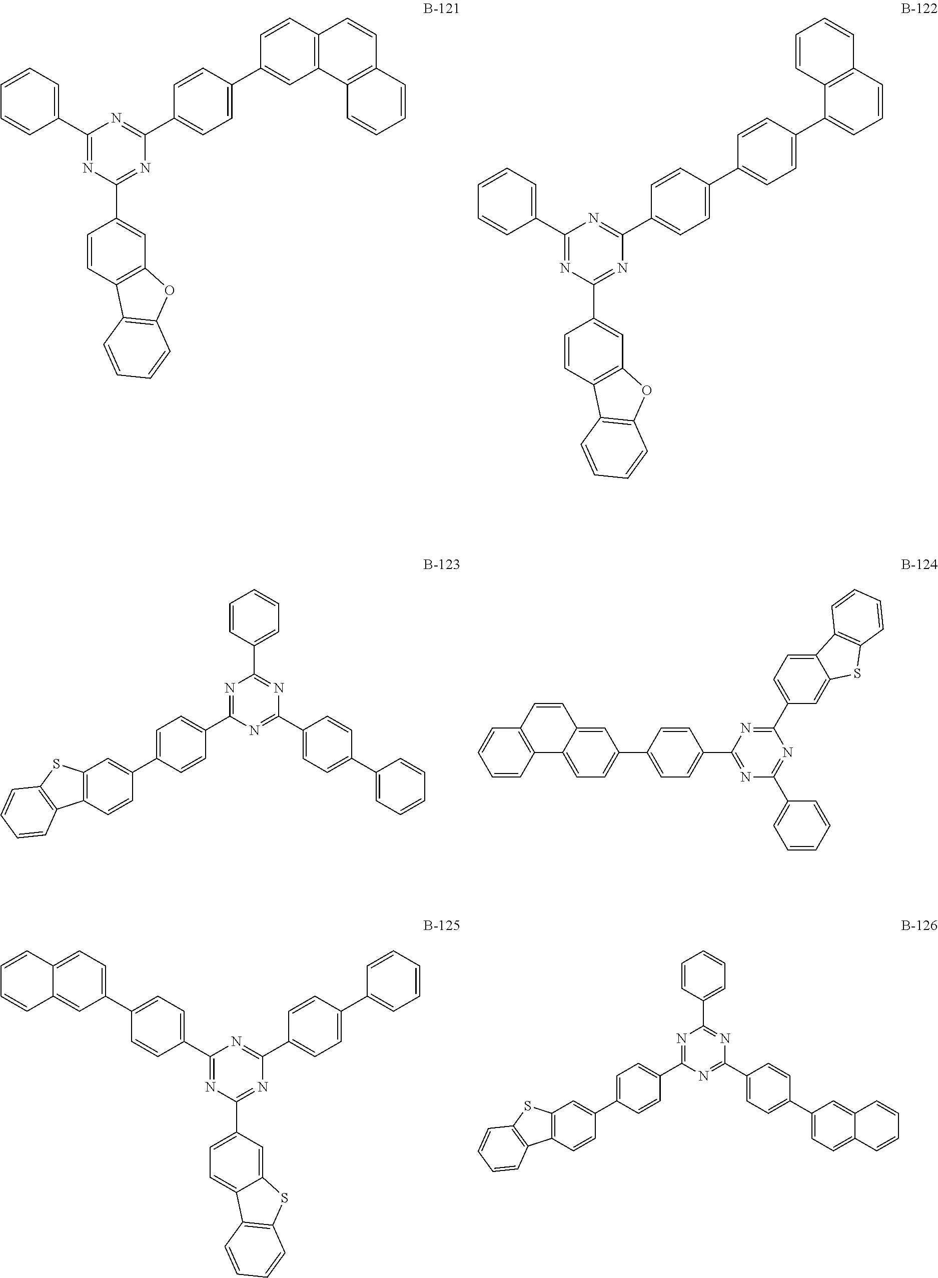 [SMILES: c1ccc(-c2nc(-c3ccc(-c4ccc5ccc6ccccc6c5c4)cc3)nc(-c3ccc4c(c3)oc3ccccc34)n2)cc1 ; c1ccc(-c2nc(-c3ccc(-c4ccc(-c5cccc6ccccc56)cc4)cc3)nc(-c3ccc4c(c3)oc3ccccc34)n2)cc1 ; c1ccc(-c2ccc(-c3nc(-c4ccccc4)nc(-c4ccc(-c5ccc6c(c5)sc5ccccc56)cc4)n3)cc2)cc1 ; c1ccc(-c2nc(-c3ccc(-c4ccc5c(ccc6ccccc65)c4)cc3)nc(-c3ccc4c(c3)sc3ccccc34)n2)cc1 ; c1ccc(-c2ccc(-c3nc(-c4ccc(-c5ccc6ccccc6c5)cc4)nc(-c4ccc5c(c4)sc4ccccc45)n3)cc2)cc1 ; c1ccc(-c2nc(-c3ccc(-c4ccc5ccccc5c4)cc3)nc(-c3ccc(-c4ccc5c(c4)sc4ccccc45)cc3)n2)cc1]

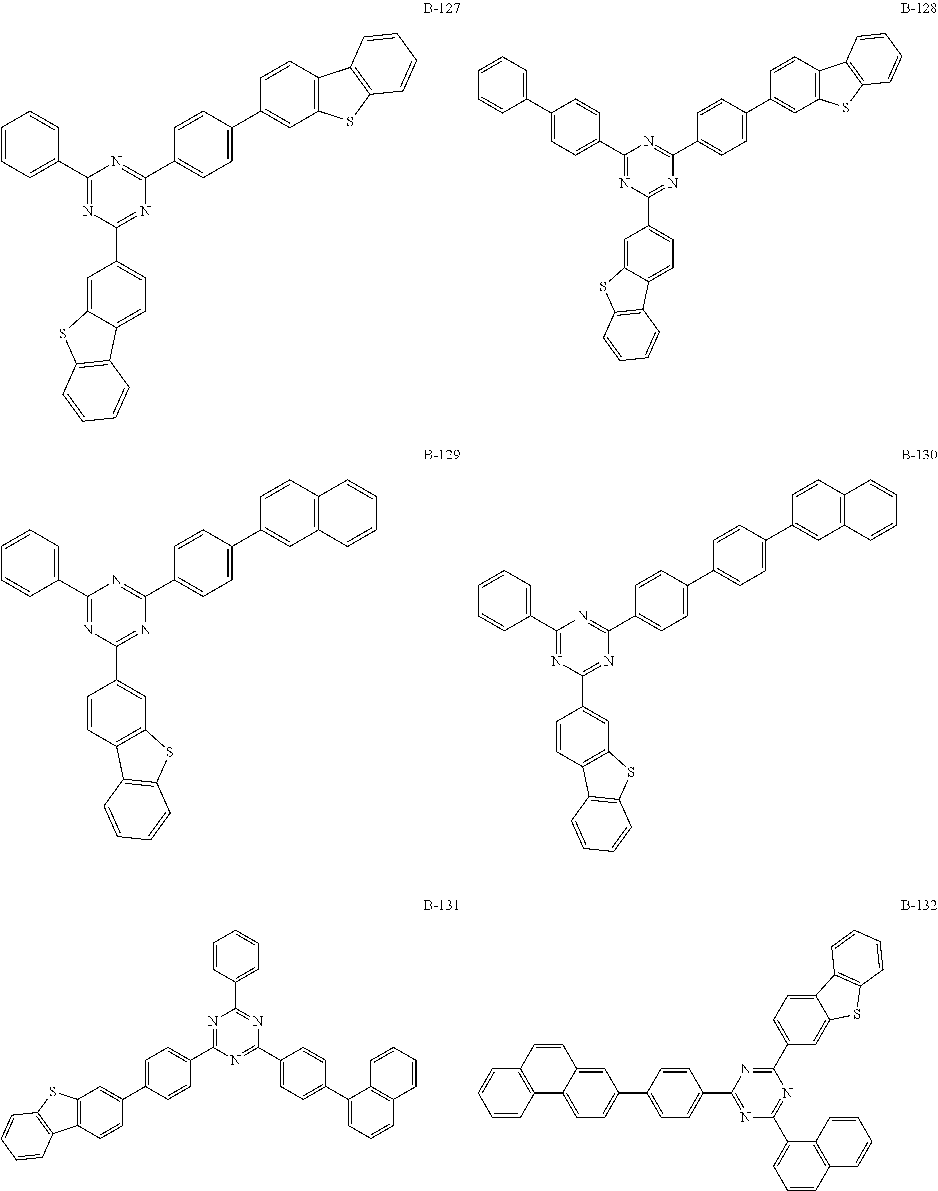

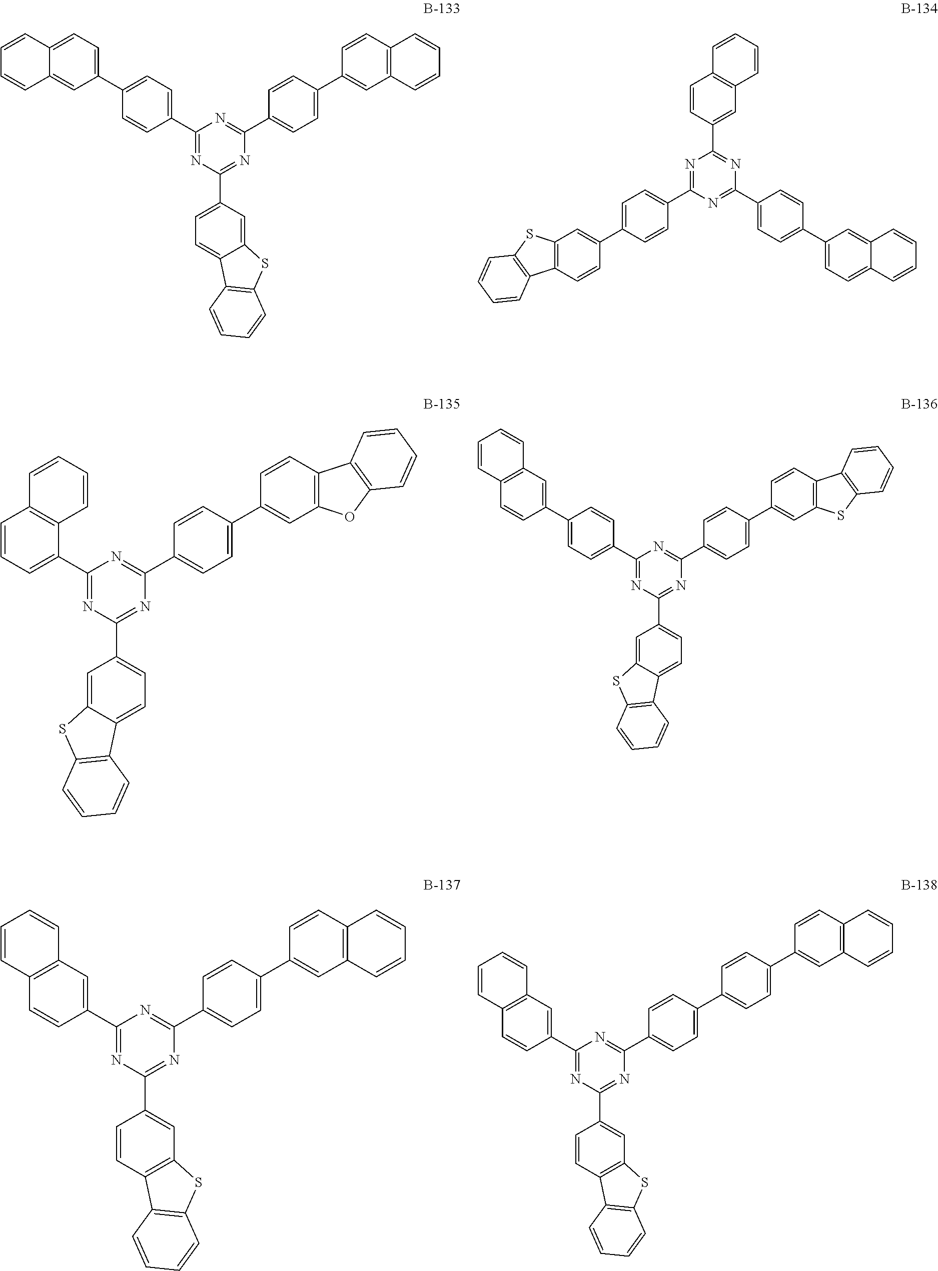

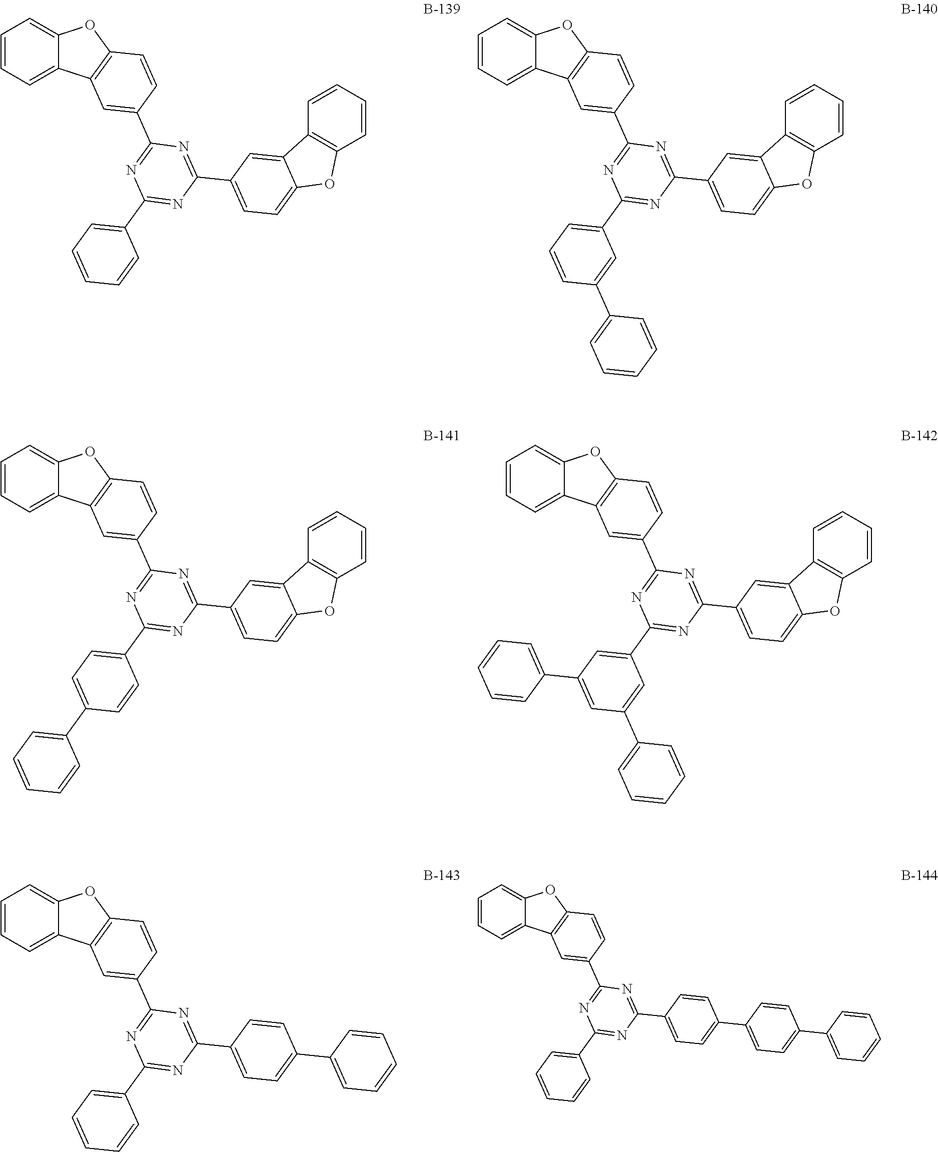 [SMILES: c1ccc(-c2nc(-c3ccc4oc5ccccc5c4c3)nc(-c3ccc4oc5ccccc5c4c3)n2)cc1 ; c1ccc(-c2cccc(-c3nc(-c4ccc5oc6ccccc6c5c4)nc(-c4ccc5oc6ccccc6c5c4)n3)c2)cc1 ; c1ccc(-c2ccc(-c3nc(-c4ccc5oc6ccccc6c5c4)nc(-c4ccc5oc6ccccc6c5c4)n3)cc2)cc1 ; c1ccc(-c2cc(-c3ccccc3)cc(-c3nc(-c4ccc5oc6ccccc6c5c4)nc(-c4ccc5oc6ccccc6c5c4)n3)c2)cc1 ; c1ccc(-c2ccc(-c3nc(-c4ccccc4)nc(-c4ccc5oc6ccccc6c5c4)n3)cc2)cc1 ; c1ccc(-c2ccc(-c3ccc(-c4nc(-c5ccccc5)nc(-c5ccc6oc7ccccc7c6c5)n4)cc3)cc2)cc1]

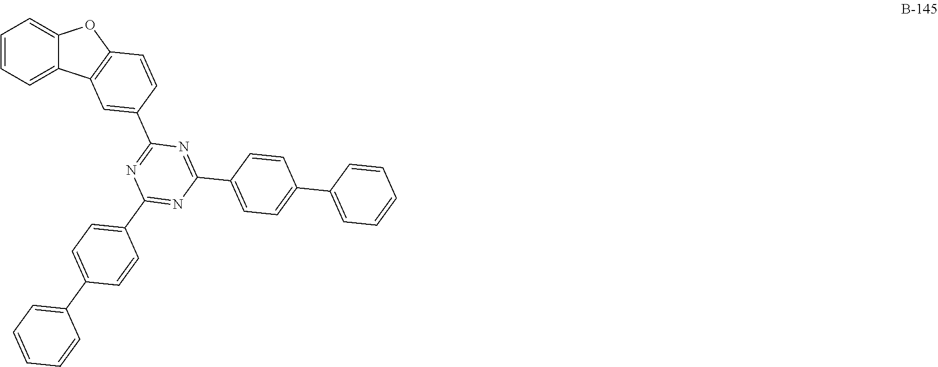 [SMILES: c1ccc(-c2ccc(-c3nc(-c4ccc(-c5ccccc5)cc4)nc(-c4ccc5oc6ccccc6c5c4)n3)cc2)cc1]

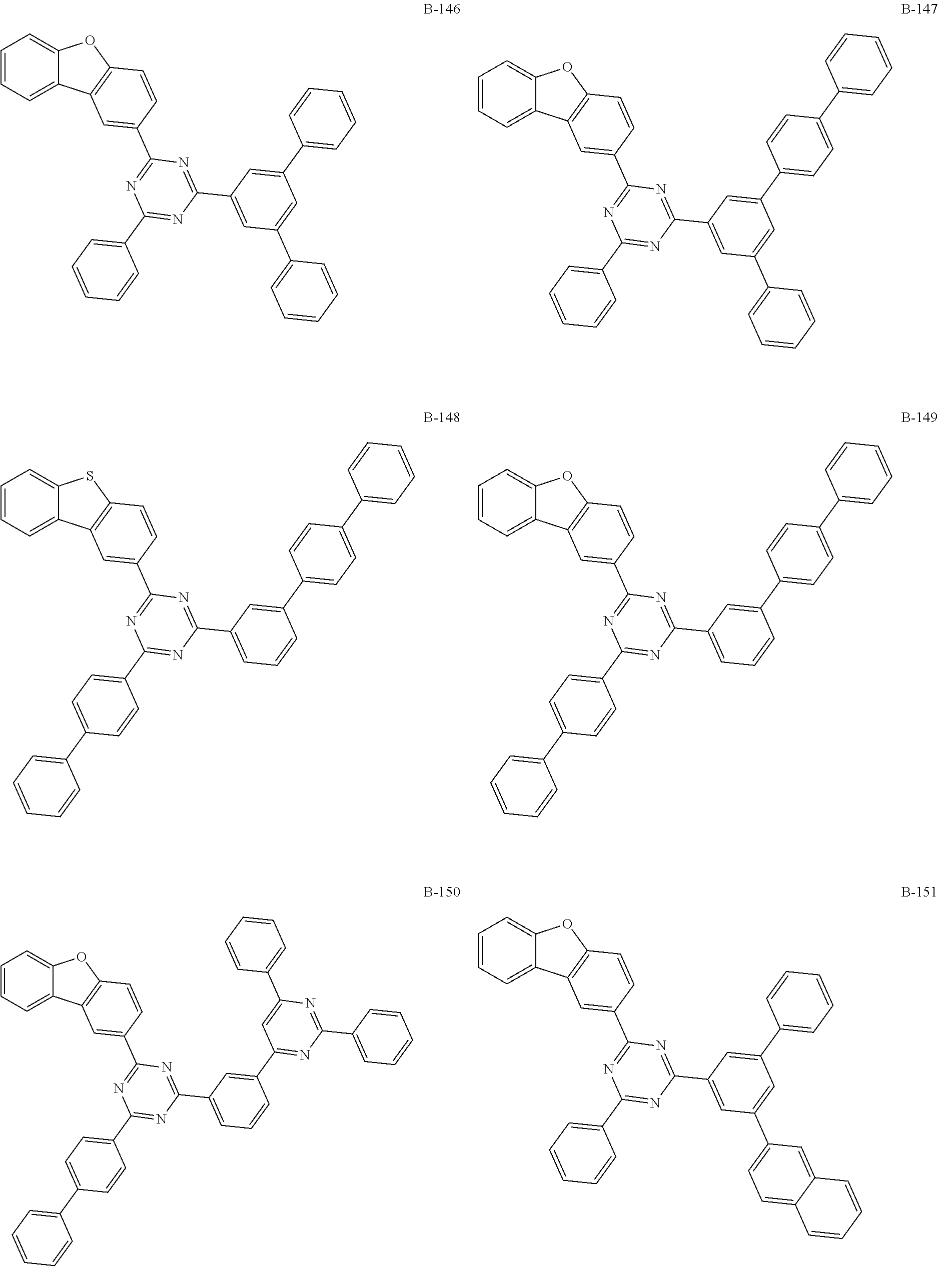

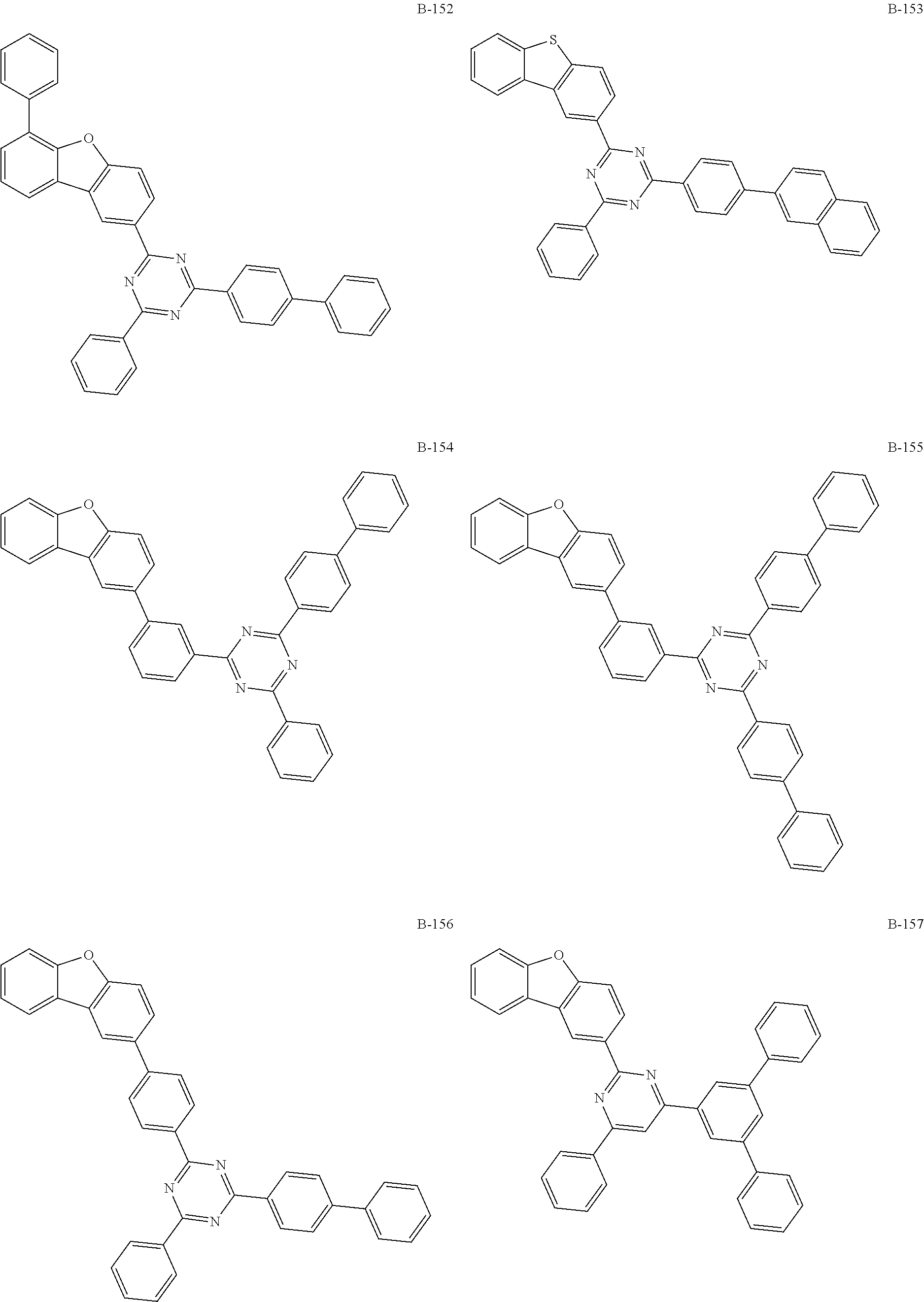 [SMILES: c1ccc(-c2ccc(-c3nc(-c4ccccc4)nc(-c4ccc5oc6c(-c7ccccc7)cccc6c5c4)n3)cc2)cc1 ; c1ccc(-c2nc(-c3ccc(-c4ccc5ccccc5c4)cc3)nc(-c3ccc4sc5ccccc5c4c3)n2)cc1 ; c1ccc(-c2ccc(-c3nc(-c4ccccc4)nc(-c4cccc(-c5ccc6oc7ccccc7c6c5)c4)n3)cc2)cc1 ; c1ccc(-c2ccc(-c3nc(-c4ccc(-c5ccccc5)cc4)nc(-c4cccc(-c5ccc6oc7ccccc7c6c5)c4)n3)cc2)cc1 ; c1ccc(-c2ccc(-c3nc(-c4ccccc4)nc(-c4ccc(-c5ccc6oc7ccccc7c6c5)cc4)n3)cc2)cc1 ; c1ccc(-c2cc(-c3ccccc3)cc(-c3cc(-c4ccccc4)nc(-c4ccc5oc6ccccc6c5c4)n3)c2)cc1]

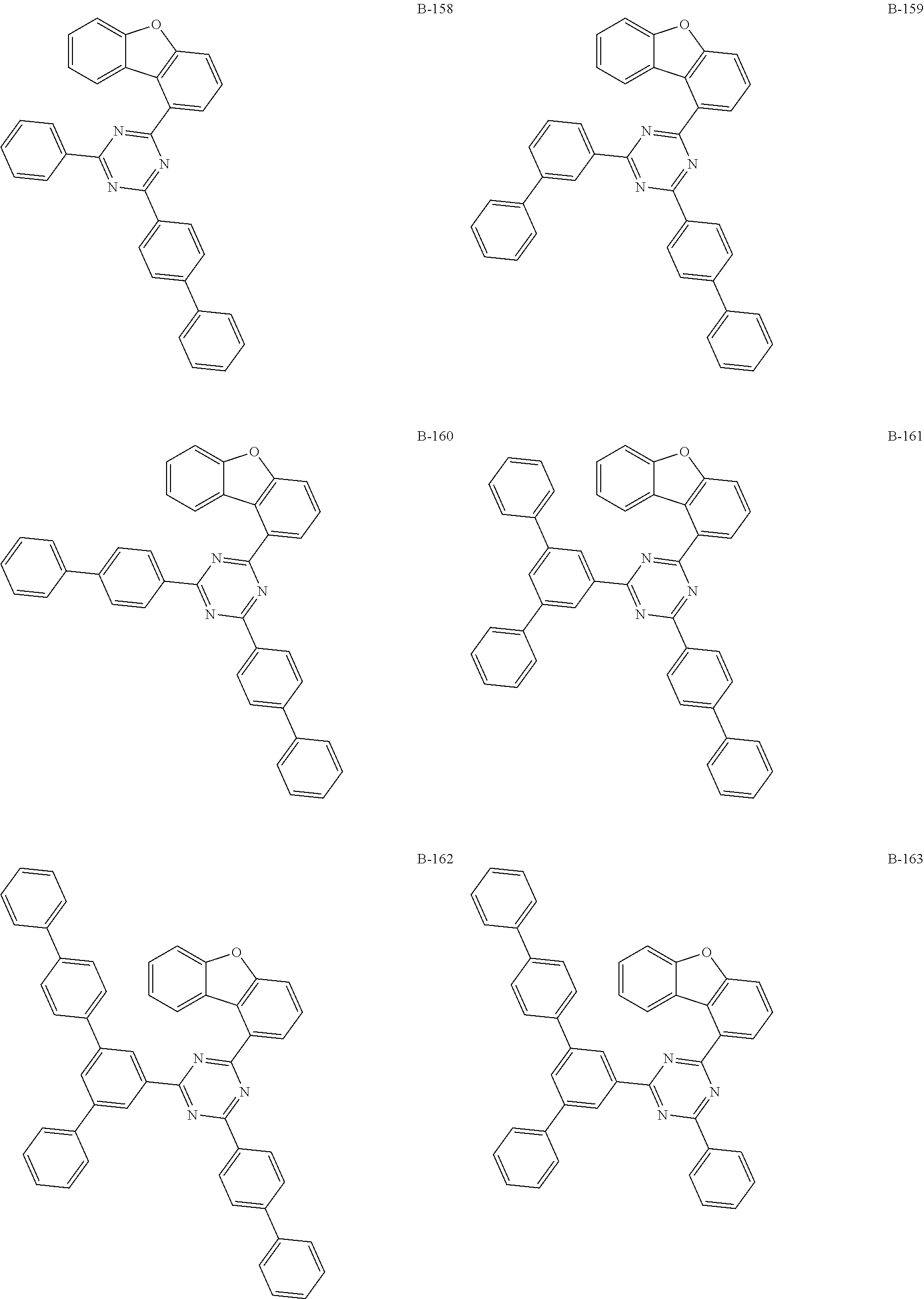 [SMILES: c1ccc(-c2ccc(-c3nc(-c4ccccc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cc(-c4ccccc4)cc(-c4nc(-c5ccc(-c6ccccc6)cc5)nc(-c5cccc6oc7ccccc7c56)n4)c3)cc2)cc1 ; c1ccc(-c2ccc(-c3cc(-c4ccccc4)cc(-c4nc(-c5ccccc5)nc(-c5cccc6oc7ccccc7c56)n4)c3)cc2)cc1]

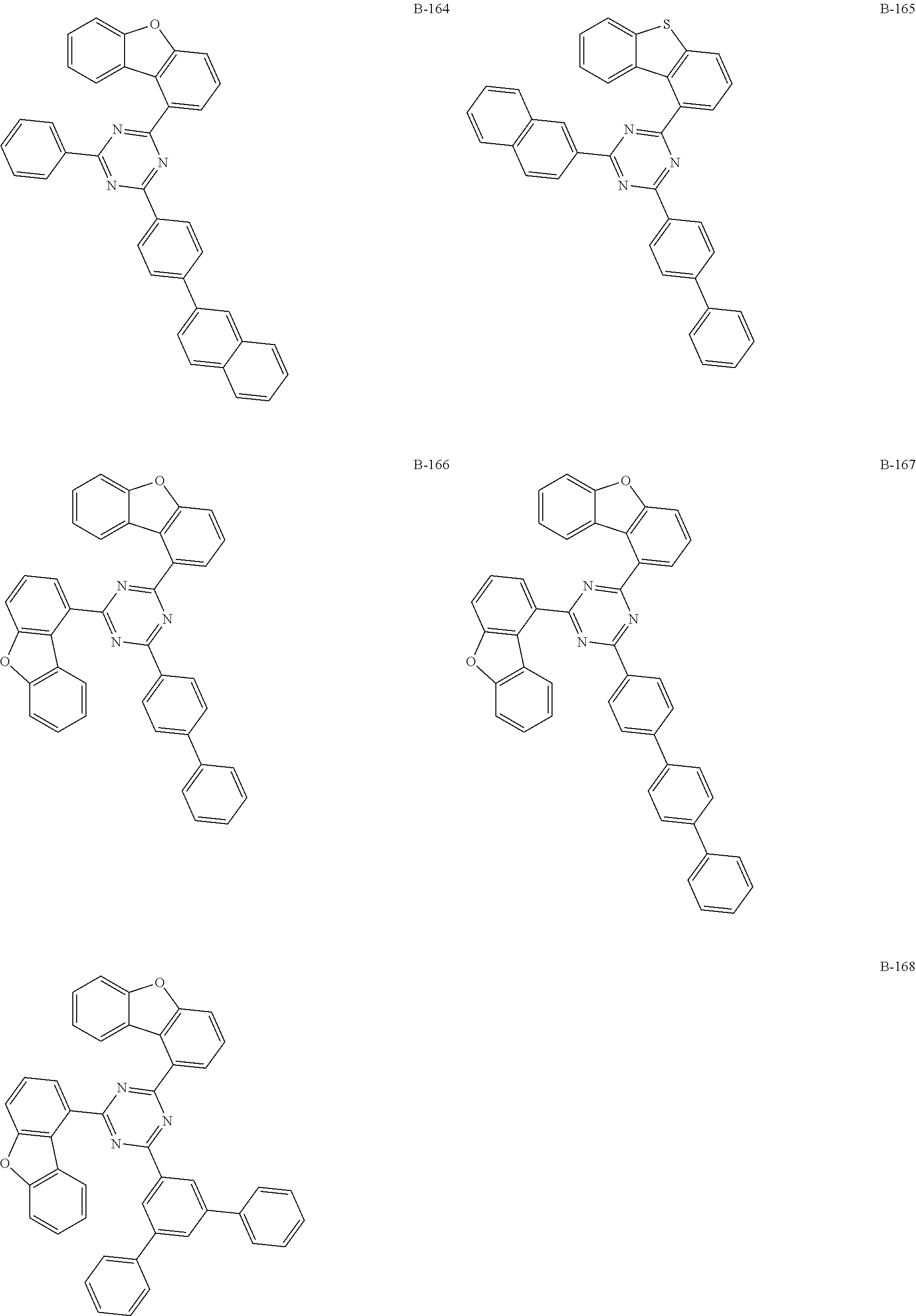 [SMILES: c1ccc(-c2nc(-c3ccc(-c4ccc5ccccc5c4)cc3)nc(-c3cccc4oc5ccccc5c34)n2)cc1 ; c1ccc(-c2ccc(-c3nc(-c4ccc5ccccc5c4)nc(-c4cccc5sc6ccccc6c45)n3)cc2)cc1 ; c1ccc(-c2ccc(-c3nc(-c4cccc5oc6ccccc6c45)nc(-c4cccc5oc6ccccc6c45)n3)cc2)cc1 ; c1ccc(-c2ccc(-c3ccc(-c4nc(-c5cccc6oc7ccccc7c56)nc(-c5cccc6oc7ccccc7c56)n4)cc3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1]

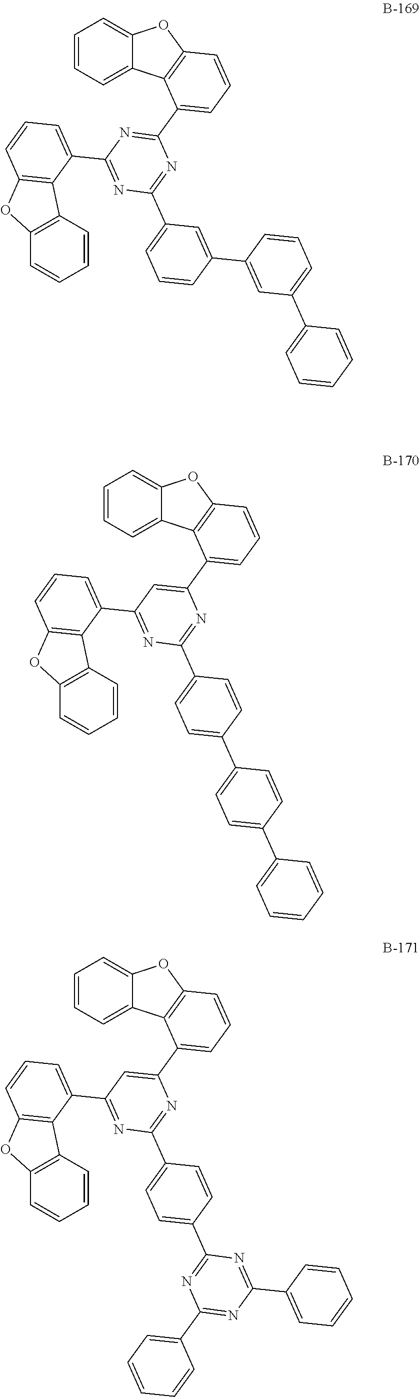 [SMILES: c1ccc(-c2cccc(-c3cccc(-c4nc(-c5cccc6oc7ccccc7c56)nc(-c5cccc6oc7ccccc7c56)n4)c3)c2)cc1 ; c1ccc(-c2ccc(-c3ccc(-c4nc(-c5cccc6oc7ccccc7c56)cc(-c5cccc6oc7ccccc7c56)n4)cc3)cc2)cc1 ; c1ccc(-c2nc(-c3ccccc3)nc(-c3ccc(-c4nc(-c5cccc6oc7ccccc7c56)cc(-c5cccc6oc7ccccc7c56)n4)cc3)n2)cc1]

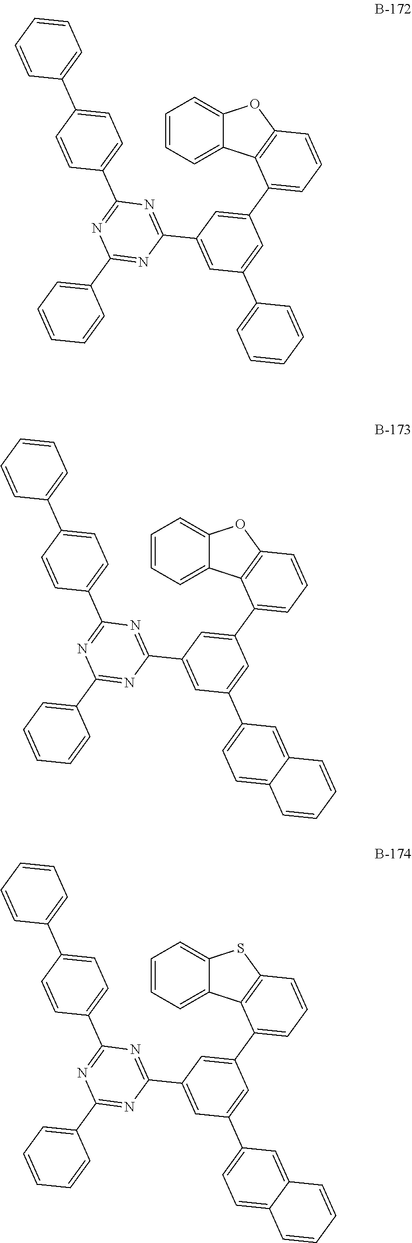 [SMILES: c1ccc(-c2ccc(-c3nc(-c4ccccc4)nc(-c4cc(-c5ccccc5)cc(-c5cccc6oc7ccccc7c56)c4)n3)cc2)cc1 ; c1ccc(-c2ccc(-c3nc(-c4ccccc4)nc(-c4cc(-c5ccc6ccccc6c5)cc(-c5cccc6oc7ccccc7c56)c4)n3)cc2)cc1 ; c1ccc(-c2ccc(-c3nc(-c4ccccc4)nc(-c4cc(-c5ccc6ccccc6c5)cc(-c5cccc6sc7ccccc7c56)c4)n3)cc2)cc1]

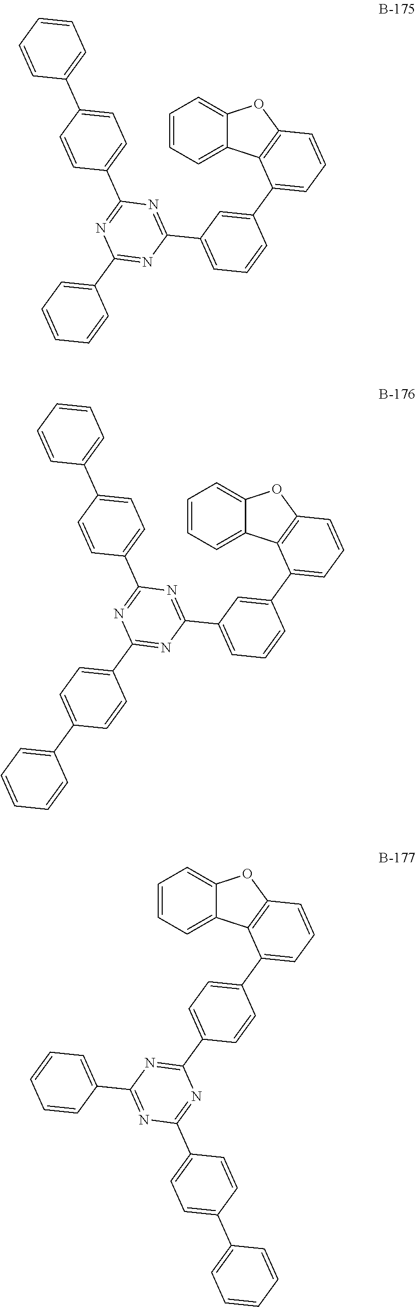 [SMILES: c1ccc(-c2ccc(-c3nc(-c4ccccc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4ccccc4)nc(-c4ccc(-c5cccc6oc7ccccc7c56)cc4)n3)cc2)cc1]

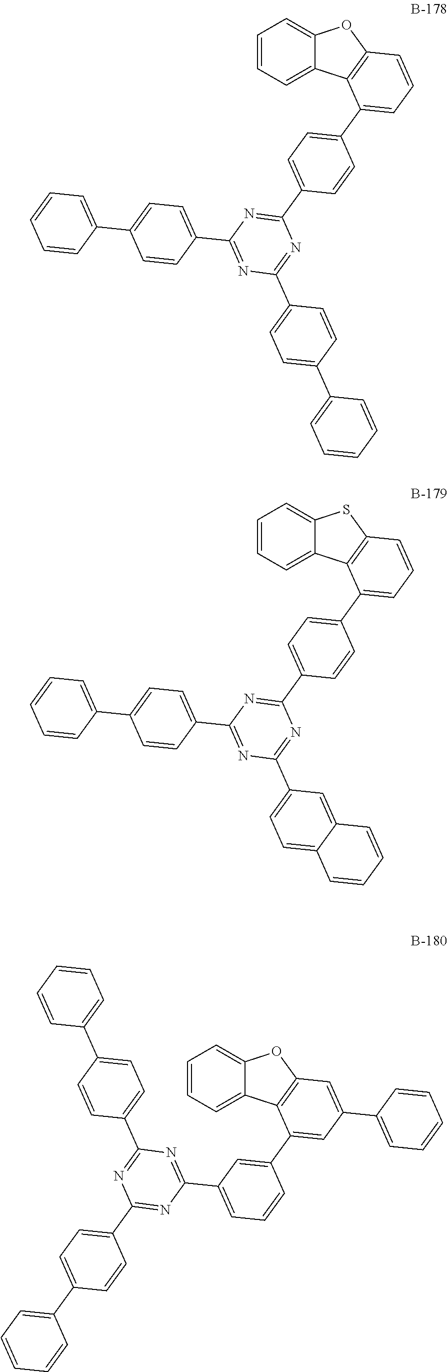 [SMILES: c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5cccc6sc7ccccc7c56)cc4)nc(-c4ccc5ccccc5c4)n3)cc2)cc1 ; c1ccc(-c2ccc(-c3nc(-c4ccc(-c5ccccc5)cc4)nc(-c4cccc(-c5cc(-c6ccccc6)cc6oc7ccccc7c56)c4)n3)cc2)cc1]

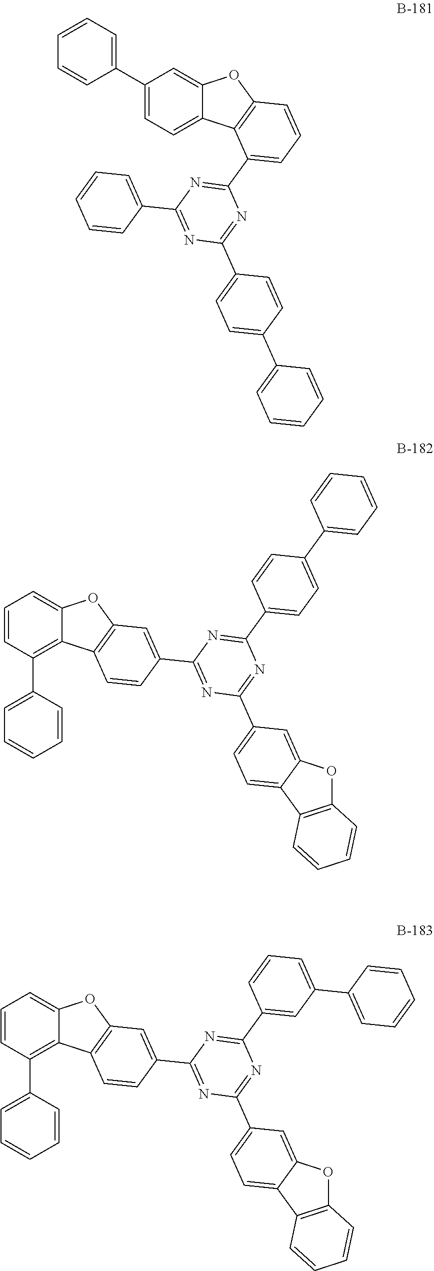 [SMILES: c1ccc(-c2ccc(-c3nc(-c4ccccc4)nc(-c4cccc5oc6cc(-c7ccccc7)ccc6c45)n3)cc2)cc1 ; c1ccc(-c2ccc(-c3nc(-c4ccc5c(c4)oc4ccccc45)nc(-c4ccc5c(c4)oc4cccc(-c6ccccc6)c45)n3)cc2)cc1 ; c1ccc(-c2cccc(-c3nc(-c4ccc5c(c4)oc4ccccc45)nc(-c4ccc5c(c4)oc4cccc(-c6ccccc6)c45)n3)c2)cc1]

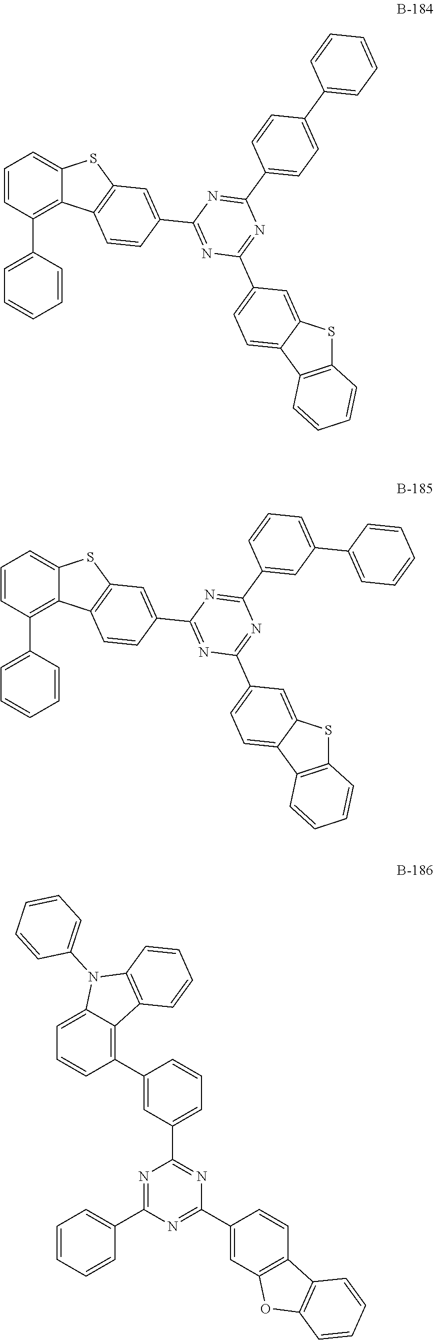 [SMILES: c1ccc(-c2ccc(-c3nc(-c4ccc5c(c4)sc4ccccc45)nc(-c4ccc5c(c4)sc4cccc(-c6ccccc6)c45)n3)cc2)cc1 ; c1ccc(-c2cccc(-c3nc(-c4ccc5c(c4)sc4ccccc45)nc(-c4ccc5c(c4)sc4cccc(-c6ccccc6)c45)n3)c2)cc1 ; c1ccc(-c2nc(-c3cccc(-c4cccc5c4c4ccccc4n5-c4ccccc4)c3)nc(-c3ccc4c(c3)oc3ccccc34)n2)cc1]

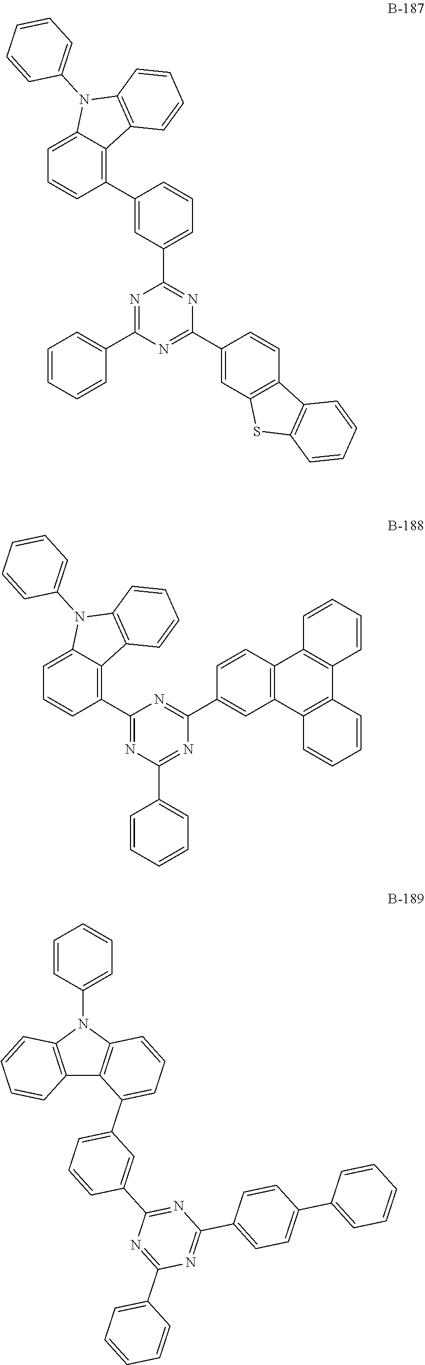 [SMILES: c1ccc(-c2nc(-c3cccc(-c4cccc5c4c4ccccc4n5-c4ccccc4)c3)nc(-c3ccc4c(c3)sc3ccccc34)n2)cc1 ; c1ccc(-c2nc(-c3ccc4c5ccccc5c5ccccc5c4c3)nc(-c3cccc4c3c3ccccc3n4-c3ccccc3)n2)cc1 ; c1ccc(-c2ccc(-c3nc(-c4ccccc4)nc(-c4cccc(-c5cccc6c5c5ccccc5n6-c5ccccc5)c4)n3)cc2)cc1]

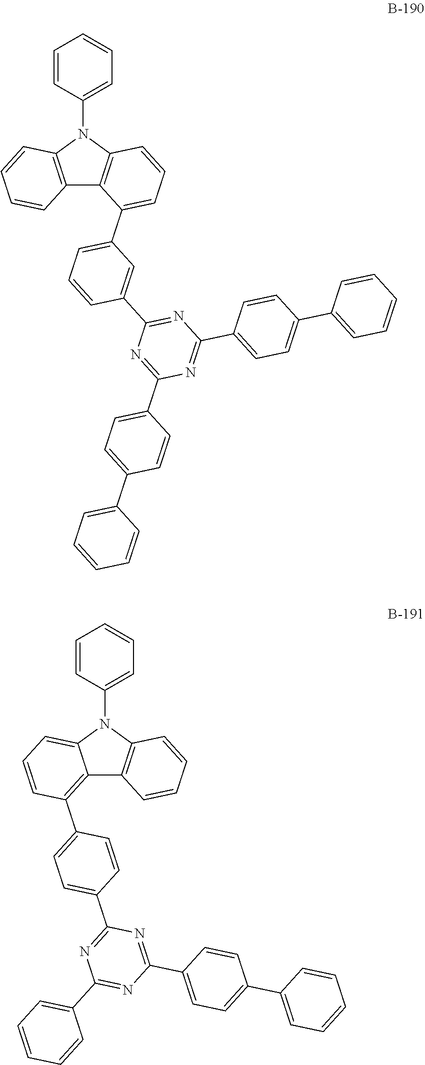 [SMILES: c1ccc(-c2ccc(-c3nc(-c4ccc(-c5ccccc5)cc4)nc(-c4cccc(-c5cccc6c5c5ccccc5n6-c5ccccc5)c4)n3)cc2)cc1 ; c1ccc(-c2ccc(-c3nc(-c4ccccc4)nc(-c4ccc(-c5cccc6c5c5ccccc5n6-c5ccccc5)cc4)n3)cc2)cc1]

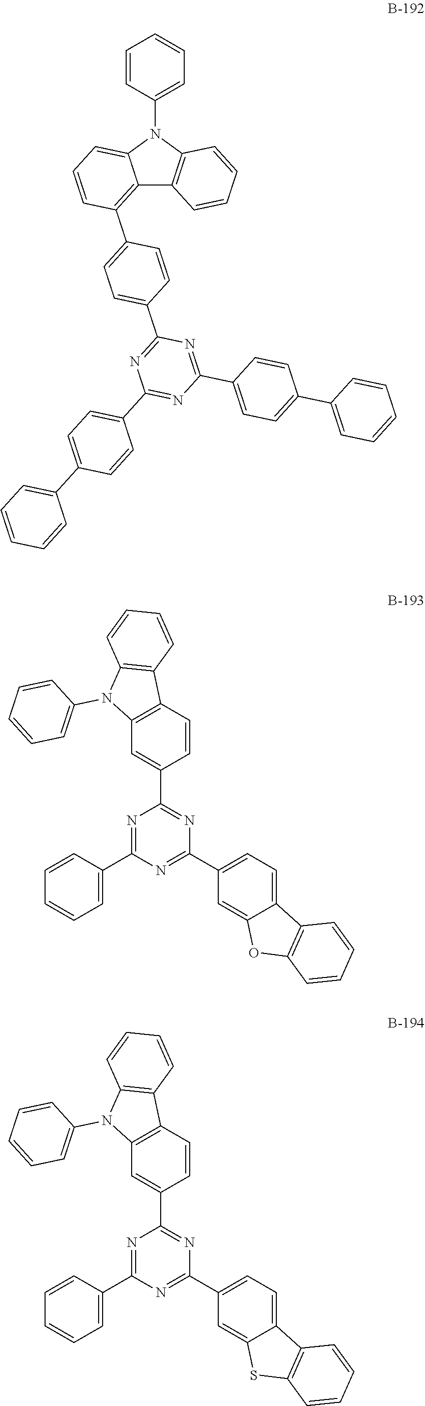 [SMILES: c1ccc(-c2ccc(-c3nc(-c4ccc(-c5ccccc5)cc4)nc(-c4ccc(-c5cccc6c5c5ccccc5n6-c5ccccc5)cc4)n3)cc2)cc1 ; c1ccc(-c2nc(-c3ccc4c(c3)oc3ccccc34)nc(-c3ccc4c5ccccc5n(-c5ccccc5)c4c3)n2)cc1 ; c1ccc(-c2nc(-c3ccc4c(c3)sc3ccccc34)nc(-c3ccc4c5ccccc5n(-c5ccccc5)c4c3)n2)cc1]

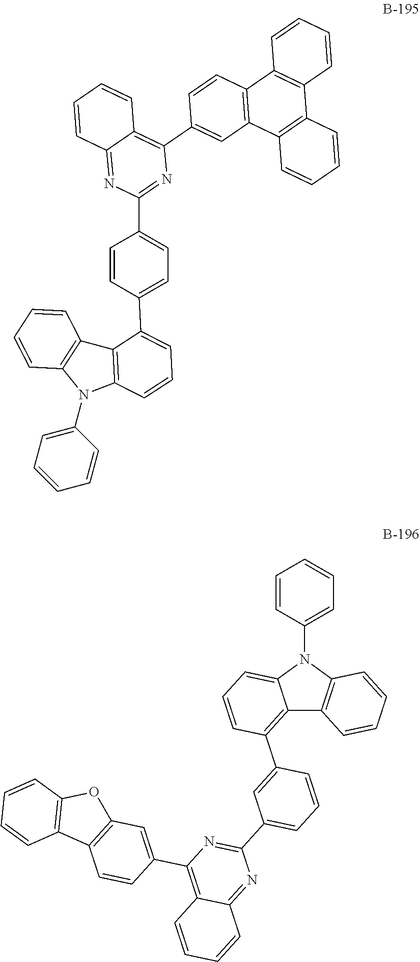 [SMILES: c1ccc(-n2c3ccccc3c3c(-c4ccc(-c5nc(-c6ccc7c8ccccc8c8ccccc8c7c6)c6ccccc6n5)cc4)cccc32)cc1 ; c1ccc(-n2c3ccccc3c3c(-c4cccc(-c5nc(-c6ccc7c(c6)oc6ccccc67)c6ccccc6n5)c4)cccc32)cc1]

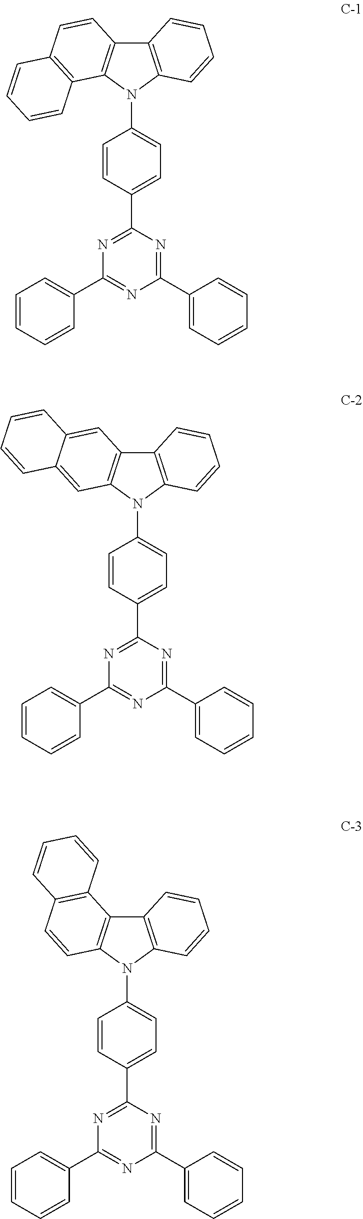 [SMILES: c1ccc(-c2nc(-c3ccccc3)nc(-c3ccc(-n4c5ccccc5c5ccc6ccccc6c54)cc3)n2)cc1 ; c1ccc(-c2nc(-c3ccccc3)nc(-c3ccc(-n4c5ccccc5c5cc6ccccc6cc54)cc3)n2)cc1 ; c1ccc(-c2nc(-c3ccccc3)nc(-c3ccc(-n4c5ccccc5c5c6ccccc6ccc54)cc3)n2)cc1]

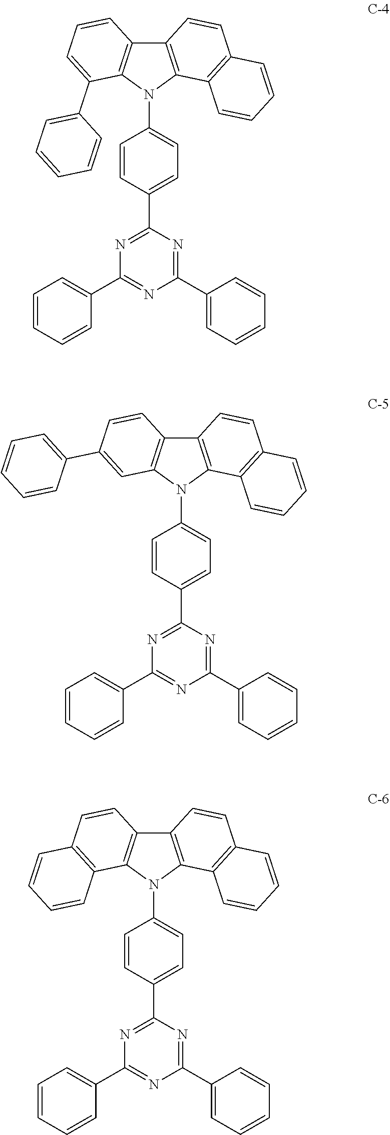 [SMILES: c1ccc(-c2nc(-c3ccccc3)nc(-c3ccc(-n4c5c(-c6ccccc6)cccc5c5ccc6ccccc6c54)cc3)n2)cc1 ; c1ccc(-c2ccc3c4ccc5ccccc5c4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5c6ccccc6ccc5c5ccc6ccccc6c54)cc3)n2)cc1]

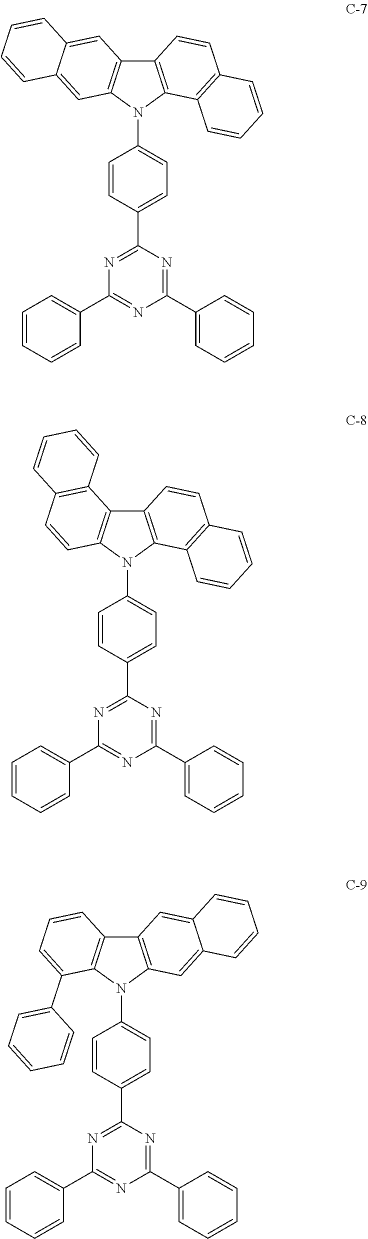 [SMILES: c1ccc(-c2nc(-c3ccccc3)nc(-c3ccc(-n4c5cc6ccccc6cc5c5ccc6ccccc6c54)cc3)n2)cc1 ; c1ccc(-c2nc(-c3ccccc3)nc(-c3ccc(-n4c5ccc6ccccc6c5c5ccc6ccccc6c54)cc3)n2)cc1 ; c1ccc(-c2nc(-c3ccccc3)nc(-c3ccc(-n4c5cc6ccccc6cc5c5cccc(-c6ccccc6)c54)cc3)n2)cc1]

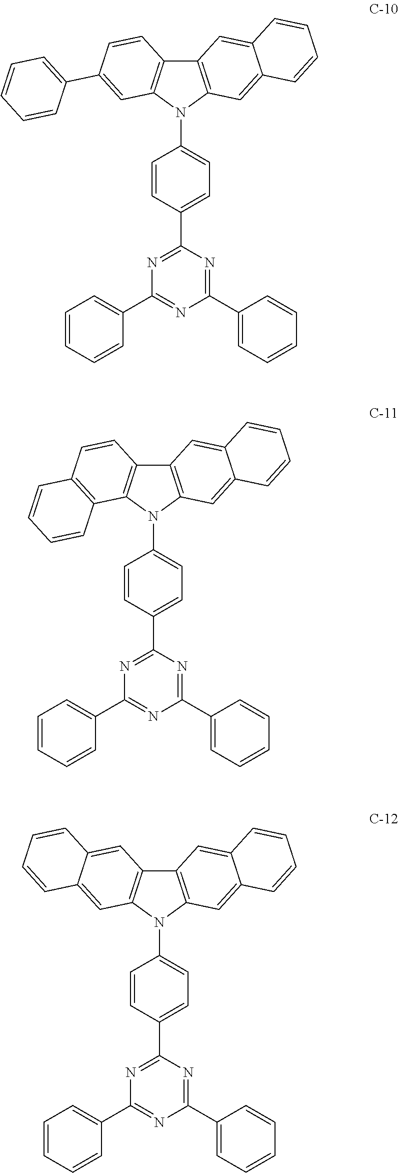 [SMILES: c1ccc(-c2ccc3c4cc5ccccc5cc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5cc6ccccc6cc5c5ccc6ccccc6c54)cc3)n2)cc1 ; c1ccc(-c2nc(-c3ccccc3)nc(-c3ccc(-n4c5cc6ccccc6cc5c5cc6ccccc6cc54)cc3)n2)cc1]

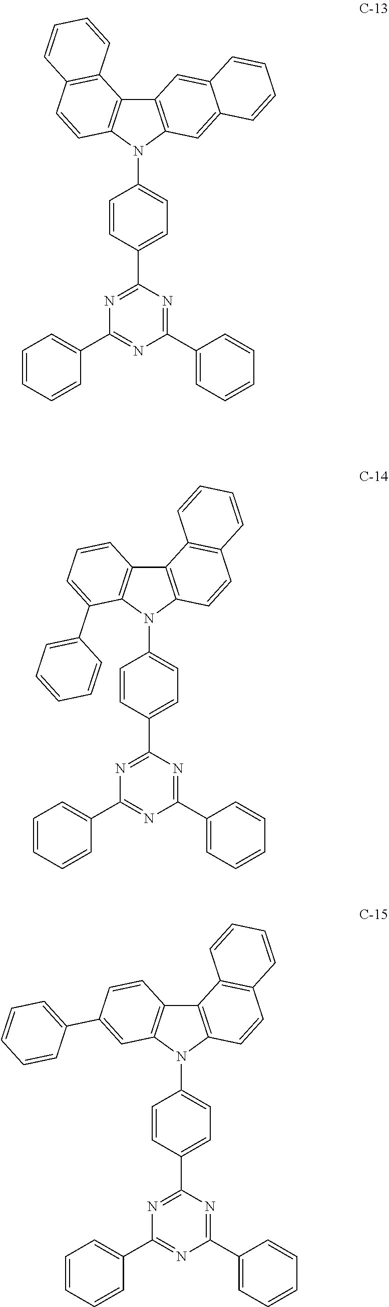 [SMILES: c1ccc(-c2nc(-c3ccccc3)nc(-c3ccc(-n4c5cc6ccccc6cc5c5c6ccccc6ccc54)cc3)n2)cc1 ; c1ccc(-c2nc(-c3ccccc3)nc(-c3ccc(-n4c5ccc6ccccc6c5c5cccc(-c6ccccc6)c54)cc3)n2)cc1 ; c1ccc(-c2ccc3c4c5ccccc5ccc4n(-c4ccc(-c5nc(-c6ccccc6)nc(-c6ccccc6)n5)cc4)c3c2)cc1]

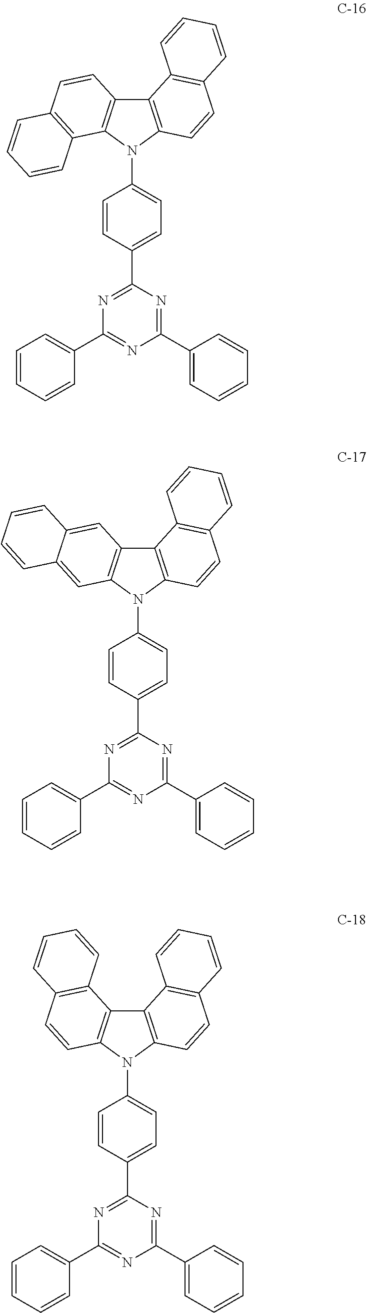 [SMILES: c1ccc(-c2nc(-c3ccccc3)nc(-c3ccc(-n4c5ccc6ccccc6c5c5ccc6ccccc6c54)cc3)n2)cc1 ; c1ccc(-c2nc(-c3ccccc3)nc(-c3ccc(-n4c5cc6ccccc6cc5c5c6ccccc6ccc54)cc3)n2)cc1 ; c1ccc(-c2nc(-c3ccccc3)nc(-c3ccc(-n4c5ccc6ccccc6c5c5c6ccccc6ccc54)cc3)n2)cc1]

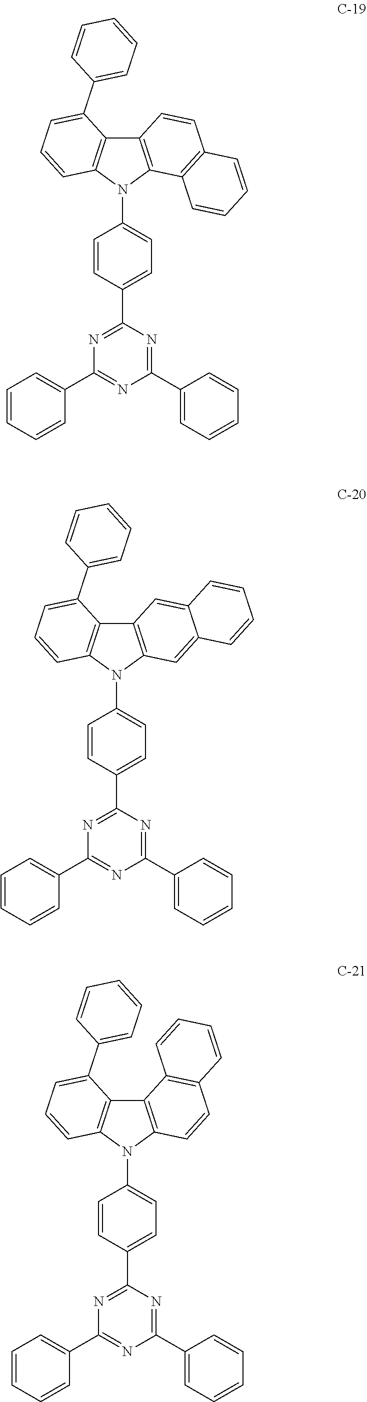 [SMILES: c1ccc(-c2nc(-c3ccccc3)nc(-c3ccc(-n4c5cccc(-c6ccccc6)c5c5ccc6ccccc6c54)cc3)n2)cc1 ; c1ccc(-c2nc(-c3ccccc3)nc(-c3ccc(-n4c5cc6ccccc6cc5c5c(-c6ccccc6)cccc54)cc3)n2)cc1 ; c1ccc(-c2nc(-c3ccccc3)nc(-c3ccc(-n4c5cccc(-c6ccccc6)c5c5c6ccccc6ccc54)cc3)n2)cc1]

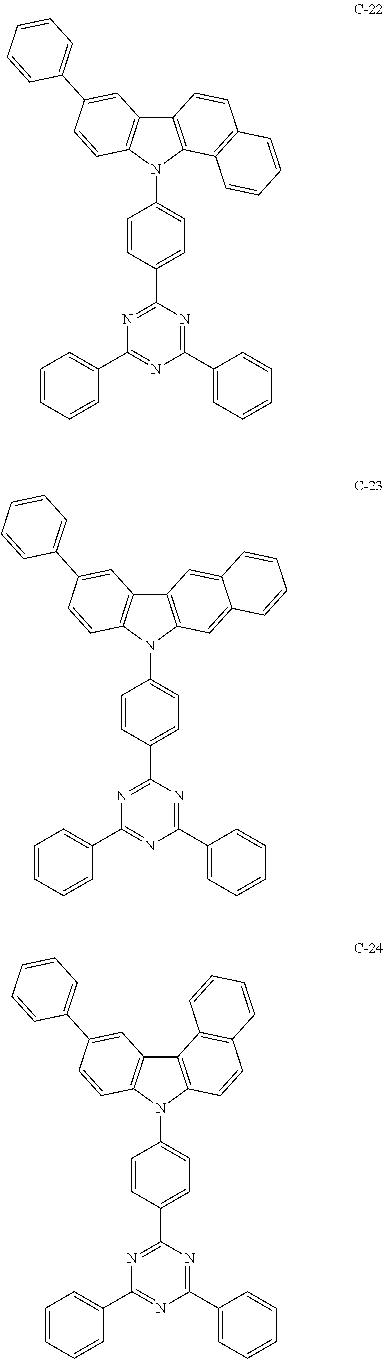 [SMILES: c1ccc(-c2ccc3c(c2)c2ccc4ccccc4c2n3-c2ccc(-c3nc(-c4ccccc4)nc(-c4ccccc4)n3)cc2)cc1 ; c1ccc(-c2ccc3c(c2)c2cc4ccccc4cc2n3-c2ccc(-c3nc(-c4ccccc4)nc(-c4ccccc4)n3)cc2)cc1 ; c1ccc(-c2ccc3c(c2)c2c4ccccc4ccc2n3-c2ccc(-c3nc(-c4ccccc4)nc(-c4ccccc4)n3)cc2)cc1]

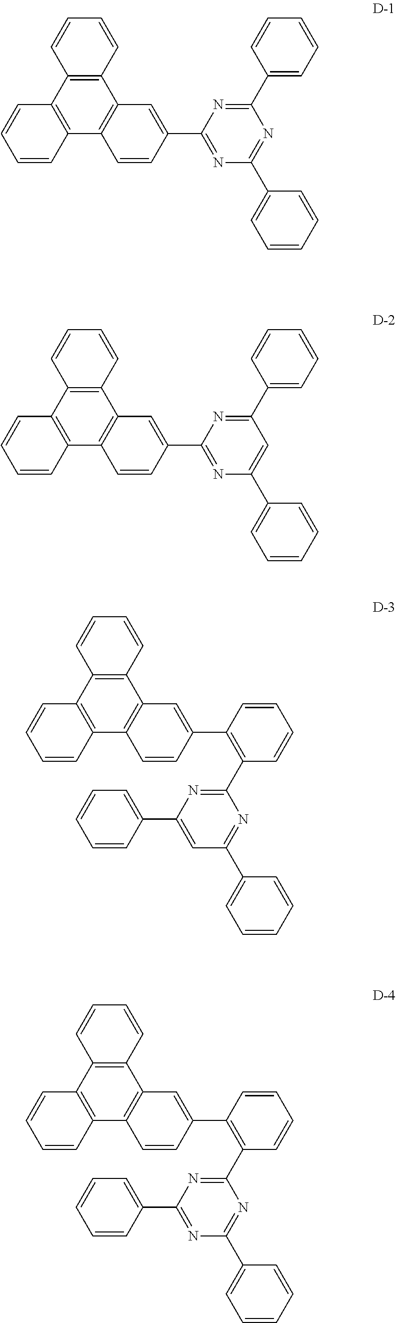 [SMILES: c1ccc(-c2nc(-c3ccccc3)nc(-c3ccc4c5ccccc5c5ccccc5c4c3)n2)cc1 ; c1ccc(-c2cc(-c3ccccc3)nc(-c3ccc4c5ccccc5c5ccccc5c4c3)n2)cc1 ; c1ccc(-c2cc(-c3ccccc3)nc(-c3ccccc3-c3ccc4c5ccccc5c5ccccc5c4c3)n2)cc1 ; c1ccc(-c2nc(-c3ccccc3)nc(-c3ccccc3-c3ccc4c5ccccc5c5ccccc5c4c3)n2)cc1]

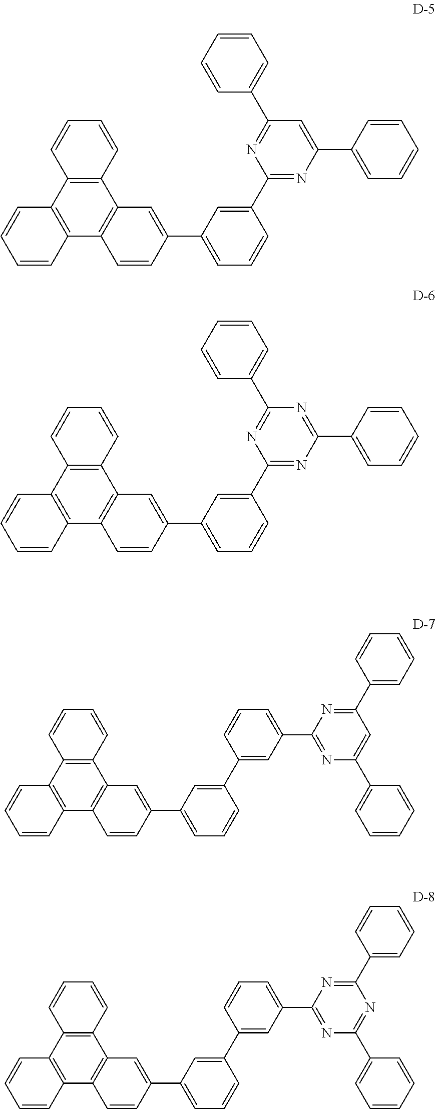 [SMILES: c1ccc(-c2cc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4ccc5c6ccccc6c6ccccc6c5c4)c3)n2)cc1 ; c1ccc(-c2cc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1]

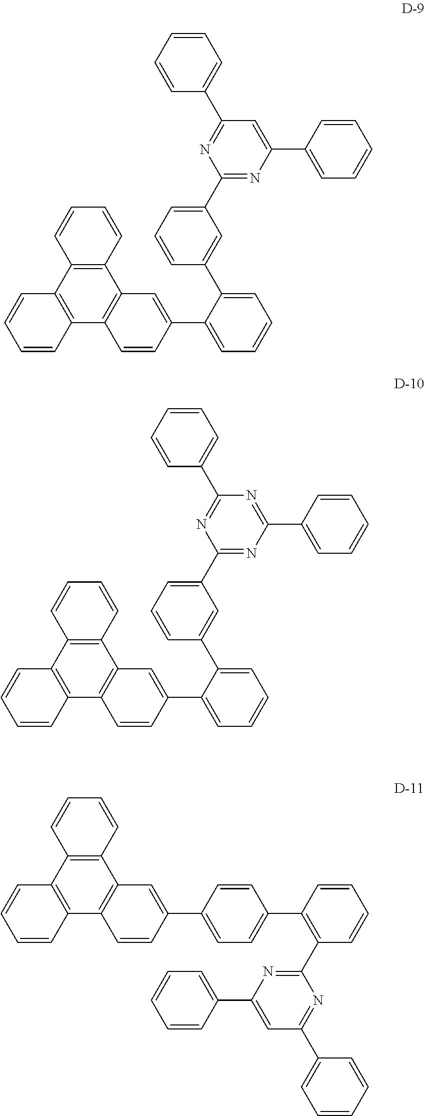 [SMILES: c1ccc(-c2cc(-c3ccccc3)nc(-c3cccc(-c4ccccc4-c4ccc5c6ccccc6c6ccccc6c5c4)c3)n2)cc1 ; c1ccc(-c2nc(-c3ccccc3)nc(-c3cccc(-c4ccccc4-c4ccc5c6ccccc6c6ccccc6c5c4)c3)n2)cc1 ; c1ccc(-c2cc(-c3ccccc3)nc(-c3ccccc3-c3ccc(-c4ccc5c6ccccc6c6ccccc6c5c4)cc3)n2)cc1]

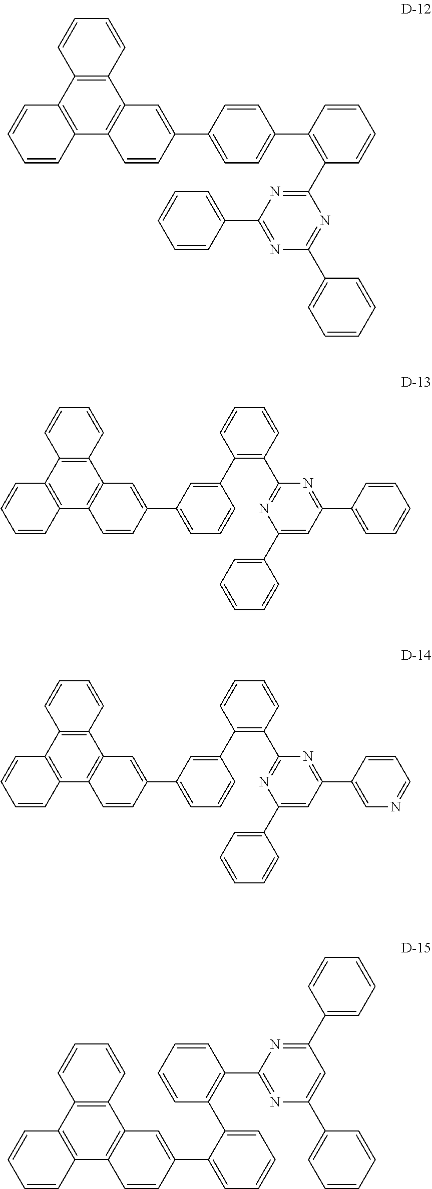 [SMILES: c1ccc(-c2nc(-c3ccccc3)nc(-c3ccccc3-c3ccc(-c4ccc5c6ccccc6c6ccccc6c5c4)cc3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2cc(-c3cccnc3)nc(-c3ccccc3-c3cccc(-c4ccc5c6ccccc6c6ccccc6c5c4)c3)n2)cc1 ; c1ccc(-c2cc(-c3ccccc3)nc(-c3ccccc3-c3ccccc3-c3ccc4c5ccccc5c5ccccc5c4c3)n2)cc1]

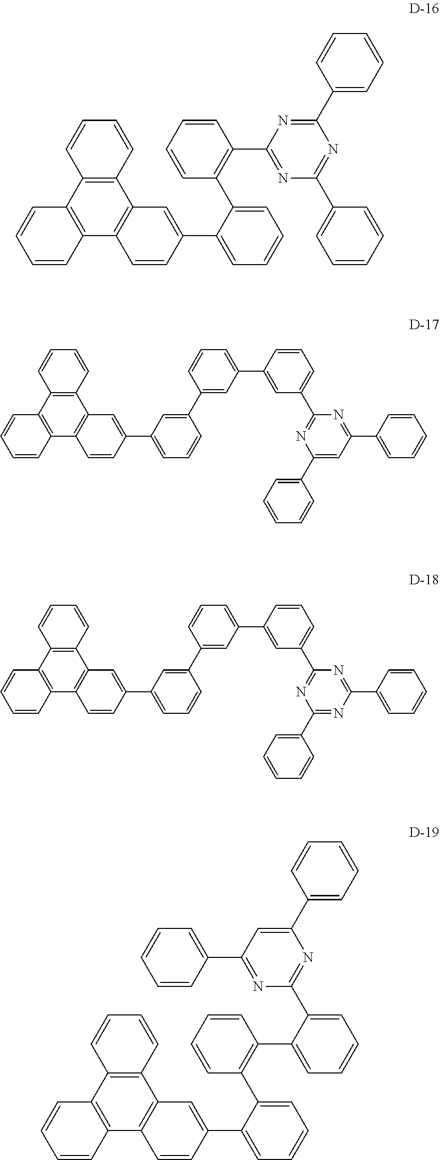 [SMILES: c1ccc(-c2nc(-c3ccccc3)nc(-c3ccccc3-c3ccccc3-c3ccc4c5ccccc5c5ccccc5c4c3)n2)cc1 ; c1ccc(-c2cc(-c3ccccc3)nc(-c3cccc(-c4cccc(-c5cccc(-c6ccc7c8ccccc8c8ccccc8c7c6)c5)c4)c3)n2)cc1 ; c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1ccc(-c2cc(-c3ccccc3)nc(-c3ccccc3-c3ccccc3-c3ccccc3-c3ccc4c5ccccc5c5ccccc5c4c3)n2)cc1]

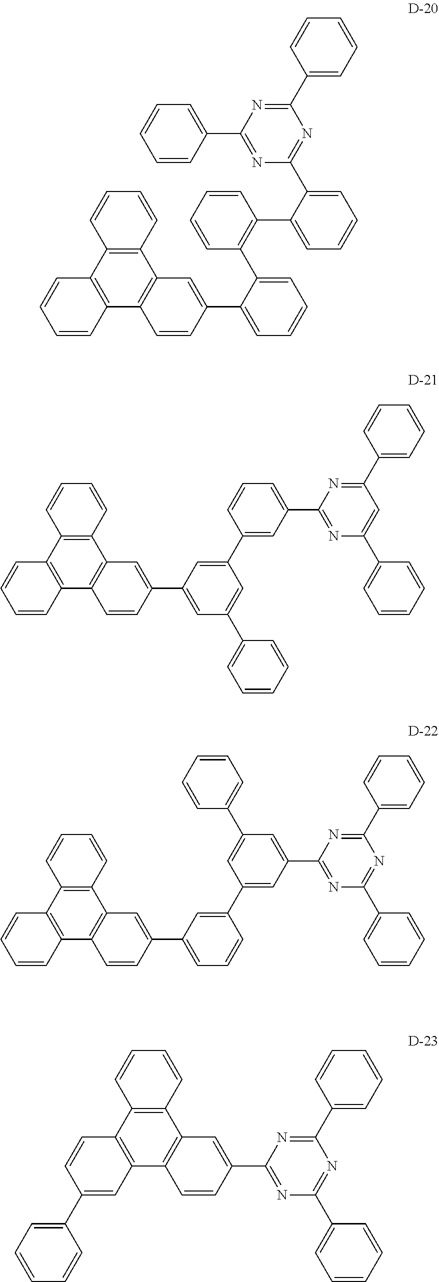 [SMILES: c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1 ; c1ccc(-c2cc(-c3cccc(-c4ccc5c6ccccc6c6ccccc6c5c4)c3)cc(-c3nc(-c4ccccc4)nc(-c4ccccc4)n3)c2)cc1 ; c1ccc(-c2ccc3c4ccccc4c4cc(-c5nc(-c6ccccc6)nc(-c6ccccc6)n5)ccc4c3c2)cc1]

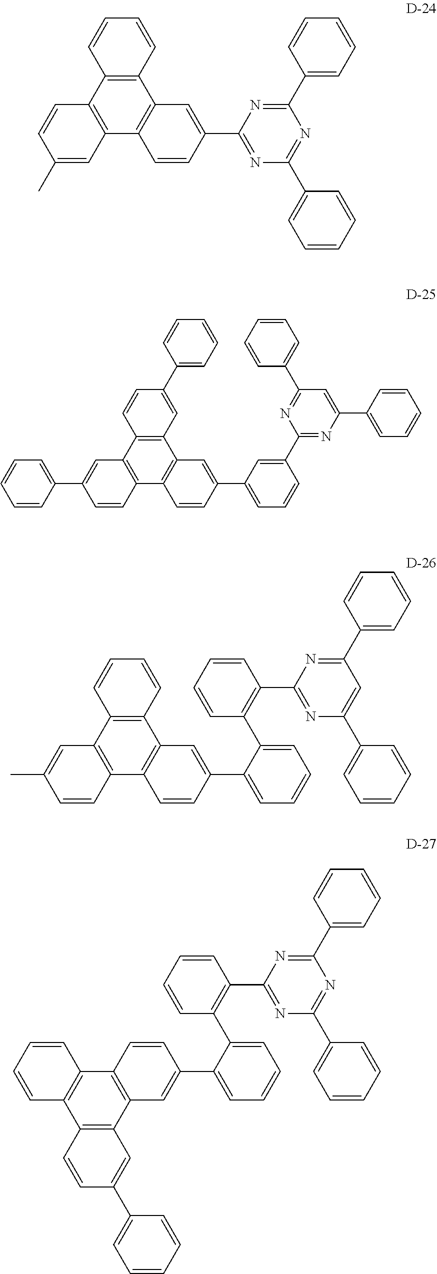 [SMILES: Cc1ccc2c3ccccc3c3cc(-c4nc(-c5ccccc5)nc(-c5ccccc5)n4)ccc3c2c1 ; c1ccc(-c2ccc3c4ccc(-c5cccc(-c6nc(-c7ccccc7)cc(-c7ccccc7)n6)c5)cc4c4cc(-c5ccccc5)ccc4c3c2)cc1 ; Cc1ccc2c3ccc(-c4ccccc4-c4ccccc4-c4nc(-c5ccccc5)cc(-c5ccccc5)n4)cc3c3ccccc3c2c1 ; c1ccc(-c2ccc3c4ccccc4c4ccc(-c5ccccc5-c5ccccc5-c5nc(-c6ccccc6)nc(-c6ccccc6)n5)cc4c3c2)cc1]

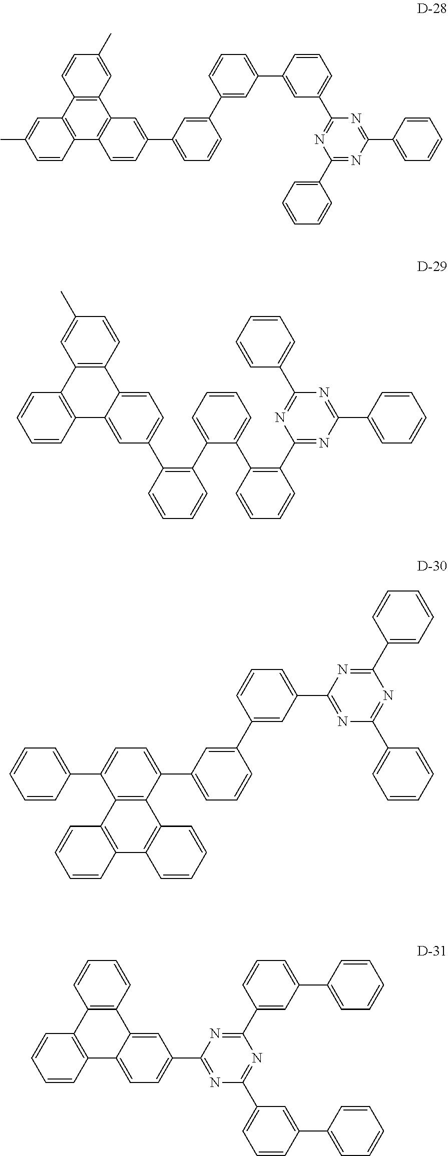 [SMILES: Cc1ccc2c3ccc(-c4cccc(-c5cccc(-c6cccc(-c7nc(-c8ccccc8)nc(-c8ccccc8)n7)c6)c5)c4)cc3c3cc(C)ccc3c2c1 ; Cc1ccc2c3ccc(-c4ccccc4-c4ccccc4-c4ccccc4-c4nc(-c5ccccc5)nc(-c5ccccc5)n4)cc3c3ccccc3c2c1 ; c1ccc(-c2nc(-c3ccccc3)nc(-c3cccc(-c4cccc(-c5ccc(-c6ccccc6)c6c7ccccc7c7ccccc7c56)c4)c3)n2)cc1 ; c1ccc(-c2cccc(-c3nc(-c4cccc(-c5ccccc5)c4)nc(-c4ccc5c6ccccc6c6ccccc6c5c4)n3)c2)cc1]

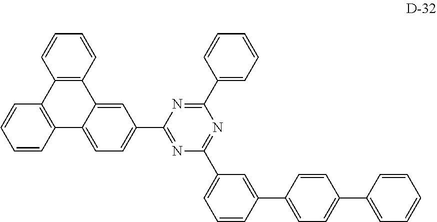 [SMILES: c1ccc(-c2ccc(-c3cccc(-c4nc(-c5ccccc5)nc(-c5ccc6c7ccccc7c7ccccc7c6c5)n4)c3)cc2)cc1]

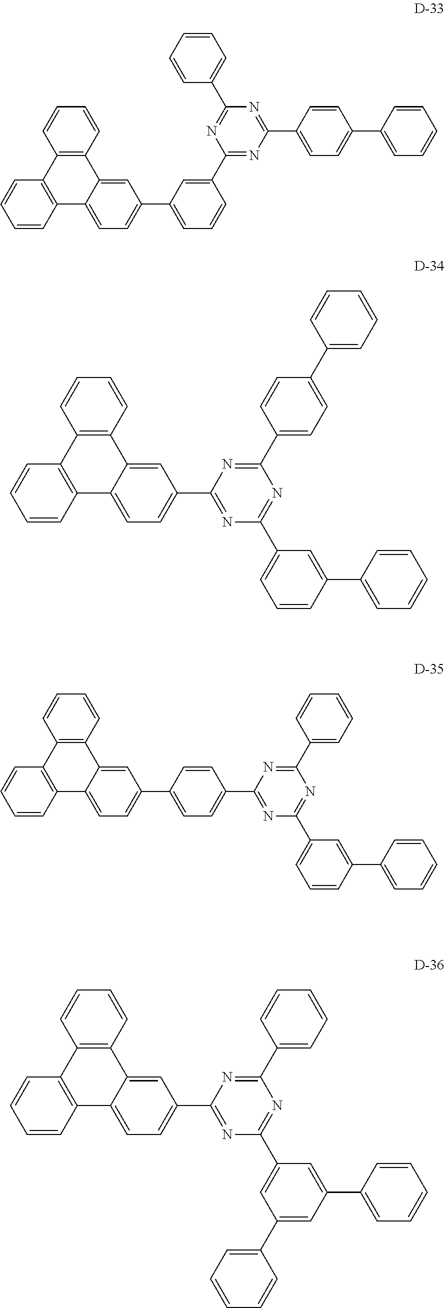 [SMILES: c1ccc(-c2ccc(-c3nc(-c4ccccc4)nc(-c4cccc(-c5ccc6c7ccccc7c7ccccc7c6c5)c4)n3)cc2)cc1 ; c1ccc(-c2ccc(-c3nc(-c4cccc(-c5ccccc5)c4)nc(-c4ccc5c6ccccc6c6ccccc6c5c4)n3)cc2)cc1 ; c1ccc(-c2cccc(-c3nc(-c4ccccc4)nc(-c4ccc(-c5ccc6c7ccccc7c7ccccc7c6c5)cc4)n3)c2)cc1 ; c1ccc(-c2cc(-c3ccccc3)cc(-c3nc(-c4ccccc4)nc(-c4ccc5c6ccccc6c6ccccc6c5c4)n3)c2)cc1]

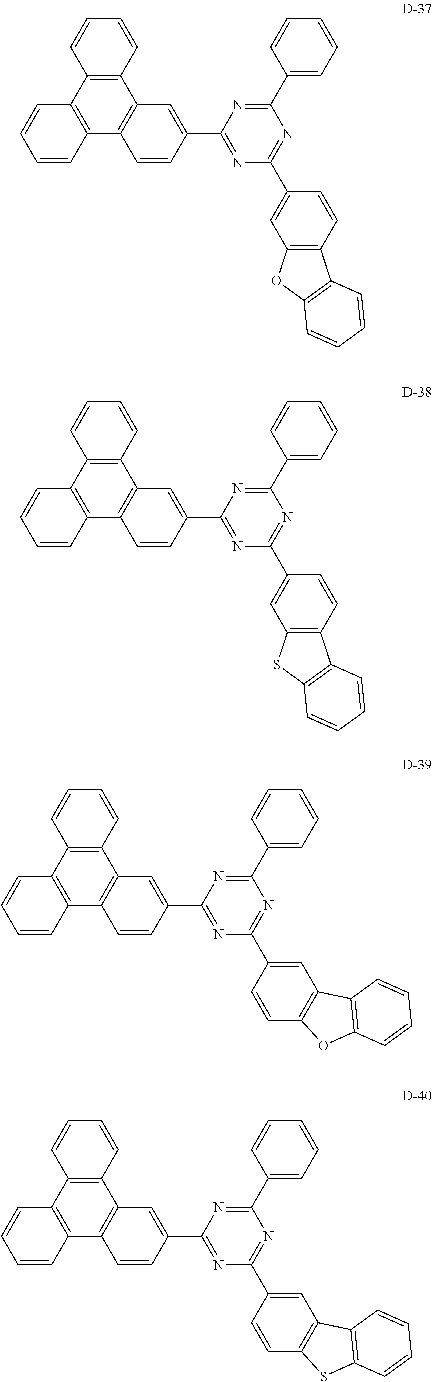 [SMILES: c1ccc(-c2nc(-c3ccc4c(c3)oc3ccccc34)nc(-c3ccc4c5ccccc5c5ccccc5c4c3)n2)cc1 ; c1ccc(-c2nc(-c3ccc4c(c3)sc3ccccc34)nc(-c3ccc4c5ccccc5c5ccccc5c4c3)n2)cc1 ; c1ccc(-c2nc(-c3ccc4oc5ccccc5c4c3)nc(-c3ccc4c5ccccc5c5ccccc5c4c3)n2)cc1 ; c1ccc(-c2nc(-c3ccc4sc5ccccc5c4c3)nc(-c3ccc4c5ccccc5c5ccccc5c4c3)n2)cc1]

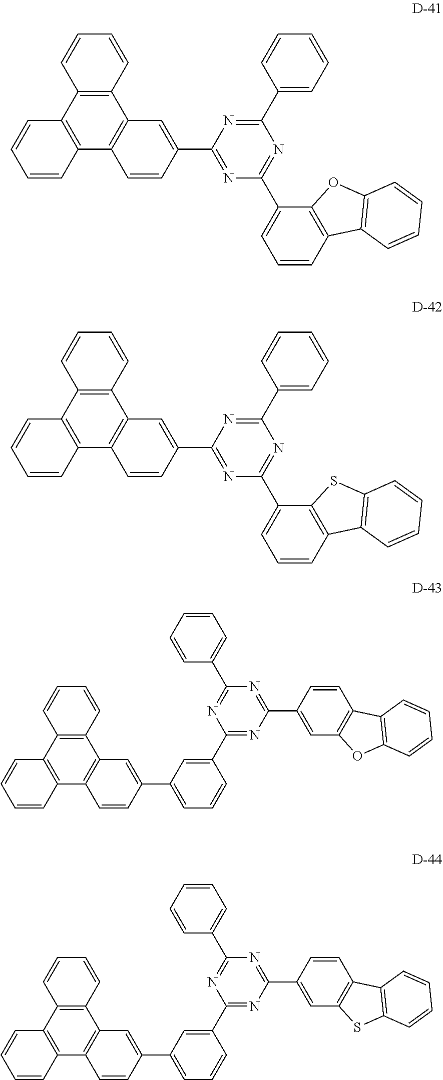 [SMILES: c1ccc(-c2nc(-c3ccc4c5ccccc5c5ccccc5c4c3)nc(-c3cccc4c3oc3ccccc34)n2)cc1 ; c1ccc(-c2nc(-c3ccc4c5ccccc5c5ccccc5c4c3)nc(-c3cccc4c3sc3ccccc34)n2)cc1 ; c1ccc(-c2nc(-c3cccc(-c4ccc5c6ccccc6c6ccccc6c5c4)c3)nc(-c3ccc4c(c3)oc3ccccc34)n2)cc1 ; c1ccc(-c2nc(-c3cccc(-c4ccc5c6ccccc6c6ccccc6c5c4)c3)nc(-c3ccc4c(c3)sc3ccccc34)n2)cc1]

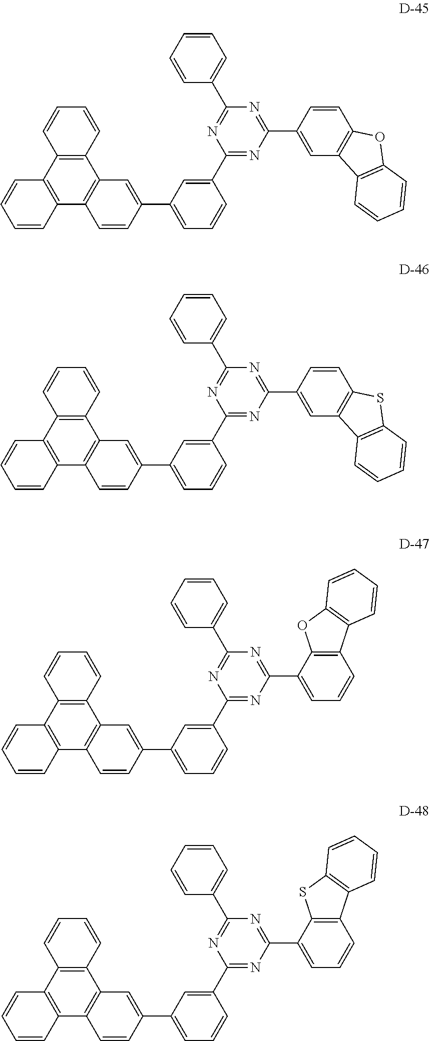 [SMILES: c1ccc(-c2nc(-c3cccc(-c4ccc5c6ccccc6c6ccccc6c5c4)c3)nc(-c3ccc4oc5ccccc5c4c3)n2)cc1 ; c1ccc(-c2nc(-c3cccc(-c4ccc5c6ccccc6c6ccccc6c5c4)c3)nc(-c3ccc4sc5ccccc5c4c3)n2)cc1 ; c1ccc(-c2nc(-c3cccc(-c4ccc5c6ccccc6c6ccccc6c5c4)c3)nc(-c3cccc4c3oc3ccccc34)n2)cc1 ; c1ccc(-c2nc(-c3cccc(-c4ccc5c6ccccc6c6ccccc6c5c4)c3)nc(-c3cccc4c3sc3ccccc34)n2)cc1]

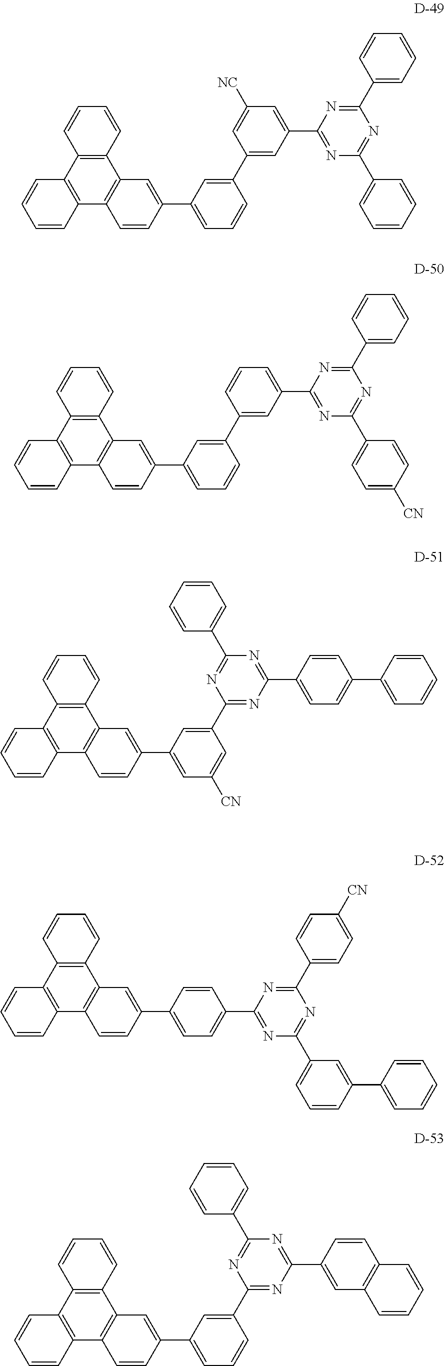 [SMILES: N#Cc1cc(-c2cccc(-c3ccc4c5ccccc5c5ccccc5c4c3)c2)cc(-c2nc(-c3ccccc3)nc(-c3ccccc3)n2)c1 ; N#Cc1ccc(-c2nc(-c3ccccc3)nc(-c3cccc(-c4cccc(-c5ccc6c7ccccc7c7ccccc7c6c5)c4)c3)n2)cc1 ; N#Cc1cc(-c2ccc3c4ccccc4c4ccccc4c3c2)cc(-c2nc(-c3ccccc3)nc(-c3ccc(-c4ccccc4)cc3)n2)c1 ; N#Cc1ccc(-c2nc(-c3ccc(-c4ccc5c6ccccc6c6ccccc6c5c4)cc3)nc(-c3cccc(-c4ccccc4)c3)n2)cc1 ; c1ccc(-c2nc(-c3cccc(-c4ccc5c6ccccc6c6ccccc6c5c4)c3)nc(-c3ccc4ccccc4c3)n2)cc1]

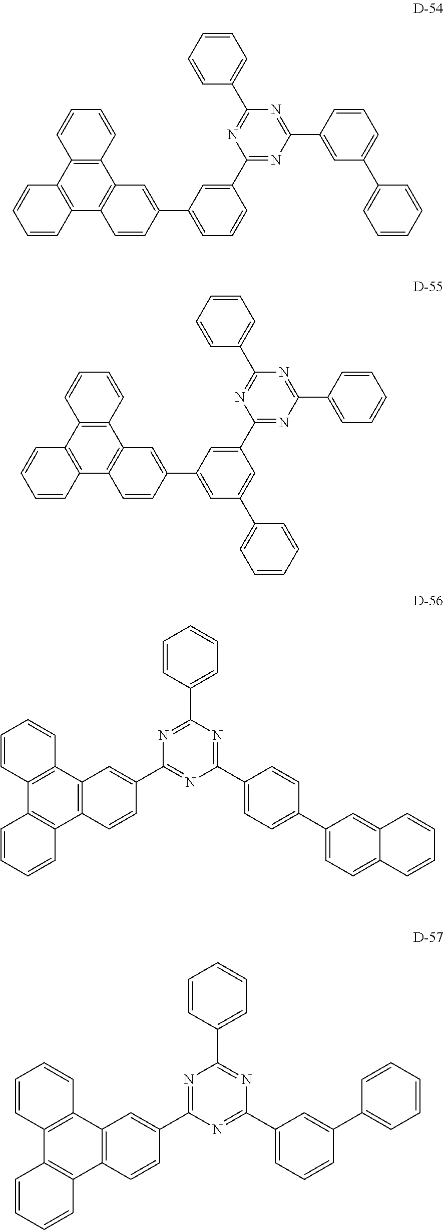 [SMILES: c1ccc(-c2cccc(-c3nc(-c4ccccc4)nc(-c4cccc(-c5ccc6c7ccccc7c7ccccc7c6c5)c4)n3)c2)cc1 ; c1ccc(-c2cc(-c3ccc4c5ccccc5c5ccccc5c4c3)cc(-c3nc(-c4ccccc4)nc(-c4ccccc4)n3)c2)cc1 ; c1ccc(-c2nc(-c3ccc(-c4ccc5ccccc5c4)cc3)nc(-c3ccc4c5ccccc5c5ccccc5c4c3)n2)cc1 ; c1ccc(-c2cccc(-c3nc(-c4ccccc4)nc(-c4ccc5c6ccccc6c6ccccc6c5c4)n3)c2)cc1]

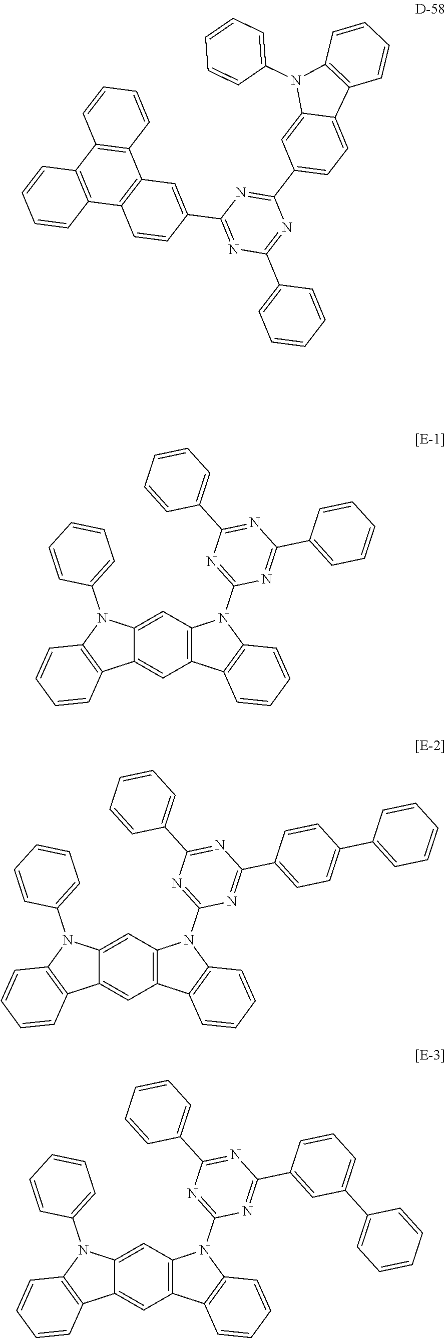 [SMILES: c1ccc(-c2nc(-c3ccc4c5ccccc5c5ccccc5c4c3)nc(-c3ccc4c5ccccc5n(-c5ccccc5)c4c3)n2)cc1 ; c1ccc(-c2nc(-c3ccccc3)nc(-n3c4ccccc4c4cc5c6ccccc6n(-c6ccccc6)c5cc43)n2)cc1 ; c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1]

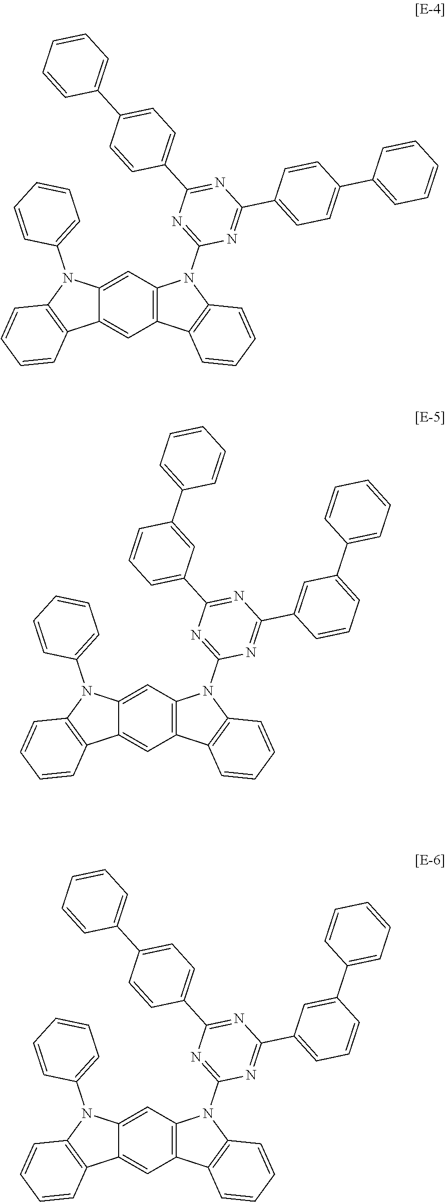 [SMILES: c1ccc(-c2ccc(-c3nc(-c4ccc(-c5ccccc5)cc4)nc(-n4c5ccccc5c5cc6c7ccccc7n(-c7ccccc7)c6cc54)n3)cc2)cc1 ; c1ccc(-c2cccc(-c3nc(-c4cccc(-c5ccccc5)c4)nc(-n4c5ccccc5c5cc6c7ccccc7n(-c7ccccc7)c6cc54)n3)c2)cc1 ; c1ccc(-c2ccc(-c3nc(-c4cccc(-c5ccccc5)c4)nc(-n4c5ccccc5c5cc6c7ccccc7n(-c7ccccc7)c6cc54)n3)cc2)cc1]

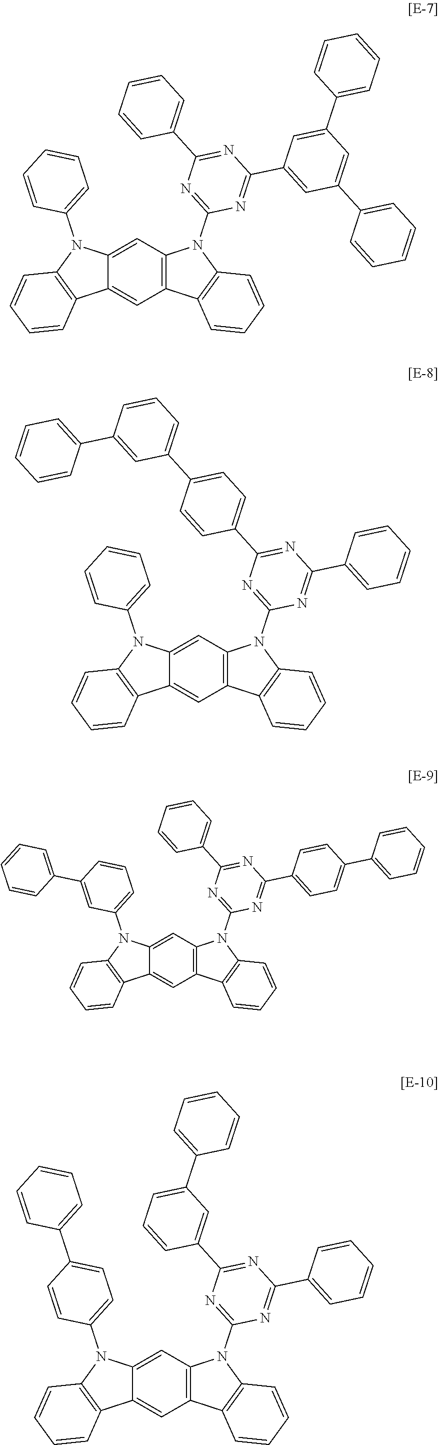 [SMILES: c1ccc(-c2cc(-c3ccccc3)cc(-c3nc(-c4ccccc4)nc(-n4c5ccccc5c5cc6c7ccccc7n(-c7ccccc7)c6cc54)n3)c2)cc1 ; c1ccc(-c2cccc(-c3ccc(-c4nc(-c5ccccc5)nc(-n5c6ccccc6c6cc7c8ccccc8n(-c8ccccc8)c7cc65)n4)cc3)c2)cc1 ; c1ccc(-c2ccc(-c3nc(-c4ccccc4)nc(-n4c5ccccc5c5cc6c7ccccc7n(-c7cccc(-c8ccccc8)c7)c6cc54)n3)cc2)cc1 ; c1ccc(-c2ccc(-n3c4ccccc4c4cc5c6ccccc6n(-c6nc(-c7ccccc7)nc(-c7cccc(-c8ccccc8)c7)n6)c5cc43)cc2)cc1]

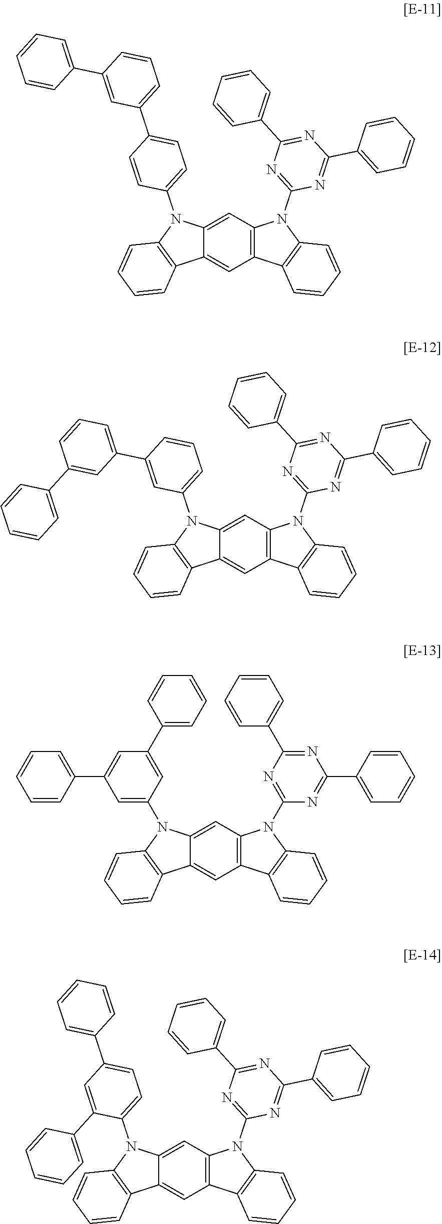 [SMILES: c1ccc(-c2cccc(-c3ccc(-n4c5ccccc5c5cc6c7ccccc7n(-c7nc(-c8ccccc8)nc(-c8ccccc8)n7)c6cc54)cc3)c2)cc1 ; c1ccc(-c2cccc(-c3cccc(-n4c5ccccc5c5cc6c7ccccc7n(-c7nc(-c8ccccc8)nc(-c8ccccc8)n7)c6cc54)c3)c2)cc1 ; c1ccc(-c2cc(-c3ccccc3)cc(-n3c4ccccc4c4cc5c6ccccc6n(-c6nc(-c7ccccc7)nc(-c7ccccc7)n6)c5cc43)c2)cc1 ; c1ccc(-c2ccc(-n3c4ccccc4c4cc5c6ccccc6n(-c6nc(-c7ccccc7)nc(-c7ccccc7)n6)c5cc43)c(-c3ccccc3)c2)cc1]

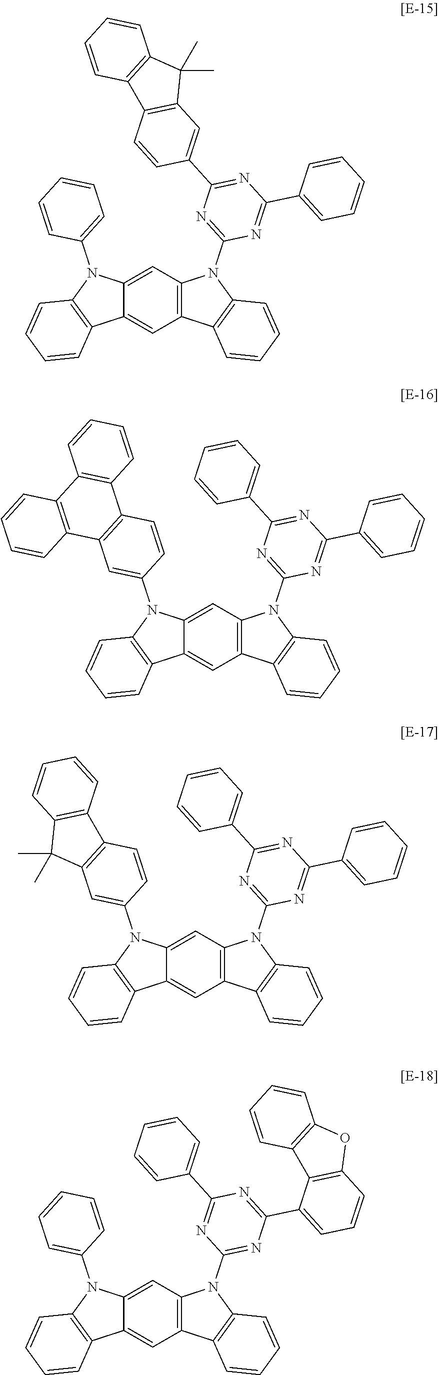 [SMILES: CC1(C)c2ccccc2-c2ccc(-c3nc(-c4ccccc4)nc(-n4c5ccccc5c5cc6c7ccccc7n(-c7ccccc7)c6cc54)n3)cc21 ; c1ccc(-c2nc(-c3ccccc3)nc(-n3c4ccccc4c4cc5c6ccccc6n(-c6ccc7c8ccccc8c8ccccc8c7c6)c5cc43)n2)cc1 ; CC1(C)c2ccccc2-c2ccc(-n3c4ccccc4c4cc5c6ccccc6n(-c6nc(-c7ccccc7)nc(-c7ccccc7)n6)c5cc43)cc21 ; c1ccc(-c2nc(-c3cccc4oc5ccccc5c34)nc(-n3c4ccccc4c4cc5c6ccccc6n(-c6ccccc6)c5cc43)n2)cc1]

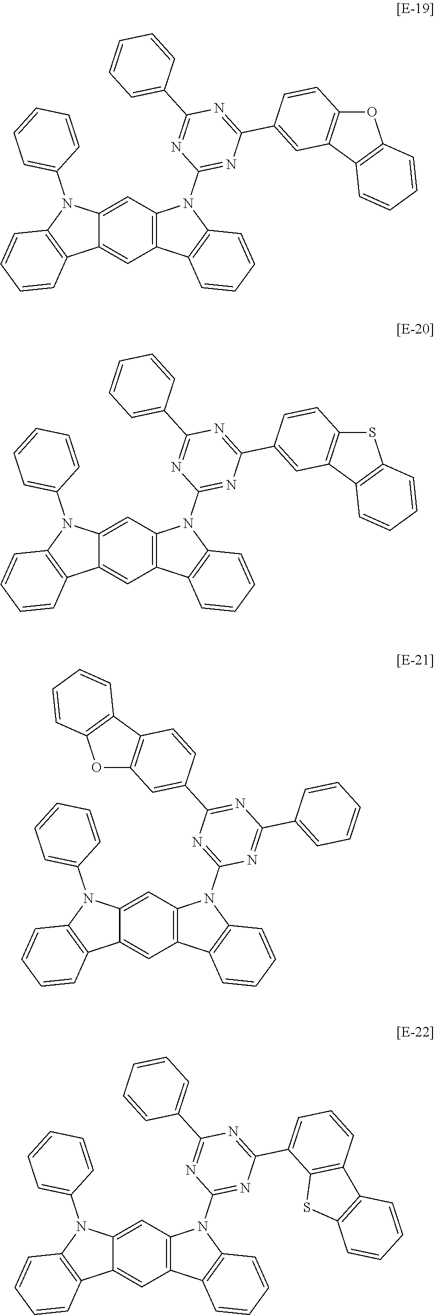 [SMILES: c1ccc(-c2nc(-c3ccc4oc5ccccc5c4c3)nc(-n3c4ccccc4c4cc5c6ccccc6n(-c6ccccc6)c5cc43)n2)cc1 ; c1ccc(-c2nc(-c3ccc4sc5ccccc5c4c3)nc(-n3c4ccccc4c4cc5c6ccccc6n(-c6ccccc6)c5cc43)n2)cc1 ; c1ccc(-c2nc(-c3ccc4c(c3)oc3ccccc34)nc(-n3c4ccccc4c4cc5c6ccccc6n(-c6ccccc6)c5cc43)n2)cc1 ; c1ccc(-c2nc(-c3cccc4c3sc3ccccc34)nc(-n3c4ccccc4c4cc5c6ccccc6n(-c6ccccc6)c5cc43)n2)cc1]

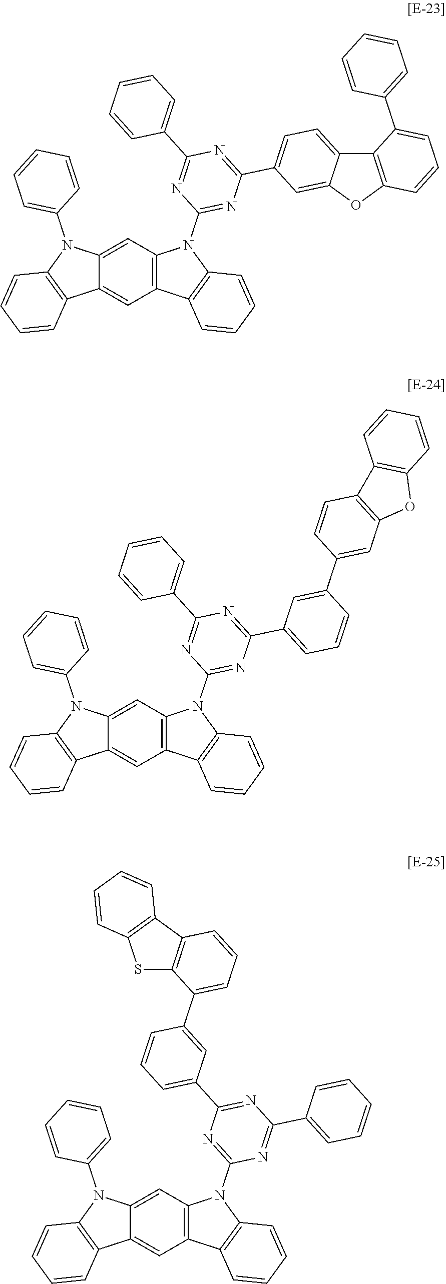 [SMILES: c1ccc(-c2nc(-c3ccc4c(c3)oc3cccc(-c5ccccc5)c34)nc(-n3c4ccccc4c4cc5c6ccccc6n(-c6ccccc6)c5cc43)n2)cc1 ; c1ccc(-c2nc(-c3cccc(-c4ccc5c(c4)oc4ccccc45)c3)nc(-n3c4ccccc4c4cc5c6ccccc6n(-c6ccccc6)c5cc43)n2)cc1 ; c1ccc(-c2nc(-c3cccc(-c4cccc5c4sc4ccccc45)c3)nc(-n3c4ccccc4c4cc5c6ccccc6n(-c6ccccc6)c5cc43)n2)cc1]

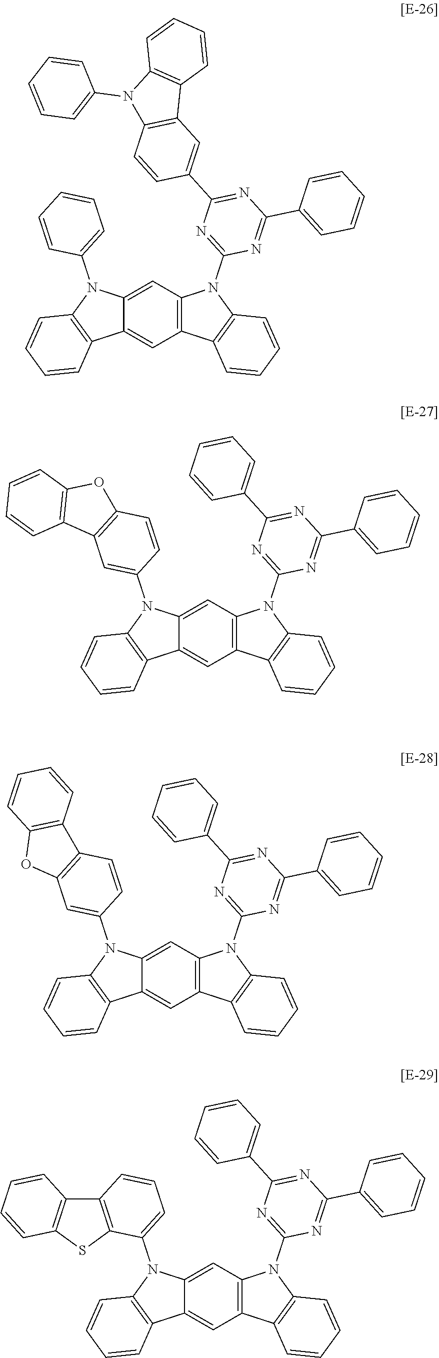 [SMILES: c1ccc(-c2nc(-c3ccc4c(c3)c3ccccc3n4-c3ccccc3)nc(-n3c4ccccc4c4cc5c6ccccc6n(-c6ccccc6)c5cc43)n2)cc1 ; c1ccc(-c2nc(-c3ccccc3)nc(-n3c4ccccc4c4cc5c6ccccc6n(-c6ccc7oc8ccccc8c7c6)c5cc43)n2)cc1 ; c1ccc(-c2nc(-c3ccccc3)nc(-n3c4ccccc4c4cc5c6ccccc6n(-c6ccc7c(c6)oc6ccccc67)c5cc43)n2)cc1 ; c1ccc(-c2nc(-c3ccccc3)nc(-n3c4ccccc4c4cc5c6ccccc6n(-c6cccc7c6sc6ccccc67)c5cc43)n2)cc1]

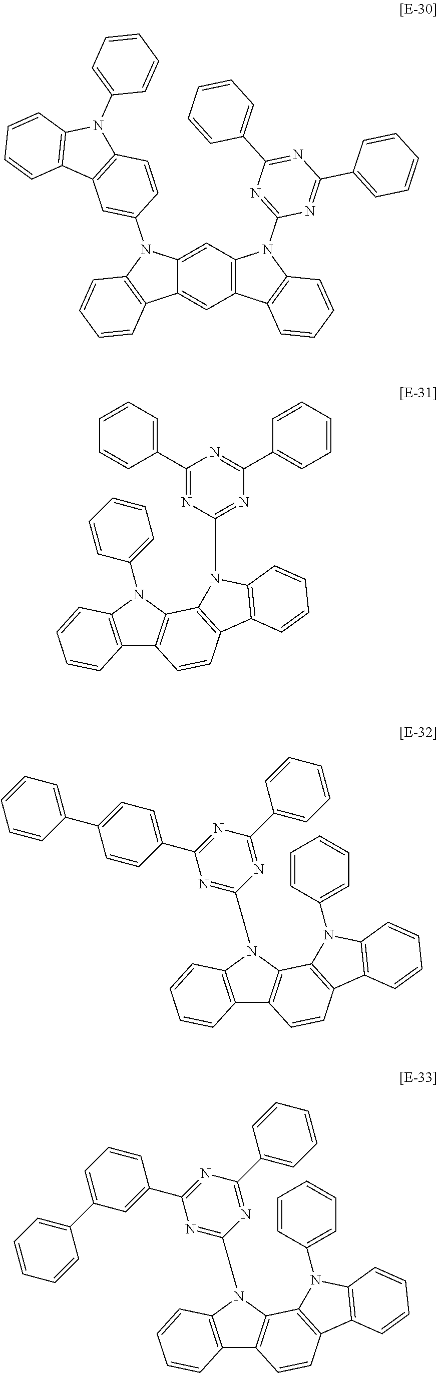 [SMILES: c1ccc(-c2nc(-c3ccccc3)nc(-n3c4ccccc4c4cc5c6ccccc6n(-c6ccc7c(c6)c6ccccc6n7-c6ccccc6)c5cc43)n2)cc1 ; c1ccc(-c2nc(-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2cccc(-c3nc(-c4ccccc4)nc(-n4c5ccccc5c5ccc6c7ccccc7n(-c7ccccc7)c6c54)n3)c2)cc1]

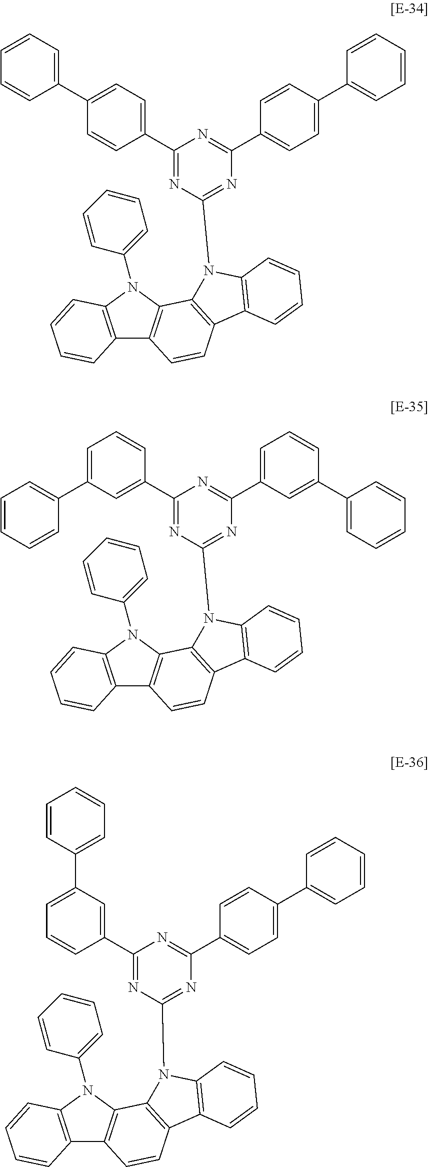 [SMILES: c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1 ; c1ccc(-c2cccc(-c3nc(-c4cccc(-c5ccccc5)c4)nc(-n4c5ccccc5c5ccc6c7ccccc7n(-c7ccccc7)c6c54)n3)c2)cc1 ; c1ccc(-c2ccc(-c3nc(-c4cccc(-c5ccccc5)c4)nc(-n4c5ccccc5c5ccc6c7ccccc7n(-c7ccccc7)c6c54)n3)cc2)cc1]

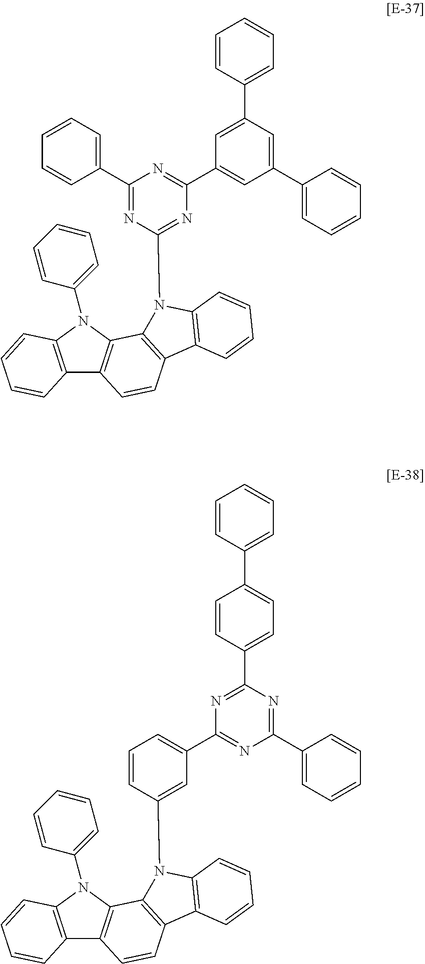 [SMILES: c1ccc(-c2cc(-c3ccccc3)cc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccccc4)nc(-c4cccc(-n5c6ccccc6c6ccc7c8ccccc8n(-c8ccccc8)c7c65)c4)n3)cc2)cc1]

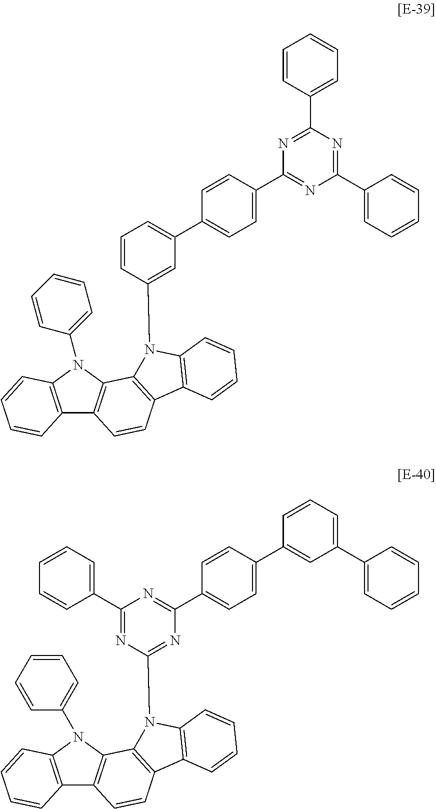 [SMILES: c1ccc(-c2nc(-c3ccccc3)nc(-c3ccc(-c4cccc(-n5c6ccccc6c6ccc7c8ccccc8n(-c8ccccc8)c7c65)c4)cc3)n2)cc1 ; c1ccc(-c2cccc(-c3ccc(-c4nc(-c5ccccc5)nc(-n5c6ccccc6c6ccc7c8ccccc8n(-c8ccccc8)c7c65)n4)cc3)c2)cc1]

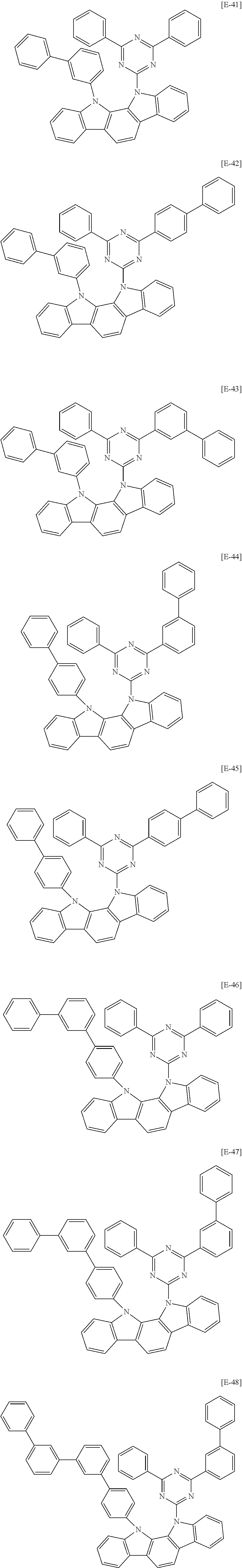

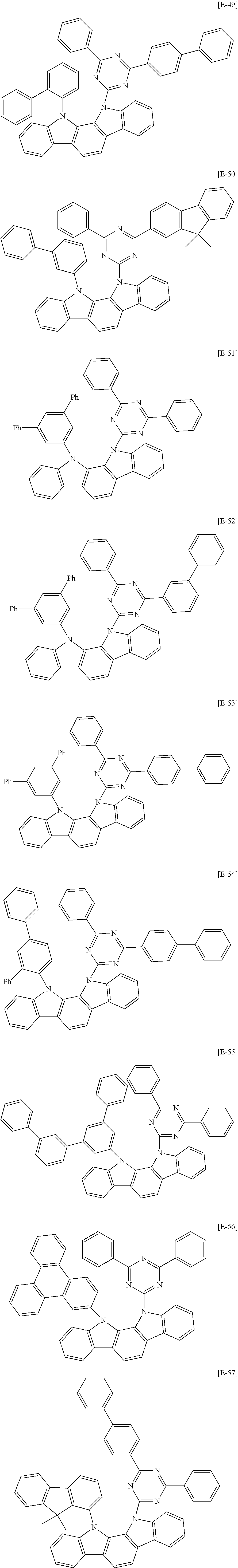

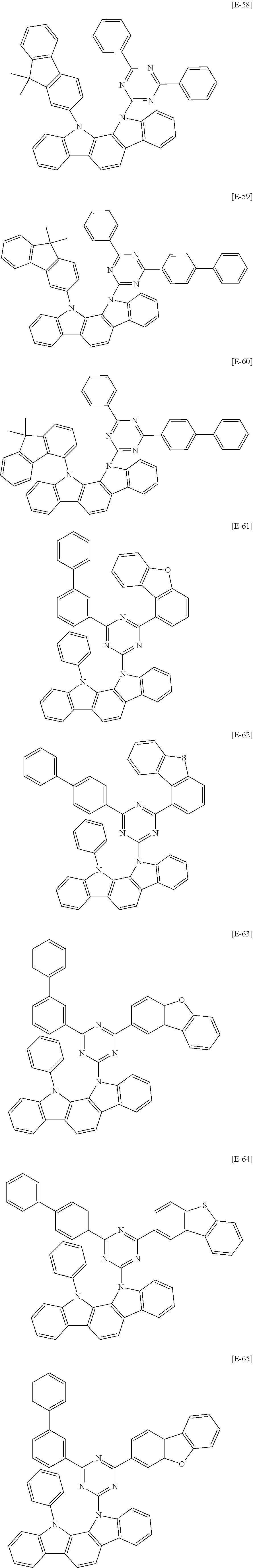

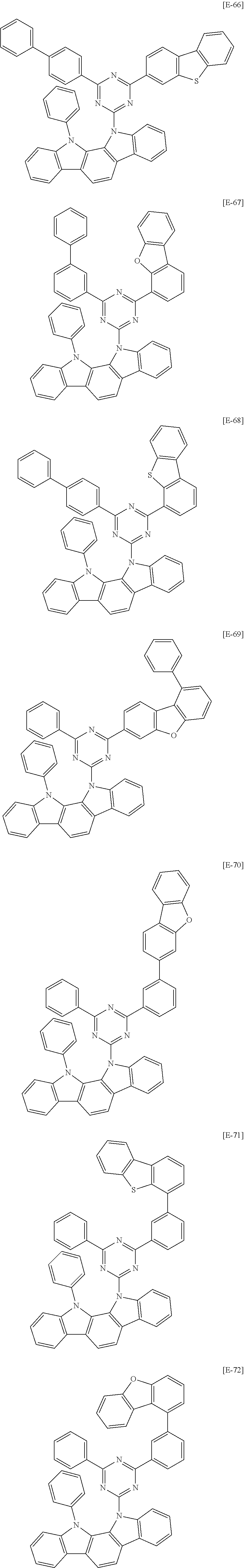

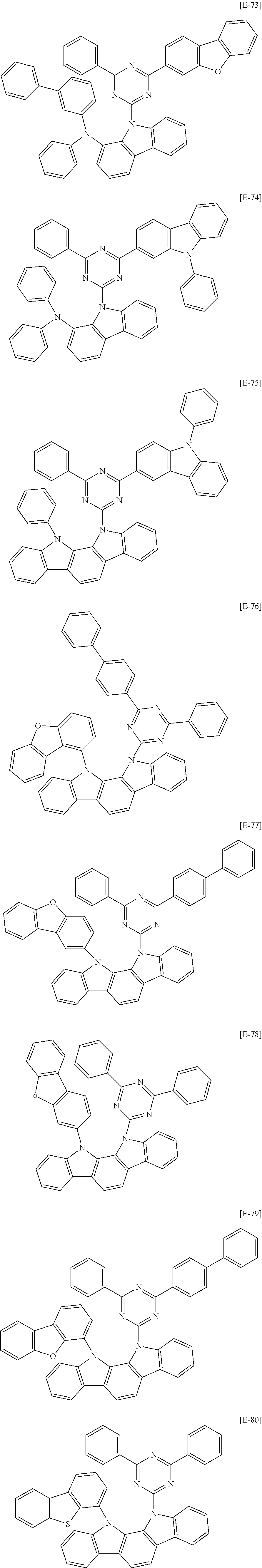

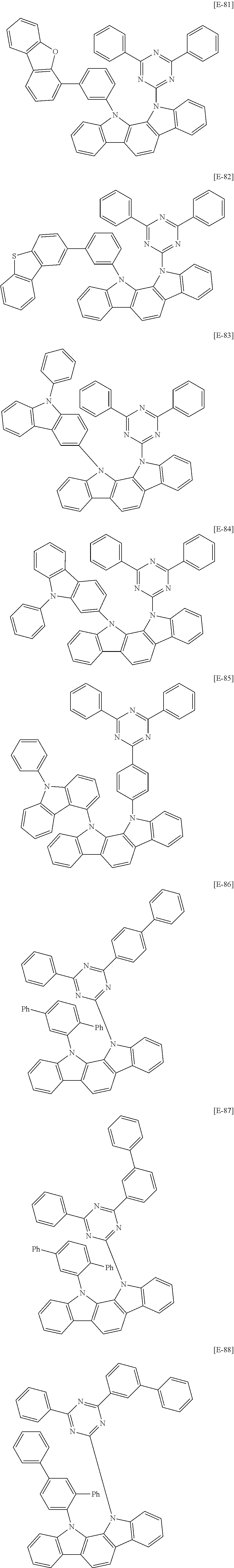

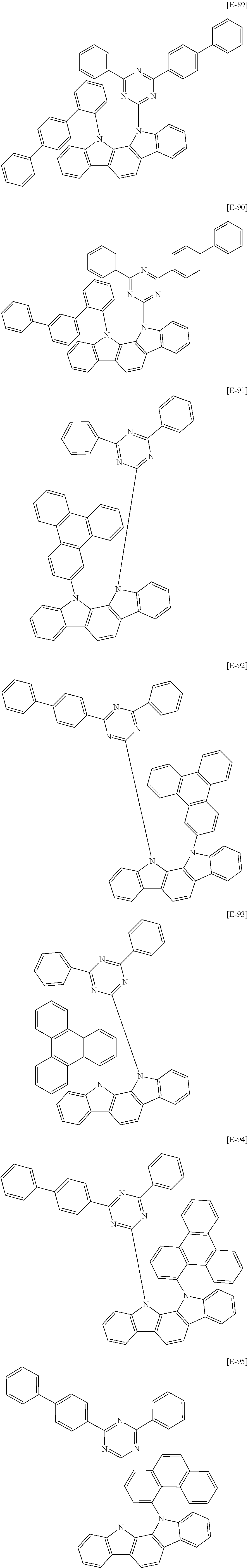

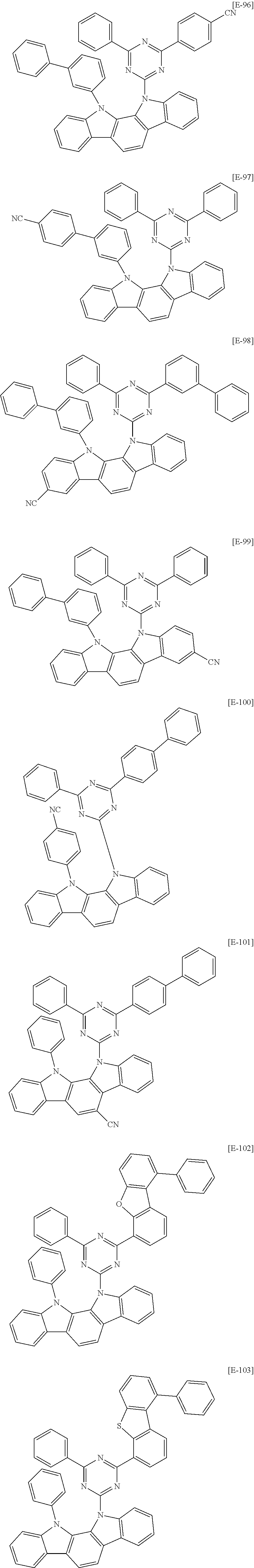

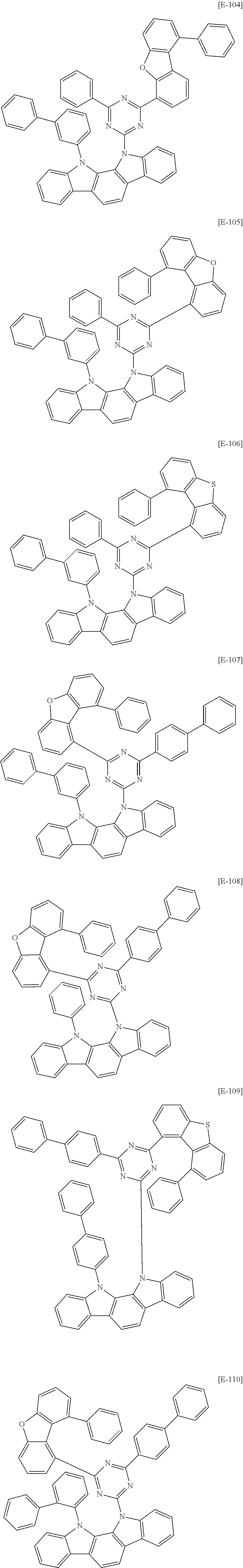

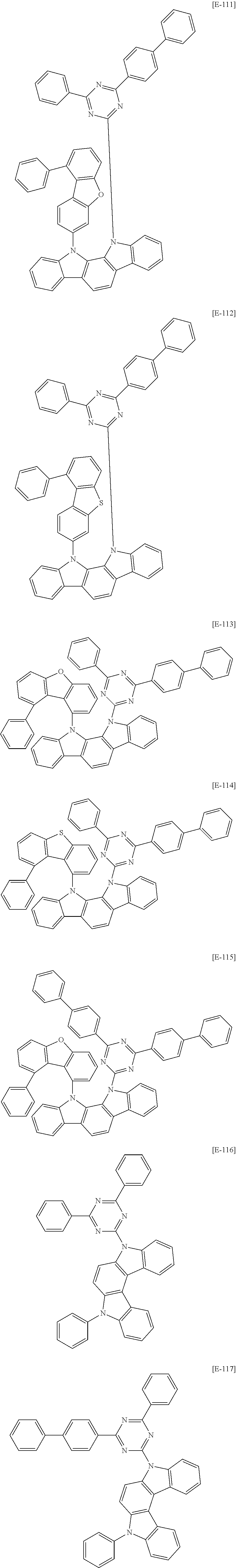

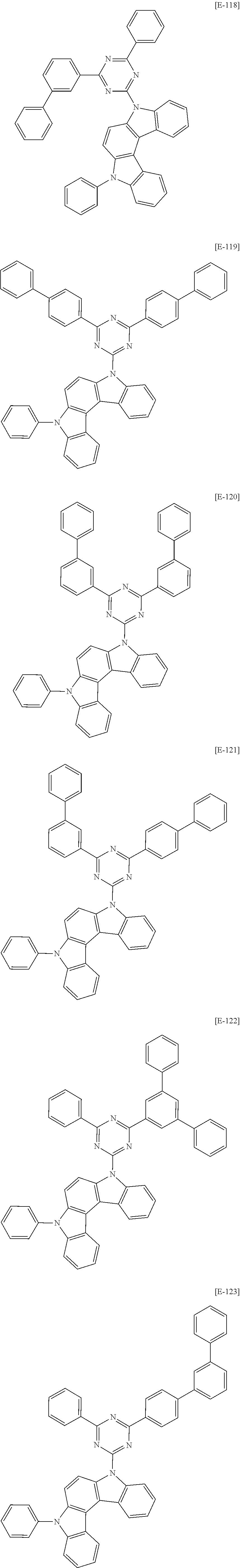

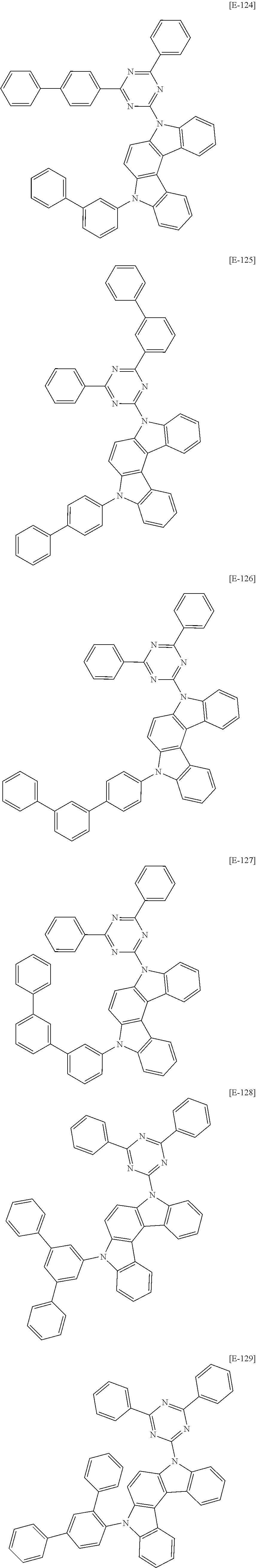

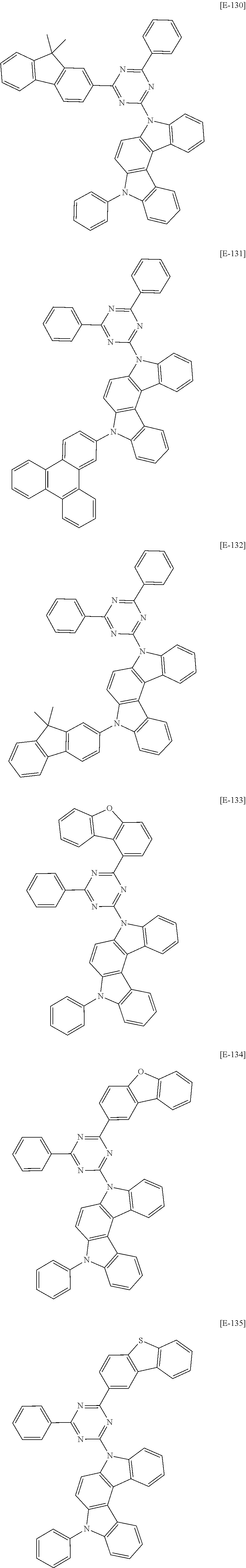

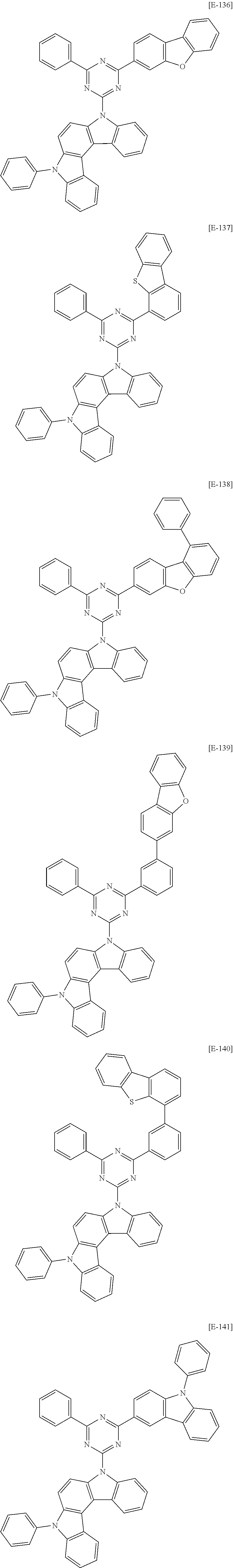

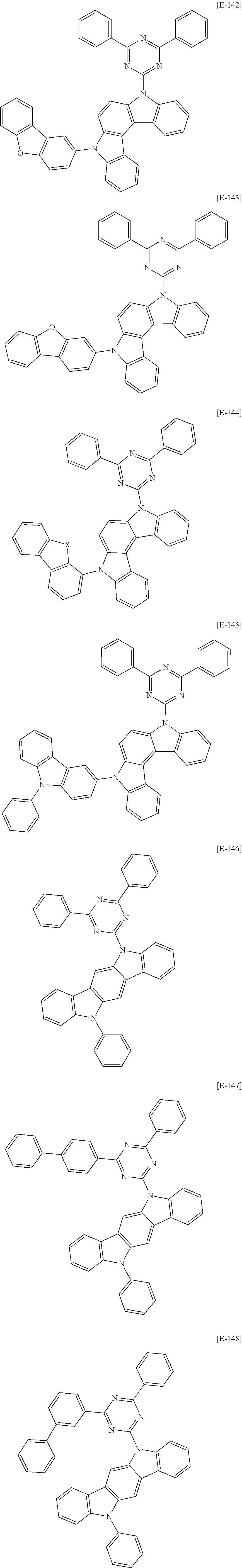

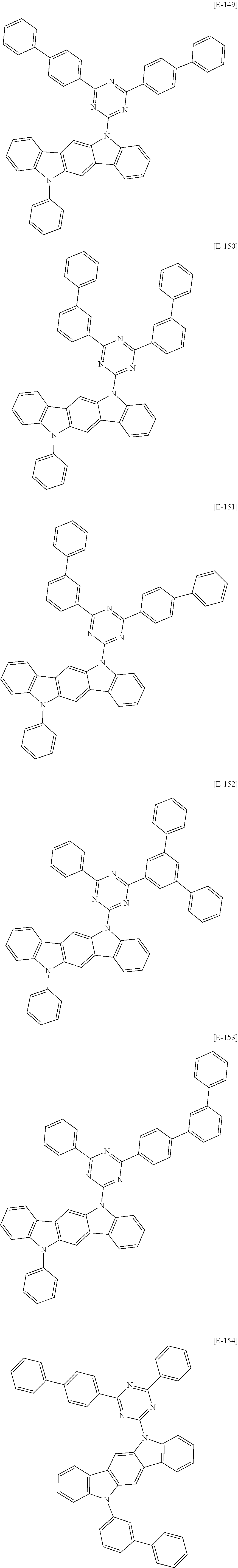

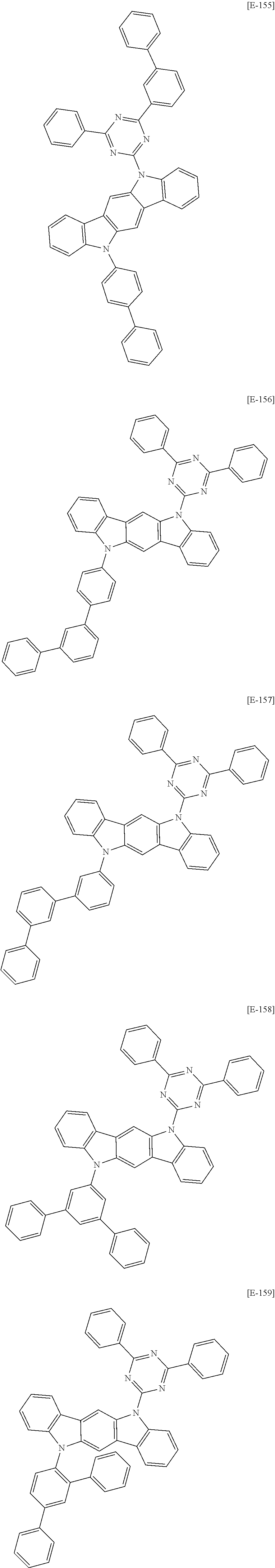 [SMILES: c1ccc(-c2ccc(-n3c4ccccc4c4cc5c(cc43)c3ccccc3n5-c3nc(-c4ccccc4)nc(-c4cccc(-c5ccccc5)c4)n3)cc2)cc1 ; c1ccc(-c2cccc(-c3ccc(-n4c5ccccc5c5cc6c(cc54)c4ccccc4n6-c4nc(-c5ccccc5)nc(-c5ccccc5)n4)cc3)c2)cc1 ; c1ccc(-c2cccc(-c3cccc(-n4c5ccccc5c5cc6c(cc54)c4ccccc4n6-c4nc(-c5ccccc5)nc(-c5ccccc5)n4)c3)c2)cc1 ; c1ccc(-c2cc(-c3ccccc3)cc(-n3c4ccccc4c4cc5c(cc43)c3ccccc3n5-c3nc(-c4ccccc4)nc(-c4ccccc4)n3)c2)cc1 ; c1ccc(-c2ccc(-n3c4ccccc4c4cc5c(cc43)c3ccccc3n5-c3nc(-c4ccccc4)nc(-c4ccccc4)n3)c(-c3ccccc3)c2)cc1]

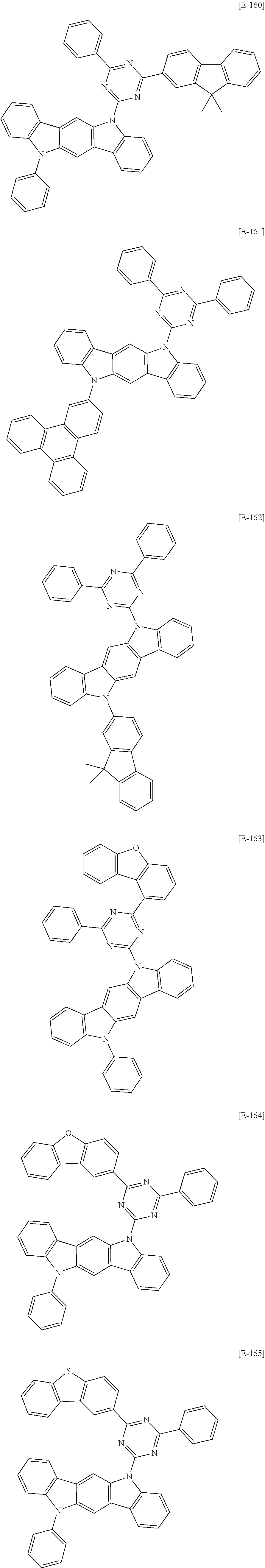

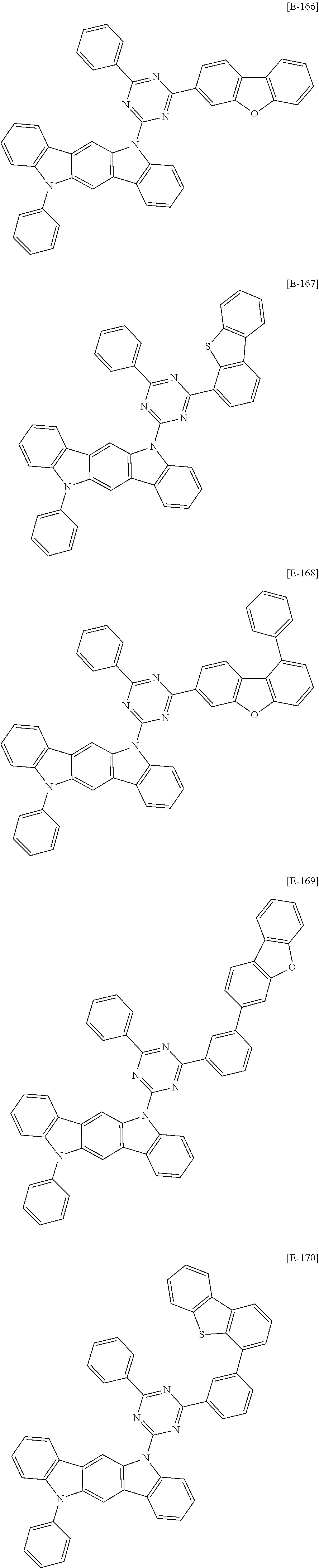 [SMILES: c1ccc(-c2nc(-c3ccc4c(c3)oc3ccccc34)nc(-n3c4ccccc4c4cc5c(cc43)c3ccccc3n5-c3ccccc3)n2)cc1 ; c1ccc(-c2nc(-c3cccc4c3sc3ccccc34)nc(-n3c4ccccc4c4cc5c(cc43)c3ccccc3n5-c3ccccc3)n2)cc1 ; c1ccc(-c2nc(-c3ccc4c(c3)oc3cccc(-c5ccccc5)c34)nc(-n3c4ccccc4c4cc5c(cc43)c3ccccc3n5-c3ccccc3)n2)cc1 ; c1ccc(-c2nc(-c3cccc(-c4ccc5c(c4)oc4ccccc45)c3)nc(-n3c4ccccc4c4cc5c(cc43)c3ccccc3n5-c3ccccc3)n2)cc1 ; c1ccc(-c2nc(-c3cccc(-c4cccc5c4sc4ccccc45)c3)nc(-n3c4ccccc4c4cc5c(cc43)c3ccccc3n5-c3ccccc3)n2)cc1]

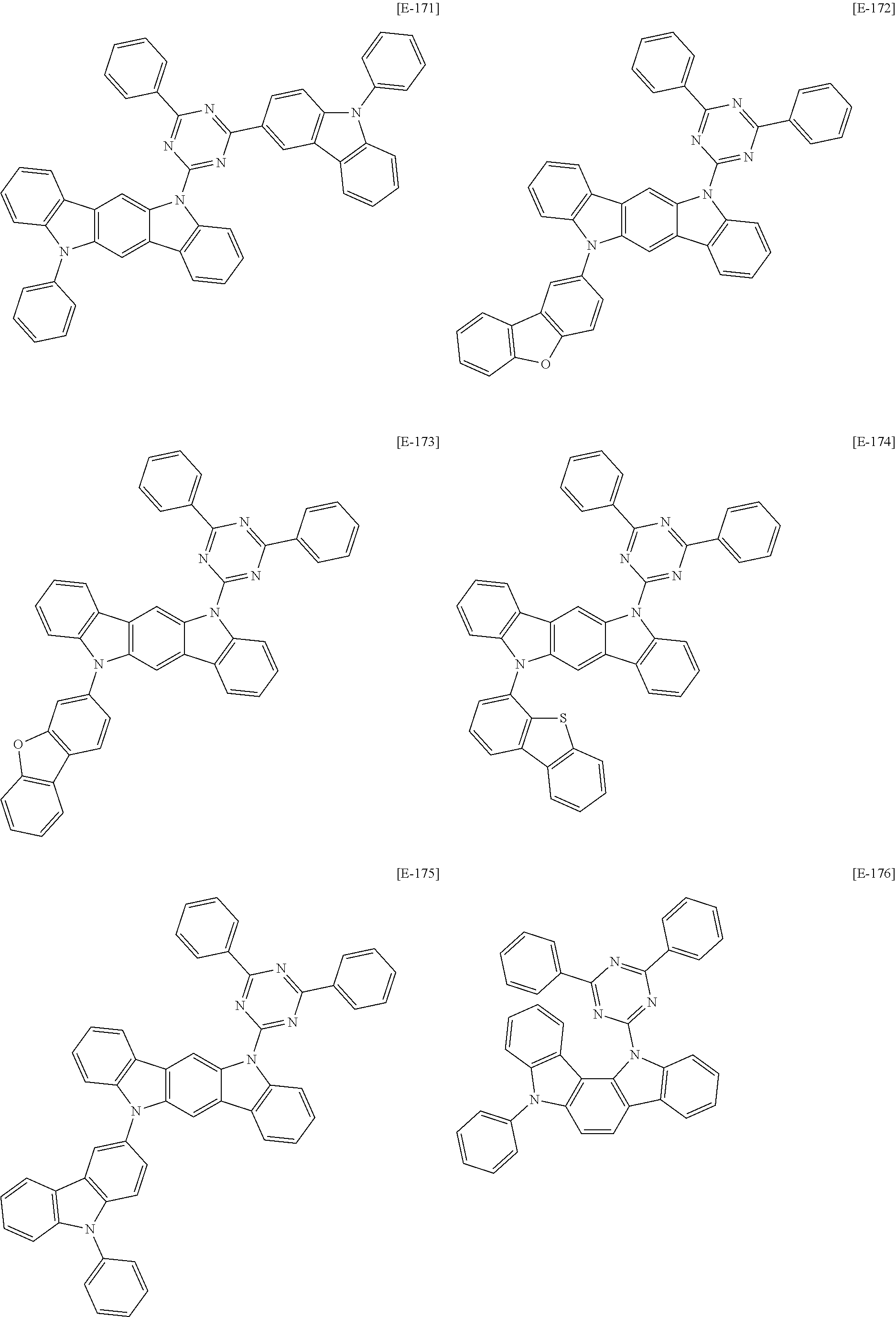

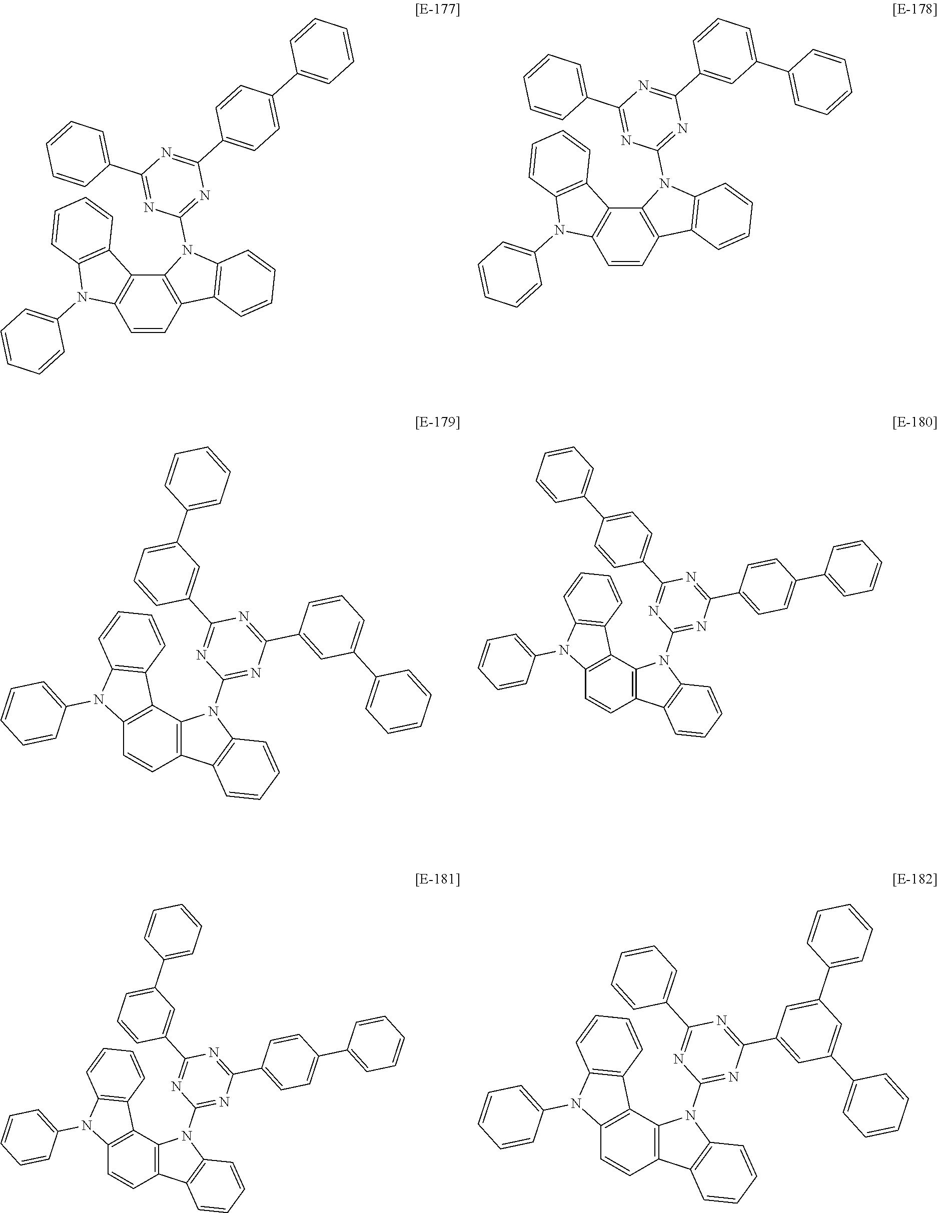

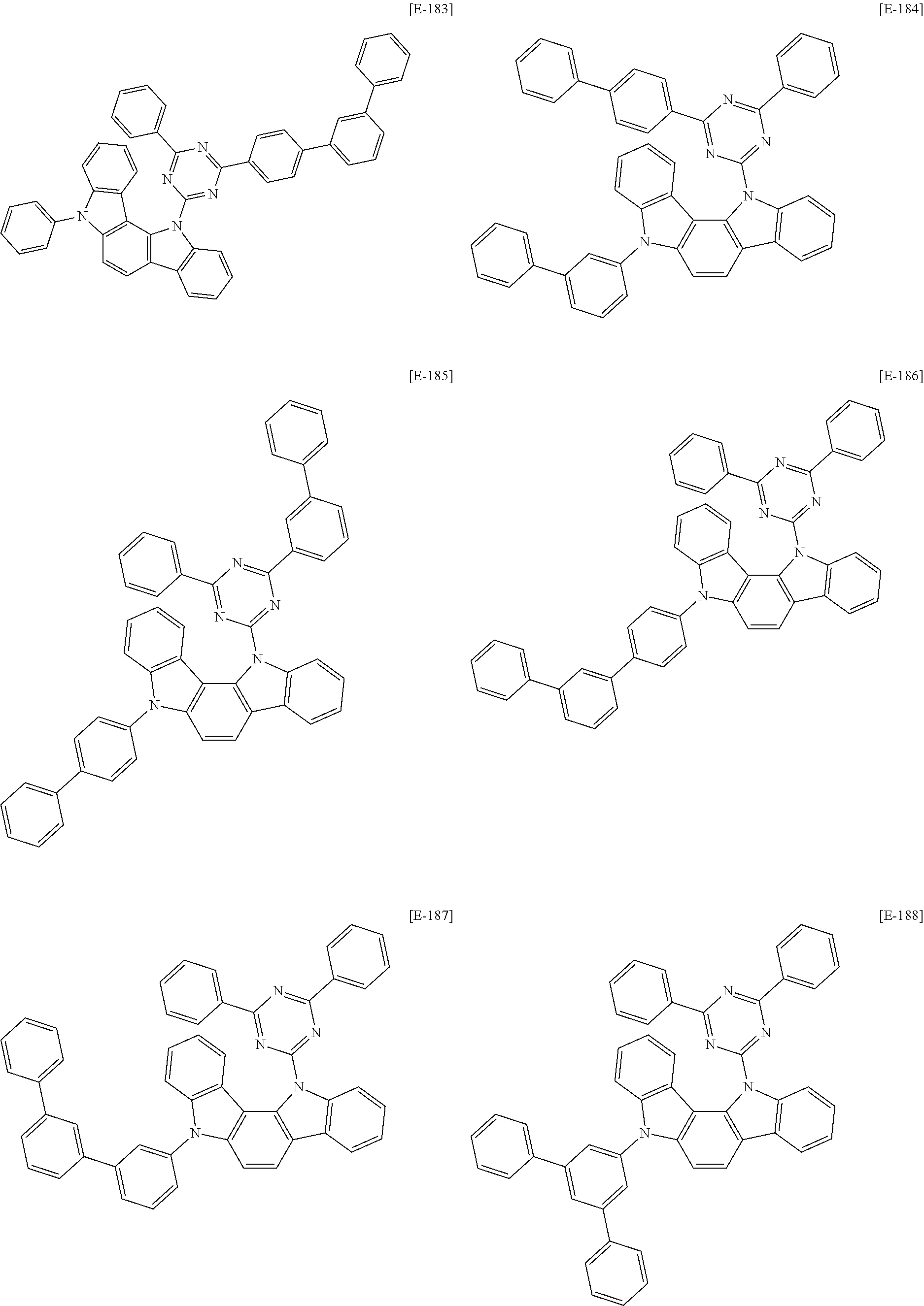

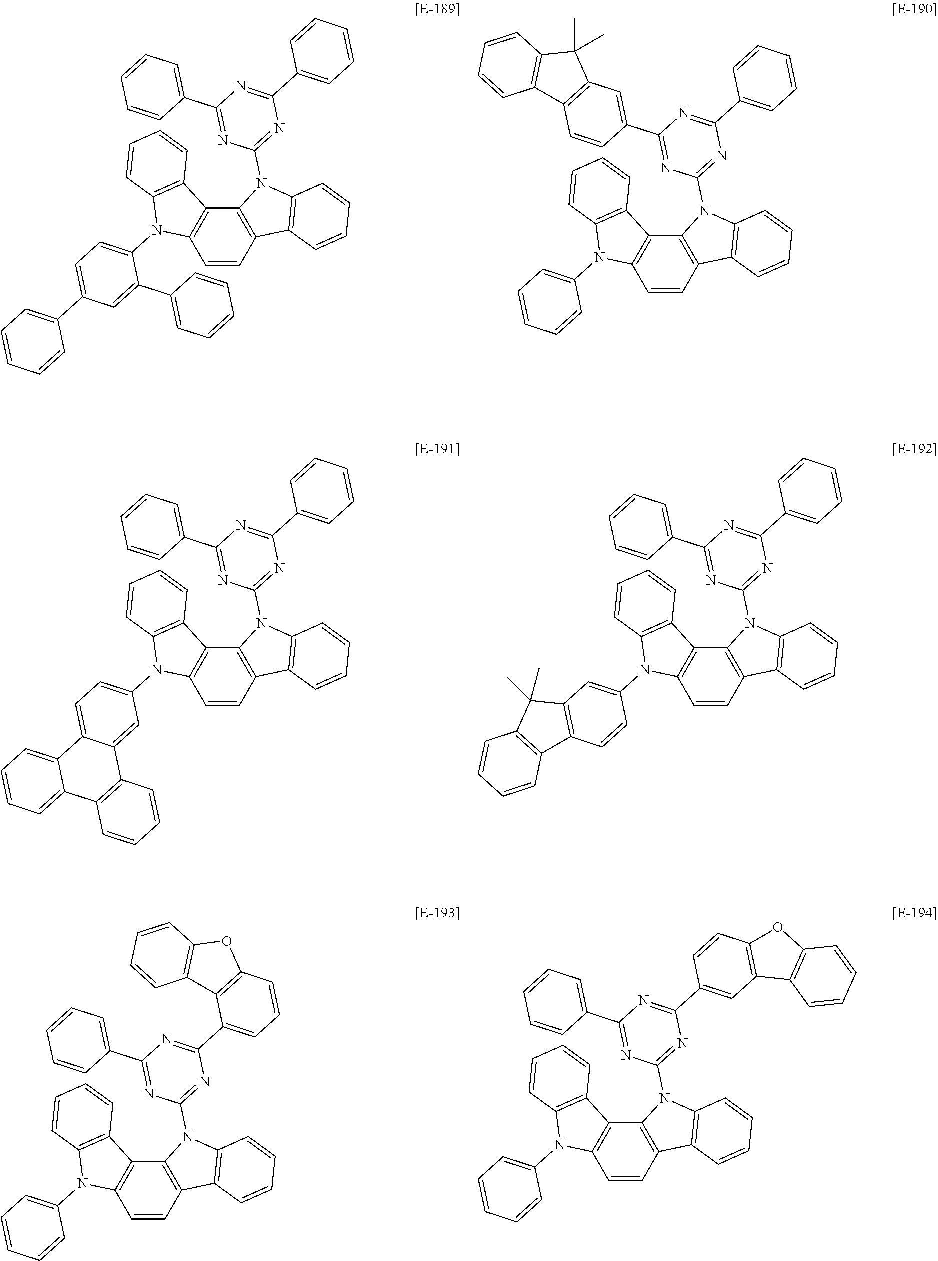

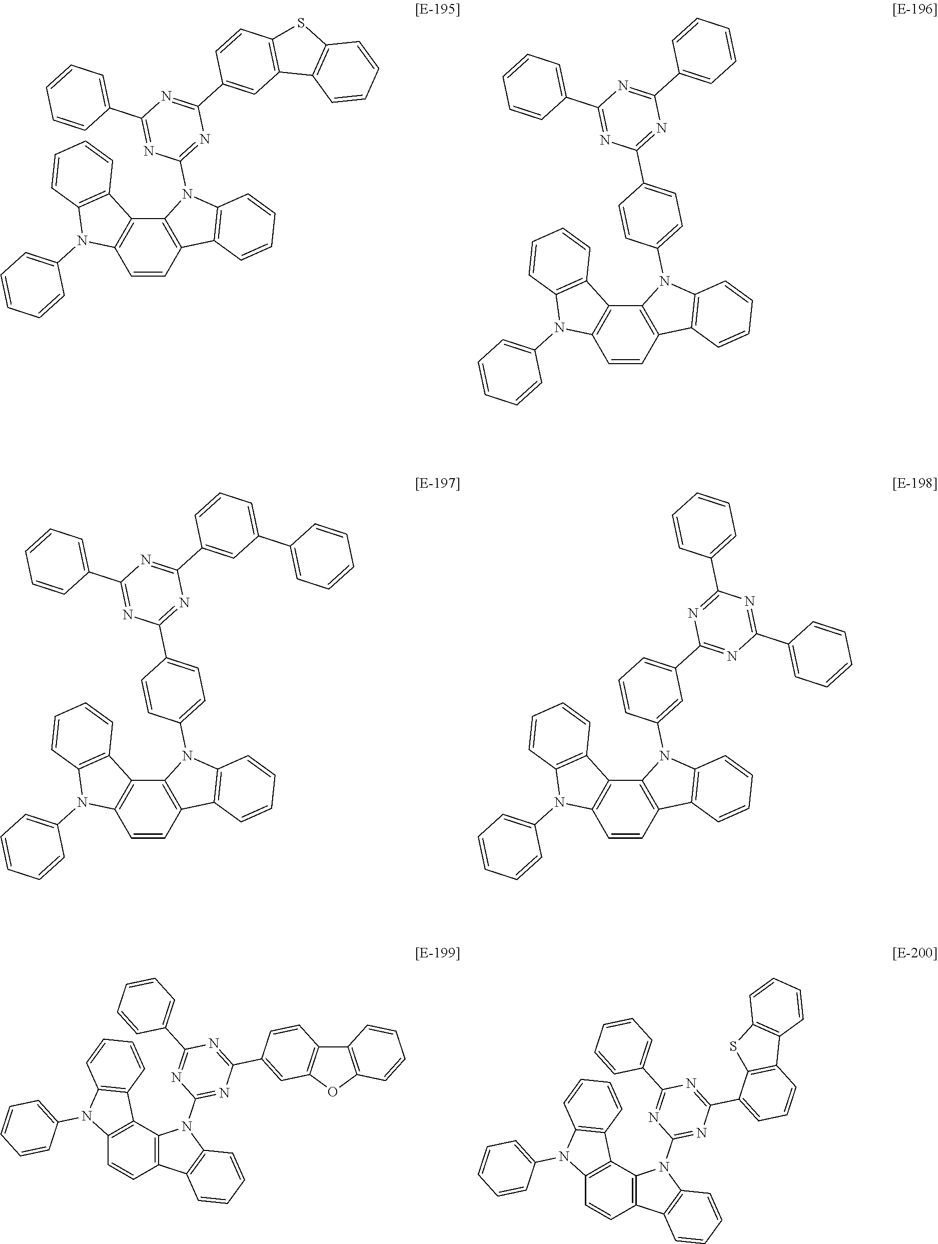

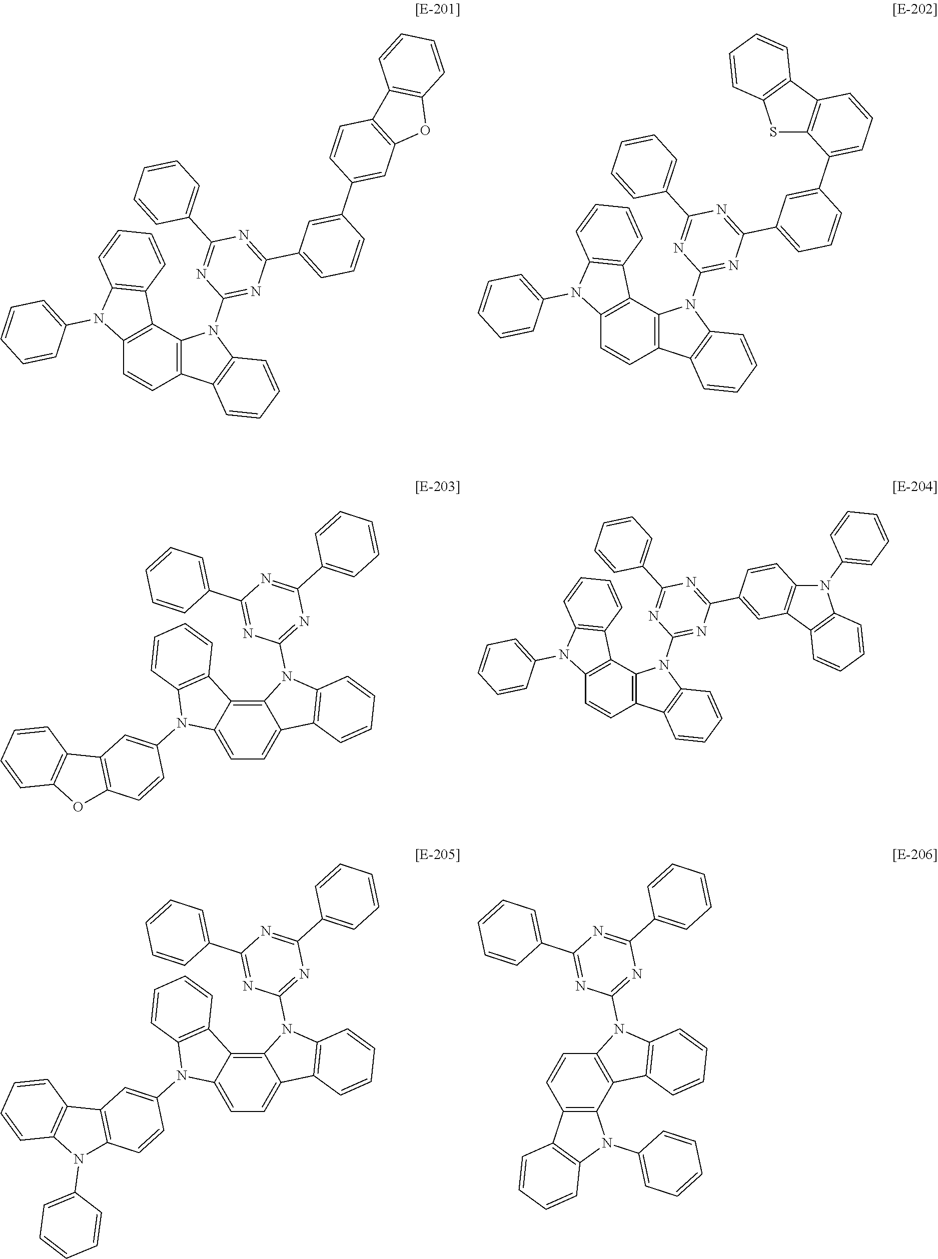

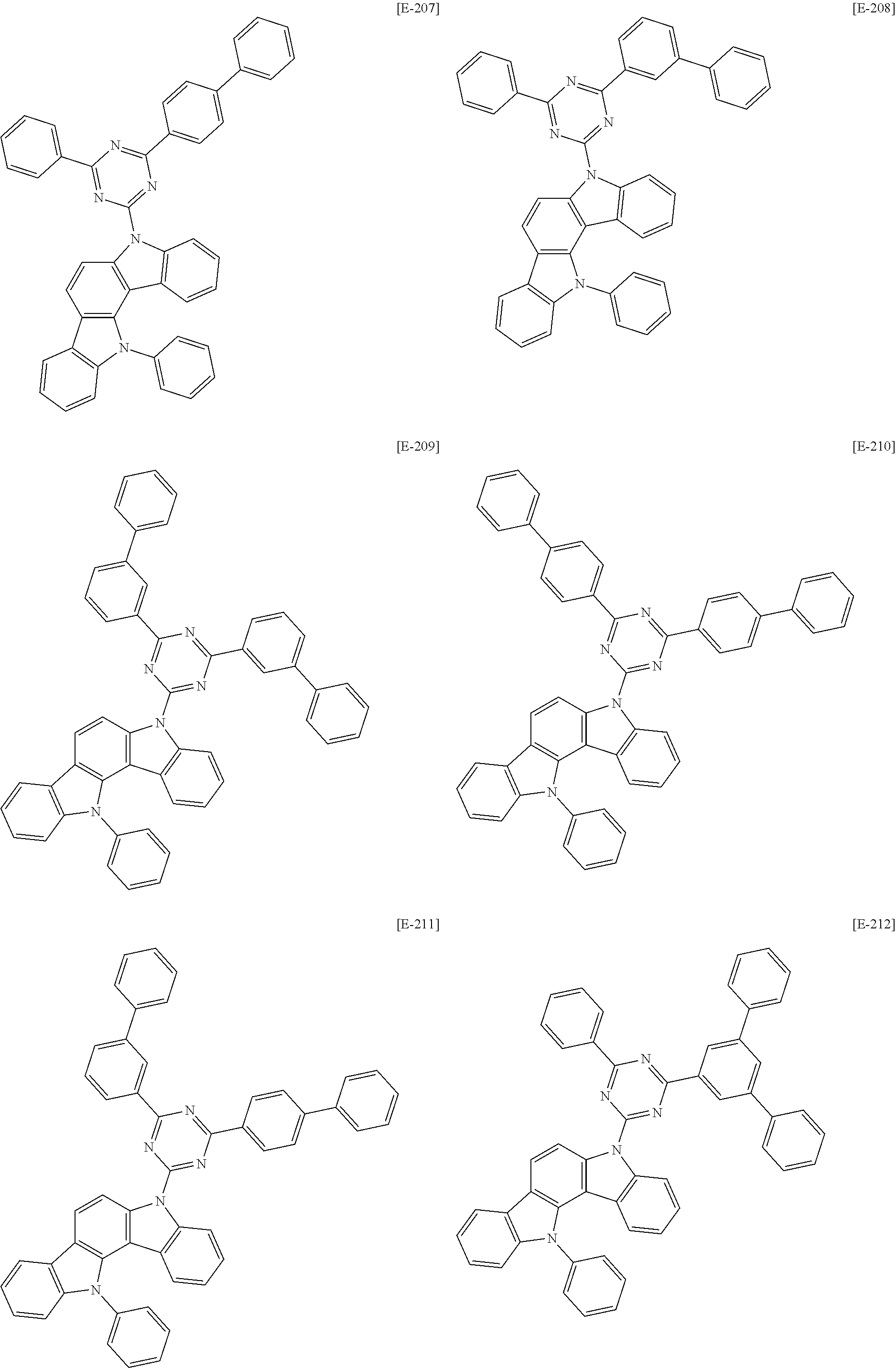

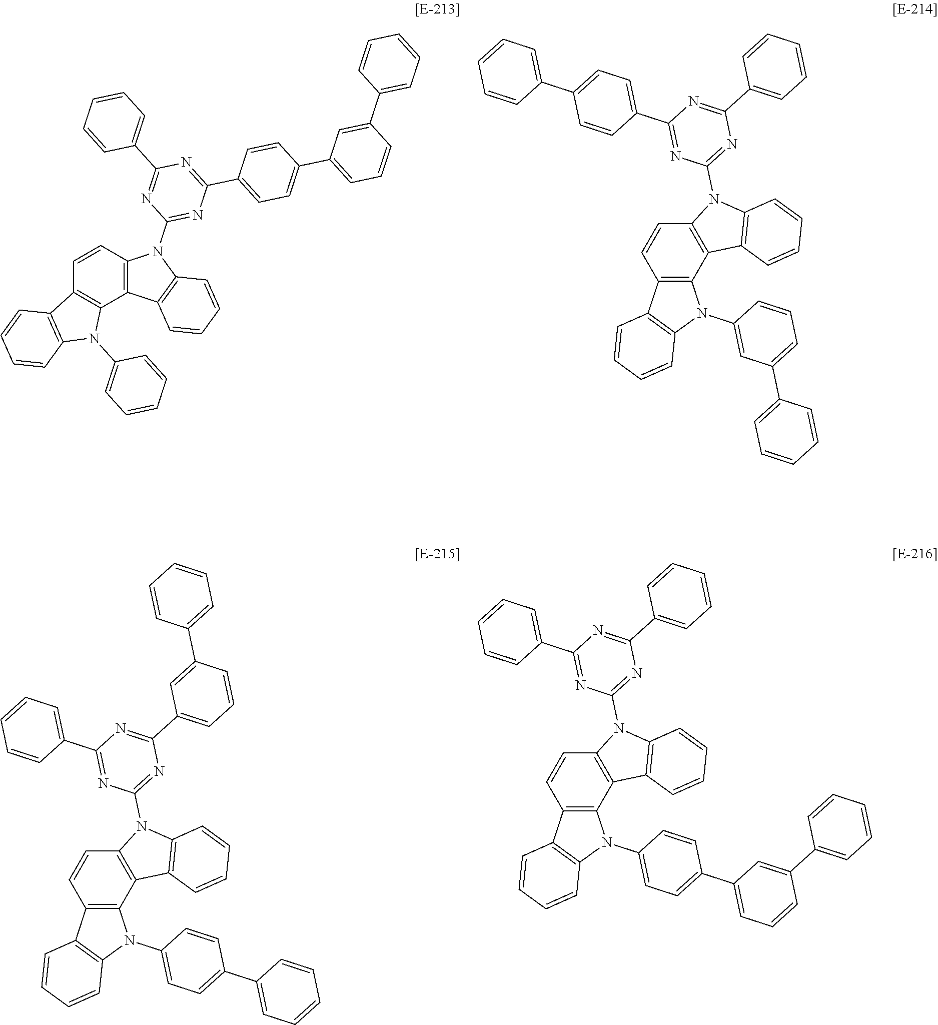 [SMILES: c1ccc(-c2cccc(-c3ccc(-c4nc(-c5ccccc5)nc(-n5c6ccccc6c6c5ccc5c7ccccc7n(-c7ccccc7)c56)n4)cc3)c2)cc1 ; c1ccc(-c2ccc(-c3nc(-c4ccccc4)nc(-n4c5ccccc5c5c4ccc4c6ccccc6n(-c6cccc(-c7ccccc7)c6)c45)n3)cc2)cc1 ; c1ccc(-c2ccc(-n3c4ccccc4c4ccc5c(c6ccccc6n5-c5nc(-c6ccccc6)nc(-c6cccc(-c7ccccc7)c6)n5)c43)cc2)cc1 ; c1ccc(-c2cccc(-c3ccc(-n4c5ccccc5c5ccc6c(c7ccccc7n6-c6nc(-c7ccccc7)nc(-c7ccccc7)n6)c54)cc3)c2)cc1]

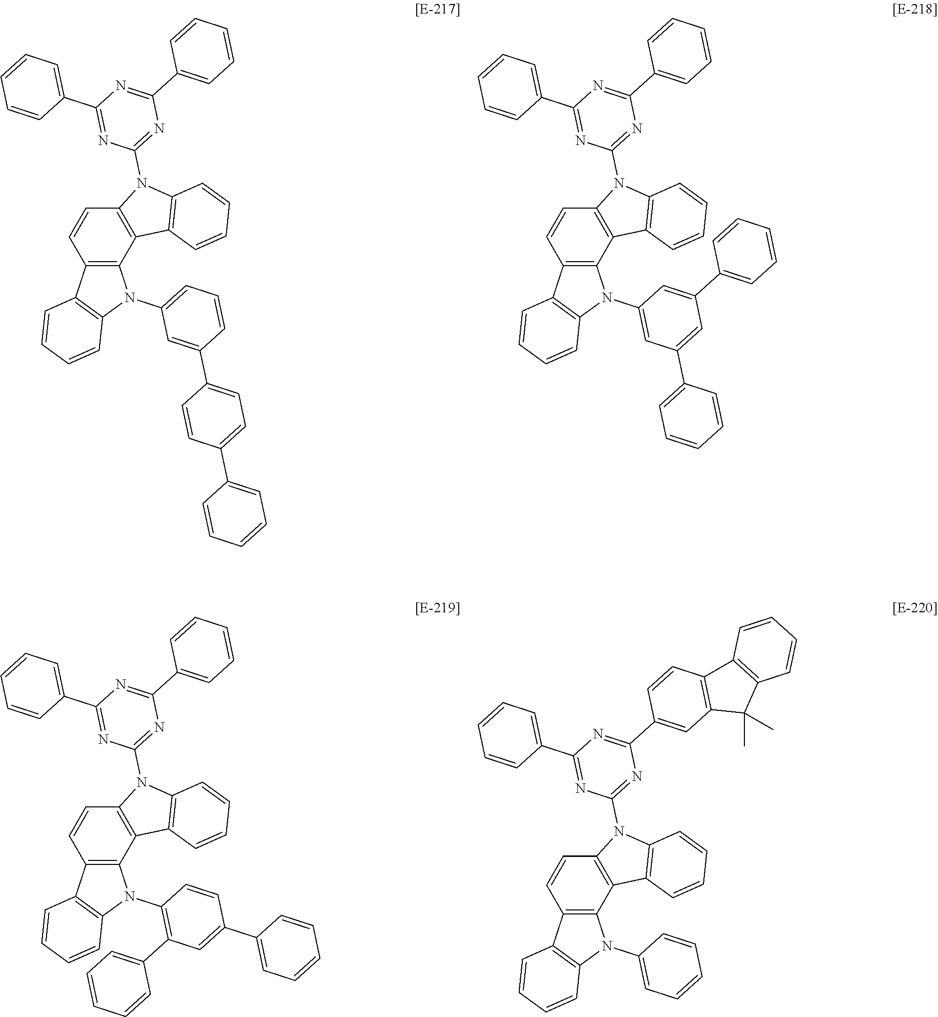 [SMILES: c1ccc(-c2ccc(-c3cccc(-n4c5ccccc5c5ccc6c(c7ccccc7n6-c6nc(-c7ccccc7)nc(-c7ccccc7)n6)c54)c3)cc2)cc1 ; c1ccc(-c2cc(-c3ccccc3)cc(-n3c4ccccc4c4ccc5c(c6ccccc6n5-c5nc(-c6ccccc6)nc(-c6ccccc6)n5)c43)c2)cc1 ; c1ccc(-c2ccc(-n3c4ccccc4c4ccc5c(c6ccccc6n5-c5nc(-c6ccccc6)nc(-c6ccccc6)n5)c43)c(-c3ccccc3)c2)cc1 ; CC1(C)c2ccccc2-c2ccc(-c3nc(-c4ccccc4)nc(-n4c5ccccc5c5c4ccc4c6ccccc6n(-c6ccccc6)c45)n3)cc21]

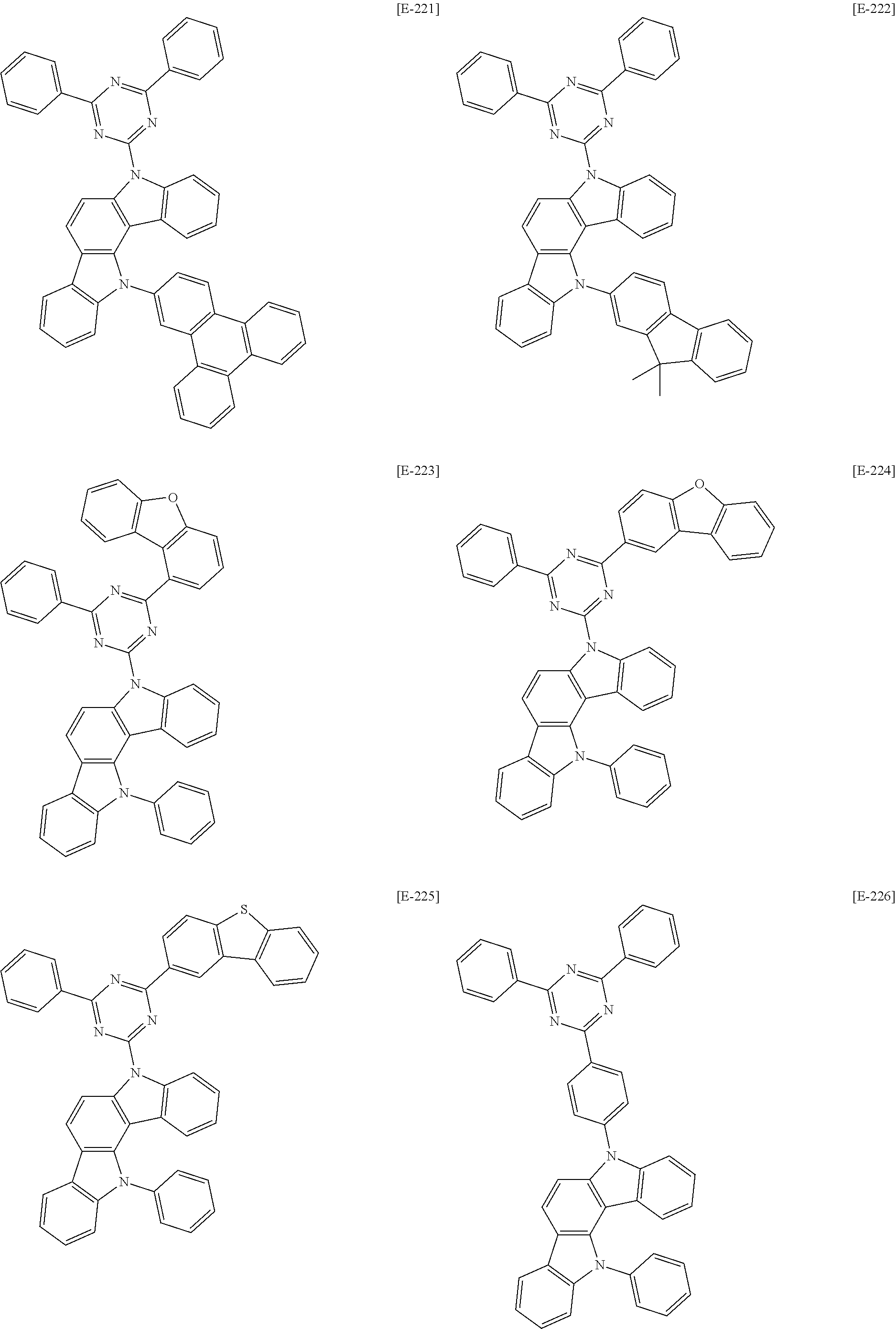

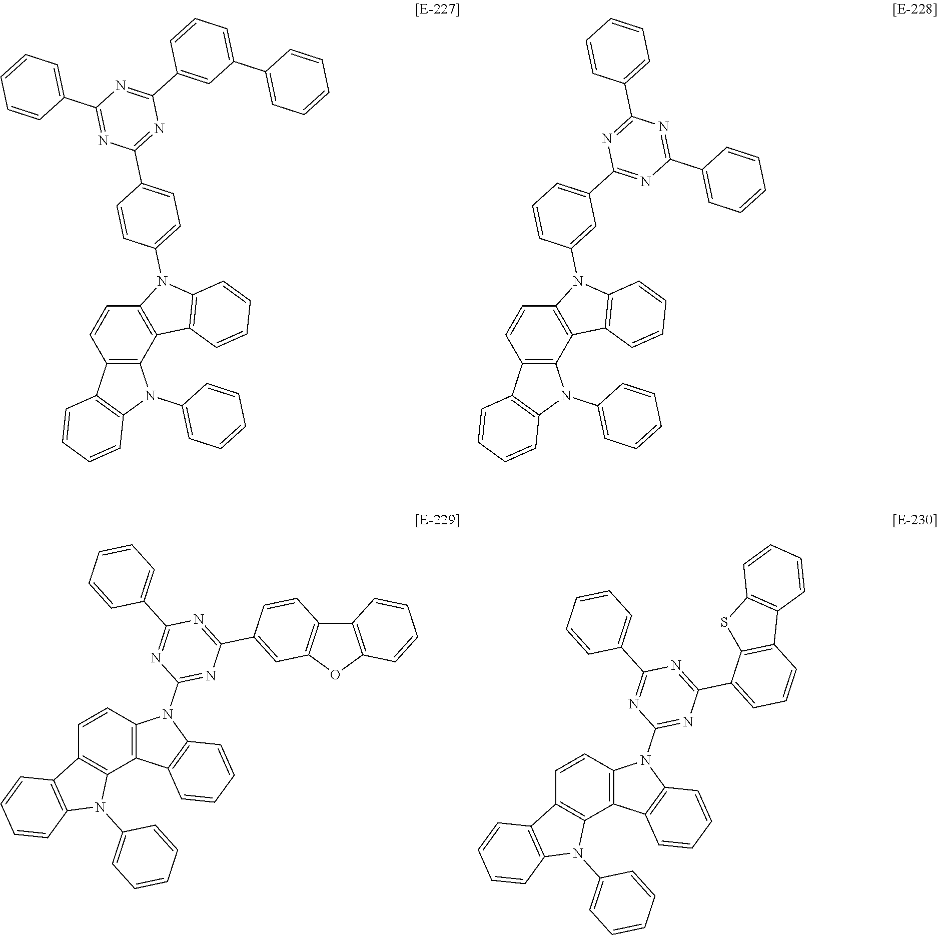 [SMILES: c1ccc(-c2cccc(-c3nc(-c4ccccc4)nc(-c4ccc(-n5c6ccccc6c6c5ccc5c7ccccc7n(-c7ccccc7)c56)cc4)n3)c2)cc1 ; c1ccc(-c2nc(-c3ccccc3)nc(-c3cccc(-n4c5ccccc5c5c4ccc4c6ccccc6n(-c6ccccc6)c45)c3)n2)cc1 ; c1ccc(-c2nc(-c3ccc4c(c3)oc3ccccc34)nc(-n3c4ccccc4c4c3ccc3c5ccccc5n(-c5ccccc5)c34)n2)cc1 ; c1ccc(-c2nc(-c3cccc4c3sc3ccccc34)nc(-n3c4ccccc4c4c3ccc3c5ccccc5n(-c5ccccc5)c34)n2)cc1]

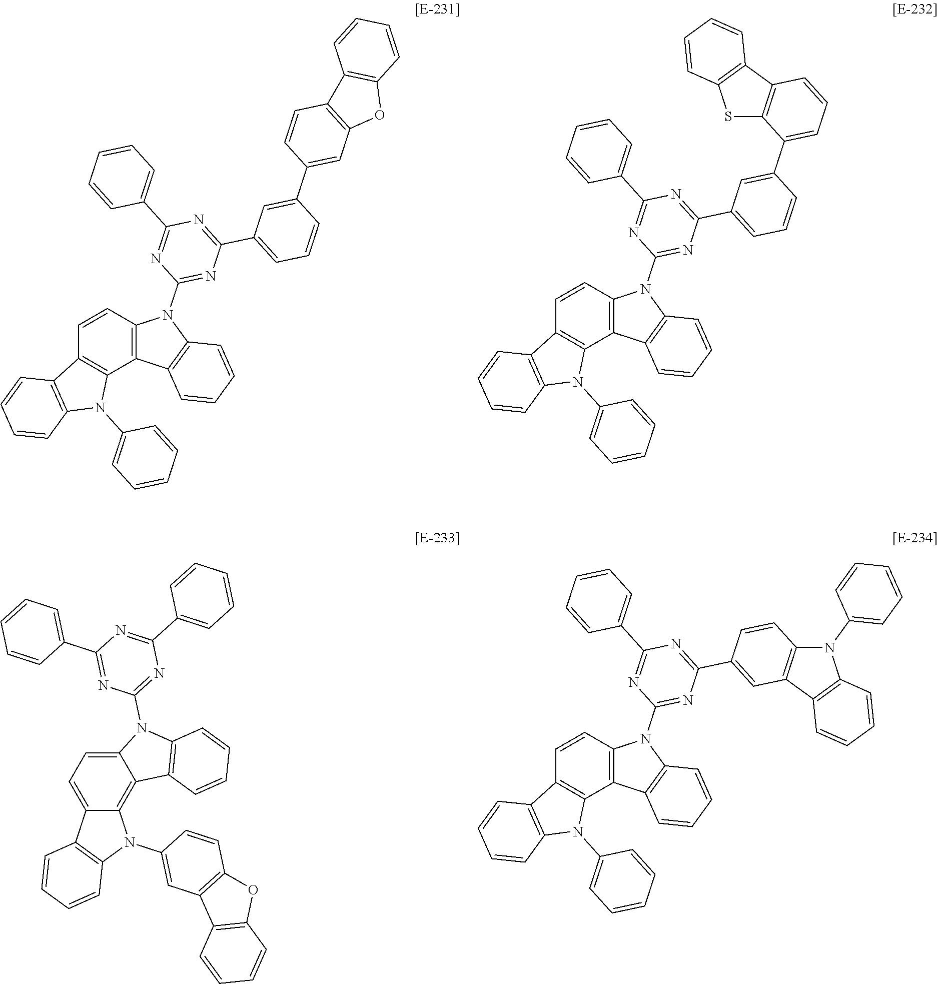 [SMILES: c1ccc(-c2nc(-c3cccc(-c4ccc5c(c4)oc4ccccc45)c3)nc(-n3c4ccccc4c4c3ccc3c5ccccc5n(-c5ccccc5)c34)n2)cc1 ; c1ccc(-c2nc(-c3cccc(-c4cccc5c4sc4ccccc45)c3)nc(-n3c4ccccc4c4c3ccc3c5ccccc5n(-c5ccccc5)c34)n2)cc1 ; c1ccc(-c2nc(-c3ccccc3)nc(-n3c4ccccc4c4c3ccc3c5ccccc5n(-c5ccc6oc7ccccc7c6c5)c34)n2)cc1 ; c1ccc(-c2nc(-c3ccc4c(c3)c3ccccc3n4-c3ccccc3)nc(-n3c4ccccc4c4c3ccc3c5ccccc5n(-c5ccccc5)c34)n2)cc1]

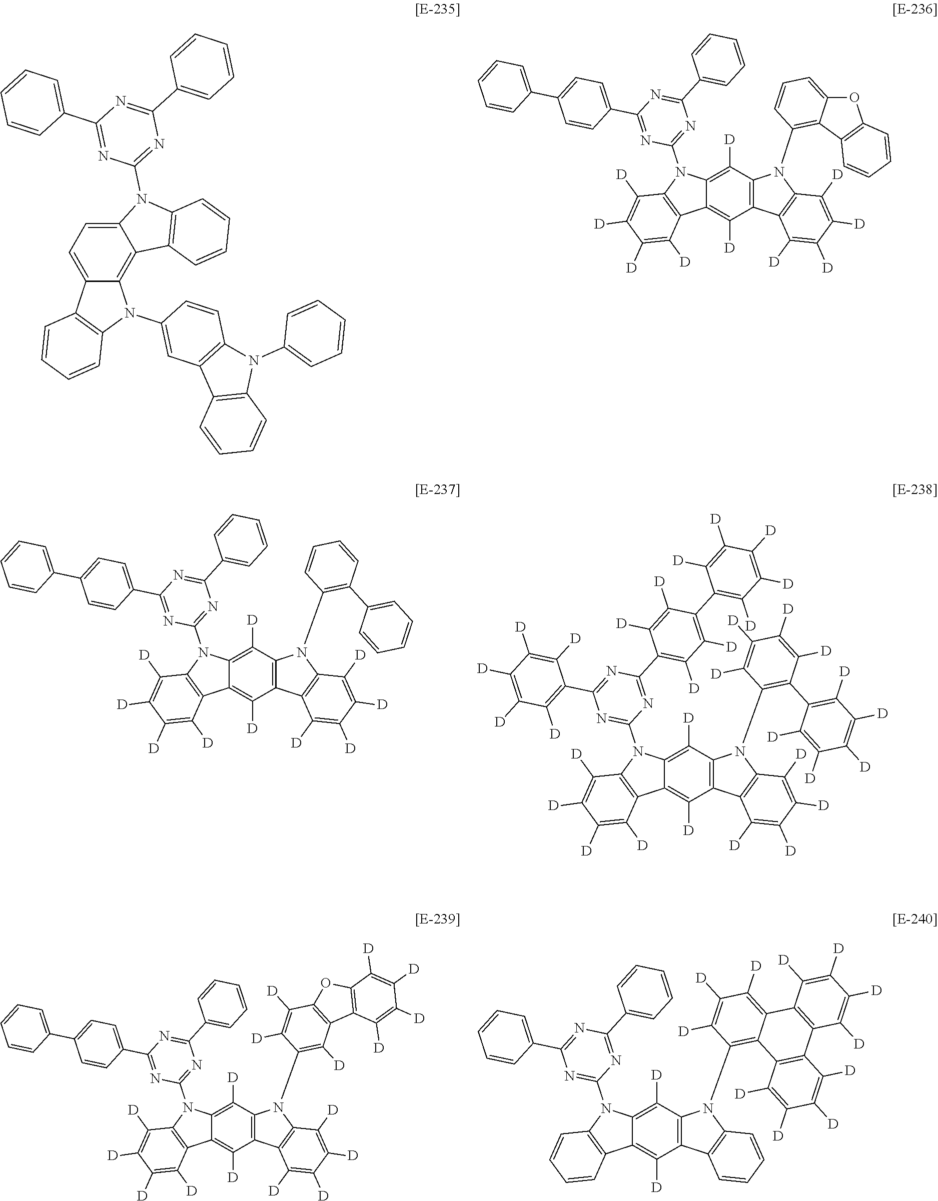

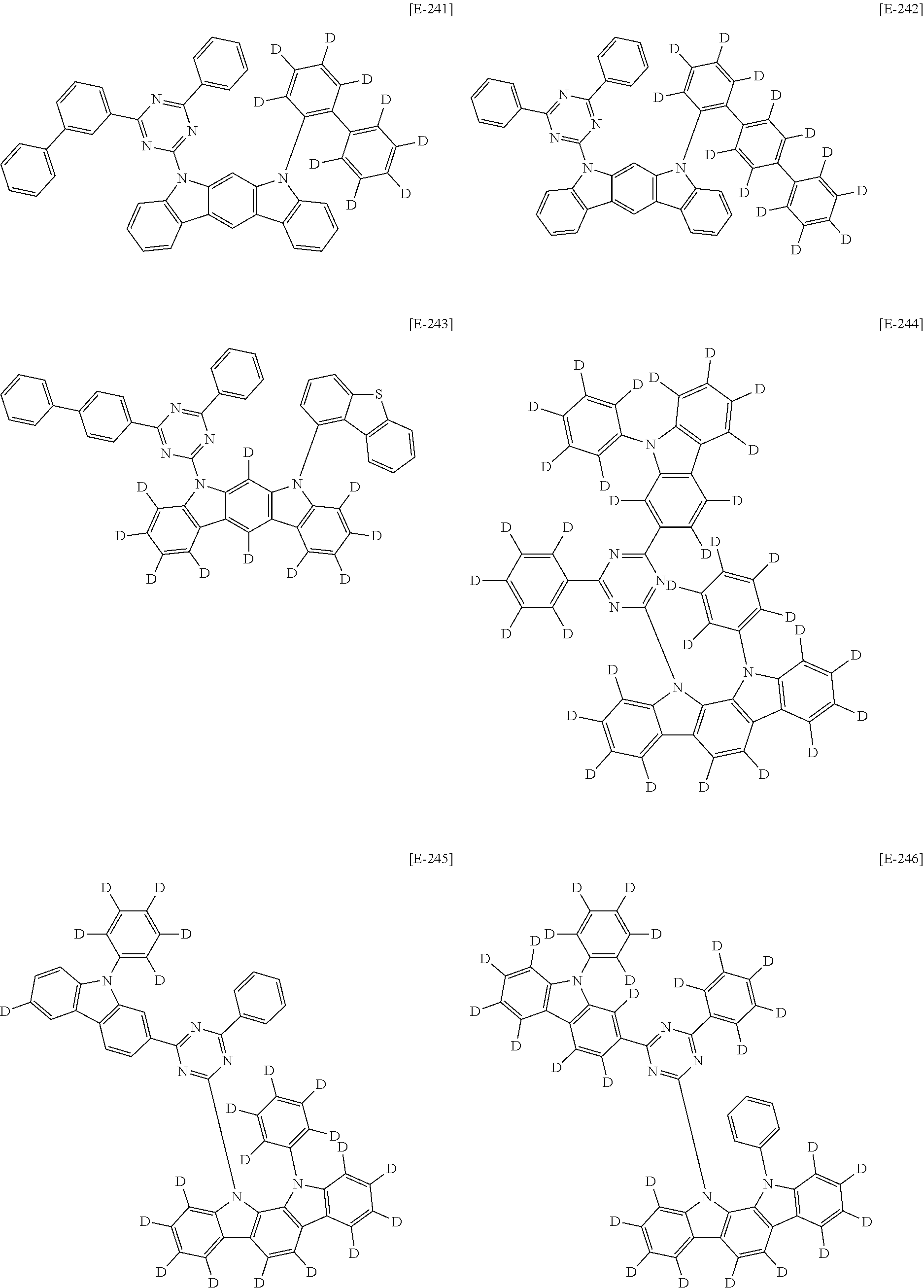

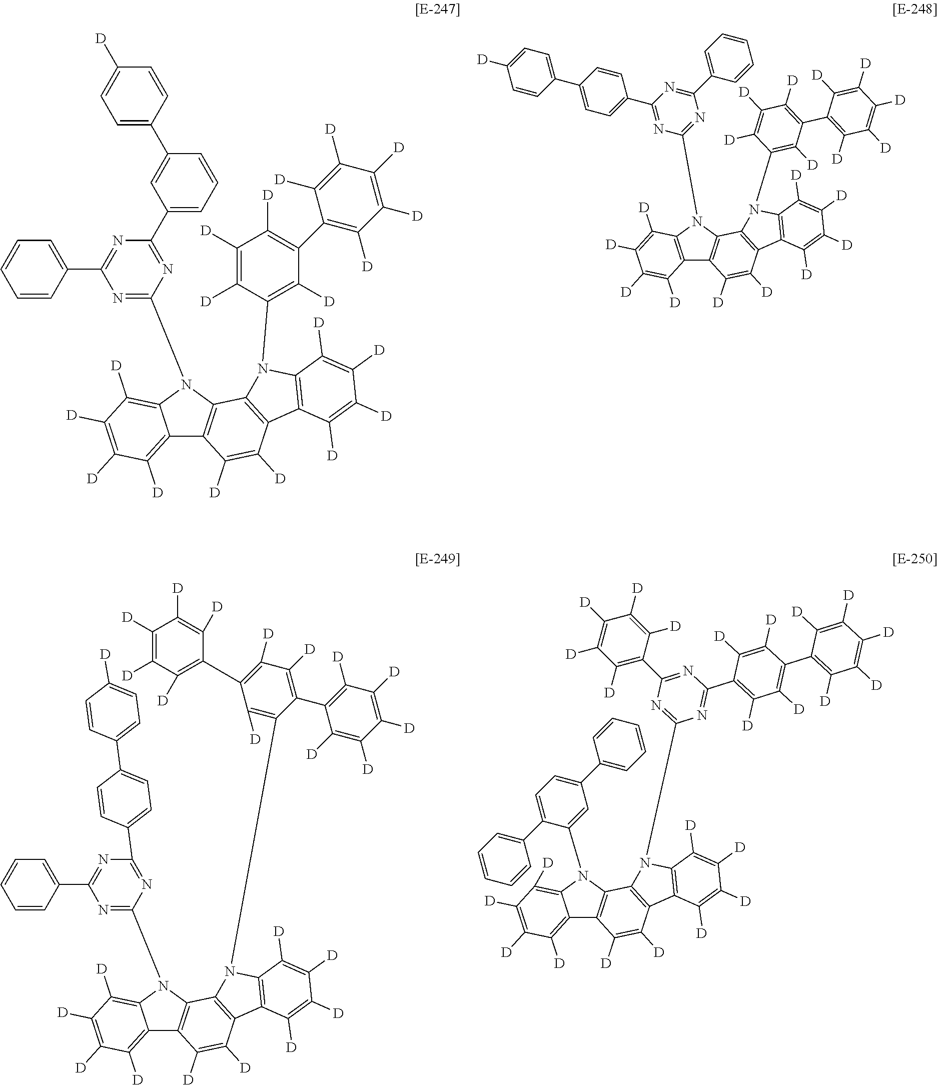

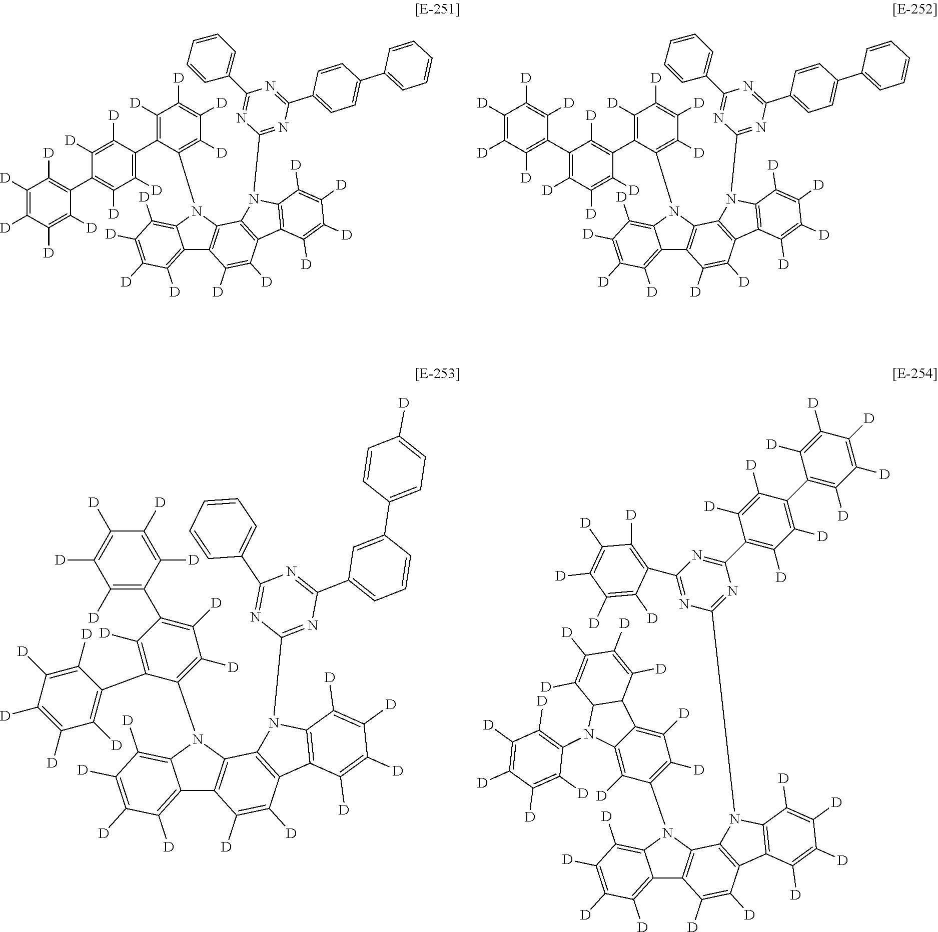

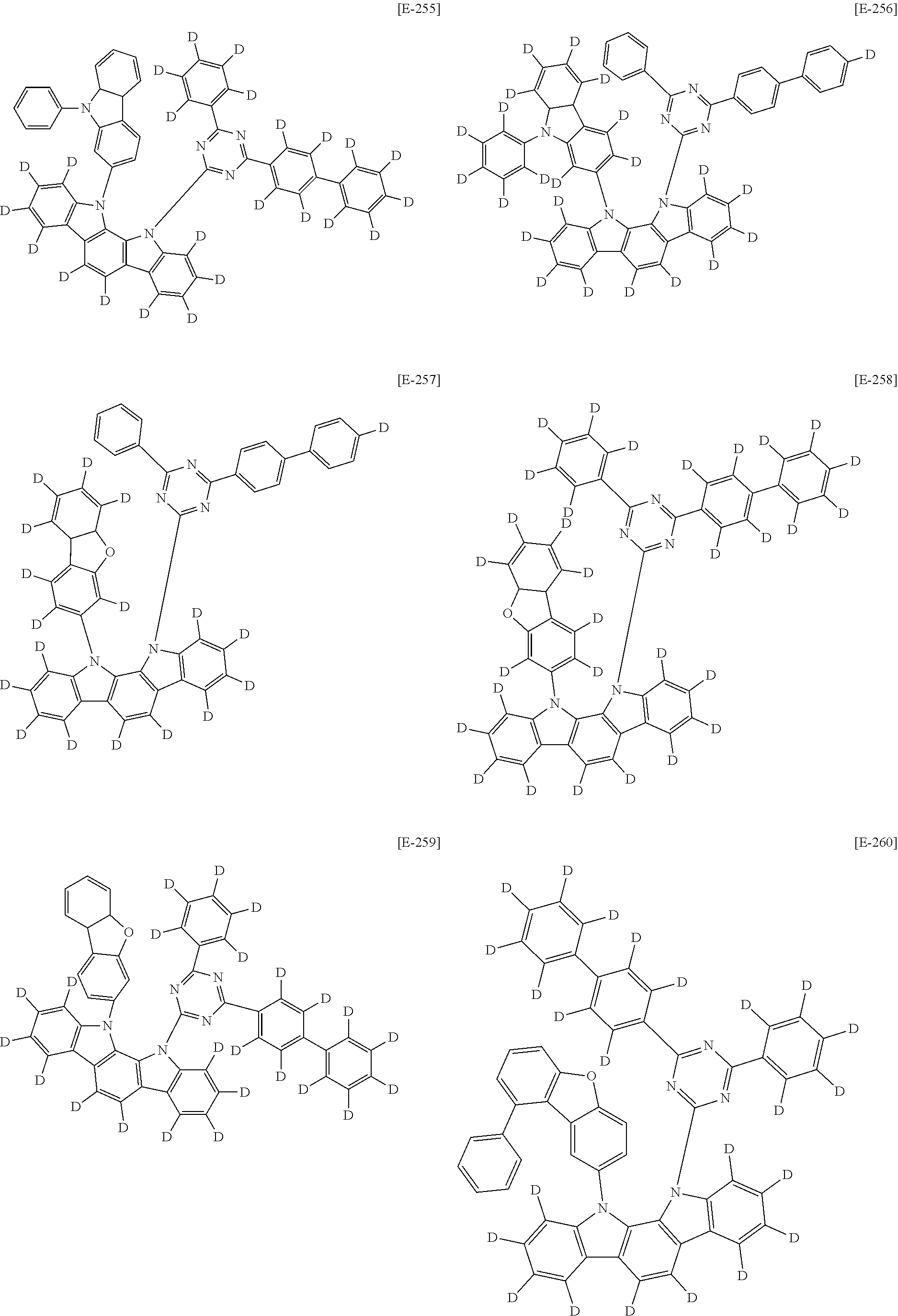

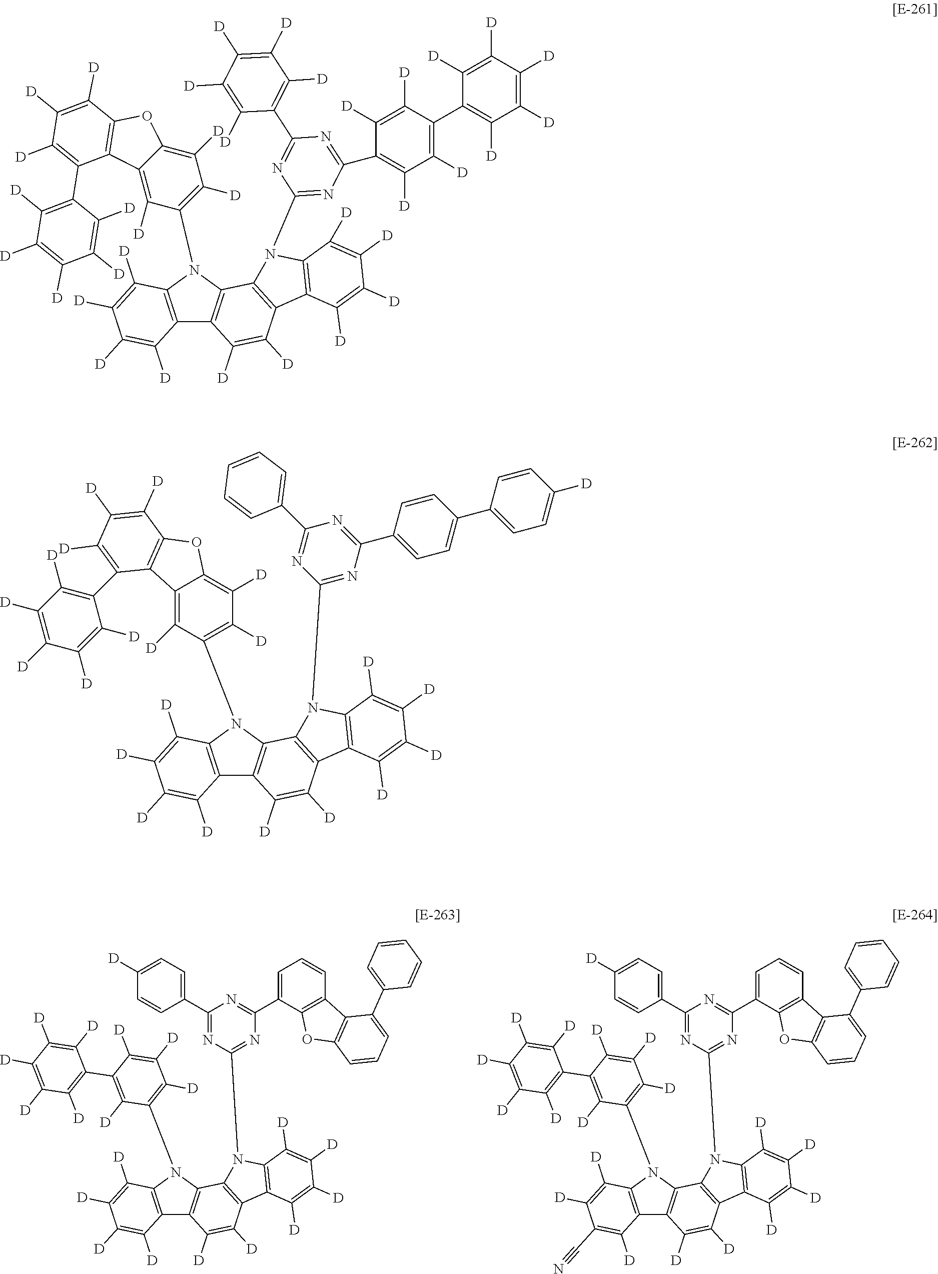

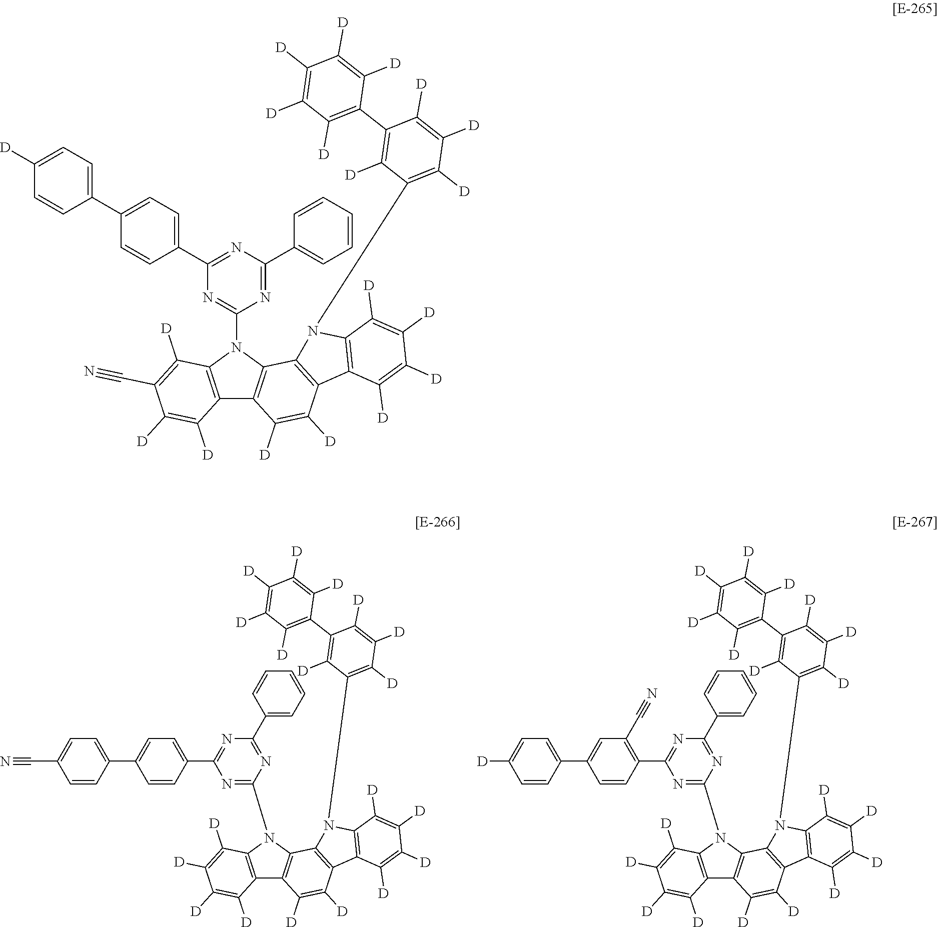

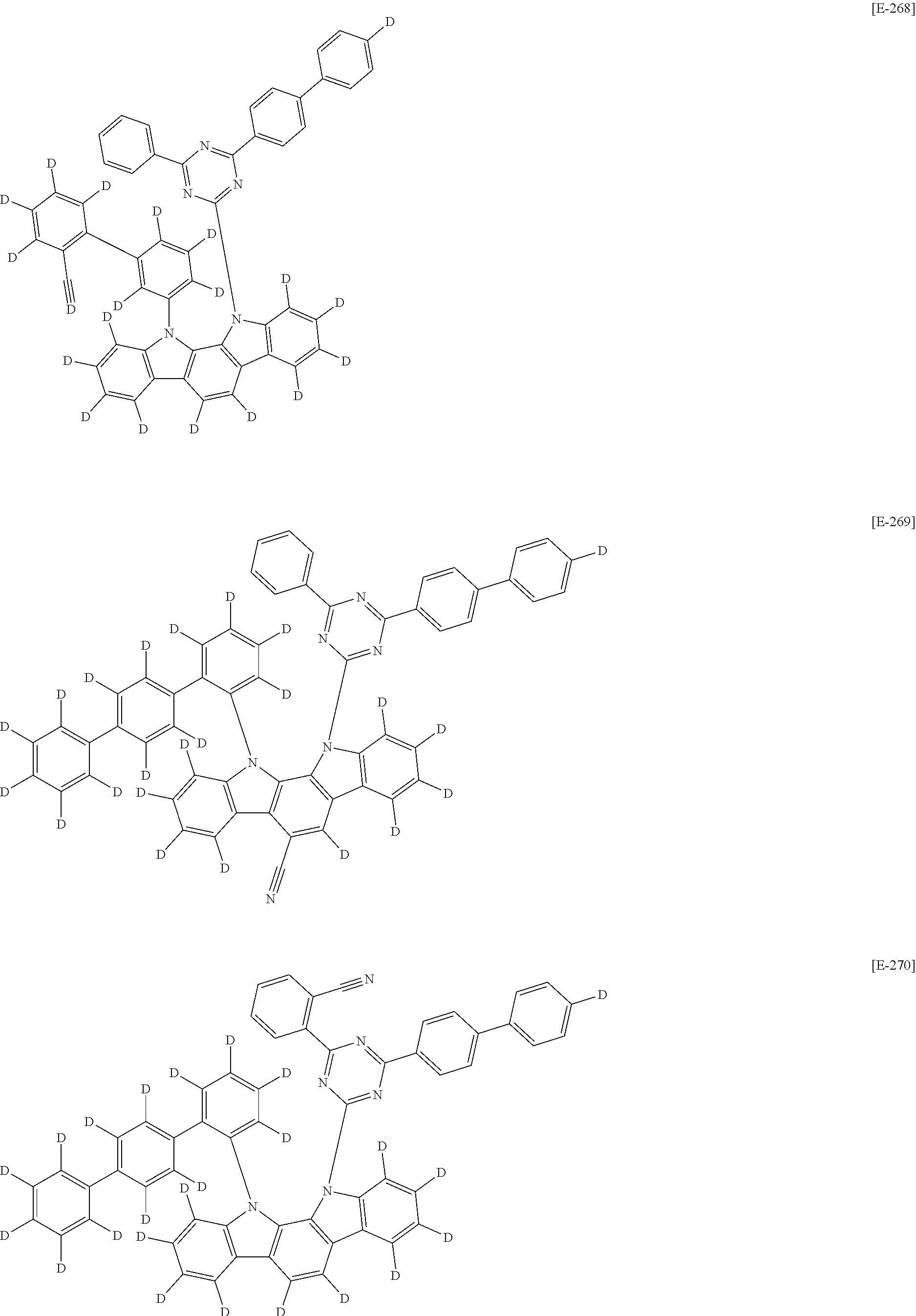

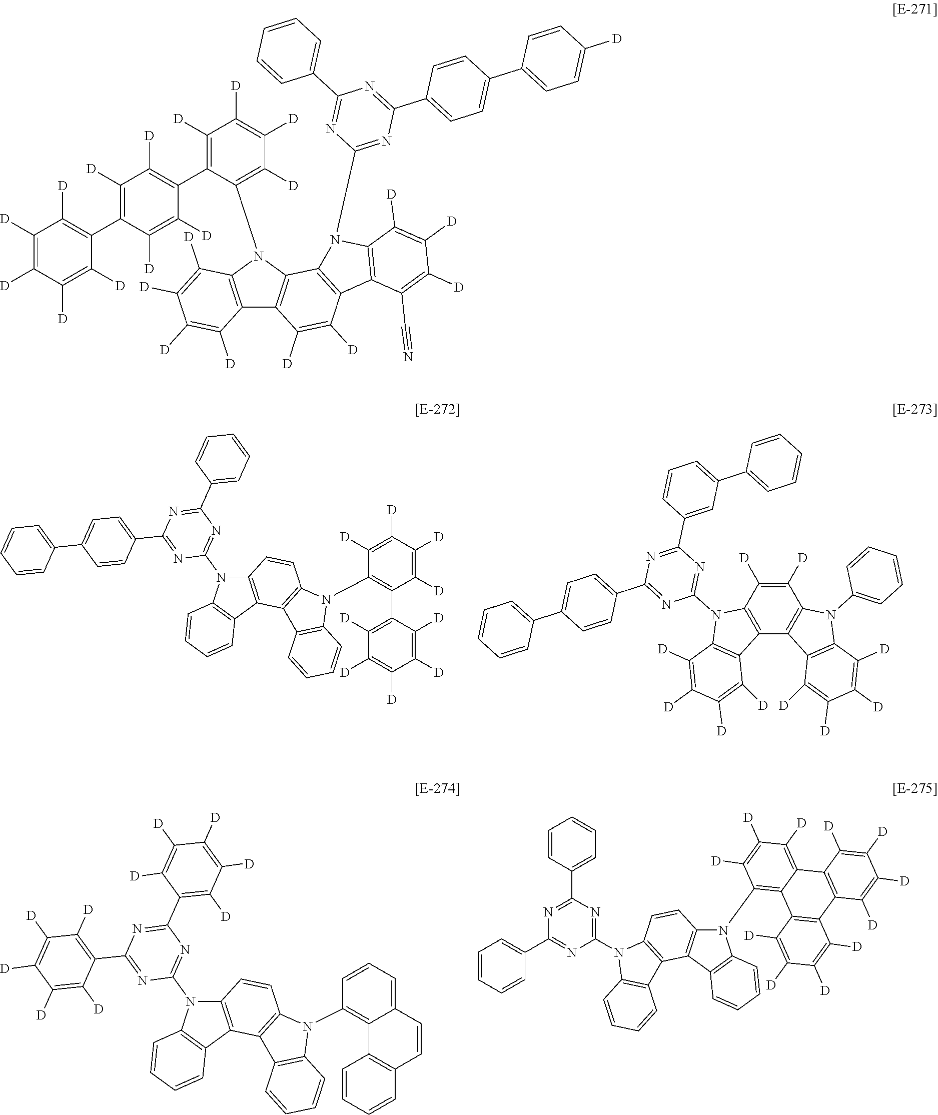

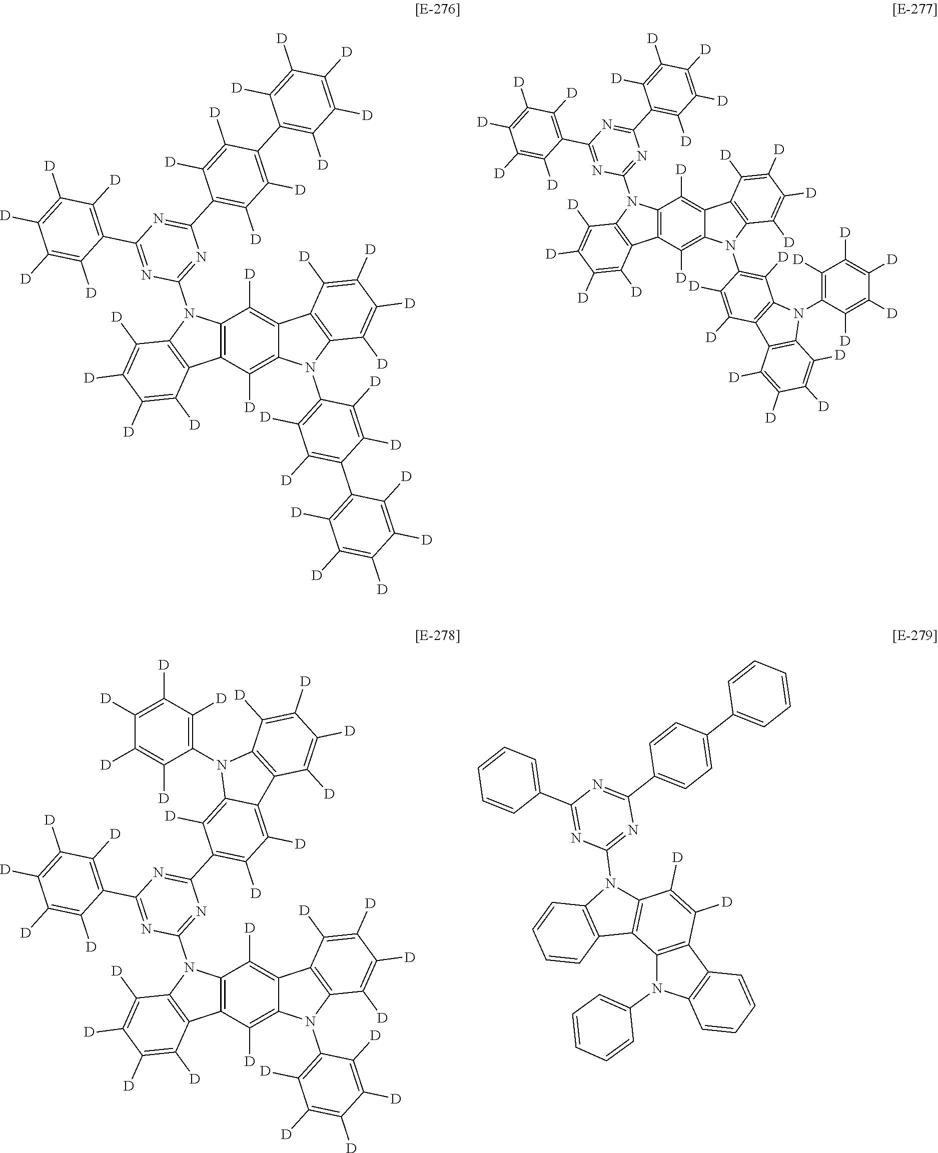

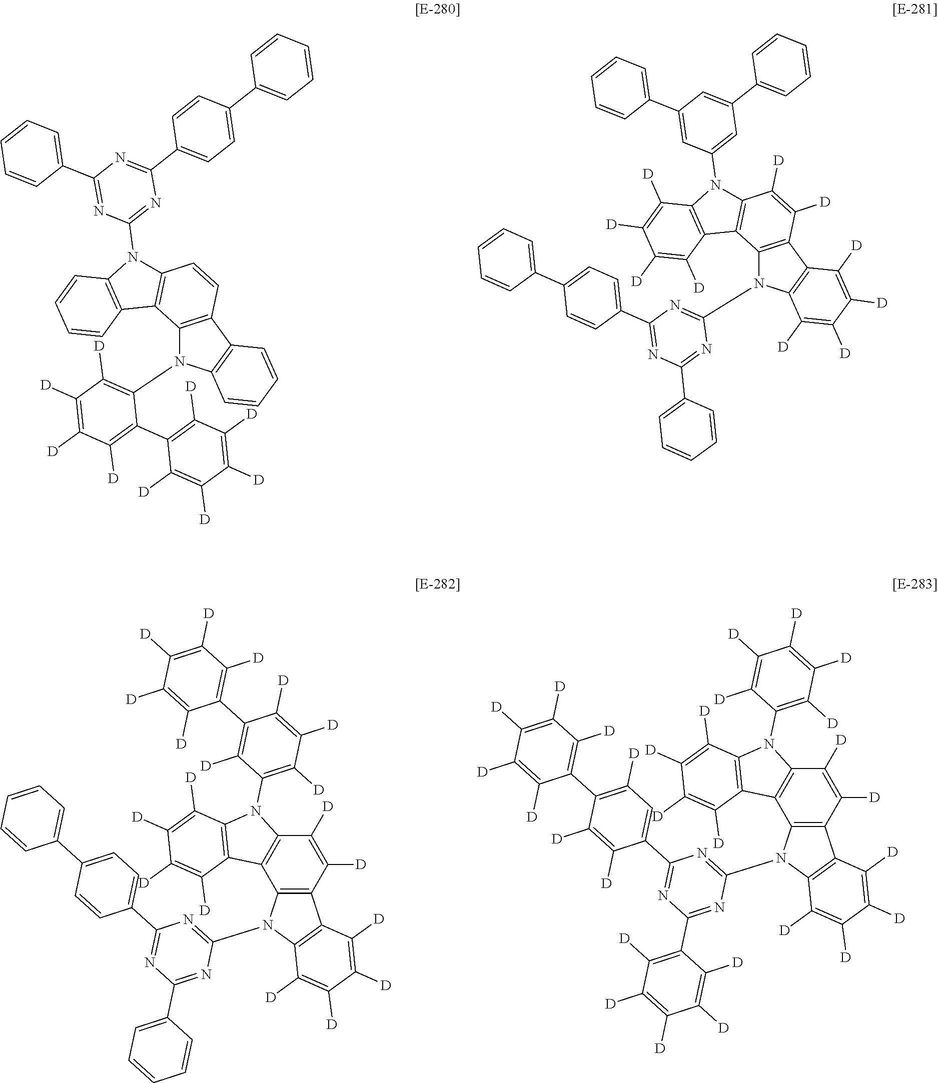

The first compound and the second compound may be included or mixed in a weight ratio of, e.g., 1:99 to 99:1. By being included in the above range, efficiency and life-span may be improved by implementing bipolar characteristics by adjusting the appropriate weight ratio using the hole transport capability of the first compound and the electron transport capability of the second compound. In an implementation, they may be included in a weight ratio of, e.g., about 10:90 to 90:10, about 20:80 to 80:20, for example, about 20:80 to about 70:30, about 20:80 to about 60:40, or about 30:70 to about 60:40. In an implementation, they may be included in a weight ratio of 40:60, 50:50, or 60:40.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

The FIGURE is a cross-sectional view showing an organic light emitting diode according to some example embodiments.

Referring to the FIGURE, an organic light emitting diode 100 according to some example embodiments may include an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, or the like or an alloy thereof, a metal oxide such as zinc oxide, indium oxide, indium tin oxide (ITO), indium zinc oxide (IZO), or the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, and/or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof, or a multi-layer structure material such as LiF/Al, LiO₂/Al, LiF/Ca, or BaF₂/Ca.

The organic layer 105 may include the aforementioned compound for an organic optoelectronic device or composition for an organic optoelectronic device.

The organic layer 105 may include a light emitting layer 130, the light emitting layer 130 may include a host and a dopant, the host may include the aforementioned compound for an organic optoelectronic device or the composition for an organic optoelectronic device, and the dopant may be, e.g., a phosphorescent dopant, such as a red, green, or blue phosphorescent dopant, e.g., a red or green phosphorescent dopant.

The dopant may be a material mixed with the compound or composition for an organic optoelectronic device in a small amount to cause light emission and may be a material such as a metal complex that emits light by multiple excitation into a triplet or more. The dopant may be, e.g., an inorganic, organic, or organic-inorganic compound, and one or more types thereof may be used.

Examples of the dopant may include a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. In an implementation, the phosphorescent dopant may be, e.g., a compound represented by Chemical Formula Z.

L⁵MX³ [Chemical Formula Z]

In Chemical Formula Z, M may be a metal, and L⁵ and X³ may each independently be, e.g., a ligand to form a complex compound with M.

The M may be, e.g., Ir, Pt, Os, Ti, Zr, Hf, Eu, Tb, Tm, Fe, Co, Ni, Ru, Rh, Pd, or a combination thereof, and L⁵ and X³ may each independently be, e.g., a bidentate ligand.

Examples of the ligands represented by L⁵ and X³ may include a ligand of Group A.

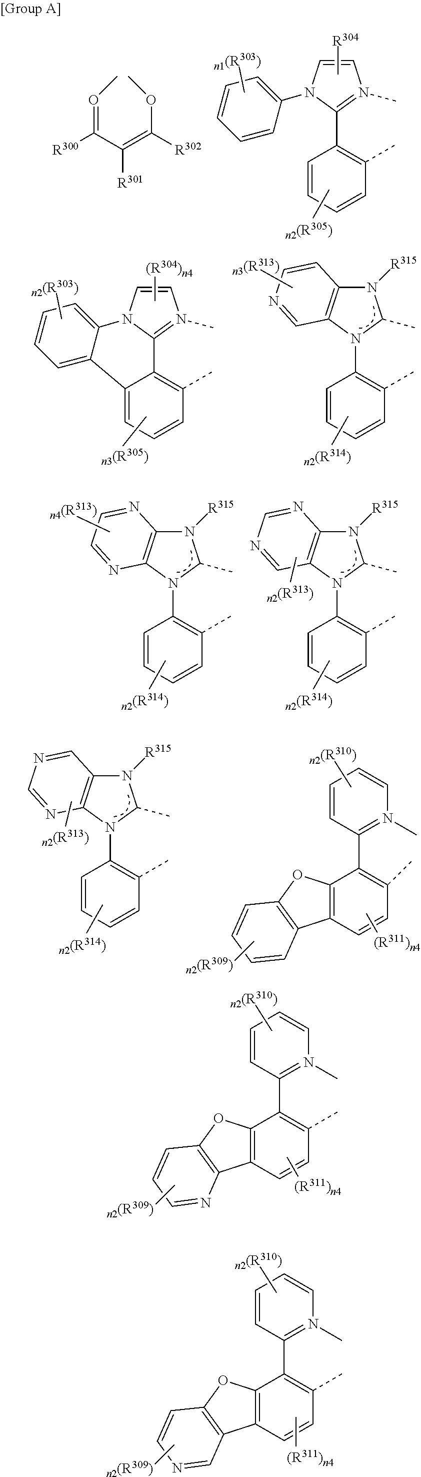

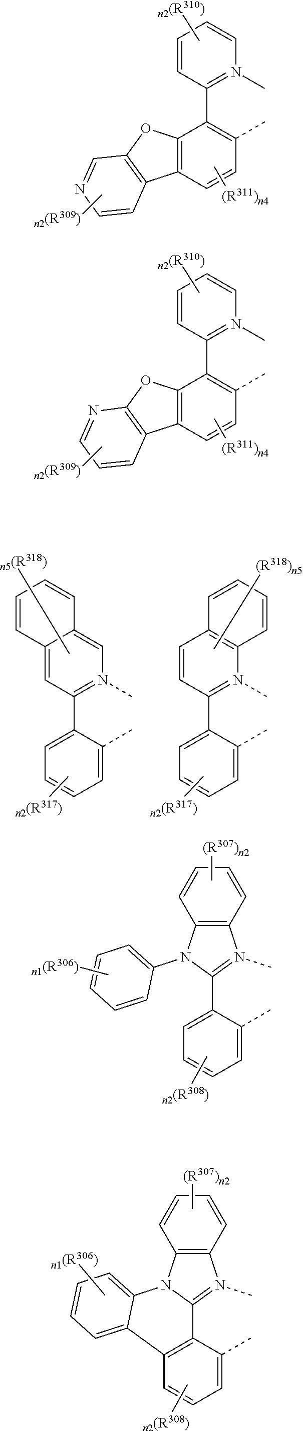

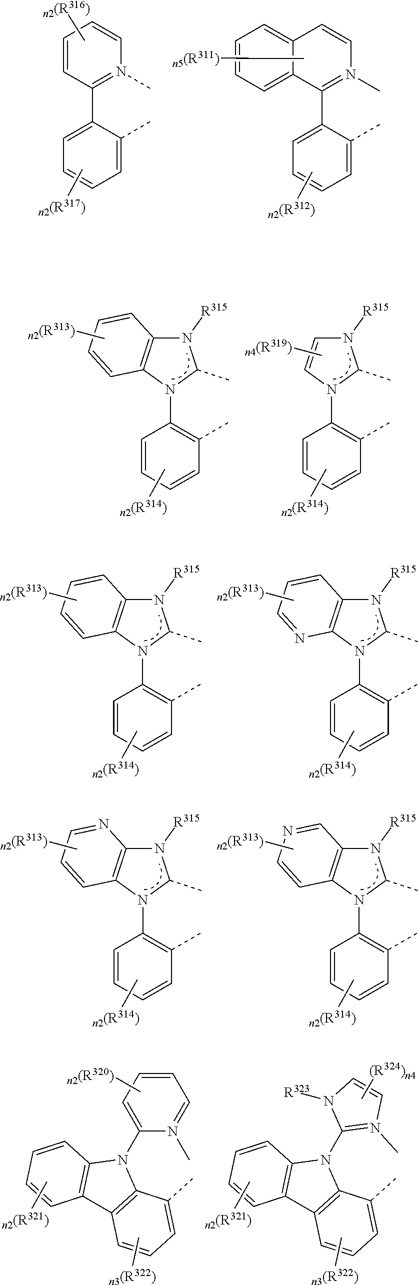

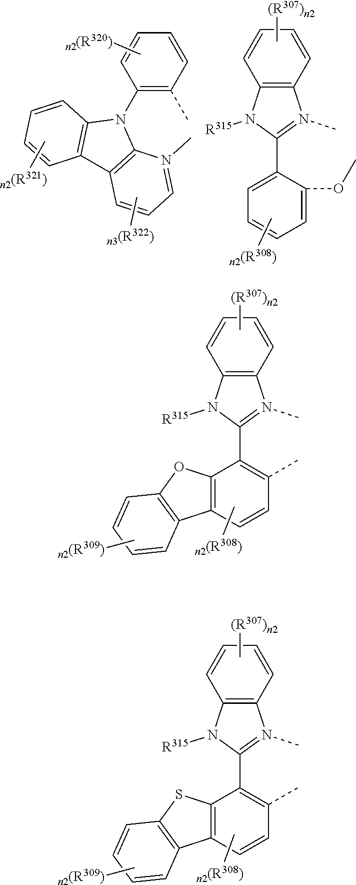

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰³ to R³²⁴ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

The dopant according to some example embodiments may be an iridium complex, and may include, e.g., a dopant represented by Chemical Formula 4-1 or Chemical Formula 4-2.

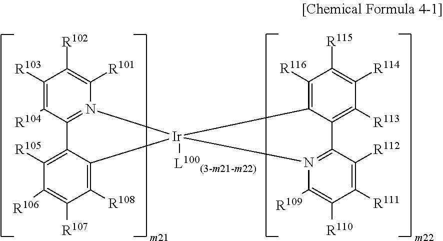

In Chemical Formula 4-1, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹⁰¹ to R¹¹⁶ may be a functional group represented by Chemical Formula V-1.

L¹⁰⁰ may be, e.g., a bidentate ligand of a monovalent anion, and is a ligand that coordinates to iridium through a lone pair of carbons or heteroatoms.

m21 and m22 may each independently be, e.g., an integer from 0 to 3, and m21+m22 may be integer from 1 to 3.

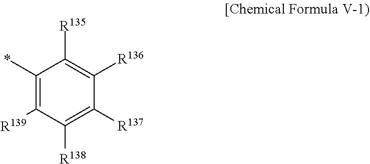

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* refers to the portion linked to the carbon atom.

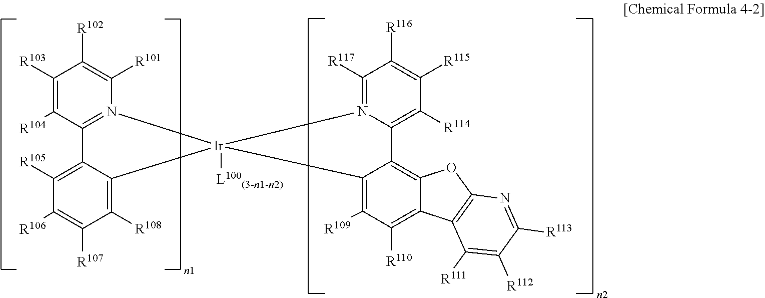

In Chemical Formula 4-2, R¹⁰¹ to R¹¹⁷ may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

L¹⁰⁰ may be, e.g., a bidentate ligand of a monovalent anion, and is a ligand that coordinates to iridium through a lone pair of carbons or heteroatoms.

n1 and n2 may each independently be, e.g., an integer of 0 to 3, and n1+n2 may be an integer of 1 to 3.

The dopant according to some example embodiments may be a platinum complex, and may be represented, e.g., by Chemical Formula Z-1.

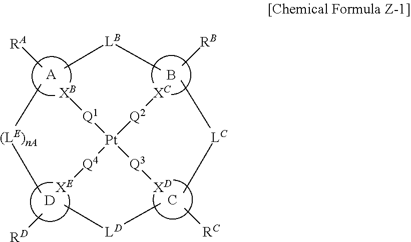

In Chemical Formula Z-1, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution;

L^{B}, L_{C}, and L^{D} may each independently be, e.g., a direct bond, BR, NR, PR, O, S, Se, C═O, S═O, SO₂, CRR′, SiRR′, GeRR′, or a combination thereof.

In an implementation, nA may be 1, and L^{E} may be a direct bond, BR, NR, PR, O, S, Se, C═O, S═O, SO₂, CRR′, SiRR′, GeRR′, or a combination thereof. In an implementation, nA may be 0, and L^{E} does not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ are optionally linked to each other to provide a ring; X^{B}, X^{c}, X^{D}, and X^{E} are each independently selected from carbon and nitrogen; and Q¹, Q², Q³, and Q⁴ each represent oxygen or a direct bond.

The platinum complex may be represented, e.g., by Chemical Formula 5-1 or Chemical Formula 5-2.

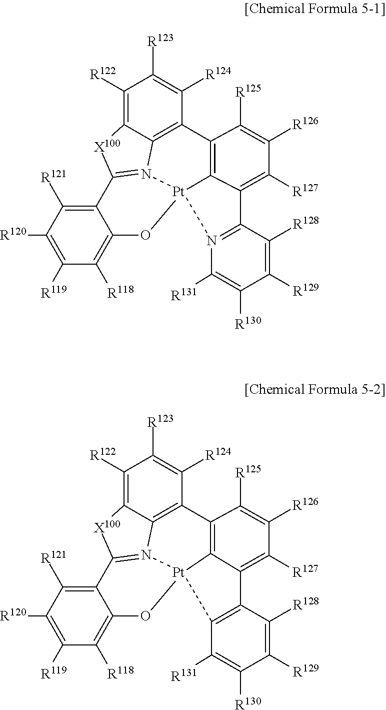

In Chemical Formula 5-1 and Chemical Formula 5-2, X¹⁰⁰ may be, e.g., O, S, or N¹³².

R¹¹⁸ to R¹³² may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹¹⁹ to R¹³² may be —SiR¹³³R¹³⁴R¹³⁵ or a tert-butyl group.

R¹³³ to R¹³⁵ may each independently be, e.g., a substituted or unsubstituted C1 to C6 alkyl group.

The organic layer may further include a charge transport region in addition to the light emitting layer.

The charge transport region may be, e.g., a hole transport region 140.

The hole transport region 140 may help further increase hole injection and/or hole mobility between the anode 120 and the light emitting layer 130 and block electrons.

In an implementation, the hole transport region 140 may include a hole transport layer between the anode 120 and the light emitting layer 130, and a hole transport auxiliary layer between the light emitting layer 130 and the hole transport layer, and a compound of Group B may be included in at least one of the hole transport layer and the hole transport auxiliary layer.

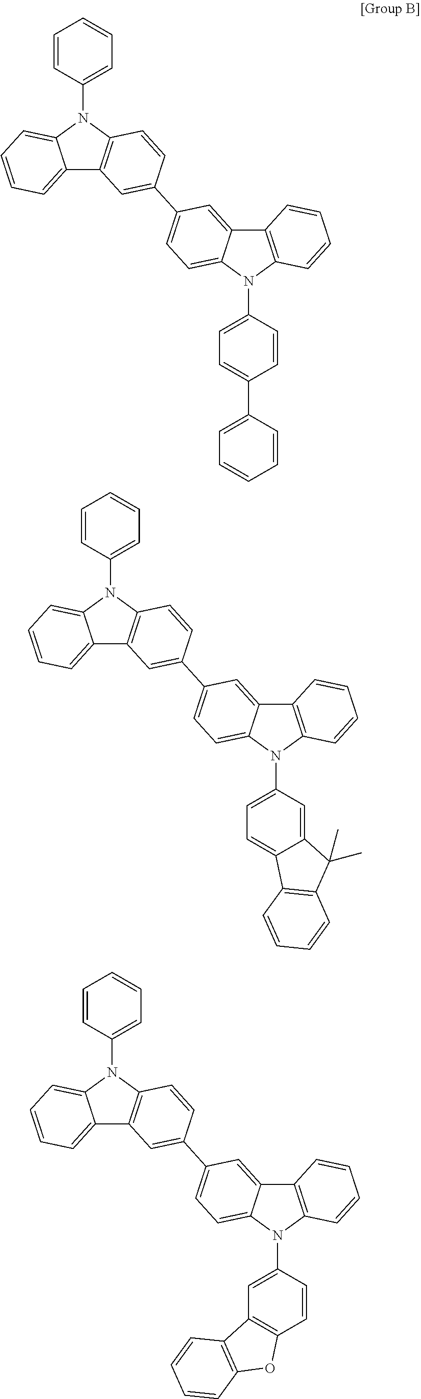 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

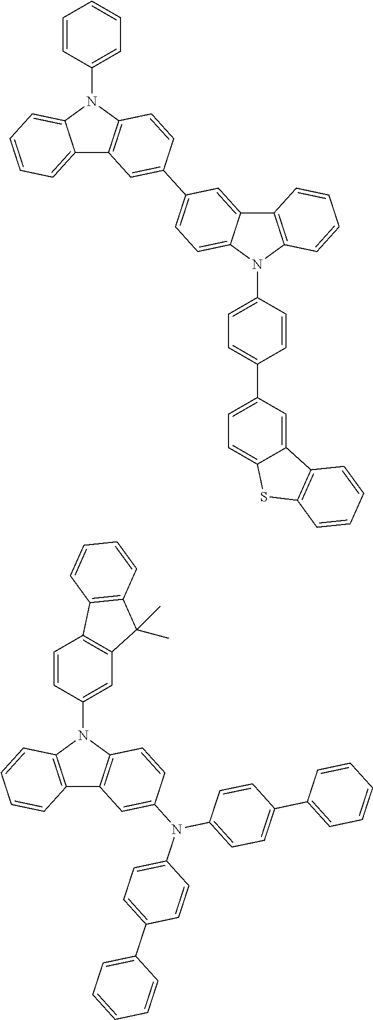 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

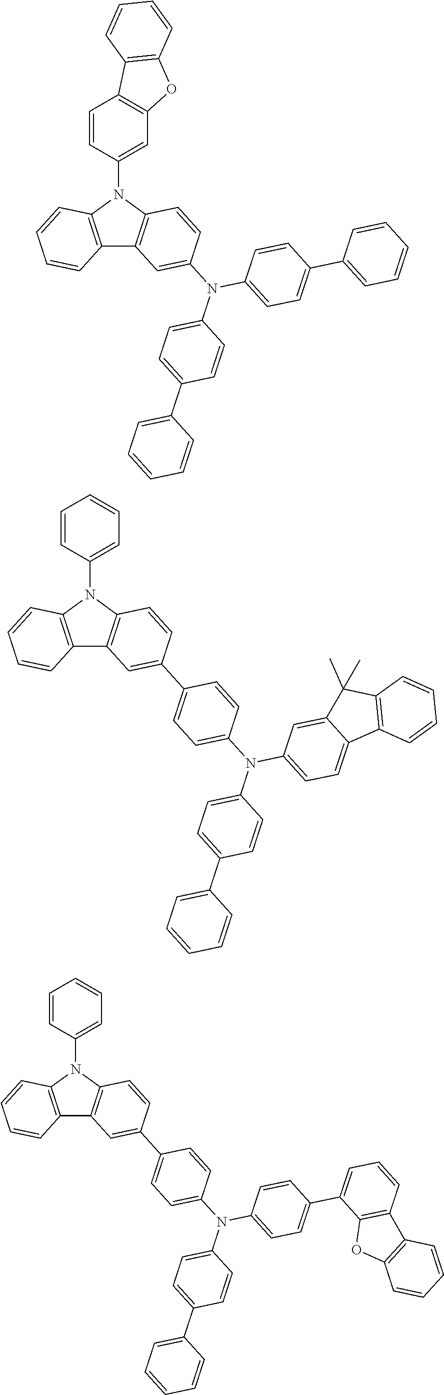

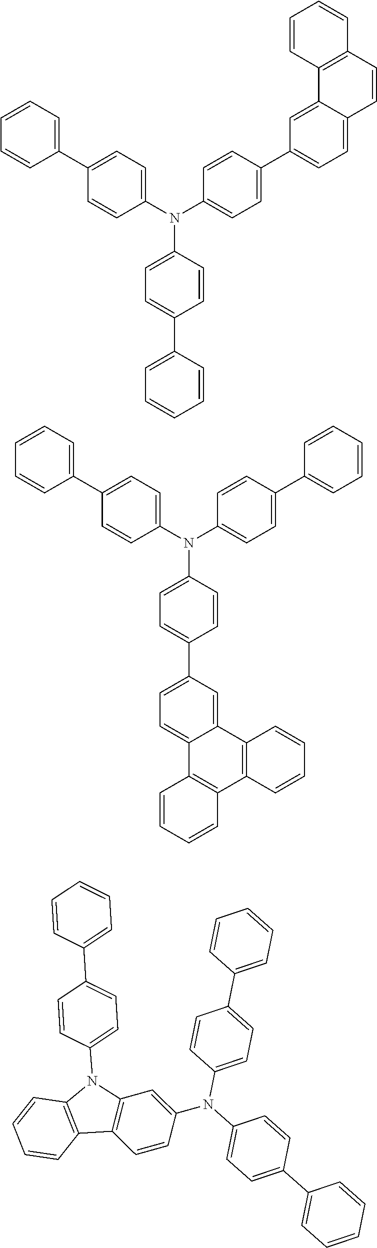 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

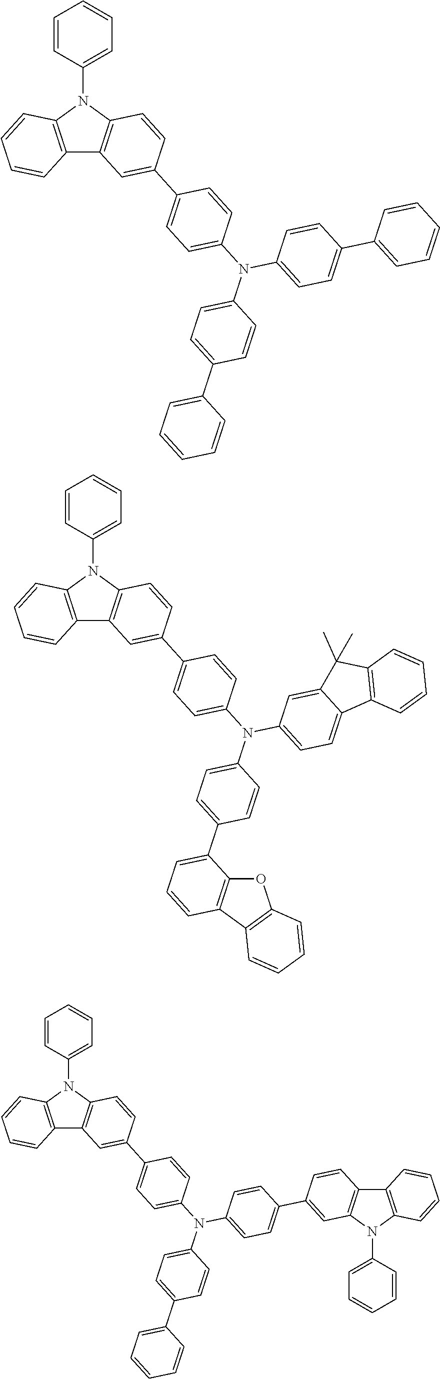 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

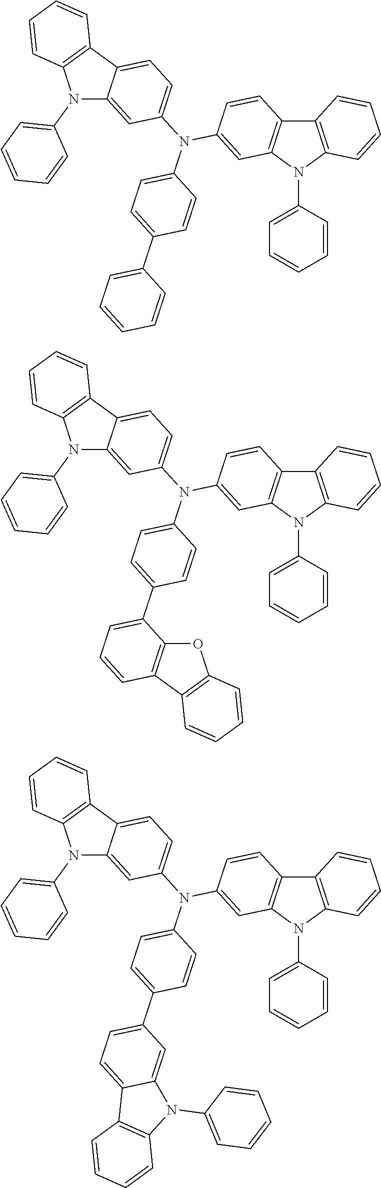 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

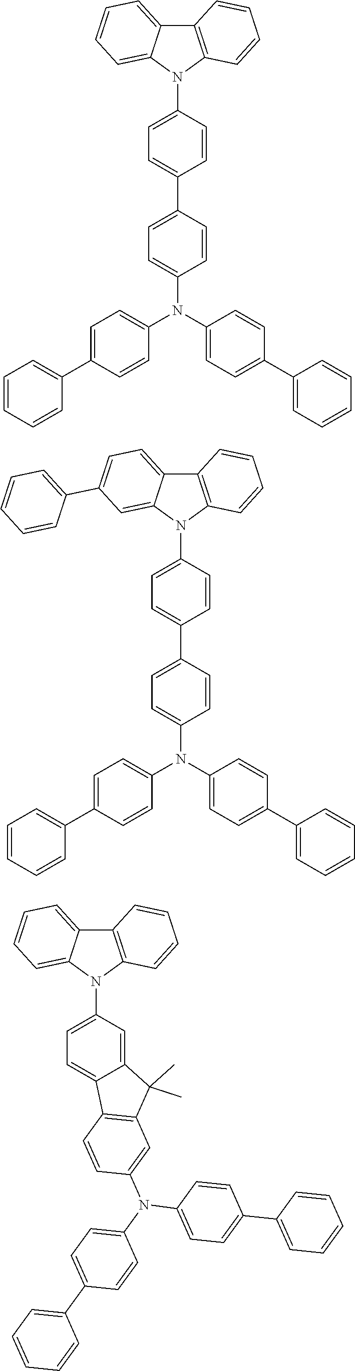

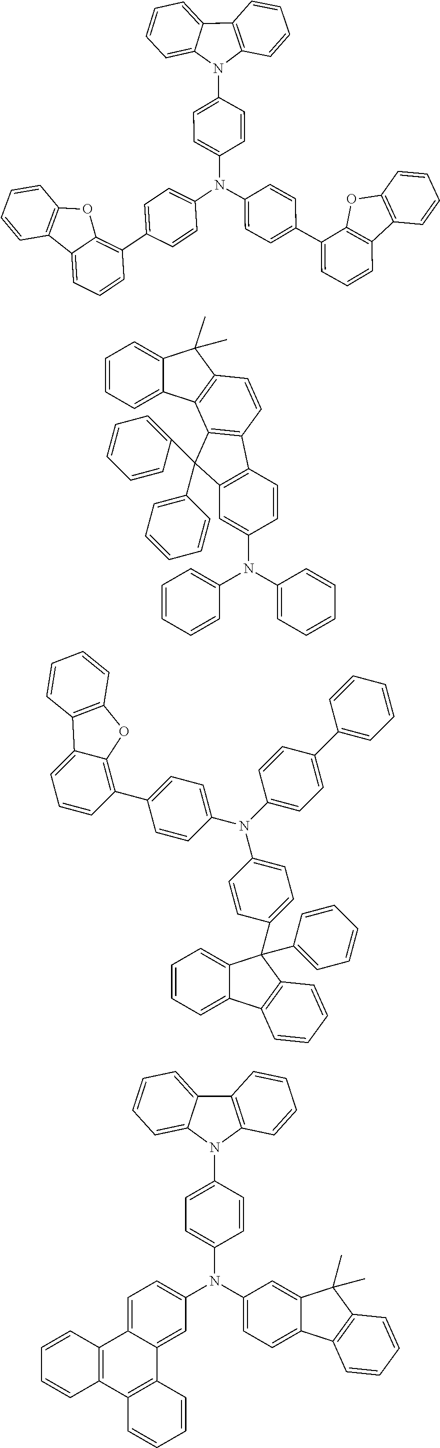

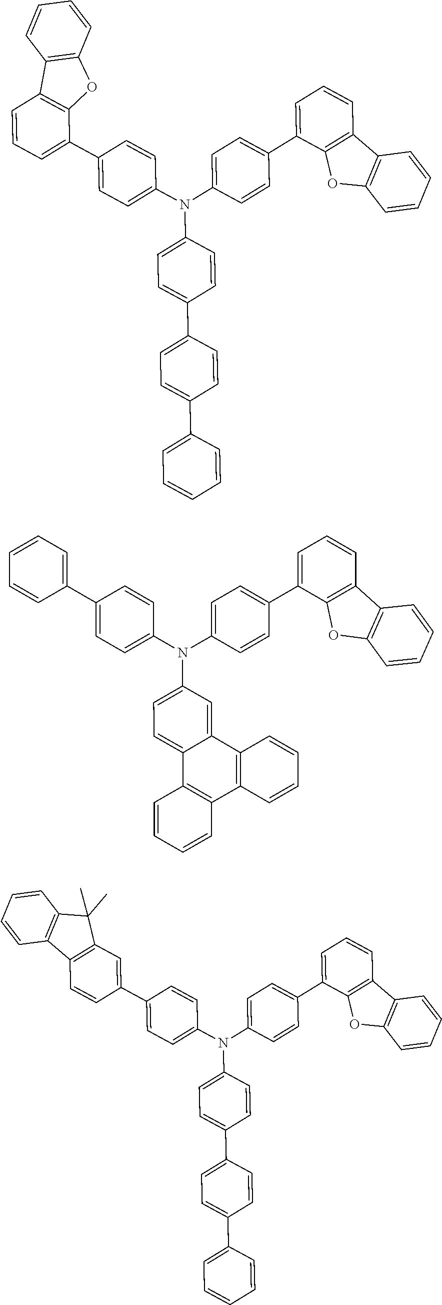 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(-c3ccc(N(c4ccc(-c5ccc(-c6ccccc6)cc5)cc4)c4ccc(-c5cccc6c5oc5ccccc56)cc4)cc3)cc21]

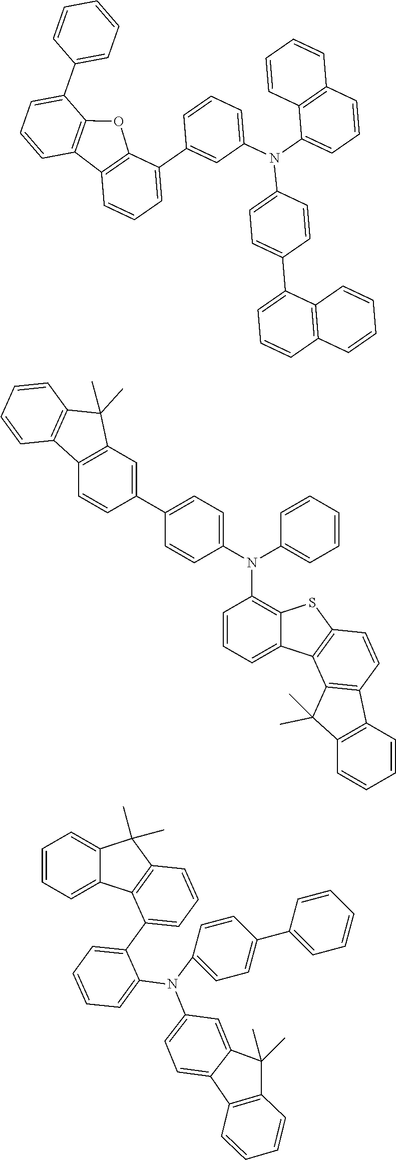 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

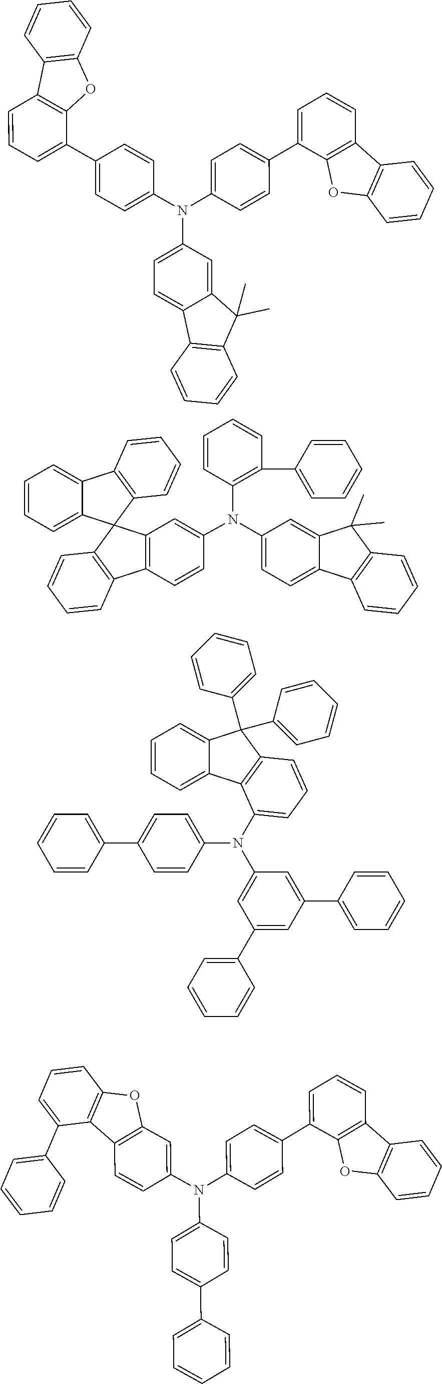

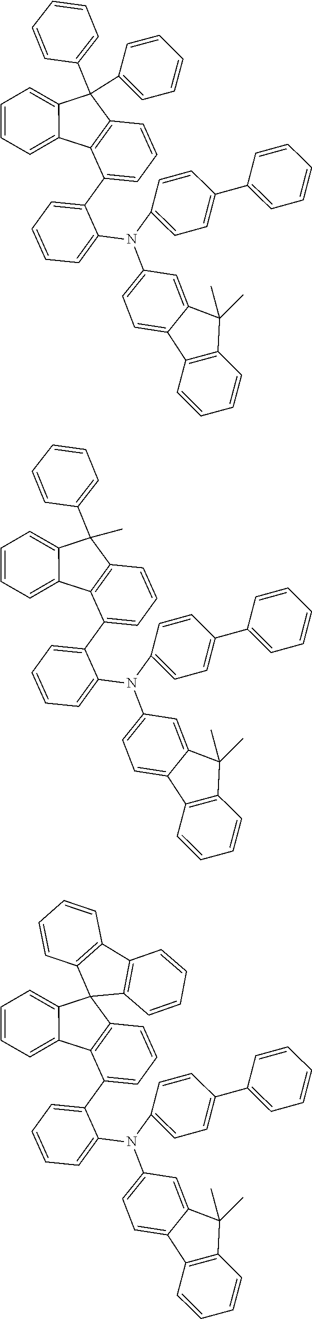 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

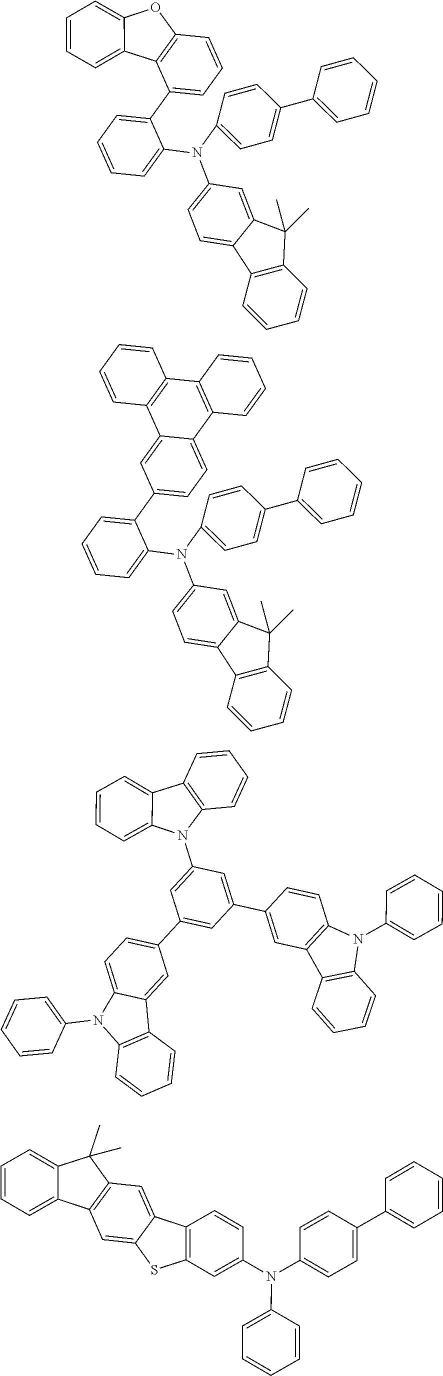

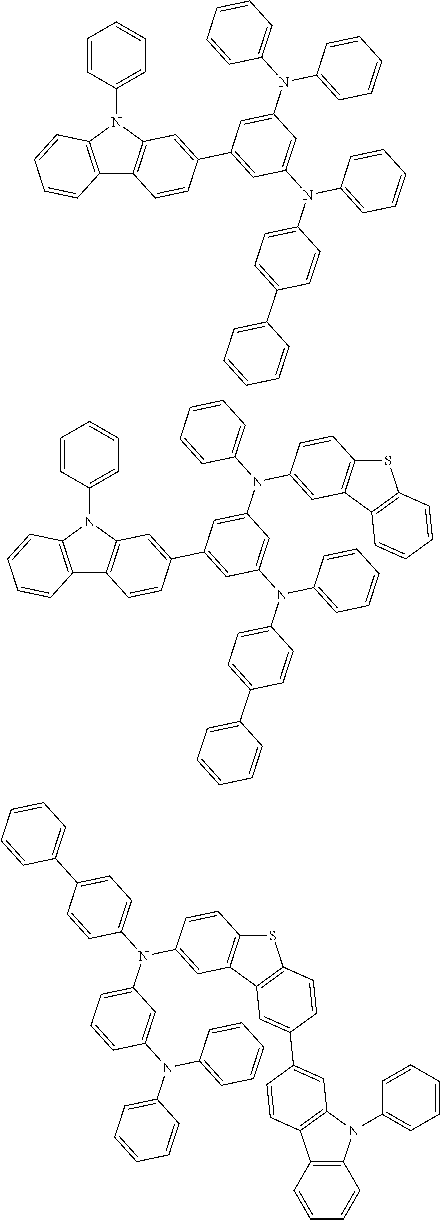

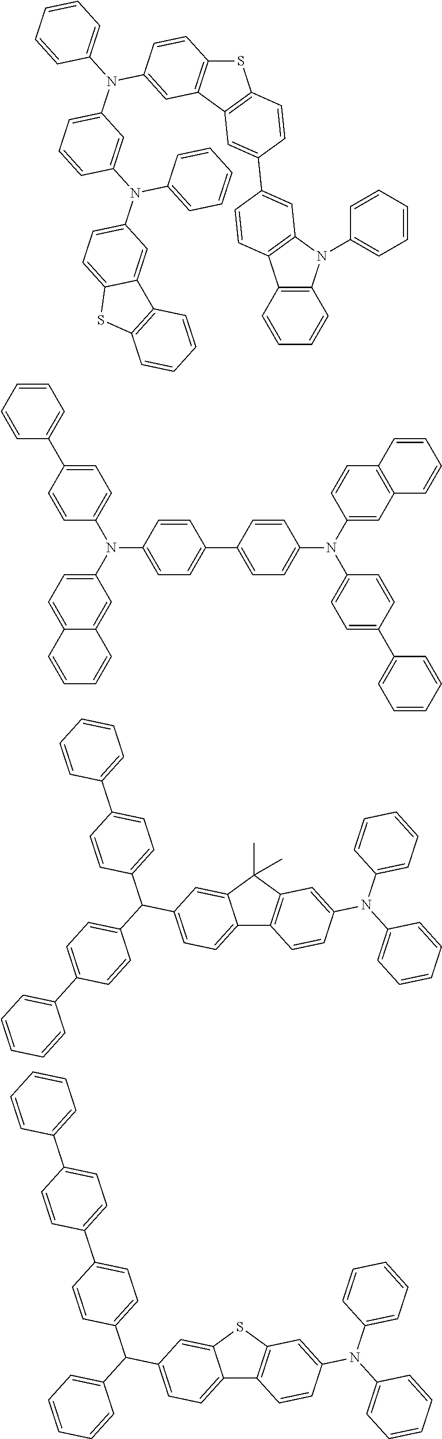

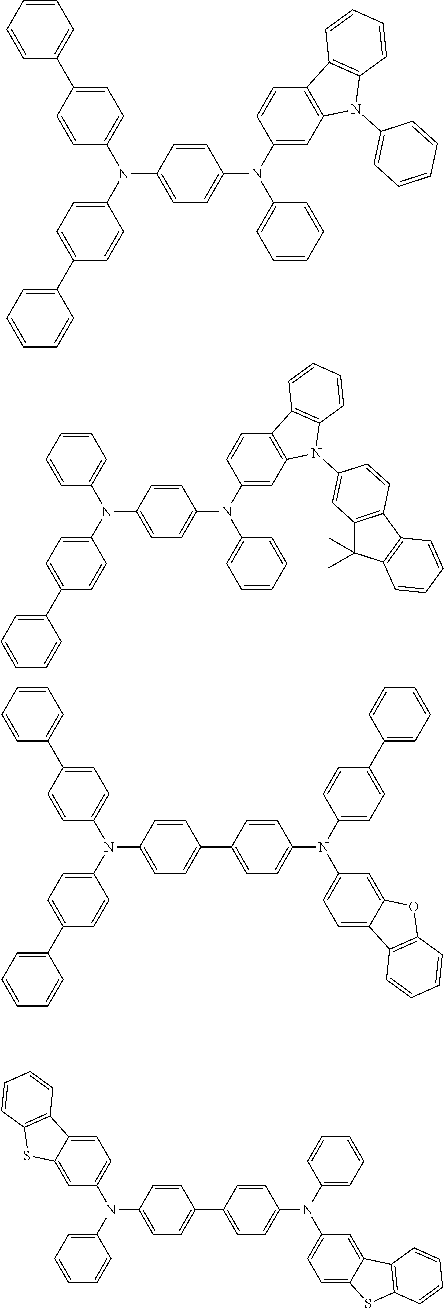

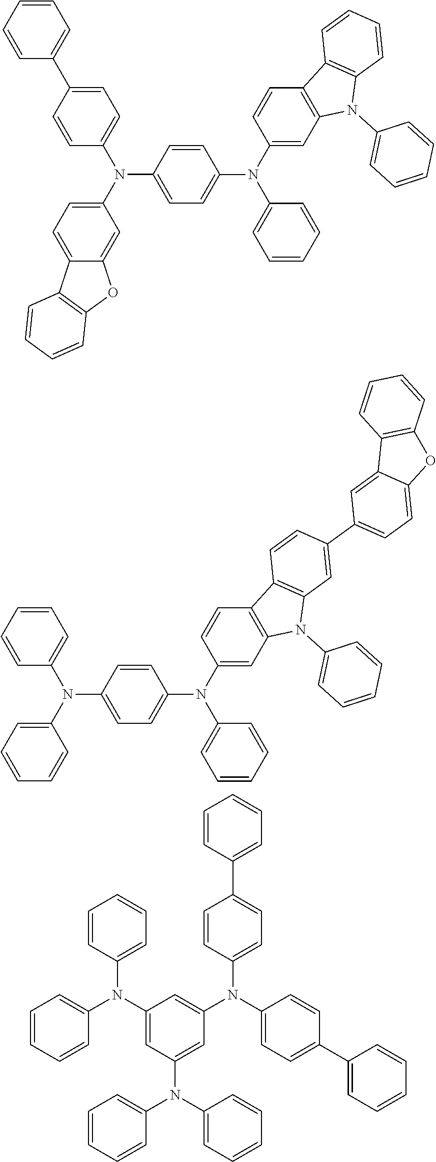

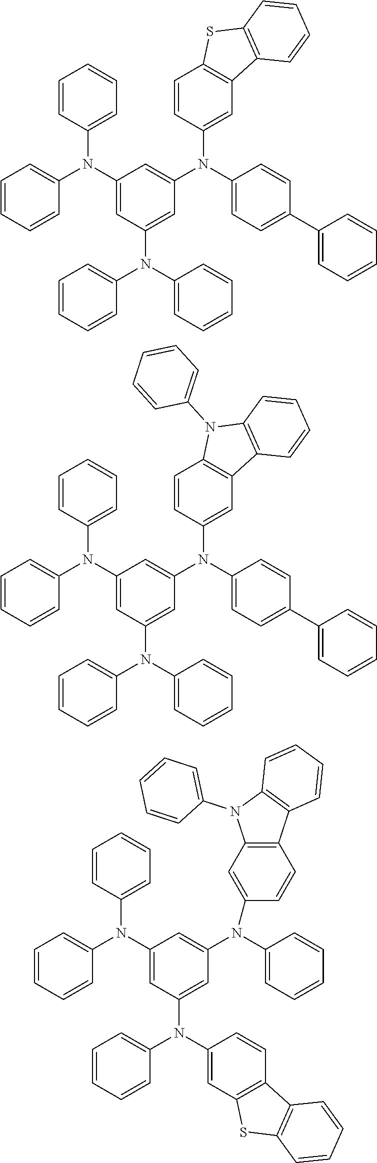

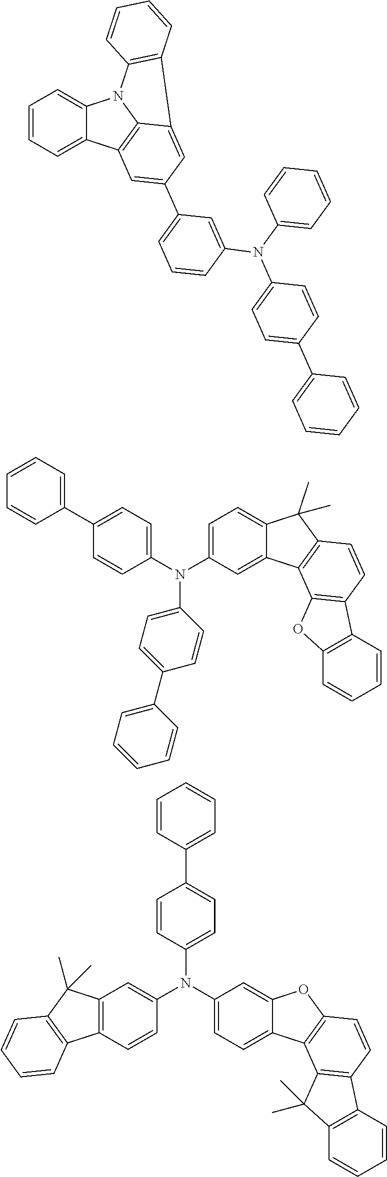

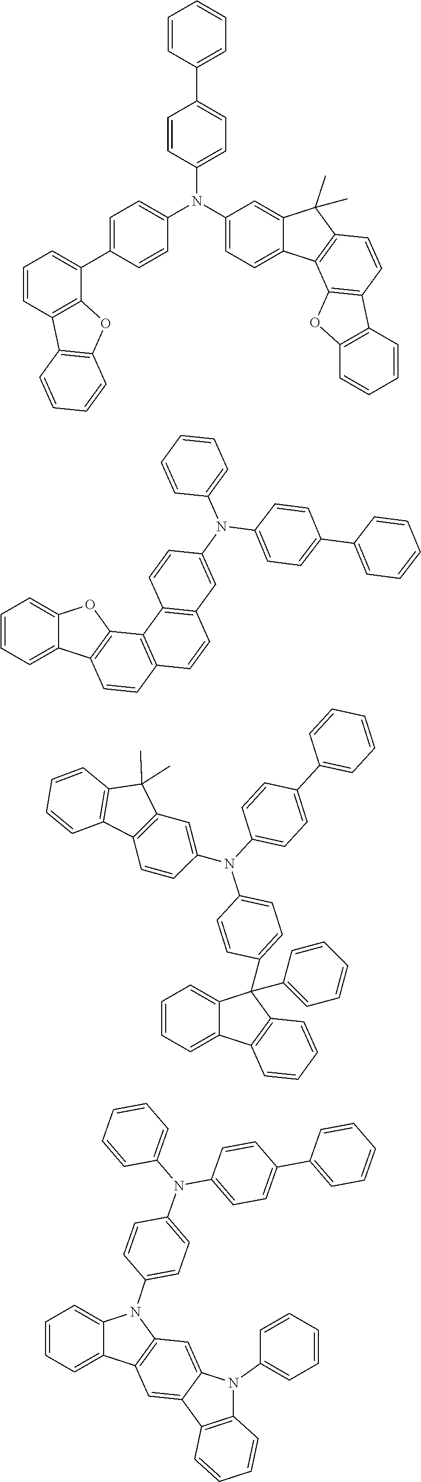 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

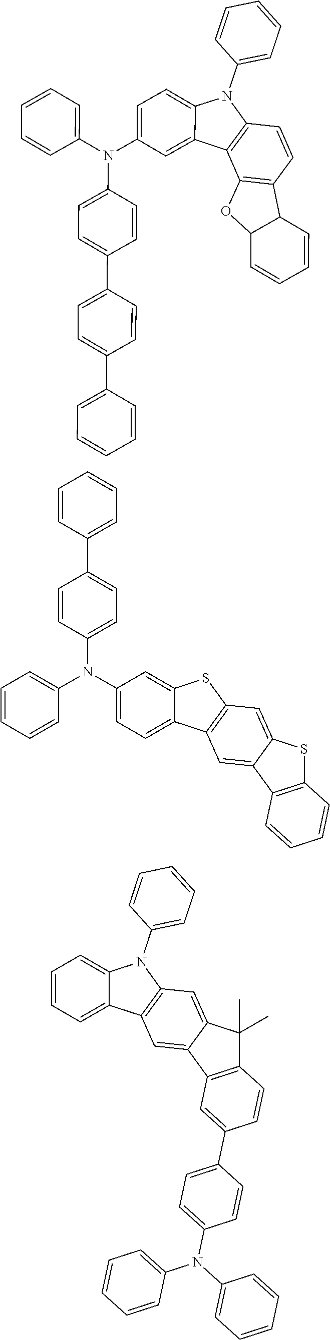

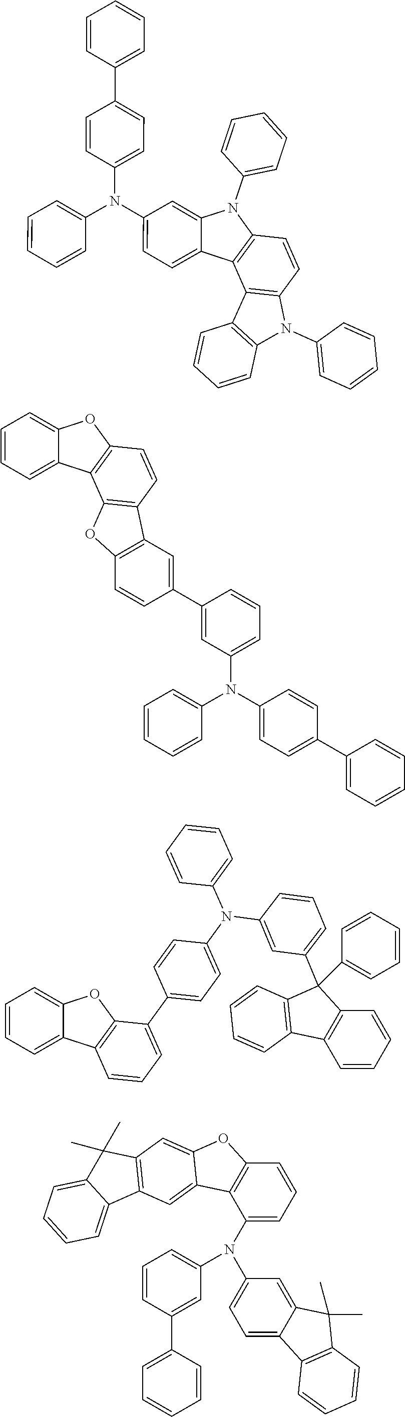

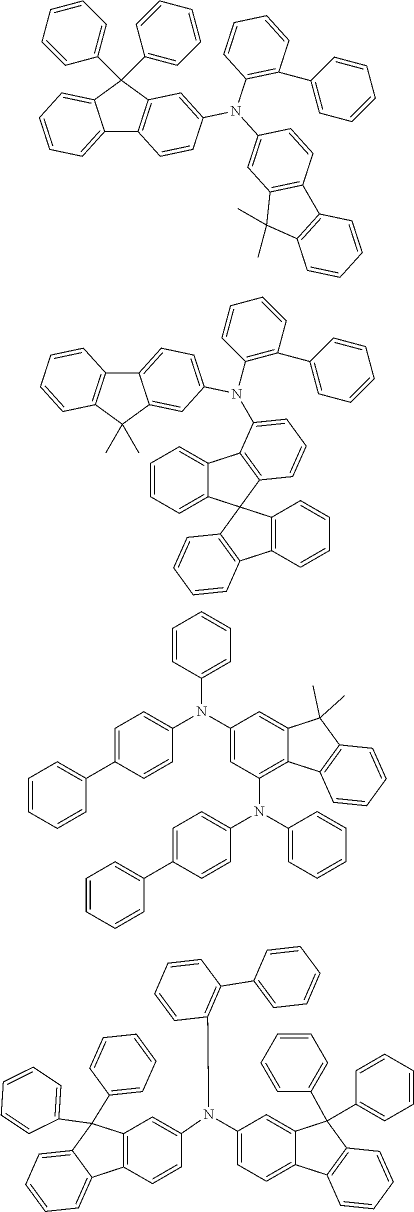

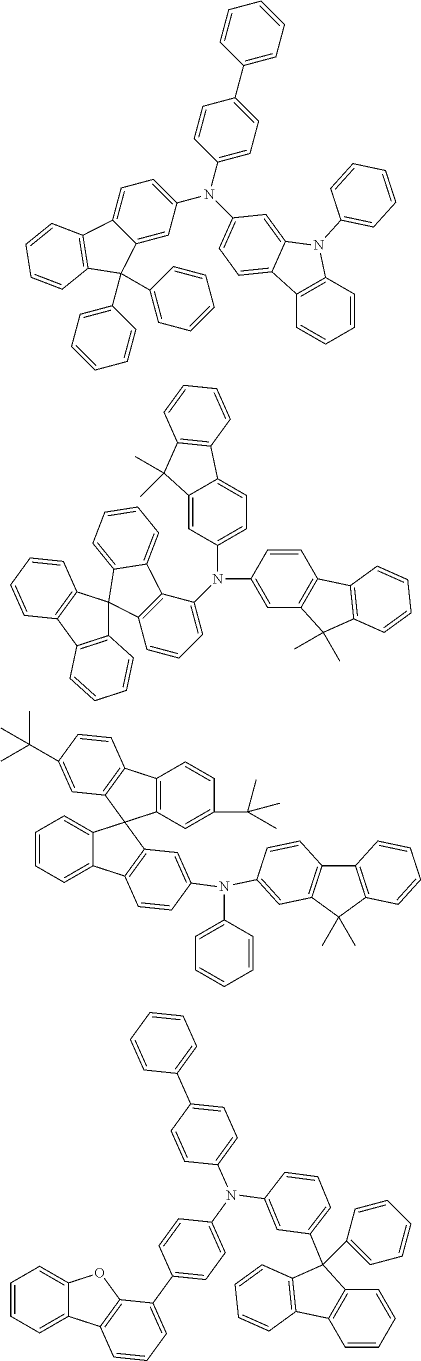

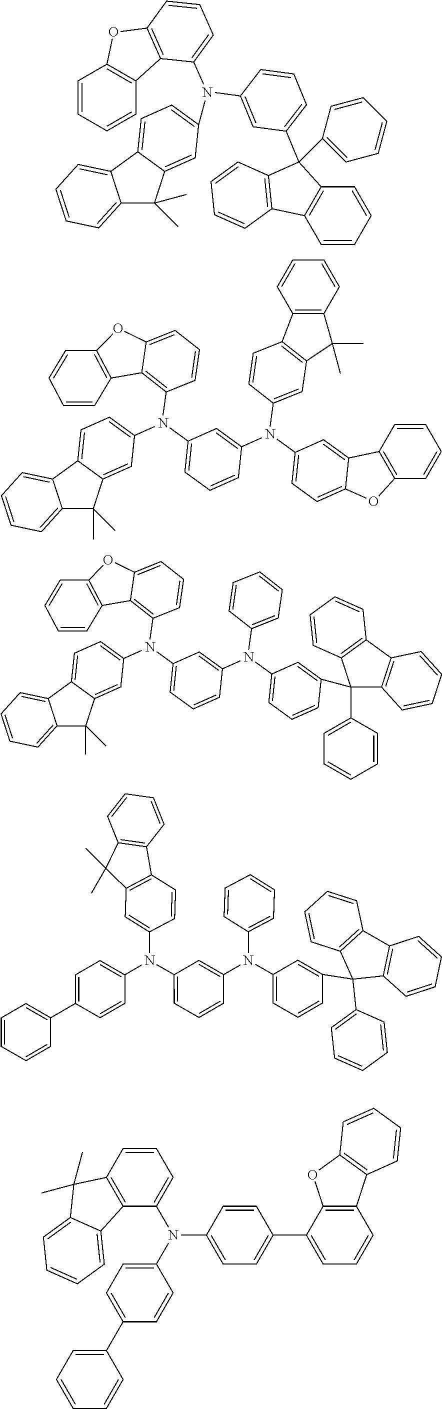

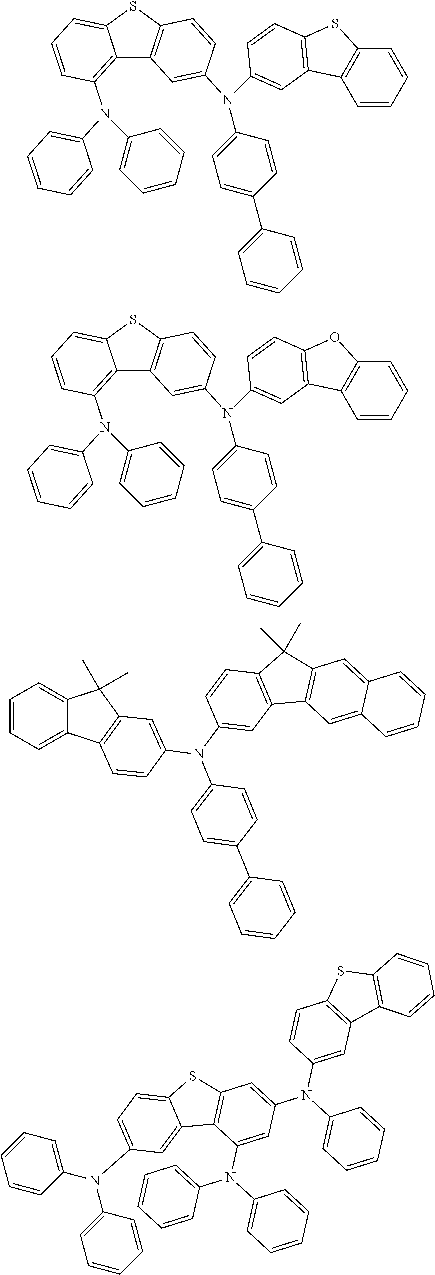

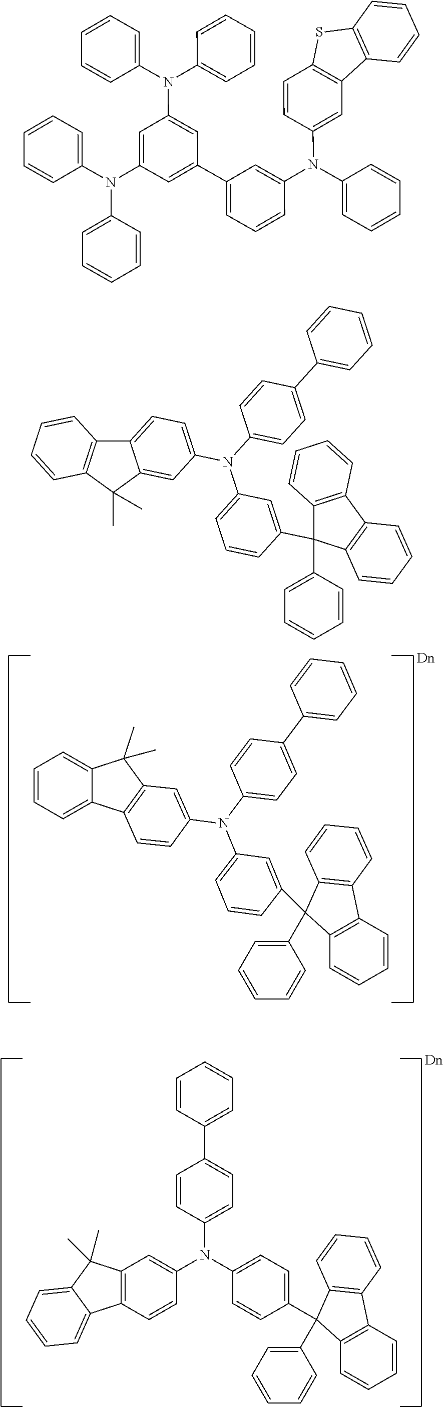

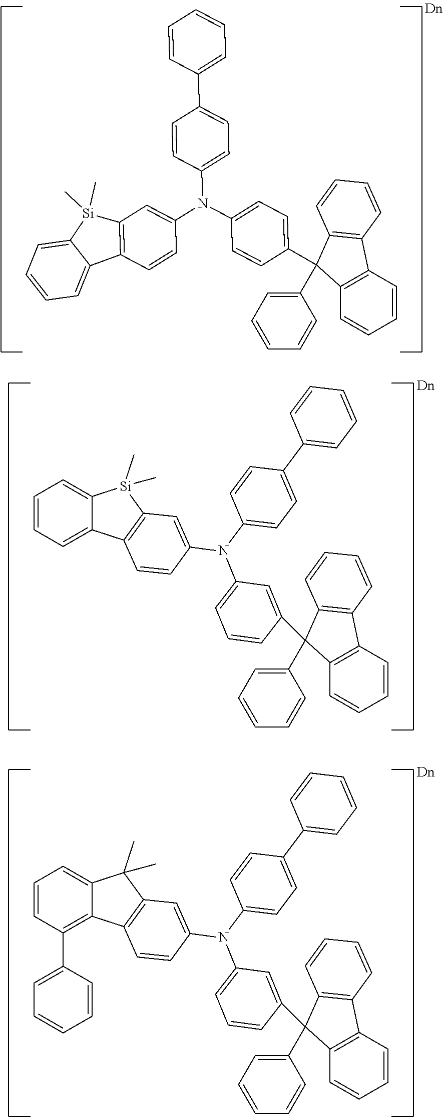

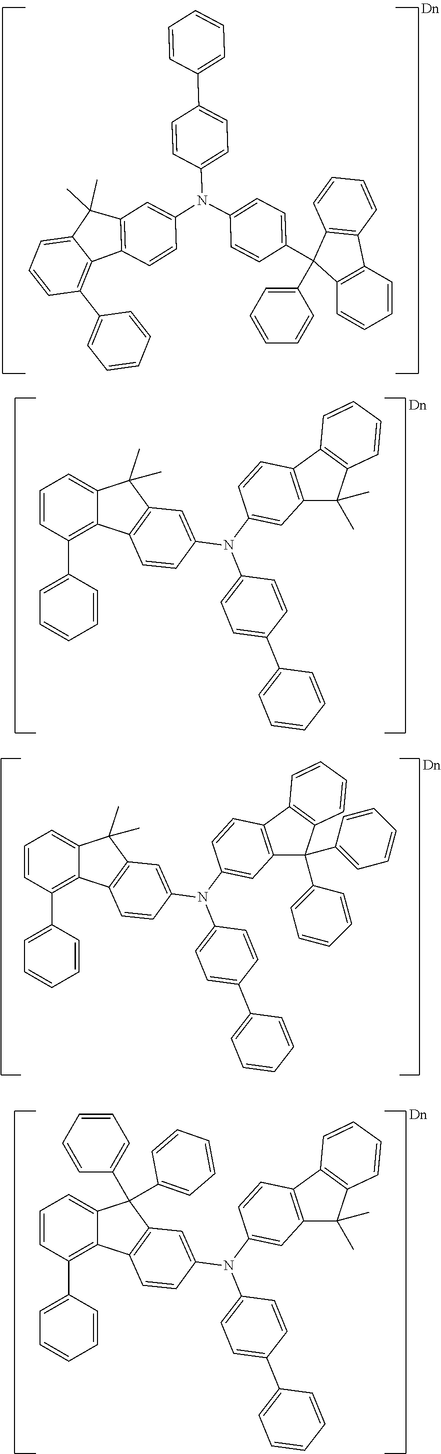

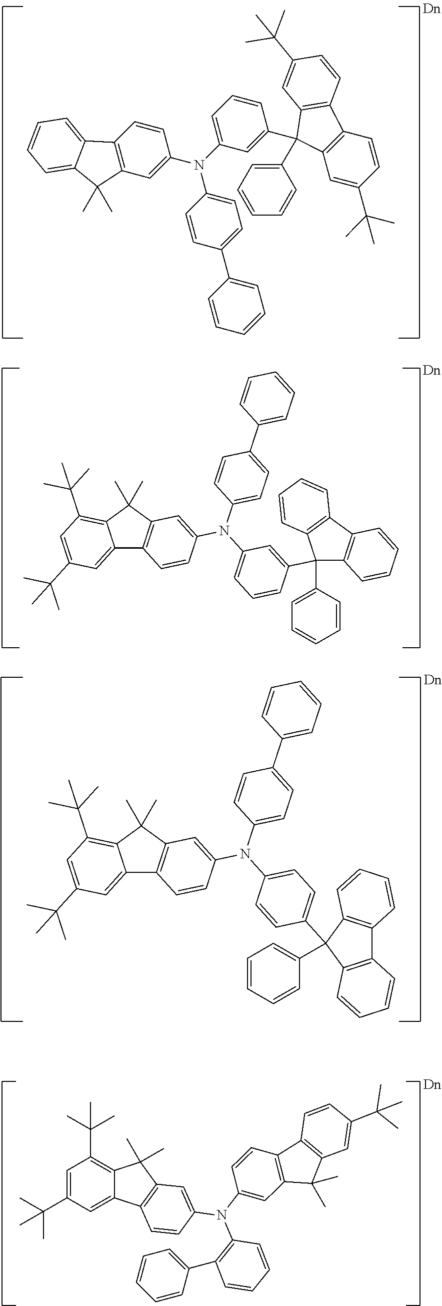

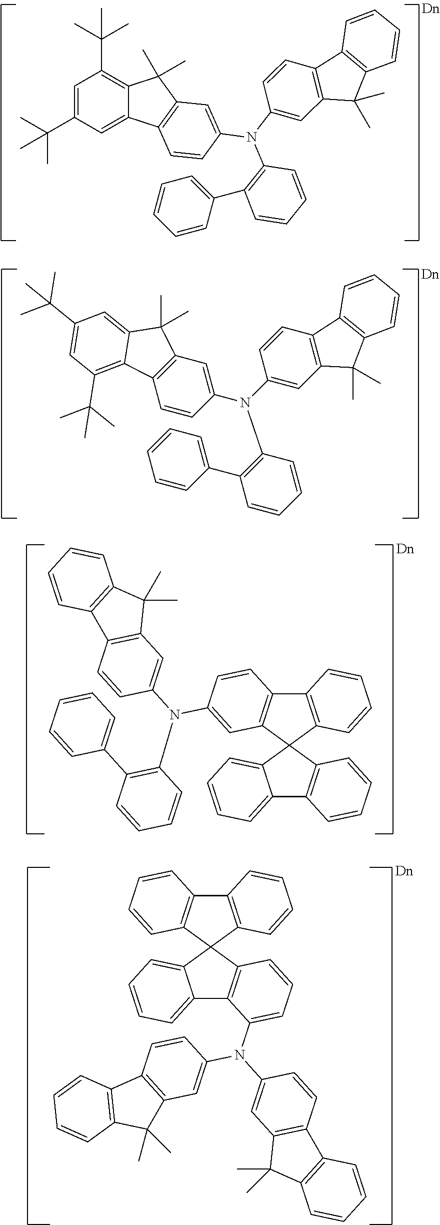

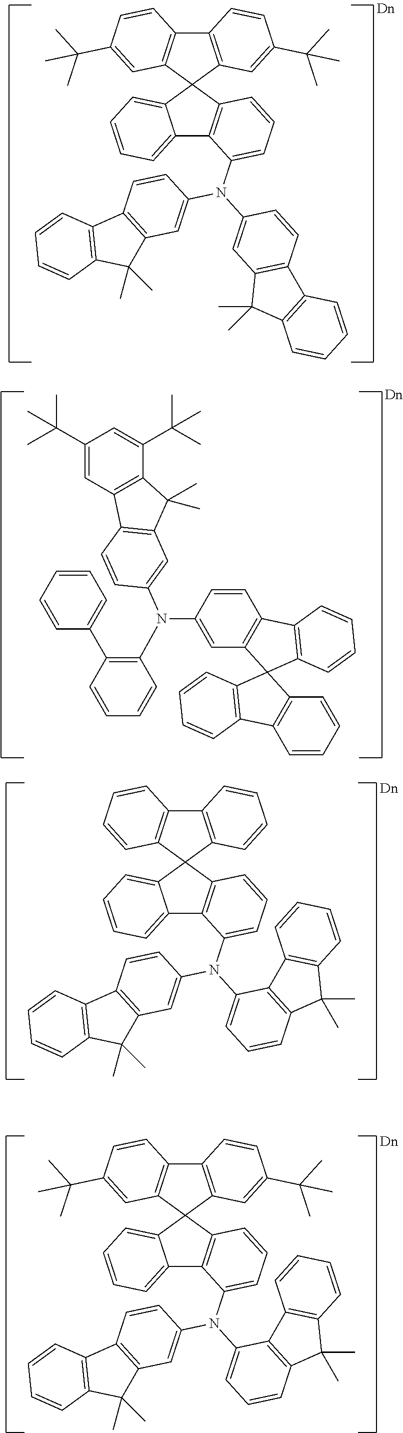

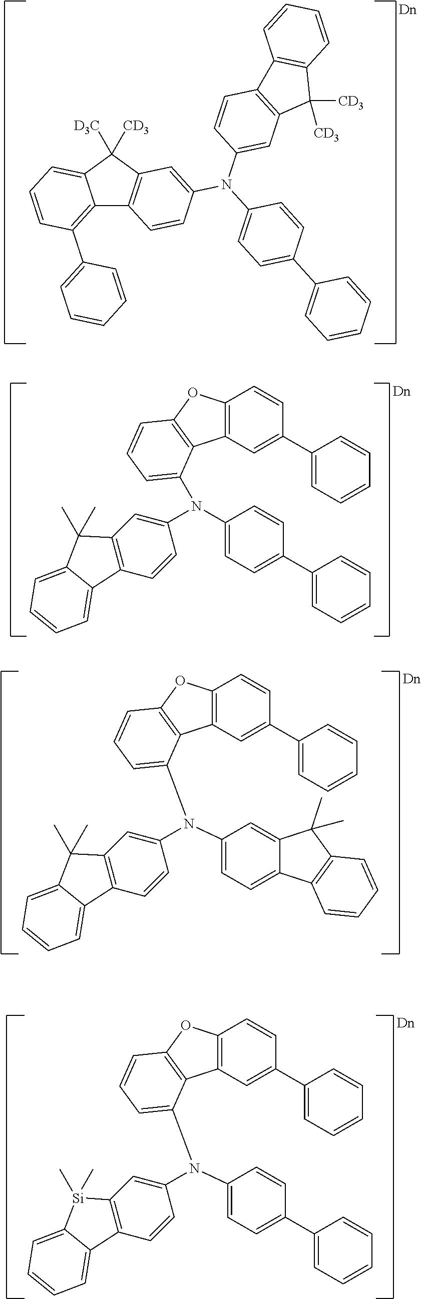

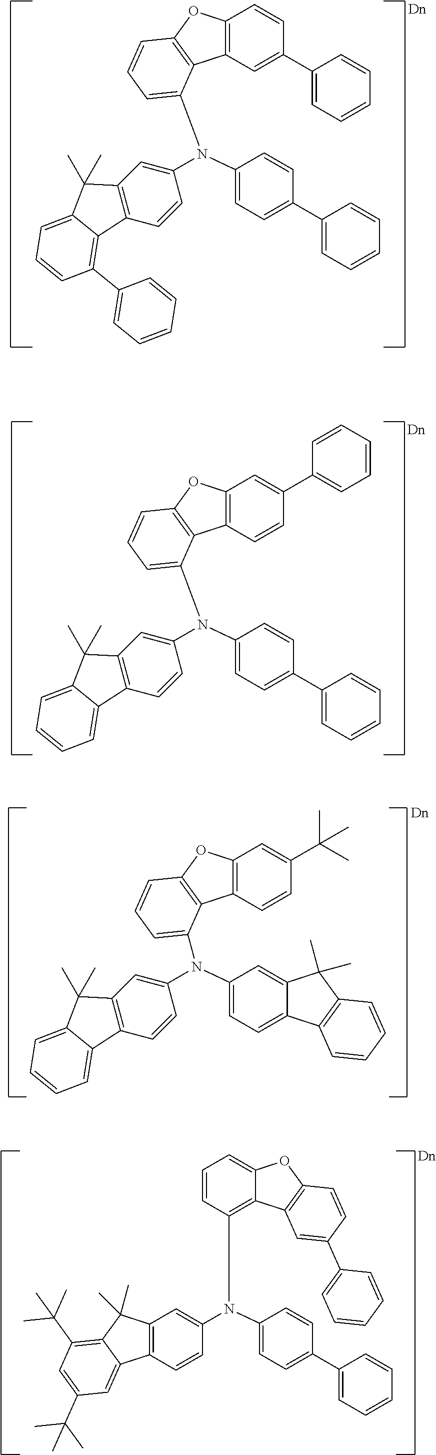

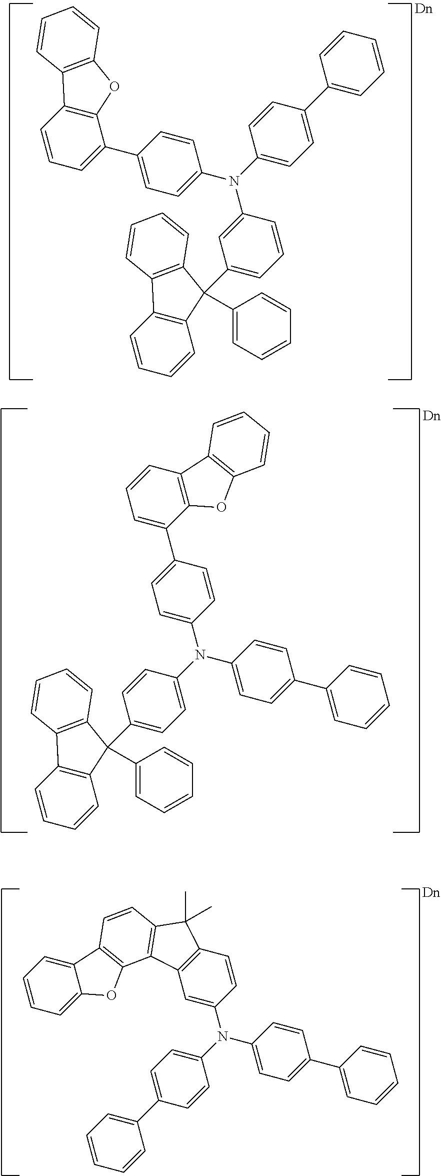

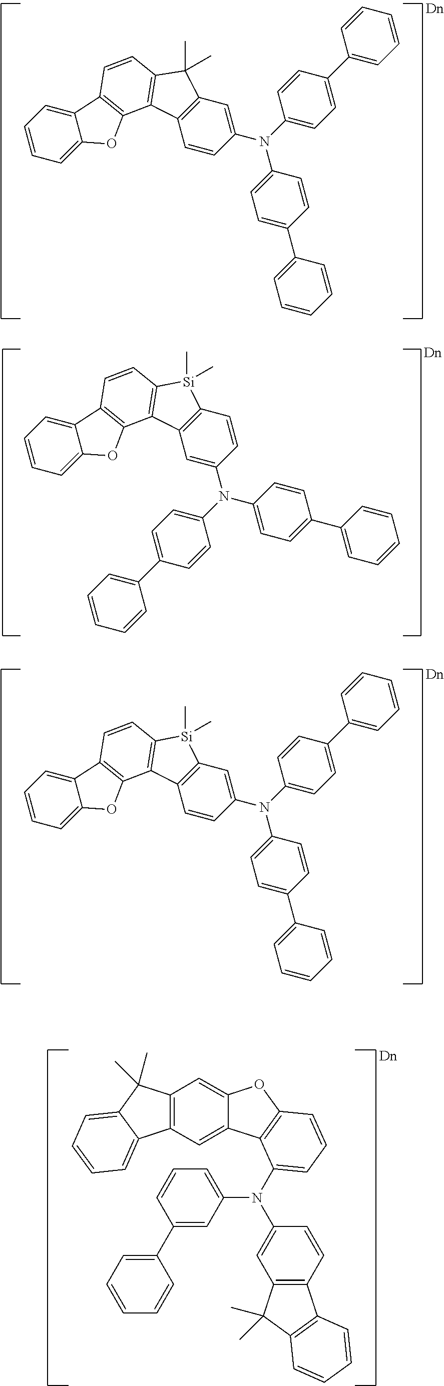

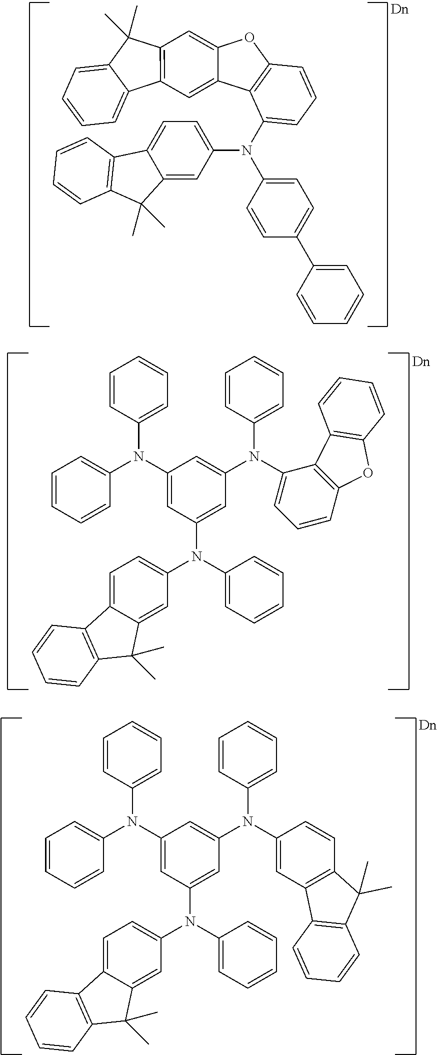

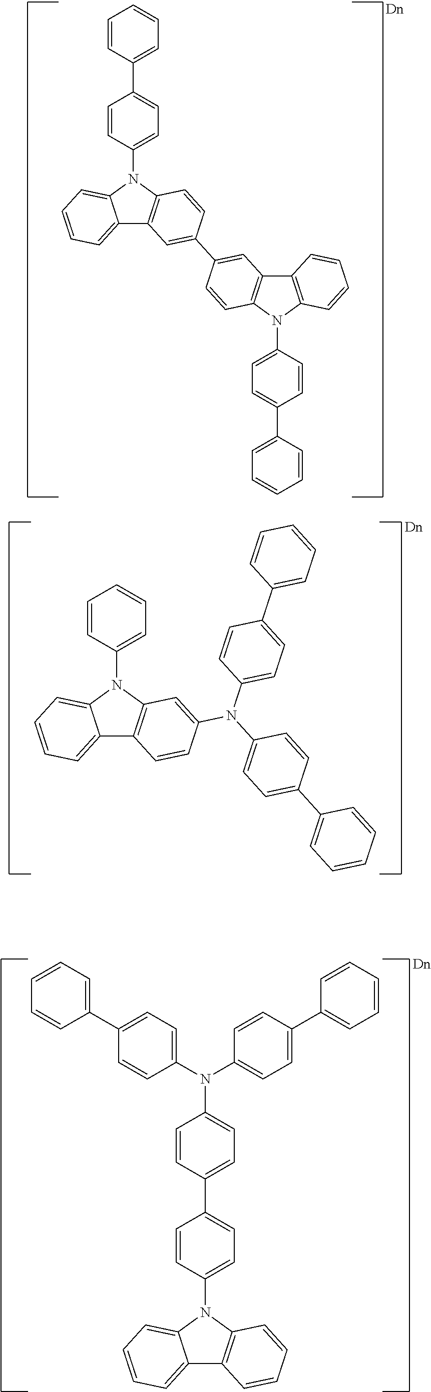

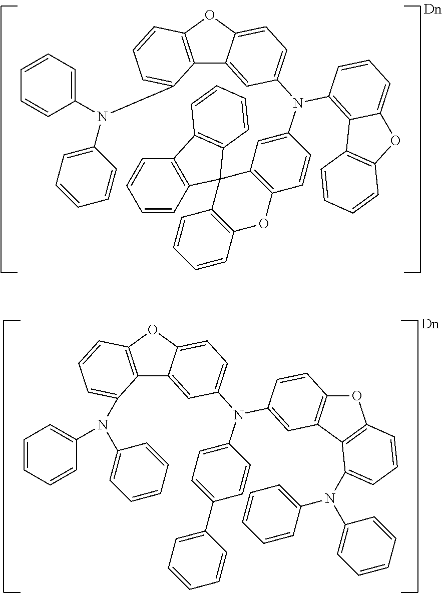

(Dn refers to the number of deuterium substitutions and indicates a structure substituted with one or more deuterium atoms). However, as noted above, any hydrogen in any compound may be protium, deuterium, or tritium, based on natural or artificial substitution.

In the hole transport region, in addition to the compounds described above, other suitable compounds and compounds having a similar structure may also be used.

In an implementation, the charge transport region may be, e.g., the electron transport region 150.

The electron transport region 150 may help further increase electron injection and/or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

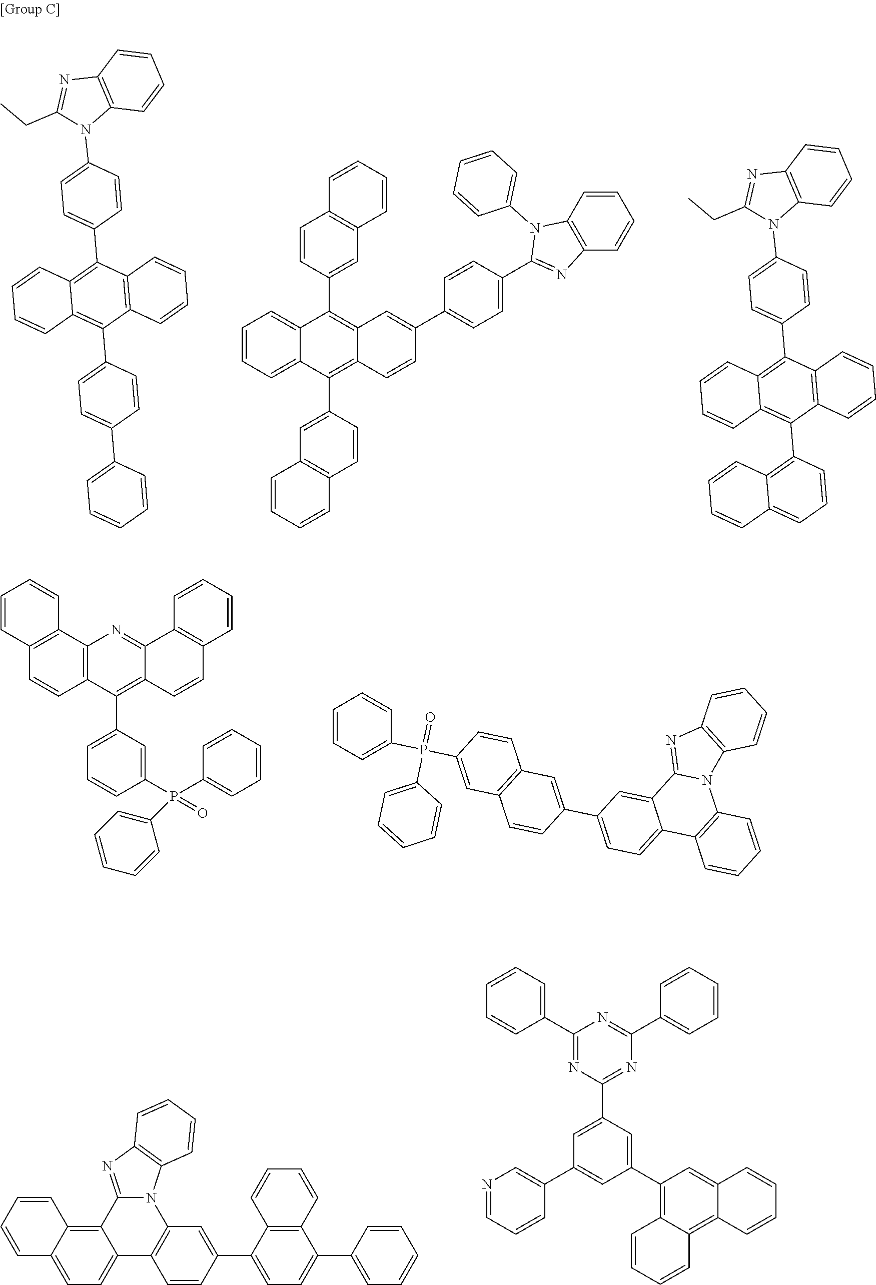

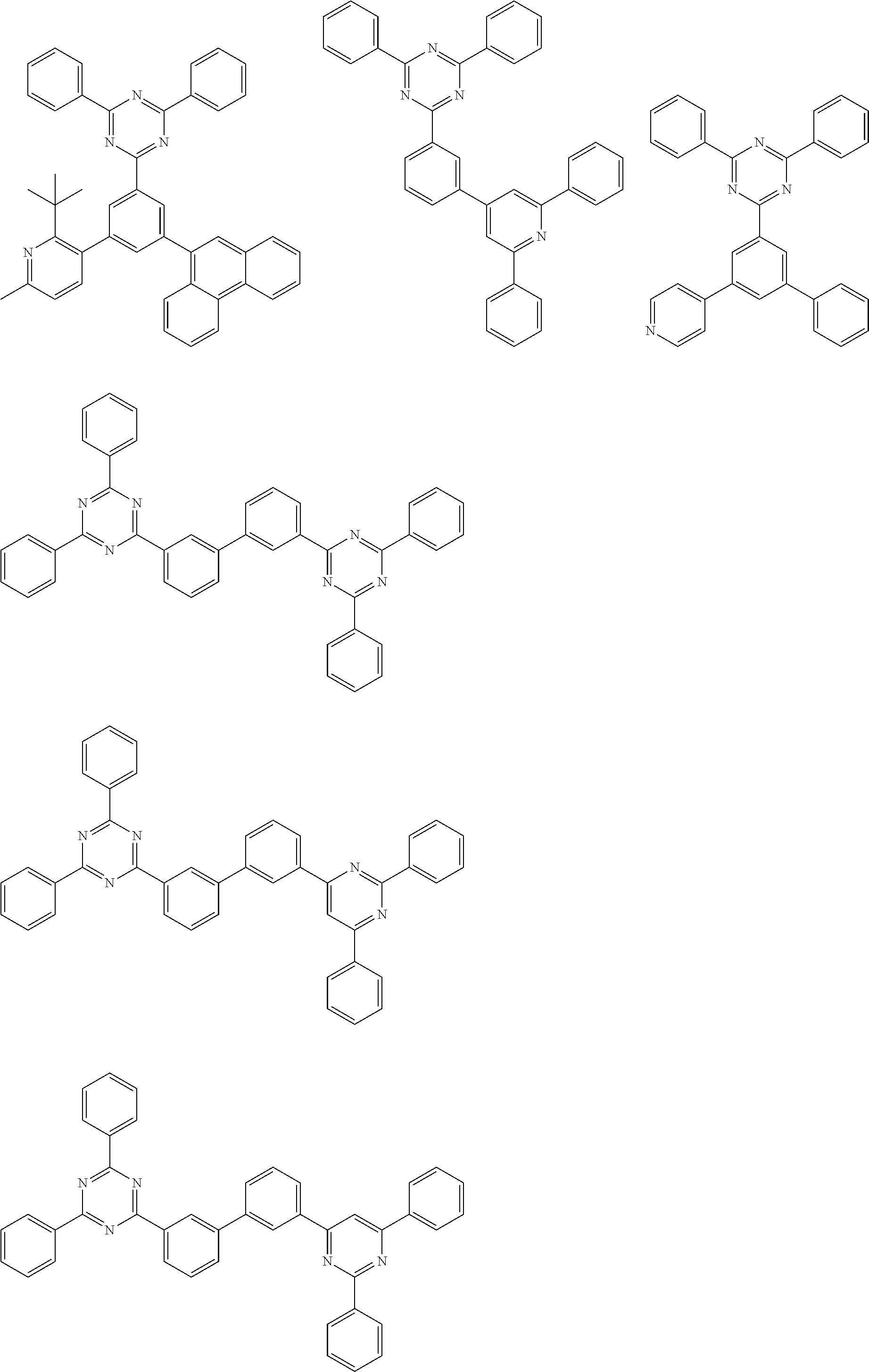

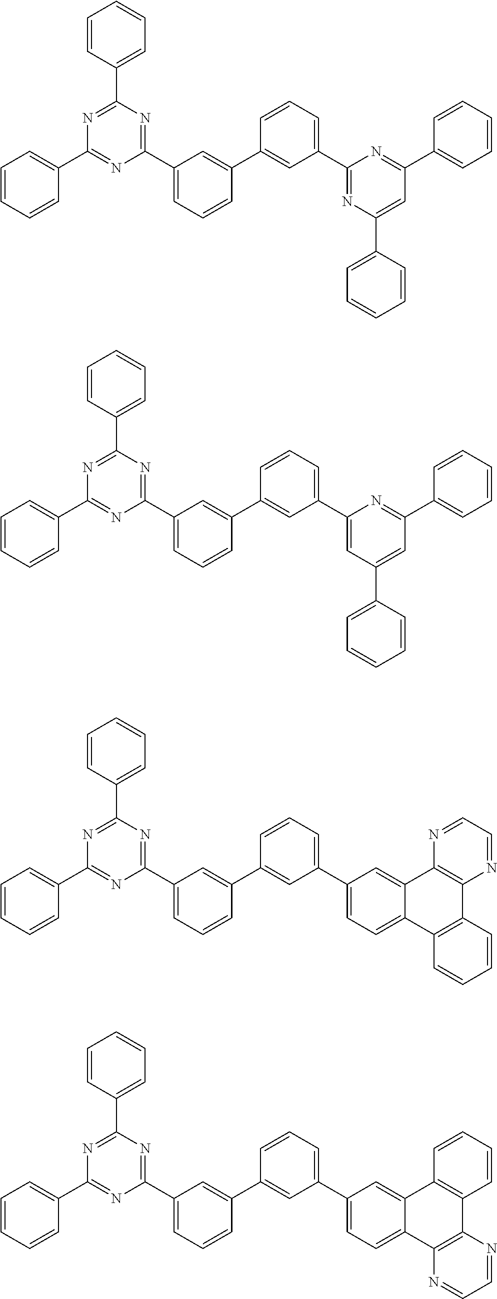 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

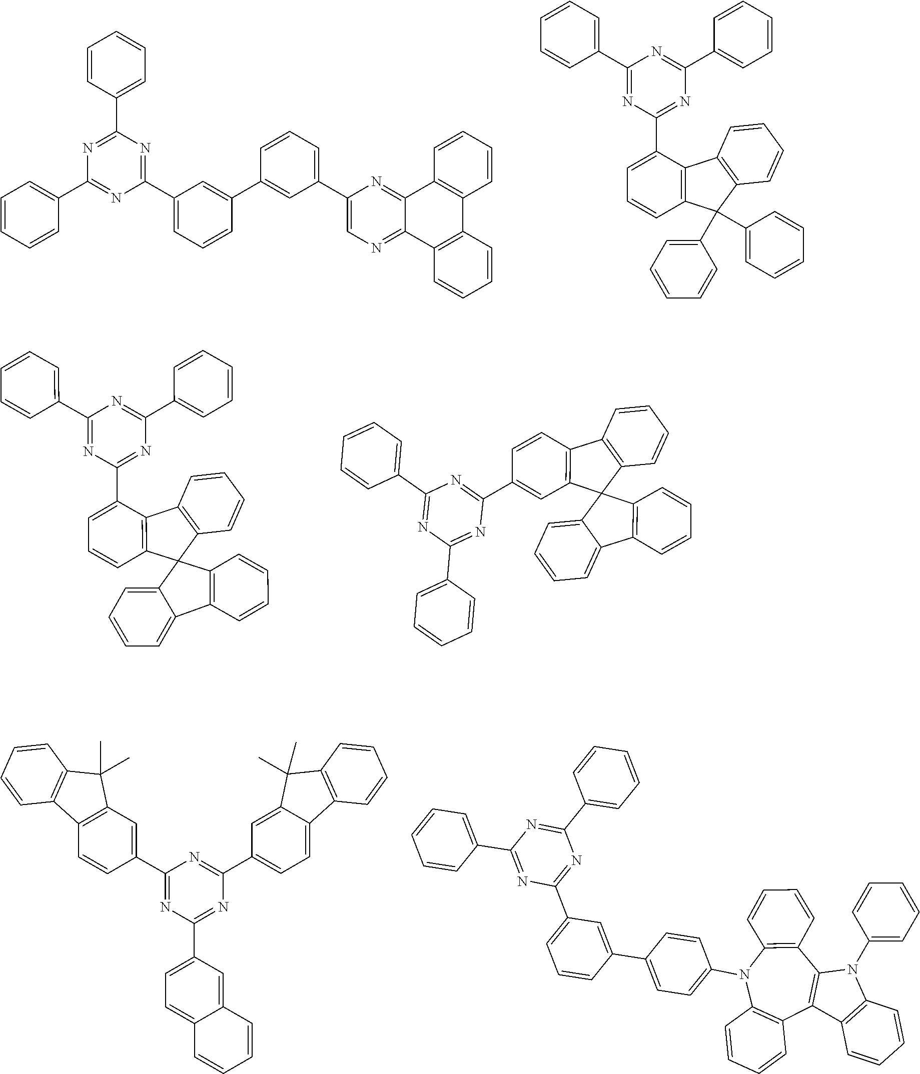 [SMILES: c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1 ; CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

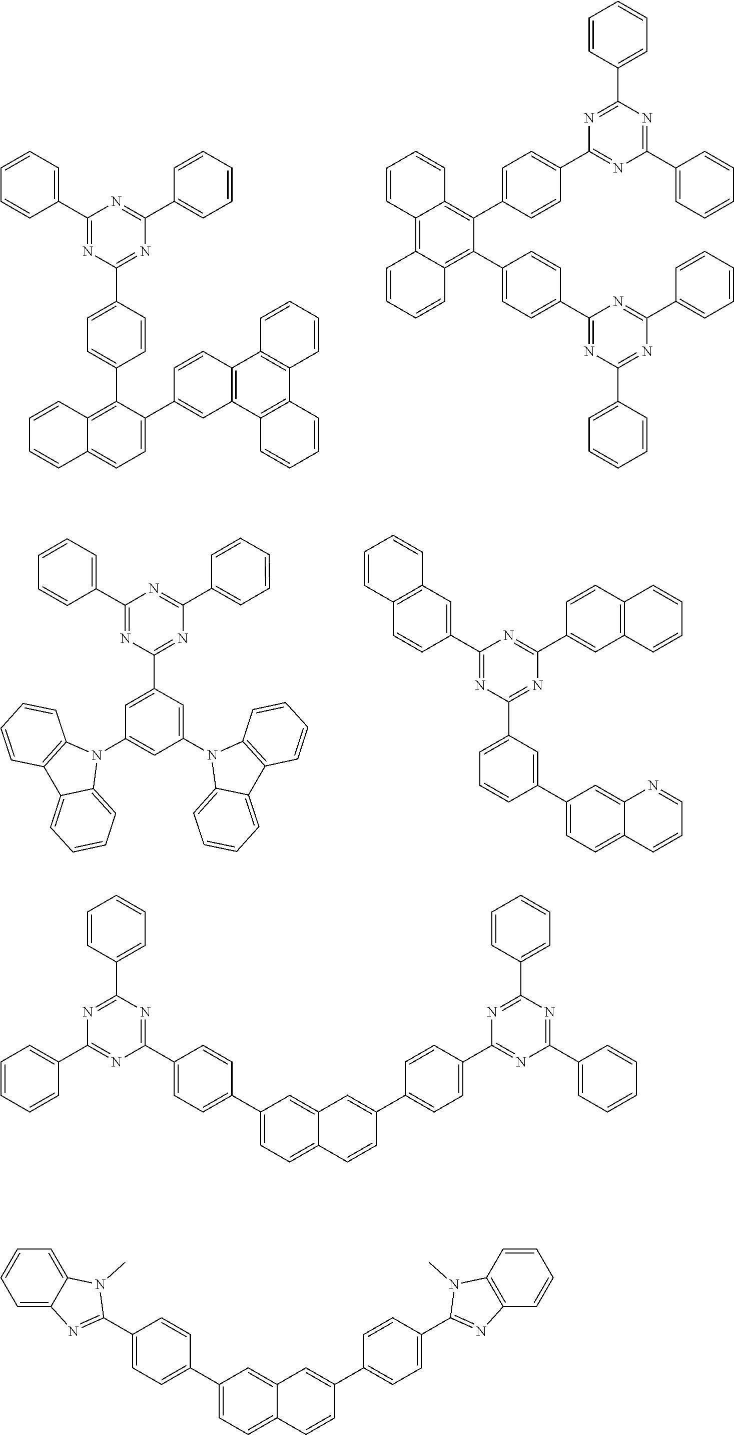

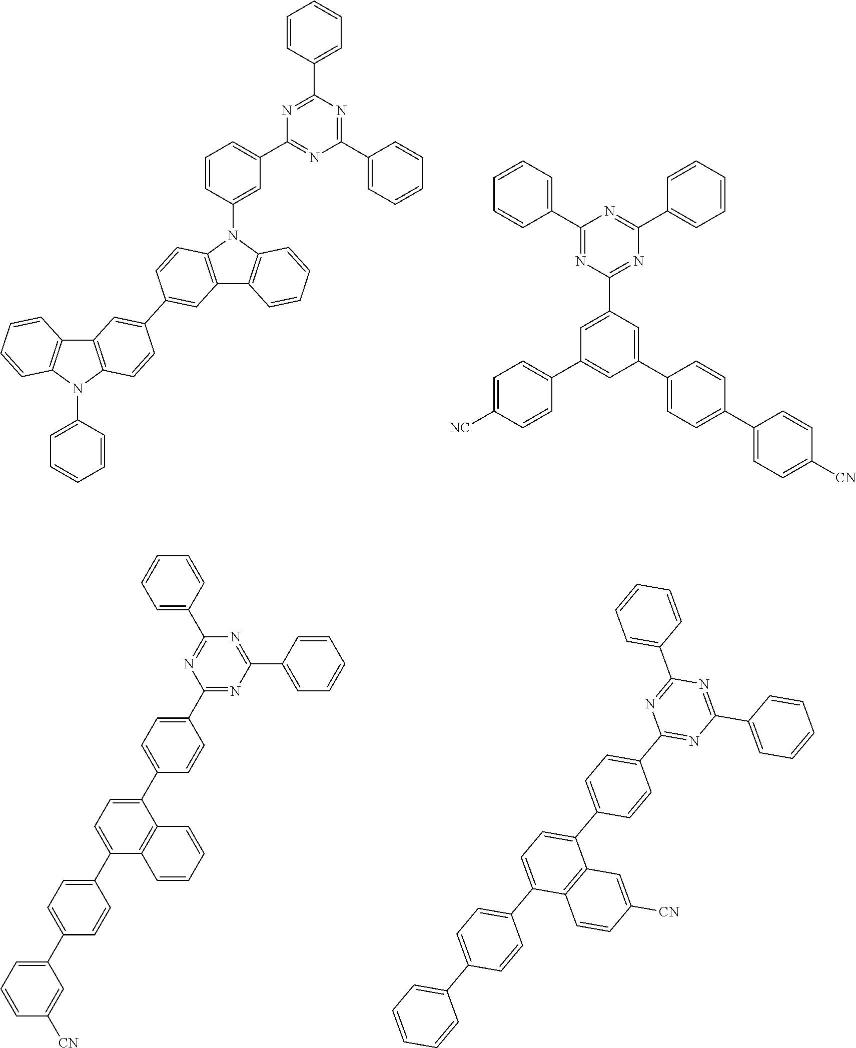 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

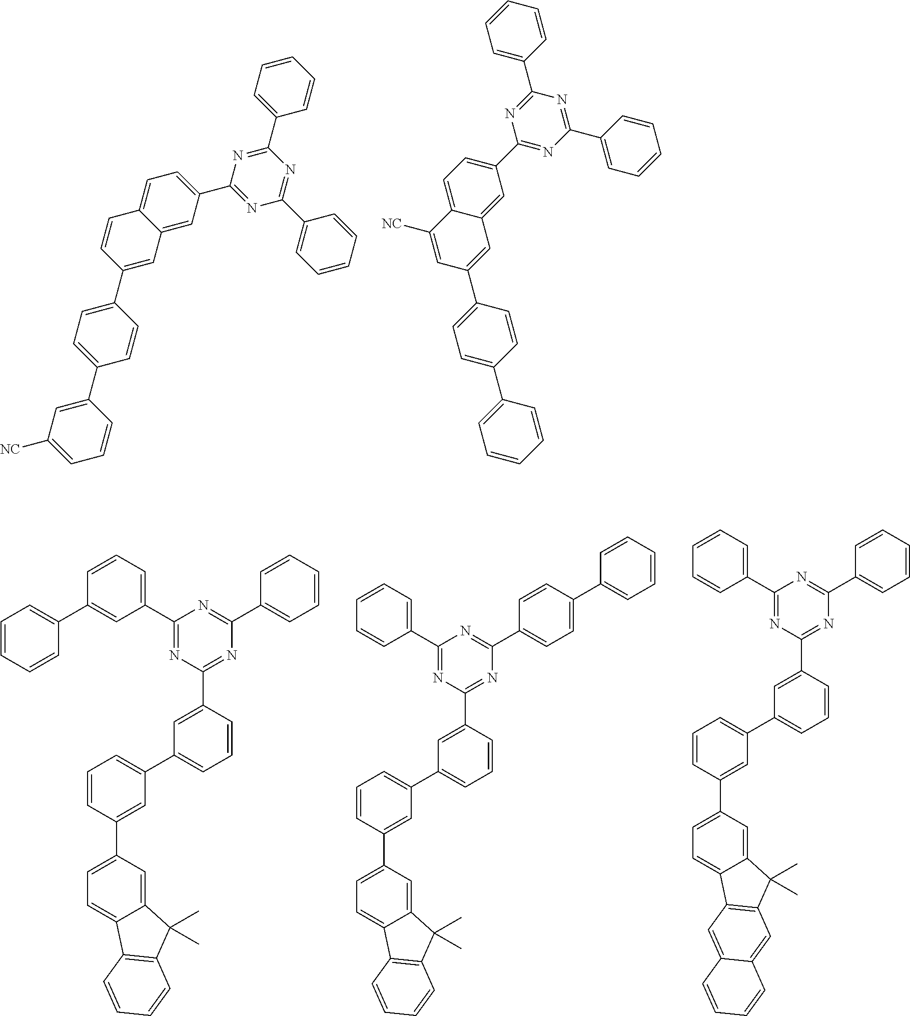

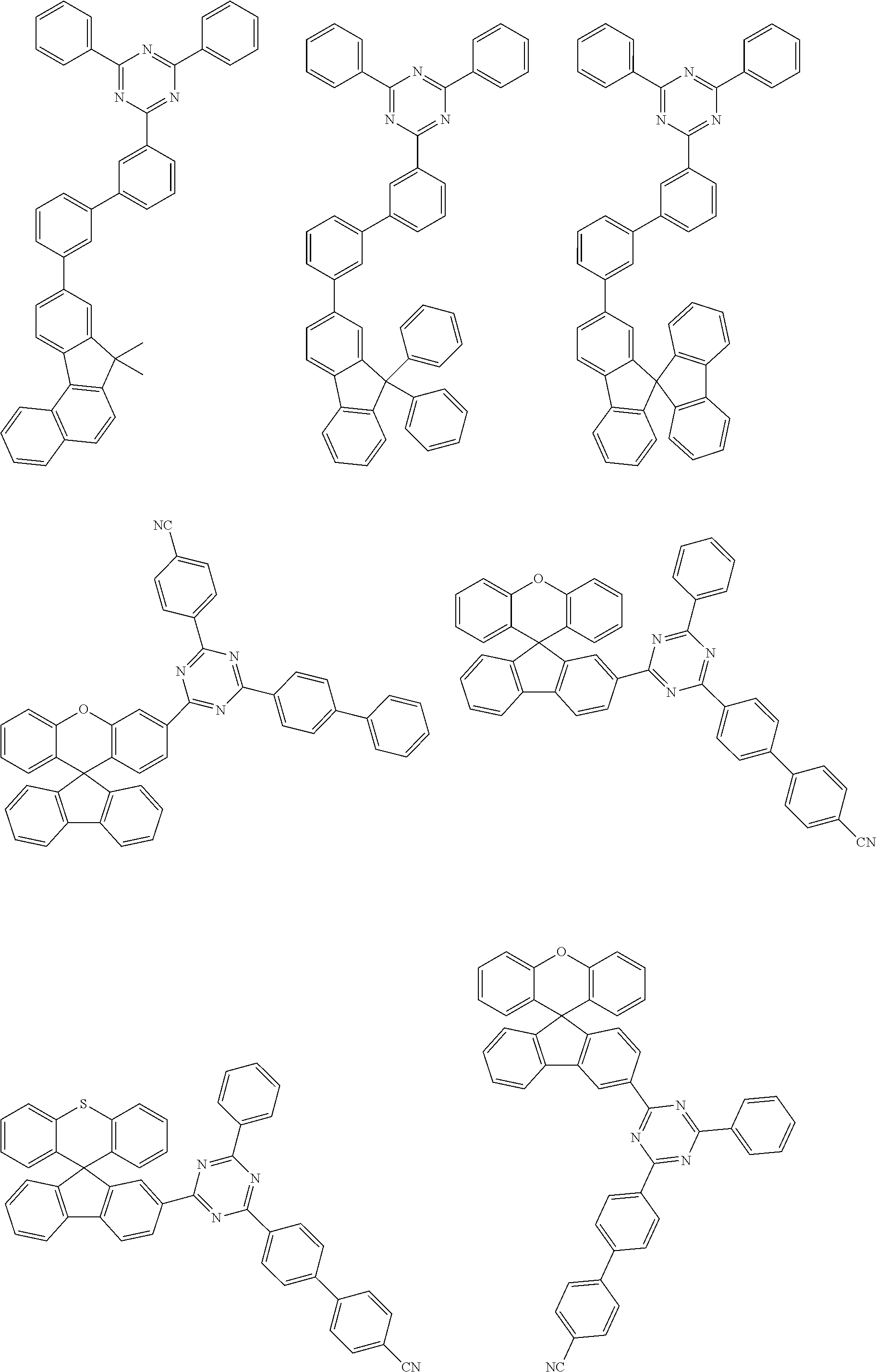

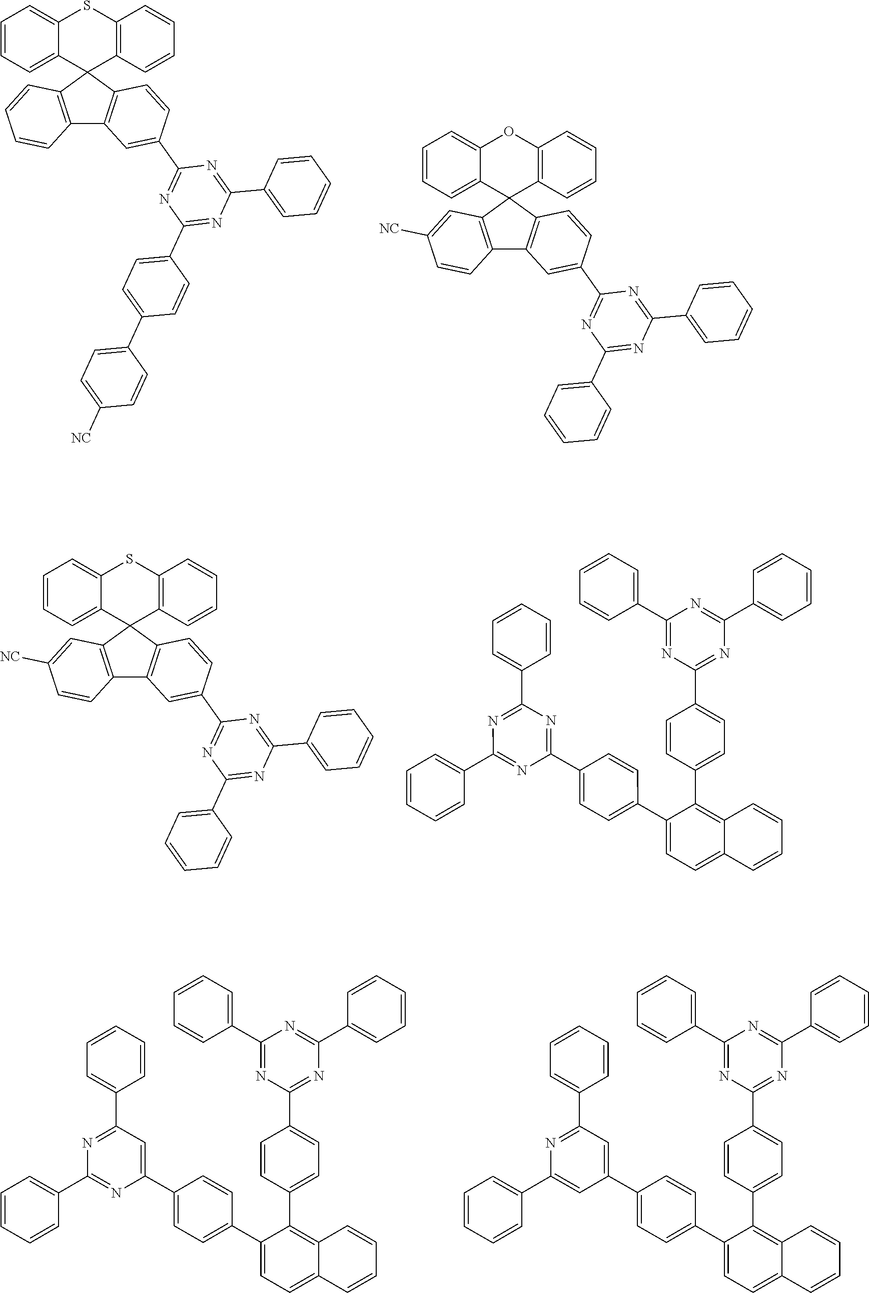

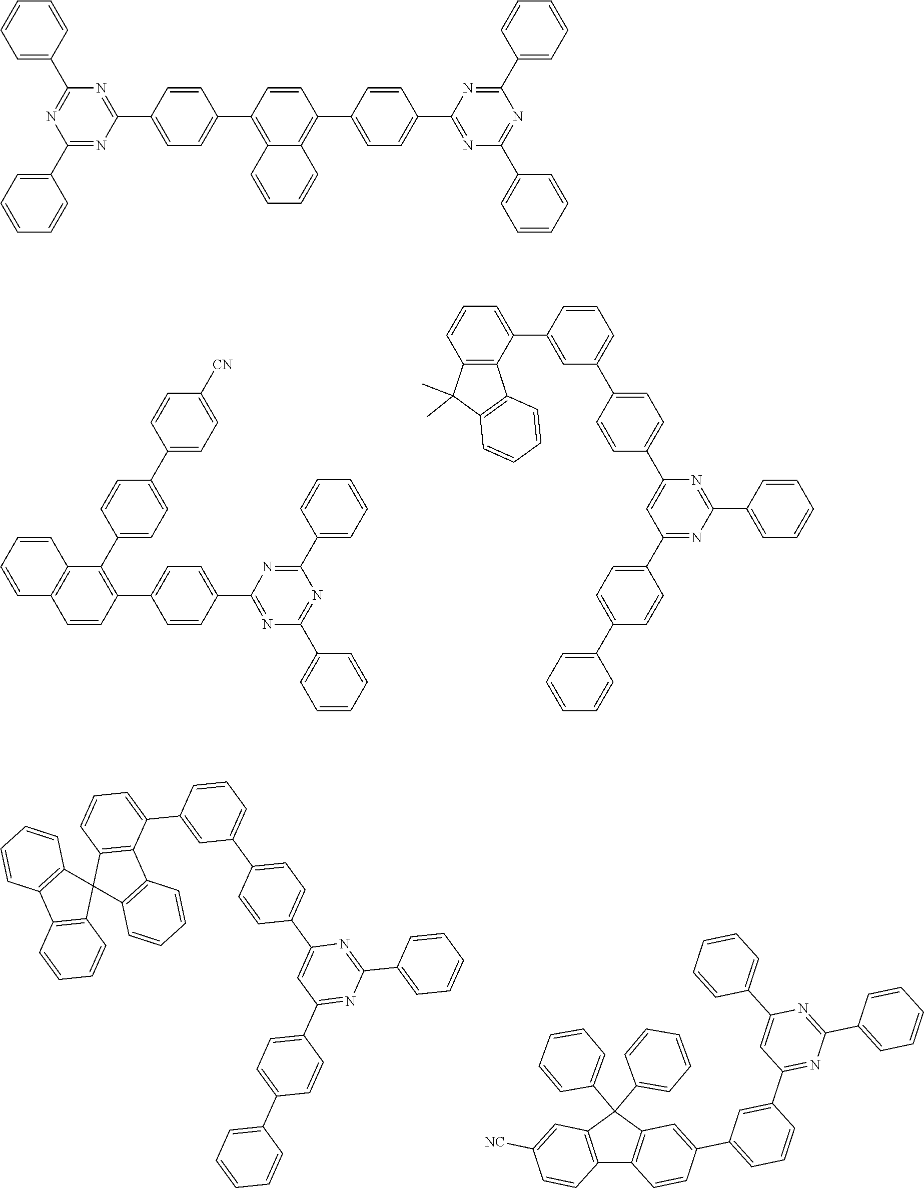 [SMILES: c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1 ; N#Cc1ccc(-c2ccc(-c3c(-c4ccc(-c5nc(-c6ccccc6)nc(-c6ccccc6)n5)cc4)ccc4ccccc34)cc2)cc1 ; CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2]

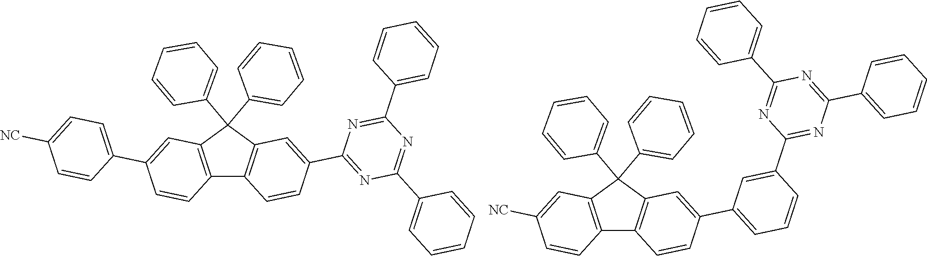

Some example embodiments may provide an organic light emitting diode including the light emitting layer as the organic layer.

Some example embodiments may provide an organic light emitting diode including a light emitting layer and a hole transport region as the organic layer.

Some example embodiments may provide an organic light emitting diode including a light emitting layer and an electron transport region as the organic layer.

Some example embodiments may provide an organic light emitting diode including a hole transport region 140 and an electron transport region 150 in addition to the light emitting layer 130 as the organic layer 105, as shown in the FIGURE.

In an implementation, an organic light emitting diode may further include an electron injection layer, a hole injection layer, or the like, in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The aforementioned organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by suitable methods.

### (Synthesis of Compound for Organic Optoelectronic Device)

### Synthesis Example 1: Synthesis of Intermediate I-1

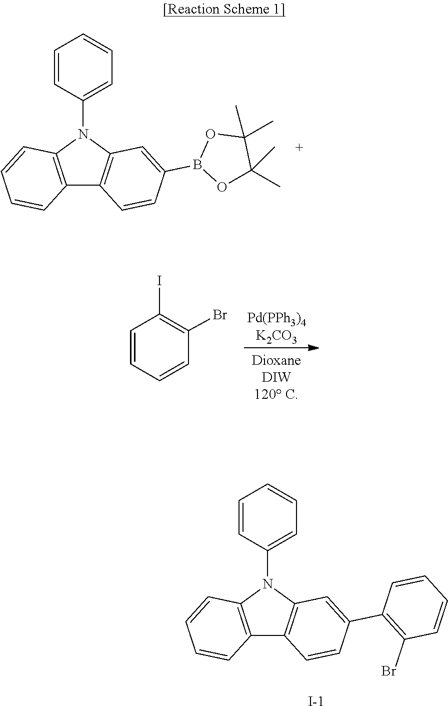 [SMILES: CC1(C)OB(c2ccc3c4ccccc4n(-c4ccccc4)c3c2)OC1(C)C ; Brc1ccccc1I ; Brc1ccccc1-c1ccc2c3ccccc3n(-c3ccccc3)c2c1 ; [I-]]

In a nitrogen environment, after dissolving 9-phenyl-2-(4,4,5,5-tetramethyl-1,3,2-dioxaborolan-2-yl)-9H-carbazole (50 g, 135.4 mmol) in 0.5 L of dioxane purchased from Ukseung Chemical Co. Ltd. (http://www.ukseung.co.kr/), 1-bromo-2-iodobenzene (57.5 g, 203 mmol) purchased from Sigma Aldrich Co., Ltd. (http://www.sigmaaldrich.com/) and tetrakis(triphenylphosphine)palladium (3.1 g, 2.7 mmol) were added thereto and then, stirred. Subsequently, potassium carbonate (46.8 g, 339 mmol) saturated in water was added thereto and then, refluxed by heating at 120° C. for 1 hour. After a reaction was completed, water was added to the reaction solution and then filtered. The obtained residue was separated and purified through flash column chromatography to obtain Intermediate I-1 (37 g, 69%).

HRMS (70 eV, EI+): m/z calcd for C24H16BrN: 397.0466, found: 397.

Elemental Analysis: C, 72%; H, 4%

### Synthesis Example 2: Synthesis of Compound 2

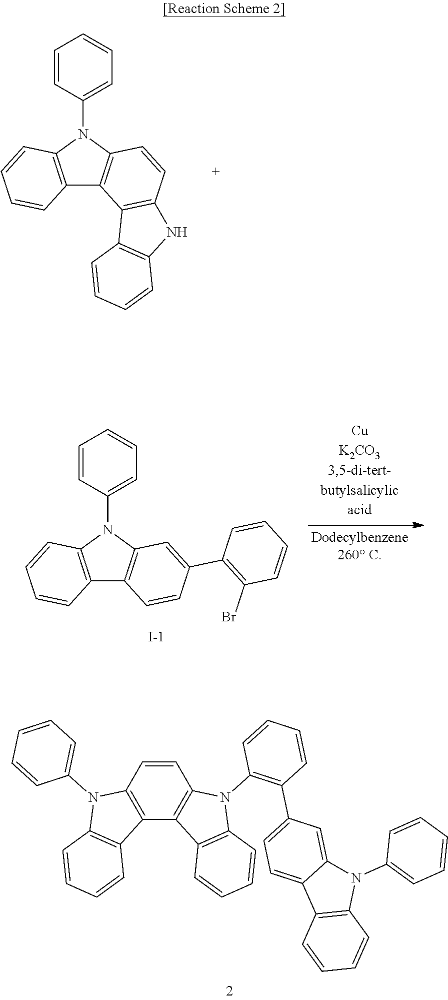 [SMILES: c1ccc(-n2c3ccccc3c3c4c(ccc32)[nH]c2ccccc24)cc1 ; Brc1ccccc1-c1ccc2c3ccccc3n(-c3ccccc3)c2c1 ; [I-] ; c1ccc(-n2c3ccccc3c3ccc(-c4ccccc4-n4c5ccccc5c5c6c7ccccc7n(-c7ccccc7)c6ccc54)cc32)cc1]

In a nitrogen environment, after dissolving 5-phenyl-5,8-dihydroindolo[2,3-c]carbazole (19 g, 57.2 mmol) in 0.2 L of dodecylbenzene purchased from Ukseung Chemical Co. Ltd. (http://www.ukseung.co.kr/), Intermediate I-1 (27.3 g, 68.6 mmol), copper (0.73 g, 11.4 mmol), potassium carbonate (11.8 g, 86 mmol), and 3,5-di-tert-butylsalicylic acid (2.9 g, 11.4 mmol) were added thereto and then, heated at 260° C. for 40 hours. After a reaction was completed, and after adding water to the reaction solution, the mixture was extracted with dichloromethane (DCM), treated with magnesium sulfate anhydrous to remove moisture, filtered, and concentrated under a reduced pressure. The obtained residue was separated and purified through flash column chromatography to obtain Compound 2 (31.6 g, 85%).

HRMS (70 eV, EI+): m/z calcd for C48H₃₁N₃: 649.2518, found: 649.

Elemental Analysis: C, 89%; H, 5%

### Synthesis Example 3: Synthesis of Intermediate I-2

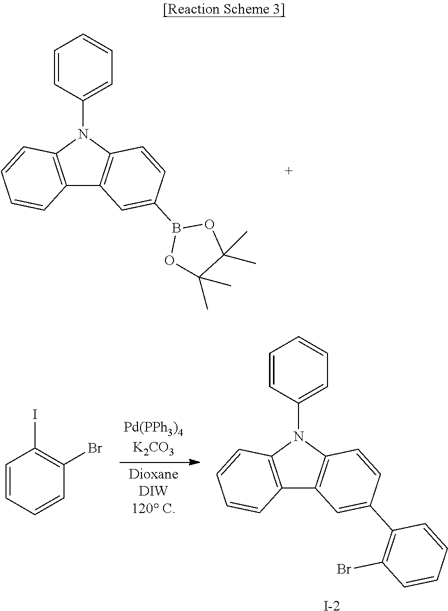

Intermediate I-2 (38.3 g, 71%) was obtained in the same manner as in Synthesis Example 1 except for using 9-phenyl-3-(4,4,5,5-tetramethyl-1,3,2-dioxaborolan-2-yl)-9H-carbazole (50 g, 135.4 mmol) purchased from Ukseung Co., Ltd. (http://www.ukseung.co.kr/) and 1-bromo-2-iodobenzene (57.5 g, 203 mmol) purchased from Sigma Aldrich Co., Ltd. (http://www.sigmaaldrich.com/).

HRMS (70 eV, EI+): m/z calcd for C24H16BrN: 397.0466, found: 397.

Elemental Analysis: C, 72%; H, 4%

### Synthesis Example 4: Synthesis of Compound 3

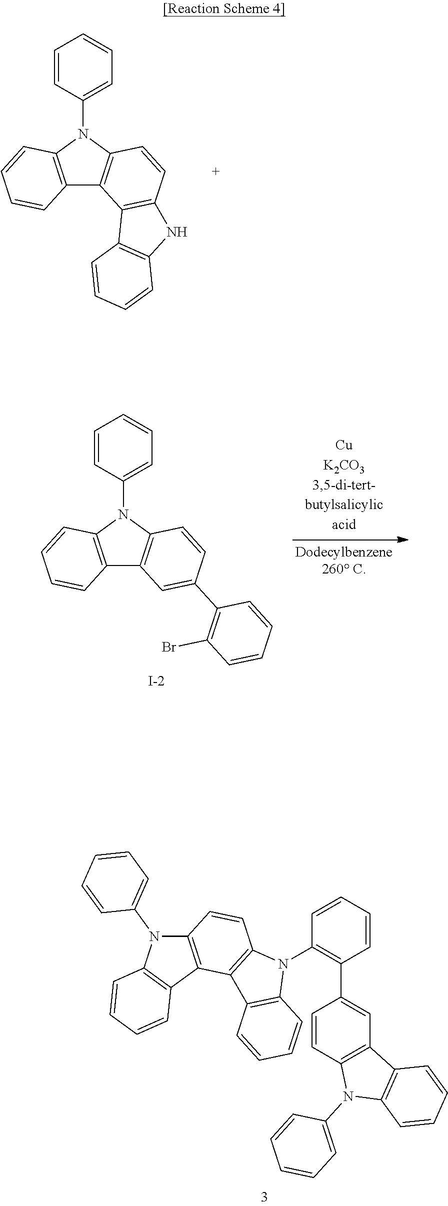

Compound 3 (38.3 g, 71%) was obtained in the same manner as in Synthesis Example 2 except for using 5-phenyl-5,8-dihydroindolo[2,3-c]carbazole (19 g, 57.2 mmol) purchased from Ukseung Co., Ltd. (http://www.ukseung.co.kr/) and Intermediate I-2 (27.3 g, 68.6 mmol).

HRMS (70 eV, EI+): m/z calcd for C48H₃₁N₃: 649.2518, found: 649.

Elemental Analysis: C, 89%; H, 5%

### Synthesis Example 5: Synthesis of Intermediate I-3

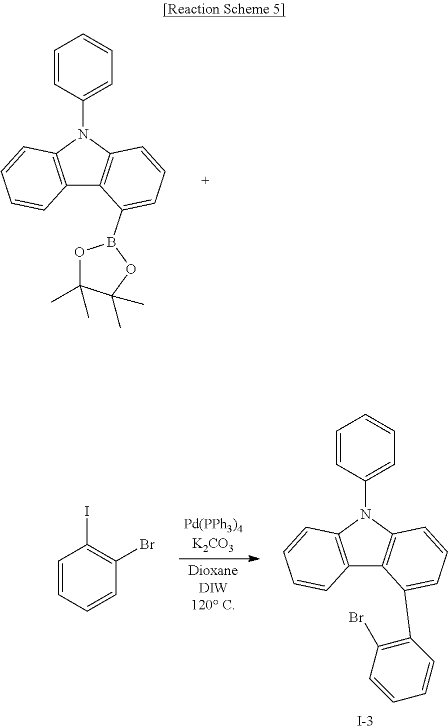

Intermediate I-3 (38.3 g, 71%) was obtained in the same manner as in Synthesis Example 1 except for using 9-phenyl-4-(4,4,5,5-tetramethyl-1,3,2-dioxaborolan-2-yl)-9H-carbazole (50 g, 135.4 mmol) purchased from Ukseung Co., Ltd. (http://www.ukseung.co.kr/) and 1-bromo-2-iodobenzene (57.5 g, 203 mmol) purchased from Sigma Aldrich Co., Ltd. (http://www.sigmaaldrich.com/).

HRMS (70 eV, EI+): m/z calcd for C24H16BrN: 397.0466, found: 397.

Elemental Analysis: C, 72%; H, 4%

### Synthesis Example 6: Synthesis of Compound 4

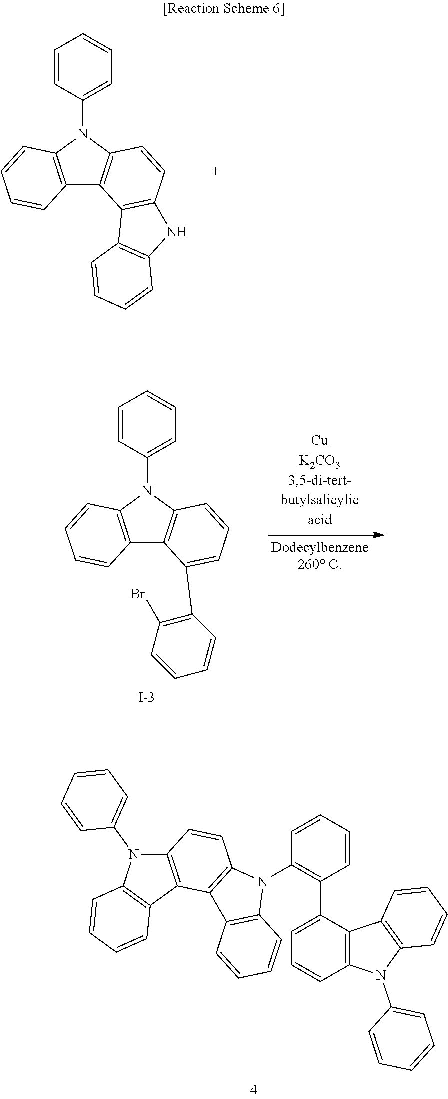

Compound 4 (38.3 g, 71%) was obtained in the same manner as in Synthesis Example 2 except for using 5-phenyl-5,8-dihydroindolo[2,3-c]carbazole (19 g, 57.2 mmol) purchased from Ukseung Co., Ltd. (http://www.ukseung.co.kr/) and Intermediate I-3 (27.3 g, 68.6 mmol).

HRMS (70 eV, EI+): m/z calcd for C48H₃₁N₃: 649.2518, found: 649.

Elemental Analysis: C, 89%; H, 5%

### Synthesis Example 7: Synthesis of Compound 15

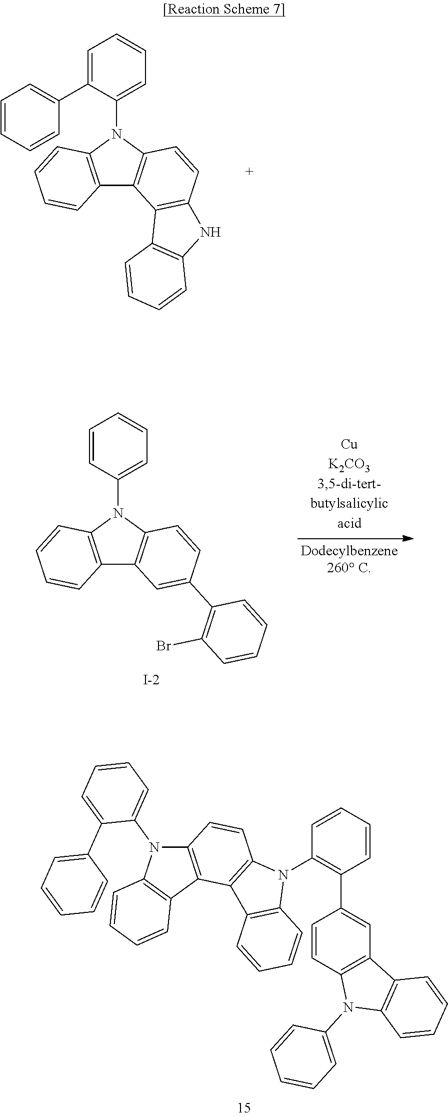

Compound 15 (26.5 g, 64%) was obtained in the same manner as in Synthesis Example 2 except for using 5-([1,1′-biphenyl]-2-yl)-5,8-dihydroindolo[2,3-c]carbazole (23.4 g, 57.2 mmol) purchased from Yantai Gem Chemicals Co., Ltd. (http://www.ytgemchem.com) and Intermediate I-2 (27.3 g, 68.6 mmol).

HRMS (70 eV, EI+): m/z calcd for C54H₃₅N₃: 725.2831, found: 725.

Elemental Analysis: C, 89%; H, 5%

### Synthesis Example 8: Synthesis of Intermediate I-4

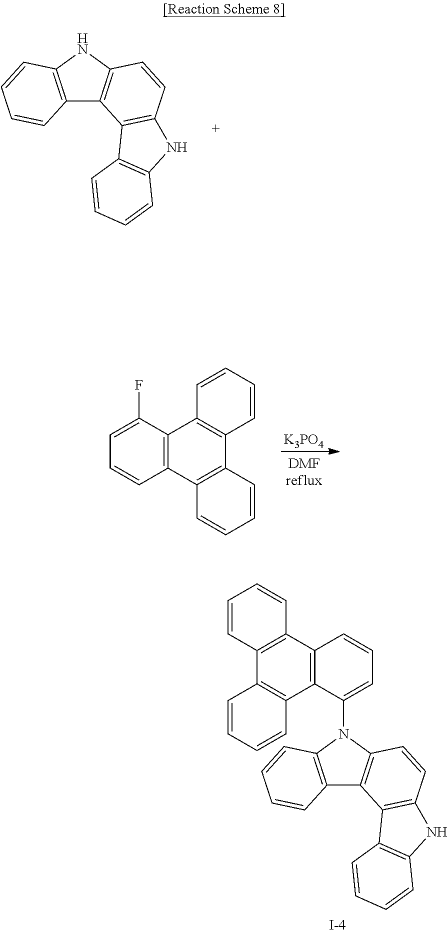

In a nitrogen environment, after dissolving 5,8-dihydroindolo[2,3-c]carbazole (50 g, 195 mmol) purchased from P&H Tech Co., Ltd. (http://www.phtech.co.kr/) and 1-fluorotriphenylene (48 g, 195 mmol) purchased from Yantai Gem Chemicals Co., Ltd. (http://www.ytgemchem.com) in 0.5 L of dimethylforamide (DMF), potassium phosphate tribasic (41.4 g, 195 mmol) was added thereto and then, heated under reflux for 18 hours.

After a reaction was completed, and after removing the solvent through distillation and adding water to the reaction solution, the mixture was extracted with dichloromethane (DCM), treated with magnesium sulfate anhydrous to remove moisture, filtered, and concentrated under a reduced pressure. The obtained residue was separated and purified through flash column chromatography to obtain Intermediate I-4 (37.6 g, 40%).

HRMS (70 eV, EI+): m/z calcd for C36H22N2: 482.1783, found: 482.

Elemental Analysis: C, 90%; H, 5%

### Synthesis Example 9: Synthesis of Compound 190

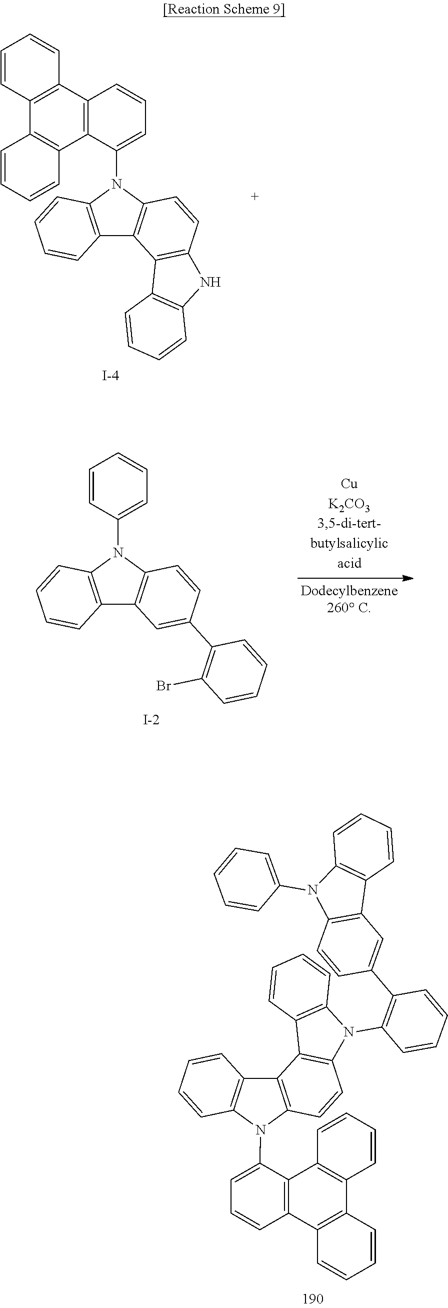

Compound 190 (26.5 g, 61%) was obtained in the same manner as in Synthesis Example 2 except for using Intermediate I-4 (20 g, 41.4 mmol) and Intermediate I-2 (20.2 g, 49.7 mmol).

HRMS (70 eV, EI+): m/z calcd for C90H37N3: 799.2987, found: 799.

Elemental Analysis: C, 90%; H, 5%

### Synthesis Example 10: Synthesis of Intermediate I-5

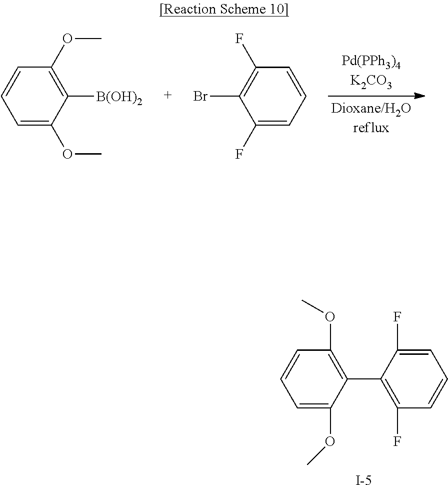

Intermediate I-5 (125 g, 91%) was obtained in the same manner as in Synthesis Example 1 except for using 2,6-dimethoxyphenylboronic acid (100 g, 550 mmol) and 2-bromo-1,3-difluorobenzene (106 g, 550 mmol).

HRMS (70 eV, EI+): m/z calcd for C14H12F₂O₂: 250.0805, found: 250.

Elemental Analysis: C, 67%; H, 5%

### Synthesis Example 11: Synthesis of Intermediate I-6

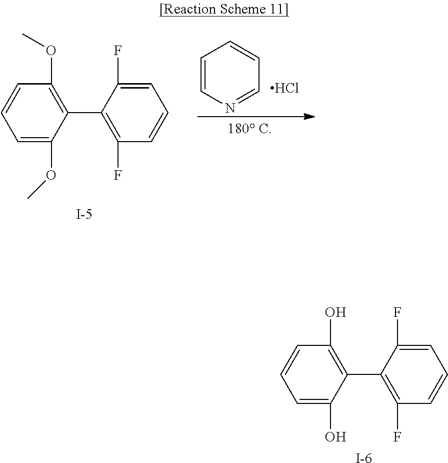

In a nitrogen environment, Intermediate I-5 (121 g, 486 mmol) and pyridine hydrochloride (562 g, 4,861 mmol) were added and then refluxed by heating at 180° C. for 1 hour. After a reaction was completed, and after adding water to the reaction solution, the mixture was extracted with ethyl acetate (EA), treated with magnesium sulfate anhydrous to remove moisture, filtered, and concentrated under a reduced pressure. The obtained residue was separated and purified through flash column chromatography to obtain Intermediate I-6 (102 g, 95%).

HRMS (70 eV, EI+): m/z calcd for C12H8F₂O₂: 222.0492, found: 222.

Elemental Analysis: C, 65%; H, 4%

### Synthesis Example 12: Synthesis of Intermediate I-7

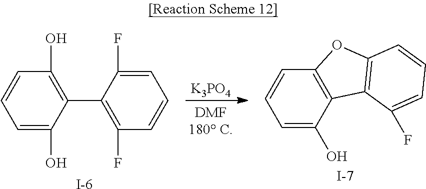

Intermediate I-7 (72.5 g, 80%) was obtained in the same manner as in Synthesis Example 8 except for using Intermediate I-6 (99.5 g, 448 mmol).

HRMS (70 eV, EI+): m/z calcd for C12H7FO₂: 202.0430, found: 202.

Elemental Analysis: C, 71%; H, 3%

### Synthesis Example 13: Synthesis of Intermediate I-8

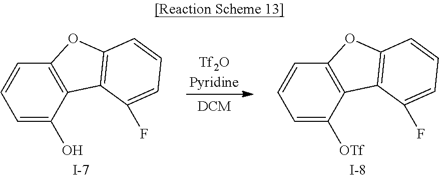

In a nitrogen environment, Intermediate I-7 (72 g, 356 mmol) was dissolved in 0.1 L of dichloromethane (DCM) and then cooled to 0° C. Subsequently, pyridine (120 g, 427 mmol) was added thereto and then, stirred for 30 minutes, and tifluoromethane sulfonic anhydride (33.8 g, 427 mmol) was slowly added thereto and then stirred. After 3 hours, the reaction solution was cooled to 0° C., and after slowly adding water thereto for 30 minutes, the mixture was extracted with dichloromethane (DCM), treated with magnesium sulfate anhydrous to remove moisture, filtered, and concentrated under a reduced pressure. This obtained residue was separated and purified through flash column chromatography to obtain Intermediate I-8 (116 g, 98%).

HRMS (70 eV, EI+): m/z calcd for C13H6F4O4S: 333.9923, found: 333.

Elemental Analysis: C, 47%; H, 2%

### Synthesis Example 14: Synthesis of Intermediate I-9

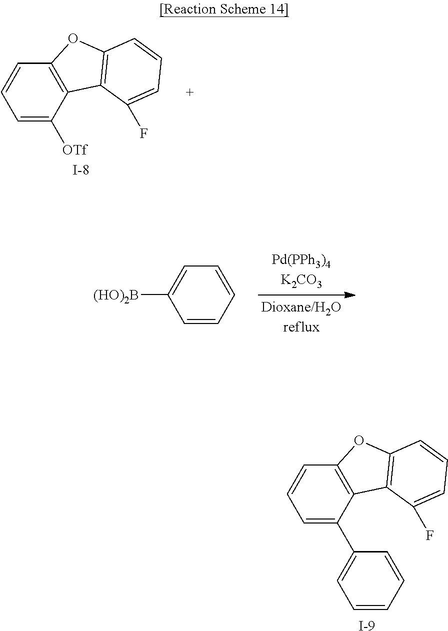

Intermediate I-9 (69.4 g, 78%) was obtained in the same manner as in Synthesis Example 1 except for using Intermediate I-8 (113 g, 339 mmol) and phenylboronic acid (45.5 g, 373 mmol) purchased from Tokyo Chemical Industry Co., Ltd.

HRMS (70 eV, EI+): m/z calcd for C18H11FO: 262.0794, found: 262.

Elemental Analysis: C, 82%; H, 4%

### Synthesis Example 15: Synthesis of Intermediate I-10

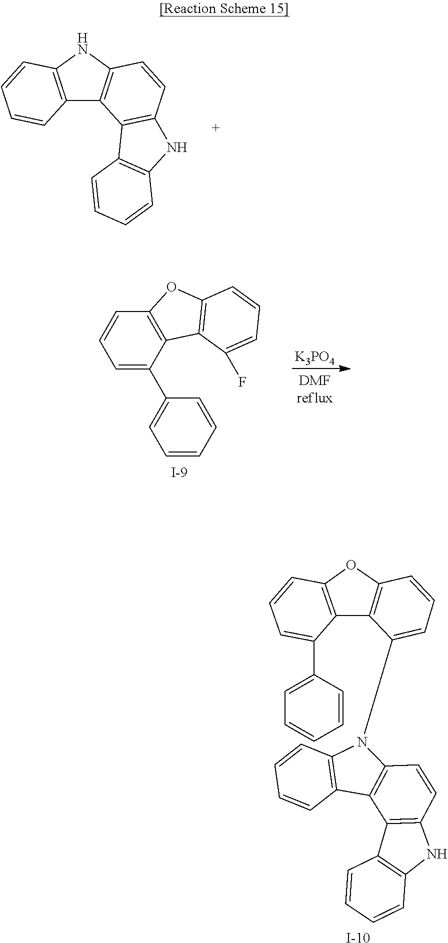

Intermediate I-10 (43.8 g, 45%) was obtained in the same manner as in Synthesis Example 2 except for using 5,8-dihydroindolo[2,3-c]carbazole (50 g, 195 mmol) purchased from P&H Tech Co., Ltd. (http://www.phtech.co.kr/) and Intermediate I-9 (51 g, 195 mmol).

HRMS (70 eV, EI+): m/z calcd for C36H22N₂O: 498.1732, found: 498.

Elemental Analysis: C, 87%; H, 4%

### Synthesis Example 16: Synthesis of Compound 240

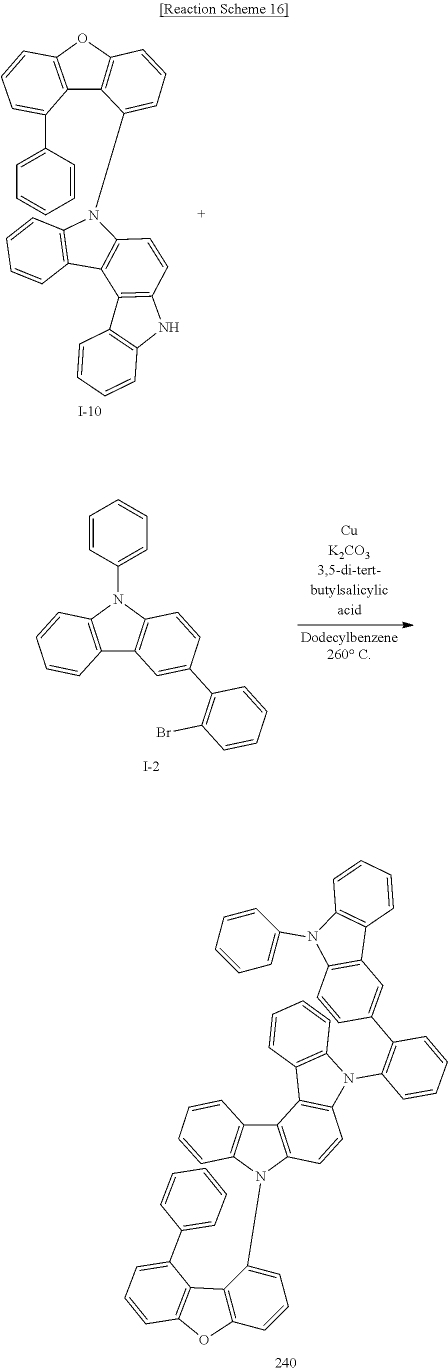

Compound 240 (19 g, 58%) was obtained in the same manner as in Synthesis Example 8 except for using Intermediate I-10 (20 g, 40.1 mmol) and Intermediate I-2 (19.1 g, 48.1 mmol).

HRMS (70 eV, EI+): m/z calcd for C60H37N3O: 815.2937, found: 815.

Elemental Analysis: C, 88%; H, 5%

### Synthesis Example 17: Synthesis of Compound R-1

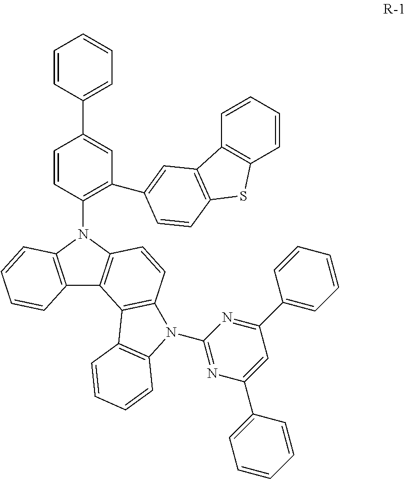 [SMILES: c1ccc(-c2ccc(-n3c4ccccc4c4c5c6ccccc6n(-c6nc(-c7ccccc7)cc(-c7ccccc7)n6)c5ccc43)c(-c3ccc4sc5ccccc5c4c3)c2)cc1]

Compound R-1 was synthesized by referring to the synthesis method of Chinese Patent No. CN 110776513.

HRMS (70 eV, EI+): m/z calcd for C58H36N4S: 820.2661, found: 820.

Elemental Analysis: C, 85%; H, 4%

### Synthesis Example 18: Synthesis of Compound R-2

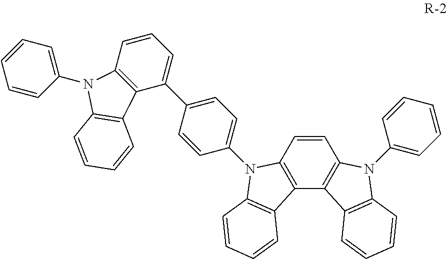 [SMILES: c1ccc(-n2c3ccccc3c3c(-c4ccc(-n5c6ccccc6c6c7c8ccccc8n(-c8ccccc8)c7ccc65)cc4)cccc32)cc1]

Compound R-2 was synthesized by referring to the synthesis method of Korean Patent No. KR 2017-0048094.

HRMS (70 eV, EI+): m/z calcd for C48H31N3: 649.2518, found: 649.

Elemental Analysis: C, 89%; H, 5%

### Synthesis Example 19: Synthesis of Compound R-3

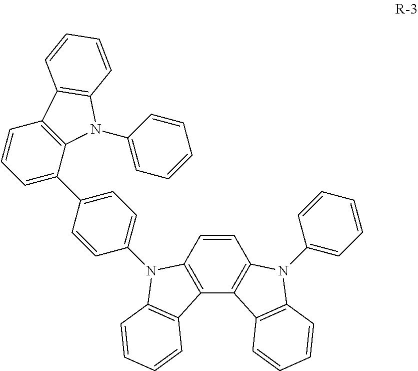 [SMILES: c1ccc(-n2c3ccccc3c3c4c5ccccc5n(-c5ccc(-c6cccc7c8ccccc8n(-c8ccccc8)c67)cc5)c4ccc32)cc1]

Compound R-3 was synthesized by referring to the synthesis method of Korean Patent No. KR 2017-0048094.

HRMS (70 eV, EI+): m/z calcd for C48H31N3: 649.2518, found: 649.

Elemental Analysis: C, 89%; H, 5%

### Synthesis Example 20: Synthesis of Compound R-4

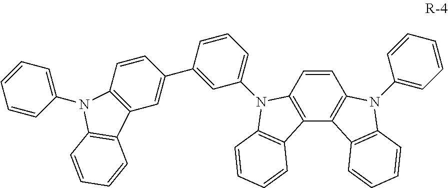 [SMILES: c1ccc(-n2c3ccccc3c3cc(-c4cccc(-n5c6ccccc6c6c7c8ccccc8n(-c8ccccc8)c7ccc65)c4)ccc32)cc1]

Compound R-4 was synthesized by referring to the synthesis method of Korean Patent No. KR 2017-0048094.

HRMS (70 eV, EI+): m/z calcd for C48H31N3: 649.2518, found: 649.

Elemental Analysis: C, 89%; H, 5%

### Synthesis Example 21: Synthesis of Compound R-5

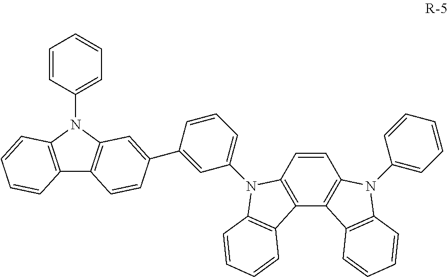 [SMILES: c1ccc(-n2c3ccccc3c3ccc(-c4cccc(-n5c6ccccc6c6c7c8ccccc8n(-c8ccccc8)c7ccc65)c4)cc32)cc1]

Compound R-5 was synthesized by referring to the synthesis method of Korean Patent No. KR 2017-0048094.

HRMS (70 eV, EI+): m/z calcd for C48H31N3: 649.2518, found: 649.

Elemental Analysis: C, 89%; H, 5%

### Synthesis Example 22: Synthesis of Compound E-86

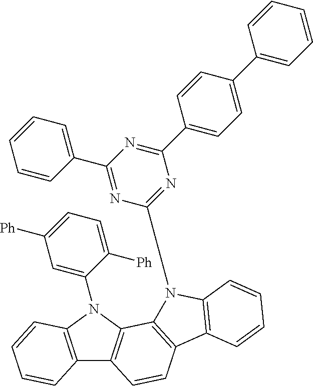 [SMILES: c1ccc(-c2ccc(-c3nc(-c4ccccc4)nc(-n4c5ccccc5c5ccc6c7ccccc7n(-c7cc(-c8ccccc8)ccc7-c7ccccc7)c6c54)n3)cc2)cc1]

Compound E-86 was synthesized by referring to the synthesis method described in Korean Publication No. KR 10-2022-0095942 A.

### Synthesis Example 23: Synthesis of Compound D-33

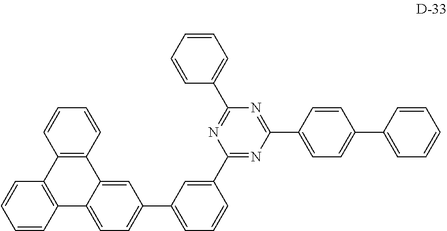 [SMILES: c1ccc(-c2ccc(-c3nc(-c4ccccc4)nc(-c4cccc(-c5ccc6c7ccccc7c7ccccc7c6c5)c4)n3)cc2)cc1]

Compound D-33 was synthesized by referring to the synthesis method described in Korean Registration Publication No. KR 10-1618683 B1.

### Example 1: Manufacturing of Green Organic Light Emitting Diode (Single Host)

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. On the hole transport layer, Compound B was deposited to a thickness of 350 Å to form a hole transport auxiliary layer. On the hole transport auxiliary layer, Compound 2 (synthesized in Synthesis Example 2) was used as a host and 10 wt % of PhGD was doped as a dopant to form a 400 Å-thick light emitting layer by vacuum deposition. Subsequently, on the light emitting layer, Compound C was deposited to form a 50 Å-thick electron transport auxiliary layer, and Compound D and Liq in a weight ratio of 1:1 were simultaneously vacuum-deposited to form a 300 Å-thick electron transport layer. On the electron transport layer, a cathode was formed by sequentially vacuum-depositing 15 Å of LiQ and 1,200 Å of Al, manufacturing an organic light emitting diode.

The organic light emitting diode was manufactured to have a structure of ITO/Compound A (3% NDP-9 doping, 100 Å)/Compound A (1,350 Å)/Compound B (350 Å)/EML [Host (Compound 2): PhGD=90 wt %: 10 wt %](400 Å)/Compound C (50 Å)/Compound D:LiQ (300 Å) LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-Dibenzofuranyl)phenyl]-N-[4-(9-phenyl-9H-fluoren-9-yl)phenyl][1,1′-biphenyl]-4-amine
Compound C: 2,4-Diphenyl-6-(4′,5′,6′-triphenyl[1,1′: 2′,1″: 3″,1′″3′″,1″″-quinquephenyl]-3″″-yl)-1,3,5-triazine
Compound D: 2-(1,1′-Biphenyl-4-yl)-4-(9,9-diphenylfluoren-4-yl)-6-phenyl-1,3,5-triazine

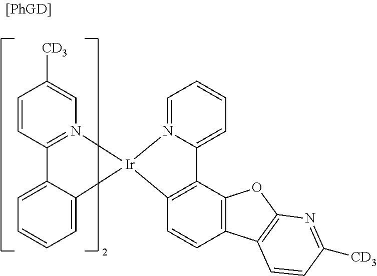

### Examples 2 to 6 and Comparative Examples 1 to 5

Organic light emitting diodes were manufactured in the same manner as Example 1, except that the compositions were changed to the those shown in Table 1.

### Example 7: Manufacturing of Green Organic Light Emitting Diode (Mixed Host)

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. On the hole transport layer, Compound E was deposited to a thickness of 320 Å to form a hole transport auxiliary layer. On the hole transport auxiliary layer, Compound 2 (synthesized in Synthesis Example 2) and Compound E-86 (synthesized in Synthesis Example 22) were simultaneously used as hosts in a weight ratio of 7:3, and PtGD was doped at 15 wt % as a dopant to form a 380 Å-thick light emitting layer by vacuum deposition. Subsequently, on the light emitting layer, Compound F was deposited to a thickness of 50 Å to form an electron transport auxiliary layer, and Compound G and Liq were simultaneously vacuum deposited in a weight ratio of 1:1 to form an electron transport layer with a thickness of 300 Å. On the electron transport layer, a cathode was formed by sequentially vacuum-depositing 15 Å of LiQ and 1,200 Å of Al, manufacturing an organic light emitting diode.

The organic light emitting diode was manufactured to have a structure of ITO/Compound A (3% NDP-9 doping, 100 Å)/Compound A (1,350 Å)/Compound E (320 Å)//EML [Host (Compound 2:Compound E-86=7:3 wt %/wt %): PtGD=85 wt %: 15 wt %]380 Å/Compound F (50 Å)/Compound G:LiQ (300 Å)/LiQ (15 Å)/Al (1,200 Å).
Compound E: N,N-bis(9,9-dimethyl-9H-fluoren-4-yl)-9,9-spirobi(fluorene)-2-amine
Compound F: 2-[3′-(9,9-Dimethyl-9H-fluoren-2-yl)[1,1′-biphenyl]-3-yl]-4,6-diphenyl-1,3,5-triazine
Compound G: 2-[4-[4-(4′-Cyano-1,1′-biphenyl-4-yl)-1-naphthyl]phenyl]-4,6-diphenyl-1,3,5-triazine

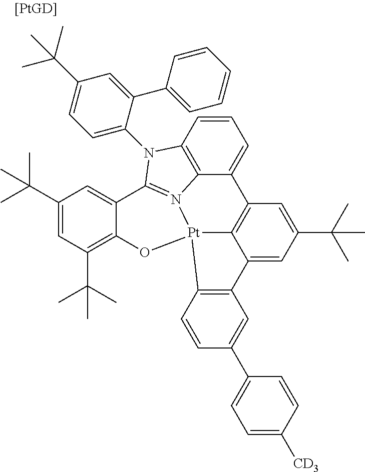

### Example 8 and Example 10

Each organic light emitting diode was manufactured in the same manner as in Example 7, except that the host mixing ratio was changed to a weight ratio of 6:4.

### Example 9 and Examples 11 to 15 and Comparative Examples 6 to 10

Each organic light emitting diode was manufactured in the same manner as Example 7, except that the composition was changed to the those shown in Table 2.

### Evaluation

The luminous efficiency and life-span characteristics of the organic light emitting diodes according to Examples 1 to 15 and Comparative Examples 1 to 10 were evaluated.

The measurement method was as follows, and the results are as shown in Tables 1 and 2.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) were calculated by using the luminance and current density from (1) and (2) above and a voltage.

The luminous efficiency values of Examples 1 to 6 and Comparative Examples 1 to 5 were calculated as relative values based on Comparative Example 1 and are shown in Table 1.

The luminous efficiency values of Examples 7 to 15 and Comparative Examples 6 to 10 were calculated as relative values based on Comparative Example 6 and are shown in Table 2.

### (4) Measurement of Life-Span

The results were obtained by maintaining the luminance (cd/m²) at 24,000 cd/m2 and measuring the time for the current efficiency (cd/A) to decrease to 97%.

The life-span measurements of Examples 1 to 6 and Comparative Examples 1 to 5 were calculated as relative values based on Comparative Example 1 and are shown in Table 1.

The life-span measurements of Examples 7 to 15 and Comparative Examples 6 to 10 were calculated as relative values based on Comparative Example 6 and are shown in Table 2.

TABLE 1

| | | Luminous | |
|---|---|---|---|
| | | efficiency | Life-span |
| No. | Compound | (%) | (%) |
| Example 1 | 2 | 105 | 400 |
| Example 2 | 3 | 110 | 480 |
| Example 3 | 4 | 112 | 430 |
| Example 4 | 15 | 116 | 350 |
| Example 5 | 190 | 115 | 450 |
| Example 6 | 240 | 117 | 550 |
| Comparative | R-1 | 100 | 100 |
| Example 1 | | | |
| Comparative | R-2 | 86 | 300 |
| Example 2 | | | |
| Comparative | R-3 | 93 | 0 |
| Example 3 | | | |
| Comparative | R-4 | 96 | 250 |
| Example 4 | | | |
| Comparative | R-5 | 88 | 200 |
| Example 5 | | | |

TABLE 2

| | Compounds | | | |
|---|---|---|---|---|
| | | Second | Luminous | Life-span |
| No. | First compound | compound | efficiency (%) | (%) |
| Example 7 | 2 | E-86 | 103 | 413 |
| Example 8 | 2 | E-86 | 116 | 575 |
| Example 9 | 2 | D-33 | 122 | 575 |
| Example 10 | 2 | D-33 | 124 | 500 |
| Example 11 | 3 | E-86 | 110 | 475 |
| Example 12 | 4 | E-86 | 116 | 438 |
| Example 13 | 15 | E-86 | 119 | 375 |
| Example 14 | 190 | E-86 | 118 | 463 |
| Example 15 | 240 | E-86 | 122 | 525 |
| Comparative | R-1 | E-86 | 100 | 100 |
| Example 6 | | | | |
| Comparative | R-2 | E-86 | 88 | 250 |
| Example 7 | | | | |
| Comparative | R-3 | E-86 | 96 | 0 |
| Example 8 | | | | |
| Comparative | R-4 | E-86 | 97 | 200 |
| Example 9 | | | | |
| Comparative | R-5 | E-86 | 90 | 150 |
| Example 10 | | | | |

Referring to Tables 1 and 2, the organic light emitting diodes according to Examples 1 to 15 had significantly improved luminous efficiency and life-span characteristics, compared to the organic light emitting diodes according to Comparative Examples 1 to 10.

One or more embodiments may provide a compound for an organic optoelectronic device capable of lowering a driving voltage, and implementing an organic optoelectronic device with high efficiency and a long life-span.

It is possible to implement high-efficiency, long life-span organic optoelectronic devices while lowering the driving voltage.

Example embodiments have been disclosed herein, and although specific terms are employed, they are used and are to be interpreted in a generic and descriptive sense only and not for purposes of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 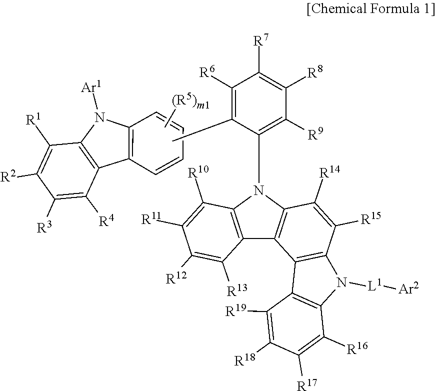
wherein, in Chemical Formula 1,
L¹ is a single bond, a substituted or unsubstituted C6 to C30 arylene group, or a substituted or unsubstituted C2 to C30 heteroarylene group,
Ar¹ and Ar² are each independently a substituted or unsubstituted C6 to C30 aryl group or a substituted or unsubstituted C2 to C30 heterocyclic group,
R¹ to R¹⁹ are each independently hydrogen, deuterium, a cyano group, a halogen, a substituted or unsubstituted C1 to C30 alkyl group, a substituted or unsubstituted C6 to C30 aryl group, a substituted or unsubstituted C2 to C30 heterocyclic group, a substituted or unsubstituted C1 to C10 alkylsilyl group, or a combination thereof,
m1 is an integer of 1 to 3, and
when m1 is 2 or 3, each R⁵ is the same or different from each other.

2. The compound for an organic optoelectronic device as claimed in claim 1, wherein:
Chemical Formula 1 is represented by one of Chemical Formula 1-1 to Chemical Formula 1-4: 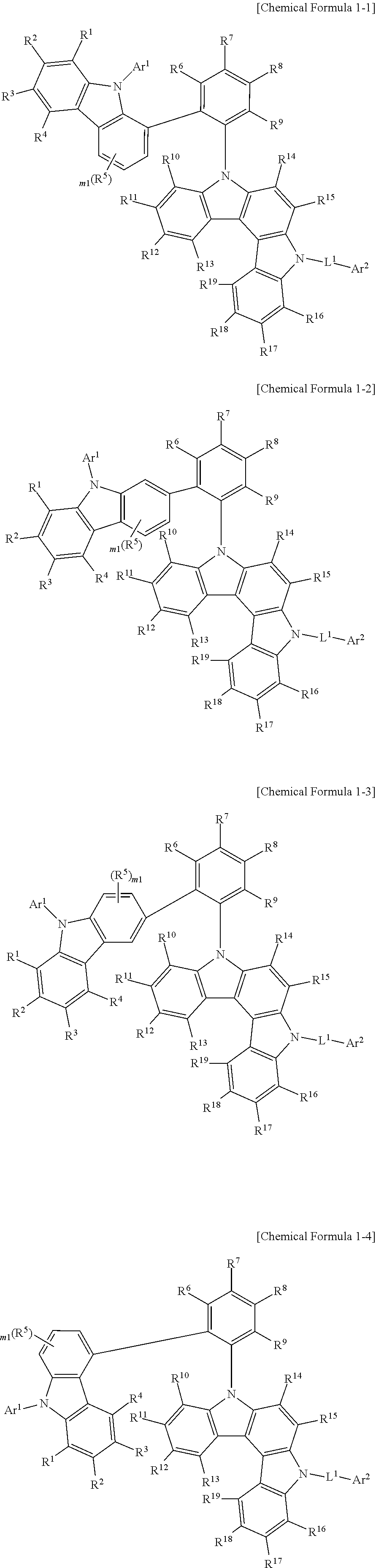
in Chemical Formula 1-1 to Chemical Formula 1-4, L¹, Ar¹ and Ar², R¹ to R¹⁹, and m1 are defined the same as those of Chemical Formula 1.

3. The compound for an organic optoelectronic device as claimed in claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted triphenylene group, a substituted or unsubstituted dibenzofuranyl group, a substituted or unsubstituted dibenzothiophenyl group, or a substituted or unsubstituted dibenzosilolyl group.

4. The compound for an organic optoelectronic device as claimed in claim 1, wherein:
moieties *L¹-Ar² and *—Ar¹ are each independently a moiety of Group I: 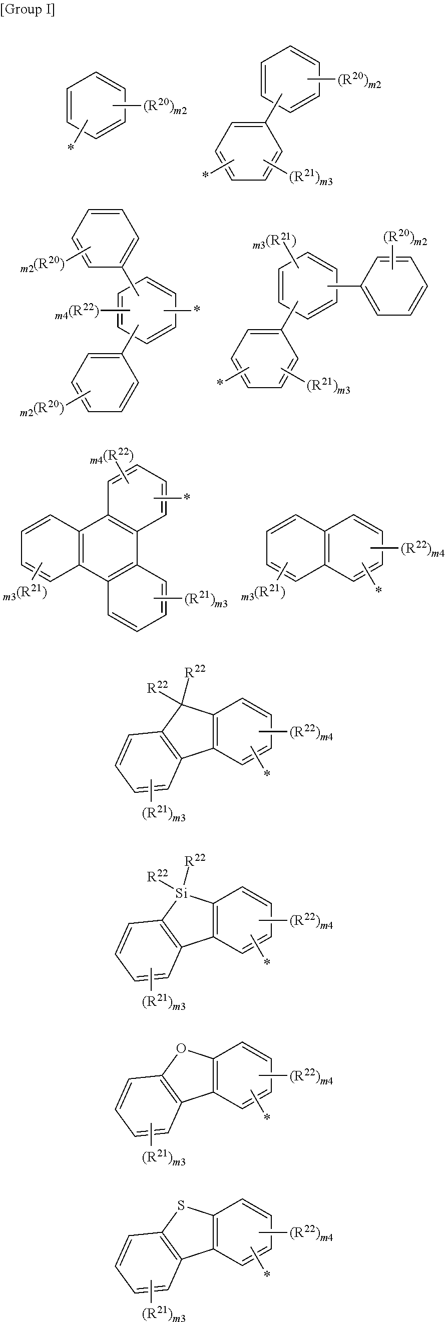
in Group I,
R²⁰ to R²² are each independently hydrogen, deuterium, a cyano group, a substituted or unsubstituted C1 to C10 alkyl group, or a substituted or unsubstituted C6 to C12 aryl group,
m2 is an integer of 1 to 5,
m3 is an integer of 1 to 4,
m4 is an integer of 1 to 3, and
* is a linking point.

5. The compound for an organic optoelectronic device as claimed in claim 1, wherein R¹ to R¹⁹ are each independently hydrogen, deuterium, a cyano group, a substituted or unsubstituted C1 to C10 alkyl group, a substituted or unsubstituted C6 to C12 aryl group, a substituted or unsubstituted C2 to C20 heterocyclic group, a substituted or unsubstituted C1 to C10 alkylsilyl group, or a combination thereof.

6. The compound for an organic optoelectronic device as claimed in claim 1, wherein:
the compound is a compound of Group 1: 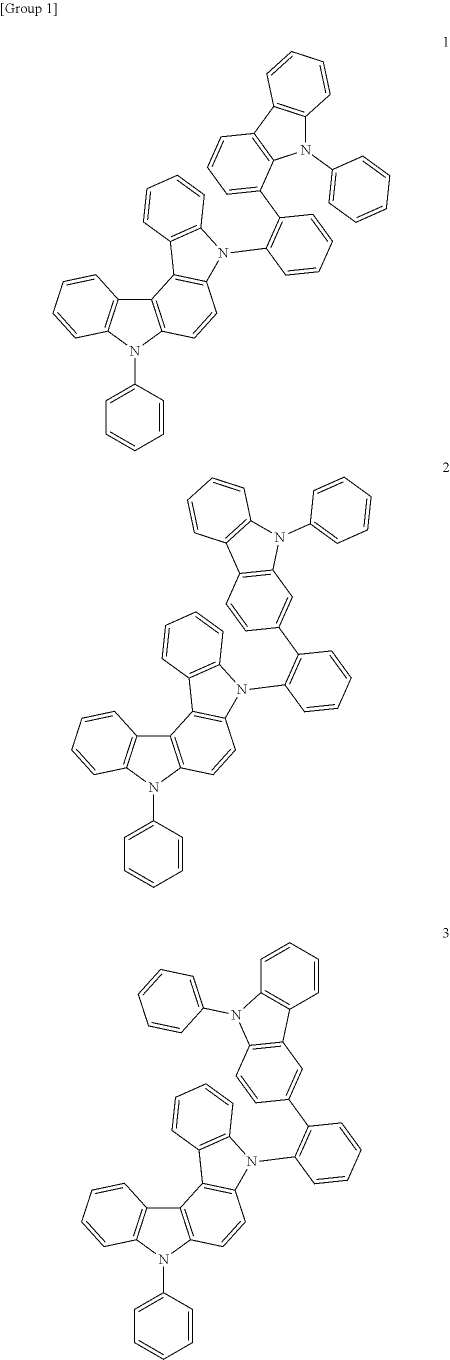 [SMILES: c1ccc(-n2c3ccccc3c3c4c5ccccc5n(-c5ccccc5-c5cccc6c7ccccc7n(-c7ccccc7)c56)c4ccc32)cc1 ; c1ccc(-n2c3ccccc3c3ccc(-c4ccccc4-n4c5ccccc5c5c6c7ccccc7n(-c7ccccc7)c6ccc54)cc32)cc1 ; c1ccc(-n2c3ccccc3c3cc(-c4ccccc4-n4c5ccccc5c5c6c7ccccc7n(-c7ccccc7)c6ccc54)ccc32)cc1] 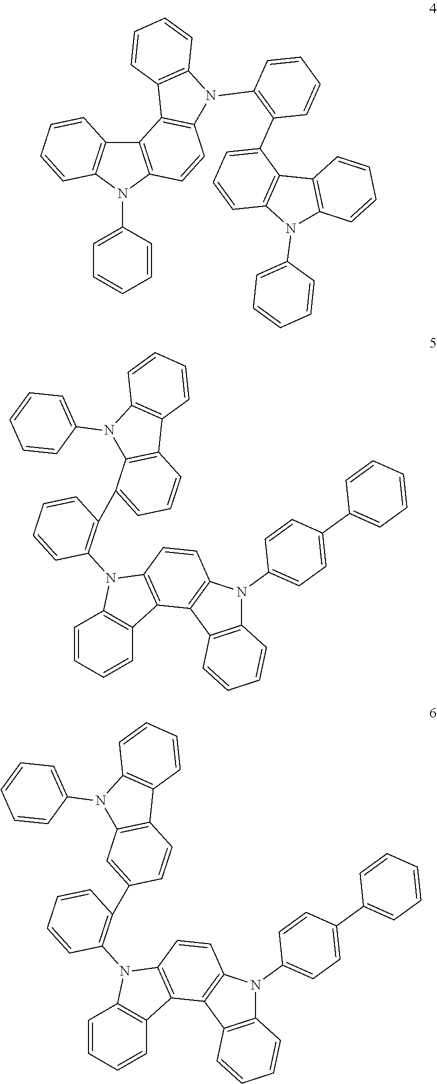 [SMILES: c1ccc(-n2c3ccccc3c3c(-c4ccccc4-n4c5ccccc5c5c6c7ccccc7n(-c7ccccc7)c6ccc54)cccc32)cc1 ; c1ccc(-c2ccc(-n3c4ccccc4c4c5c6ccccc6n(-c6ccccc6-c6cccc7c8ccccc8n(-c8ccccc8)c67)c5ccc43)cc2)cc1 ; c1ccc(-c2ccc(-n3c4ccccc4c4c5c6ccccc6n(-c6ccccc6-c6ccc7c8ccccc8n(-c8ccccc8)c7c6)c5ccc43)cc2)cc1] 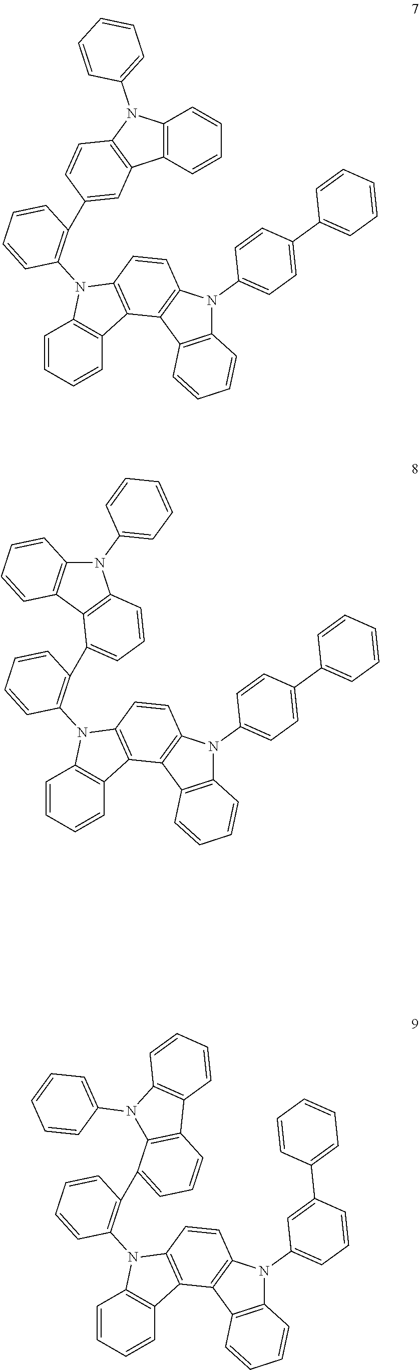 [SMILES: c1ccc(-c2ccc(-n3c4ccccc4c4c5c6ccccc6n(-c6ccccc6-c6ccc7c(c6)c6ccccc6n7-c6ccccc6)c5ccc43)cc2)cc1 ; c1ccc(-c2ccc(-n3c4ccccc4c4c5c6ccccc6n(-c6ccccc6-c6cccc7c6c6ccccc6n7-c6ccccc6)c5ccc43)cc2)cc1 ; c1ccc(-c2cccc(-n3c4ccccc4c4c5c6ccccc6n(-c6ccccc6-c6cccc7c8ccccc8n(-c8ccccc8)c67)c5ccc43)c2)cc1] 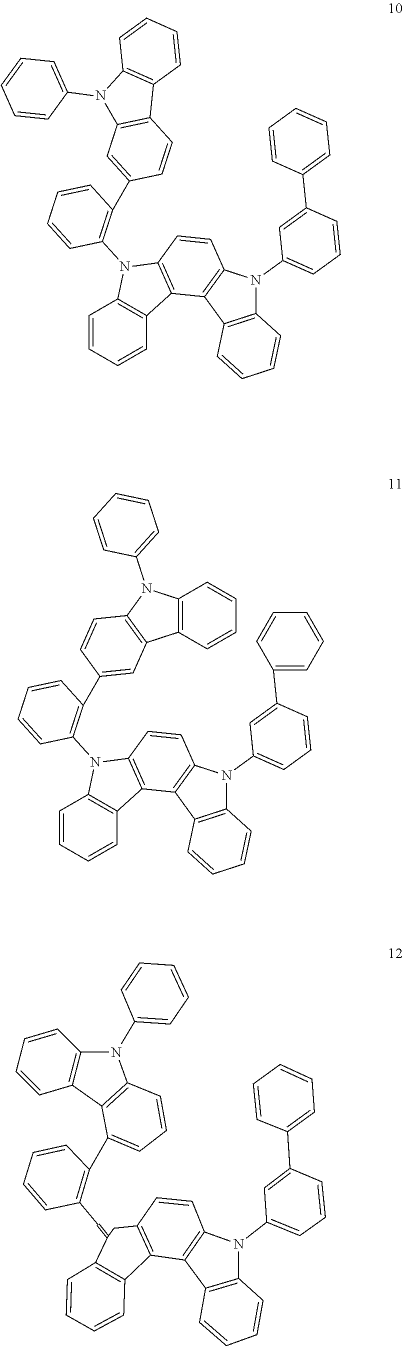 [SMILES: c1ccc(-c2cccc(-n3c4ccccc4c4c5c6ccccc6n(-c6ccccc6-c6ccc7c8ccccc8n(-c8ccccc8)c7c6)c5ccc43)c2)cc1 ; c1ccc(-c2cccc(-n3c4ccccc4c4c5c6ccccc6n(-c6ccccc6-c6ccc7c(c6)c6ccccc6n7-c6ccccc6)c5ccc43)c2)cc1 ; C(=C1Cc2ccc3c(c2-c2ccccc21)c1ccccc1n3-c1cccc(-c2ccccc2)c1)c1ccccc1-c1cccc2c1c1ccccc1n2-c1ccccc1] 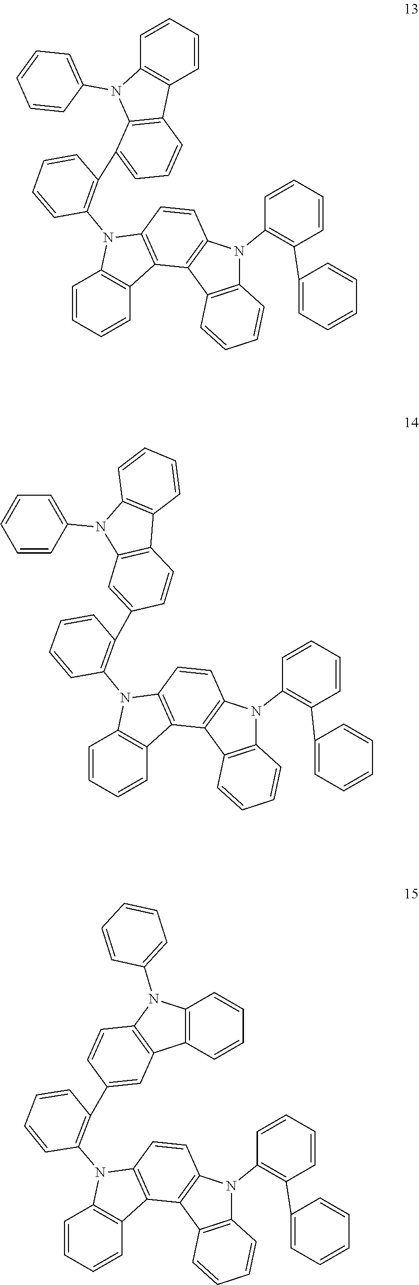 [SMILES: c1ccc(-c2ccccc2-n2c3ccccc3c3c4c5ccccc5n(-c5ccccc5-c5cccc6c7ccccc7n(-c7ccccc7)c56)c4ccc32)cc1 ; c1ccc(-c2ccccc2-n2c3ccccc3c3c4c5ccccc5n(-c5ccccc5-c5ccc6c7ccccc7n(-c7ccccc7)c6c5)c4ccc32)cc1 ; c1ccc(-c2ccccc2-n2c3ccccc3c3c4c5ccccc5n(-c5ccccc5-c5ccc6c(c5)c5ccccc5n6-c5ccccc5)c4ccc32)cc1] 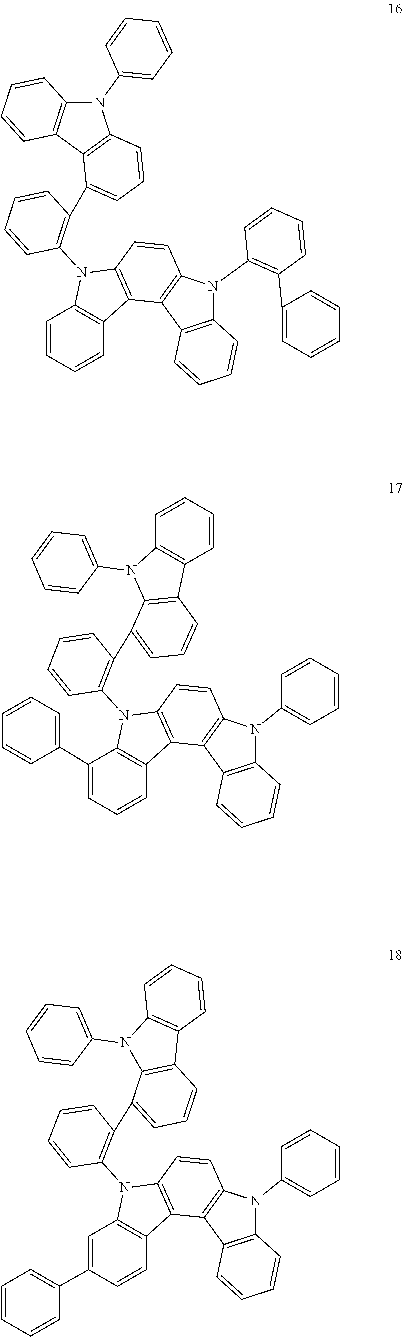 [SMILES: c1ccc(-c2ccccc2-n2c3ccccc3c3c4c5ccccc5n(-c5ccccc5-c5cccc6c5c5ccccc5n6-c5ccccc5)c4ccc32)cc1 ; c1ccc(-c2cccc3c4c5c6ccccc6n(-c6ccccc6)c5ccc4n(-c4ccccc4-c4cccc5c6ccccc6n(-c6ccccc6)c45)c23)cc1 ; c1ccc(-c2ccc3c4c5c6ccccc6n(-c6ccccc6)c5ccc4n(-c4ccccc4-c4cccc5c6ccccc6n(-c6ccccc6)c45)c3c2)cc1] 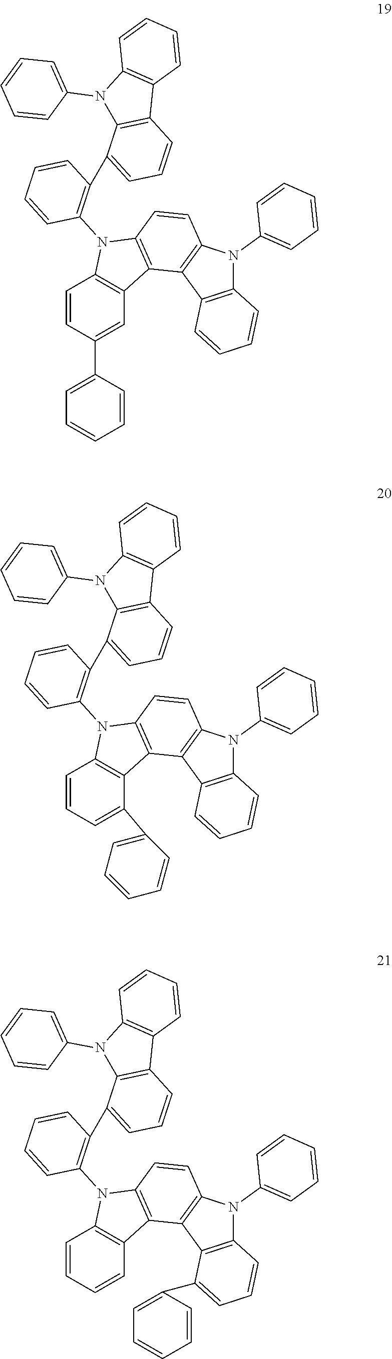 [SMILES: c1ccc(-c2ccc3c(c2)c2c4c5ccccc5n(-c5ccccc5)c4ccc2n3-c2ccccc2-c2cccc3c4ccccc4n(-c4ccccc4)c23)cc1 ; c1ccc(-c2cccc3c2c2c4c5ccccc5n(-c5ccccc5)c4ccc2n3-c2ccccc2-c2cccc3c4ccccc4n(-c4ccccc4)c23)cc1 ; c1ccc(-c2cccc3c2c2c4c5ccccc5n(-c5ccccc5-c5cccc6c7ccccc7n(-c7ccccc7)c56)c4ccc2n3-c2ccccc2)cc1] 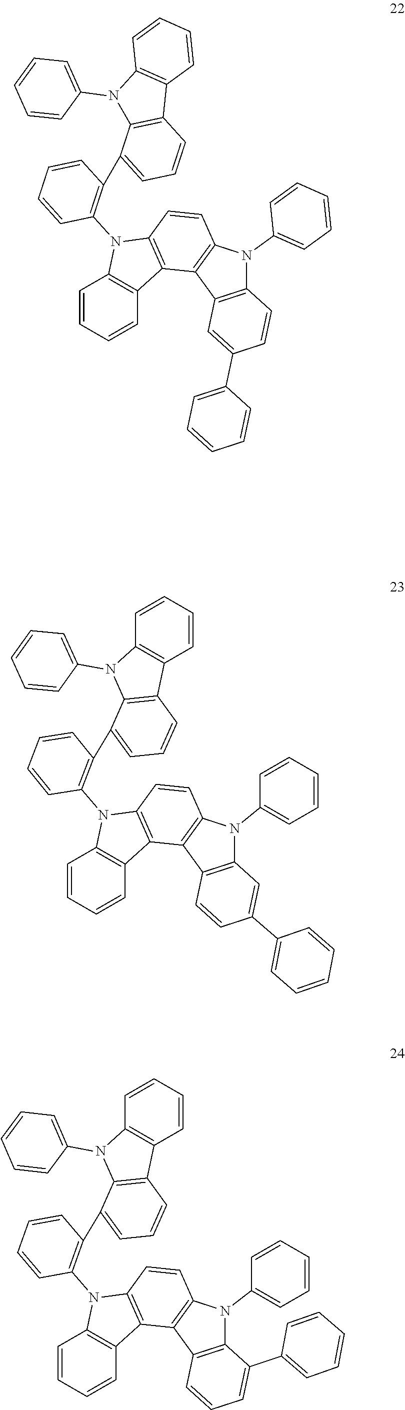 [SMILES: c1ccc(-c2ccc3c(c2)c2c4c5ccccc5n(-c5ccccc5-c5cccc6c7ccccc7n(-c7ccccc7)c56)c4ccc2n3-c2ccccc2)cc1 ; c1ccc(-c2ccc3c4c5c6ccccc6n(-c6ccccc6-c6cccc7c8ccccc8n(-c8ccccc8)c67)c5ccc4n(-c4ccccc4)c3c2)cc1 ; c1ccc(-c2cccc3c4c5c6ccccc6n(-c6ccccc6-c6cccc7c8ccccc8n(-c8ccccc8)c67)c5ccc4n(-c4ccccc4)c23)cc1] 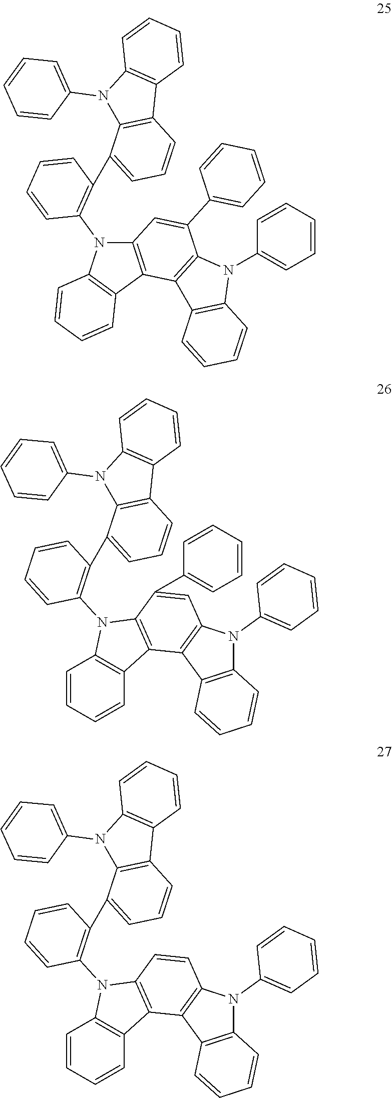 [SMILES: c1ccc(-c2cc3c(c4ccccc4n3-c3ccccc3-c3cccc4c5ccccc5n(-c5ccccc5)c34)c3c4ccccc4n(-c4ccccc4)c23)cc1 ; c1ccc(-c2cc3c(c4ccccc4n3-c3ccccc3)c3c4ccccc4n(-c4ccccc4-c4cccc5c6ccccc6n(-c6ccccc6)c45)c23)cc1 ; c1ccc(-n2c3ccccc3c3c4c5ccccc5n(-c5ccccc5-c5cccc6c7ccccc7n(-c7ccccc7)c56)c4ccc32)cc1] 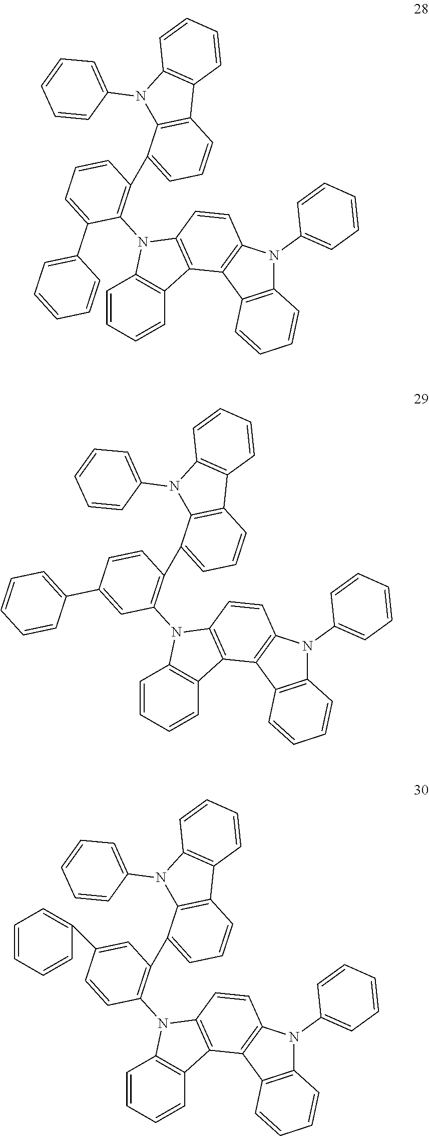 [SMILES: c1ccc(-c2cccc(-c3cccc4c5ccccc5n(-c5ccccc5)c34)c2-n2c3ccccc3c3c4c5ccccc5n(-c5ccccc5)c4ccc32)cc1 ; c1ccc(-c2ccc(-c3cccc4c5ccccc5n(-c5ccccc5)c34)c(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccccc6)c5ccc43)c(-c3cccc4c5ccccc5n(-c5ccccc5)c34)c2)cc1] 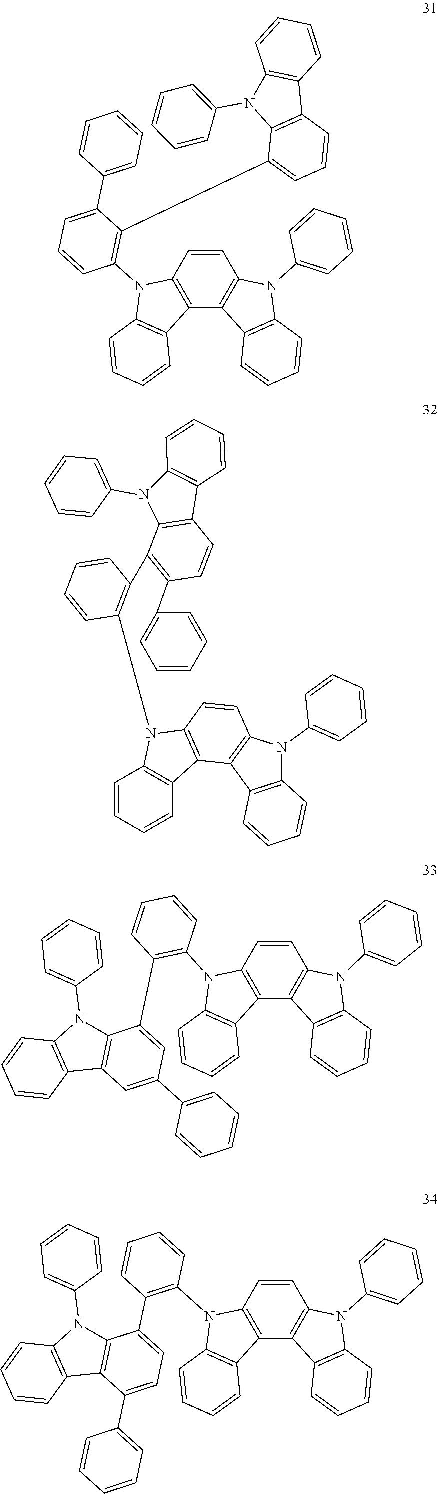 [SMILES: c1ccc(-c2cccc(-n3c4ccccc4c4c5c6ccccc6n(-c6ccccc6)c5ccc43)c2-c2cccc3c4ccccc4n(-c4ccccc4)c23)cc1 ; c1ccc(-c2ccc3c4ccccc4n(-c4ccccc4)c3c2-c2ccccc2-n2c3ccccc3c3c4c5ccccc5n(-c5ccccc5)c4ccc32)cc1 ; c1ccc(-c2cc(-c3ccccc3-n3c4ccccc4c4c5c6ccccc6n(-c6ccccc6)c5ccc43)c3c(c2)c2ccccc2n3-c2ccccc2)cc1 ; c1ccc(-c2ccc(-c3ccccc3-n3c4ccccc4c4c5c6ccccc6n(-c6ccccc6)c5ccc43)c3c2c2ccccc2n3-c2ccccc2)cc1] 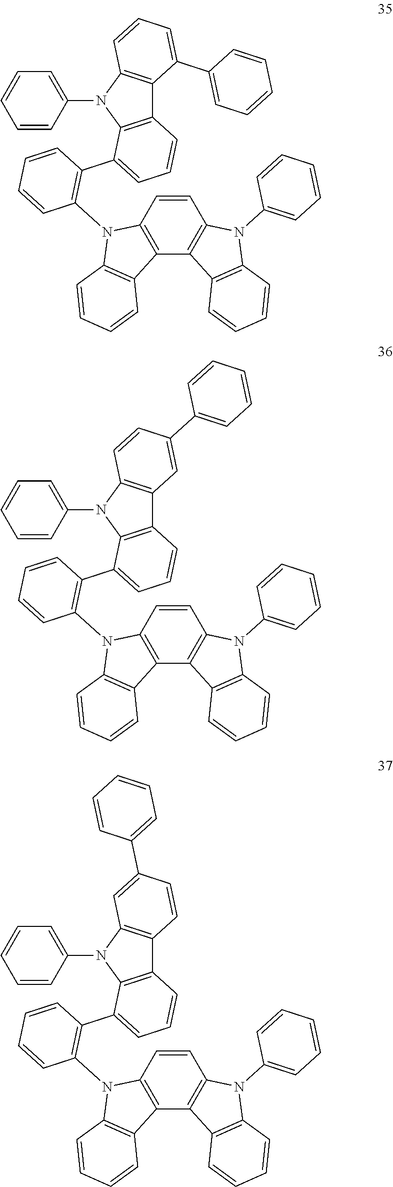 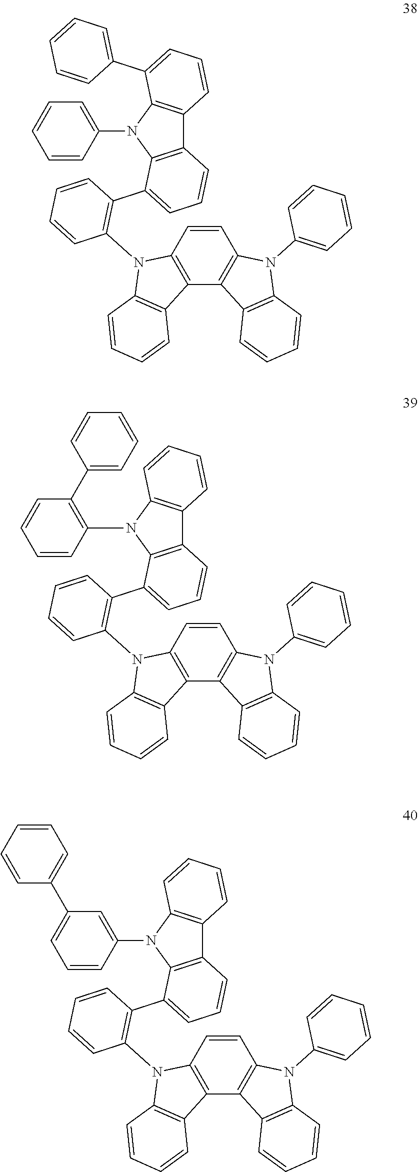 [SMILES: c1ccc(-c2cccc3c4cccc(-c5ccccc5-n5c6ccccc6c6c7c8ccccc8n(-c8ccccc8)c7ccc65)c4n(-c4ccccc4)c23)cc1 ; c1ccc(-c2ccccc2-n2c3ccccc3c3cccc(-c4ccccc4-n4c5ccccc5c5c6c7ccccc7n(-c7ccccc7)c6ccc54)c32)cc1 ; c1ccc(-c2cccc(-n3c4ccccc4c4cccc(-c5ccccc5-n5c6ccccc6c6c7c8ccccc8n(-c8ccccc8)c7ccc65)c43)c2)cc1] 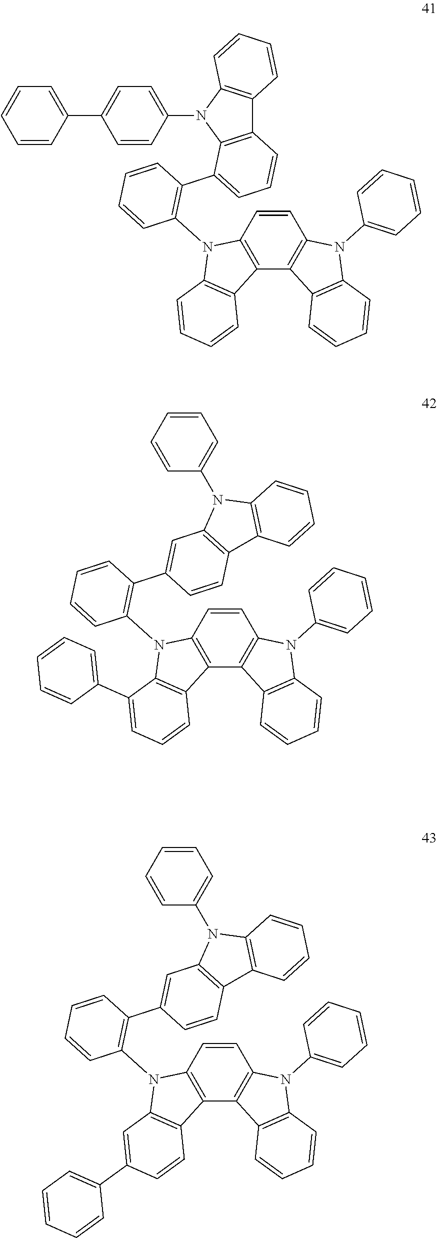 [SMILES: c1ccc(-c2ccc(-n3c4ccccc4c4cccc(-c5ccccc5-n5c6ccccc6c6c7c8ccccc8n(-c8ccccc8)c7ccc65)c43)cc2)cc1 ; c1ccc(-c2cccc3c4c5c6ccccc6n(-c6ccccc6)c5ccc4n(-c4ccccc4-c4ccc5c6ccccc6n(-c6ccccc6)c5c4)c23)cc1 ; c1ccc(-c2ccc3c4c5c6ccccc6n(-c6ccccc6)c5ccc4n(-c4ccccc4-c4ccc5c6ccccc6n(-c6ccccc6)c5c4)c3c2)cc1] 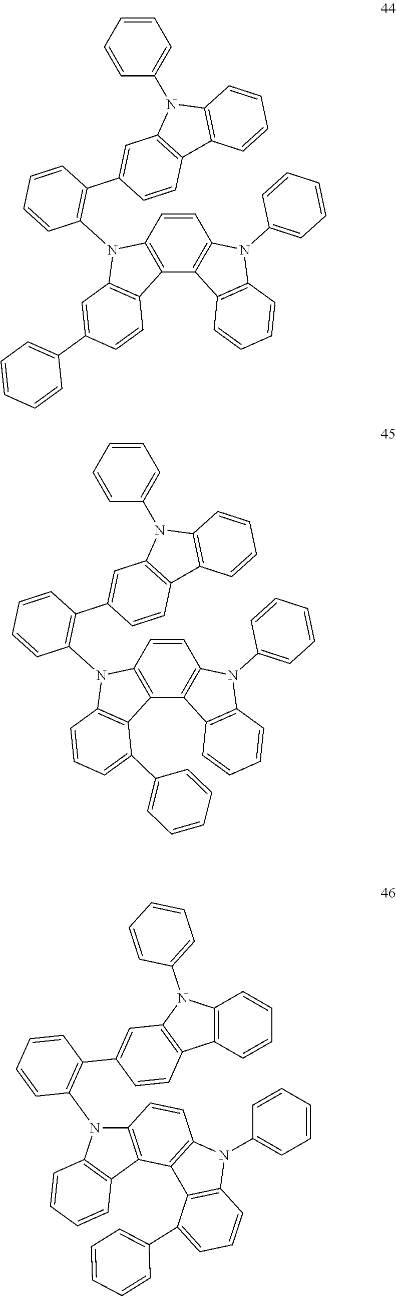 [SMILES: c1ccc(-c2ccc3c4c5c6ccccc6n(-c6ccccc6)c5ccc4n(-c4ccccc4-c4ccc5c6ccccc6n(-c6ccccc6)c5c4)c3c2)cc1 ; c1ccc(-c2cccc3c2c2c4c5ccccc5n(-c5ccccc5)c4ccc2n3-c2ccccc2-c2ccc3c4ccccc4n(-c4ccccc4)c3c2)cc1 ; c1ccc(-c2cccc3c2c2c4c5ccccc5n(-c5ccccc5-c5ccc6c7ccccc7n(-c7ccccc7)c6c5)c4ccc2n3-c2ccccc2)cc1] 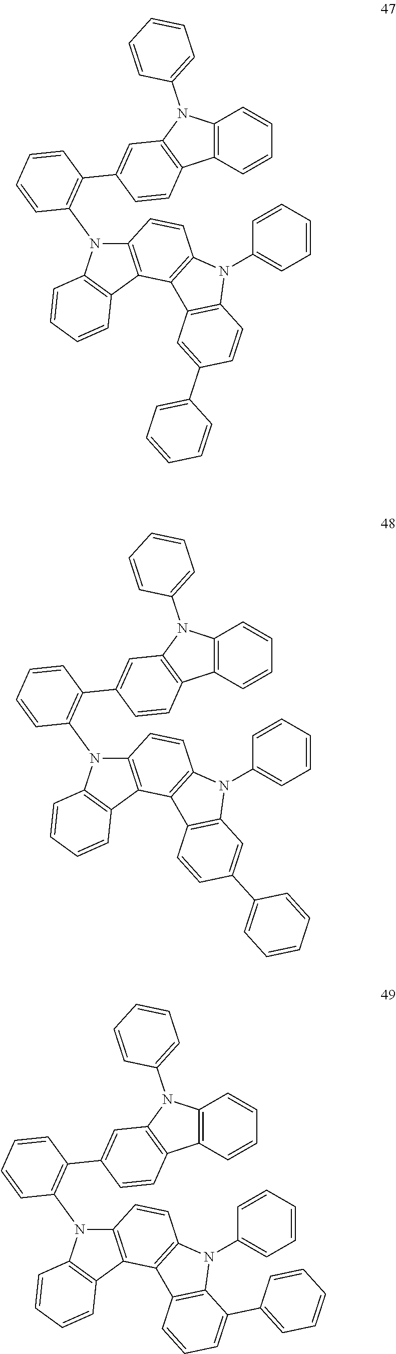 [SMILES: c1ccc(-c2ccc3c(c2)c2c4c5ccccc5n(-c5ccccc5-c5ccc6c7ccccc7n(-c7ccccc7)c6c5)c4ccc2n3-c2ccccc2)cc1 ; c1ccc(-c2ccc3c4c5c6ccccc6n(-c6ccccc6-c6ccc7c8ccccc8n(-c8ccccc8)c7c6)c5ccc4n(-c4ccccc4)c3c2)cc1 ; c1ccc(-c2cccc3c4c5c6ccccc6n(-c6ccccc6-c6ccc7c8ccccc8n(-c8ccccc8)c7c6)c5ccc4n(-c4ccccc4)c23)cc1] 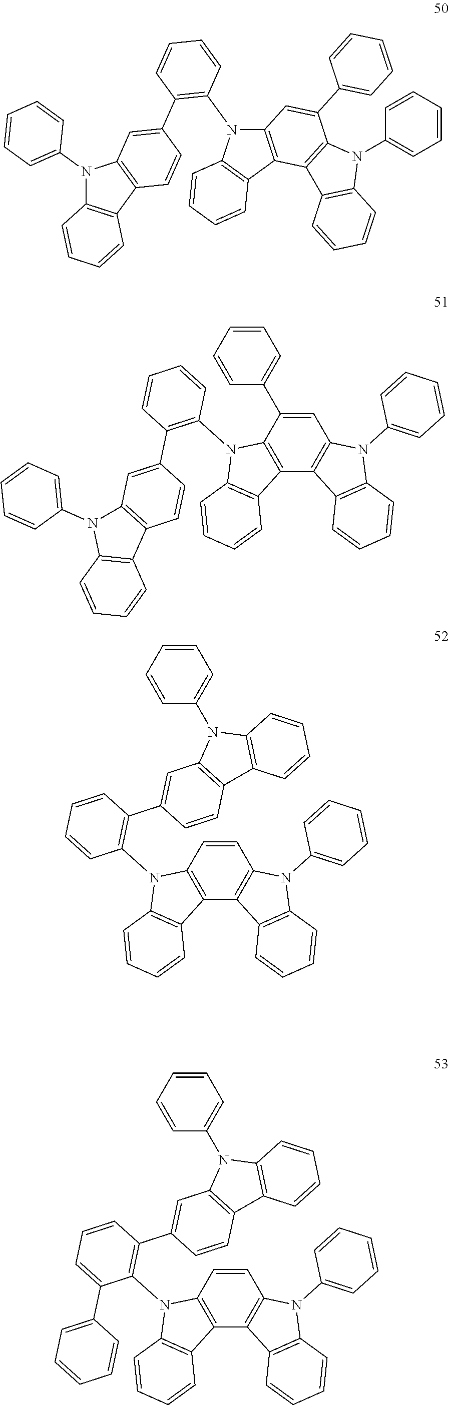 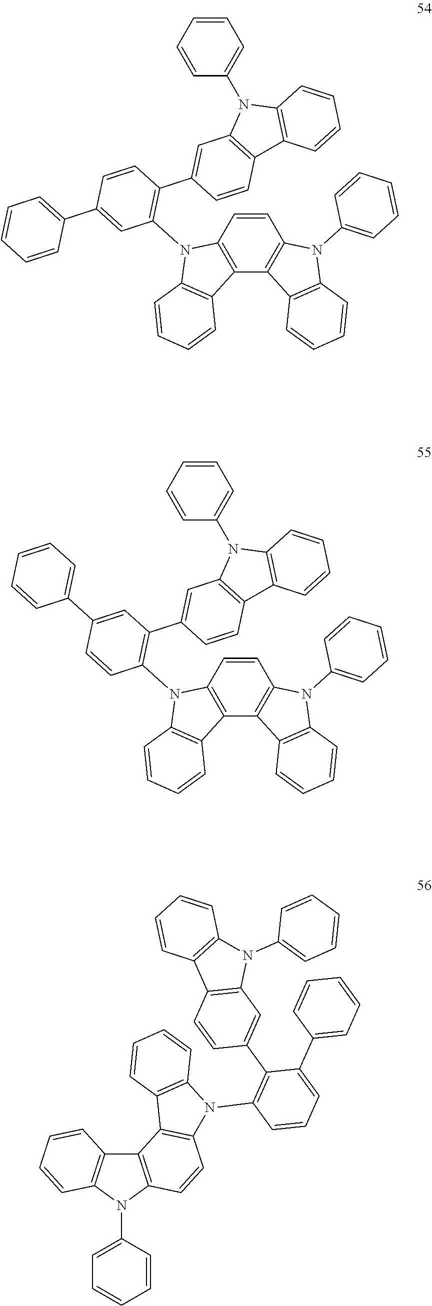 [SMILES: c1ccc(-c2ccc(-c3ccc4c5ccccc5n(-c5ccccc5)c4c3)c(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccccc6)c5ccc43)c(-c3ccc4c5ccccc5n(-c5ccccc5)c4c3)c2)cc1 ; c1ccc(-c2cccc(-n3c4ccccc4c4c5c6ccccc6n(-c6ccccc6)c5ccc43)c2-c2ccc3c4ccccc4n(-c4ccccc4)c3c2)cc1] 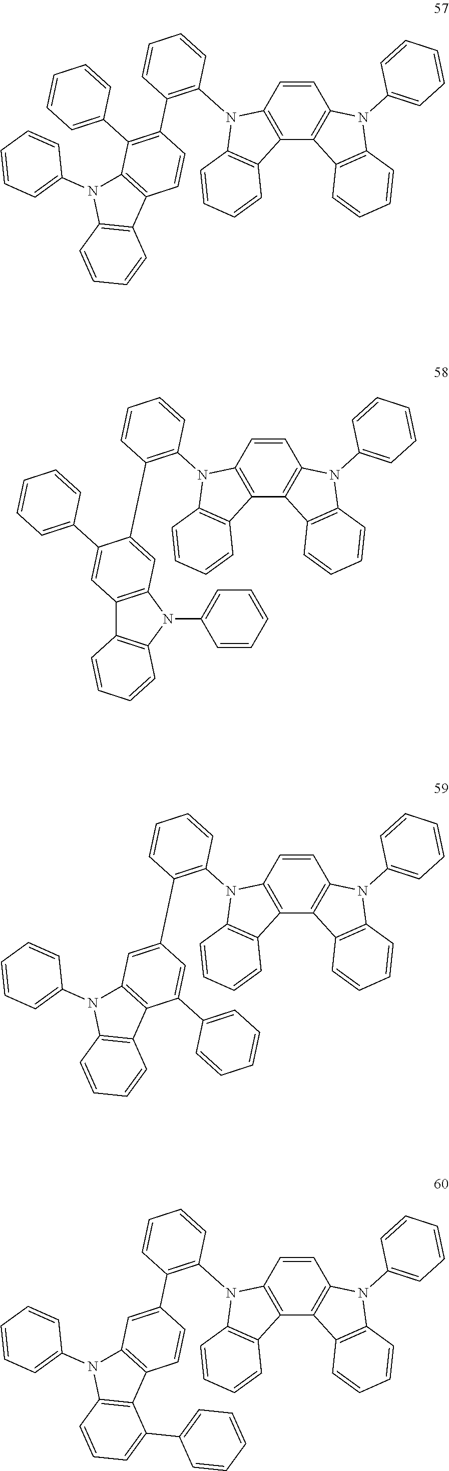 [SMILES: c1ccc(-c2c(-c3ccccc3-n3c4ccccc4c4c5c6ccccc6n(-c6ccccc6)c5ccc43)ccc3c4ccccc4n(-c4ccccc4)c23)cc1 ; c1ccc(-c2cc3c4ccccc4n(-c4ccccc4)c3cc2-c2ccccc2-n2c3ccccc3c3c4c5ccccc5n(-c5ccccc5)c4ccc32)cc1 ; c1ccc(-c2cc(-c3ccccc3-n3c4ccccc4c4c5c6ccccc6n(-c6ccccc6)c5ccc43)cc3c2c2ccccc2n3-c2ccccc2)cc1 ; c1ccc(-c2cccc3c2c2ccc(-c4ccccc4-n4c5ccccc5c5c6c7ccccc7n(-c7ccccc7)c6ccc54)cc2n3-c2ccccc2)cc1] 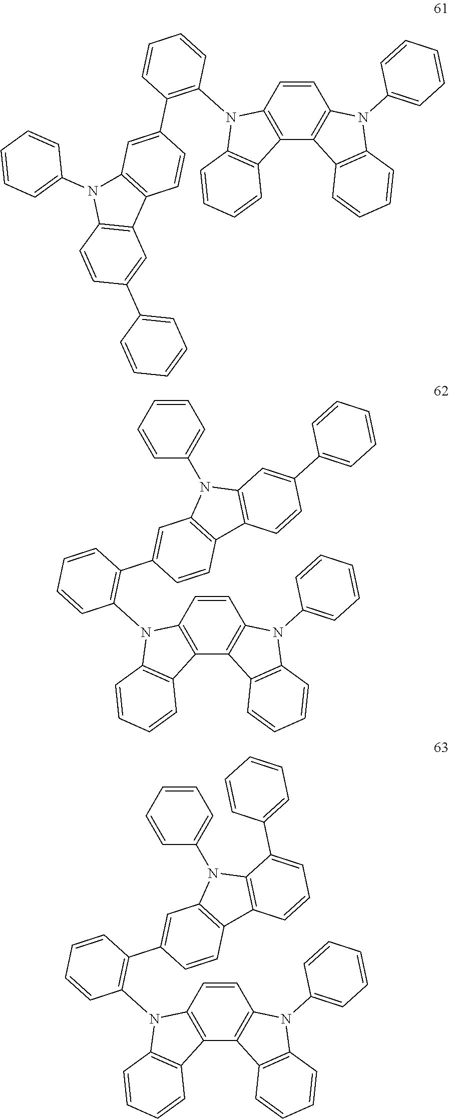 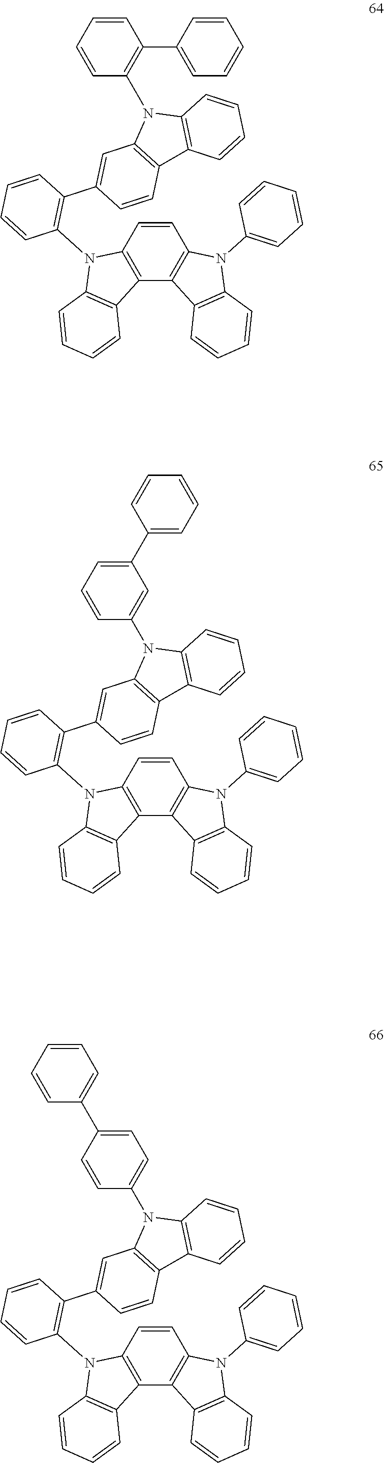 [SMILES: c1ccc(-c2ccccc2-n2c3ccccc3c3ccc(-c4ccccc4-n4c5ccccc5c5c6c7ccccc7n(-c7ccccc7)c6ccc54)cc32)cc1 ; c1ccc(-c2cccc(-n3c4ccccc4c4ccc(-c5ccccc5-n5c6ccccc6c6c7c8ccccc8n(-c8ccccc8)c7ccc65)cc43)c2)cc1 ; c1ccc(-c2ccc(-n3c4ccccc4c4ccc(-c5ccccc5-n5c6ccccc6c6c7c8ccccc8n(-c8ccccc8)c7ccc65)cc43)cc2)cc1] 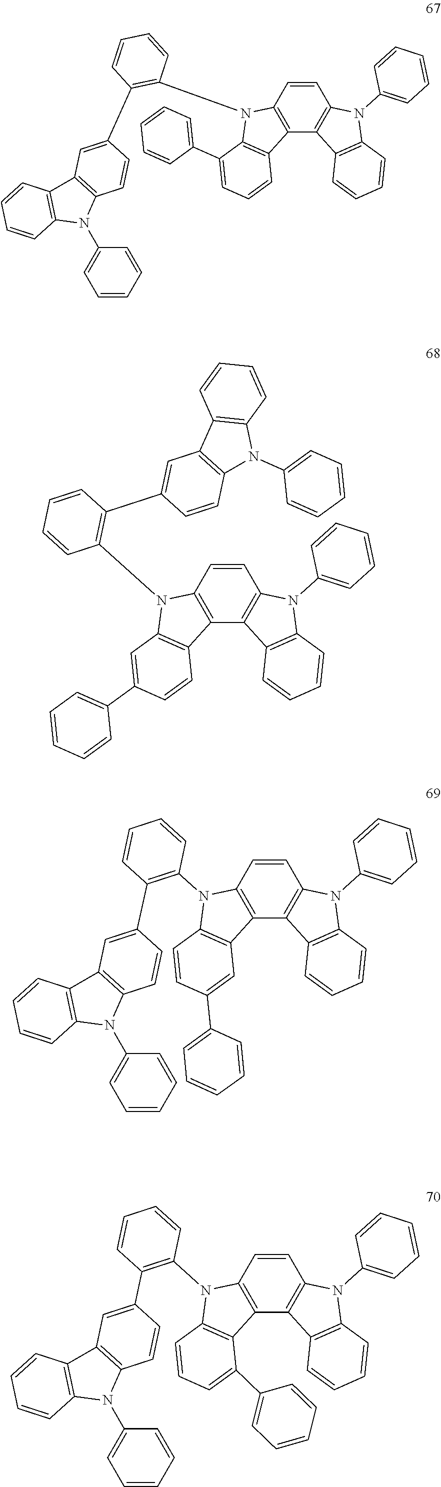 [SMILES: c1ccc(-c2cccc3c4c5c6ccccc6n(-c6ccccc6)c5ccc4n(-c4ccccc4-c4ccc5c(c4)c4ccccc4n5-c4ccccc4)c23)cc1 ; c1ccc(-c2ccc3c4c5c6ccccc6n(-c6ccccc6)c5ccc4n(-c4ccccc4-c4ccc5c(c4)c4ccccc4n5-c4ccccc4)c3c2)cc1 ; c1ccc(-c2ccc3c(c2)c2c4c5ccccc5n(-c5ccccc5)c4ccc2n3-c2ccccc2-c2ccc3c(c2)c2ccccc2n3-c2ccccc2)cc1 ; c1ccc(-c2cccc3c2c2c4c5ccccc5n(-c5ccccc5)c4ccc2n3-c2ccccc2-c2ccc3c(c2)c2ccccc2n3-c2ccccc2)cc1] 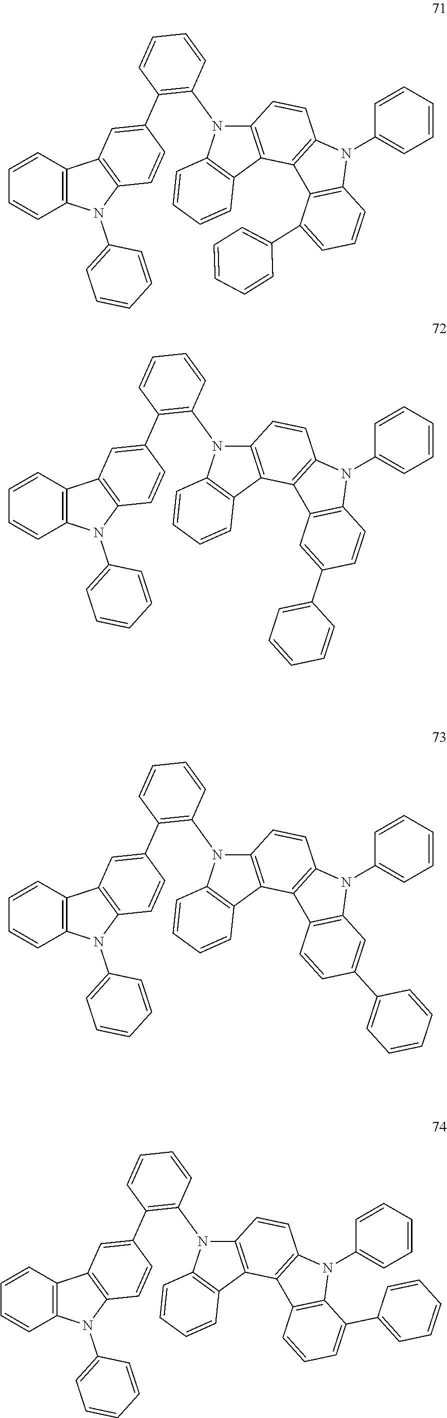 [SMILES: c1ccc(-c2cccc3c2c2c4c5ccccc5n(-c5ccccc5-c5ccc6c(c5)c5ccccc5n6-c5ccccc5)c4ccc2n3-c2ccccc2)cc1 ; c1ccc(-c2ccc3c(c2)c2c4c5ccccc5n(-c5ccccc5-c5ccc6c(c5)c5ccccc5n6-c5ccccc5)c4ccc2n3-c2ccccc2)cc1 ; c1ccc(-c2ccc3c4c5c6ccccc6n(-c6ccccc6-c6ccc7c(c6)c6ccccc6n7-c6ccccc6)c5ccc4n(-c4ccccc4)c3c2)cc1 ; c1ccc(-c2cccc3c4c5c6ccccc6n(-c6ccccc6-c6ccc7c(c6)c6ccccc6n7-c6ccccc6)c5ccc4n(-c4ccccc4)c23)cc1] 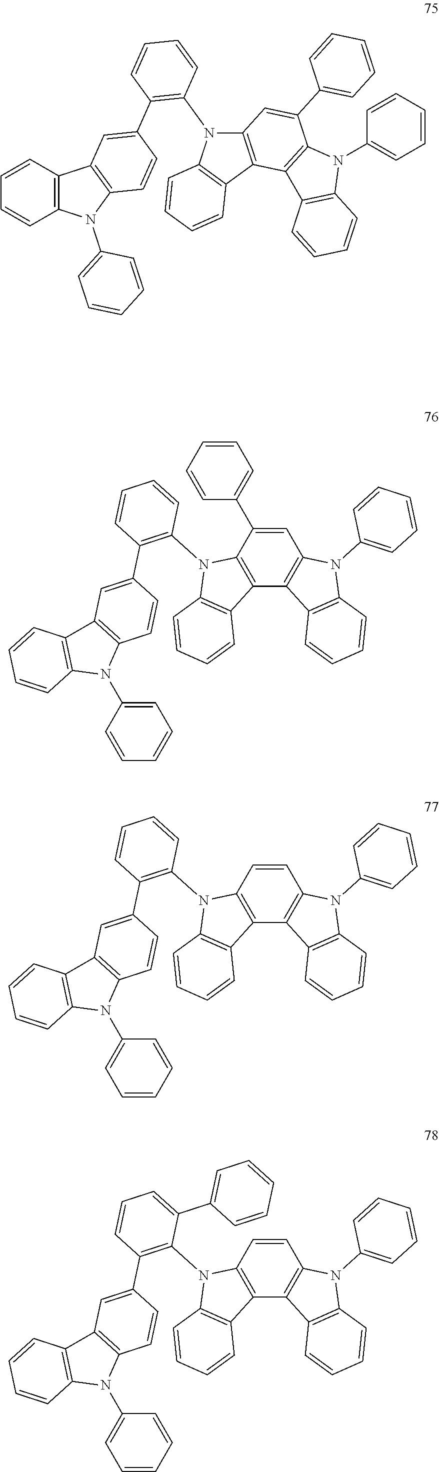 [SMILES: c1ccc(-c2cc3c(c4ccccc4n3-c3ccccc3-c3ccc4c(c3)c3ccccc3n4-c3ccccc3)c3c4ccccc4n(-c4ccccc4)c23)cc1 ; c1ccc(-c2cc3c(c4ccccc4n3-c3ccccc3)c3c4ccccc4n(-c4ccccc4-c4ccc5c(c4)c4ccccc4n5-c4ccccc4)c23)cc1 ; c1ccc(-n2c3ccccc3c3cc(-c4ccccc4-n4c5ccccc5c5c6c7ccccc7n(-c7ccccc7)c6ccc54)ccc32)cc1 ; c1ccc(-c2cccc(-c3ccc4c(c3)c3ccccc3n4-c3ccccc3)c2-n2c3ccccc3c3c4c5ccccc5n(-c5ccccc5)c4ccc32)cc1] 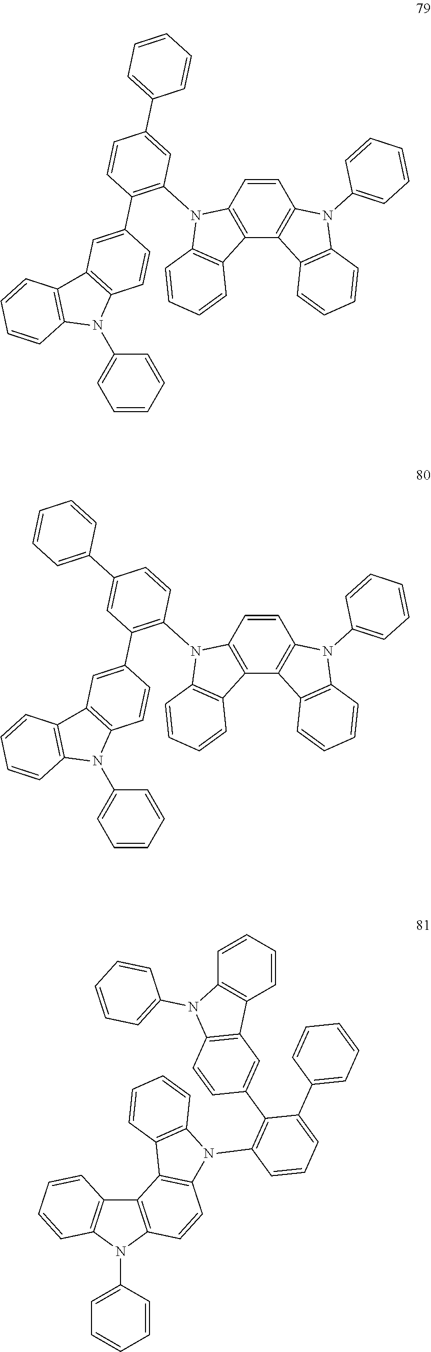 [SMILES: c1ccc(-c2ccc(-c3ccc4c(c3)c3ccccc3n4-c3ccccc3)c(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccccc6)c5ccc43)c(-c3ccc4c(c3)c3ccccc3n4-c3ccccc3)c2)cc1 ; c1ccc(-c2cccc(-n3c4ccccc4c4c5c6ccccc6n(-c6ccccc6)c5ccc43)c2-c2ccc3c(c2)c2ccccc2n3-c2ccccc2)cc1] 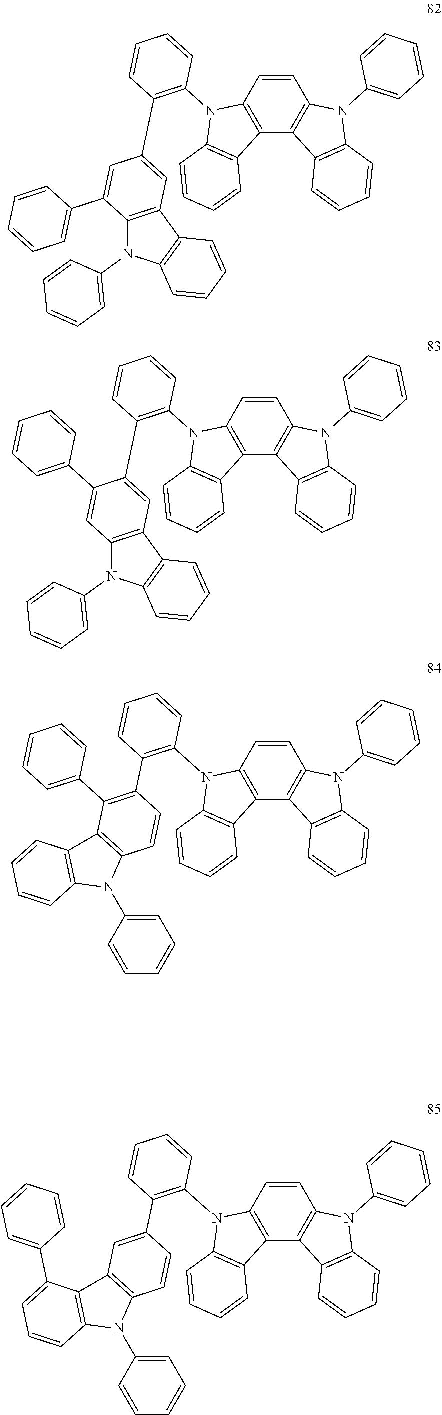 [SMILES: c1ccc(-c2cc(-c3ccccc3-n3c4ccccc4c4c5c6ccccc6n(-c6ccccc6)c5ccc43)cc3c4ccccc4n(-c4ccccc4)c23)cc1 ; c1ccc(-c2cc3c(cc2-c2ccccc2-n2c4ccccc4c4c5c6ccccc6n(-c6ccccc6)c5ccc42)c2ccccc2n3-c2ccccc2)cc1 ; c1ccc(-c2c(-c3ccccc3-n3c4ccccc4c4c5c6ccccc6n(-c6ccccc6)c5ccc43)ccc3c2c2ccccc2n3-c2ccccc2)cc1 ; c1ccc(-c2cccc3c2c2cc(-c4ccccc4-n4c5ccccc5c5c6c7ccccc7n(-c7ccccc7)c6ccc54)ccc2n3-c2ccccc2)cc1] 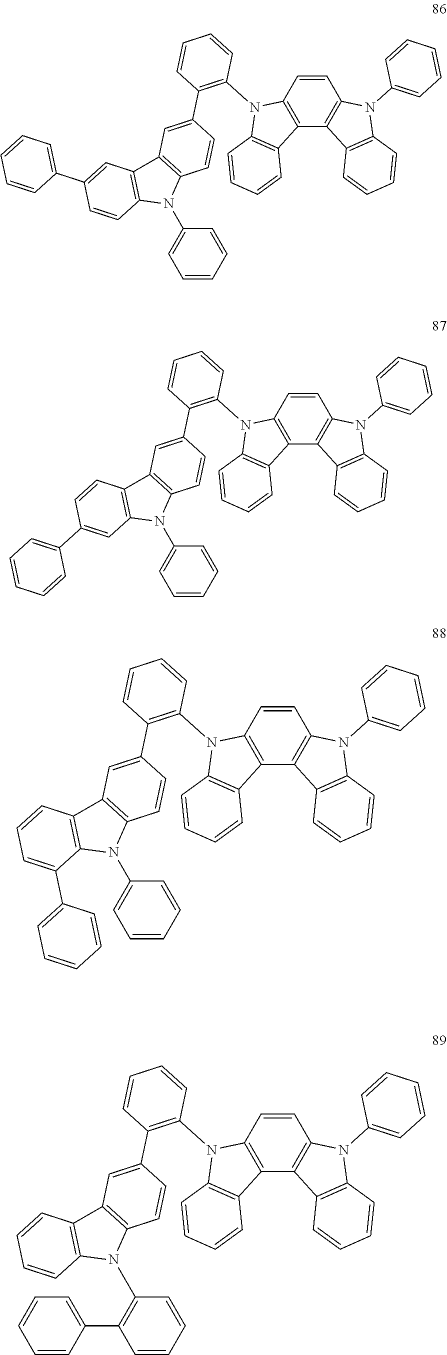 [SMILES: c1ccc(-c2ccc3c(c2)c2cc(-c4ccccc4-n4c5ccccc5c5c6c7ccccc7n(-c7ccccc7)c6ccc54)ccc2n3-c2ccccc2)cc1 ; c1ccc(-c2ccc3c4cc(-c5ccccc5-n5c6ccccc6c6c7c8ccccc8n(-c8ccccc8)c7ccc65)ccc4n(-c4ccccc4)c3c2)cc1 ; c1ccc(-c2cccc3c4cc(-c5ccccc5-n5c6ccccc6c6c7c8ccccc8n(-c8ccccc8)c7ccc65)ccc4n(-c4ccccc4)c23)cc1 ; c1ccc(-c2ccccc2-n2c3ccccc3c3cc(-c4ccccc4-n4c5ccccc5c5c6c7ccccc7n(-c7ccccc7)c6ccc54)ccc32)cc1] 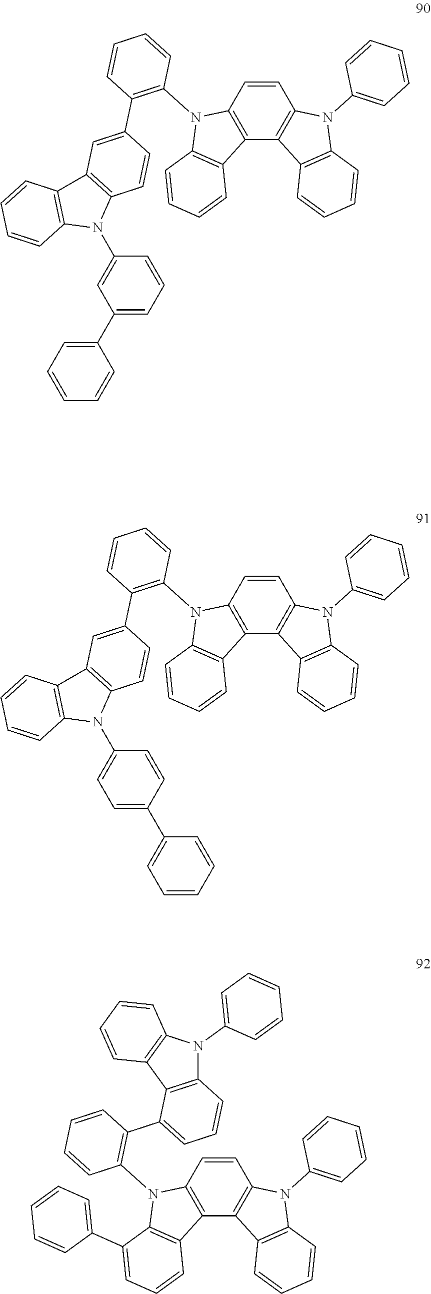 [SMILES: c1ccc(-c2cccc(-n3c4ccccc4c4cc(-c5ccccc5-n5c6ccccc6c6c7c8ccccc8n(-c8ccccc8)c7ccc65)ccc43)c2)cc1 ; c1ccc(-c2ccc(-n3c4ccccc4c4cc(-c5ccccc5-n5c6ccccc6c6c7c8ccccc8n(-c8ccccc8)c7ccc65)ccc43)cc2)cc1 ; c1ccc(-c2cccc3c4c5c6ccccc6n(-c6ccccc6)c5ccc4n(-c4ccccc4-c4cccc5c4c4ccccc4n5-c4ccccc4)c23)cc1] 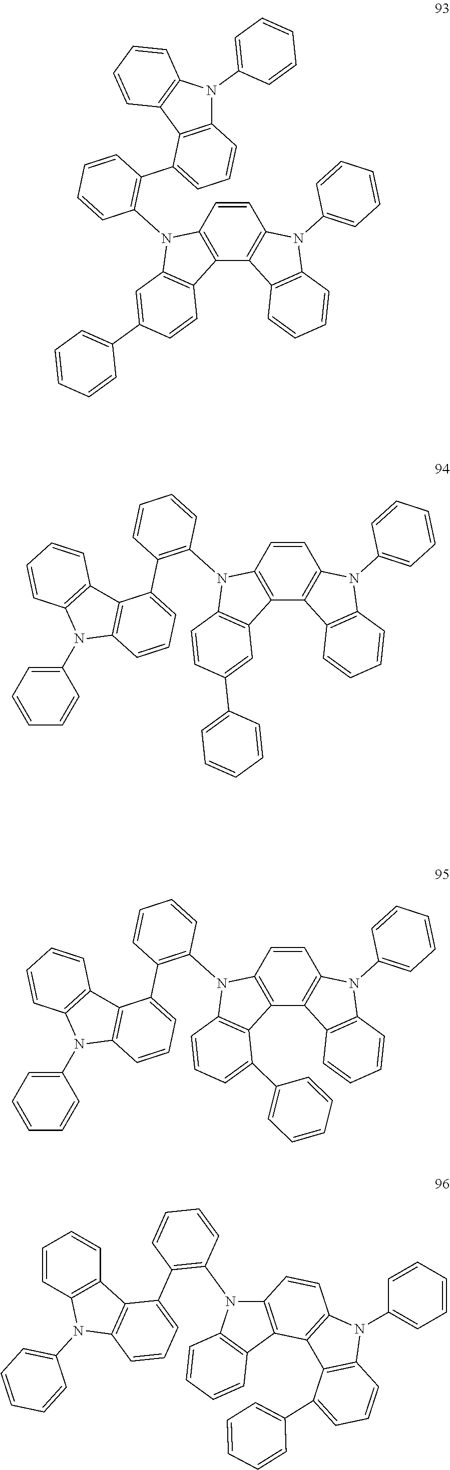 [SMILES: c1ccc(-c2ccc3c4c5c6ccccc6n(-c6ccccc6)c5ccc4n(-c4ccccc4-c4cccc5c4c4ccccc4n5-c4ccccc4)c3c2)cc1 ; c1ccc(-c2ccc3c(c2)c2c4c5ccccc5n(-c5ccccc5)c4ccc2n3-c2ccccc2-c2cccc3c2c2ccccc2n3-c2ccccc2)cc1 ; c1ccc(-c2cccc3c2c2c4c5ccccc5n(-c5ccccc5)c4ccc2n3-c2ccccc2-c2cccc3c2c2ccccc2n3-c2ccccc2)cc1 ; c1ccc(-c2cccc3c2c2c4c5ccccc5n(-c5ccccc5-c5cccc6c5c5ccccc5n6-c5ccccc5)c4ccc2n3-c2ccccc2)cc1] 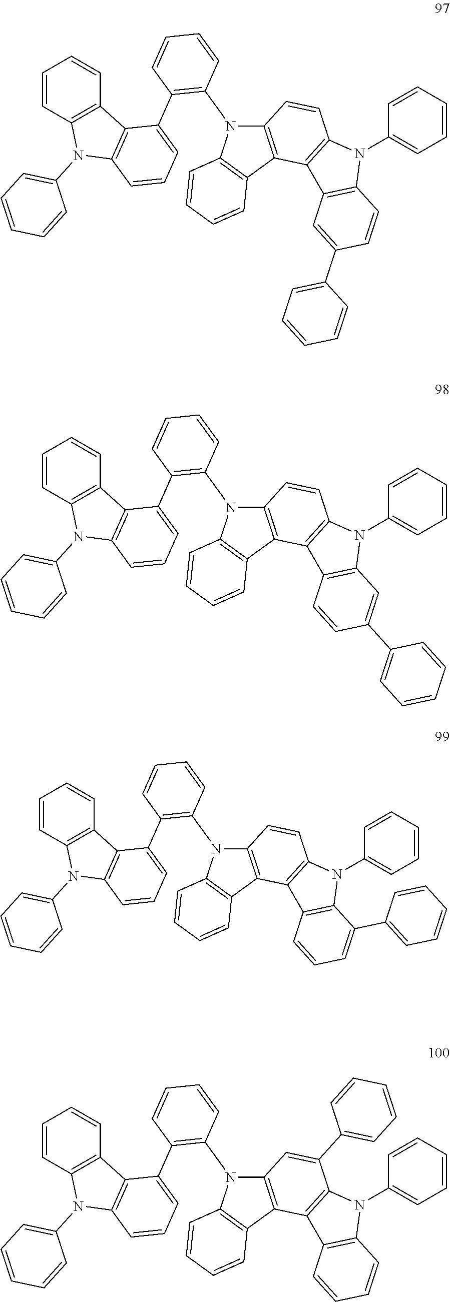 [SMILES: c1ccc(-c2ccc3c(c2)c2c4c5ccccc5n(-c5ccccc5-c5cccc6c5c5ccccc5n6-c5ccccc5)c4ccc2n3-c2ccccc2)cc1 ; c1ccc(-c2ccc3c4c5c6ccccc6n(-c6ccccc6-c6cccc7c6c6ccccc6n7-c6ccccc6)c5ccc4n(-c4ccccc4)c3c2)cc1 ; c1ccc(-c2cccc3c4c5c6ccccc6n(-c6ccccc6-c6cccc7c6c6ccccc6n7-c6ccccc6)c5ccc4n(-c4ccccc4)c23)cc1 ; c1ccc(-c2cc3c(c4ccccc4n3-c3ccccc3-c3cccc4c3c3ccccc3n4-c3ccccc3)c3c4ccccc4n(-c4ccccc4)c23)cc1] 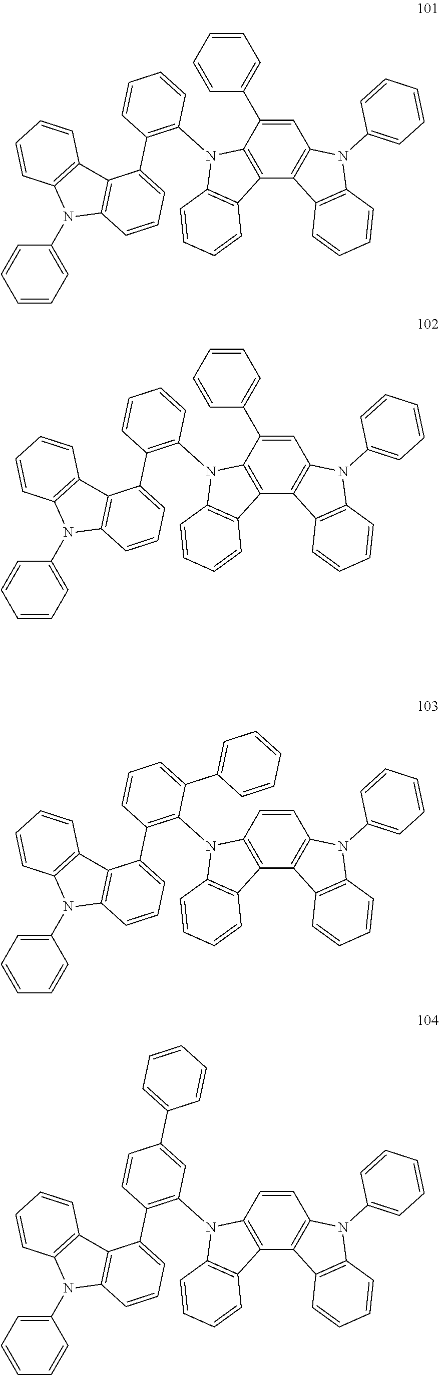 [SMILES: c1ccc(-c2cc3c(c4ccccc4n3-c3ccccc3)c3c4ccccc4n(-c4ccccc4-c4cccc5c4c4ccccc4n5-c4ccccc4)c23)cc1 ; c1ccc(-c2cc3c(c4ccccc4n3-c3ccccc3)c3c4ccccc4n(-c4ccccc4-c4cccc5c4c4ccccc4n5-c4ccccc4)c23)cc1 ; c1ccc(-c2cccc(-c3cccc4c3c3ccccc3n4-c3ccccc3)c2-n2c3ccccc3c3c4c5ccccc5n(-c5ccccc5)c4ccc32)cc1 ; c1ccc(-c2ccc(-c3cccc4c3c3ccccc3n4-c3ccccc3)c(-n3c4ccccc4c4c5c6ccccc6n(-c6ccccc6)c5ccc43)c2)cc1] 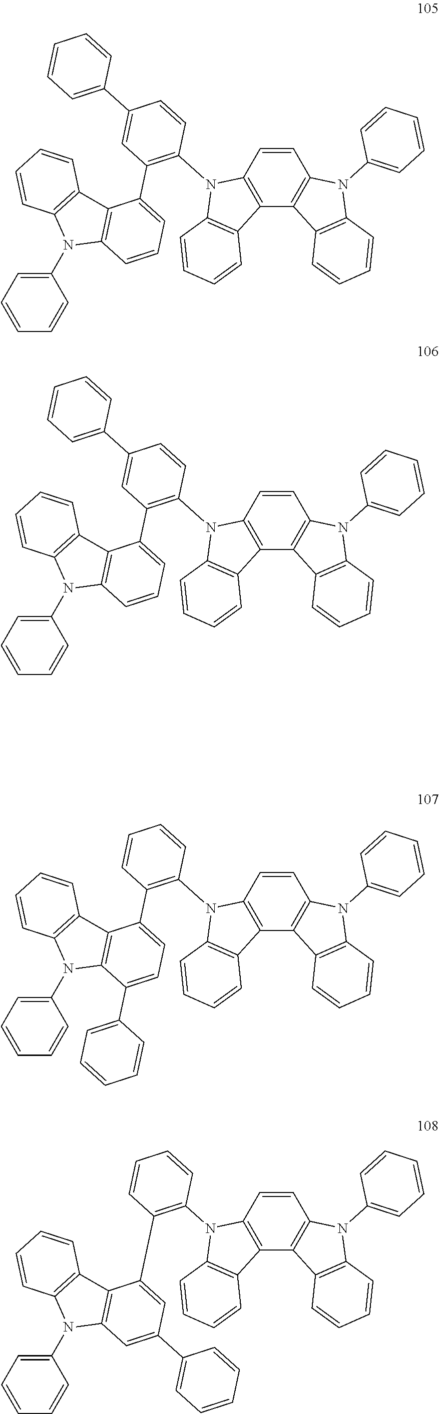 [SMILES: c1ccc(-c2ccc(-n3c4ccccc4c4c5c6ccccc6n(-c6ccccc6)c5ccc43)c(-c3cccc4c3c3ccccc3n4-c3ccccc3)c2)cc1 ; c1ccc(-c2ccc(-n3c4ccccc4c4c5c6ccccc6n(-c6ccccc6)c5ccc43)c(-c3cccc4c3c3ccccc3n4-c3ccccc3)c2)cc1 ; c1ccc(-c2ccc(-c3ccccc3-n3c4ccccc4c4c5c6ccccc6n(-c6ccccc6)c5ccc43)c3c4ccccc4n(-c4ccccc4)c23)cc1 ; c1ccc(-c2cc(-c3ccccc3-n3c4ccccc4c4c5c6ccccc6n(-c6ccccc6)c5ccc43)c3c4ccccc4n(-c4ccccc4)c3c2)cc1] 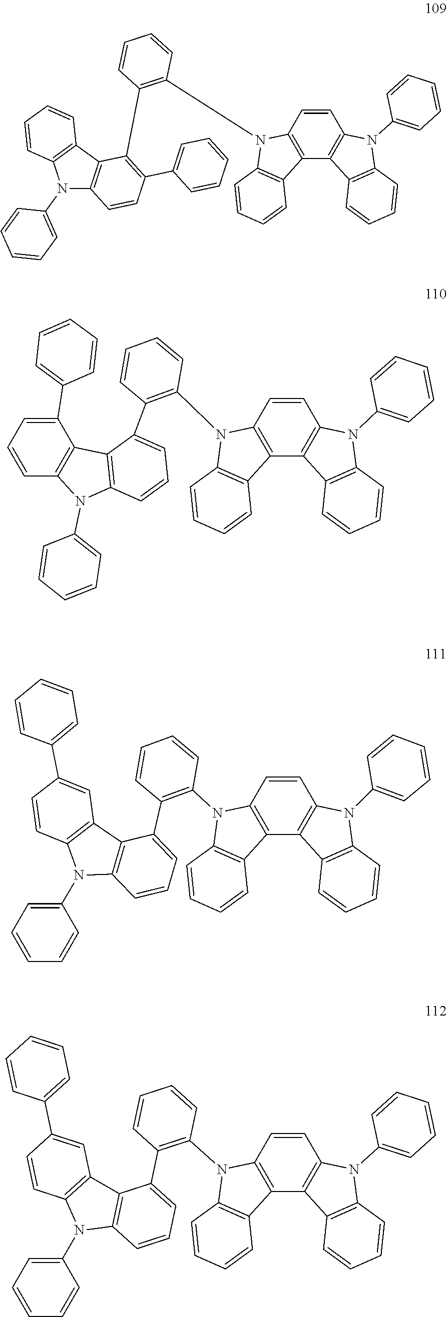 [SMILES: c1ccc(-c2ccc3c(c2-c2ccccc2-n2c4ccccc4c4c5c6ccccc6n(-c6ccccc6)c5ccc42)c2ccccc2n3-c2ccccc2)cc1 ; c1ccc(-c2cccc3c2c2c(-c4ccccc4-n4c5ccccc5c5c6c7ccccc7n(-c7ccccc7)c6ccc54)cccc2n3-c2ccccc2)cc1 ; c1ccc(-c2ccc3c(c2)c2c(-c4ccccc4-n4c5ccccc5c5c6c7ccccc7n(-c7ccccc7)c6ccc54)cccc2n3-c2ccccc2)cc1 ; c1ccc(-c2ccc3c(c2)c2c(-c4ccccc4-n4c5ccccc5c5c6c7ccccc7n(-c7ccccc7)c6ccc54)cccc2n3-c2ccccc2)cc1] 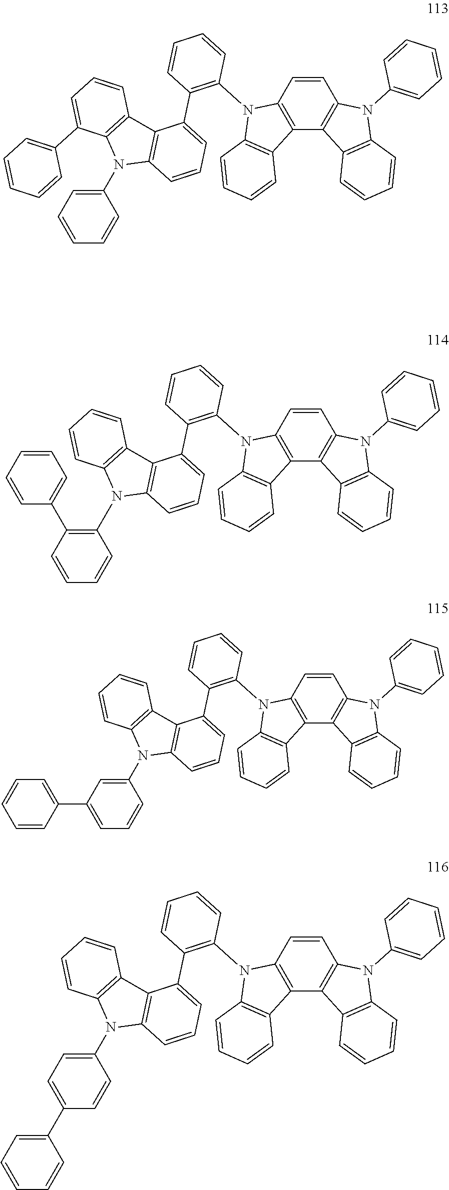 [SMILES: c1ccc(-c2cccc3c4c(-c5ccccc5-n5c6ccccc6c6c7c8ccccc8n(-c8ccccc8)c7ccc65)cccc4n(-c4ccccc4)c23)cc1 ; c1ccc(-c2ccccc2-n2c3ccccc3c3c(-c4ccccc4-n4c5ccccc5c5c6c7ccccc7n(-c7ccccc7)c6ccc54)cccc32)cc1 ; c1ccc(-c2cccc(-n3c4ccccc4c4c(-c5ccccc5-n5c6ccccc6c6c7c8ccccc8n(-c8ccccc8)c7ccc65)cccc43)c2)cc1 ; c1ccc(-c2ccc(-n3c4ccccc4c4c(-c5ccccc5-n5c6ccccc6c6c7c8ccccc8n(-c8ccccc8)c7ccc65)cccc43)cc2)cc1] 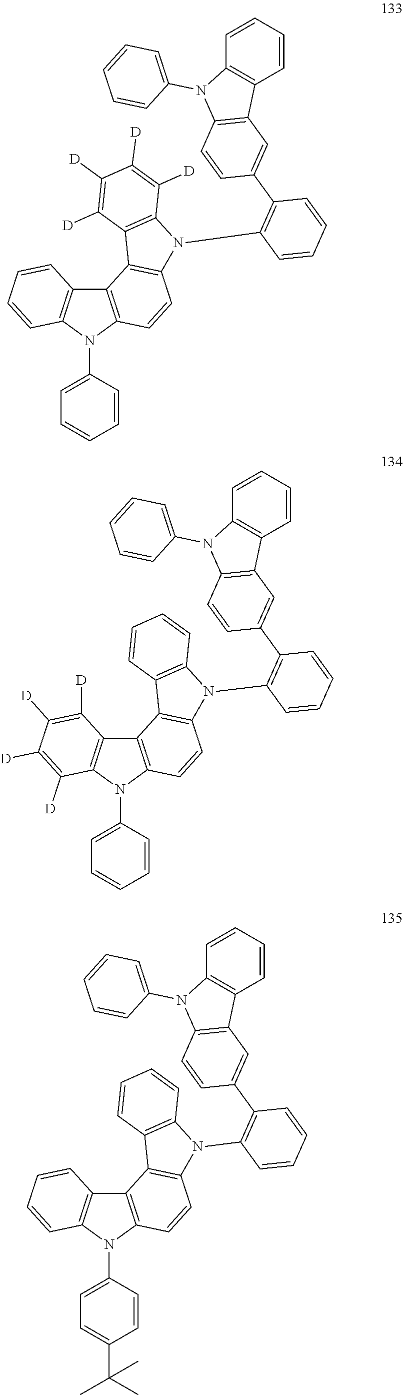 [SMILES: [2H]c1c([2H])c([2H])c2c(c1[2H])c1c3c4ccccc4n(-c4ccccc4)c3ccc1n2-c1ccccc1-c1ccc2c(c1)c1ccccc1n2-c1ccccc1 ; [2H]c1c([2H])c([2H])c2c(c1[2H])c1c3c4ccccc4n(-c4ccccc4-c4ccc5c(c4)c4ccccc4n5-c4ccccc4)c3ccc1n2-c1ccccc1 ; CC(C)(C)c1ccc(-n2c3ccccc3c3c4c5ccccc5n(-c5ccccc5-c5ccc6c(c5)c5ccccc5n6-c5ccccc5)c4ccc32)cc1] 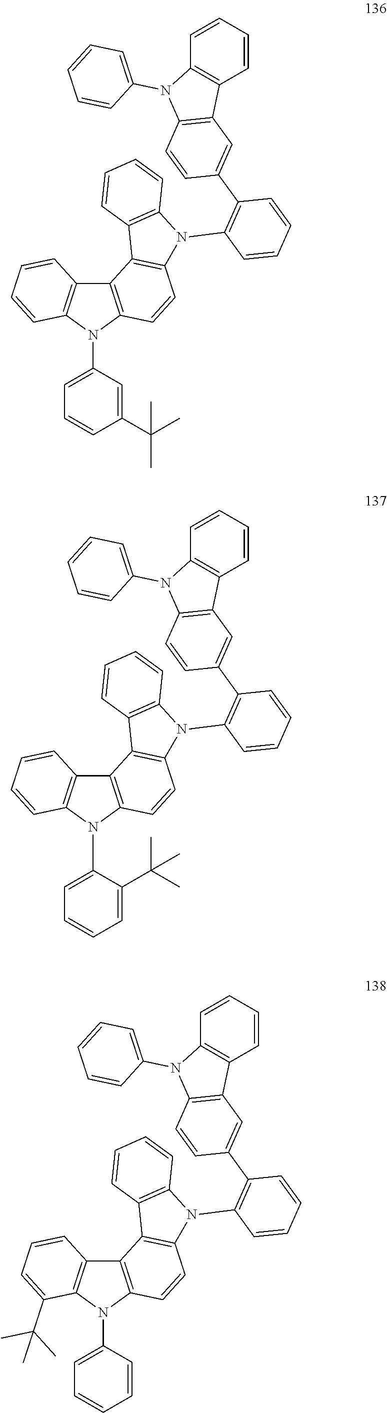 [SMILES: CC(C)(C)c1cccc(-n2c3ccccc3c3c4c5ccccc5n(-c5ccccc5-c5ccc6c(c5)c5ccccc5n6-c5ccccc5)c4ccc32)c1 ; CC(C)(C)c1ccccc1-n1c2ccccc2c2c3c4ccccc4n(-c4ccccc4-c4ccc5c(c4)c4ccccc4n5-c4ccccc4)c3ccc21 ; CC(C)(C)c1cccc2c3c4c5ccccc5n(-c5ccccc5-c5ccc6c(c5)c5ccccc5n6-c5ccccc5)c4ccc3n(-c3ccccc3)c12] 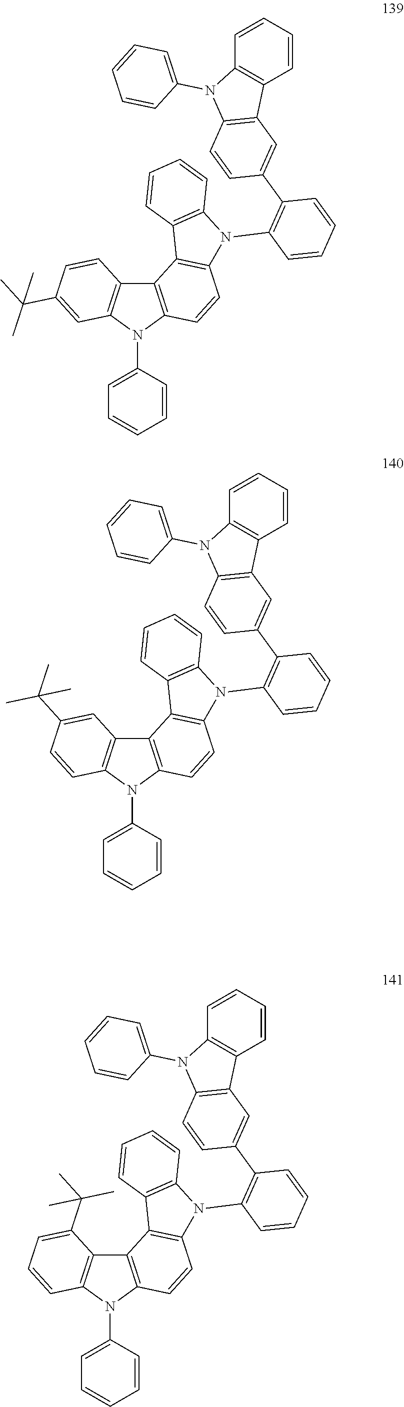 [SMILES: CC(C)(C)c1ccc2c3c4c5ccccc5n(-c5ccccc5-c5ccc6c(c5)c5ccccc5n6-c5ccccc5)c4ccc3n(-c3ccccc3)c2c1 ; CC(C)(C)c1ccc2c(c1)c1c3c4ccccc4n(-c4ccccc4-c4ccc5c(c4)c4ccccc4n5-c4ccccc4)c3ccc1n2-c1ccccc1 ; CC(C)(C)c1cccc2c1c1c3c4ccccc4n(-c4ccccc4-c4ccc5c(c4)c4ccccc4n5-c4ccccc4)c3ccc1n2-c1ccccc1] 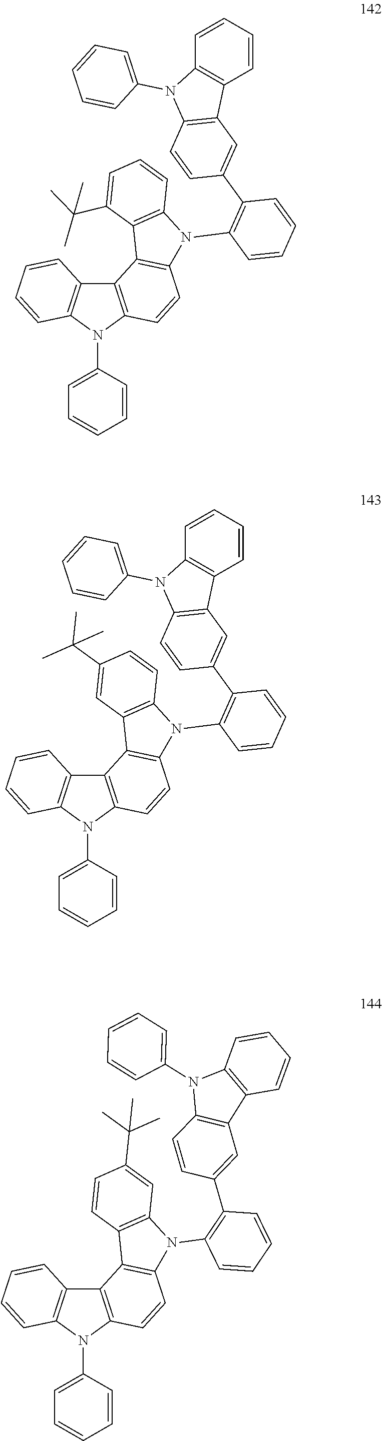 [SMILES: CC(C)(C)c1cccc2c1c1c3c4ccccc4n(-c4ccccc4)c3ccc1n2-c1ccccc1-c1ccc2c(c1)c1ccccc1n2-c1ccccc1 ; CC(C)(C)c1ccc2c(c1)c1c3c4ccccc4n(-c4ccccc4)c3ccc1n2-c1ccccc1-c1ccc2c(c1)c1ccccc1n2-c1ccccc1 ; CC(C)(C)c1ccc2c3c4c5ccccc5n(-c5ccccc5)c4ccc3n(-c3ccccc3-c3ccc4c(c3)c3ccccc3n4-c3ccccc3)c2c1] 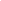 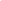 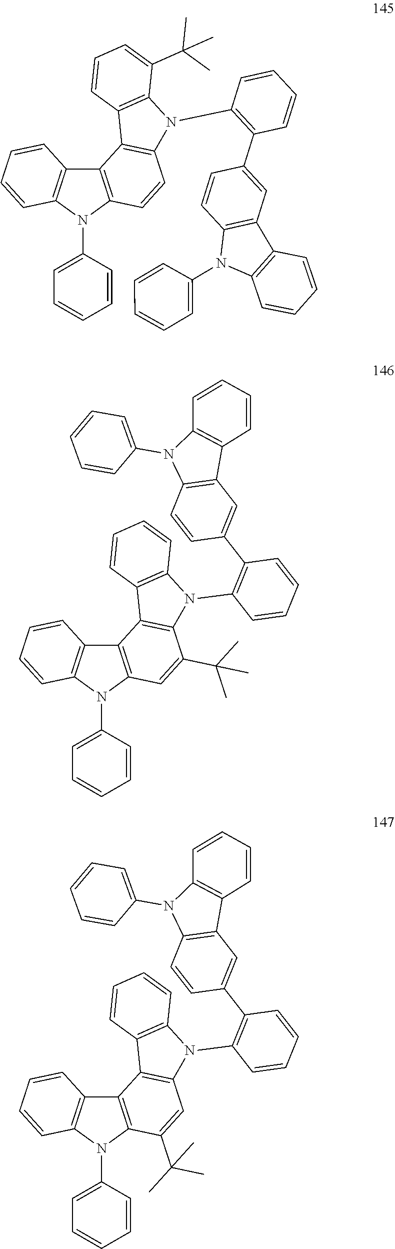 [SMILES: CC(C)(C)c1cccc2c3c4c5ccccc5n(-c5ccccc5)c4ccc3n(-c3ccccc3-c3ccc4c(c3)c3ccccc3n4-c3ccccc3)c12 ; CC(C)(C)c1cc2c(c3ccccc3n2-c2ccccc2)c2c3ccccc3n(-c3ccccc3-c3ccc4c(c3)c3ccccc3n4-c3ccccc3)c12 ; CC(C)(C)c1cc2c(c3ccccc3n2-c2ccccc2-c2ccc3c(c2)c2ccccc2n3-c2ccccc2)c2c3ccccc3n(-c3ccccc3)c12] 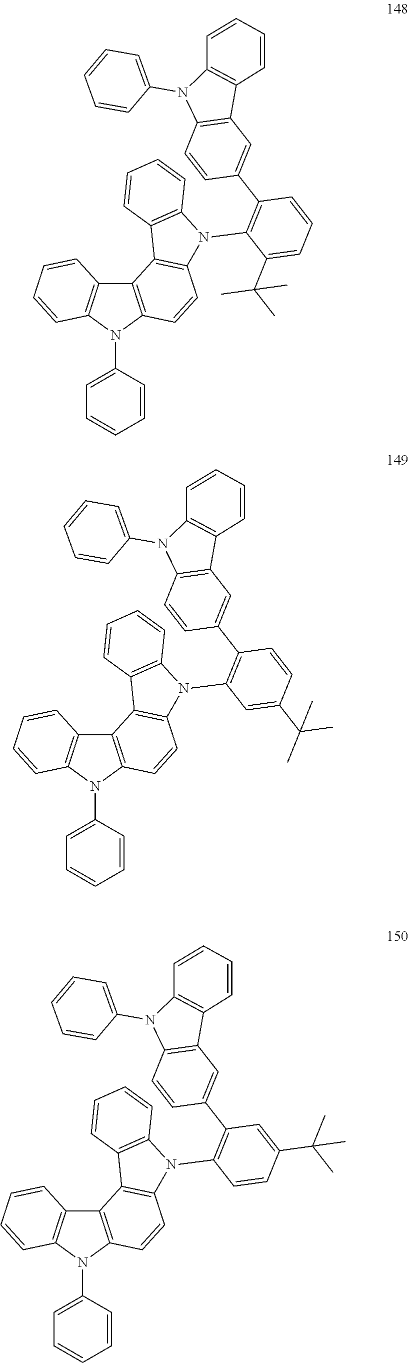 [SMILES: CC(C)(C)c1cccc(-c2ccc3c(c2)c2ccccc2n3-c2ccccc2)c1-n1c2ccccc2c2c3c4ccccc4n(-c4ccccc4)c3ccc21 ; CC(C)(C)c1ccc(-c2ccc3c(c2)c2ccccc2n3-c2ccccc2)c(-n2c3ccccc3c3c4c5ccccc5n(-c5ccccc5)c4ccc32)c1 ; CC(C)(C)c1ccc(-n2c3ccccc3c3c4c5ccccc5n(-c5ccccc5)c4ccc32)c(-c2ccc3c(c2)c2ccccc2n3-c2ccccc2)c1] 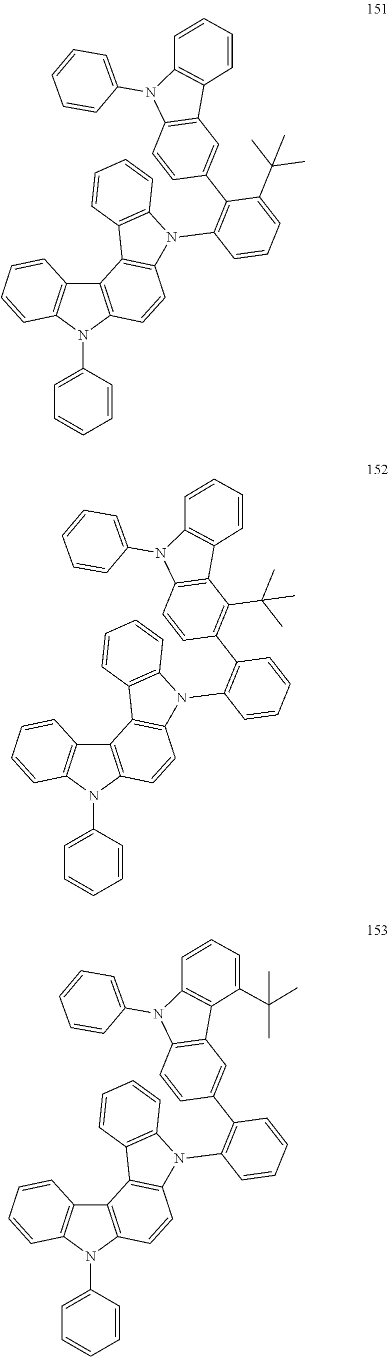 [SMILES: CC(C)(C)c1cccc(-n2c3ccccc3c3c4c5ccccc5n(-c5ccccc5)c4ccc32)c1-c1ccc2c(c1)c1ccccc1n2-c1ccccc1 ; CC(C)(C)c1c(-c2ccccc2-n2c3ccccc3c3c4c5ccccc5n(-c5ccccc5)c4ccc32)ccc2c1c1ccccc1n2-c1ccccc1 ; CC(C)(C)c1cccc2c1c1cc(-c3ccccc3-n3c4ccccc4c4c5c6ccccc6n(-c6ccccc6)c5ccc43)ccc1n2-c1ccccc1] 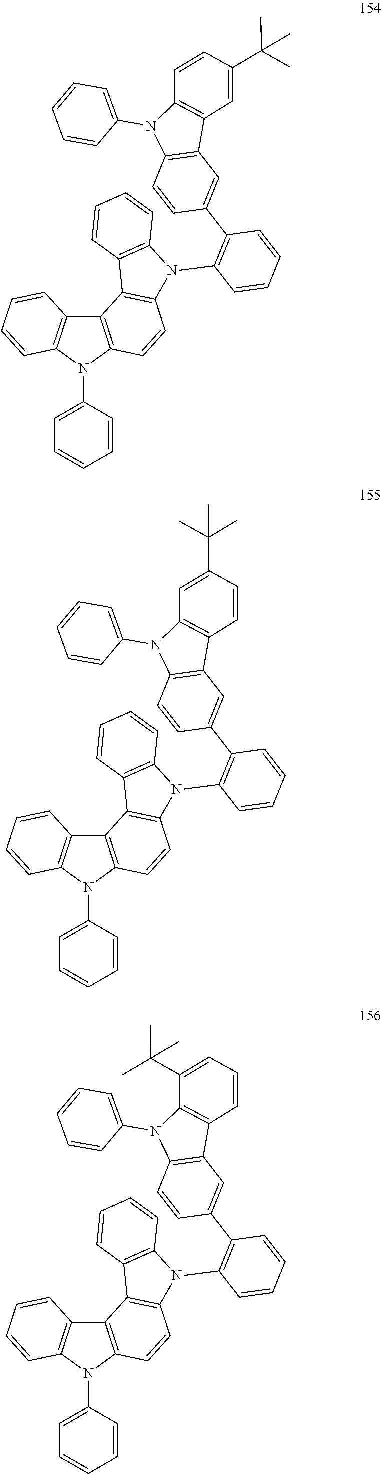 [SMILES: CC(C)(C)c1ccc2c(c1)c1cc(-c3ccccc3-n3c4ccccc4c4c5c6ccccc6n(-c6ccccc6)c5ccc43)ccc1n2-c1ccccc1 ; CC(C)(C)c1ccc2c3cc(-c4ccccc4-n4c5ccccc5c5c6c7ccccc7n(-c7ccccc7)c6ccc54)ccc3n(-c3ccccc3)c2c1 ; CC(C)(C)c1cccc2c3cc(-c4ccccc4-n4c5ccccc5c5c6c7ccccc7n(-c7ccccc7)c6ccc54)ccc3n(-c3ccccc3)c12] 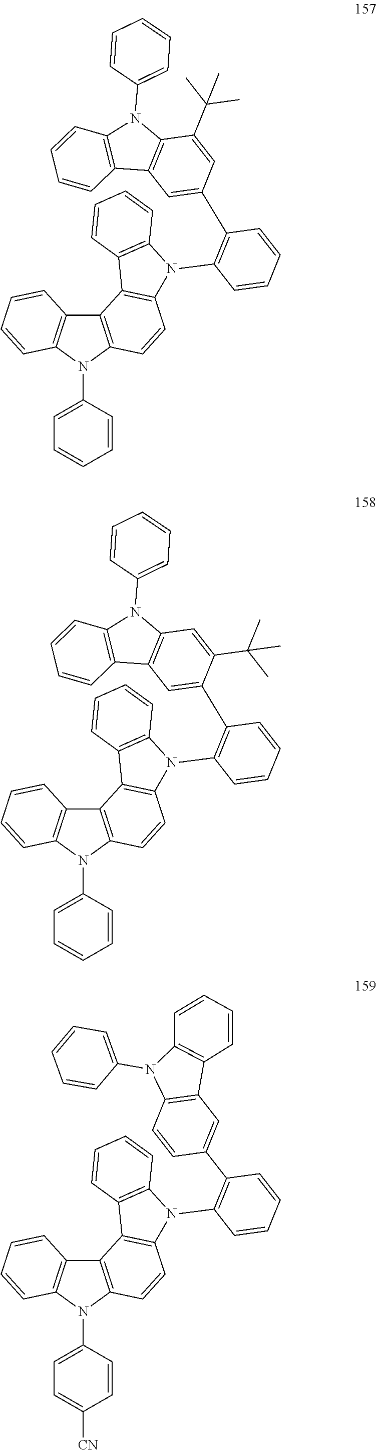 [SMILES: CC(C)(C)c1cc(-c2ccccc2-n2c3ccccc3c3c4c5ccccc5n(-c5ccccc5)c4ccc32)cc2c3ccccc3n(-c3ccccc3)c12 ; CC(C)(C)c1cc2c(cc1-c1ccccc1-n1c3ccccc3c3c4c5ccccc5n(-c5ccccc5)c4ccc31)c1ccccc1n2-c1ccccc1 ; N#Cc1ccc(-n2c3ccccc3c3c4c5ccccc5n(-c5ccccc5-c5ccc6c(c5)c5ccccc5n6-c5ccccc5)c4ccc32)cc1] 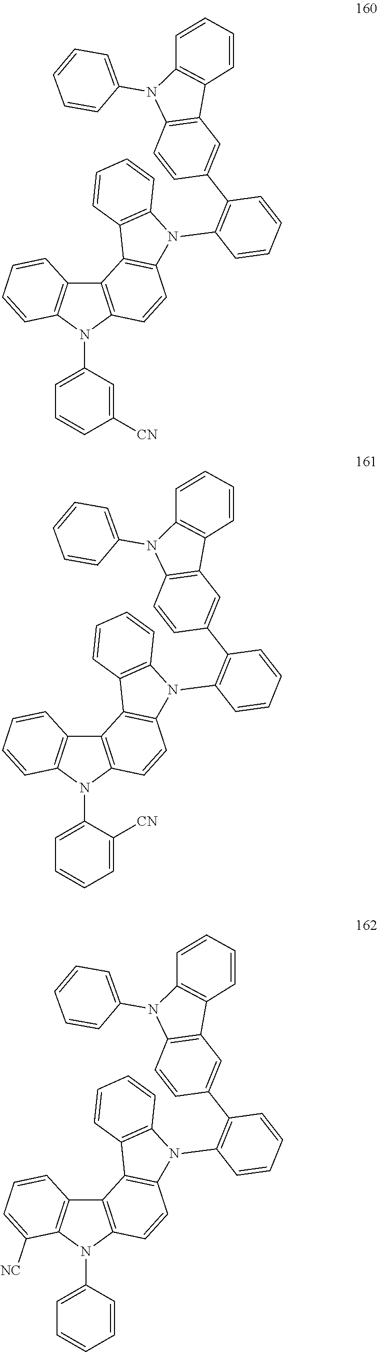 [SMILES: N#Cc1cccc(-n2c3ccccc3c3c4c5ccccc5n(-c5ccccc5-c5ccc6c(c5)c5ccccc5n6-c5ccccc5)c4ccc32)c1 ; N#Cc1ccccc1-n1c2ccccc2c2c3c4ccccc4n(-c4ccccc4-c4ccc5c(c4)c4ccccc4n5-c4ccccc4)c3ccc21 ; N#Cc1cccc2c3c4c5ccccc5n(-c5ccccc5-c5ccc6c(c5)c5ccccc5n6-c5ccccc5)c4ccc3n(-c3ccccc3)c12] 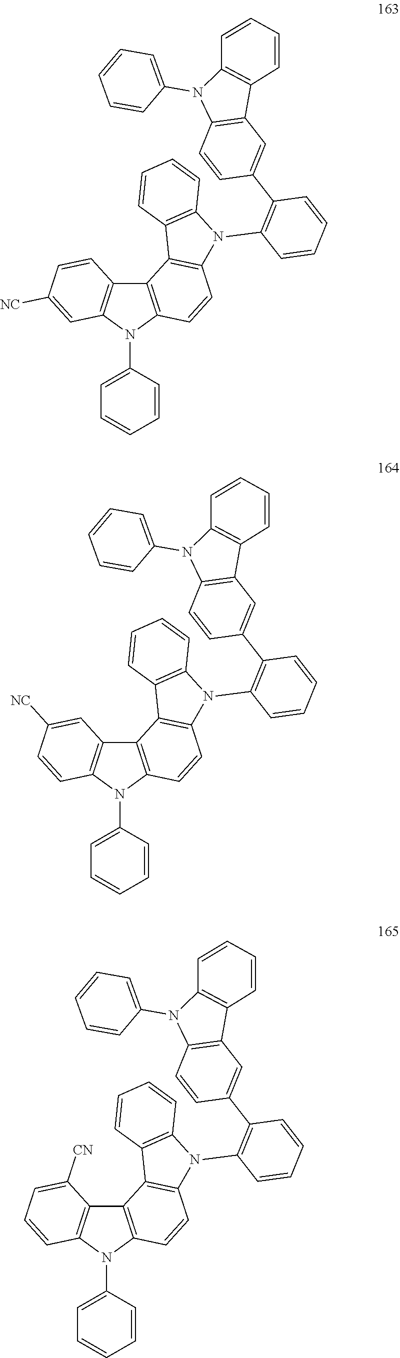 [SMILES: N#Cc1ccc2c3c4c5ccccc5n(-c5ccccc5-c5ccc6c(c5)c5ccccc5n6-c5ccccc5)c4ccc3n(-c3ccccc3)c2c1 ; N#Cc1ccc2c(c1)c1c3c4ccccc4n(-c4ccccc4-c4ccc5c(c4)c4ccccc4n5-c4ccccc4)c3ccc1n2-c1ccccc1 ; N#Cc1cccc2c1c1c3c4ccccc4n(-c4ccccc4-c4ccc5c(c4)c4ccccc4n5-c4ccccc4)c3ccc1n2-c1ccccc1] 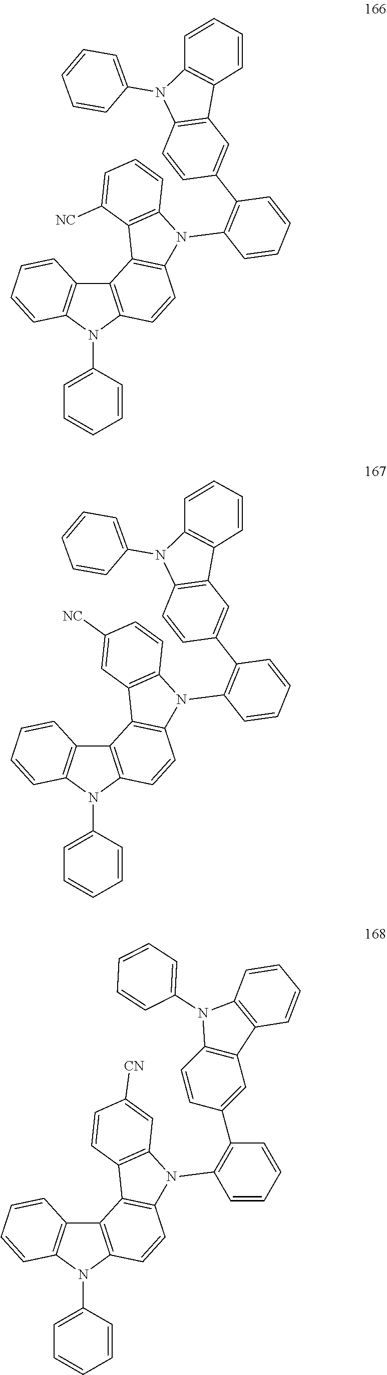 [SMILES: N#Cc1cccc2c1c1c3c4ccccc4n(-c4ccccc4)c3ccc1n2-c1ccccc1-c1ccc2c(c1)c1ccccc1n2-c1ccccc1 ; N#Cc1ccc2c(c1)c1c3c4ccccc4n(-c4ccccc4)c3ccc1n2-c1ccccc1-c1ccc2c(c1)c1ccccc1n2-c1ccccc1 ; N#Cc1ccc2c3c4c5ccccc5n(-c5ccccc5)c4ccc3n(-c3ccccc3-c3ccc4c(c3)c3ccccc3n4-c3ccccc3)c2c1] 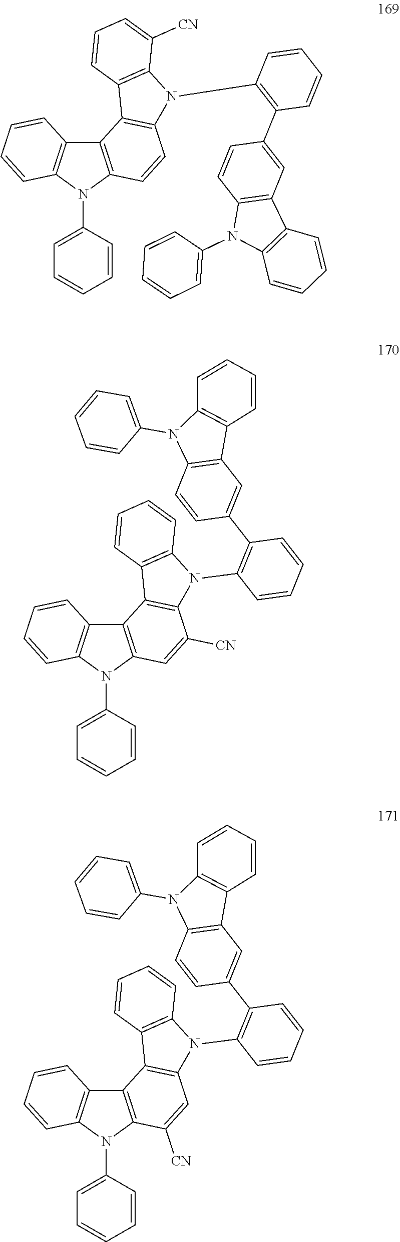 [SMILES: N#Cc1cccc2c3c4c5ccccc5n(-c5ccccc5)c4ccc3n(-c3ccccc3-c3ccc4c(c3)c3ccccc3n4-c3ccccc3)c12 ; N#Cc1cc2c(c3ccccc3n2-c2ccccc2)c2c3ccccc3n(-c3ccccc3-c3ccc4c(c3)c3ccccc3n4-c3ccccc3)c12 ; N#Cc1cc2c(c3ccccc3n2-c2ccccc2-c2ccc3c(c2)c2ccccc2n3-c2ccccc2)c2c3ccccc3n(-c3ccccc3)c12] 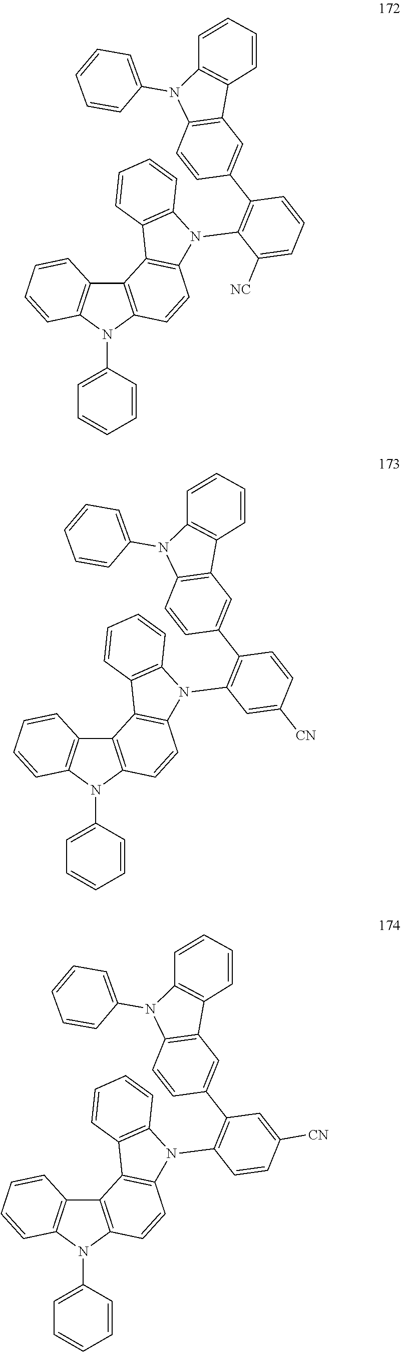 [SMILES: N#Cc1cccc(-c2ccc3c(c2)c2ccccc2n3-c2ccccc2)c1-n1c2ccccc2c2c3c4ccccc4n(-c4ccccc4)c3ccc21 ; N#Cc1ccc(-c2ccc3c(c2)c2ccccc2n3-c2ccccc2)c(-n2c3ccccc3c3c4c5ccccc5n(-c5ccccc5)c4ccc32)c1 ; N#Cc1ccc(-n2c3ccccc3c3c4c5ccccc5n(-c5ccccc5)c4ccc32)c(-c2ccc3c(c2)c2ccccc2n3-c2ccccc2)c1] 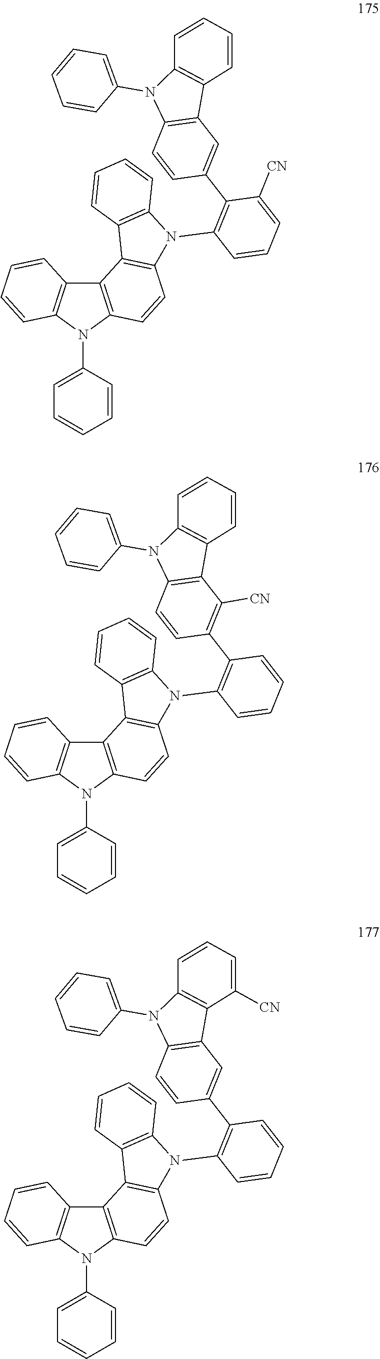 [SMILES: N#Cc1cccc(-n2c3ccccc3c3c4c5ccccc5n(-c5ccccc5)c4ccc32)c1-c1ccc2c(c1)c1ccccc1n2-c1ccccc1 ; N#Cc1c(-c2ccccc2-n2c3ccccc3c3c4c5ccccc5n(-c5ccccc5)c4ccc32)ccc2c1c1ccccc1n2-c1ccccc1 ; N#Cc1cccc2c1c1cc(-c3ccccc3-n3c4ccccc4c4c5c6ccccc6n(-c6ccccc6)c5ccc43)ccc1n2-c1ccccc1] 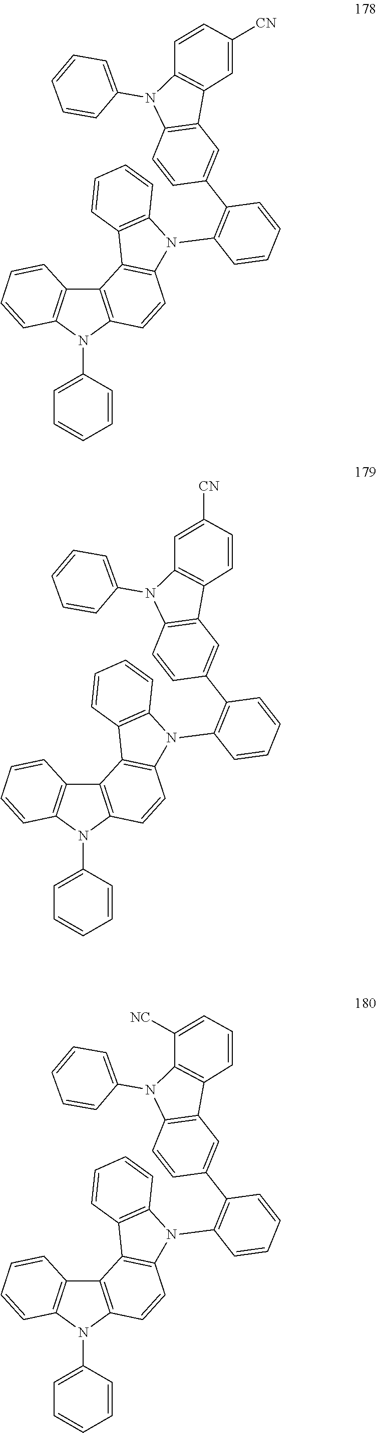 [SMILES: N#Cc1ccc2c(c1)c1cc(-c3ccccc3-n3c4ccccc4c4c5c6ccccc6n(-c6ccccc6)c5ccc43)ccc1n2-c1ccccc1 ; N#Cc1ccc2c3cc(-c4ccccc4-n4c5ccccc5c5c6c7ccccc7n(-c7ccccc7)c6ccc54)ccc3n(-c3ccccc3)c2c1 ; N#Cc1cccc2c3cc(-c4ccccc4-n4c5ccccc5c5c6c7ccccc7n(-c7ccccc7)c6ccc54)ccc3n(-c3ccccc3)c12] 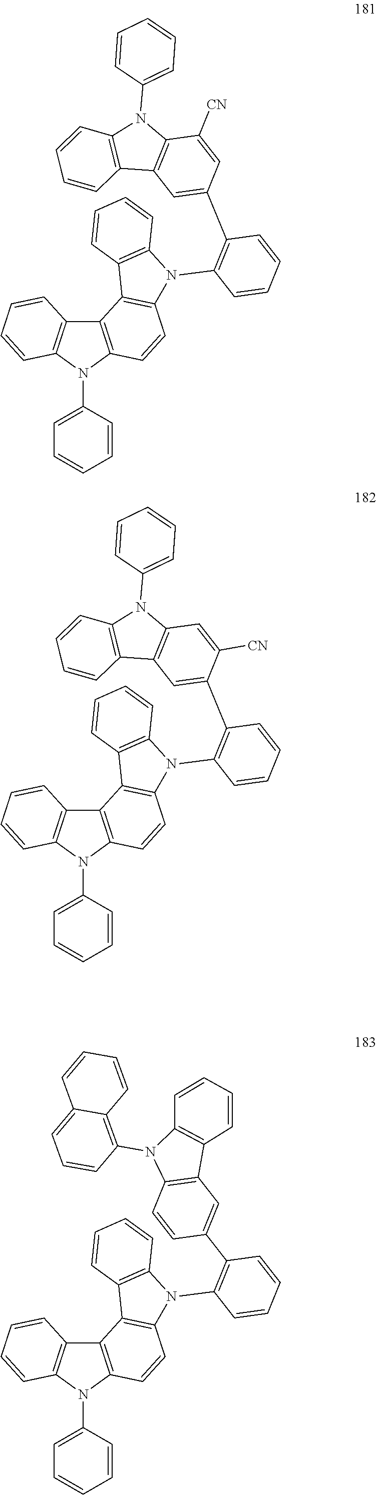 [SMILES: N#Cc1cc(-c2ccccc2-n2c3ccccc3c3c4c5ccccc5n(-c5ccccc5)c4ccc32)cc2c3ccccc3n(-c3ccccc3)c12 ; N#Cc1cc2c(cc1-c1ccccc1-n1c3ccccc3c3c4c5ccccc5n(-c5ccccc5)c4ccc31)c1ccccc1n2-c1ccccc1 ; c1ccc(-n2c3ccccc3c3c4c5ccccc5n(-c5ccccc5-c5ccc6c(c5)c5ccccc5n6-c5cccc6ccccc56)c4ccc32)cc1] 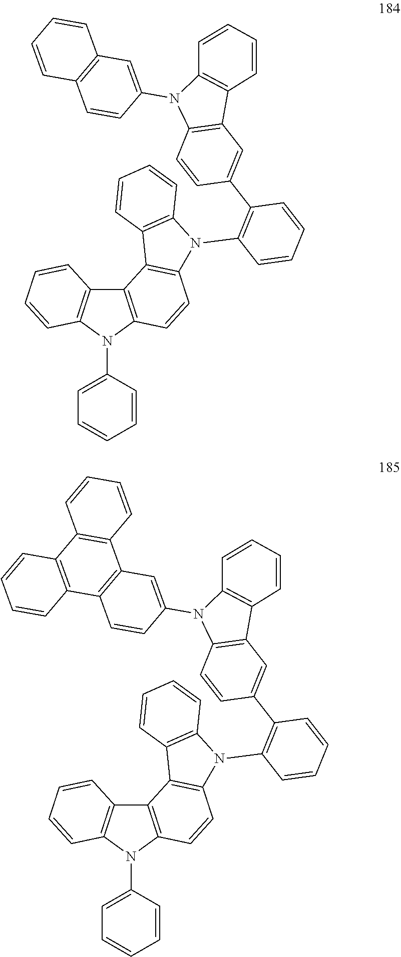 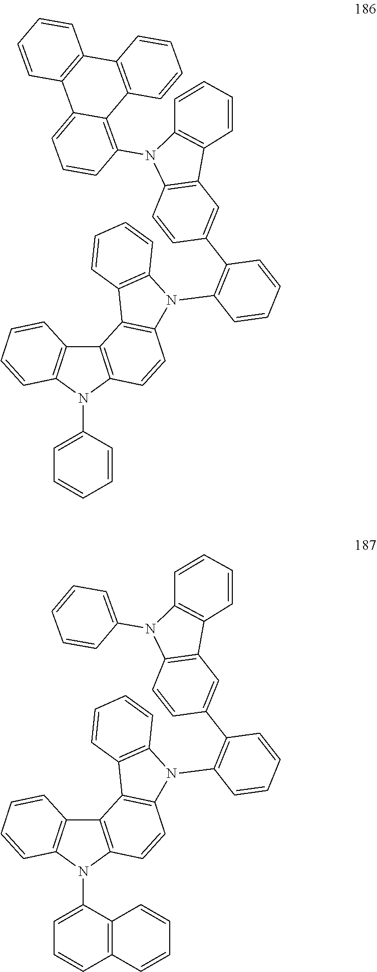 [SMILES: c1ccc(-n2c3ccccc3c3c4c5ccccc5n(-c5ccccc5-c5ccc6c(c5)c5ccccc5n6-c5cccc6c7ccccc7c7ccccc7c56)c4ccc32)cc1 ; c1ccc(-n2c3ccccc3c3cc(-c4ccccc4-n4c5ccccc5c5c6c7ccccc7n(-c7cccc8ccccc78)c6ccc54)ccc32)cc1] 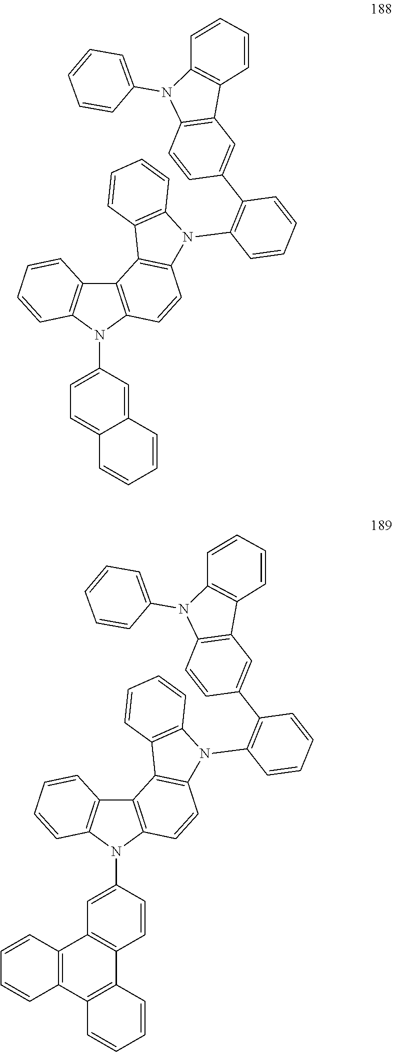 [SMILES: c1ccc(-n2c3ccccc3c3cc(-c4ccccc4-n4c5ccccc5c5c6c7ccccc7n(-c7ccc8ccccc8c7)c6ccc54)ccc32)cc1 ; c1ccc(-n2c3ccccc3c3cc(-c4ccccc4-n4c5ccccc5c5c6c7ccccc7n(-c7ccc8c9ccccc9c9ccccc9c8c7)c6ccc54)ccc32)cc1] 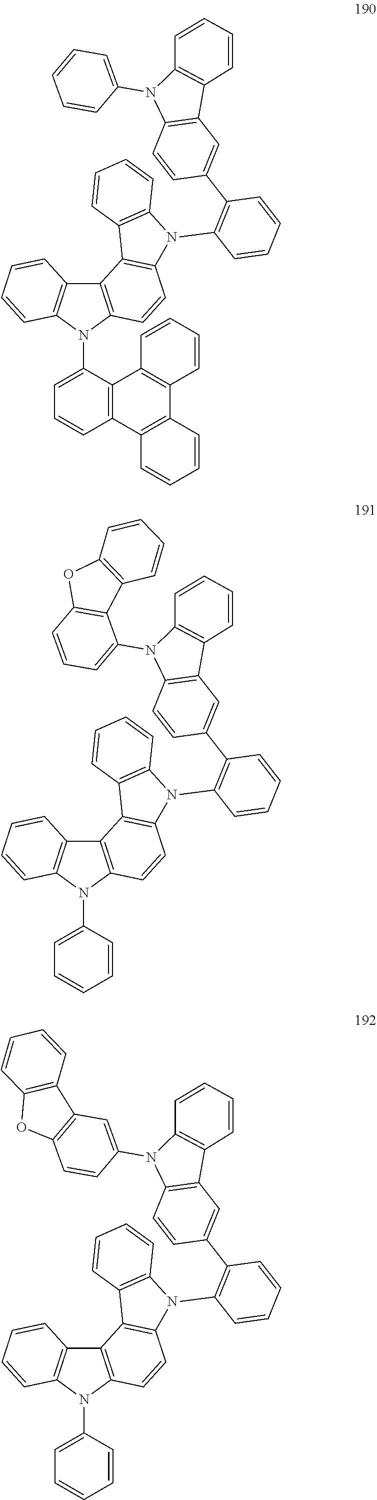 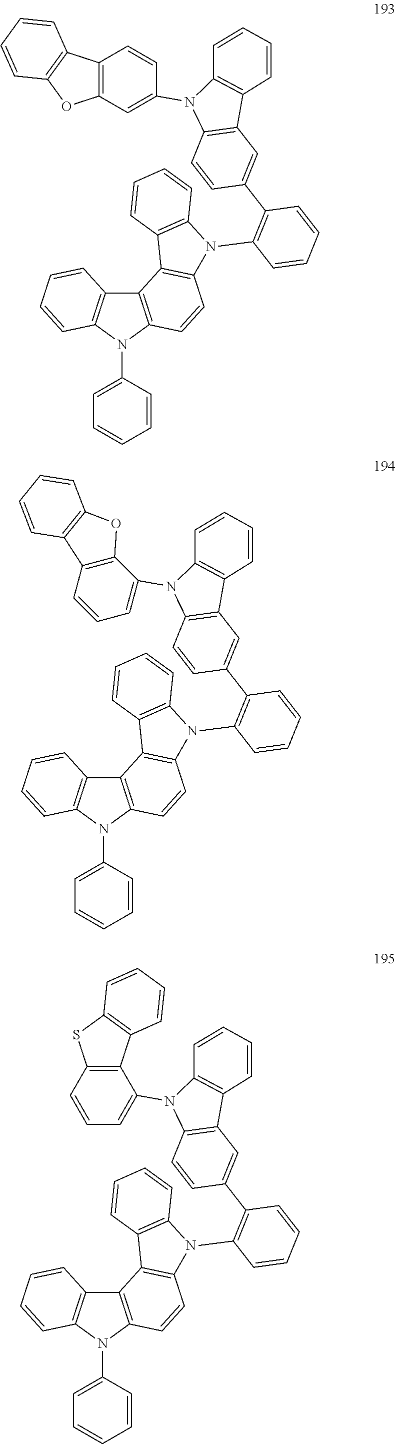 [SMILES: c1ccc(-n2c3ccccc3c3c4c5ccccc5n(-c5ccccc5-c5ccc6c(c5)c5ccccc5n6-c5ccc6c(c5)oc5ccccc56)c4ccc32)cc1 ; c1ccc(-n2c3ccccc3c3c4c5ccccc5n(-c5ccccc5-c5ccc6c(c5)c5ccccc5n6-c5cccc6c5oc5ccccc56)c4ccc32)cc1 ; c1ccc(-n2c3ccccc3c3c4c5ccccc5n(-c5ccccc5-c5ccc6c(c5)c5ccccc5n6-c5cccc6sc7ccccc7c56)c4ccc32)cc1] 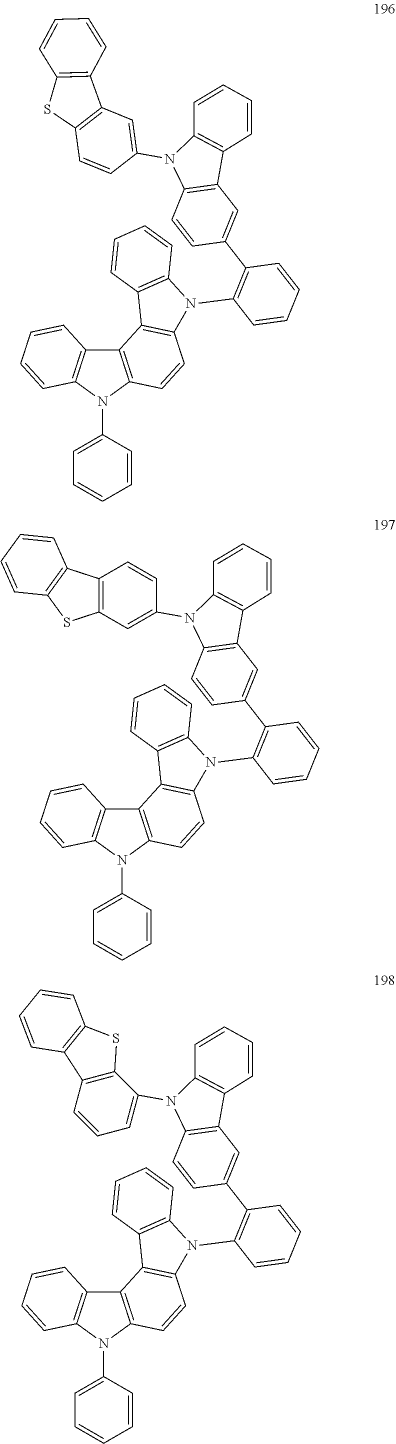 [SMILES: c1ccc(-n2c3ccccc3c3c4c5ccccc5n(-c5ccccc5-c5ccc6c(c5)c5ccccc5n6-c5ccc6sc7ccccc7c6c5)c4ccc32)cc1 ; c1ccc(-n2c3ccccc3c3c4c5ccccc5n(-c5ccccc5-c5ccc6c(c5)c5ccccc5n6-c5ccc6c(c5)sc5ccccc56)c4ccc32)cc1 ; c1ccc(-n2c3ccccc3c3c4c5ccccc5n(-c5ccccc5-c5ccc6c(c5)c5ccccc5n6-c5cccc6c5sc5ccccc56)c4ccc32)cc1] 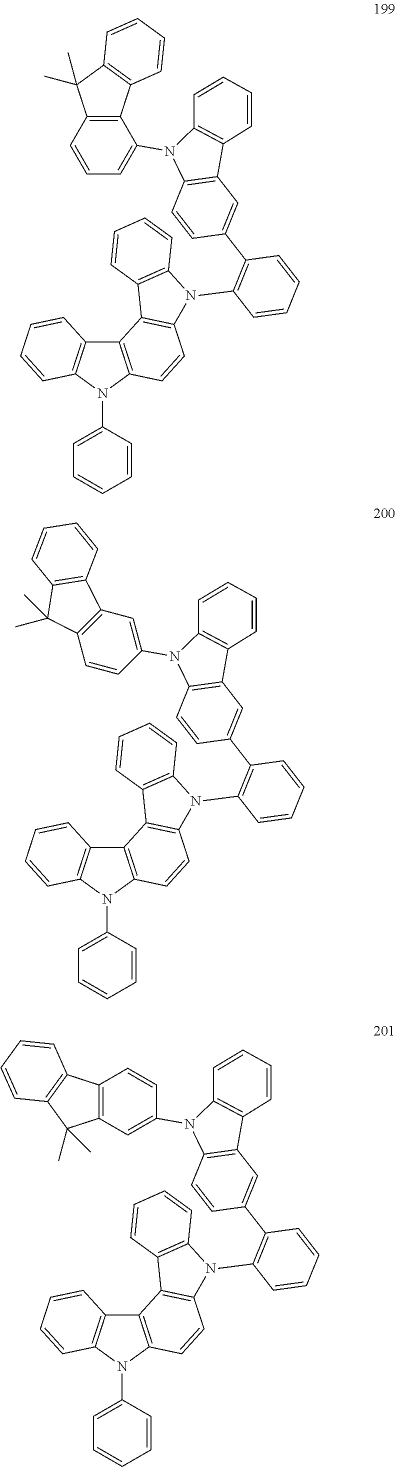 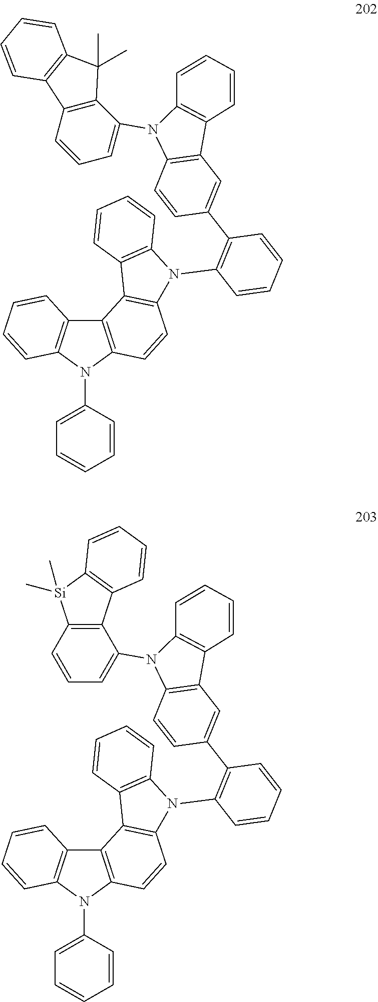 [SMILES: CC1(C)c2ccccc2-c2cccc(-n3c4ccccc4c4cc(-c5ccccc5-n5c6ccccc6c6c7c8ccccc8n(-c8ccccc8)c7ccc65)ccc43)c21 ; C[Si]1(C)c2ccccc2-c2c(-n3c4ccccc4c4cc(-c5ccccc5-n5c6ccccc6c6c7c8ccccc8n(-c8ccccc8)c7ccc65)ccc43)cccc21] 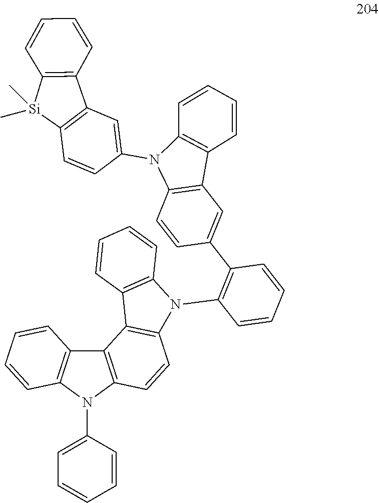 [SMILES: C[Si]1(C)c2ccccc2-c2cc(-n3c4ccccc4c4cc(-c5ccccc5-n5c6ccccc6c6c7c8ccccc8n(-c8ccccc8)c7ccc65)ccc43)ccc21] 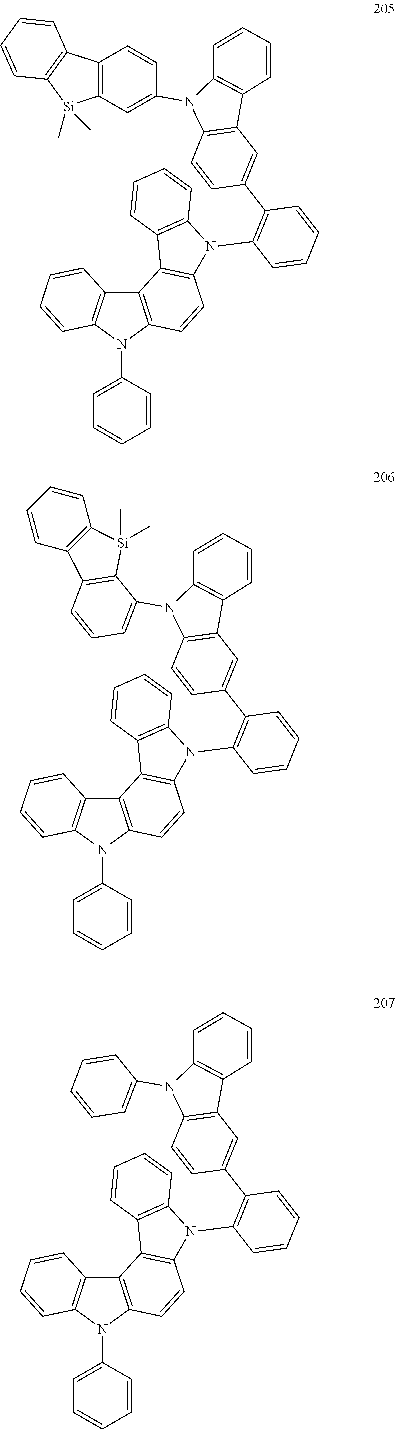 [SMILES: C[Si]1(C)c2ccccc2-c2ccc(-n3c4ccccc4c4cc(-c5ccccc5-n5c6ccccc6c6c7c8ccccc8n(-c8ccccc8)c7ccc65)ccc43)cc21 ; C[Si]1(C)c2ccccc2-c2cccc(-n3c4ccccc4c4cc(-c5ccccc5-n5c6ccccc6c6c7c8ccccc8n(-c8ccccc8)c7ccc65)ccc43)c21 ; c1ccc(-n2c3ccccc3c3cc(-c4ccccc4-n4c5ccccc5c5c6c7ccccc7n(-c7ccccc7)c6ccc54)ccc32)cc1] 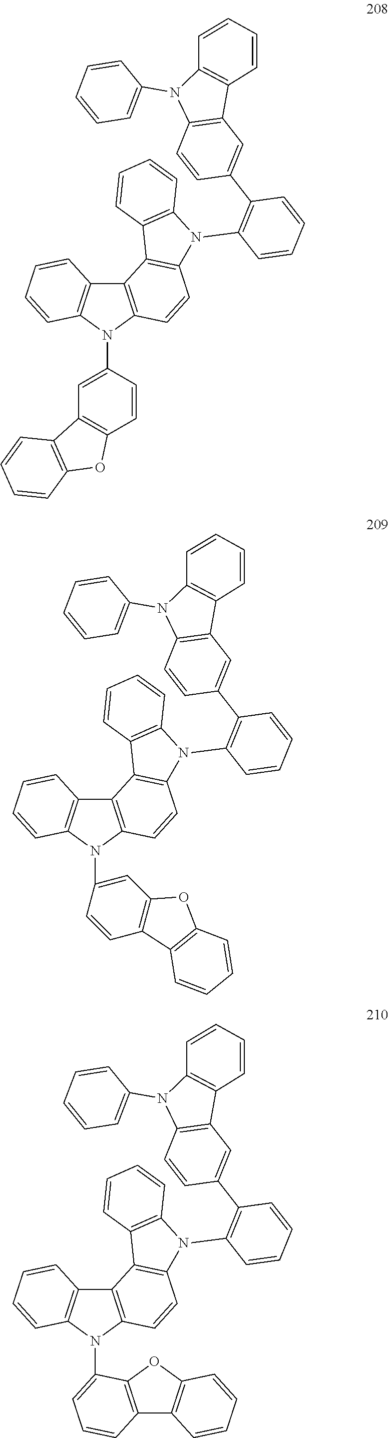 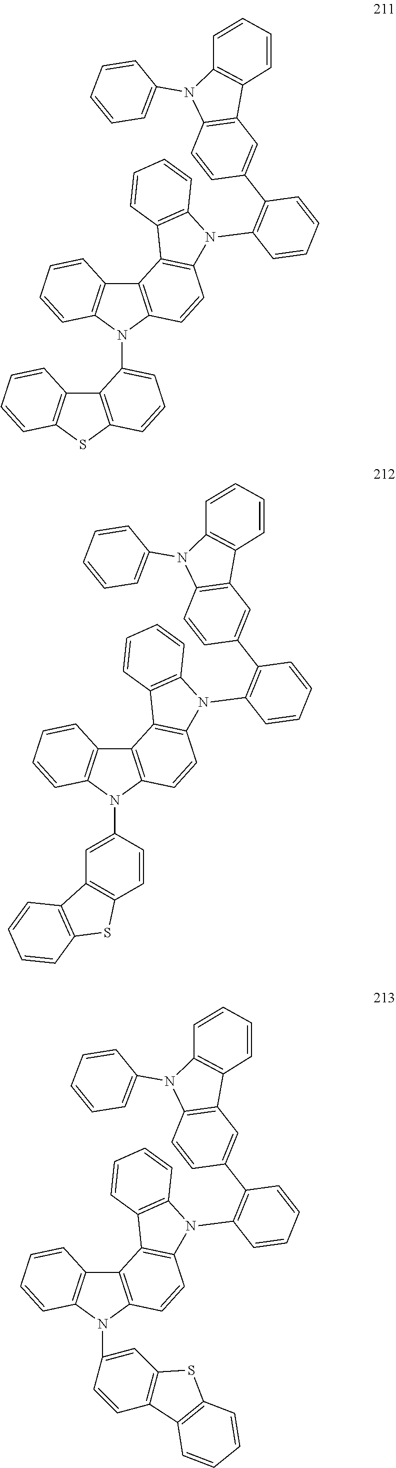 [SMILES: c1ccc(-n2c3ccccc3c3cc(-c4ccccc4-n4c5ccccc5c5c6c7ccccc7n(-c7cccc8sc9ccccc9c78)c6ccc54)ccc32)cc1 ; c1ccc(-n2c3ccccc3c3cc(-c4ccccc4-n4c5ccccc5c5c6c7ccccc7n(-c7ccc8sc9ccccc9c8c7)c6ccc54)ccc32)cc1 ; c1ccc(-n2c3ccccc3c3cc(-c4ccccc4-n4c5ccccc5c5c6c7ccccc7n(-c7ccc8c(c7)sc7ccccc78)c6ccc54)ccc32)cc1] 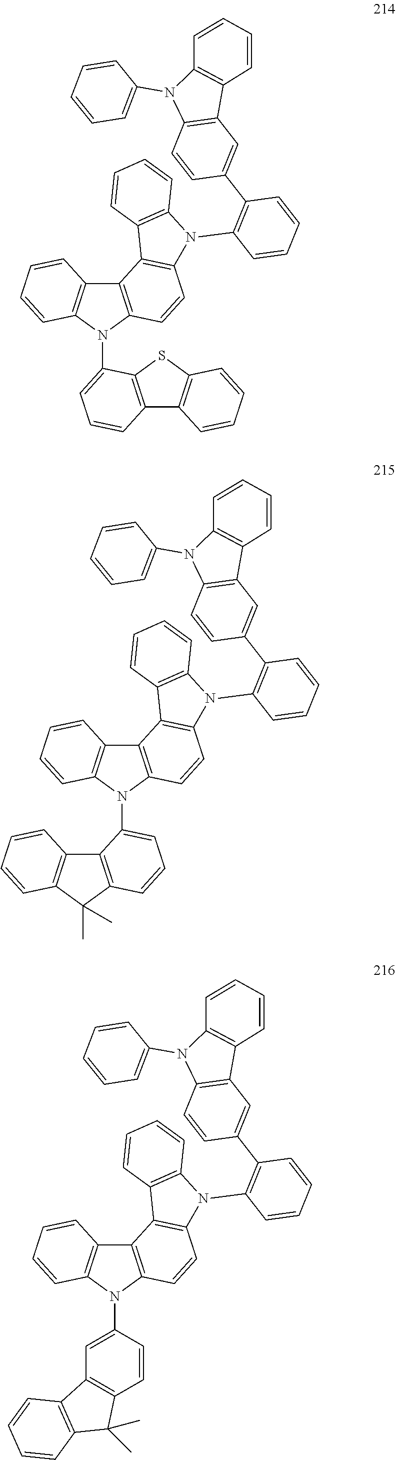 [SMILES: c1ccc(-n2c3ccccc3c3cc(-c4ccccc4-n4c5ccccc5c5c6c7ccccc7n(-c7cccc8c7sc7ccccc78)c6ccc54)ccc32)cc1 ; CC1(C)c2ccccc2-c2c(-n3c4ccccc4c4c5c6ccccc6n(-c6ccccc6-c6ccc7c(c6)c6ccccc6n7-c6ccccc6)c5ccc43)cccc21 ; CC1(C)c2ccccc2-c2cc(-n3c4ccccc4c4c5c6ccccc6n(-c6ccccc6-c6ccc7c(c6)c6ccccc6n7-c6ccccc6)c5ccc43)ccc21] 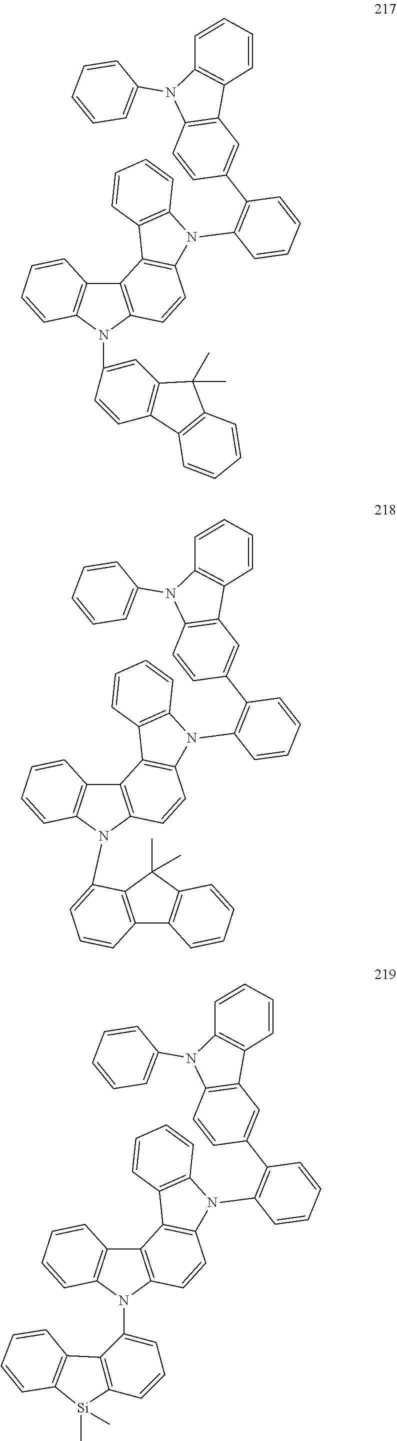 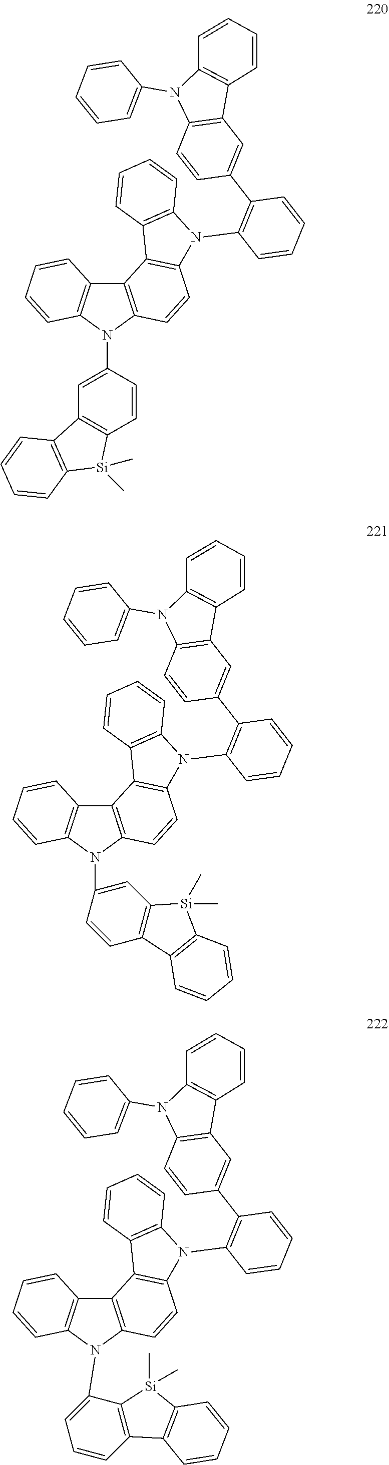 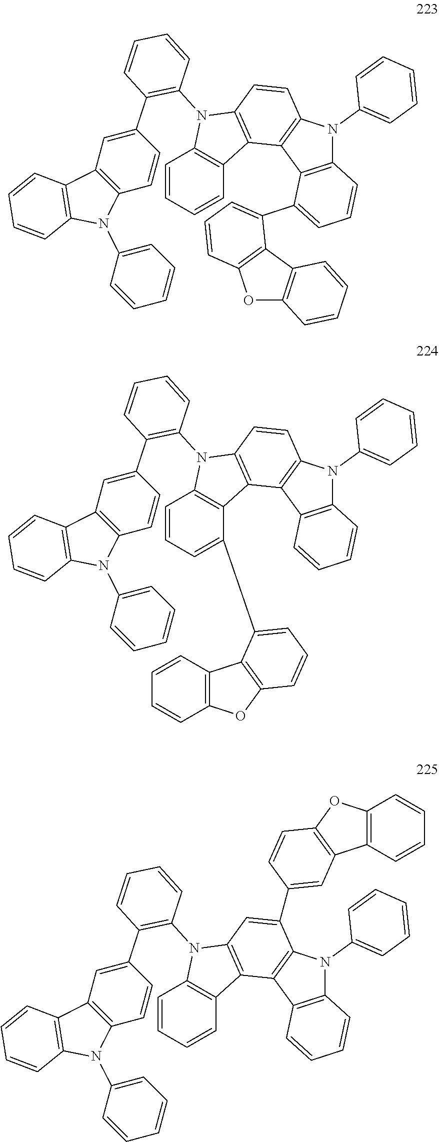 [SMILES: c1ccc(-n2c3ccccc3c3cc(-c4ccccc4-n4c5ccccc5c5c6c7c(-c8cccc9oc%10ccccc%10c89)cccc7n(-c7ccccc7)c6ccc54)ccc32)cc1 ; c1ccc(-n2c3ccccc3c3cc(-c4ccccc4-n4c5cccc(-c6cccc7oc8ccccc8c67)c5c5c6c7ccccc7n(-c7ccccc7)c6ccc54)ccc32)cc1 ; c1ccc(-n2c3ccccc3c3cc(-c4ccccc4-n4c5ccccc5c5c6c7ccccc7n(-c7ccccc7)c6c(-c6ccc7oc8ccccc8c7c6)cc54)ccc32)cc1] 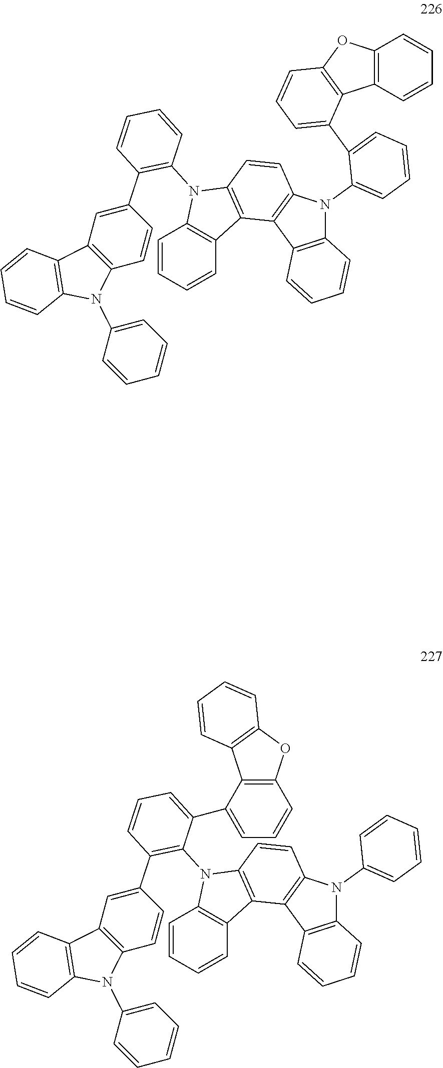 [SMILES: c1ccc(-n2c3ccccc3c3cc(-c4ccccc4-n4c5ccccc5c5c6c7ccccc7n(-c7ccccc7-c7cccc8oc9ccccc9c78)c6ccc54)ccc32)cc1 ; c1ccc(-n2c3ccccc3c3cc(-c4cccc(-c5cccc6oc7ccccc7c56)c4-n4c5ccccc5c5c6c7ccccc7n(-c7ccccc7)c6ccc54)ccc32)cc1] 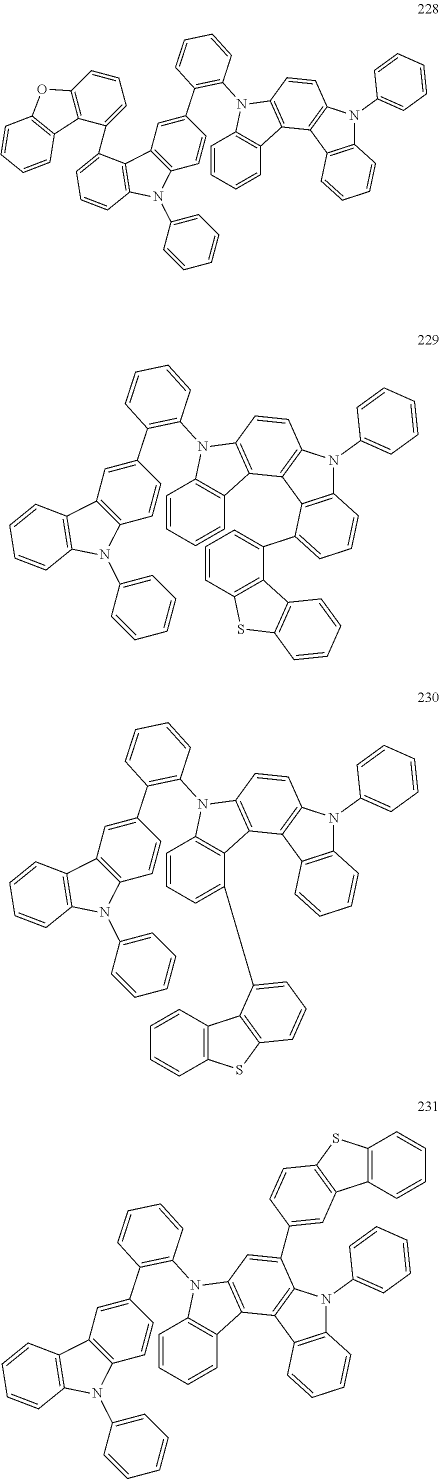 [SMILES: c1ccc(-n2c3ccc(-c4ccccc4-n4c5ccccc5c5c6c7ccccc7n(-c7ccccc7)c6ccc54)cc3c3c(-c4cccc5oc6ccccc6c45)cccc32)cc1 ; c1ccc(-n2c3ccccc3c3cc(-c4ccccc4-n4c5ccccc5c5c6c7c(-c8cccc9sc%10ccccc%10c89)cccc7n(-c7ccccc7)c6ccc54)ccc32)cc1 ; c1ccc(-n2c3ccccc3c3cc(-c4ccccc4-n4c5cccc(-c6cccc7sc8ccccc8c67)c5c5c6c7ccccc7n(-c7ccccc7)c6ccc54)ccc32)cc1 ; c1ccc(-n2c3ccccc3c3cc(-c4ccccc4-n4c5ccccc5c5c6c7ccccc7n(-c7ccccc7)c6c(-c6ccc7sc8ccccc8c7c6)cc54)ccc32)cc1] 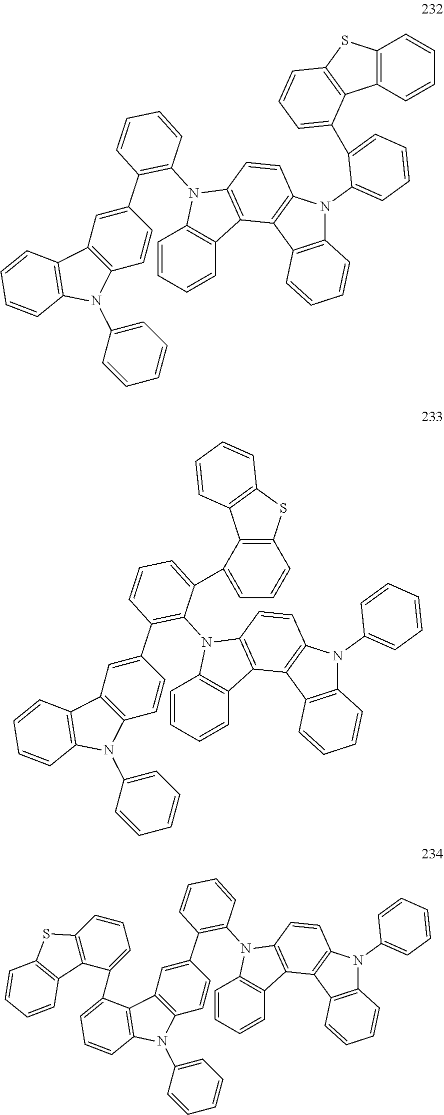 [SMILES: c1ccc(-n2c3ccccc3c3cc(-c4ccccc4-n4c5ccccc5c5c6c7ccccc7n(-c7ccccc7-c7cccc8sc9ccccc9c78)c6ccc54)ccc32)cc1 ; c1ccc(-n2c3ccccc3c3cc(-c4cccc(-c5cccc6sc7ccccc7c56)c4-n4c5ccccc5c5c6c7ccccc7n(-c7ccccc7)c6ccc54)ccc32)cc1 ; c1ccc(-n2c3ccc(-c4ccccc4-n4c5ccccc5c5c6c7ccccc7n(-c7ccccc7)c6ccc54)cc3c3c(-c4cccc5sc6ccccc6c45)cccc32)cc1] 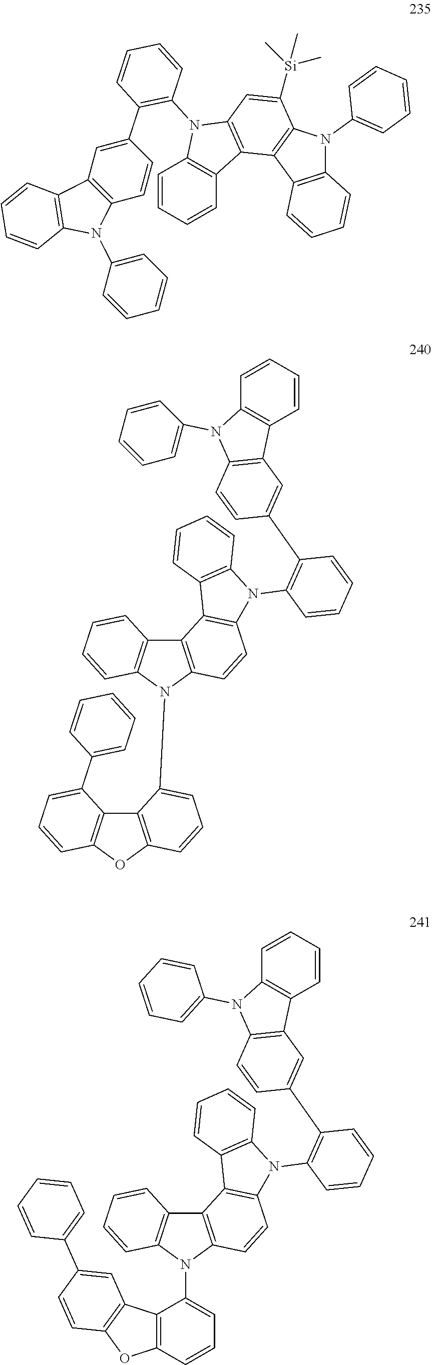 [SMILES: C[Si](C)(C)c1cc2c(c3ccccc3n2-c2ccccc2-c2ccc3c(c2)c2ccccc2n3-c2ccccc2)c2c3ccccc3n(-c3ccccc3)c12 ; c1ccc(-c2cccc3oc4cccc(-n5c6ccccc6c6c7c8ccccc8n(-c8ccccc8-c8ccc9c(c8)c8ccccc8n9-c8ccccc8)c7ccc65)c4c23)cc1 ; c1ccc(-c2ccc3oc4cccc(-n5c6ccccc6c6c7c8ccccc8n(-c8ccccc8-c8ccc9c(c8)c8ccccc8n9-c8ccccc8)c7ccc65)c4c3c2)cc1] 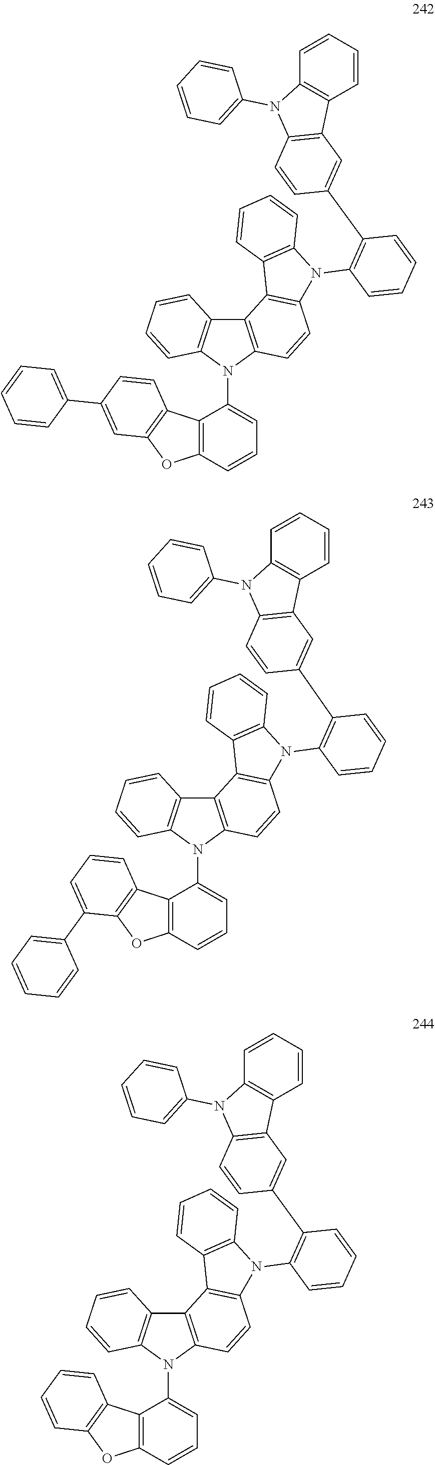 [SMILES: c1ccc(-c2ccc3c(c2)oc2cccc(-n4c5ccccc5c5c6c7ccccc7n(-c7ccccc7-c7ccc8c(c7)c7ccccc7n8-c7ccccc7)c6ccc54)c23)cc1 ; c1ccc(-c2cccc3c2oc2cccc(-n4c5ccccc5c5c6c7ccccc7n(-c7ccccc7-c7ccc8c(c7)c7ccccc7n8-c7ccccc7)c6ccc54)c23)cc1 ; c1ccc(-n2c3ccccc3c3cc(-c4ccccc4-n4c5ccccc5c5c6c7ccccc7n(-c7cccc8oc9ccccc9c78)c6ccc54)ccc32)cc1] 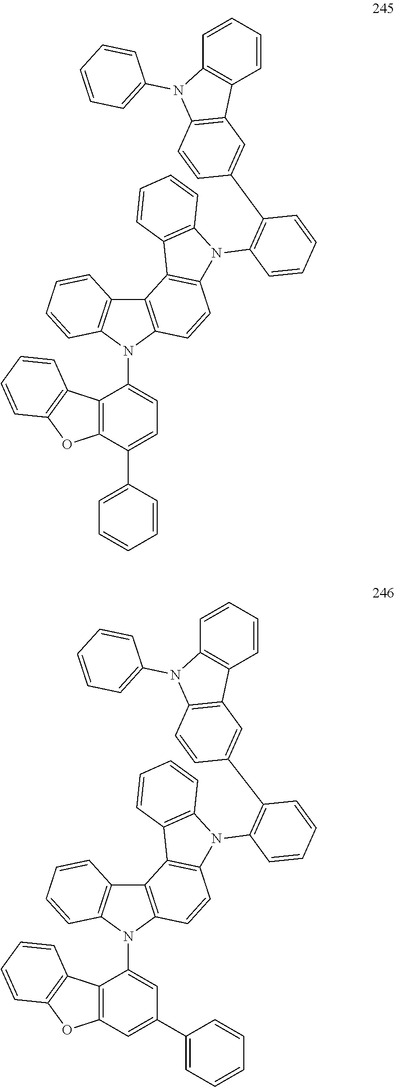 [SMILES: c1ccc(-c2ccc(-n3c4ccccc4c4c5c6ccccc6n(-c6ccccc6-c6ccc7c(c6)c6ccccc6n7-c6ccccc6)c5ccc43)c3c2oc2ccccc23)cc1 ; c1ccc(-c2cc(-n3c4ccccc4c4c5c6ccccc6n(-c6ccccc6-c6ccc7c(c6)c6ccccc6n7-c6ccccc6)c5ccc43)c3c(c2)oc2ccccc23)cc1] 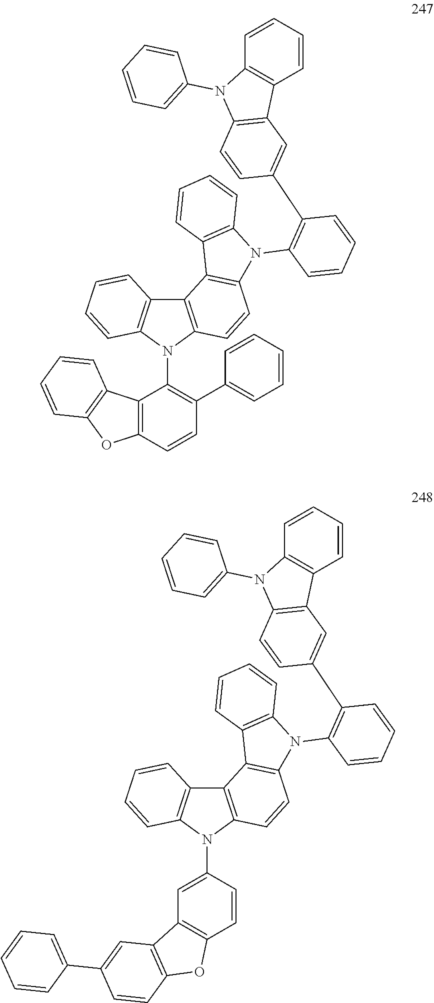 [SMILES: c1ccc(-c2ccc3oc4ccccc4c3c2-n2c3ccccc3c3c4c5ccccc5n(-c5ccccc5-c5ccc6c(c5)c5ccccc5n6-c5ccccc5)c4ccc32)cc1 ; c1ccc(-c2ccc3oc4ccc(-n5c6ccccc6c6c7c8ccccc8n(-c8ccccc8-c8ccc9c(c8)c8ccccc8n9-c8ccccc8)c7ccc65)cc4c3c2)cc1] 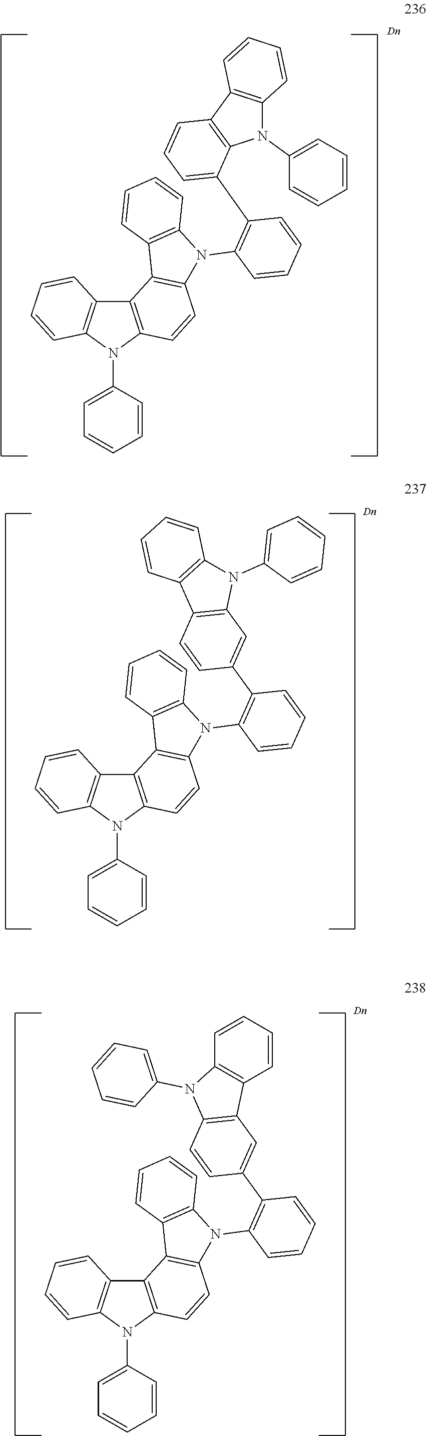 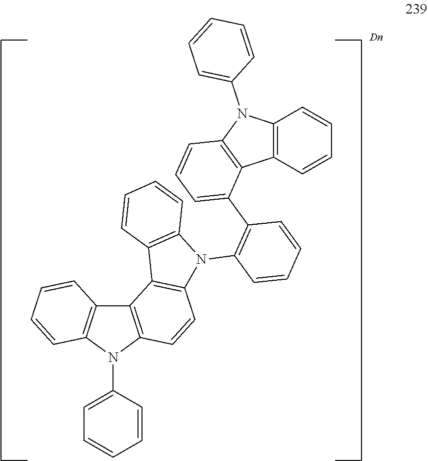
in Group 1, Dn refers to a number of deuterium substitutions and indicates a structure substituted with one or more deuterium atoms.

7. The compound for an organic optoelectronic device as claimed in claim 6, wherein:
the compound 238 is represented by one of compound of Group 1-1: 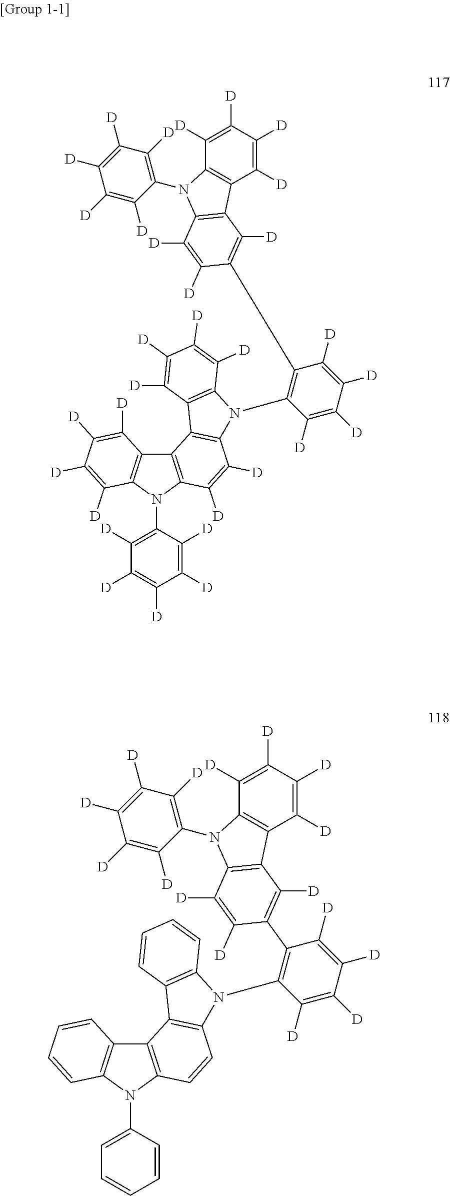 [SMILES: [2H]c1c([2H])c([2H])c(-n2c3c([2H])c([2H])c([2H])c([2H])c3c3c([2H])c(-c4c([2H])c([2H])c([2H])c([2H])c4-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c([2H])c32)c([2H])c1[2H] ; [2H]c1c([2H])c([2H])c(-n2c3c([2H])c([2H])c([2H])c([2H])c3c3c([2H])c(-c4c([2H])c([2H])c([2H])c([2H])c4-n4c5ccccc5c5c6c7ccccc7n(-c7ccccc7)c6ccc54)c([2H])c([2H])c32)c([2H])c1[2H]] 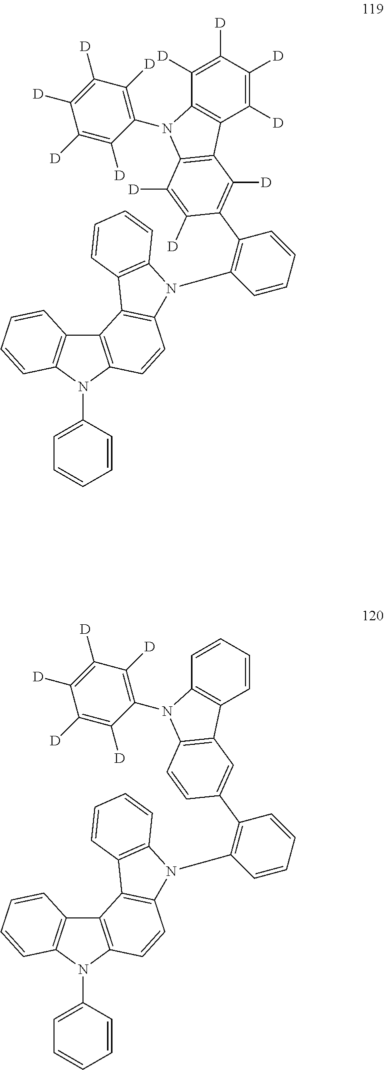 [SMILES: [2H]c1c([2H])c([2H])c(-n2c3c([2H])c([2H])c([2H])c([2H])c3c3c([2H])c(-c4ccccc4-n4c5ccccc5c5c6c7ccccc7n(-c7ccccc7)c6ccc54)c([2H])c([2H])c32)c([2H])c1[2H] ; [2H]c1c([2H])c([2H])c(-n2c3ccccc3c3cc(-c4ccccc4-n4c5ccccc5c5c6c7ccccc7n(-c7ccccc7)c6ccc54)ccc32)c([2H])c1[2H]] 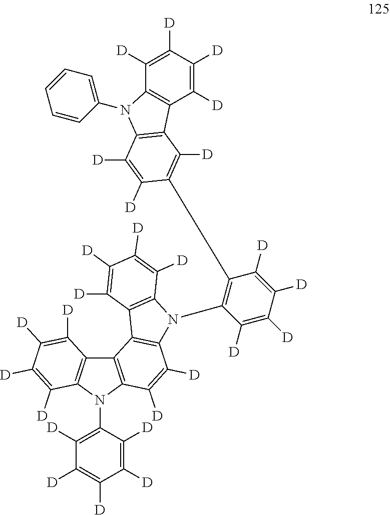 [SMILES: [2H]c1c([2H])c([2H])c(-n2c3c([2H])c([2H])c([2H])c([2H])c3c3c4c5c([2H])c([2H])c([2H])c([2H])c5n(-c5c([2H])c([2H])c([2H])c([2H])c5-c5c([2H])c([2H])c6c(c5[2H])c5c([2H])c([2H])c([2H])c([2H])c5n6-c5ccccc5)c4c([2H])c([2H])c32)c([2H])c1[2H]] 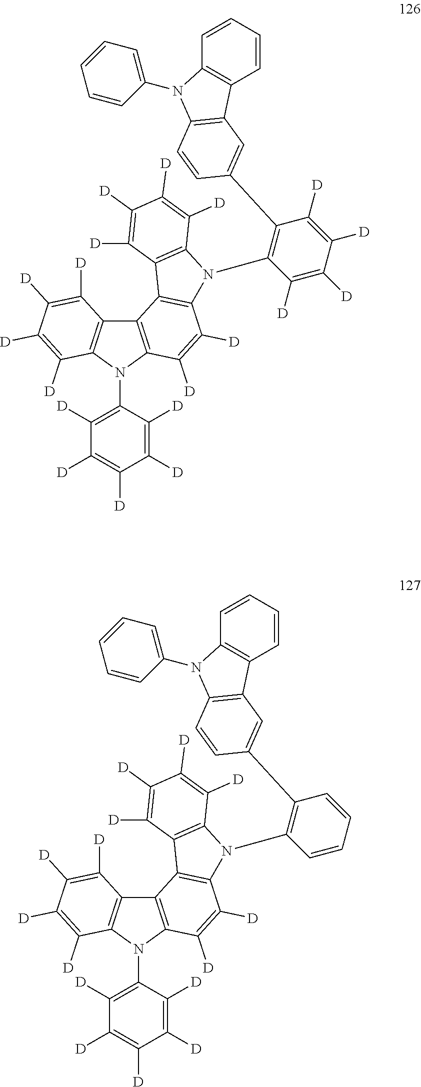 [SMILES: [2H]c1c([2H])c([2H])c(-n2c3c([2H])c([2H])c([2H])c([2H])c3c3c4c5c([2H])c([2H])c([2H])c([2H])c5n(-c5c([2H])c([2H])c([2H])c([2H])c5-c5ccc6c(c5)c5ccccc5n6-c5ccccc5)c4c([2H])c([2H])c32)c([2H])c1[2H] ; [2H]c1c([2H])c([2H])c(-n2c3c([2H])c([2H])c([2H])c([2H])c3c3c4c5c([2H])c([2H])c([2H])c([2H])c5n(-c5ccccc5-c5ccc6c(c5)c5ccccc5n6-c5ccccc5)c4c([2H])c([2H])c32)c([2H])c1[2H]] 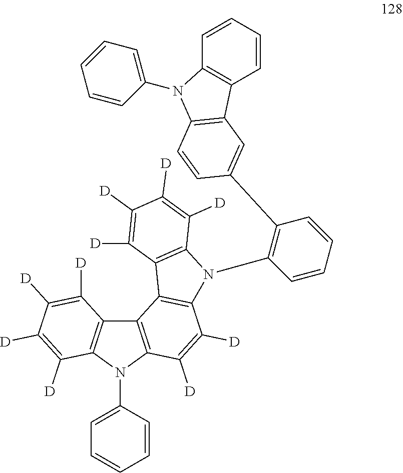 [SMILES: [2H]c1c([2H])c([2H])c2c(c1[2H])c1c3c4c([2H])c([2H])c([2H])c([2H])c4n(-c4ccccc4-c4ccc5c(c4)c4ccccc4n5-c4ccccc4)c3c([2H])c([2H])c1n2-c1ccccc1] 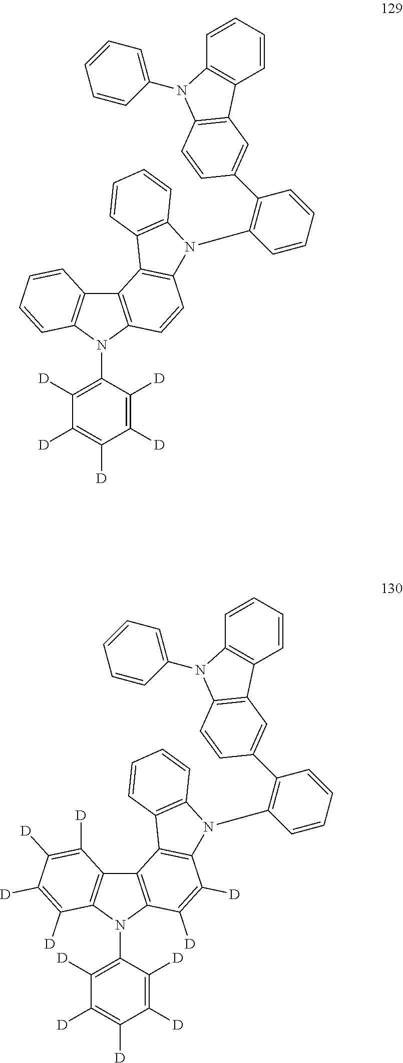 [SMILES: [2H]c1c([2H])c([2H])c(-n2c3ccccc3c3c4c5ccccc5n(-c5ccccc5-c5ccc6c(c5)c5ccccc5n6-c5ccccc5)c4ccc32)c([2H])c1[2H] ; [2H]c1c([2H])c([2H])c(-n2c3c([2H])c([2H])c([2H])c([2H])c3c3c4c5ccccc5n(-c5ccccc5-c5ccc6c(c5)c5ccccc5n6-c5ccccc5)c4c([2H])c([2H])c32)c([2H])c1[2H]] 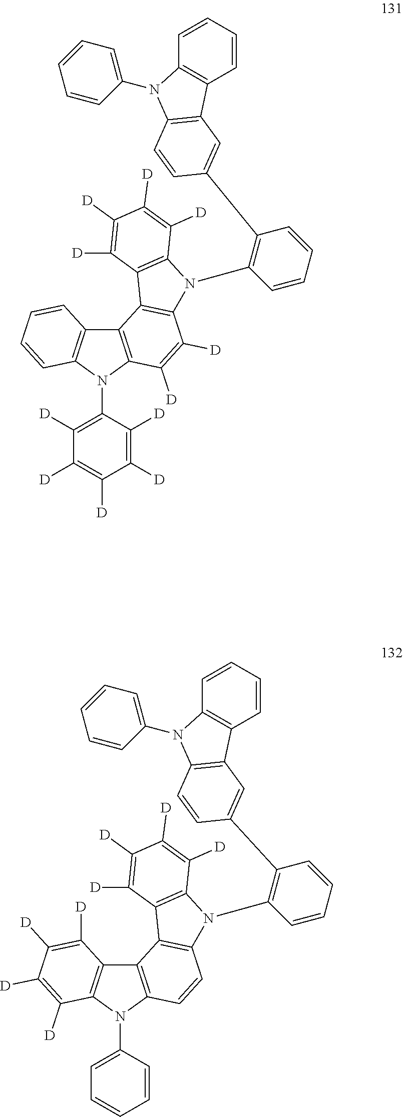 [SMILES: [2H]c1c([2H])c([2H])c(-n2c3ccccc3c3c4c5c([2H])c([2H])c([2H])c([2H])c5n(-c5ccccc5-c5ccc6c(c5)c5ccccc5n6-c5ccccc5)c4c([2H])c([2H])c32)c([2H])c1[2H] ; [2H]c1c([2H])c([2H])c2c(c1[2H])c1c3c4c([2H])c([2H])c([2H])c([2H])c4n(-c4ccccc4-c4ccc5c(c4)c4ccccc4n5-c4ccccc4)c3ccc1n2-c1ccccc1] 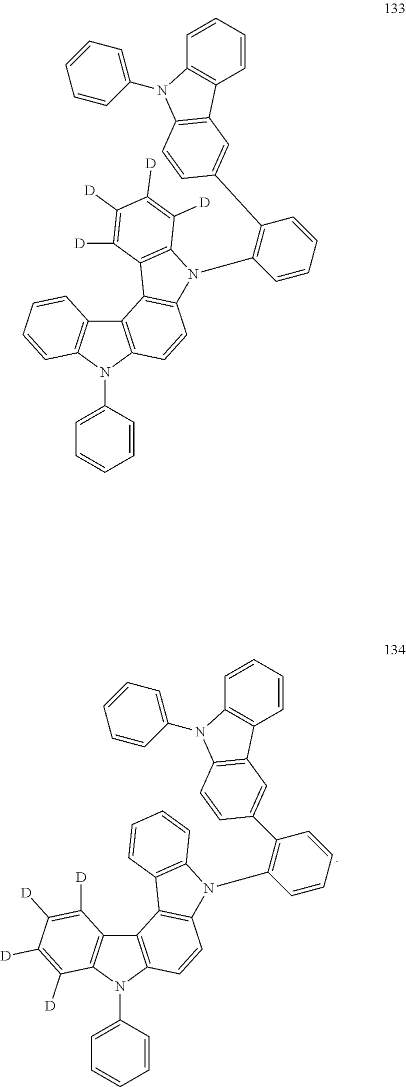 [SMILES: [2H]c1c([2H])c([2H])c2c(c1[2H])c1c3c4ccccc4n(-c4ccccc4)c3ccc1n2-c1ccccc1-c1ccc2c(c1)c1ccccc1n2-c1ccccc1 ; [2H]c1c([2H])c([2H])c2c(c1[2H])c1c3c4ccccc4n(-c4ccccc4-c4ccc5c(c4)c4ccccc4n5-c4ccccc4)c3ccc1n2-c1ccccc1]
in Group 1, Dn refers to a number of deuterium substitutions and indicates a structure substituted with one or more deuterium atoms.

8. A composition for an organic optoelectronic device, the composition comprising:
a first compound; and
a second compound,
wherein:
the first compound is the compound for an organic optoelectronic device as claimed in claim 1, and
the second compound is represented by Chemical Formula 2: 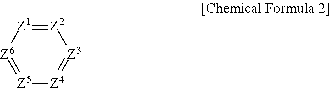
in Chemical Formula 2,
Z¹ to Z⁶ are each independently N or C-L^{a}-R^{a}, provided that at least two of Z¹ to Z⁶ are N,
each L^{a} is independently a single bond, a substituted or unsubstituted C6 to C20 arylene group, a substituted or unsubstituted C2 to C20 heterocyclic group, or a combination thereof,
each R^{a} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and
each R^{a} is separately present, or adjacent groups thereof are linked to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring.

9. The composition for an organic optoelectronic device as claimed in claim 8, wherein:
Chemical Formula 2 is represented by one of Chemical Formula 2A to Chemical Formula 2C: 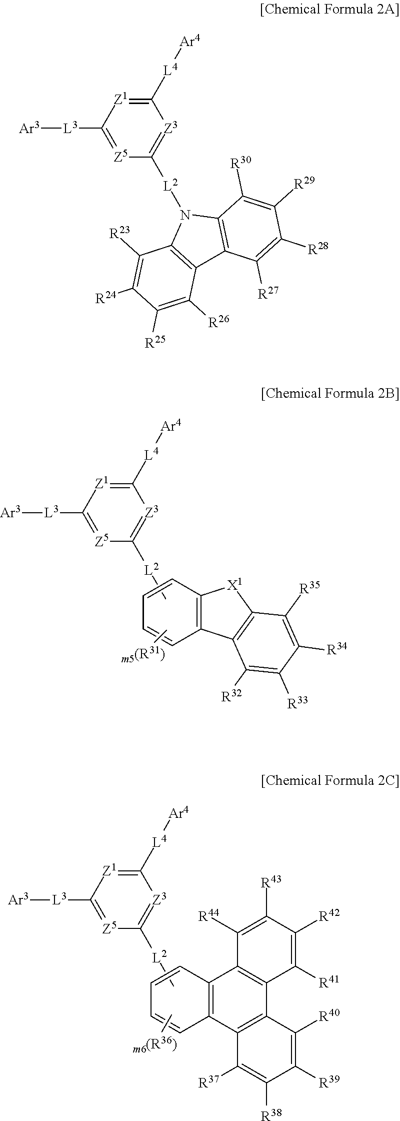
in Chemical Formulas 2A to 2C,
Z¹, Z³ and Z⁵ are each independently N or C-L^{a}-R^{a}, provided that at least two of Z¹, Z³, and Z⁵ are N,
X¹ is O, S, or NR^{b},
L^{a} and L² to L⁴ are each independently a single bond, a substituted or unsubstituted C6 to C20 arylene group, a substituted or unsubstituted C2 to C20 heterocyclic group, or a combination thereof,
R^{a}, R^{b}, and R²³ to R⁴⁴ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
R²³ to R³⁰ are each separately present or adjacent groups thereof are linked to form a substituted or unsubstituted aromatic monocyclic or polycyclic ring,
R³¹ to R³⁵ are each separately present or adjacent groups thereof are linked to form a substituted or unsubstituted aromatic monocyclic or polycyclic ring,
Ar³ and Ar⁴ are each independently a substituted or unsubstituted C6 to C30 aryl group, a substituted or unsubstituted C2 to C30 heterocyclic group, or a combination thereof,
R^{a}, Ar³, and Ar⁴ are each separately present, or adjacent groups of R^{a}, Ar³, and Ar⁴ are linked to form a substituted or unsubstituted aromatic or heteroaromatic monocyclic or polycyclic ring, and
m5 and m6 are each independently an integer of 1 to 3.

10. The composition for an organic optoelectronic device as claimed in claim 8, wherein:
Chemical Formula 2 is represented by Chemical Formula 2A-XIV or Chemical Formula 2C-I: 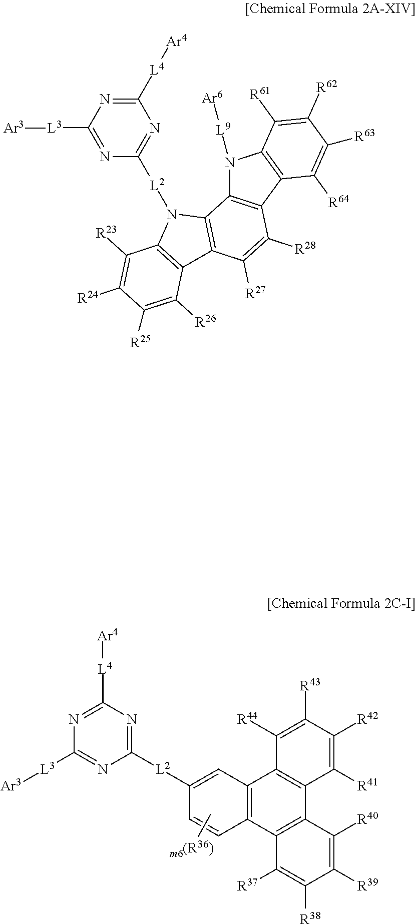
in Chemical Formula 2A-XIV and Chemical Formula 2C-I,
L² to L⁴ and L⁹ are each independently a single bond, a substituted or unsubstituted phenylene group, a substituted or unsubstituted biphenylene group, a substituted or unsubstituted naphthylene group, a substituted or unsubstituted dibenzofuranylene group, or a substituted or unsubstituted dibenzothiopheneylene group,
Ar³, Ar⁴, and Ar⁶ are each independently a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted triphenylene group, a substituted or unsubstituted carbazolyl group, a substituted or unsubstituted dibenzofuranyl group, or a substituted or unsubstituted dibenzothiophenyl group,
R²³ to R²⁸, R³⁶ to R⁴⁴, and R⁶¹ to R⁶⁴ are each independently hydrogen, deuterium cyano group, a substituted or unsubstituted C1 to C10 alkyl group, a substituted or unsubstituted C1 to C10 alkylsilyl group, a substituted or unsubstituted phenyl group, a substituted or unsubstituted dibenzofuranyl group, or a substituted or unsubstituted dibenzothiophenyl group, and
m6 is an integer of 1 to 3.

11. An organic optoelectronic device, comprising:
an anode and a cathode facing each other, and
at least one organic layer between the anode and the cathode,
wherein the at least one organic layer includes the compound for an organic optoelectronic device of claim 1.

12. The organic photoelectronic device as claimed in claim 11, wherein:
the at least one organic layer includes a light emitting layer, and
the light emitting layer includes the compound for an organic optoelectronic device.

13. A display device comprising the organic photoelectronic device as claimed in claim 11.

14. An organic optoelectronic device, comprising:
an anode and a cathode facing each other, and
at least one organic layer between the anode and the cathode,
wherein the at least one organic layer includes the composition for an organic optoelectronic device of claim 8.

15. The organic photoelectronic device as claimed in claim 14, wherein:
the at least one organic layer includes a light emitting layer, and
the light emitting layer includes the composition for an organic optoelectronic device.

16. A display device comprising the organic photoelectronic device as claimed in claim 14.
